SEQUENCE LISTING (5 sequences)

SEQ ID NO: 1 - DNA, 1397 bp, Homo sapiens
  CDS (57)..(1235)
atcccgcgccgacggggctgtgccggagcagctgtgcagagctgcaggcgcgcgtcatggctgctttgagacagccccaggtcgcggagctgctggccgaggcccggcgagccttccgggaggagttcggggccgagcccgagctggccgtgtcagcgccgggccgcgtcaacctcatcggggaacacacggactacaaccagggcctggtgctgcctatggctctggagctcatgacggtgctggtgggcagcccccgcaaggatgggctggtgtctctcctcaccacctctgagggtgccgatgagccccagcggctgcagtttccactgcccacagcccagcgctcgctggagcctgggactcctcggtgggccaactatgtcaagggagtgattcagtactacccagctgcccccctccctggcttcagtgcagtggtggtcagctcagtgcccctggggggtggcctgtccagctcagcatccttggaagtggccacgtacaccttcctccagcagctctgtccagactcgggcacaatagctgcccgcgcccaggtgtgtcagcaggccgagcacagcttcgcagggatgccctgtggcatcatggaccagttcatctcacttatgggacagaaaggccacgcgctgctcattgactgcaggtccttggagaccagcctggtgccactctcggaccccaagctggccgtgctcatcaccaactctaatgtccgccactccctggcctccagcgagtaccctgtgcggcggcgccaatgtgaagaagtggcccgggcgctgggcaaggaaagcctccgggaggtacaactggaagagctagaggctgccagggacctggtgagcaaagagggcttccggcgggcccggcacgtggtgggggagattcggcgcacggcccaggcagcggccgccctgagacgtggcgactacagagcctttggccgcctcatggtggagagccaccgctcactcagagacgactatgaggtgagctgcccagagctggaccagctggtggaggctgcgcttgctgtgcctggggtttatggcagccgcatgacgggcggtggcttcggtggctgcacggtgacactgctggaggcctccgctgctccccacgccatgcggcacatccaggagcactacggcgggactgccaccttctacctctctcaagcagccgatggagccaaggtgctgtgcttgtgaggcacccccaggacagcacacggtgagggtgcggggcctgcaggccagtcccacggctctgtgcccggtgccatcttccatatccgggtgctcaataaacttgtgcctccaatgtggtacctgcctcctctagaggtgggtgtatgcttgggtgtcagagaa

SEQ ID NO: 2 - protein, 392 aa, Homo sapiens
MAALRQPQVAELLAEARRAFREEFGAEPELAVSAPGRVNLIGEHTDYNQGLVLPMALELMTVLVGSPRKDGLVSLLTTSEGADEPQRLQFPLPTAQRSLEPGTPRWANYVKGVIQYYPAAPLPGFSAVVVSSVPLGGGLSSSASLEVATYTFLQQLCPDSGTIAARAQVCQQAEHSFAGMPCGIMDQFISLMGQKGHALLIDCRSLETSLVPLSDPKLAVLITNSNVRHSLASSEYPVRRRQCEEVARALGKESLREVQLEELEAARDLVSKEGFRRARHVVGEIRRTAQAAAALRRGDYRAFGRLMVESHRSLRDDYEVSCPELDQLVEAALAVPGVYGSRMTGGGFGGCTVTLLEASAAPHAMRHIQEHYGGTATFYLSQAADGAKVLCL

SEQ ID NO: 3 - protein, 392 aa, Homo sapiens; identical to SEQ ID NO: 2

SEQ ID NO: 4 - protein, 392 aa, Artificial Sequence
  Synthetic mutant
  MISC_FEATURE (252)..(252): K252A
  MISC_FEATURE (253)..(253): E253A
MAALRQPQVAELLAEARRAFREEFGAEPELAVSAPGRVNLIGEHTDYNQGLVLPMALELMTVLVGSPRKDGLVSLLTTSEGADEPQRLQFPLPTAQRSLEPGTPRWANYVKGVIQYYPAAPLPGFSAVVVSSVPLGGGLSSSASLEVATYTFLQQLCPDSGTIAARAQVCQQAEHSFAGMPCGIMDQFISLMGQKGHALLIDCRSLETSLVPLSDPKLAVLITNSNVRHSLASSEYPVRRRQCEEVARALGAASLREVQLEELEAARDLVSKEGFRRARHVVGEIRRTAQAAAALRRGDYRAFGRLMVESHRSLRDDYEVSCPELDQLVEAALAVPGVYGSRMTGGGFGGCTVTLLEASAAPHAMRHIQEHYGGTATFYLSQAADGAKVLCL

SEQ ID NO: 5 - protein, 399 aa, Artificial Sequence
  Synthetic
  misc_feature (1)..(1): Xaa can be any naturally occurring amino acid
  misc_feature (62)..(62): Xaa can be any naturally occurring amino acid
  misc_feature (67)..(67): Xaa can be any naturally occurring amino acid
  misc_feature (187)..(187): Xaa can be any naturally occurring amino acid
  misc_feature (192)..(192): Xaa can be any naturally occurring amino acid
  misc_feature (199)..(199): Xaa can be any naturally occurring amino acid
  misc_feature (314)..(314): Xaa can be any naturally occurring amino acid
  misc_feature (350)..(350): Xaa can be any naturally occurring amino acid
  misc_feature (372)..(372): Xaa can be any naturally occurring amino acid
XAHHHHHHAALRQPQVAELLAEARRAFREEFGAEPELAVSAPGRVNLIGEHTDYNQGLVLPXALELXTVLVGSPRKDGLVSLLTTSEGADEPQRLQFPLPTAQRSLEPGTPRWANYVKGVIQYYPAAPLPGFSAVVVSSVPLGGGLSSSASLEVATYTFLQQLCPDSGTIAARAQVCQQAEHSFAGXPCGIXDQFISLXGQKGHALLIDCRSLETSLVPLSDPKLAVLITNSNVRHSLASSEYPVRRRQCEEVARALGKESLREVQLEELEAARDLVSKEGFRRARHVVGEIRRTAQAAAALRRGDYRAFGRLXVESHRSLRDDYEVSCPELDQLVEAALAVPGVYGSRXTGGGFGGCTVTLLEASAAPHAXRHIQEHYGGTATFYLSQAADGAKVLCL

(19) United States
(12) Patent Application Publication
(10) Pub. No.: US 2023/0158025 A1
(43) Pub. Date: May 25, 2023

(54) GALACTOKINASE INHIBITORS

(71) Applicants: Li LUI, Rockville, MD (US); Ya-qin ZHANG, Rockville, MD (US); Surendra KARAVADHI, Rockville, MD (US); Juan MARUGAN, Rockville, MD (US); Matthew HALL, Rockville, MD (US); Min SHEN, Rockville, MD (US); Samarjit PATNAIK, Rockville, MD (US); UNIVERSITY OF UTAH RESEARCH FOUNDATION, Salt Lake City, UT (US); NATIONAL INSTITUTES OF HEALTH, Rockville, MD (US)

(72) Inventors: Li Lui, Rockville, MD (US); Ya-qin Zhang, Rockville, MD (US); Surendra Karavadhi, Rockville, MD (US); Juan Marugan, Rockville, MD (US); Matthew Hall, Rockville, MD (US); Min Shen, Rockville, MD (US); Samarjit Patnaik, Rockville, MD (US); Kent Lai, Salt Lake City, UT (US); Manshu Tang, Salt Lake City, UT (US); Francis G. Whitby, Salt Lake City, UT (US); Christopher P. Hill, Salt Lake City, UT (US); Bijina Balakrishna, Salt Lake City, UT (US)

(21) Appl. No.: 17/995,519

(22) PCT Filed: Apr. 22, 2021
(86) PCT No.: PCT/US2021/028551

§ 371 Date: Oct. 5, 2022

Related U.S. Application Data
(60) Provisional application No. 63/014,366, filed on Apr. 23, 2020.

(51) Int. Cl. A61K 31/506; A61K 31/5377; A61K 31/527

(52) U.S. Cl. CPC A61K 31/506; A61K 31/5377; A61K 31/527

(57) ABSTRACT

Described herein are compounds that inhibit galactokinase (GALK) and other kinases and methods for producing the same. Also described are methods for using Structure-Activity Relationships (SAR) to develop compounds with enhanced activity.

### CROSS-REFERENCE TO RELATED APPLICATIONS

This application claims priority to U.S. Provisional Patent Application No. 63/014,366, filed on Apr. 23, 2020, which is incorporated by reference herein in its entirety.

### FEDERALLY SPONSORED RESEARCH

This invention was made with government support under grant numbers HD074844 and HD089933 awarded by the National Institutes of Health. The government has certain rights in the invention.

### REFERENCE TO SEQUENCE LISTING

This application is filed with a Computer Readable Form of a Sequence Listing in accord with 37 C.F.R. § 1.821(c). The text file submitted by EFS-Web, “026389-9280-WO01_sequence_listing_21 Apr. 2021_ST25.txt,” was created on Apr. 21, 2021, contains 5 sequences, has a file size of 21.1 Kbytes, and is hereby incorporated by reference in its entirety.

### TECHNICAL FIELD

Described herein are compounds that inhibit galactokinase (GALK) and other kinases and methods for producing the same. Also described are methods for using Structure-Activity Relationships (SAR) to develop compounds with enhanced activity.

### BACKGROUND

Galactose is an abundant hexose existing as lactose in milk, dairy products, fruits, vegetables, and many other foods. It is metabolized through an evolutionarily conserved pathway referred to as the Leloir pathway (FIG. 1). The first enzyme of the pathway, galactokinase (GALK), converts α-D-galactose to galactose-1-phosphate (gal-1-p). Then, in the presence of the second enzyme, galactose-1-phosphate uridyltransferase (GALT), gal-1-P will react with UDP-glucose to form UDP-galactose and glucose-1-phosphate.

Deficiency of GALT results in a potentially lethal disorder called classic galactosemia (CG). Patients with CG accumulate high level of gal-1-p which can result in severe disease during the newborn period, including liver failure, coagulopathy, coma, and death if not treated. Although removal of galactose from the diet can prevent neonatal death, CG patients still develop chronic complications such as premature ovarian insufficiency (POI), ataxia, speech dyspraxia and mental retardation even in galactose-restricted diet.

The mechanisms for the above chronic complications remain uncertain, but several lines of evidences indicate accumulation of gal-1-p is a major factor that contributes to these complications. Except for cataracts, patients with an inherited deficiency of GALK do not experience the complications observed in GALT-deficient patients. Similarly, while gal7 (i.e., GALT-deficient) mutant yeast stops growing upon galactose challenge, a gal 7 gall double mutant strain (i.e., GALT- and GALK-deficient) is no longer sensitive to galactose. A significant amount of galactose is found in non-dairy foods such as vegetables and fruits, and more importantly, galactose is also produced endogenously from the natural turnover of glycolipids and glycoproteins. Moreover, isotope labeling demonstrated that a 50 kg adult male could produce up to 2 grams of galactose per day.

The PTEN/PI3K/AKT constitutes an important pathway regulating the signaling of multiple biological processes such as apoptosis, metabolism, cell proliferation and cell growth. PTEN is a dual protein/lipid phosphatase which main substrate is the phosphatidyl-inositol,3,4,5 triphosphate (PIP3), the product of PI3K. Increase in PIP3 recruits AKT to the membrane where it is activated by other kinases also dependent on PIP3. It is known that GALK modifies the PTEN/AKT pathway in a number of human tissues and human cell lines. The galactose-1-phosphate produced by GALK feeds into glycolysis. GALK1 is over-expressed in several tumors. Thus, inhibition of GALK may down regulate the PTEN/PI3K/AKT pathway and therefore interfere with tumor growth or development.

Although GALK1 phosphorylates galactose, a six-carbon monosaccharide, it does not belong to the sugar kinase family. It is, in fact, an archetype of the GHMP kinase family (galactokinase, homoserine kinase, mevalonate kinase and phosphomevalonate kinase), which is characterized by a distinct structure compared to other kinase families. All members of the GHMP kinase family have three conserved motifs (I, II and III). Motif II is the most conserved one with a typical sequence of Pro-X-X-X-Gly-Leu-X-Ser-Ser-Ala and is involved in nucleotide binding and catalytic process. The three-dimensional structure of human GALK1 with bound α-D-galactose and Mg-AMPPNP revealed a unique active site geometry associated with the substrate recognition A number of site-directed mutations known to give rise to Type II (GALK1-deficient) galactosemia have been investigated and provided valuable insights in understanding the GALK1 biology at the molecular/structural levels for structure-based drug development.

There is a need for galactokinase enzyme inhibitors useful for the treatment or prophylaxis of diseases associated GALK1 enzyme or the PTEN/PI3K/AKT pathway.

### SUMMARY

One embodiment described herein is a composition for inhibiting a galactokinase activity comprising Formula I or a salt thereof:

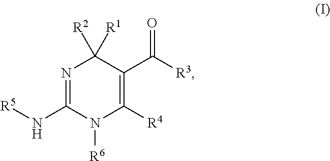

wherein: R¹ and R² are each independently selected from hydrogen, C₆-C₁₂-aryl, C₁-C₆-alkyl, or C₅-C₁₂-heteroaryl, with the proviso that at least one of R¹ or R² is not hydrogen; or where R¹ and R² taken together, including the atoms to which they are attached, form a 5- to 7-membered carbocycle or a 5- to 7-membered heterocycle; R³ is selected from —NH—C₁-C₆-alkyl-C₅-C₁₂-heteroaryl, —NH—C₁-C₆-alkyl-C₆-C₁₂-aryl, —NH—C₁-C₆-alkyl-NH₂, —NH—C₁-C₆-alkyl-NH(C₁₋₄-alkyl), —NH—C₁-C₆-alkyl-N(C₁₋₄-alkyl)₂, or —NR⁷R⁸ wherein R⁷ and R⁸ are each independently selected from hydrogen, C₁-C₆-alkyl, C₃-C₆-cycloalkyl, C₃-C₆-heterocycloalkyl, C₆-C₁₂-aryl, or C₅-C₁₂-heteroaryl; or wherein R⁷ and R⁸ together, including the atoms to which they are attached, form a 5- to 6-membered heteroaryl or a 4- to 6-membered heterocycloalkyl ring; R⁴ is selected from C₁-C₆-alkyl, hydrogen, C₁-C₂-alkoxy-C₁-C₂-alkyl, C₆-C₁₂-aryl, or C₅-C₁₂-heteroaryl; or R⁵ is selected from: substituted benzoxazolyl or substituted benzothioxazolyl, wherein R⁵ is substituted with two, one, three, or four substituents independently selected from —NH₂, halogen, C₁-C₆-alkyl, C₆-C₁₂-aryl, —CF₃, C₁-C₄-alkoxy, C₆-C₁₂-aryloxy, —NH(C₁-C₄-alkyl), —CO₂H, or —N(C₁-C₄-alkyl)₂, with the proviso that R⁵ cannot be substituted with more than one —NH₂ group; or unsubstituted oxazolopyridinyl, unsubstituted tetrahydrobenzo[d]oxazolyl, or unsubstituted 2-phenyloxazolyl; and R⁶ is selected from hydrogen, C₅-C₁₂-heteroaryl, or C₁-C₆-alkyl. In one aspect, R¹ is hydrogen; R² is phenyl or pyridinyl, wherein R² is unsubstituted or substituted with one, two, or three substituents independently selected from halogen, C₁-C₄-alkyl, —CF₃, C₁-C₂-alkoxy, C₆-C₁₂-aryl, C₄-C₆-heterocyclyl, C₅-C₁₂-heteroaryl, —CONH₂, —NH₂, —CN, —CO₂H, or —SO₂NH₂; and R⁵ is selected from: substituted benzoxazolyl or substituted benzothioxazolyl, wherein R⁵ is substituted with one or more substituents independently selected from C₁-C₆-alkyl, halogen, —CF₃, C₁-C₄-alkoxy, —NH₂, or —CO₂H; or unsubstituted oxazolopyridinyl. In another aspect, R⁵ is selected from 4-fluoro-benzoxazol-2-yl, 5-fluoro-benzoxazol-2-yl, 6-fluoro-benzoxazol-2-yl, 7-fluoro-benzoxazol-2-yl, 4-chloro-benzoxazol-2-yl, 5-chloro-benzoxazol-2-yl, 6-chloro-benzoxazol-2-yl, 7-chloro-benzoxazol-2-yl, 5-bromo-benzoxazol-2-yl, 4-methyl-benzoxazol-2-yl, 5-methyl-benzoxazol-2-yl, 6-methyl-benzoxazol-2-yl, 7-methyl-benzoxazol-2-yl, 5-methoxyl-benzoxazol-2-yl, 6-methoxyl-benzoxazol-2-yl, 7-methoxyl-benzoxazol-2-yl, 4-amino-benzoxazol-2-yl, 5-amino-benzoxazol-2-yl, 7-amino-benzoxazol-2-yl, 7-trifluoromethyl-benzoxazol-2-yl, 7-trifluoromethoxyl-benzoxazol-2-yl, 7-nitro-benzoxazol-2-yl, 7-amino-6-fluoro-benzoxazol-2-yl, or 7-carboxylic acid-6-fluoro-benzoxazol-2-yl. In another aspect, R⁵ is selected from 6-fluoro-benzoxazol-2-yl, 5-methyl-benzoxazol-2-yl, 7-methyl benzoxazol-2-yl, or 7-amino-benzoxazol-2-yl, or 7-amino-6-fluoro-benzoxazol-2-yl. In another aspect, R⁵ is 7-amino-6-fluoro-benzoxazol-2-yl.

In another aspect, the compound is selected from: Compounds 1, 2, 3, 4, 5, 6, 9, 10, 11, 12, 13, 14, 15, 16, 17, 18, 22, 23, 285, or 286 as described herein. In another aspect, the compound is selected from: Compounds 1, 45, 46, 47, 48, 49, 50, 51, 52, 61, 62, 63, 64, 66, 67, 75, 76, 77, 78, 287, or 288 as described herein. In another aspect, the compound is selected from: Compounds 57, 58, 60, 65, 69, 70, 71, 72, 73, 73, 74, or 289 as described herein. In another aspect, the compound is selected from: Compounds 102, 103, 104, 105, 106, 107, 108, or 109 as described herein. In another aspect, the compound is selected from: Compounds 79, 80, 83, 89, 90, 91, 92, 110, 111, 112, 113, 114, 124, 290, 291, or 292 as described herein. In another aspect, the compound is selected from: Compounds 82, 118, 120, 125, 126, 127, 129, 130, 131, 132, 133, 136, 293, 294, 295, or 296 as described herein. In another aspect, the compound is selected from: Compounds 93, 94, 95, 97, 100, 101, 297, 298, or 299 as described herein. In another aspect, the compound is selected from: Compounds 26, 27, 28, 29, 30, 33, 34, 35, 36, 37, 38, 39, 40, 41, 42, 43, or 44 as described herein. In another aspect, the compound is Compound 126 as described herein.

Another embodiment described herein is a composition for inhibiting galactokinase activity comprising Formula II or a salt thereof:

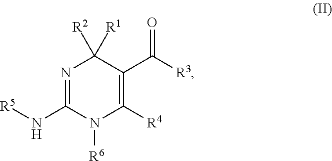

wherein: R¹ is hydrogen; R² is selected from: 2-chlorophenyl optionally substituted with one or more substituents, wherein the optional substituents are independently selected from halogen, —CF₃, C₁-C₆-alkyl, C₁-C₄-alkoxy, C₆-C₁₂-aryloxy, —NH₂, —NH(C₁-C₄-alkyl), —N(C₁-C₄-alkyl)₂, C₃-C₆-cycloalkyl, C₄-C₆-heterocycloalkyl, or C₅-C₁₂-heteroaryl; or phenyl optionally substituted with one or more substituents, wherein the optional substituents are independently selected from —CF₃, —CONH₂, or —SO₂NH₂; R³ is selected from —NH—C₁-C₄-alkyl-C₅-C₁₂-heteroaryl, —NH—C₁-C₄-alkyl-C₆-C₁₂-aryl, morphilino, or —NR⁷R⁸, wherein R⁷ and R⁸ are each independently selected from hydrogen, C₁-C₆-alkyl, C₃-C₆-cycloalkyl, C₆-C₁₂-aryl, C₅-C₁₂-heteroaryl, CO₂Et-C₁-C₆-alkyl, CO₂H—C₁-C₆-alkyl, NH₂—C₁-C₆-alkyl, NH(C₁-C₄-alkyl)-C₁-C₆-alkyl, N(C₁-C₄-alkyl)₂-C₁-C₆-alkyl, C₁-C₂-alkyloxy-C₁-C₆-alkyl, or HO—C₁-C₆-alkyl; or wherein R⁷ and R⁸ together, including the atoms to which they are attached, form a 5- to 6-membered heteroaryl or a 4- to 6-membered heterocycloalkyl ring, with the proviso that R³ is not —NH-p-tolyl; R⁴ is selected from hydrogen, C₁-C₆-alkyl, C₅-C₁₂-heteroaryl, C₁-C₄-alkoxy, or C₁-C₂-alkoxy-C₁-C₂-alkyl; or R³ and R⁴ together form the formula:

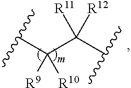

wherein R⁹, R¹⁰, R¹¹, and R¹² are each independently selected from hydrogen, C₁-C₄-alkyl; and m is 0 or 1; R⁵ is selected from unsubstituted C₅-C₁₂-heteroaryl, optionally substituted benzoxazolyl, or optionally substituted benzothioxazolyl, wherein the optional substituents are independently selected from C₁-C₆-alkyl, C₆-C₁₂-aryl, halogen, —CF₃, C₁-C₄-alkoxy, C₆-C₁₂-aryloxy, —NH₂, —NH(C₁-C₄-alkyl), or —N(C₁-C₄-alkyl)₂; and R⁶ is selected from C₁-C₆-alkyl, —CH₂CO₂H, —CO₂Et, or —COPh. In one aspect, R² is unsubstituted 2-chlorophenyl and R³ is selected from —NH—C₆-C₁₂-aryl, —NH—C₁-C₂-alkyl-C₆-C₁₂-aryl, —NH—C₅-C₁₂-heteroaryl, or —NH—C₁-C₂-alkyl-C₅-C₁₂-heteroaryl. In another aspect, R² is 2-chlorophenyl and R³ is selected from —NH-4-benzoic acid, —NH-2-isonicotinic acid, or —NH-((1-methyl-1H-pyrazol-4-yl)methyl).

In another aspect, the compound is selected from: Compounds 137, 138, 139, 140, 141, 142, 143, 144, 146, 147, 150, 152, 156, 159, 160, 163, 169, 170, 171, 172, 176, 177, 178, 179, 181, 182, 183, 184, 189, 190, 191, 201, 210, 300, 301, 302, 303, 304, 305, 306, 307, 308 or 309 as described herein. In another aspect, the compound is selected from: Compounds 145, 164, 165, 166, 188, 194, 195, 196, 202, 203, 204, 211, 212, 213, 310, or 311 as described herein. In another aspect, the compound is selected from: Compounds 174, 215, 216, 217, 219, 220, 221, 222, 223, 224, 225, 226, 227, 228, 229, 230, 231, 232, 233, 234, 237, 238, or 312 as described herein. In another aspect, the compound is selected from: Compounds 167, 168, 173, 174, 180, 185, 186, 187, 193, 198, 199, 200, 205, 261, 264, 266, 269, or 270 as described herein. In another aspect, the compound is selected from: Compounds 246, 247, 248, 249, or 250 as described herein. In another aspect, the compound is selected from: Compounds 273, 274, 275, or 313 as described herein. In another aspect, the compound is selected from: Compounds 145, 151, 245, 314, 315, 316, 317, 321, 322, 323, 324, or 325 as described herein.

Another embodiment described herein is a composition for inhibiting galactokinase activity comprising Formula III or a salt thereof:

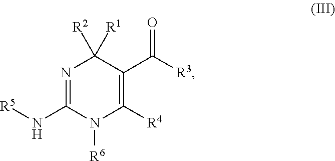

wherein: R¹ and R² are each independently selected from hydrogen, C₁-C₄-alkyl, C₁-C₂-alkoxy, C₁-C₂-hydroxy, C₁-C₂-thioalkyl, C₆-C₁₂-aryl, or C₅-C₁₂-heteroaryl; or R¹ and R² taken together, including the atoms to which they are attached, form a 4- to 8-membered carbocycle or a 4- to 6-membered heterocycle; R³ is selected from —NH—C₅-C₁₂-heteroaryl, —NH—C₁-C₂-alkyl-C₅-C₁₂-heteroaryl, or —NH—C₁-C₂-alkyl-C₃-C₆-heterocycloalkyl; R⁴ is C₁-C₆-alkyl; R⁵ is selected from unsubstituted C₅-C₁₂-heteroaryl, optionally substituted benzoxazolyl, or optionally substituted benzothioxazolyl, wherein the optional substituents are selected from C₁-C₆-alkyl, C₆-C₁₂-aryl, halogen, —CF₃, C₁-C₄-alkoxy, C₆-C₁₂-aryloxy, —NH₂, —NH(C₁-C₄-alkyl), or —N(C₁-C₄-alkyl)₂; and R⁶ is selected from hydrogen or C₁-C₆-alkyl.

In one aspect, the compound is selected from: Compounds 255, 256, 257, 258, 260, 262, 263, 265, 267, 268, 271, 318, 319, or 320 as described herein.

Another embodiment described herein is a compound selected from: Compounds 1-325 as described herein.

Another embodiment described herein is a method for inhibiting a galactokinase in a subject in need thereof, the method comprising administering to the subject an effective amount of a compound described herein, or a pharmaceutically acceptable salt, hydrate, solvate, prodrug, stereoisomer, or tautomer thereof.

Another embodiment described herein is a method for inhibiting a galactokinase in a subject in need thereof, the method comprising administering to the subject a therapeutically effective amount of a pharmaceutical composition comprising a compound described herein, or a pharmaceutically acceptable salt, hydrate, solvate, prodrug, stereoisomer, or tautomer thereof, and optionally, a pharmaceutically acceptable carrier.

Another embodiment described herein is a method for treating or prophylaxis of a disease associated with the galactokinase (GALK1) enzyme or the PTEN/PI3K/AKT pathway in a subject in need thereof, the method comprising administering to the subject a therapeutically effective amount of a compound described herein, or a pharmaceutically acceptable salt, hydrate, solvate, prodrug, stereoisomer, or tautomer thereof, and optionally, a pharmaceutically acceptable carrier.

Another embodiment described herein is a method for treating or prophylaxis of classic galactosemia in a subject in need thereof, the method comprising administering to the subject a therapeutically effective amount of a compound described herein, or a pharmaceutically acceptable salt, hydrate, solvate, prodrug, stereoisomer, or tautomer thereof.

Another embodiment described herein is a method for treating or prophylaxis of liver failure, coagulopathy, coma, or death mediated by a the galactokinase (GALK1) enzyme or the PTEN/PI3K/AKT pathway in a subject in need thereof, the method comprising administering to the subject a therapeutically effective amount of a compound described herein, or a pharmaceutically acceptable salt, hydrate, solvate, prodrug, stereoisomer, or tautomer thereof, and optionally, a pharmaceutically acceptable carrier.

Another embodiment described herein is the use of a compound described herein for treating or prophylaxis of a disease associated with the galactokinase (GALK1) enzyme or the PTEN/PI3K/AKT pathway in a subject in need thereof, the method comprising administering to the subject a therapeutically effective amount of the compound or a pharmaceutically acceptable salt, hydrate, solvate, prodrug, stereoisomer, or tautomer thereof, and optionally, a pharmaceutically acceptable carrier.

Another embodiment described herein is a kit comprising a dosage form of a compound described herein; at least one moisture proof dispensing receptacle comprising blister or strip packs, an aluminum blister, a transparent or opaque polymer blister with pouch, polypropylene tubes, colored blister materials, tubes, bottles, and bottles optionally containing a child-resistant feature, optionally comprising a desiccant, such as a molecular sieve or silica gel; and optionally an insert comprising instructions or prescribing information for the compound or directions for administration or any contraindications.

Another embodiment described herein is a method for manufacturing a compound described herein, the method comprising performing any one of the synthesis reactions described herein.

Another embodiment described herein is a compound produced by any of the methods described herein.

Another embodiment described herein is a method for using Structure-Activity Relationship (SAR) analyses to develop compounds with enhanced activity for inhibiting a galactokinase. In one aspect, the method comprises using any of the compounds described herein.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the Leloir pathway for galactose metabolism.

FIG. 2A-B show the alignment of the co-crystal structure of Compound A (2-(benzo[d]oxazol-2-ylamino)-4-(4-chloro-1H-pyrazol-3-yl)-4,6,7,8-tetrahydroquinazolin-5(1H)-one) and hGALK1 with the structure of hGALK1 in PDB Accession No. 1WUU. All molecules of the PDB Accession No. 1WUU were in magenta and the protein of the co-crystal structure of Compound A and GALK1 was in green. Compound A and the galactose of the co-crystal structure of Compound A and GALK1 were in cyan. FIG. 2A shows an overall view of the alignment of two crystal structures. The yellow circle at the lower left corner indicated the two surface entropy reduction mutants. The chemical structure of Compound A is shown below the structure. FIG. 2B shows a closeup view of Compound A's binding position. The hydrogen bond between Compound A and Arg105 was indicated with red dot line.

FIG. 3A-F show detailed studies of the binding position of Compound A and optimization simulations. Compound A was in cyan and hGALK1 protein was in green. FIG. 3A shows a detailed view of the pocket next to C4 or C7 of the benzoxazole ring. Red dot lines indicated the distance from Ser144, Thr61 and Ser131 to C4 or C7 carbon atom, respectively. FIG. 3B shows a simulation of amine substitution at C4 or C7 of the benzoxazole ring. Red dot lines indicated the distance from Ser144, Thr61 and Ser131 to the amine nitrogen atom, respectively. FIG. 3C shows a detailed view of the pocket next to C5 or C6 of the benzoxazole ring. Red dot lines indicated the distance from Thr61, Ser131, Val130 and Val129 to C5 or C6 carbon atom, respectively. FIG. 3D shows a simulation of fluorine substitution at C5 or C6 of the benzoxazole ring. Red dot lines indicated the distance from Thr61, Ser131, Val130 and Val129 to fluorine atom, respectively. FIG. 3E shows a detailed view of the chiral center. The crystal structure clearly showed only the S-enantiomer of Compound A bound to the protein. Red dot lines indicated the distance from the backbone of Trp106 and Arg105 to chiral carbon atom. FIG. 3F shows a simulation of the R-enantiomer binding to the protein. Only the chiral group was changed to the R-form (part of compound in magenta), which collided with the protein, and all other parts of Compound A were kept unchanged.

FIG. 4A-D show the characterization of Compound 1 and 3 using SPR and the co-crystal structure of Compound 1. FIG. 4A and FIG. 4B show SPR studies for Compounds 1 and 3, respectively. FIG. 4C shows an overview of active site of hGALK1 in the aligned co-crystal structures of Compound 1 and Compound A (from FIG. 3). All molecules for the co-crystal structure of Compound 1 and hGALK1 were colored in blue, and the coloring of the co-crystal structure of Compound A was the same as that of FIG. 3. FIG. 4D shows a close-up view of the alignment at the benzoxazole rings of the two co-crystal structures. The red circle indicated the fluorine atom presented in the co-crystal structure of Compound 1 and hGALK1. FIG. 4E shows hydrogen bonds formed between Compounds A and 1 with the enzyme. Cyan dot lines indicated the hydrogen bonds that Compound 1 formed with Tyr109 and Arg105 respectively, and the red dot line indicated the hydrogen bond that Compound 137 formed with Arg105.

FIG. 5A-B show the effect of Compound 126 on Galactose-1-phosphate (Gal-1P) accumulation. FIG. 5A shows a bar graph illustrating that Compound 126 inhibited Gal-1P accumulation in CG patient fibroblasts. FIG. 5B shows a bar graph illustrating that Compound 126 demonstrated significantly greater inhibition of Gal-1P accumulation when the concentration was increased from 0.5 μM to 1 μM, however when the concentration was increased from 1 μM to 3 μM Gal-1P inhibition did not increase significantly.

FIG. 6A-B show Compound 126 pharmacokinetic (PK) data. FIG. 6A shows a scatterplot graph of the single dose PK data in female CD1 mice (n=3) for different types of administration (IP, IV, and PO). FIG. 6B shows a scatterplot graph of the PK data after 50 mg/kg Compound 126 IP administration in female CD1 mice (n=3).

FIG. 7A-D show the PK blood profile data of Gal-1P and ¹³C₆-Gal-1P formed after administering galactose and ¹³C₆-galactose respectively. FIG. 7A-B show scatterplot graphs of Gal-1P formed after galactose 578 mg/kg IP single dose administration in female CD1 mice (n=5). FIGS. 7A and 7B show identical data with different scales; FIG. 7A has a logarithmic scale and FIG. 7B has a linear scale. FIG. 7C-D show scatterplot graphs of ¹³Cr-Gal-1P formed after ¹³C₆-galactose 572 mg/kg IP single dose administration in female CD1 mice (n=5). FIGS. 7C and 7D show identical data with different scales; FIG. 7C has a logarithmic scale and FIG. 7D has a linear scale.

FIG. 8A-D show the pharmacodynamic (PD) data of ¹³C₆-Gal-1P formed after 50 mg/kg Compound 126, followed (1 hour later) by 400 mg/kg ¹³C₆-galactose IP single dose administration in female CD1 mice. FIG. 8A shows a scatterplot graph of the blood PD data for ¹³C₆-Gal-1P formation in female CD1 mice (n=5). FIG. 8B shows a scatterplot graph of the ovary PD data for ¹³C₆-Gal-1P formation in female CD1 mice (n=5). FIG. 8C shows a scatterplot graph of the liver PD data for ¹³C₆-Gal-1P formation in female CD1 mice (n=5). FIG. 8D shows a scatterplot graph of the brain PD data for ¹³C₆-Gal-1P formation in female CD1 mice (n=5).

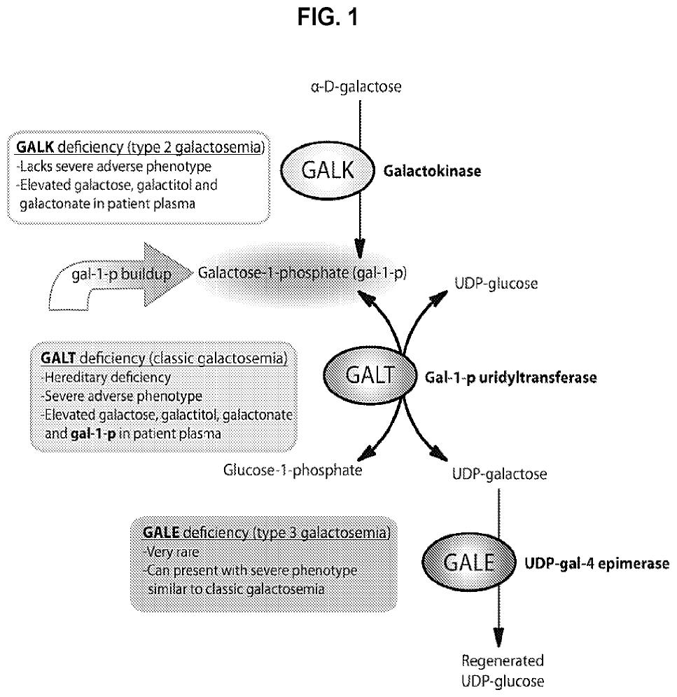

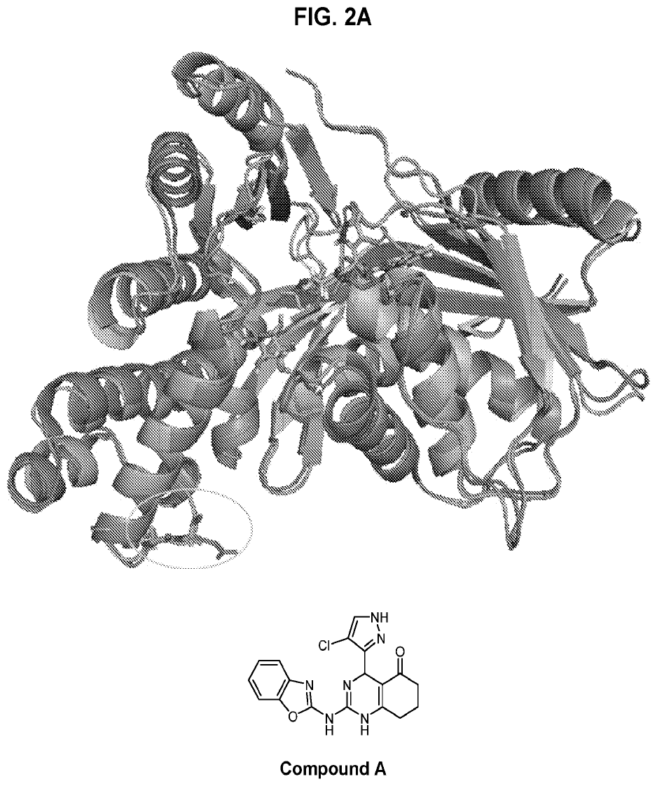

### DETAILED DESCRIPTION

Described herein are inhibitors of galactokinase enzymes.

Using a quantitative high-throughput screening (qHTS) and virtual screening, multiple small molecule inhibitors for human GALK1 have been discovered. Among them, a series of spiro-benzoxazole compounds were identified as selective inhibitors of GALK1 and were shown to lower gal-1P level in primary fibroblast cells derived from patients with classic galactosemia. Further medicinal chemistry optimization of the lead compound resulted in a number of improved inhibitors with low micromolar potency and moderate in vitro ADME and pharmacokinetic properties. Further optimization of this series of compounds was based on the structural information generated by co-crystallizing of GALK1 and the selected compounds. These efforts resulted in low nanomolar enzyme inhibition potency.

As used herein, the term “subject” refers to an animal. Typically, the animal is a mammal. A subject also refers to, for example, primates (e.g., humans, male or female), cows, sheep, goats, horses, dogs, cats, rabbits, rats, mice, fish, birds, and the like. In one embodiment, the subject is a primate. In one embodiment, the subject is a human.

As used herein, the terms “inhibit,” “inhibition,” or “inhibiting” refer to the reduction or suppression of a given condition, symptom, or disorder, or disease, or a significant decrease in the baseline activity of a biological activity or process.

As used herein, the terms “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment “treat,” “treating” or “treatment” refers to alleviating or ameliorating at least one physical parameter including those which may not be discernible by the patient. In yet another embodiment, “treat,” “treating” or “treatment” refers to modulating the disease or disorder, either physically, (e.g., stabilization of a discernible symptom), physiologically, (e.g., stabilization of a physical parameter), or both. In yet another embodiment, “treat,” “treating,” or “treatment” refers to preventing or delaying the onset or development or progression of the disease or disorder.

As used herein, the term “preventing” refers to a reduction in the frequency of, or delay in the onset of, symptoms of the condition or disease.

As used herein, a subject is “in need of” a treatment if such subject would benefit biologically, medically, or in quality of life from such treatment.

The phrase “a therapeutically effective amount” of compounds described herein refers to an amount of the compounds described herein that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of compounds described herein that, when administered to a subject, is effective to (1) at least partially alleviate, inhibit, prevent and/or ameliorate a condition, or a disorder or a disease (i) mediated by galactokinase, or (ii) associated with galactokinase activity, or (iii) characterized by activity (normal or abnormal) of galactokinase; or (2) reduce or inhibit the activity of galactokinase; or (3) reduce or inhibit the expression of galactokinase. In another non-limiting embodiment, the term “a therapeutically effective amount” refers to the amount of compounds described herein that, when administered to a cell, or a tissue, or a non-cellular biological material, or a medium, is effective to at least partially reducing or inhibiting the activity of galactokinase; or at least partially reducing or inhibiting the expression of galactokinase. The meaning of the term “a therapeutically effective amount” as illustrated in the above embodiment for galactokinase also applies by the same means to any other relevant proteins/peptides/enzymes, such as galactose-1-phosphate uridyltransferase, or other enzymes or receptors of the PTEN/PI3K/AKT pathway, and the like.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 6 carbon atoms (“C₁₋₆ alkyl”). In some embodiments, an alkyl group has 1 to 4 carbon atoms (“C₁₋₄ alkyl”).

In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tertiary amyl), and hexyl (C₆) (e.g., n-hexyl).

“Alkylene” refers to a divalent radical of an alkyl group, e.g., —CH₂—, —CH₂CH₂—, and —CH₂CH₂CH₂—.

“Heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain.

In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₃ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC1 alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

“Heteroalkylene” refers to a divalent radical of a heteroalkyl group.

“Alkoxy” or “alkoxyl” refers to an —O-alkyl radical. In some embodiments, the alkoxy groups are methoxy, ethoxy, n-propoxy, isopropoxy, n-butoxy, tert-butoxy, sec-butoxy, n-pentoxy, n-hexoxy, and 1,2-dimethylbutoxy. In some embodiments, alkoxy groups are lower alkoxy, i.e., with between 1 and 6 carbon atoms. In some embodiments, alkoxy groups have between 1 and 4 carbon atoms.

As used herein, the term “aryl” refers to a stable, aromatic, mono- or bicyclic ring radical having the specified number of ring carbon atoms. In one embodiment, the aryl is an aromatic ring having 6 to 16 carbon atoms (“C₆-C₁₆ aryl”). In another embodiment, the aryl is an aromatic ring having 6 to 12 carbon atoms (“C₆-C₁₂ aryl”). In another embodiment, the aryl is an aromatic ring having 6 to 10 carbon atoms (“C₆-C₁₀ aryl”). In another embodiment, the aryl is an aromatic ring having 6 carbon atoms (“C₆ aryl”). Examples of aryl groups include, but are not limited to, phenyl, 1-naphthyl, 2-naphthyl, and the like. The related term “aryl ring” likewise refers to a stable, aromatic, mono- or bicyclic ring having the specified number of ring carbon atoms.

As used herein, the term “heteroaryl” refers to a stable, aromatic, mono- or bicyclic ring radical having the specified number of ring atoms and comprising one or more heteroatoms individually selected from nitrogen, oxygen, and sulfur. The heteroaryl radical may be bonded via a carbon atom or heteroatom. Examples of heteroaryl groups include, but are not limited to, furyl, pyrrolyl, thienyl, pyrazolyl, imidazolyl, thiazolyl, isothiazolyl, oxazolyl, isoxazolyl, triazolyl, tetrazolyl, pyrazinyl, pyridazinyl, pyrimidyl, pyridyl, quinolinyl, isoquinolinyl, indolyl, indazolyl, oxadiazolyl, benzothiazolyl, quinoxalinyl, and the like. The related term “heteroaryl ring” likewise refers to a stable, aromatic, mono- or bicyclic ring having the specified number of ring atoms and comprising one or more heteroatoms individually selected from nitrogen, oxygen, and sulfur.

As used herein, the term “carbocyclyl” refers to a stable, saturated, or unsaturated, non-aromatic, mono- or bicyclic (fused, bridged, or spiro) ring radical having the specified number of ring carbon atoms. Examples of carbocyclyl groups include, but are not limited to, the cycloalkyl groups identified above, cyclobutenyl, cyclopentenyl, cyclohexenyl, and the like. In an embodiment, the specified number is C₃-C₁₂ carbons. The related term “carbocyclic ring” likewise refers to a stable, saturated, or unsaturated, non-aromatic, mono- or bicyclic (fused, bridged, or spiro) ring having the specified number of ring carbon atoms.

As used herein, the term “heterocyclyl” refers to a stable, saturated or unsaturated, non-aromatic, mono- or bicyclic (fused, bridged, or spiro) ring radical having the specified number of ring atoms and comprising one or more heteroatoms individually selected from nitrogen, oxygen and sulfur. The heterocyclyl radical may be bonded via a carbon atom or heteroatom. In an embodiment, the specified number is C₃-C₁₂ carbons. Examples of heterocyclyl groups include, but are not limited to, azetidinyl, oxetanyl, pyrrolinyl, pyrrolidinyl, tetrahydrofuryl, tetrahydrothienyl, piperidyl, piperazinyl, tetrahydropyranyl, morpholinyl, perhydroazepinyl, tetrahydropyridinyl, tetrahydroazepinyl, octahydropyrrolopyrrolyl, and the like. The related term “heterocyclic ring” likewise refers to a stable, saturated or unsaturated, non-aromatic, mono- or bicyclic (fused, bridged, or spiro) ring having the specified number of ring atoms and comprising one or more heteroatoms individually selected from nitrogen, oxygen and sulfur.

As used herein, “spirocycloalkyl” or “spirocyclyl” means carbogenic bicyclic ring systems with both rings connected through a single atom. The rings can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. For example, a (C₃-C₁₂) spirocycloalkyl is a spirocycle containing between 3 and 12 carbon atoms.

As used herein, “spiroheterocycloalkyl” or “spiroheterocyclyl” means a spirocycle wherein at least one of the rings is a heterocycle wherein one or more of the carbon atoms can be substituted with a heteroatom (e.g., one or more of the carbon atoms can be substituted with a heteroatom in at least one of the rings). One or both of the rings in a spiroheterocycle can be fused to another ring carbocyclic, heterocyclic, aromatic, or heteroaromatic ring.

As used herein, “halo” or “halogen” refers to fluorine (fluoro, —F), chlorine (chloro, —Cl), bromine (bromo, —Br), or iodine (iodo, —I).

As used herein, “haloalkyl” means an alkyl group substituted with one or more halogens. Examples of haloalkyl groups include, but are not limited to, trifluoromethyl, difluoromethyl, pentafluoroethyl, and trichloromethyl.

As used herein, “substituted,” whether preceded by the term “optionally” or not, means that one or more hydrogens of the designated moiety are replaced with a suitable substituent.

As used herein, the definition of each expression, e.g., alkyl, m, n, etc., when it occurs more than once in any structure, is intended to be independent of its definition elsewhere in the same structure.

Various embodiments of the disclosure are described herein. It will be recognized that features specified in each embodiment may be combined with other specified features, including as indicated in the embodiments below, to provide further embodiments of the present disclosure.

It is understood that in the following embodiments, combinations of substituents or variables of the depicted formulae are permissible only if such combinations result in stable compounds.

Definitions of specific functional groups and chemical terms are described in more detail below. The chemical elements are identified in accordance with the Periodic Table of the Elements, CAS version, Handbook of Chemistry and Physics, 75^{th} ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} ed, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} ed, Cambridge University Press, Cambridge, 1987.

Certain compounds described herein may exist in particular geometric or stereoisomeric forms. If, for instance, a particular enantiomer of compounds described herein is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Unless otherwise stated, structures depicted herein are also meant to include geometric (or conformational) forms of the structure; for example, the R and S configurations for each asymmetric center, Z and E double bond isomers, and Z and E conformational isomers.

Therefore, single stereochemical isomers as well as enantiomeric, diastereomeric, and geometric (or conformational) mixtures of the disclosed compounds are within the scope of the disclosure.

Unless otherwise stated, all tautomeric forms of the compounds described herein are within the scope of the disclosure. Additionally, unless otherwise stated, structures depicted herein are also meant to include compounds that differ only in the presence of one or more isotopically enriched atoms. For example, compounds having the disclosed structures including the replacement of hydrogen by deuterium or tritium, or the replacement of a carbon by a ¹³C or ¹⁴C enriched carbon are within the scope of this disclosure. Such compounds are useful, for example, as analytical tools, as probes in biological assays, or as therapeutic agents in accordance with the disclosure.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer. ee=(90−10)/100×100=80%.

Thus, a composition containing 90% of one enantiomer and 10% of the other enantiomer is said to have an enantiomeric excess of 80%. The compounds or compositions described herein may contain an enantiomeric excess of at least 50%, 75%, 90%, 95%, or 99% of one form of the compound, e.g., the S-enantiomer. In other words, such compounds or compositions contain an enantiomeric excess of the S enantiomer over the R enantiomer.

Where a particular enantiomer is preferred, it may, in some embodiments be provided substantially free of the corresponding enantiomer and may also be referred to as “optically enriched.” “Optically enriched,” as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high-pressure liquid chromatography (HPLC) and the formation and crystallization of chiral salts or prepared by asymmetric syntheses. See e.g., Jacques et al., Enantiomers, Racemates and Resolutions (Wiley Interscience, New York, 1981); Wilen, et al., Tetrahedron 33:2725 (1977); Eliel, E. L. Stereochemistry of Carbon Compounds (McGraw Hill, N Y, 1962); Wilen, S. H. Tables of Resolving Agents and Optical Resolutions p. 268 (E.L. Eliel, Ed., Univ. of Notre Dame Press, Notre Dame, Ind. 1972).

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound.

In particular, a basic moiety may thus be employed to resolve the compounds described herein into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

As used herein, the term “a,” “an,” “the” and similar terms used in the context of the disclosure (especially in the context of the claims) are to be construed to cover both the singular and plural unless otherwise indicated herein or clearly contradicted by the context.

### Pharmaceutically Acceptable Salts

Pharmaceutically acceptable salts of the compounds described herein are also contemplated for the uses described herein. As used herein, the terms “salt” or “salts” refer to an acid addition or base addition salt of compounds described herein. “Salts” include in particular “pharmaceutical acceptable salts.” The term “pharmaceutically acceptable salts” refers to salts that retain the biological effectiveness and properties of the compounds disclosed herein and, which typically are not biologically or otherwise undesirable. In many cases, the compounds disclosed herein are capable of forming acid and/or base salts by virtue of the presence of amino and/or carboxyl groups or groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium, and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, and tromethamine.

In one embodiment described herein, compounds of Formulae I, II, or III are formulated as acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt forms.

In another embodiment described herein, compounds of Formulae I, II, or III are formulated as sodium, potassium, ammonium, calcium, magnesium, iron, silver, zinc, copper, isopropylamine, benzathine, cholinate, diethanolamine, diethylamine, lysine, meglumine, piperazine or tromethamine salt forms.

In one embodiment described herein, compounds of Formulae I, II, or III can be obtained in the form of their hydrates, or include other solvents used for their crystallization. The compounds described herein may inherently or by design form solvates with pharmaceutically acceptable solvents (including water); therefore, it is intended that the invention embrace both solvated and unsolvated forms. The term “solvate” refers to a molecular complex of compounds described herein (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds described herein, including salts, hydrates, and solvates thereof, may inherently or by design form polymorphs.

### Pharmaceutical Compositions

Another embodiment is a pharmaceutical composition comprising one or more compounds described herein or a pharmaceutically acceptable salt, hydrate, solvate, prodrug, stereoisomer, or tautomer thereof, and one or more pharmaceutically acceptable carrier(s). The term “pharmaceutically acceptable carrier” refers to a pharmaceutically acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions of the disclosure are administered orally, intraperitoneally, or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tween®, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions described herein may be orally administered in any orally acceptable dosage form including, but not limited to, capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring, or coloring agents may also be added.

Alternatively, the pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax, and polyethylene glycols.

The pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be administered in a rectal suppository formulation or in a suitable enema formulation. Topical or transdermal patches may also be used.

For topical applications, the pharmaceutically acceptable compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax, and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

The pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. The amount of the compounds of the present disclosure that may be combined with the carrier materials to produce a composition in a single dosage form will vary depending upon the host treated, the particular mode of administration. Preferably, the compositions should be formulated so that a dosage of between 0.01-100 mg/kg body weight/day of the inhibitor can be administered to a patient receiving these compositions.

### Isotopically Labelled Compounds

Compounds described herein or a pharmaceutically acceptable salt, hydrate, solvate, prodrug, stereoisomer, or tautomer thereof, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds described herein include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F, ³¹P, ³²P ³⁵S, ³⁶Cl, ¹²³I, ¹²⁴I, ¹²⁵I, respectively. The disclosure includes various isotopically labeled compounds as defined herein, for example, those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. An ¹⁸F or labeled compound may be particularly desirable for PET or SPECT studies. Isotopically-labeled compounds described herein or a pharmaceutically acceptable salt, hydrate, solvate, prodrug, stereoisomer, or tautomer thereof, can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of compounds described herein or a pharmaceutically acceptable salt, hydrate, solvate, prodrug, stereoisomer, or tautomer thereof. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in compounds described herein is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Dosages

The pharmaceutical composition or combination described herein can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients.

The therapeutically effective dosage of a compound, the pharmaceutical composition, or the combinations thereof, is dependent on the species of the subject, the body weight, age and individual condition, the disorder or disease or the severity thereof being treated. A physician, clinician, or veterinarian of ordinary skill in the art can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds described herein can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

Toxicity and therapeutic efficacy of compounds described herein, including pharmaceutically acceptable salts and deuterated variants, can be determined by standard pharmaceutical procedures in cell cultures or experimental animals. The LD₅₀ is the dose lethal to 50% of the population. The ED₅₀ is the dose therapeutically effective in 50% of the population. The dose ratio between toxic and therapeutic effects (LD₅₀/ED₅₀) is the therapeutic index. Compounds that exhibit large therapeutic indexes are preferred. While compounds that exhibit toxic side effects may be used, care should be taken to design a delivery system that targets such compounds to the site of affected tissue in order to minimize potential damage to uninfected cells and thereby reduce side effects.

Data obtained from the cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The quantity of compounds described herein in the composition will also depend upon the particular compound in the composition.

### Methods of Use

Another embodiment is a method of inhibiting or modulating a galactokinase in a subject in need thereof, the method comprising administering to the subject a therapeutically effective amount of a compound disclosed herein, or a pharmaceutically acceptable salt, hydrate, solvate, prodrug, stereoisomer, or tautomer thereof.

Another embodiment is a method of inhibiting a galactokinase in a subject in need thereof, the method comprising administering to the subject a therapeutically effective amount of a compound disclosed herein, or a pharmaceutically acceptable salt, hydrate, solvate, prodrug, stereoisomer, or tautomer thereof.

Another embodiment is a method of inhibiting a galactokinase in a subject in need thereof, the method comprising administering to the subject a therapeutically effective amount of a pharmaceutical composition comprising a compound disclosed herein, or a pharmaceutically acceptable salt, hydrate, solvate, prodrug, stereoisomer, or tautomer thereof, and a pharmaceutically acceptable carrier.

Another embodiment is a method of treating or prophylaxis of diseases associated with the galactokinase (GALK1) enzyme or the PTEN/PI3K/AKT pathway in a subject in need thereof, the method comprising administering to the subject a therapeutically effective amount of a compound disclosed herein, or a pharmaceutically acceptable salt, hydrate, solvate, prodrug, stereoisomer, or tautomer thereof.

Another embodiment is a method of treating or prophylaxis of classic galactosemia in a subject in need thereof, the method comprising administering to the subject a therapeutically effective amount of a compound disclosed herein, or a pharmaceutically acceptable salt, hydrate, solvate, prodrug, stereoisomer, or tautomer thereof.

Another embodiment is a method of treating or preventing liver failure, coagulopathy, coma, or death mediated by a the galactokinase (GALK1) enzyme or the PTEN/PI3K/AKT pathway in a subject in need thereof, the method comprising administering to the subject a therapeutically effective amount of a compound disclosed herein, or a pharmaceutically acceptable salt, hydrate, solvate, prodrug, stereoisomer, or tautomer thereof.

### Combination Therapy

Another embodiment is a pharmaceutical combination comprising a compound of Formulae (I), (II), (III) or Compounds 1-325, or a pharmaceutically acceptable salt, hydrate, solvate, prodrug, stereoisomer, or tautomer thereof, and one or more additional therapeutic agent(s) for simultaneous, separate or sequential use in therapy.

In an embodiment, the additional therapeutic agent is selected from one or more of: an antiproliferative agent, anticancer agent, immunomodulatory agent, an anti-inflammatory agent, a neurological treatment agent, an anti-viral agent, an anti-fungal agent, anti-parasitic agent, an antibiotic, or an anti-infective agent.

In another embodiment, the additional therapeutic agent is selected from a second galactokinase inhibitor or other therapeutic agent.

### Methods of Making

The compounds described herein can be prepared in a number of ways well known to those skilled in the art of organic synthesis. By way of example, compounds described herein can be synthesized using the methods described herein, together with synthetic methods known in the art of synthetic organic chemistry, or variations thereon as appreciated by those skilled in the art. Preferred methods include but are not limited to those methods described herein.

### Kits

Another embodiment described herein is a kit comprising one or more pharmaceutical compositions, at least one of which contains a compound of formula a compound of Formulae (I), (II), (III) or Compounds 1-325, or a pharmaceutically acceptable salt, hydrate, solvate, prodrug, stereoisomer, or tautomer thereof. In one embodiment, the kit comprises means for separately retaining said compositions, such as a container, divided bottle, or divided foil packet. An example of such a kit is a blister pack, as typically used for the packaging of tablets, capsules and the like. In one aspect, the kit may be used for administering different dosage forms, for example, oral and parenteral, for administering the separate compositions at different dosage intervals, or for titrating the compositions. To assist compliance, the kit typically comprises a label and instructions for administration.

Another embodiment described herein is a kit for dispensing a pharmaceutical dosage form comprising any of the compounds described herein, the kit comprising: (a) at least one dosage form comprising a compound described herein; (b) at least one moisture proof dispensing receptacle comprising blister or strip packs, an aluminum blister, a transparent or opaque polymer blister with pouch, polypropylene tubes, colored blister materials, tubes, bottles, and bottles optionally containing a child-resistant feature, optionally comprising a desiccant, such as a molecular sieve or silica gel; and optionally (c) an insert comprising instructions or prescribing information for the compound comprised by the oral pharmaceutical composition; or (d) directions for administration or any contraindications.

Another embodiment is a kit comprising one or more pre-filled syringes comprising a solution or suspension of one or more compounds described herein. In one embodiment, such a kit comprises a pre-filled syringe comprising compounds described herein in a blister pack or a sealed sleeve. The blister pack or sleeve may be sterile on the inside. In one aspect, pre-filled syringes as described herein may be placed inside such blister packs or sleeves prior to undergoing sterilization, for example terminal sterilization.

Such a kit may further comprise one or more needles for administration of the compounds described herein. Such kits may further comprise instructions for use, a drug label, contraindications, warnings, or other relevant information. One embodiment described herein is a carton or package comprising one or more pre-filled syringes comprising one or more compounds as described herein contained within a blister pack, a needle, and optionally instructions for administration, a drug label, contraindications, warnings, or other relevant information.

It will be apparent to one of ordinary skill in the relevant art that suitable modifications and adaptations to the compositions, formulations, methods, processes, and applications described herein can be made without departing from the scope of any embodiments or aspects thereof.

The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions, formulations, and methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A composition for inhibiting a galactokinase activity comprising Formula I or a salt thereof:

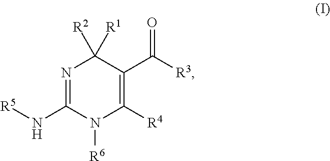

wherein:
R¹ and R² are each independently selected from hydrogen, C₆-C₁₂-aryl, C₁-C₆-alkyl, or C₅-C₁₂-heteroaryl, with the proviso that at least one of R¹ or R² is not hydrogen; or where R¹ and R² taken together, including the atoms to which they are attached, form a 5- to 7-membered carbocycle or a 5- to 7-membered heterocycle;
R³ is selected from —NH—C₁-C₆-alkyl-C₅-C₁₂-heteroaryl, —NH—C₁-C₆-alkyl-C₆-C₁₂-aryl, —NH—C₁-C₆-alkyl-NH₂, —NH—C₁-C₆-alkyl-NH(C₁-C₄-alkyl), —NH—C₁-C₆-alkyl-N(C₁-C₄-alkyl)₂, or —NR⁷R⁸ wherein R⁷ and R⁸ are each independently selected from hydrogen, C₁-C₆-alkyl, C₃-C₆-cycloalkyl, C₃-C₆-heterocycloalkyl, C₆-C₁₂-aryl, or C₅-C₁₂-heteroaryl; or wherein R⁷ and R⁸ together, including the atoms to which they are attached, form a 5- to 6-membered heteroaryl or a 4- to 6-membered heterocycloalkyl ring;
R⁴ is selected from C₁-C₆-alkyl, hydrogen, C₁-C₂-alkoxy-C₁-C₂-alkyl, C₆-C₁₂-aryl, or C₅-C₁₂-heteroaryl; or
R⁵ is selected from:
   substituted benzoxazolyl or substituted benzothioxazolyl, wherein R⁵ is substituted with two, one, three, or four substituents independently selected from —NH₂, halogen, C₁-C₆-alkyl, C₆-C₁₂-aryl, —CF₃, C₁-C₄-alkoxy, C₆-C₁₂-aryloxy, —NH(C₁-C₄-alkyl), —CO₂H, or —N(C₁-C₄-alkyl)₂, with the proviso that R⁵ cannot be substituted with more than one —NH₂ group; or
   unsubstituted oxazolopyridinyl, unsubstituted tetrahydrobenzo[d]oxazolyl, or unsubstituted 2-phenyloxazolyl; and
R⁶ is selected from hydrogen, C₅-C₁₂-heteroaryl, or C₁-C₆-alkyl.
Clause 2. The composition of clause 1, wherein:
   R¹ is hydrogen;
   R² is phenyl or pyridinyl, wherein R² is unsubstituted or substituted with one, two, or three substituents independently selected from halogen, C₁-C₄-alkyl, —CF₃, C₁-C₂-alkoxy, C₆-C₁₂-aryl, C₄-C₆-heterocyclyl, C₅-C₁₂-heteroaryl, —CONH₂, —NH₂, —CN, —CO₂H, or —SO₂NH₂; and
   R⁵ is selected from:
      substituted benzoxazolyl or substituted benzothioxazolyl, wherein R⁵ is substituted with one or more substituents independently selected from C₁-C₅-alkyl, halogen, —CF₃, C₁-C₄-alkoxy, —NH₂, or —CO₂H; or
      unsubstituted oxazolopyridinyl.
Clause 3. The composition of clause 1, wherein R⁵ is selected from 4-fluoro-benzoxazol-2-yl, 5-fluoro-benzoxazol-2-yl, 6-fluoro-benzoxazol-2-yl, 7-fluoro-benzoxazol-2-yl, 4-chloro-benzoxazol-2-yl, 5-chloro-benzoxazol-2-yl, 6-chloro-benzoxazol-2-yl, 7-chloro-benzoxazol-2-yl, 5-bromo-benzoxazol-2-yl, 4-methyl-benzoxazol-2-yl, 5-methyl-benzoxazol-2-yl, 6-methyl-benzoxazol-2-yl, 7-methyl-benzoxazol-2-yl, 5-methoxyl-benzoxazol-2-yl, 6-methoxyl-benzoxazol-2-yl, 7-methoxyl-benzoxazol-2-yl, 4-amino-benzoxazol-2-yl, 5-amino-benzoxazol-2-yl, 7-amino-benzoxazol-2-yl, 7-trifluoromethyl-benzoxazol-2-yl, 7-trifluoromethoxyl-benzoxazol-2-yl, 7-nitro-benzoxazol-2-yl, 7-amino-6-fluoro-benzoxazol-2-yl, or 7-carboxylic acid-6-fluoro-benzoxazol-2-yl.
Clause 4. The composition of clause 1, wherein R⁵ is selected from 6-fluoro-benzoxazol-2-yl, 5-methyl-benzoxazol-2-yl, 7-methyl benzoxazol-2-yl, or 7-amino-benzoxazol-2-yl, or 7-amino-6-fluoro-benzoxazol-2-yl.
Clause 5. The composition of clause 1, wherein R⁵ is 7-amino-6-fluoro-benzoxazol-2-yl.
Clause 6. The composition of clause 1, wherein the compound is selected from: Compounds 1, 2, 3, 4, 5, 6, 9, 10, 11, 12, 13, 14, 15, 16, 17, 18, 22, 23, 285, or 286 as described herein.
Clause 7. The composition of clause 1, wherein the compound is selected from: Compounds 1, 45, 46, 47, 48, 49, 50, 51, 52, 61, 62, 63, 64, 66, 67, 75, 76, 77, 78, 287, or 288 as described herein.
Clause 8. The composition of clause 1, wherein the compound is selected from: Compounds 57, 58, 60, 65, 69, 70, 71, 72, 73, 73, 74, or 289 as described herein.
Clause 9. The composition of clause 1, wherein the compound is selected from: Compounds 102, 103, 104, 105, 106, 107, 108, or 109 as described herein.
Clause 10. The composition of clause 1, wherein the compound is selected from: Compounds 79, 80, 83, 89, 90, 91, 92, 110, 111, 112, 113, 114, 124, 290, 291, or 292 as described herein.
Clause 11. The composition of clause 1, wherein the compound is selected from: Compounds 82, 118, 120, 125, 126, 127, 129, 130, 131, 132, 133, 136, 293, 294, 295, or 296 as described herein.
Clause 12. The composition of clause 1, wherein the compound is selected from: Compounds 93, 94, 95, 97, 100, 101, 297, 298, or 299 as described herein.
Clause 13. The composition of clause 1, wherein the compound is selected from: Compounds 26, 27, 28, 29, 30, 33, 34, 35, 36, 37, 38, 39, 40, 41, 42, 43, or 44 as described herein
Clause 14. The compound of clause 1, wherein the compound is:

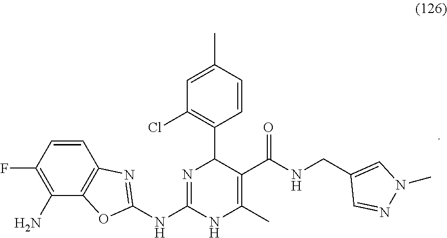 [SMILES: CC1=C(C(=O)NCc2cnn(C)c2)C(c2ccc(C)cc2Cl)N=C(Nc2nc3ccc(F)c(N)c3o2)N1]

Clause 15. A composition for inhibiting galactokinase activity comprising Formula II or a salt thereof:

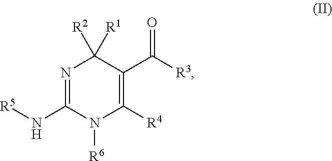

wherein:
R¹ is hydrogen;
R² is selected from:
   2-chlorophenyl optionally substituted with one or more substituents, wherein the optional substituents are independently selected from halogen, —CF₃, C₁-C₆-alkyl, C₁-C₄-alkoxy, C₆-C₁₂-aryloxy, —NH₂, —NH(C₁-C₄-alkyl), —N(C₁-C₄-alkyl)₂, C₃-C₆-cycloalkyl, C₄-C₆-heterocycloalkyl, or C₅-C₁₂-heteroaryl; or
   phenyl optionally substituted with one or more substituents, wherein the optional substituents are independently selected from —CF₃, —CONH₂, or —SO₂NH₂;
R³ is selected from —NH—C₁-C₄-alkyl-C₅-C₁₂-heteroaryl, —NH—C₁-C₄-alkyl-C₆-C₁₂-aryl, morphilino, or —NR⁷R⁸, wherein R⁷ and R⁸ are each independently selected from hydrogen, C₁-C₆-alkyl, C₃-C₆-cycloalkyl, C₆-C₁₂-aryl, C₅-C₁₂-heteroaryl, CO₂Et-C₁-C₆-alkyl, CO₂H—C₁-C₆-alkyl, NH₂—C₁-C₆-alkyl, NH(C₁-C₄-alkyl)-C₁-C₆-alkyl, N(C₁₋₄-alkyl)₂-C₁-C₆-alkyl, C₁-C₂-alkyloxy-C₁-C₆-alkyl, or HO—C₁-C₆-alkyl; or wherein R⁷ and R⁸ together, including the atoms to which they are attached, form a 5- to 6-membered heteroaryl or a 4- to 6-membered heterocycloalkyl ring, with the proviso that R³ is not —NH-p-tolyl;
R⁴ is selected from hydrogen, C₁-C₆-alkyl, C₅-C₁₂-heteroaryl, C₁-C₄-alkoxy, or C₁-C₂-alkoxy-C₁-C₂-alkyl; or
R³ and R⁴ together form the formula:

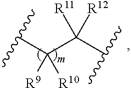

wherein R⁹, R¹⁰, R¹¹, and R¹² are each independently selected from hydrogen, C₁-C₄-alkyl; and m is 0 or 1;
R⁵ is selected from unsubstituted C₅-C₁₂-heteroaryl, optionally substituted benzoxazolyl, or optionally substituted benzothioxazolyl, wherein the optional substituents are independently selected from C₁-C₆-alkyl, C₆-C₁₂-aryl, halogen, —CF₃, C₁-C₄-alkoxy, C₆-C₁₂-aryloxy, —NH₂, —NH(C₁-C₄-alkyl), or —N(C₁-C₄-alkyl)₂; and
R⁶ is selected from C₁-C₆-alkyl, —CH₂CO₂H, —CO₂Et, or —COPh.
Clause 16. The composition of clause 15, wherein R² is unsubstituted 2-chlorophenyl and R³ is selected from —NH—C₆-C₁₂-aryl, —NH—C₁-C₂-alkyl-C₆-C₁₂-aryl, —NH—C₅-C₁₂-heteroaryl, or —NH—C₁-C₂-alkyl-C₅-C₁₂-heteroaryl.
Clause 17. The composition of clause 15, wherein R² is 2-chlorophenyl and R³ is selected from —NH-4-benzoic acid, —NH-2-isonicotinic acid, or —NH-((1-methyl-1H-pyrazol-4-yl)methyl).
Clause 18. The composition of clause 15, wherein the compound is selected from: Compounds 137, 138, 139, 140, 141, 142, 143, 144, 146, 147, 150, 152, 156, 159, 160, 163, 169, 170,171, 172, 176, 177, 178,179, 181, 182, 183, 184, 189, 190, 191, 201, 210, 300, 301, 302, 303, 304, 305, 306, 307, 308 or 309 as described herein.
Clause 19. The composition of clause 15, wherein the compound is selected from: Compounds 145, 164, 165, 166, 188, 194, 195, 196, 202, 203, 204, 211, 212, 213, 310, or 311 as described herein.
Clause 20. The composition of clause 15, wherein the compound is selected from: Compounds 174, 215, 216, 217, 219, 220, 221, 222, 223, 224, 225, 226, 227, 228, 229, 230, 231, 232, 233, 234, 237, 238, or 312 as described herein.
Clause 21. The composition of clause 15, wherein the compound is selected from: Compounds 167, 168, 173, 174, 180, 185, 186, 187, 193, 198, 199, 200, 205, 261, 264, 266, 269, or 270 as described herein.
Clause 22. The composition of clause 15, wherein the compound is selected from: Compounds 246, 247, 248, 249, or 250 as described herein Clause 23. The composition of clause 15, wherein the compound is selected from: Compounds 273, 274, 275, or 313 as described herein.
Clause 24. The composition of clause 15, wherein the compound is selected from: Compounds 145, 151, 245, 314, 315, 316, 317, 321, 322, 323, 324, or 325 as described herein.
Clause 25. A composition for inhibiting galactokinase activity comprising Formula III or a salt thereof:

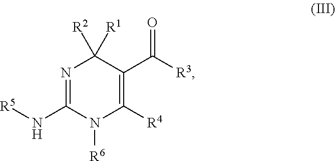

wherein:
R¹ and R² are each independently selected from hydrogen, C₁-C₄-alkyl, C₁-C₂-alkoxy, C₁-C₂-hydroxy, C₁-C₂-thioalkyl, C₆-C₁₂-aryl, or C₅-C₁₂-heteroaryl; or
R¹ and R² taken together, including the atoms to which they are attached, form a 4- to 8-membered carbocycle or a 4- to 6-membered heterocycle;
R³ is selected from —NH—C₅-C₁₂-heteroaryl, —NH—C₁-C₂-alkyl-C₅-C₁₂-heteroaryl, or —NH—C₁-C₂-alkyl-C₃-C₆-heterocycloalkyl;
R⁴ is C₁-C₆-alkyl;
R⁵ is selected from unsubstituted C₅-C₁₂-heteroaryl, optionally substituted benzoxazolyl, or optionally substituted benzothioxazolyl, wherein the optional substituents are selected from C₁-C₆-alkyl, C₆-C₁₂-aryl, halogen, —CF₃, C₁-C₄-alkoxy, C₆-C₁₂-aryloxy, —NH₂, —NH(C₁-C₄-alkyl), or —N(C₁-C₄-alkyl)₂; and
R⁶ is selected from hydrogen or C₁-C₆-alkyl.
Clause 26. The composition of clause 25, wherein the compound is selected from: Compounds 255, 256, 257, 258, 260, 262, 263, 265, 267, 268, 271, 318, 319, or 320 as described herein.
Clause 27. A compound selected from: Compounds 1-325 as described herein.
Clause 28. A method for inhibiting a galactokinase in a subject in need thereof, the method comprising administering to the subject an effective amount of a compound of any one of clauses 1-27, or a pharmaceutically acceptable salt, hydrate, solvate, prodrug, stereoisomer, or tautomer thereof.
Clause 29. A method for inhibiting a galactokinase in a subject in need thereof, the method comprising administering to the subject a therapeutically effective amount of a pharmaceutical composition comprising a compound of any one of clauses 1-27, or a pharmaceutically acceptable salt, hydrate, solvate, prodrug, stereoisomer, or tautomer thereof, and optionally, a pharmaceutically acceptable carrier.
Clause 30. A method for treating or prophylaxis of a disease associated with the galactokinase (GALK1) enzyme or the PTEN/PI3K/AKT pathway in a subject in need thereof, the method comprising administering to the subject a therapeutically effective amount of a compound of any one of clauses 1-27, or a pharmaceutically acceptable salt, hydrate, solvate, prodrug, stereoisomer, or tautomer thereof, and optionally, a pharmaceutically acceptable carrier.
Clause 31. A method for treating or prophylaxis of classic galactosemia in a subject in need thereof, the method comprising administering to the subject a therapeutically effective amount of a compound of any one of clauses 1-27, or a pharmaceutically acceptable salt, hydrate, solvate, prodrug, stereoisomer, or tautomer thereof.
Clause 32. A method for treating or prophylaxis of liver failure, coagulopathy, coma, or death mediated by a the galactokinase (GALK1) enzyme or the PTEN/PI3K/AKT pathway in a subject in need thereof, the method comprising administering to the subject a therapeutically effective amount of a compound of any one of clauses 1-27, or a pharmaceutically acceptable salt, hydrate, solvate, prodrug, stereoisomer, or tautomer thereof, and optionally, a pharmaceutically acceptable carrier.
Clause 33. Use of a compound of any one of clauses 1-27 for treating or prophylaxis of a disease associated with the galactokinase (GALK1) enzyme or the PTEN/PI3K/AKT pathway in a subject in need thereof, the method comprising administering to the subject a therapeutically effective amount of the compound or a pharmaceutically acceptable salt, hydrate, solvate, prodrug, stereoisomer, or tautomer thereof, and optionally, a pharmaceutically acceptable carrier.
Clause 34. A kit comprising a dosage form of a compound of any one of clauses 1-27; at least one moisture proof dispensing receptacle comprising blister or strip packs, an aluminum blister, a transparent or opaque polymer blister with pouch, polypropylene tubes, colored blister materials, tubes, bottles, and bottles optionally containing a child-resistant feature, optionally comprising a desiccant, such as a molecular sieve or silica gel; and optionally an insert comprising instructions or prescribing information for the compound or directions for administration or any contraindications.
Clause 35. A method for manufacturing a compound of any one of clauses 1-27, the method comprising performing any one of the synthesis reactions described herein.
Clause 36. A compound produced by the method of clause 35.
Clause 37. A method for using Structure-Activity Relationship (SAR) analyses to develop compounds with enhanced activity comprising any one of clauses 1-27.

### EXAMPLES

### General Methods

All air- or moisture-sensitive reactions were performed under positive pressure of nitrogen with oven-dried glassware. Anhydrous solvents such as dichloromethane, N,N-dimethylformamide (DMF), acetonitrile, methanol and triethylamine were purchased from Sigma-Aldrich. Preparative purification was performed on a Waters semi-preparative HPLC system. The column used was a Phenomenex Luna C18 (5 μm, 30×75 mm) at a flow rate of 45 mL/min.

Purification was performed using either and Acidic or Basic Standard Gradient method as described:

Acidic Standard Gradient Method:
(10:90 MeCN with 0.1% TFA/deionized water with 0.1% TFA ramped to 100% deionized water with 0.1% TFA).

Basic Standard Gradient Method
(10:90 MeCN with 0.1% NH₄OH/deionized water with 0.1% NH₄OH ramped to 100% deionized water with 0.1% NH₄OH) over 8 minutes unless otherwise noted.

Fraction collection was triggered by UV detection (220 nm). Analytical analysis was performed on an Agilent LC/MS (Agilent Technologies, Santa Clara, Calif.). Purity analysis was determined using a 7-minute gradient of 4% to 100% acetonitrile (containing 0.025% trifluoroacetic acid) in water (containing 0.05% trifluoroacetic acid) with an 8-minute run time at a flow rate of 1 mL/min. A Phenomenex Luna C18 column (3 μm, 3×75 mm) was used at a temperature of 50° C. using an Agilent Diode Array Detector.

Chiral separation was performed on an Agilent 1200 series system. The column used was Chiralpak IA (20 μm, 5×50 cm) at a flow rate of 35 mL/min. The mobile phase 1 consisted of acetonitrile and ethanol (20/80) and the mobile phase 2 consisted of acetonitrile, ethanol and diethylamine (20/80/0.02).

Mass determination was performed using an Agilent 6130 mass spectrometer with electrospray ionization in the positive mode. ¹H NMR spectra were recorded on Varian 400 MHz spectrometers. Chemical shifts are reported in ppm with non-deuterated solvent (DMSO-d5 peak at 2.50 ppm) as internal standard for DMSO-d6 solutions. All the analogs tested in the biological assays have a purity greater than 95% based on LCMS analysis. High resolution mass spectrometry was recorded on Agilent 6210 Time-of-Flight LC/MS system. Confirmation of molecular formulae was accomplished using electrospray ionization in the positive mode with the Agilent Masshunter software (version B.02).

### Example 1

This example demonstrates a synthesis of an exemplary compound in accordance with an embodiment of the invention.

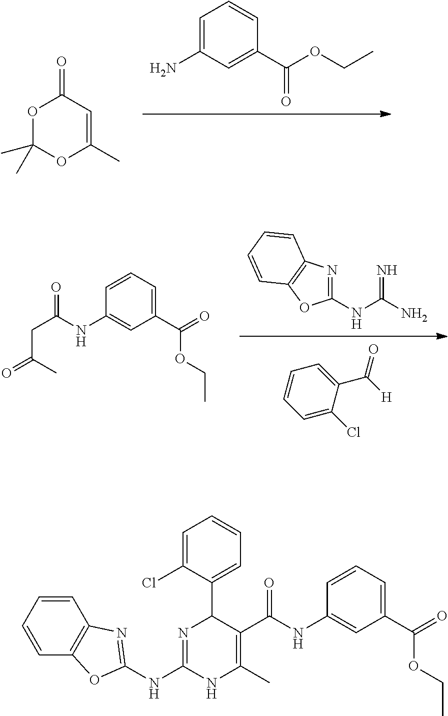

Step 1. The mixture of 2,2,6-trimethyl-4H-1,3-dioxin-4-one (0.3 mL, 2.279 mmol) and ethyl 3-aminobenzoate (0.377 g, 2.279 mmol) in p-xylene (2.3 mL) was sealed and heated 150° C. for 30 min in microwave. The solvent was removed. The crude product was used in the next reaction without further purification. MS m/z (M+H⁺) 250.1.

Step 2. The mixture of ethyl 3-(3-oxobutanamido)benzoate (0.052 g, 0.209 mmol), 2-chlorobenzaldehyde (0.026 mL, 0.229 mmol), and 1-(benzo[d]oxazol-2-yl)guanidine (0.0368 g, 0.209 mmol) was sealed in a microwave tube and heated at 170° C. for 2 hrs. The solvent was removed. MeOH was added to the residue. The crude product was purified. MS m/z (M+H⁺) 530.1. (Compound 139)

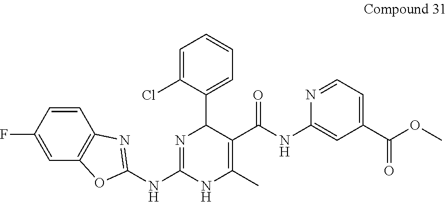 [SMILES: COC(=O)c1ccnc(NC(=O)C2=C(C)NC(Nc3nc4ccc(F)cc4o3)=NC2c2ccccc2Cl)c1]

The title compound (R)-methyl 2-(4-(2-chlorophenyl)-2-((6-fluorobenzo[d]oxazol-2-yl)amino)-6-methyl-1,4-dihydropyrimidine-5-carboxamido)isonicotinate was prepared according to Example 1 using methyl 2-aminoisonicotinate in Step 1 and using 1-(6-fluorobenzo[d]oxazol-2-yl)guanidine in Step 2 as starting material followed by Chiral Separation. MS m/z (M+H⁺) 535.1, ¹H NMR (400 MHz, DMSO-d₆) δ 10.71 (s, 1H), 10.19 (s, 2H), 8.50 (dd, J=5.1, 0.9 Hz, 1H), 8.45 (dd, J=1.5, 0.8 Hz, 1H), 7.58 (dd, J=7.6, 1.9 Hz, 1H), 7.52 (dd, J=5.1, 1.5 Hz, 1H), 7.51-7.48 (m, 1H), 7.43-7.31 (m, 4H), 7.03 (ddd, J=10.2, 8.6, 2.5 Hz, 1H), 6.17 (s, 1H), 3.87 (s, 3H), 2.27 (d, J=0.9 Hz, 3H).

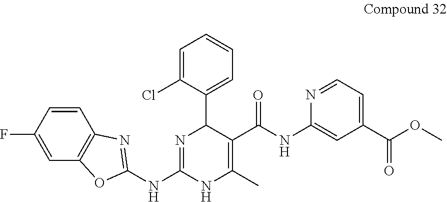 [SMILES: COC(=O)c1ccnc(NC(=O)C2=C(C)NC(Nc3nc4ccc(F)cc4o3)=NC2c2ccccc2Cl)c1]

The title compound (S)-methyl 2-(4-(2-chlorophenyl)-2-((6-fluorobenzo[d]oxazol-2-yl)amino)-6-methyl-1,4-dihydropyrimidine-5-carboxamido)isonicotinate was prepared according to Example 1 using methyl 2-aminoisonicotinate in Step 1 and using 1-(6-fluorobenzo[d]oxazol-2-yl)guanidine in Step 2 as starting material followed by Chiral Separation. MS m/z (M+H⁺) 535.1, ¹H NMR (400 MHz, DMSO-d₆) δ 10.71 (s, 1H), 10.19 (s, 2H), 8.50 (dd, J=5.1, 0.8 Hz, 1H), 8.45 (dd, J=1.5, 0.9 Hz, 1H), 7.58 (dd, J=7.6, 1.9 Hz, 1H), 7.52 (dd, J=5.1, 1.5 Hz, 1H), 7.51-7.48 (m, 1H), 7.42-7.31 (m, 4H), 7.03 (ddd, J=10.2, 8.6, 2.5 Hz, 1H), 6.17 (s, 1H), 3.87 (s, 3H), 2.27 (d, J=0.9 Hz, 3H).

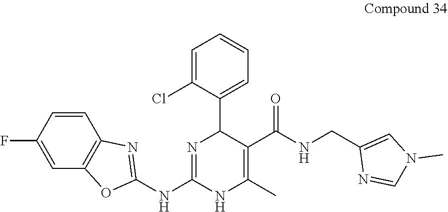 [SMILES: CC1=C(C(=O)NCc2cn(C)cn2)C(c2ccccc2Cl)N=C(Nc2nc3ccc(F)cc3o2)N1]

The title compound 4-(2-chlorophenyl)-2-((6-fluorobenzo[d]oxazol-2-yl)amino)-6-methyl-N-((1-methyl-1H-imidazol-4-yl)methyl)-1,4-dihydropyrimidine-5-carboxamide was prepared according to Example 1 using (1-methyl-1H-imidazol-4-yl)methanamine in Step 1 and using 1-(6-fluorobenzo[d]oxazol-2-yl)guanidine in Step 2 as starting material. MS m/z (M+H⁺) 494.1

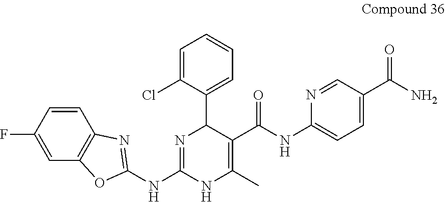 [SMILES: CC1=C(C(=O)Nc2ccc(C(N)=O)cn2)C(c2ccccc2Cl)N=C(Nc2nc3ccc(F)cc3o2)N1]

The title compound N-(5-carbamoylpyridin-2-yl)-4-(2-chlorophenyl)-2-((6-fluorobenzo[d]oxazol-2-yl)amino)-6-methyl-1,4-dihydropyrimidine-5-carboxamide was prepared according to Example 1 using 6-aminonicotinamide in Step 1 and using 1-(6-fluorobenzo[d]oxazol-2-yl)guanidine in Step 2 as starting material. MS m/z (M+H⁺) 520.1

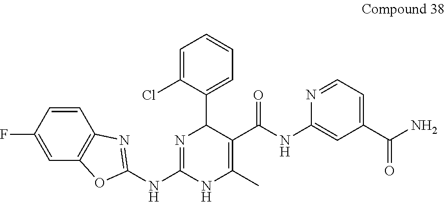 [SMILES: CC1=C(C(=O)Nc2cc(C(N)=O)ccn2)C(c2ccccc2Cl)N=C(Nc2nc3ccc(F)cc3o2)N1]

The title compound N-(4-carbamoylpyridin-2-yl)-4-(2-chlorophenyl)-2-((6-fluorobenzo[d]oxazol-2-yl)amino)-6-methyl-1,4-dihydropyrimidine-5-carboxamide was prepared according to Example 1 using 2-aminonicotinamide in Step 1 and using 1-(6-fluorobenzo[d]oxazol-2-yl)guanidine in Step 2 as starting material. MS m/z (M+H⁺) 520.1

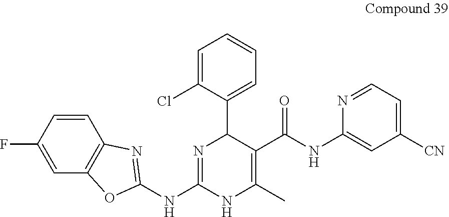 [SMILES: CC1=C(C(=O)Nc2cc(C#N)ccn2)C(c2ccccc2Cl)N=C(Nc2nc3ccc(F)cc3o2)N1]

The title compound 4-(2-chlorophenyl)-N-(4-cyanopyridin-2-yl)-2-((6-fluorobenzo[d]oxazol-2-yl)amino)-6-methyl-1,4-dihydropyrimidine-5-carboxamide was prepared according to Example 1 using 2-aminoisonicotinonitrile in Step 1 and using 1-(6-fluorobenzo[d]oxazol-2-yl)guanidine in Step 2 as starting material. MS m/z (M+H⁺) 502.1, ¹H NMR (400 MHz, DMSO-d₆) δ 10.88 (s, 1H), 10.22 (d, J=1.8 Hz, 1H), 10.19 (t, J=2.5 Hz, 1H), 8.54 (dd, J=5.1, 0.9 Hz, 1H), 8.22 (dd, J=1.4, 1.0 Hz, 1H), 7.54 (dd, J=7.6, 1.9 Hz, 1H), 7.51 (dd, J=5.1, 1.4 Hz, 1H), 7.49-7.46 (m, 1H), 7.39 (dd, J=8.6, 2.4 Hz, 1H), 7.37-7.30 (m, 3H), 7.06-6.97 (m, 1H), 6.13 (d, J=2.8 Hz, 1H), 2.24 (s, 3H).

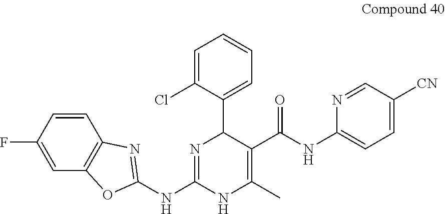 [SMILES: CC1=C(C(=O)Nc2ccc(C#N)cn2)C(c2ccccc2Cl)N=C(Nc2nc3ccc(F)cc3o2)N1]

The title compound 4-(2-chlorophenyl)-N-(5-cyanopyridin-2-yl)-2-((6-fluorobenzo[d]oxazol-2-yl)amino)-6-methyl-1,4-dihydropyrimidine-5-carboxamide was prepared according to Example 1 using 6-aminonicotinonitrile in Step 1 and using 1-(6-fluorobenzo[d]oxazol-2-yl)guanidine in Step 2 as starting material. MS m/z (M+H⁺) 502.1, ¹H NMR (400 MHz, DMSO-d₆) δ 10.90 (s, 1H), 10.22 (s, 1H), 10.17 (d, J=3.0 Hz, 1H), 8.75 (dd, J=2.3, 0.9 Hz, 1H), 8.16 (dd, J=8.8, 2.3 Hz, 1H), 8.03 (dd, J=8.8, 0.8 Hz, 1H), 7.53 (dd, J=7.5, 1.9 Hz, 1H), 7.47 (dd, J=7.6, 1.6 Hz, 1H), 7.40-7.28 (m, 4H), 7.00 (ddd, J=10.2, 8.6, 2.5 Hz, 1H), 6.14 (d, J=3.1 Hz, 1H), 2.24 (s, 3H).

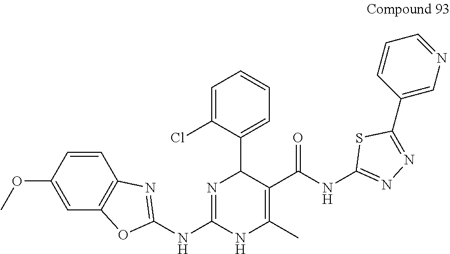 [SMILES: COc1ccc2nc(NC3=NC(c4ccccc4Cl)C(C(=O)Nc4nnc(-c5cccnc5)s4)=C(C)N3)oc2c1]

The title compound 4-(2-chlorophenyl)-2-((6-methoxybenzo[d]oxazol-2-yl)amino)-6-methyl-N-(5-(pyridin-3-yl)-1,3,4-thiadiazol-2-yl)-1,4-dihydropyrimidine-5-carboxamide was prepared according to Example 1 using 3-oxo-N-(5-(pyridin-3-yl)-1,3,4-thiadiazol-2-yl)butanamide in Step 1 and using 1-(6-methoxybenzo[d]oxazol-2-yl)guanidine in Step 2 as starting material. MS m/z (M+H⁺) 573.0

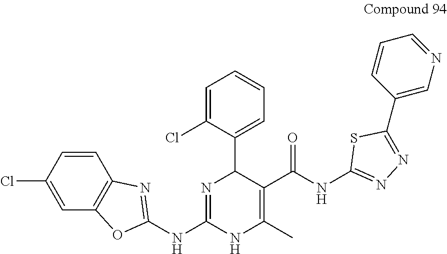 [SMILES: CC1=C(C(=O)Nc2nnc(-c3cccnc3)s2)C(c2ccccc2Cl)N=C(Nc2nc3ccc(Cl)cc3o2)N1]

The title compound 2-((6-chlorobenzo[d]oxazol-2-yl)amino)-4-(2-chlorophenyl)-6-methyl-N-(5-(pyridin-3-yl)-1,3,4-thiadiazol-2-yl)-1,4-dihydropyrimidine-5-carboxamide was prepared according to Example 1 using 3-oxo-N-(5-(pyridin-3-yl)-1,3,4-thiadiazol-2-yl)butanamide in Step 1 and using 1-(6-chlorobenzo[d]oxazol-2-yl)guanidine in Step 2 as starting material. MS m/z (M+H⁺) 577.0, ¹H NMR (400 MHz, DMSO-d₆) δ 12.71 (s, 1H), 10.45 (s, 1H), 10.30 (d, J=2.8 Hz, 1H), 9.16-9.03 (m, 1H), 8.69 (dd, J=4.8, 1.6 Hz, 1H), 8.29 (d, J=8.0 Hz, 1H), 7.66-7.49 (m, 3H), 7.49-7.29 (m, 4H), 7.23 (dd, J=8.4, 2.0 Hz, 1H), 6.26 (d, J=3.1 Hz, 1H), 2.34 (d, J=9.8 Hz, 3H).

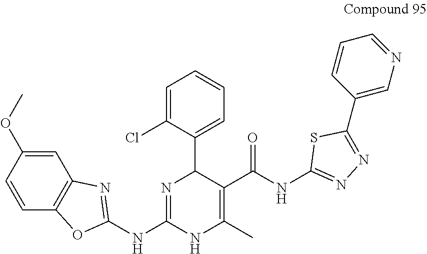 [SMILES: COc1ccc2oc(NC3=NC(c4ccccc4Cl)C(C(=O)Nc4nnc(-c5cccnc5)s4)=C(C)N3)nc2c1]

The title compound 4-(2-chlorophenyl)-2-((5-methoxybenzo[d]oxazol-2-yl)amino)-6-methyl-N-(5-(pyridin-3-yl)-1,3,4-thiadiazol-2-yl)-1,4-dihydropyrimidine-5-carboxamide was prepared according to Example 1 using 3-oxo-N-(5-(pyridin-3-yl)-1,3,4-thiadiazol-2-yl)butanamide in Step 1 and using 1-(5-methoxybenzo[d]oxazol-2-yl)guanidine in Step 2 as starting material. MS m/z (M+H⁺) 573.0

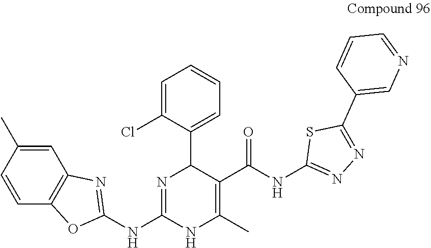 [SMILES: CC1=C(C(=O)Nc2nnc(-c3cccnc3)s2)C(c2ccccc2Cl)N=C(Nc2nc3cc(C)ccc3o2)N1]

The title compound 4-(2-chlorophenyl)-2-((5-methylbenzo[d]oxazol-2-yl)amino)-6-methyl-N-(5-(pyridin-3-yl)-1,3,4-thiadiazol-2-yl)-1,4-dihydropyrimidine-5-carboxamide was prepared according to Example 1 using 3-oxo-N-(5-(pyridin-3-yl)-1,3,4-thiadiazol-2-yl)butanamide in Step 1 and using 1-(5-methylbenzo[d]oxazol-2-yl)guanidine in Step 2 as starting material. MS m/z (M+H⁺) 557.1, ¹H NMR (400 MHz, DMSO-d₆) δ 12.69 (s, 1H), 10.61-10.20 (m, 2H), 9.09 (d, J=2.3 Hz, 1H), 8.70 (dd, J=4.8, 1.6 Hz, 1H), 8.32 (dt, J=8.1, 1.9 Hz, 1H), 7.61-7.49 (m, 2H), 7.44 (s, 1H), 7.36 (td, J=7.0, 1.9 Hz, 2H), 7.28 (d, J=8.1 Hz, 1H), 7.21 (dt, J=1.6, 0.7 Hz, 1H), 7.05-6.81 (m, 1H), 6.26 (d, J=3.0 Hz, 1H), 2.36 (s, 6H).

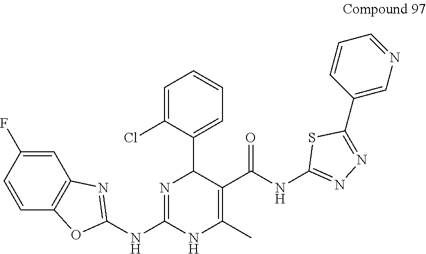 [SMILES: CC1=C(C(=O)Nc2nnc(-c3cccnc3)s2)C(c2ccccc2Cl)N=C(Nc2nc3cc(F)ccc3o2)N1]

The title compound 4-(2-chlorophenyl)-2-((5-fluorobenzo[d]oxazol-2-yl)amino)-6-methyl-N-(5-(pyridin-3-yl)-1,3,4-thiadiazol-2-yl)-1,4-dihydropyrimidine-5-carboxamide was prepared according to Example 1 using 3-oxo-N-(5-(pyridin-3-yl)-1,3,4-thiadiazol-2-yl)butanamide in Step 1 and using 1-(5-fluorobenzo[d]oxazol-2-yl)guanidine in Step 2 as starting material. MS m/z (M+H⁺) 561.0, ¹H NMR (400 MHz, DMSO-d₆) δ 12.70 (s, 1H), 10.46 (s, 1H), 10.32 (d, J=2.7 Hz, 1H), 9.09 (d, J=2.3 Hz, 1H), 8.69 (dd, J=4.8, 1.6 Hz, 1H), 8.30 (dt, J=8.0, 2.0 Hz, 1H), 7.63-7.48 (m, 2H), 7.48-7.29 (m, 4H), 7.24 (dd, J=9.0, 2.6 Hz, 1H), 6.94 (ddd, J=9.9, 8.7, 2.7 Hz, 1H), 6.26 (d, J=3.1 Hz, 1H), 2.35 (q, J=5.3, 4.5 Hz, 3H).

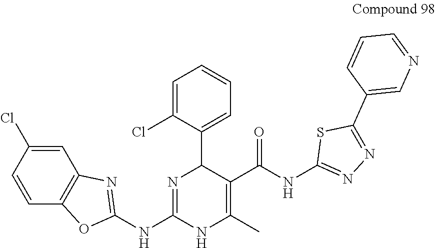 [SMILES: CC1=C(C(=O)Nc2nnc(-c3cccnc3)s2)C(c2ccccc2Cl)N=C(Nc2nc3cc(Cl)ccc3o2)N1]

The title compound 4-(2-chlorophenyl)-2-((5-chlorobenzo[d]oxazol-2-yl)amino)-6-methyl-N-(5-(pyridin-3-yl)-1,3,4-thiadiazol-2-yl)-1,4-dihydropyrimidine-5-carboxamide was prepared according to Example 1 using 3-oxo-N-(5-(pyridin-3-yl)-1,3,4-thiadiazol-2-yl)butanamide in Step 1 and using 1-(5-chlorobenzo[d]oxazol-2-yl)guanidine in Step 2 as starting material. MS m/z (M+H⁺) 577.0, ¹H NMR (400 MHz, DMSO-d₆) δ 12.71 (s, 1H), 10.48 (s, 1H), 10.33 (d, J=2.8 Hz, 1H), 9.24-9.00 (m, 1H), 8.69 (dd, J=4.8, 1.6 Hz, 1H), 8.30 (d, J=8.3 Hz, 1H), 7.65-7.24 (m, 7H), 7.14 (dd, J=8.5, 2.2 Hz, 1H), 6.27 (d, J=3.1 Hz, 1H), 2.41-2.22 (m, 3H).

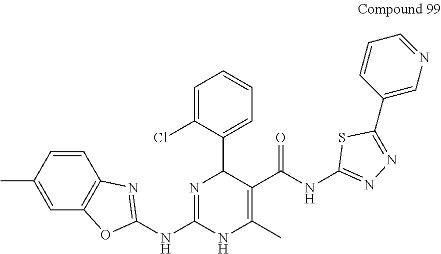 [SMILES: CC1=C(C(=O)Nc2nnc(-c3cccnc3)s2)C(c2ccccc2Cl)N=C(Nc2nc3ccc(C)cc3o2)N1]

The title compound 4-(2-chlorophenyl)-2-((6-methylbenzo[d]oxazol-2-yl)amino)-6-methyl-N-(5-(pyridin-3-yl)-1,3,4-thiadiazol-2-yl)-1,4-dihydropyrimidine-5-carboxamide was prepared according to Example 1 using 3-oxo-N-(5-(pyridin-3-yl)-1,3,4-thiadiazol-2-yl)butanamide in Step 1 and using 1-(6-methylbenzo[d]oxazol-2-yl)guanidine in Step 2 as starting material. MS m/z (M+H⁺) 557.1, ¹H NMR (400 MHz, DMSO-d₆) δ 12.67 (s, 1H), 10.38 (s, 2H), 9.09 (d, J=2.2 Hz, 1H), 8.69 (dd, J=4.8, 1.6 Hz, 1H), 8.30 (d, J=8.0 Hz, 1H), 7.59-7.48 (m, 2H), 7.46 (d, J=7.3 Hz, 1H), 7.36 (qd, J=7.6, 5.9 Hz, 2H), 7.30-7.23 (m, 2H), 7.04-6.98 (m, 1H), 6.25 (d, J=2.8 Hz, 1H), 2.36 (d, J=5.8 Hz, 6H).

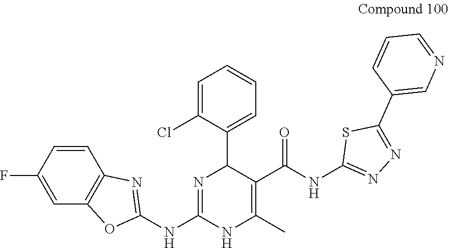 [SMILES: CC1=C(C(=O)Nc2nnc(-c3cccnc3)s2)C(c2ccccc2Cl)N=C(Nc2nc3ccc(F)cc3o2)N1]

The title compound 4-(2-chlorophenyl)-2-((6-fluorobenzo[d]oxazol-2-yl)amino)-6-methyl-N-(5-(pyridin-3-yl)-1,3,4-thiadiazol-2-yl)-1,4-dihydropyrimidine-5-carboxamide was prepared according to Example 1 using 3-oxo-N-(5-(pyridin-3-yl)-1,3,4-thiadiazol-2-yl)butanamide in Step 1 and using 1-(6-fluorobenzo[d]oxazol-2-yl)guanidine in Step 2 as starting material. MS m/z (M+H⁺) 561.1

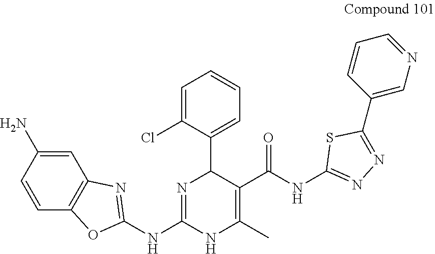 [SMILES: CC1=C(C(=O)Nc2nnc(-c3cccnc3)s2)C(c2ccccc2Cl)N=C(Nc2nc3cc(N)ccc3o2)N1]

The title compound 2-((5-aminobenzo[d]oxazol-2-yl)amino)-4-(2-chlorophenyl)-6-methyl-N-(5-(pyridin-3-yl)-1,3,4-thiadiazol-2-yl)-1,4-dihydropyrimidine-5-carboxamide was prepared according to Example 1 using 3-oxo-N-(5-(pyridin-3-yl)-1,3,4-thiadiazol-2-yl)butanamide in Step 1 and using 1-(5-aminobenzo[d]oxazol-2-yl)guanidine in Step 2 as starting material. MS m/z (M+H⁺) 558.1

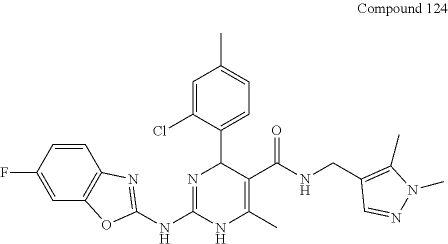 [SMILES: CC1=C(C(=O)NCc2cnn(C)c2C)C(c2ccc(C)cc2Cl)N=C(Nc2nc3ccc(F)cc3o2)N1]

The title compound 4-(2-chloro-4-methylphenyl)-N-((1,5-dimethyl-1H-pyrazol-4-yl)methyl)-2-((6-fluorobenzo[d]oxazol-2-yl)amino)-6-methyl-1,4-dihydropyrimidine-5-carboxamide was prepared according to Example 1 using (1,5-dimethyl-1H-pyrazol-4-yl)methanamine in Step 1 and using 1-(6-fluorobenzo[d]oxazol-2-yl)guanidine and 2-chloro-4-methylbenzaldehyde in Step 2 as starting material. MS m/z (M+H⁺) 522.2, ¹H NMR (400 MHz, DMSO-d₆) δ 10.01 (s, 1H), 9.85 (s, 1H), 8.05 (t, J=5.5 Hz, 1H), 7.33 (dd, J=8.5, 2.4 Hz, 1H), 7.30-7.22 (m, 3H), 7.09 (d, J=7.9 Hz, 1H), 7.03 (s, 1H), 7.01-6.92 (m, 1H), 5.90-5.85 (m, 1H), 3.97 (t, J=4.6 Hz, 2H), 3.61 (s, 3H), 2.24 (s, 3H), 2.08 (s, 3H), 2.02 (s, 3H).

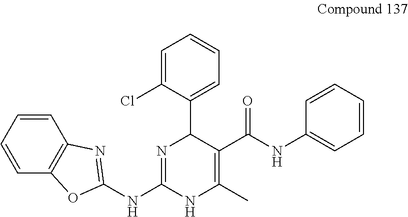 [SMILES: CC1=C(C(=O)Nc2ccccc2)C(c2ccccc2Cl)N=C(Nc2nc3ccccc3o2)N1]

The title compound 2-(benzo[d]oxazol-2-ylamino)-4-(2-chlorophenyl)-6-methyl-N-phenyl-1,4-dihydropyrimidine-5-carboxamide was prepared according to Example 1 using aniline in Step 1 as starting material. MS m/z (M+H⁺) 458.1

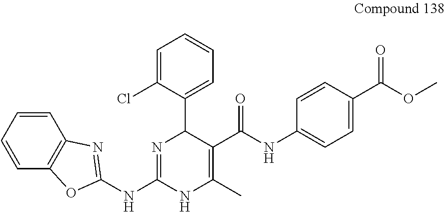 [SMILES: COC(=O)c1ccc(NC(=O)C2=C(C)NC(Nc3nc4ccccc4o3)=NC2c2ccccc2Cl)cc1]

The title compound methyl 4-(2-(benzo[d]oxazol-2-ylamino)-4-(2-chlorophenyl)-6-methyl-1,4-dihydropyrimidine-5-carboxamido)benzoate was prepared according to Example 1 using methyl 4-aminobenzoate in Step 1 as starting material. MS m/z (M+H⁺) 516.1, ¹H NMR (400 MHz, DMSO-d₆) δ 10.32 (d, J=2.9 Hz, 1H), 10.22 (s, 1H), 10.19 (d, J=1.8 Hz, 1H), 7.91-7.85 (m, 2H), 7.73-7.67 (m, 2H), 7.50 (ddd, J=9.5, 7.8, 1.6 Hz, 2H), 7.44-7.30 (m, 4H), 7.17 (td, J=7.6, 1.3 Hz, 1H), 7.13-7.07 (m, 1H), 6.10 (dd, J=3.1, 1.1 Hz, 1H), 3.81 (s, 3H), 2.24 (d, J=0.9 Hz, 3H).

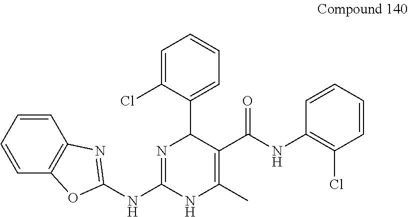 [SMILES: CC1=C(C(=O)Nc2ccccc2Cl)C(c2ccccc2Cl)N=C(Nc2nc3ccccc3o2)N1]

The title compound 2-(benzo[d]oxazol-2-ylamino)-N,4-bis(2-chlorophenyl)-6-methyl-1,4-dihydropyrimidine-5-carboxamide was prepared according to Example 1 using 2-chloroaniline in Step 1 as starting material. MS m/z (M+H⁺) 492.1, ¹H NMR (400 MHz, DMSO-d₆) δ 10.32 (d, J=3.1 Hz, 1H), 10.17 (s, 1H), 9.41 (s, 1H), 7.57-7.50 (m, 2H), 7.49 (dd, J=8.0, 1.6 Hz, 1H), 7.45 (dd, J=8.0, 1.5 Hz, 1H), 7.42-7.33 (m, 4H), 7.31-7.25 (m, 1H), 7.18 (tt, J=7.7, 1.6 Hz, 2H), 7.10 (td, J=7.7, 1.3 Hz, 1H), 6.11 (dd, J=3.0, 1.2 Hz, 1H), 2.35 (d, J=0.9 Hz, 3H).

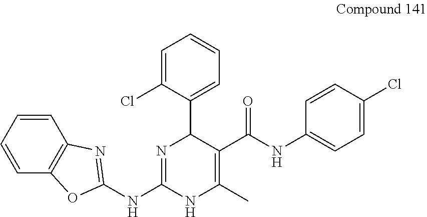 [SMILES: CC1=C(C(=O)Nc2ccc(Cl)cc2)C(c2ccccc2Cl)N=C(Nc2nc3ccccc3o2)N1]

The title compound 2-(benzo[d]oxazol-2-ylamino)-4-(2-chlorophenyl)-N-(4-chlorophenyl)-6-methyl-1,4-dihydropyrimidine-5-carboxamide was prepared according to Example 1 using 4-chloroaniline in Step 1 as starting material. MS m/z (M+H⁺) 492.1, ¹H NMR (400 MHz, DMSO-d₆) δ 10.29 (s, 1H), 10.15 (d, J=1.8 Hz, 1H), 10.02 (s, 1H), 7.60-7.55 (m, 2H), 7.53-7.47 (m, 2H), 7.42-7.30 (m, 6H), 7.17 (td, J=7.6, 1.2 Hz, 1H), 7.14-7.07 (m, 1H), 6.15-6.01 (m, 1H), 2.22 (d, J=0.8 Hz, 3H).

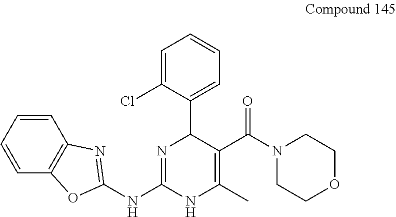 [SMILES: CC1=C(C(=O)N2CCOCC2)C(c2ccccc2Cl)N=C(Nc2nc3ccccc3o2)N1]

The title compound (2-(benzo[d]oxazol-2-ylamino)-4-(2-chlorophenyl)-6-methyl-1,4-dihydropyrimidin-5-yl)(morpholino)methanone was prepared according to Example 1 using 1-morpholinobutane-1,3-dione in Step 2 as starting material. MS m/z (M+H⁺) 452.1, ¹H NMR (400 MHz, DMSO-d₆) δ 10.03 (s, 1H), 9.79 (s, 1H), 7.48 (dd, J=7.0, 2.2 Hz, 1H), 7.46-7.42 (m, 1H), 7.41-7.35 (m, 3H), 7.32 (ddd, J=7.8, 1.4, 0.6 Hz, 1H), 7.15 (td, J=7.6, 1.3 Hz, 1H), 7.09 (td, J=7.7, 1.4 Hz, 1H), 5.85 (s, 1H), 3.56 (s, 8H), 1.83 (d, J=1.0 Hz, 3H).

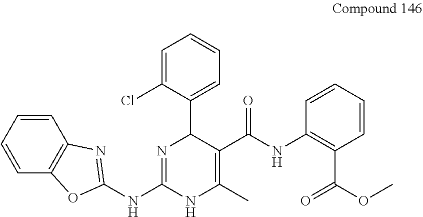 [SMILES: COC(=O)c1ccccc1NC(=O)C1=C(C)NC(Nc2nc3ccccc3o2)=NC1c1ccccc1Cl]

The title compound methyl 2-(2-(benzo[d]oxazol-2-ylamino)-4-(2-chlorophenyl)-6-methyl-1,4-dihydropyrimidine-5-carboxamido)benzoate was prepared according to Example 1 using methyl 2-aminobenzoate in Step 1 as starting material. MS m/z (M+H⁺) 516.1, ¹H NMR (400 MHz, DMSO-d₆) δ 10.72 (s, 1H), 10.38 (t, J=2.5 Hz, 1H), 10.30 (d, J=1.9 Hz, 1H), 8.16 (dd, J=8.4, 1.1 Hz, 1H), 7.87 (dd, J=8.0, 1.6 Hz, 1H), 7.56 (ddd, J=8.4, 7.3, 1.7 Hz, 1H), 7.51 (dd, J=7.6, 1.6 Hz, 1H), 7.46-7.43 (m, 1H), 7.43-7.37 (m, 3H), 7.35 (dd, J=7.3, 2.1 Hz, 1H), 7.21-7.13 (m, 2H), 7.11 (td, J=7.7, 1.3 Hz, 1H), 6.02 (dd, J=3.1, 1.1 Hz, 1H), 3.79 (s, 3H), 2.39 (d, J=0.8 Hz, 3H).

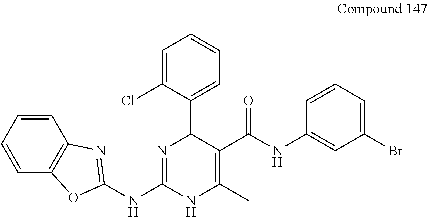 [SMILES: CC1=C(C(=O)Nc2cccc(Br)c2)C(c2ccccc2Cl)N=C(Nc2nc3ccccc3o2)N1]

The title compound 2-(benzo[d]oxazol-2-ylamino)-N-(3-bromophenyl)-4-(2-chlorophenyl)-6-methyl-1,4-dihydropyrimidine-5-carboxamide was prepared according to Example 1 using N-(3-bromophenyl)-3-oxobutanamide in Step 2 as starting material. MS m/z (M+H⁺) 536.1/538.1, ¹H NMR (400 MHz, DMSO-d₆) δ 10.30 (d, J=3.1 Hz, 1H), 10.18 (d, J=1.8 Hz, 1H), 10.05 (s, 1H), 7.88 (t, J=2.0 Hz, 1H), 7.49 (dtd, J=7.4, 4.5, 1.9 Hz, 3H), 7.42-7.33 (m, 4H), 7.26-7.21 (m, 2H), 7.18 (td, J=7.6, 1.2 Hz, 1H), 7.10 (td, J=7.7, 1.3 Hz, 1H), 6.11-6.04 (m, 1H), 2.22 (d, J=0.8 Hz, 3H).

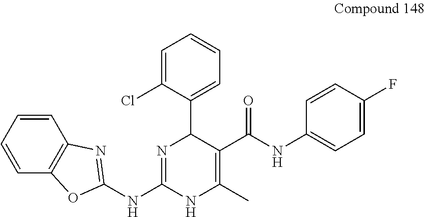 [SMILES: CC1=C(C(=O)Nc2ccc(F)cc2)C(c2ccccc2Cl)N=C(Nc2nc3ccccc3o2)N1]

The title compound 2-(benzo[d]oxazol-2-ylamino)-N-(4-fluorophenyl)-4-(2-chlorophenyl)-6-methyl-1,4-dihydropyrimidine-5-carboxamide was prepared according to Example 1 using N-(4-fluorophenyl)-3-oxobutanamide in Step 2 as starting material. MS m/z (M+H⁺) 476.1, ¹H NMR (400 MHz, DMSO-d₆) δ 10.29 (s, 1H), 10.13 (d, J=1.8 Hz, 1H), 9.95 (s, 1H), 7.58-7.47 (m, 4H), 7.42-7.33 (m, 4H), 7.17 (td, J=7.6, 1.2 Hz, 1H), 7.14-7.08 (m, 3H), 6.08 (dd, J=2.9, 1.1 Hz, 1H), 2.22 (d, J=0.9 Hz, 3H).

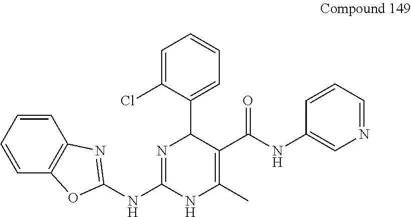 [SMILES: CC1=C(C(=O)Nc2cccnc2)C(c2ccccc2Cl)N=C(Nc2nc3ccccc3o2)N1]

The title compound 2-(benzo[d]oxazol-2-ylamino)-N-(pyridin-3-yl)-4-(2-chlorophenyl)-6-methyl-1,4-dihydropyrimidine-5-carboxamide was prepared according to Example 1 using N-(pyridin-3-yl)-3-oxobutanamide in Step 2 as starting material. MS m/z (M+H⁺) 459.1, ¹H NMR (400 MHz, DMSO-d₆) δ 10.33 (d, J=2.9 Hz, 1H), 10.32 (s, 1H), 10.25 (s, 1H), 8.86 (d, J=2.5 Hz, 1H), 8.35 (d, J=5.0 Hz, 1H), 8.13 (d, J=8.4 Hz, 1H), 7.58-7.46 (m, 3H), 7.38 (dtd, J=20.4, 9.3, 8.5, 5.6 Hz, 4H), 7.18 (t, J=7.6 Hz, 1H), 7.15-7.06 (m, 1H), 6.10 (d, J=2.9 Hz, 1H), 2.26 (s, 3H).

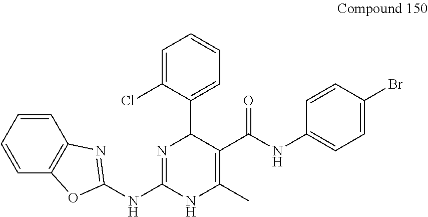 [SMILES: CC1=C(C(=O)Nc2ccc(Br)cc2)C(c2ccccc2Cl)N=C(Nc2nc3ccccc3o2)N1]

The title compound 2-(benzo[d]oxazol-2-ylamino)-N-(4-bromophenyl)-4-(2-chlorophenyl)-6-methyl-1,4-dihydropyrimidine-5-carboxamide was prepared according to Example 1 using N-(4-bromophenyl)-3-oxobutanamide in Step 2 as starting material. MS m/z (M+H⁺) 536.0/538.0, ¹H NMR (400 MHz, DMSO-d₆) δ 10.29 (t, J=2.4 Hz, 1H), 10.15 (d, J=1.8 Hz, 1H), 10.02 (s, 1H), 7.54-7.43 (m, 6H), 7.42-7.36 (m, 3H), 7.36-7.32 (m, 1H), 7.17 (td, J=7.6, 1.2 Hz, 1H), 7.13-7.07 (m, 1H), 6.08 (dd, J=3.0, 1.1 Hz, 1H), 2.22 (d, J=0.9 Hz, 3H).

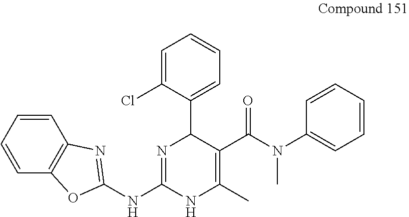 [SMILES: CC1=C(C(=O)N(C)c2ccccc2)C(c2ccccc2Cl)N=C(Nc2nc3ccccc3o2)N1]

The title compound 2-(benzo[d]oxazol-2-ylamino)-4-(2-chlorophenyl)-N,6-dimethyl-N-phenyl-1,4-dihydropyrimidine-5-carboxamide was prepared according to Example 1 using N-methyl-3-oxo-N-phenylbutanamide in Step 2 as starting material. MS m/z (M+H⁺) 472.2, ¹H NMR (400 MHz, DMSO-d₆) δ 9.94 (s, 1H), 9.76 (s, 1H), 7.50-7.45 (m, 1H), 7.45-7.33 (m, 6H), 7.32-7.24 (m, 4H), 7.14 (t, J=7.6 Hz, 1H), 7.07 (td, J=7.7, 1.4 Hz, 1H), 5.41 (s, 1H), 3.16 (s, 3H), 1.94 (s, 3H).

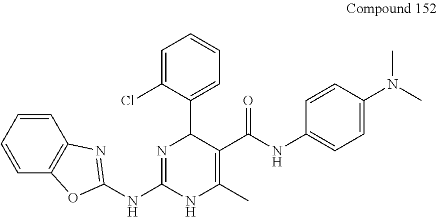 [SMILES: CC1=C(C(=O)Nc2ccc(N(C)C)cc2)C(c2ccccc2Cl)N=C(Nc2nc3ccccc3o2)N1]

The title compound 2-(benzo[d]oxazol-2-ylamino)-4-(2-chlorophenyl)-N-(4-(dimethylamino)phenyl)-6-methyl-1,4-dihydropyrimidine-5-carboxamide was prepared according to Example 1 using N-(4-(dimethylamino)phenyl)-3-oxobutanamide in Step 2 as starting material. MS m/z (M+H⁺) 501.2, ¹H NMR (400 MHz, DMSO-d₆) δ 10.35-10.20 (m, 1H), 10.11 (d, J=1.8 Hz, 1H), 9.80 (s, 1H), 7.53 (dd, J=7.6, 1.9 Hz, 1H), 7.51-7.43 (m, 3H), 7.42-7.31 (m, 5H), 7.17 (td, J=7.6, 1.2 Hz, 1H), 7.13-7.07 (m, 1H), 6.96 (s, 1H), 6.07 (dd, J=3.0, 1.2 Hz, 1H), 2.93 (s, 6H), 2.21 (d, J=0.9 Hz, 3H).

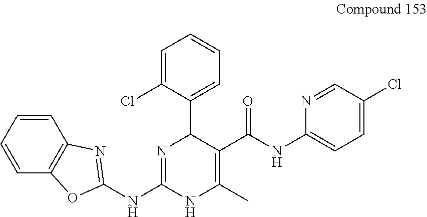 [SMILES: CC1=C(C(=O)Nc2ccc(Cl)cn2)C(c2ccccc2Cl)N=C(Nc2nc3ccccc3o2)N1]

The title compound 2-(benzo[d]oxazol-2-ylamino)-4-(2-chlorophenyl)-N-(5-chloropyridin-2-yl)-6-methyl-1,4-dihydropyrimidine-5-carboxamide was prepared according to Example 1 using N-(5-chloropyridin-2-yl)-3-oxobutanamide in Step 2 as starting material. MS m/z (M+H⁺) 493.1, ¹H NMR (400 MHz, DMSO-d₆) δ 10.60 (s, 1H), 10.30 (s, 1H), 10.18 (s, 1H), 8.36 (d, J=2.5 Hz, 1H), 7.97 (d, J=8.9 Hz, 1H), 7.84 (dd, J=8.9, 2.6 Hz, 1H), 7.58 (dd, J=7.6, 1.9 Hz, 1H), 7.49 (dd, J=7.7, 1.6 Hz, 1H), 7.44-7.29 (m, 4H), 7.17 (t, J=7.6 Hz, 1H), 7.14-7.07 (m, 1H), 6.22-6.08 (m, 1H), 2.25 (s, 3H).

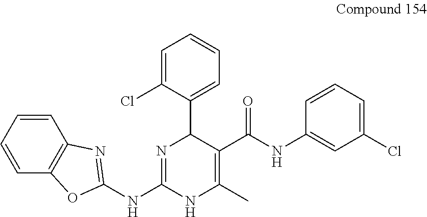 [SMILES: CC1=C(C(=O)Nc2cccc(Cl)c2)C(c2ccccc2Cl)N=C(Nc2nc3ccccc3o2)N1]

The title compound 2-(benzo[d]oxazol-2-ylamino)-N-(3-chlorophenyl)-4-(2-chlorophenyl)-6-methyl-1,4-dihydropyrimidine-5-carboxamide was prepared according to Example 1 using N-(3-chlorophenyl)-3-oxobutanamide in Step 2 as starting material. MS m/z (M+H⁺) 492.1, ¹H NMR (400 MHz, DMSO-d₆) δ 10.30 (d, J=2.8 Hz, 1H), 10.17 (d, J=1.9 Hz, 1H), 10.07 (s, 1H), 7.74 (t, J=2.0 Hz, 1H), 7.50 (ddd, J=7.4, 5.1, 1.7 Hz, 2H), 7.44 (ddd, J=8.3, 2.0, 1.0 Hz, 1H), 7.42-7.33 (m, 4H), 7.31 (t, J=8.1 Hz, 1H), 7.18 (td, J=7.6, 1.2 Hz, 1H), 7.13-7.07 (m, 2H), 6.08 (dd, J=3.0, 1.1 Hz, 1H), 2.23 (d, J=0.8 Hz, 3H).

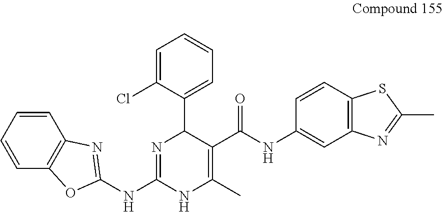 [SMILES: CC1=C(C(=O)Nc2ccc3sc(C)nc3c2)C(c2ccccc2Cl)N=C(Nc2nc3ccccc3o2)N1]

The title compound 2-(benzo[d]oxazol-2-ylamino)-4-(2-chlorophenyl)-6-methyl-N-(2-methylbenzo[d]thiazol-6-yl)-1,4-dihydropyrimidine-5-carboxamide was prepared according to Example 1 using N-(2-methylbenzo[d]thiazol-6-yl)-3-oxobutanamide in Step 2 as starting material. MS m/z (M+H⁺) 529.1, ¹H NMR (400 MHz, DMSO-d₆) δ 10.31 (s, 1H), 10.16 (s, 1H), 10.11 (s, 1H), 8.33 (d, J=2.0 Hz, 1H), 7.80 (d, J=8.8 Hz, 1H), 7.58-7.46 (m, 3H), 7.44-7.30 (m, 4H), 7.17 (t, J=7.6 Hz, 1H), 7.10 (t, J=7.7 Hz, 1H), 6.19-6.01 (m, 1H), 2.75 (s, 3H), 2.25 (s, 3H).

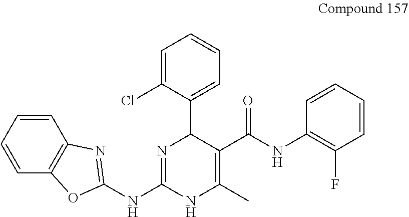 [SMILES: CC1=C(C(=O)Nc2ccccc2F)C(c2ccccc2Cl)N=C(Nc2nc3ccccc3o2)N1]

The title compound 2-(benzo[d]oxazol-2-ylamino)-N-(2-fluorophenyl)-4-(2-chlorophenyl)-6-methyl-1,4-dihydropyrimidine-5-carboxamide was prepared according to Example 1 using N-(2-fluorophenyl)-3-oxobutanamide in Step 2 as starting material. MS m/z (M+H⁺) 476.1, ¹H NMR (400 MHz, DMSO-d₆) δ 10.32-10.26 (m, 1H), 10.15 (d, J=1.8 Hz, 1H), 9.65 (s, 1H), 7.57-7.48 (m, 3H), 7.42-7.34 (m, 4H), 7.25-7.07 (m, 5H), 6.23-5.96 (m, 1H), 2.28 (d, J=0.8 Hz, 3H).

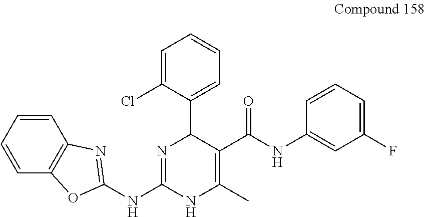 [SMILES: CC1=C(C(=O)Nc2cccc(F)c2)C(c2ccccc2Cl)N=C(Nc2nc3ccccc3o2)N1]

The title compound 2-(benzo[d]oxazol-2-ylamino)-N-(3-fluorophenyl)-4-(2-chlorophenyl)-6-methyl-1,4-dihydropyrimidine-5-carboxamide was prepared according to Example 1 using N-(3-fluorophenyl)-3-oxobutanamide in Step 2 as starting material. MS m/z (M+H⁺) 476.1, ¹H NMR (400 MHz, DMSO-d₆) δ 10.31 (d, J=2.9 Hz, 1H), 10.21-10.13 (m, 1H), 10.10 (s, 1H), 7.51 (qd, J=7.4, 7.0, 1.7 Hz, 3H), 7.43-7.28 (m, 6H), 7.17 (td, J=7.6, 1.2 Hz, 1H), 7.10 (td, J=7.7, 1.3 Hz, 1H), 6.89-6.82 (m, 1H), 6.14-6.02 (m, 1H), 2.22 (d, J=0.8 Hz, 3H).

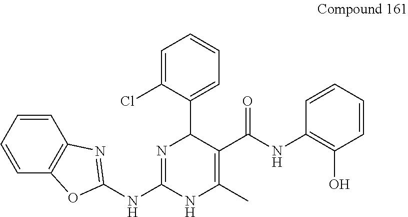 [SMILES: CC1=C(C(=O)Nc2ccccc2O)C(c2ccccc2Cl)N=C(Nc2nc3ccccc3o2)N1]

The title compound 2-(benzo[d]oxazol-2-ylamino)-N-(2-hydroxyphenyl)-4-(2-chlorophenyl)-6-methyl-1,4-dihydropyrimidine-5-carboxamide was prepared according to Example 1 using 2-aminophenol in Step 1 as starting material. MS m/z (M+H⁺) 474.1, ¹H NMR (400 MHz, DMSO-d₆) δ 10.35-10.26 (m, 1H), 10.19-10.11 (m, 1H), 9.74 (s, 1H), 8.90 (s, 1H), 7.62 (dd, J=8.0, 1.6 Hz, 1H), 7.59-7.49 (m, 2H), 7.43-7.33 (m, 4H), 7.17 (td, J=7.6, 1.2 Hz, 1H), 7.10 (td, J=7.7, 1.3 Hz, 1H), 6.96-6.88 (m, 1H), 6.82 (dd, J=8.0, 1.4 Hz, 1H), 6.72 (td, J=7.6, 1.5 Hz, 1H), 6.07 (dd, J=3.1, 1.1 Hz, 1H), 2.34 (d, J=0.8 Hz, 3H).

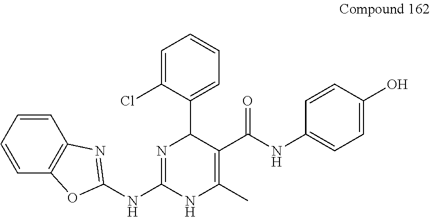 [SMILES: CC1=C(C(=O)Nc2ccc(O)cc2)C(c2ccccc2Cl)N=C(Nc2nc3ccccc3o2)N1]

The title compound 2-(benzo[d]oxazol-2-ylamino)-N-(4-hydroxyphenyl)-4-(2-chlorophenyl)-6-methyl-1,4-dihydropyrimidine-5-carboxamide was prepared according to Example 1 using 4-aminophenol in Step 1 as starting material. MS m/z (M+H⁺) 474.1, ¹H NMR (400 MHz, DMSO-d₆) δ 10.27 (s, 1H), 10.06 (s, 1H), 9.65 (s, 1H), 9.17 (s, 1H), 7.53 (dd, J=7.6, 1.9 Hz, 1H), 7.49 (dd, J=7.8, 1.6 Hz, 1H), 7.46-7.24 (m, 6H), 7.20-7.13 (m, 1H), 7.09 (td, J=7.7, 1.4 Hz, 1H), 6.71-6.60 (m, 2H), 6.05 (d, J=3.0 Hz, 1H), 2.20 (s, 3H).

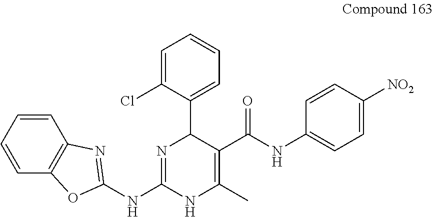 [SMILES: CC1=C(C(=O)Nc2ccc([N+](=O)[O-])cc2)C(c2ccccc2Cl)N=C(Nc2nc3ccccc3o2)N1]

The title compound 2-(benzo[d]oxazol-2-ylamino)-4-(2-chlorophenyl)-6-methyl-N-(4-nitrophenyl)-1,4-dihydropyrimidine-5-carboxamide was prepared according to Example 1 using N-(4-nitrophenyl)-3-oxobutanamide in Step 2 as starting material. MS m/z (M+H⁺) 503.1, ¹H NMR (400 MHz, DMSO-d₆) δ 10.48 (s, 1H), 10.33 (t, J=2.4 Hz, 1H), 10.26 (d, J=1.8 Hz, 1H), 8.19 (d, J=9.2 Hz, 2H), 7.84-7.77 (m, 2H), 7.49 (ddd, J=7.6, 4.1, 1.7 Hz, 2H), 7.44-7.30 (m, 4H), 7.18 (td, J=7.6, 1.3 Hz, 1H), 7.11 (td, J=7.7, 1.4 Hz, 1H), 6.17-6.05 (m, 1H), 2.25 (d, J=0.8 Hz, 3H).

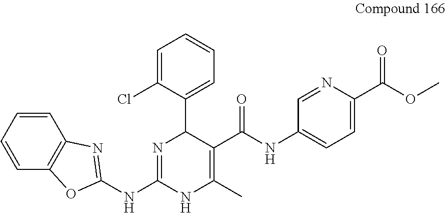 [SMILES: COC(=O)c1ccc(NC(=O)C2=C(C)NC(Nc3nc4ccccc4o3)=NC2c2ccccc2Cl)cn1]

The title compound methyl 5-(2-(benzo[d]oxazol-2-ylamino)-4-(2-chlorophenyl)-6-methyl-1,4-dihydropyrimidine-5-carboxamido)picolinate was prepared according to Example 1 using methyl 5-aminopicolinate in Step 1 as starting material. MS m/z (M+H⁺) 516.1, ¹H NMR (400 MHz, DMSO-d₆) δ 10.43 (s, 1H), 10.34 (d, J=3.0 Hz, 1H), 10.26 (s, 1H), 8.82 (d, J=2.5 Hz, 1H), 8.23-8.13 (m, 1H), 8.01 (d, J=8.6 Hz, 1H), 7.50 (ddd, J=7.7, 3.5, 1.6 Hz, 2H), 7.46-7.28 (m, 4H), 7.18 (tt, J=7.7, 0.9 Hz, 1H), 7.11 (td, J=7.6, 1.2 Hz, 1H), 6.11 (d, J=3.0 Hz, 1H), 3.84 (d, J=0.8 Hz, 3H), 2.27 (s, 3H).

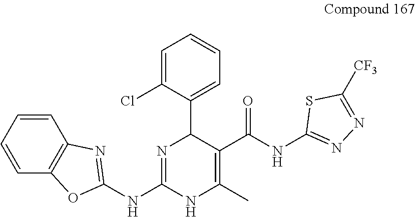 [SMILES: CC1=C(C(=O)Nc2nnc(C(F)(F)F)s2)C(c2ccccc2Cl)N=C(Nc2nc3ccccc3o2)N1]

The title compound 2-(benzo[d]oxazol-2-ylamino)-4-(2-chlorophenyl)-6-methyl-N-(5-(trifluoromethyl)-1,3,4-thiadiazol-2-yl)-1,4-dihydropyrimidine-5-carboxamide was prepared according to Example 1 using 5-(trifluoromethyl)-1,3,4-thiadiazol-2-amine in Step 1 as starting material. MS m/z (M+H⁺) 534.0, ¹H NMR (400 MHz, DMSO-d₆) δ 13.15 (s, 1H), 10.52 (d, J=1.8 Hz, 1H), 10.46 (d, J=2.6 Hz, 1H), 7.51 (dd, J=6.8, 2.2 Hz, 1H), 7.45-7.38 (m, 3H), 7.35 (ddd, J=6.8, 4.2, 2.0 Hz, 2H), 7.23-7.17 (m, 1H), 7.13 (td, J=7.4, 1.2 Hz, 1H), 6.25 (d, J=3.2 Hz, 1H), 2.37 (s, 3H).

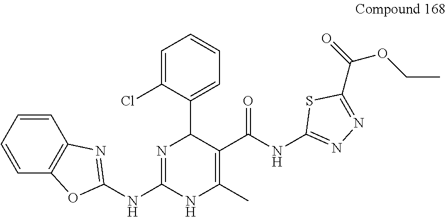 [SMILES: CCOC(=O)c1nnc(NC(=O)C2=C(C)NC(Nc3nc4ccccc4o3)=NC2c2ccccc2Cl)s1]

The title compound ethyl 5-(2-(benzo[d]oxazol-2-ylamino)-4-(2-chlorophenyl)-6-methyl-1,4-dihydropyrimidine-5-carboxamido)-1,3,4-thiadiazole-2-carboxylate was prepared according to Example 1 using ethyl 5-amino-1,3,4-thiadiazole-2-carboxylate in Step 1 as starting material. MS m/z (M+H⁺) 538.1, ¹H NMR (400 MHz, DMSO-d₆) δ 12.92 (s, 1H), 10.48 (s, 1H), 10.44 (d, J=2.7 Hz, 1H), 7.51 (dd, J=7.0, 2.1 Hz, 1H), 7.44-7.39 (m, 3H), 7.35 (ddd, J=7.1, 4.8, 2.0 Hz, 2H), 7.19 (tt, J=7.7, 1.0 Hz, 1H), 7.12 (td, J=7.4, 1.2 Hz, 1H), 6.24 (d, J=3.1 Hz, 1H), 4.38 (q, J=7.1 Hz, 2H), 2.36 (s, 3H), 1.32 (td, J=7.1, 0.7 Hz, 3H).

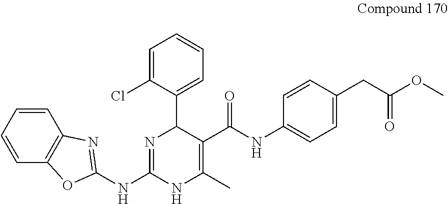 [SMILES: COC(=O)Cc1ccc(NC(=O)C2=C(C)NC(Nc3nc4ccccc4o3)=NC2c2ccccc2Cl)cc1]

The title compound methyl 2-(4-(2-(benzo[d]oxazol-2-ylamino)-4-(2-chlorophenyl)-6-methyl-1,4-dihydropyrimidine-5-carboxamido)phenyl)acetate was prepared according to Example 1 using methyl 2-(4-aminophenyl)acetate in Step 1 as starting material. MS m/z (M+H⁺) 530.1, ¹H NMR (400 MHz, DMSO-d₆) δ 10.30 (d, J=2.7 Hz, 1H), 10.12 (s, 1H), 9.91 (s, 1H), 7.53 (dd, J=7.6, 1.9 Hz, 1H), 7.49 (dt, J=5.4, 1.5 Hz, 2H), 7.45-7.31 (m, 5H), 7.25-7.15 (m, 2H), 7.10 (td, J=7.5, 1.3 Hz, 1H), 6.93 (dd, J=7.6, 1.6 Hz, 1H), 6.08 (d, J=2.9 Hz, 1H), 3.61 (s, 2H), 3.60 (d, J=0.6 Hz, 3H), 2.22 (s, 3H).

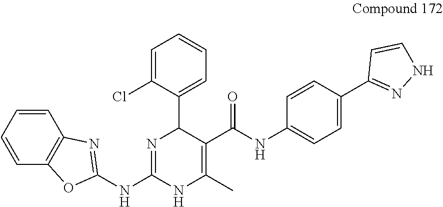 [SMILES: CC1=C(C(=O)Nc2ccc(-c3cc[nH]n3)cc2)C(c2ccccc2Cl)N=C(Nc2nc3ccccc3o2)N1]

The title compound N-(3-(1H-pyrazol-3-yl)phenyl)-2-(benzo[d]oxazol-2-ylamino)-4-(2-chlorophenyl)-6-methyl-1,4-dihydropyrimidine-5-carboxamide was prepared according to Example 1 using 3-(1H-pyrazol-3-yl)aniline in Step 1 as starting material. MS m/z (M+H⁺) 524.1

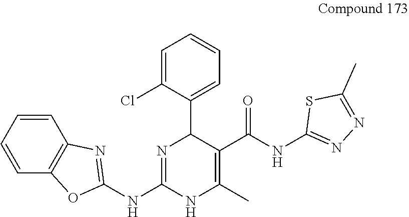 [SMILES: CC1=C(C(=O)Nc2nnc(C)s2)C(c2ccccc2Cl)N=C(Nc2nc3ccccc3o2)N1]

The title compound 2-(benzo[d]oxazol-2-ylamino)-4-(2-chlorophenyl)-6-methyl-N-(5-methyl-1,3,4-thiadiazol-2-yl)-1,4-dihydropyrimidine-5-carboxamide was prepared according to Example 1 using 5-methyl-1,3,4-thiadiazol-2-amine in Step 1 as starting material. MS m/z (M+H⁺) 480.1, ¹H NMR (400 MHz, DMSO-d₆) δ 12.31 (s, 1H), 10.50-10.19 (m, 2H), 7.50 (dd, J=7.1, 1.9 Hz, 1H), 7.47-7.30 (m, 5H), 7.22-7.15 (m, 1H), 7.11 (td, J=7.6, 1.3 Hz, 1H), 6.21 (d, J=3.0 Hz, 1H), 2.56 (s, 3H), 2.31 (d, J=11.7 Hz, 3H).

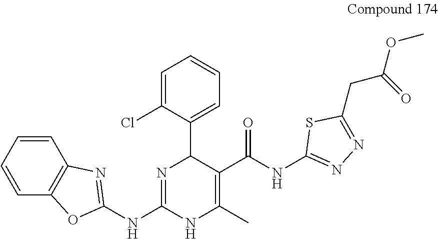 [SMILES: COC(=O)Cc1nnc(NC(=O)C2=C(C)NC(Nc3nc4ccccc4o3)=NC2c2ccccc2Cl)s1]

The title compound methyl 2-(5-(2-(benzo[d]oxazol-2-ylamino)-4-(2-chlorophenyl)-6-methyl-1,4-dihydropyrimidine-5-carboxamido)-1,3,4-thiadiazol-2-yl)acetate was prepared according to Example 1 using methyl 2-(5-amino-1,3,4-thiadiazol-2-yl)acetate in Step 1 as starting material. MS m/z (M+H⁺) 538.1

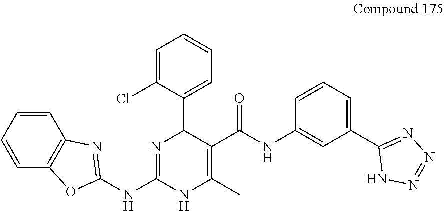 [SMILES: CC1=C(C(=O)Nc2cccc(-c3nnn[nH]3)c2)C(c2ccccc2Cl)N=C(Nc2nc3ccccc3o2)N1]

The title compound N-(3-(1H-tetrazol-5-yl)phenyl)-2-(benzo[d]oxazol-2-ylamino)-4-(2-chlorophenyl)-6-methyl-1,4-dihydropyrimidine-5-carboxamide was prepared according to Example 1 using 3-(1H-tetrazol-5-yl)aniline in Step 1 as starting material. MS m/z (M+H⁺) 526.1

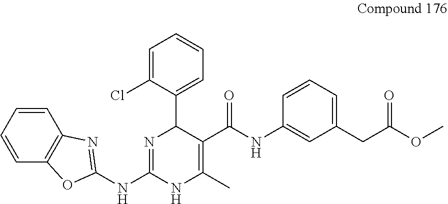 [SMILES: COC(=O)Cc1cccc(NC(=O)C2=C(C)NC(Nc3nc4ccccc4o3)=NC2c2ccccc2Cl)c1]

The title compound methyl 2-(3-(2-(benzo[d]oxazol-2-ylamino)-4-(2-chlorophenyl)-6-methyl-1,4-dihydropyrimidine-5-carboxamido)phenyl)acetate was prepared according to Example 1 using methyl 2-(3-aminophenyl)acetate in Step 1 as starting material. MS m/z (M+H⁺) 530.1

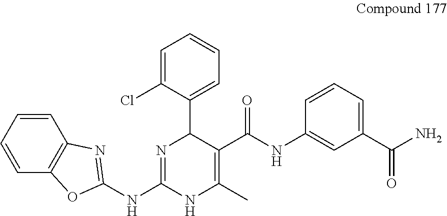 [SMILES: CC1=C(C(=O)Nc2cccc(C(N)=O)c2)C(c2ccccc2Cl)N=C(Nc2nc3ccccc3o2)N1]

The title compound 2-(benzo[d]oxazol-2-ylamino)-N-(3-carbamoylphenyl)-4-(2-chlorophenyl)-6-methyl-1,4-dihydropyrimidine-5-carboxamide was prepared according to Example 1 using 3-aminobenzamide in Step 1 as starting material. MS m/z (M+H⁺) 501.1, ¹H NMR (400 MHz, DMSO-d₆) δ 10.31 (s, 1H), 10.13 (d, J=1.7 Hz, 1H), 10.01 (s, 1H), 8.01 (d, J=2.0 Hz, 1H), 7.90 (s, 1H), 7.71 (dd, J=8.1, 2.2 Hz, 1H), 7.56-7.47 (m, 3H), 7.42-7.29 (m, 6H), 7.17 (td, J=7.7, 1.2 Hz, 1H), 7.13-7.07 (m, 1H), 6.10 (d, J=2.9 Hz, 1H), 2.25 (d, J=0.9 Hz, 3H).

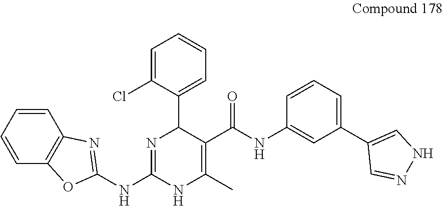 [SMILES: CC1=C(C(=O)Nc2cccc(-c3cn[nH]c3)c2)C(c2ccccc2Cl)N=C(Nc2nc3ccccc3o2)N1]

The title compound N-(3-(1H-pyrazol-4-yl)phenyl)-2-(benzo[d]oxazol-2-ylamino)-4-(2-chlorophenyl)-6-methyl-1,4-dihydropyrimidine-5-carboxamide was prepared according to Example 1 using 3-(1H-pyrazol-4-yl)aniline in Step 1 as starting material. MS m/z (M+H⁺) 524.1

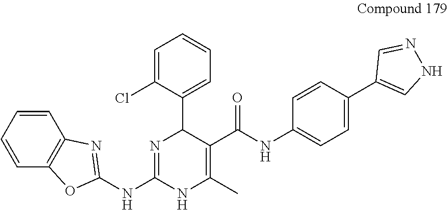 [SMILES: CC1=C(C(=O)Nc2ccc(-c3cn[nH]c3)cc2)C(c2ccccc2Cl)N=C(Nc2nc3ccccc3o2)N1]

The title compound N-(4-(1H-pyrazol-4-yl)phenyl)-2-(benzo[d]oxazol-2-ylamino)-4-(2-chlorophenyl)-6-methyl-1,4-dihydropyrimidine-5-carboxamide was prepared according to Example 1 using 4-(1H-pyrazol-4-yl)aniline in Step 1 as starting material. MS m/z (M+H⁺) 524.1

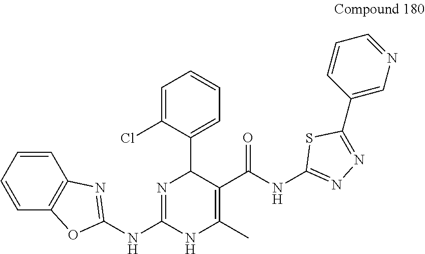 [SMILES: CC1=C(C(=O)Nc2nnc(-c3cccnc3)s2)C(c2ccccc2Cl)N=C(Nc2nc3ccccc3o2)N1]

The title compound 2-(benzo[d]oxazol-2-ylamino)-4-(2-chlorophenyl)-6-methyl-N-(5-(pyridin-3-yl)-1,3,4-thiadiazol-2-yl)-1,4-dihydropyrimidine-5-carboxamide was prepared according to Example 1 using 5-(pyridin-3-yl)-1,3,4-thiadiazol-2-amine in Step 1 as starting material. MS m/z (M+H⁺) 542.7, ¹H NMR (400 MHz, DMSO-d₆) δ 10.49-10.37 (m, 2H), 8.74 (dt, J=4.5, 1.3 Hz, 2H), 7.99-7.88 (m, 2H), 7.51 (dt, J=7.7, 1.4 Hz, 1H), 7.48-7.29 (m, 6H), 7.20 (tt, J=7.6, 1.2 Hz, 1H), 7.16-7.07 (m, 1H), 6.26 (d, J=3.0 Hz, 1H), 2.37 (s, 3H).

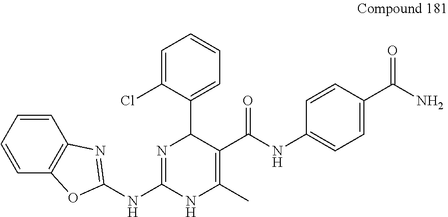 [SMILES: CC1=C(C(=O)Nc2ccc(C(N)=O)cc2)C(c2ccccc2Cl)N=C(Nc2nc3ccccc3o2)N1]

The title compound 2-(benzo[d]oxazol-2-ylamino)-N-(4-carbamoylphenyl)-4-(2-chlorophenyl)-6-methyl-1,4-dihydropyrimidine-5-carboxamide was prepared according to Example 1 using 4-aminobenzamide in Step 1 as starting material. MS m/z (M+H⁺) 501.1, ¹H NMR (400 MHz, DMSO-d₆) δ 10.31 (d, J=2.9 Hz, 1H), 10.17 (s, 1H), 10.09 (s, 1H), 7.86-7.74 (m, 2H), 7.67-7.56 (m, 2H), 7.51 (ddt, J=12.8, 7.7, 1.4 Hz, 2H), 7.44-7.29 (m, 5H), 7.25-7.14 (m, 2H), 7.10 (tt, J=7.6, 1.2 Hz, 1H), 6.10 (d, J=2.9 Hz, 1H), 2.23 (s, 3H).

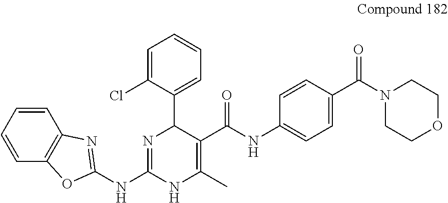 [SMILES: CC1=C(C(=O)Nc2ccc(C(=O)N3CCOCC3)cc2)C(c2ccccc2Cl)N=C(Nc2nc3ccccc3o2)N1]

The title compound 2-(benzo[d]oxazol-2-ylamino)-4-(2-chlorophenyl)-6-methyl-N-(4-(morpholine-4-carbonyl)phenyl)-1,4-dihydropyrimidine-5-carboxamide was prepared according to Example 1 using (4-aminophenyl)(morpholino)methanone in Step 1 as starting material. MS m/z (M+H⁺) 570.7, ¹H NMR (400 MHz, DMSO-d₆) δ 10.30 (s, 1H), 10.16 (s, 1H), 10.08 (s, 1H), 7.64-7.59 (m, 2H), 7.53 (dt, J=7.6, 1.4 Hz, 1H), 7.49 (dt, J=7.7, 1.3 Hz, 1H), 7.43-7.30 (m, 6H), 7.18 (tt, J=7.7, 1.3 Hz, 1H), 7.14-7.06 (m, 1H), 6.10 (d, J=2.9 Hz, 1H), 3.61 (s, 8H), 2.23 (s, 3H).

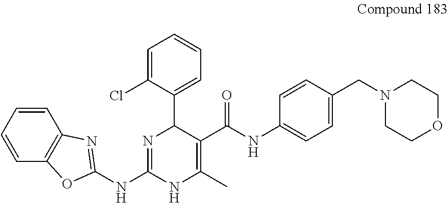 [SMILES: CC1=C(C(=O)Nc2ccc(CN3CCOCC3)cc2)C(c2ccccc2Cl)N=C(Nc2nc3ccccc3o2)N1]

The title compound 2-(benzo[d]oxazol-2-ylamino)-4-(2-chlorophenyl)-6-methyl-N-(4-(morpholinomethyl)phenyl)-1,4-dihydropyrimidine-5-carboxamide was prepared according to Example 1 using 4-(morpholinomethyl)aniline in Step 1 as starting material. MS m/z (M+H⁺) 557.1, ¹H NMR (400 MHz, DMSO-d₆) δ 10.30 (s, 1H), 10.17 (s, 1H), 10.08 (s, 1H), 7.65 (d, J=8.1 Hz, 2H), 7.57-7.45 (m, 2H), 7.43-7.29 (m, 6H), 7.22-7.13 (m, 1H), 7.13-7.04 (m, 1H), 6.10 (d, J=2.9 Hz, 1H), 4.27 (s, 2H), 3.94 (d, J=12.9 Hz, 2H), 3.59 (t, J=12.3 Hz, 2H), 3.23 (d, J=12.6 Hz, 2H), 3.13-3.00 (m, 2H), 2.22 (s, 3H).

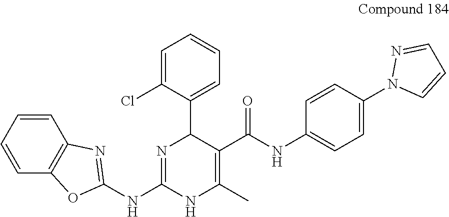 [SMILES: CC1=C(C(=O)Nc2ccc(-n3cccn3)cc2)C(c2ccccc2Cl)N=C(Nc2nc3ccccc3o2)N1]

The title compound N-(4-(1H-pyrazol-1-yl)phenyl)-2-(benzo[d]oxazol-2-ylamino)-4-(2-chlorophenyl)-6-methyl-1,4-dihydropyrimidine-5-carboxamide was prepared according to Example 1 using 4-(1H-pyrazol-1-yl)aniline in Step 1 as starting material. MS m/z (M+H⁺) 524.1, ¹H NMR (400 MHz, DMSO-d₆) δ 10.30 (d, J=2.8 Hz, 1H), 10.15 (s, 1H), 10.03 (s, 1H), 8.39 (d, J=2.5 Hz, 1H), 7.80-7.62 (m, 5H), 7.59-7.45 (m, 2H), 7.43-7.31 (m, 4H), 7.18 (tt, J=7.7, 1.2 Hz, 1H), 7.14-7.04 (m, 1H), 6.55-6.44 (m, 1H), 6.10 (d, J=2.8 Hz, 1H), 2.24 (s, 3H).

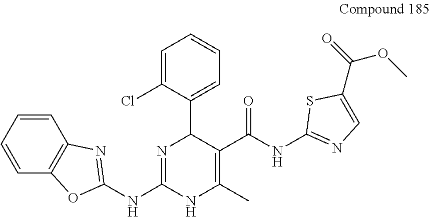 [SMILES: COC(=O)c1cnc(NC(=O)C2=C(C)NC(Nc3nc4ccccc4o3)=NC2c2ccccc2Cl)s1]

The title compound methyl 2-(2-(benzo[d]oxazol-2-ylamino)-4-(2-chlorophenyl)-6-methyl-1,4-dihydropyrimidine-5-carboxamido)thiazole-5-carboxylate was prepared according to Example 1 using methyl 2-aminothiazole-5-carboxylate in Step 1 as starting material. MS m/z (M+H⁺) 523.0, ¹H NMR (400 MHz, DMSO-d₆) δ 12.53 (s, 1H), 10.40 (s, 2H), 8.15 (d, J=1.3 Hz, 1H), 7.51 (dt, J=7.7, 1.4 Hz, 1H), 7.48-7.28 (m, 5H), 7.23-7.16 (m, 1H), 7.16-7.08 (m, 1H), 6.23 (d, J=2.8 Hz, 1H), 3.79 (d, J=1.3 Hz, 3H), 2.32 (d, J=4.4 Hz, 3H).

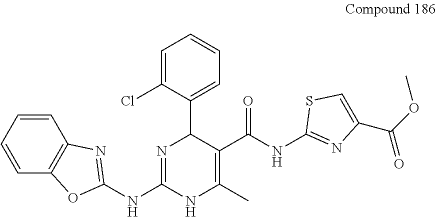 [SMILES: COC(=O)c1csc(NC(=O)C2=C(C)NC(Nc3nc4ccccc4o3)=NC2c2ccccc2Cl)n1]

The title compound methyl 2-(2-(benzo[d]oxazol-2-ylamino)-4-(2-chlorophenyl)-6-methyl-1,4-dihydropyrimidine-5-carboxamido)thiazole-4-carboxylate was prepared according to Example 1 using methyl 2-aminothiazole-4-carboxylate in Step 1 as starting material. MS m/z (M+H⁺) 522.7, ¹H NMR (400 MHz, DMSO-d₆) δ 12.45 (s, 1H), 10.37 (d, J=16.7 Hz, 2H), 8.03 (d, J=1.1 Hz, 1H), 7.51 (dt, J=7.9, 1.4 Hz, 1H), 7.48-7.31 (m, 5H), 7.19 (tt, J=7.7, 1.3 Hz, 1H), 7.11 (tt, J=7.6, 1.3 Hz, 1H), 6.22 (d, J=3.0 Hz, 1H), 3.80 (d, J=1.2 Hz, 3H), 2.31 (s, 3H).

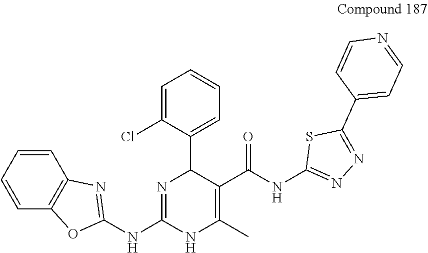 [SMILES: CC1=C(C(=O)Nc2nnc(-c3ccncc3)s2)C(c2ccccc2Cl)N=C(Nc2nc3ccccc3o2)N1]

The title compound 2-(benzo[d]oxazol-2-ylamino)-4-(2-chlorophenyl)-6-methyl-N-(5-(pyridin-4-yl)-1,3,4-thiadiazol-2-yl)-1,4-dihydropyrimidine-5-carboxamide was prepared according to Example 1 using 3-oxo-N-(5-(pyridin-4-yl)-1,3,4-thiadiazol-2-yl)butanamide in Step 1 as starting material. MS m/z (M+H⁺) 542.7

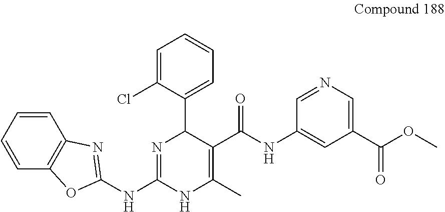 [SMILES: COC(=O)c1cncc(NC(=O)C2=C(C)NC(Nc3nc4ccccc4o3)=NC2c2ccccc2Cl)c1]

The title compound methyl 5-(2-(benzo[d]oxazol-2-ylamino)-4-(2-chlorophenyl)-6-methyl-1,4-dihydropyrimidine-5-carboxamido)nicotinate was prepared according to Example 1 using methyl 5-aminonicotinate in Step 1 as starting material. MS m/z (M+H⁺) 516.7

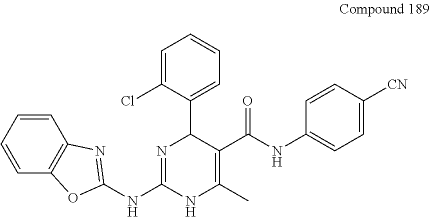 [SMILES: CC1=C(C(=O)Nc2ccc(C#N)cc2)C(c2ccccc2Cl)N=C(Nc2nc3ccccc3o2)N1]

The title compound 2-(benzo[d]oxazol-2-ylamino)-4-(2-chlorophenyl)-N-(4-cyanophenyl)-6-methyl-1,4-dihydropyrimidine-5-carboxamide was prepared according to Example 1 using 4-aminobenzonitrile in Step 1 as starting material. MS m/z (M+H⁺) 482.7

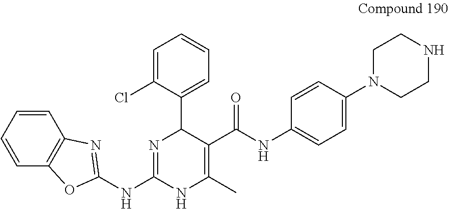 [SMILES: CC1=C(C(=O)Nc2ccc(N3CCNCC3)cc2)C(c2ccccc2Cl)N=C(Nc2nc3ccccc3o2)N1]

The title compound 2-(benzo[d]oxazol-2-ylamino)-4-(2-chlorophenyl)-6-methyl-N-(4-(piperazin-1-yl)phenyl)-1,4-dihydropyrimidine-5-carboxamide was prepared according to Example 1 using 4-(piperazin-1-yl)aniline in Step 1 as starting material. MS m/z (M+H⁺) 541.8

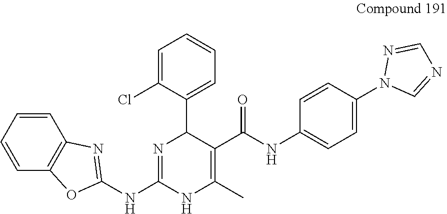 [SMILES: CC1=C(C(=O)Nc2ccc(-n3cncn3)cc2)C(c2ccccc2Cl)N=C(Nc2nc3ccccc3o2)N1]

The title compound N-(4-(1H-1,2,4-triazol-1-yl)phenyl)-2-(benzo[d]oxazol-2-ylamino)-4-(2-chlorophenyl)-6-methyl-1,4-dihydropyrimidine-5-carboxamide was prepared according to Example 1 using 4-(1H-1,2,4-triazol-1-yl)aniline in Step 1 as starting material. MS m/z (M+H⁺) 524.7

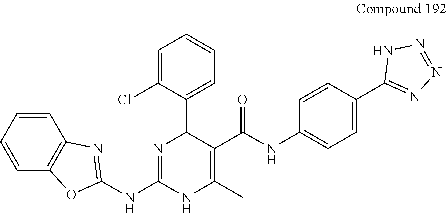 [SMILES: CC1=C(C(=O)Nc2ccc(-c3nnn[nH]3)cc2)C(c2ccccc2Cl)N=C(Nc2nc3ccccc3o2)N1]

The title compound N-(4-(1H-tetrazol-5-yl)phenyl)-2-(benzo[d]oxazol-2-ylamino)-4-(2-chlorophenyl)-6-methyl-1,4-dihydropyrimidine-5-carboxamide was prepared according to Example 1 using 4-(1H-tetrazol-5-yl)aniline in Step 1 as starting material. MS m/z (M+H⁺) 525.7

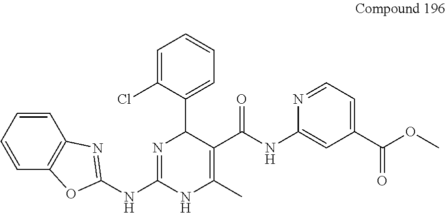 [SMILES: COC(=O)c1ccnc(NC(=O)C2=C(C)NC(Nc3nc4ccccc4o3)=NC2c2ccccc2Cl)c1]

The title compound methyl 2-(2-(benzo[d]oxazol-2-ylamino)-4-(2-chlorophenyl)-6-methyl-1,4-dihydropyrimidine-5-carboxamido)isonicotinate was prepared according to Example 1 using methyl 2-aminoisonicotinate in Step 1 as starting material. MS m/z (M+H⁺) 517.1, ¹H NMR (400 MHz, DMSO-d₆) δ 10.71 (s, 1H), 10.31 (s, 1H), 10.19 (s, 1H), 8.53-8.48 (m, 1H), 8.45 (d, J=1.2 Hz, 1H), 7.59 (dt, J=7.6, 1.4 Hz, 1H), 7.55-7.46 (m, 2H), 7.43-7.29 (m, 4H), 7.18 (tt, J=7.7, 1.2 Hz, 1H), 7.14-7.05 (m, 1H), 6.17 (d, J=2.9 Hz, 1H), 3.87 (d, J=1.1 Hz, 3H), 2.27 (s, 3H).

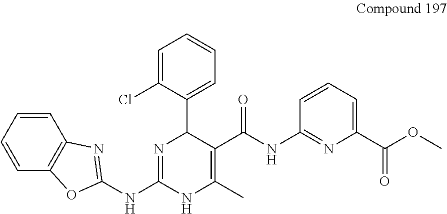 [SMILES: COC(=O)c1cccc(NC(=O)C2=C(C)NC(Nc3nc4ccccc4o3)=NC2c2ccccc2Cl)n1]

The title compound methyl 2-(2-(benzo[d]oxazol-2-ylamino)-4-(2-chlorophenyl)-6-methyl-1,4-dihydropyrimidine-5-carboxamido)isonicotinate was prepared according to Example 1 using methyl 6-aminopicolinate in Step 1 as starting material. MS m/z (M+H⁺) 517.1, ¹H NMR (400 MHz, DMSO-d₆) δ 10.87 (s, 1H), 10.28 (s, 1H), 10.14 (s, 1H), 8.15 (d, J=8.3 Hz, 1H), 7.96-7.89 (m, 1H), 7.78-7.72 (m, 1H), 7.65 (dt, J=7.6, 1.4 Hz, 1H), 7.49 (dt, J=7.7, 1.3 Hz, 1H), 7.43-7.30 (m, 4H), 7.17 (tt, J=7.6, 1.2 Hz, 1H), 7.14-7.06 (m, 1H), 6.18 (d, J=2.8 Hz, 1H), 3.87 (d, J=1.0 Hz, 3H), 2.25 (s, 3H).

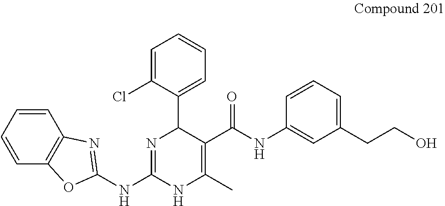 [SMILES: CC1=C(C(=O)Nc2cccc(CCO)c2)C(c2ccccc2Cl)N=C(Nc2nc3ccccc3o2)N1]

The title compound 2-(benzo[d]oxazol-2-ylamino)-4-(2-chlorophenyl)-N-(3-(2-hydroxyethyl)phenyl)-6-methyl-1,4-dihydropyrimidine-5-carboxamide was prepared according to Example 1 using 2-(3-aminophenyl)ethanol in Step 1 as starting material. MS m/z (M+H⁺) 502.1

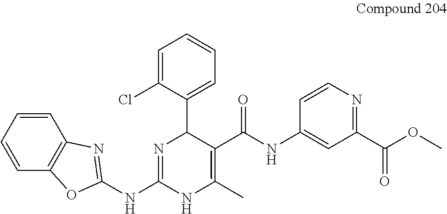 [SMILES: COC(=O)c1cc(NC(=O)C2=C(C)NC(Nc3nc4ccccc4o3)=NC2c2ccccc2Cl)ccn1]

The title compound methyl 4-(2-(benzo[d]oxazol-2-ylamino)-4-(2-chlorophenyl)-6-methyl-1,4-dihydropyrimidine-5-carboxamido)picolinate was prepared according to Example 1 using methyl 4-aminopicolinate in Step 1 as starting material. MS m/z (M+H⁺) 517.1

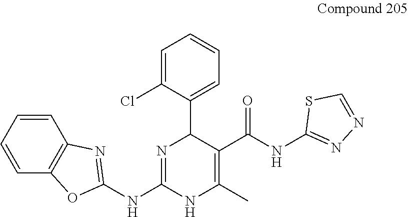 [SMILES: CC1=C(C(=O)Nc2nncs2)C(c2ccccc2Cl)N=C(Nc2nc3ccccc3o2)N1]

The title compound 2-(benzo[d]oxazol-2-ylamino)-4-(2-chlorophenyl)-6-methyl-N-(1,3,4-thiadiazol-2-yl)-1,4-dihydropyrimidine-5-carboxamide was prepared according to Example 1 using 1,3,4-thiadiazol-2-amine in Step 1 as starting material. MS m/z (M+H⁺) 466.0

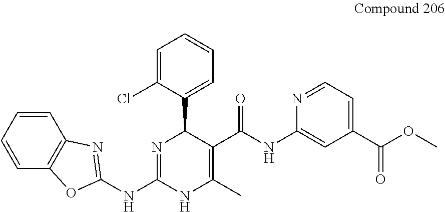 [SMILES: COC(=O)c1ccnc(NC(=O)C2=C(C)NC(Nc3nc4ccccc4o3)=N[C@H]2c2ccccc2Cl)c1]

The title compound (R)-methyl 2-(2-(benzo[d]oxazol-2-ylamino)-4-(2-chlorophenyl)-6-methyl-1,4-dihydropyrimidine-5-carboxamido)isonicotinate was prepared according to Example 1 using methyl 2-aminoisonicotinate in Step 1 as starting material followed by Chiral Separation. MS m/z (M+H⁺) 517.1

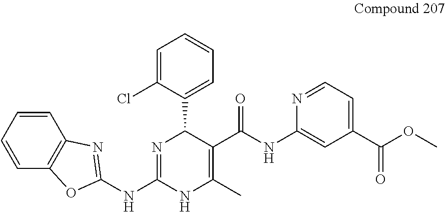 [SMILES: COC(=O)c1ccnc(NC(=O)C2=C(C)NC(Nc3nc4ccccc4o3)=N[C@@H]2c2ccccc2Cl)c1]

The title compound (S)-methyl 2-(2-(benzo[d]oxazol-2-ylamino)-4-(2-chlorophenyl)-6-methyl-1,4-dihydropyrimidine-5-carboxamido)isonicotinate was prepared according to Example 1 using methyl 2-aminoisonicotinate in Step 1 as starting material followed by Chiral Separation. MS m/z (M+H⁺) 517.1

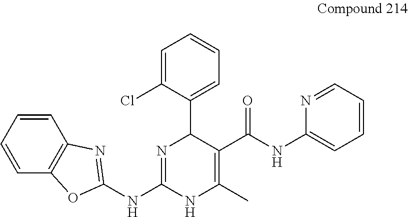 [SMILES: CC1=C(C(=O)Nc2ccccn2)C(c2ccccc2Cl)N=C(Nc2nc3ccccc3o2)N1]

The title compound 2-(benzo[d]oxazol-2-ylamino)-4-(2-chlorophenyl)-6-methyl-N-(pyridin-2-yl)-1,4-dihydropyrimidine-5-carboxamide was prepared according to Example 1 using pyridin-2-aminein in Step 1 as starting material. MS m/z (M+H⁺) 459.2, ¹H NMR (400 MHz, DMSO-d₆) δ 10.52 (s, 1H), 10.27 (t, J=2.3 Hz, 1H), 10.18 (d, J=1.8 Hz, 1H), 8.28 (ddd, J=5.0, 1.9, 0.9 Hz, 1H), 7.87 (dt, J=8.5, 1.0 Hz, 1H), 7.74 (ddd, J=8.8, 7.3, 1.9 Hz, 1H), 7.57 (dd, J=7.6, 1.9 Hz, 1H), 7.47 (dd, J=7.8, 1.5 Hz, 1H), 7.41-7.26 (m, 4H), 7.15 (td, J=7.6, 1.2 Hz, 1H), 7.11-7.05 (m, 2H), 6.12 (d, J=2.8 Hz, 1H), 2.23 (s, 3H).

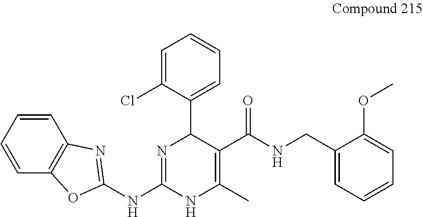 [SMILES: COc1ccccc1CNC(=O)C1=C(C)NC(Nc2nc3ccccc3o2)=NC1c1ccccc1Cl]

The title compound 2-(benzo[d]oxazol-2-ylamino)-4-(2-chlorophenyl)-N-(2-methoxybenzyl)-6-methyl-1,4-dihydropyrimidine-5-carboxamide was prepared according to Example 1 using N-(2-methoxybenzyl)-3-oxobutanamide in Step 2 as starting material. MS m/z (M+H⁺) 502.2, ¹H NMR (400 MHz, DMSO-d₆) δ 10.21 (s, 1H), 9.98 (d, J=1.9 Hz, 1H), 8.20 (t, J=5.8 Hz, 1H), 7.55-7.47 (m, 2H), 7.41-7.31 (m, 4H), 7.21-7.13 (m, 2H), 7.09 (td, J=7.7, 1.3 Hz, 1H), 6.95-6.89 (m, 1H), 6.72-6.66 (m, 2H), 6.02 (dd, J=3.0, 1.2 Hz, 1H), 4.21 (dd, J=5.8, 1.9 Hz, 2H), 3.74 (s, 3H), 2.19 (d, J=0.9 Hz, 3H).

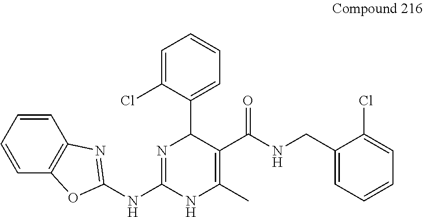 [SMILES: CC1=C(C(=O)NCc2ccccc2Cl)C(c2ccccc2Cl)N=C(Nc2nc3ccccc3o2)N1]

The title compound 2-(benzo[d]oxazol-2-ylamino)-N-(2-chlorobenzyl)-4-(2-chlorophenyl)-6-methyl-1,4-dihydropyrimidine-5-carboxamide was prepared according to Example 1 using N-(2-chlorobenzyl)-3-oxobutanamide in Step 2 as starting material. MS m/z (M+H⁺) 506.1, ¹H NMR (400 MHz, DMSO-d₆) δ 10.23 (s, 1H), 10.00 (s, 1H), 8.40 (t, J=5.8 Hz, 1H), 7.55-7.51 (m, 1H), 7.51-7.46 (m, 1H), 7.41-7.36 (m, 4H), 7.34 (ddd, J=7.7, 1.3, 0.6 Hz, 1H), 7.22 (td, J=7.7, 1.7 Hz, 1H), 7.16 (td, J=7.6, 1.2 Hz, 1H), 7.08 (tdd, J=7.5, 4.4, 1.3 Hz, 2H), 6.81 (dd, J=7.8, 1.6 Hz, 1H), 6.04 (dd, J=2.8, 1.3 Hz, 1H), 4.31 (d, J=5.7 Hz, 2H), 2.21 (d, J=0.9 Hz, 3H).

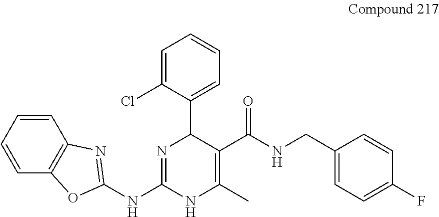 [SMILES: CC1=C(C(=O)NCc2ccc(F)cc2)C(c2ccccc2Cl)N=C(Nc2nc3ccccc3o2)N1]

The title compound 2-(benzo[d]oxazol-2-ylamino)-4-(2-chlorophenyl)-N-(4-fluorobenzyl)-6-methyl-1,4-dihydropyrimidine-5-carboxamide was prepared according to Example 1 using N-(4-fluorobenzyl)-3-oxobutanamide in Step 2 as starting material. MS m/z (M+H⁺) 490.1, ¹H NMR (400 MHz, DMSO-d₆) δ 10.18 (s, 1H), 10.00 (d, J=1.8 Hz, 1H), 8.40 (t, J=6.0 Hz, 1H), 7.54-7.50 (m, 1H), 7.47-7.44 (m, 1H), 7.40-7.32 (m, 4H), 7.17 (td, J=7.6, 1.2 Hz, 1H), 7.10 (dd, J=7.7, 1.4 Hz, 1H), 7.03-6.99 (m, 4H), 6.01 (dd, J=2.9, 1.2 Hz, 1H), 4.31-4.16 (m, 2H), 2.17 (d, J=0.9 Hz, 3H).

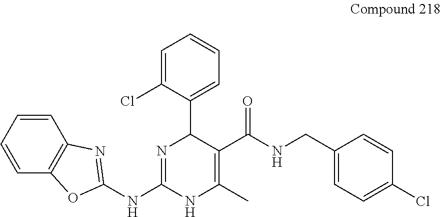 [SMILES: CC1=C(C(=O)NCc2ccc(Cl)cc2)C(c2ccccc2Cl)N=C(Nc2nc3ccccc3o2)N1]

The title compound 2-(benzo[d]oxazol-2-ylamino)-4-(2-chlorophenyl)-N-(4-chlorobenzyl)-6-methyl-1,4-dihydropyrimidine-5-carboxamide was prepared according to Example 1 using N-(4-chlorobenzyl)-3-oxobutanamide in Step 2 as starting material. MS m/z (M+H⁺) 506.1, ¹H NMR (400 MHz, DMSO-d₆) δ 10.20 (d, J=3.1 Hz, 1H), 9.99 (s, 1H), 8.41 (t, J=6.1 Hz, 1H), 7.52 (dd, J=6.9, 2.5 Hz, 1H), 7.46 (dd, J=6.3, 3.3 Hz, 1H), 7.42-7.30 (m, 4H), 7.23 (d, J=8.3 Hz, 2H), 7.16 (t, J=7.6 Hz, 1H), 7.08 (t, J=7.7 Hz, 1H), 7.00 (d, J=8.1 Hz, 2H), 6.01 (d, J=2.7 Hz, 1H), 4.36-4.14 (m, 2H), 2.18 (s, 3H).

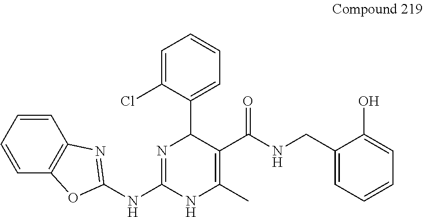 [SMILES: CC1=C(C(=O)NCc2ccccc2O)C(c2ccccc2Cl)N=C(Nc2nc3ccccc3o2)N1]

The title compound 2-(benzo[d]oxazol-2-ylamino)-4-(2-chlorophenyl)-N-(2-hydroxybenzyl)-6-methyl-1,4-dihydropyrimidine-5-carboxamide was prepared according to Example 1 using 2-(aminomethyl)phenol in Step 1 as starting material. MS m/z (M+H⁺) 488.2, ¹H NMR (400 MHz, DMSO-d₆) δ 10.22 (s, 1H), 9.99 (s, 1H), 9.47 (s, 1H), 8.27 (t, J=5.8 Hz, 1H), 7.50 (ddd, J=11.9, 6.1, 3.6 Hz, 2H), 7.37 (td, J=10.5, 9.1, 5.9 Hz, 4H), 7.16 (t, J=7.6 Hz, 1H), 7.13-7.04 (m, 1H), 7.00 (td, J=7.6, 1.8 Hz, 1H), 6.74 (d, J=8.0 Hz, 1H), 6.69-6.63 (m, 1H), 6.55 (t, J=7.4 Hz, 1H), 6.02 (d, J=2.8 Hz, 1H), 4.19 (d, J=5.8 Hz, 2H), 2.19 (s, 3H).

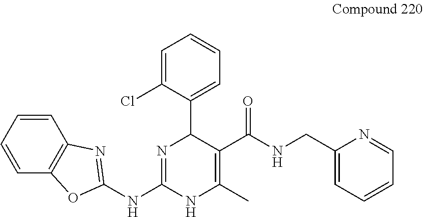 [SMILES: CC1=C(C(=O)NCc2ccccn2)C(c2ccccc2Cl)N=C(Nc2nc3ccccc3o2)N1]

The title compound 2-(benzo[d]oxazol-2-ylamino)-4-(2-chlorophenyl)-6-methyl-N-(pyridin-2-ylmethyl)-1,4-dihydropyrimidine-5-carboxamide was prepared according to Example 1 using pyridin-2-ylmethanamine in Step 1 as starting material. MS m/z (M+H⁺) 473.1, ¹H NMR (400 MHz, DMSO-d₆) δ 10.23 (s, 1H), 10.04 (d, J=1.8 Hz, 1H), 8.55-8.42 (m, 2H), 7.72 (t, J=7.7 Hz, 1H), 7.54-7.50 (m, 1H), 7.47 (dd, J=5.9, 3.6 Hz, 1H), 7.41-7.32 (m, 5H), 7.17 (td, J=7.6, 1.2 Hz, 1H), 7.09 (td, J=7.7, 1.3 Hz, 1H), 6.94 (d, J=7.9 Hz, 1H), 6.02 (dd, J=2.9, 1.2 Hz, 1H), 4.39 (d, J=5.8 Hz, 2H), 2.24 (d, J=0.9 Hz, 3H).

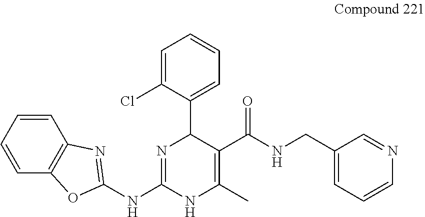 [SMILES: CC1=C(C(=O)NCc2cccnc2)C(c2ccccc2Cl)N=C(Nc2nc3ccccc3o2)N1]

The title compound 2-(benzo[d]oxazol-2-ylamino)-4-(2-chlorophenyl)-6-methyl-N-(pyridin-3-ylmethyl)-1,4-dihydropyrimidine-5-carboxamide was prepared according to Example 1 using pyridin-3-ylmethanamine in Step 1 as starting material. MS m/z (M+H⁺) 473.1, ¹H NMR (400 MHz, DMSO-d₆) δ 10.25-10.19 (m, 1H), 10.04 (d, J=1.8 Hz, 1H), 8.57 (dd, J=5.2, 1.5 Hz, 1H), 8.52-8.46 (m, 2H), 7.70 (dt, J=7.9, 1.8 Hz, 1H), 7.56-7.48 (m, 2H), 7.44-7.31 (m, 5H), 7.16 (td, J=7.6, 1.2 Hz, 1H), 7.09 (td, J=7.7, 1.3 Hz, 1H), 5.99 (dd, J=2.9, 1.2 Hz, 1H), 4.35 (t, J=5.8 Hz, 2H), 2.20 (d, J=0.9 Hz, 3H).

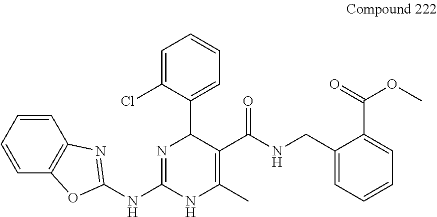 [SMILES: COC(=O)c1ccccc1CNC(=O)C1=C(C)NC(Nc2nc3ccccc3o2)=NC1c1ccccc1Cl]

The title compound methyl 2-((2-(benzo[d]oxazol-2-ylamino)-4-(2-chlorophenyl)-6-methyl-1,4-dihydropyrimidine-5-carboxamido)methyl)benzoate was prepared according to Example 1 using methyl 2-(aminomethyl)benzoate hydrochloride in Step 1 as starting material. MS m/z (M+H⁺) 530.1, ¹H NMR (400 MHz, DMSO-d₆) δ 10.22 (s, 1H), 9.99 (d, J=1.7 Hz, 1H), 8.26 (t, J=5.9 Hz, 1H), 7.86-7.75 (m, 1H), 7.56-7.51 (m, 1H), 7.51-7.46 (m, 1H), 7.41-7.36 (m, 3H), 7.36-7.28 (m, 3H), 7.16 (td, J=7.6, 1.2 Hz, 1H), 7.09 (td, J=7.7, 1.4 Hz, 1H), 6.92-6.87 (m, 1H), 6.03 (dd, J=2.8, 1.2 Hz, 1H), 4.56 (dd, J=6.0, 3.6 Hz, 2H), 3.81 (s, 3H), 2.21 (d, J=0.9 Hz, 3H).

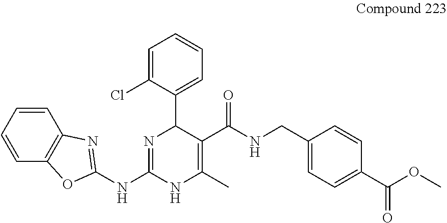 [SMILES: COC(=O)c1ccc(CNC(=O)C2=C(C)NC(Nc3nc4ccccc4o3)=NC2c2ccccc2Cl)cc1]

The title compound methyl 4-((2-(benzo[d]oxazol-2-ylamino)-4-(2-chlorophenyl)-6-methyl-1,4-dihydropyrimidine-5-carboxamido)methyl)benzoate was prepared according to Example 1 using methyl 4-(aminomethyl)benzoate hydrochloride in Step 1 as starting material. MS m/z (M+H⁺) 530.1

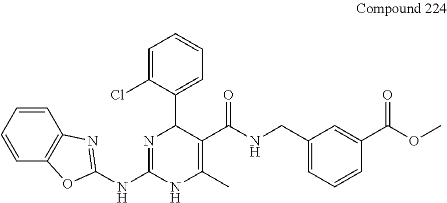 [SMILES: COC(=O)c1cccc(CNC(=O)C2=C(C)NC(Nc3nc4ccccc4o3)=NC2c2ccccc2Cl)c1]

The title compound methyl 3-((2-(benzo[d]oxazol-2-ylamino)-4-(2-chlorophenyl)-6-methyl-1,4-dihydropyrimidine-5-carboxamido)methyl)benzoate was prepared according to Example 1 using methyl 3-(aminomethyl)benzoate in Step 1 as starting material. MS m/z (M+H⁺) 530.1, ¹H NMR (400 MHz, DMSO-d₆) δ 10.22 (s, 1H), 10.01 (s, 1H), 8.50 (t, J=6.0 Hz, 1H), 7.84-7.71 (m, 2H), 7.52-7.47 (m, 1H), 7.47-7.42 (m, 1H), 7.41-7.37 (m, 1H), 7.37-7.30 (m, 4H), 7.27-7.22 (m, 1H), 7.16 (tt, J=7.7, 0.9 Hz, 1H), 7.13-7.06 (m, 1H), 6.00 (d, J=2.8 Hz, 1H), 4.32 (dd, J=5.9, 4.1 Hz, 2H), 3.84 (d, J=0.6 Hz, 3H), 2.19 (s, 3H).

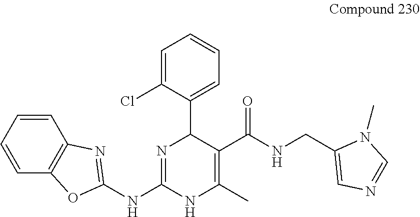 [SMILES: CC1=C(C(=O)NCc2cncn2C)C(c2ccccc2Cl)N=C(Nc2nc3ccccc3o2)N1]

The title compound 2-(benzo[d]oxazol-2-ylamino)-4-(2-chlorophenyl)-6-methyl-N-((1-methyl-1H-imidazol-5-yl)methyl)-1,4-dihydropyrimidine-5-carboxamide was prepared according to Example 1 using (1-methyl-1H-imidazol-5-yl)methanamine in Step 1 as starting material. MS m/z (M+H⁺) 476.1

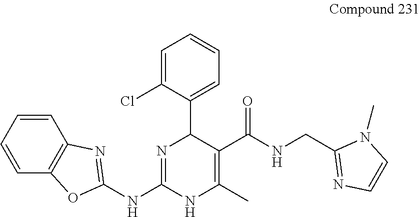 [SMILES: CC1=C(C(=O)NCc2nccn2C)C(c2ccccc2Cl)N=C(Nc2nc3ccccc3o2)N1]

The title compound 2-(benzo[d]oxazol-2-ylamino)-4-(2-chlorophenyl)-6-methyl-N-((1-methyl-1H-imidazol-2-yl)methyl)-1,4-dihydropyrimidine-5-carboxamide was prepared according to Example 1 using (1-methyl-1H-imidazol-2-yl)methanamine in Step 1 as starting material. MS m/z (M+H⁺) 476.1, ¹H NMR (400 MHz, DMSO-d₆) δ 10.29 (d, J=2.7 Hz, 1H), 10.13 (d, J=1.8 Hz, 1H), 8.48 (t, J=5.2 Hz, 1H), 7.56 (d, J=2.0 Hz, 1H), 7.54 (d, J=2.0 Hz, 1H), 7.51-7.46 (m, 1H), 7.39 (ddd, J=7.9, 1.2, 0.6 Hz, 1H), 7.35 (qd, J=2.0, 1.2 Hz, 1H), 7.34-7.31 (m, 3H), 7.17 (td, J=7.6, 1.2 Hz, 1H), 7.13-7.07 (m, 1H), 5.93 (dd, J=3.1, 1.0 Hz, 1H), 4.56-4.43 (m, 2H), 3.59 (s, 3H), 2.22 (d, J=0.8 Hz, 3H).

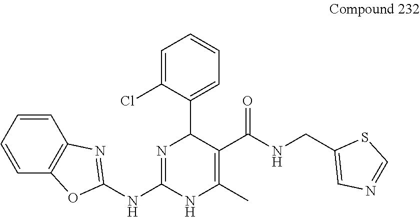 [SMILES: CC1=C(C(=O)NCc2cncs2)C(c2ccccc2Cl)N=C(Nc2nc3ccccc3o2)N1]

The title compound 2-(benzo[d]oxazol-2-ylamino)-4-(2-chlorophenyl)-6-methyl-N-(thiazol-5-ylmethyl)-1,4-dihydropyrimidine-5-carboxamide was prepared according to Example 1 using thiazol-5-ylmethanamine in Step 1 as starting material. MS m/z (M+H⁺) 479.1, ¹H NMR (400 MHz, DMSO-d₆) δ 10.22 (s, 1H), 10.00 (s, 1H), 8.89 (d, J=0.8 Hz, 1H), 8.54 (t, J=5.9 Hz, 1H), 7.61 (q, J=0.9 Hz, 1H), 7.51-7.47 (m, 1H), 7.42-7.30 (m, 5H), 7.16 (td, J=7.7, 1.2 Hz, 1H), 7.12-7.05 (m, 1H), 5.96 (dd, J=2.9, 1.1 Hz, 1H), 4.53-4.40 (m, 2H), 2.17 (d, J=0.9 Hz, 3H).

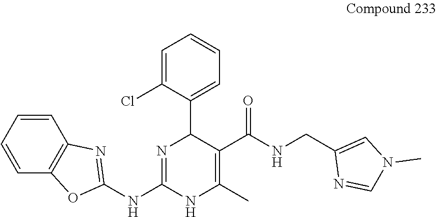 [SMILES: CC1=C(C(=O)NCc2cn(C)cn2)C(c2ccccc2Cl)N=C(Nc2nc3ccccc3o2)N1]

The title compound 2-(benzo[d]oxazol-2-ylamino)-4-(2-chlorophenyl)-6-methyl-N-((1-methyl-1H-imidazol-4-yl)methyl)-1,4-dihydropyrimidine-5-carboxamide was prepared according to Example 1 using (1-methyl-1H-imidazol-4-yl)methanamine in Step 1 as starting material. MS m/z (M+H⁺) 476.1, ¹H NMR (400 MHz, DMSO-d₆) δ 10.19 (d, J=2.7 Hz, 1H), 9.96 (s, 1H), 8.20 (t, J=5.7 Hz, 1H), 7.52-7.48 (m, 1H), 7.47-7.42 (m, 1H), 7.40-7.31 (m, 4H), 7.27 (d, J=0.9 Hz, 1H), 7.17 (td, J=7.6, 1.2 Hz, 1H), 7.12 (d, J=0.8 Hz, 1H), 7.09 (td, J=7.7, 1.3 Hz, 1H), 5.97 (dd, J=2.9, 1.1 Hz, 1H), 4.07 (d, J=5.7 Hz, 2H), 3.72 (s, 3H), 2.15 (d, J=1.0 Hz, 3H).

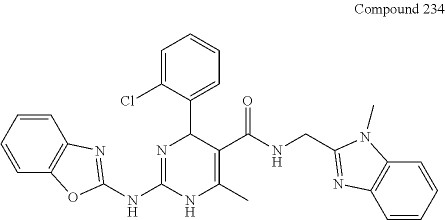 [SMILES: CC1=C(C(=O)NCc2nc3ccccc3n2C)C(c2ccccc2Cl)N=C(Nc2nc3ccccc3o2)N1]

The title compound 2-(benzo[d]oxazol-2-ylamino)-4-(2-chlorophenyl)-6-methyl-N-((1-methyl-1H-benzo[d]imidazol-2-yl)methyl)-1,4-dihydropyrimidine-5-carboxamide was prepared according to Example 1 using (1-methyl-1H-benzo[d]imidazol-2-yl)methanamine in Step 1 as starting material. MS m/z (M+H⁺) 526.1, ¹H NMR (400 MHz, DMSO-d₆) δ 10.31 (s, 1H), 10.14 (s, 1H), 8.57 (t, J=5.3 Hz, 1H), 7.83-7.71 (m, 2H), 7.57-7.48 (m, 2H), 7.48-7.33 (m, 4H), 7.33-7.26 (m, 2H), 7.18 (td, J=7.6, 1.2 Hz, 1H), 7.14-7.07 (m, 1H), 6.05-5.90 (m, 1H), 4.71 (d, J=5.3 Hz, 2H), 3.75 (s, 3H), 2.29 (d, J=0.8 Hz, 3H).

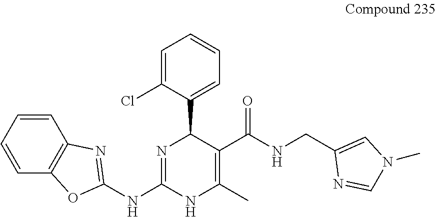 [SMILES: CC1=C(C(=O)NCc2cn(C)cn2)[C@H](c2ccccc2Cl)N=C(Nc2nc3ccccc3o2)N1]

The title compound (R)-2-(benzo[d]oxazol-2-ylamino)-4-(2-chlorophenyl)-6-methyl-N-((1-methyl-1H-imidazol-4-yl)methyl)-1,4-dihydropyrimidine-5-carboxamide was prepared according to Example 1 using (1-methyl-1H-imidazol-4-yl)methanamine in Step 1 as starting material followed by Chiral Separation. MS m/z (M+H⁺) 476.1, ¹H NMR (400 MHz, DMSO-d₆) δ 10.20 (s, 1H), 9.91 (d, J=1.8 Hz, 1H), 8.17 (t, J=5.7 Hz, 1H), 7.52-7.41 (m, 3H), 7.37-7.29 (m, 4H), 7.13 (td, J=7.6, 1.2 Hz, 1H), 7.08-7.02 (m, 1H), 6.48 (d, J=1.3 Hz, 1H), 5.95 (dd, J=2.9, 1.1 Hz, 1H), 4.07 (qdd, J=15.1, 5.7, 0.9 Hz, 2H), 3.50 (d, J=0.4 Hz, 3H), 2.16 (d, J=0.9 Hz, 3H).

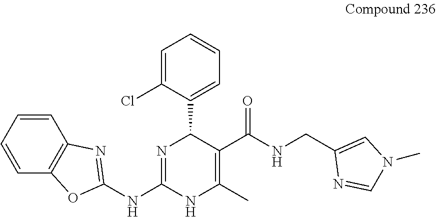 [SMILES: CC1=C(C(=O)NCc2cn(C)cn2)[C@@H](c2ccccc2Cl)N=C(Nc2nc3ccccc3o2)N1]

The title compound (S)-2-(benzo[d]oxazol-2-ylamino)-4-(2-chlorophenyl)-6-methyl-N-((1-methyl-1H-imidazol-4-yl)methyl)-1,4-dihydropyrimidine-5-carboxamide was prepared according to Example 1 using (1-methyl-1H-imidazol-4-yl)methanamine in Step 1 as starting material followed by Chiral Separation. MS m/z (M+H⁺) 476.1, ¹H NMR (400 MHz, DMSO-d₆) δ 10.19 (s, 1H), 9.91 (d, J=1.7 Hz, 1H), 8.17 (t, J=5.7 Hz, 1H), 7.51-7.41 (m, 3H), 7.37-7.29 (m, 4H), 7.13 (td, J=7.6, 1.2 Hz, 1H), 7.08-7.02 (m, 1H), 6.47 (d, J=1.3 Hz, 1H), 5.95 (dd, J=2.9, 1.1 Hz, 1H), 4.20-3.87 (m, 2H), 3.50 (d, J=0.5 Hz, 3H), 2.16 (d, J=0.9 Hz, 3H).

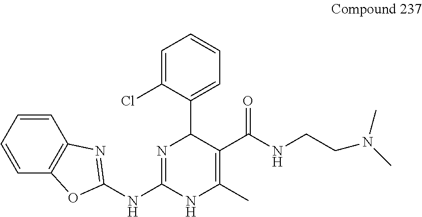 [SMILES: CC1=C(C(=O)NCCN(C)C)C(c2ccccc2Cl)N=C(Nc2nc3ccccc3o2)N1]

The title compound 2-(benzo[d]oxazol-2-ylamino)-4-(2-chlorophenyl)-N-(2-(dimethylamino)ethyl)-6-methyl-1,4-dihydropyrimidine-5-carboxamide was prepared according to Example 1 using N1,N1-dimethylethane-1,2-diamine in Step 1 as starting material. MS m/z (M+H⁺) 453.2

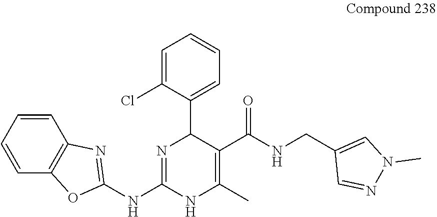 [SMILES: CC1=C(C(=O)NCc2cnn(C)c2)C(c2ccccc2Cl)N=C(Nc2nc3ccccc3o2)N1]

The title compound 2-(benzo[d]oxazol-2-ylamino)-4-(2-chlorophenyl)-6-methyl-N-((1-methyl-1H-pyrazol-4-yl)methyl)-1,4-dihydropyrimidine-5-carboxamide was prepared according to Example 1 using (1-methyl-1H-pyrazol-4-yl)methanamine in Step 1 as starting material. MS m/z (M+H⁺) 476.2

### Example 1a

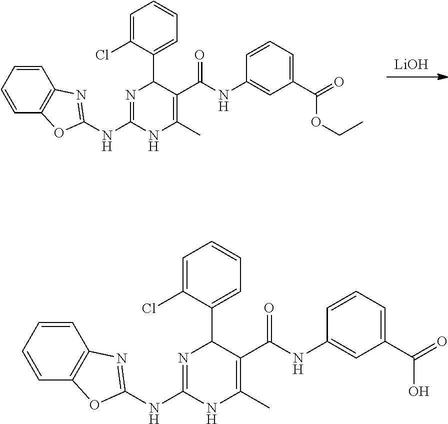 [SMILES: CCOC(=O)c1cccc(NC(=O)C2=C(C)NC(Nc3nc4ccccc4o3)=NC2c2ccccc2Cl)c1 ; "LiOH" ; CC1=C(C(=O)Nc2cccc(C(=O)O)c2)C(c2ccccc2Cl)N=C(Nc2nc3ccccc3o2)N1]

To a solution of ethyl 3-(2-(benzo[d]oxazol-2-ylamino)-4-(2-chlorophenyl)-6-methyl-1,4-dihydropyrimidine-5-carboxamido)benzoate (40 mg, 0.075 mmol) in THF (1 mL) was added LiOH (0.302 mL, 0.151 mmol) (0.5M). The mixture was stirred at R.T. for 2 days. The solvent was removed. The crude product was purified. (Compound 142) MS m/z (M+H⁺) 502.1, ¹H NMR (400 MHz, DMSO-d₆) δ 12.94 (s, 1H), 10.31 (d, J=2.8 Hz, 1H), 10.15 (d, J=1.8 Hz, 1H), 10.07 (s, 1H), 8.18 (t, J=1.8 Hz, 1H), 7.79 (ddd, J=8.2, 2.2, 1.1 Hz, 1H), 7.60 (ddd, J=7.7, 1.6, 1.1 Hz, 1H), 7.56-7.47 (m, 2H), 7.43-7.30 (m, 5H), 7.17 (td, J=7.6, 1.2 Hz, 1H), 7.13-7.07 (m, 1H), 6.10 (dd, J=2.8, 1.1 Hz, 1H), 2.24 (d, J=0.8 Hz, 3H).

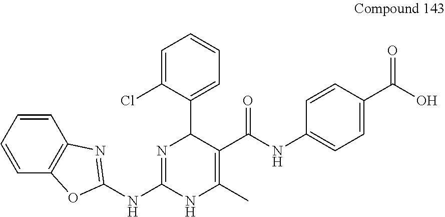 [SMILES: CC1=C(C(=O)Nc2ccc(C(=O)O)cc2)C(c2ccccc2Cl)N=C(Nc2nc3ccccc3o2)N1]

The title compound 4-(2-(benzo[d]oxazol-2-ylamino)-4-(2-chlorophenyl)-6-methyl-1,4-dihydropyrimidine-5-carboxamido)benzoic acid was prepared according to Example 1a using methyl 4-(2-(benzo[d]oxazol-2-ylamino)-4-(2-chlorophenyl)-6-methyl-1,4-dihydropyrimidine-5-carboxamido)benzoate as starting material. MS m/z (M+H⁺) 502.1, ¹H NMR (400 MHz, DMSO-d₆) δ 12.69 (s, 1H), 10.32 (s, 1H), 10.18 (d, J=2.1 Hz, 2H), 7.88-7.83 (m, 2H), 7.69-7.64 (m, 2H), 7.51 (ddd, J=11.6, 7.7, 1.6 Hz, 2H), 7.43-7.31 (m, 4H), 7.17 (td, J=7.6, 1.2 Hz, 1H), 7.14-7.07 (m, 1H), 6.19-6.04 (m, 1H), 2.24 (d, J=0.8 Hz, 3H).

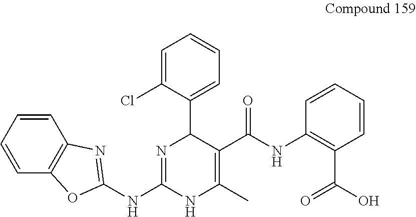 [SMILES: CC1=C(C(=O)Nc2ccccc2C(=O)O)C(c2ccccc2Cl)N=C(Nc2nc3ccccc3o2)N1]

The title compound 2-(2-(benzo[d]oxazol-2-ylamino)-4-(2-chlorophenyl)-6-methyl-1,4-dihydropyrimidine-5-carboxamido)benzoic acid was prepared according to Example 1a using methyl 2-(2-(benzo[d]oxazol-2-ylamino)-4-(2-chlorophenyl)-6-methyl-1,4-dihydropyrimidine-5-carboxamido)benzoate as starting material. MS m/z (M+H⁺) 502.1

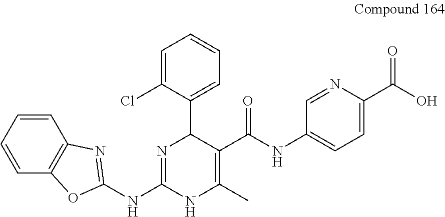 [SMILES: CC1=C(C(=O)Nc2ccc(C(=O)O)nc2)C(c2ccccc2Cl)N=C(Nc2nc3ccccc3o2)N1]

The title compound 5-(2-(benzo[d]oxazol-2-ylamino)-4-(2-chlorophenyl)-6-methyl-1,4-dihydropyrimidine-5-carboxamido)picolinic acid was prepared according to Example 1a using methyl 5-(3-oxobutanamido)picolinate as starting material. MS m/z (M+H⁺) 503.1, ¹H NMR (400 MHz, DMSO-d₆) δ 12.69 (s, 1H), 10.32 (s, 1H), 10.18 (d, J=2.1 Hz, 2H), 7.88-7.83 (m, 2H), 7.69-7.64 (m, 2H), 7.51 (ddd, J=11.6, 7.7, 1.6 Hz, 2H), 7.43-7.31 (m, 4H), 7.17 (td, J=7.6, 1.2 Hz, 1H), 7.14-7.07 (m, 1H), 6.19-6.04 (m, 1H), 2.24 (d, J=0.8 Hz, 3H).

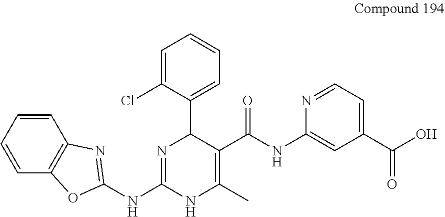 [SMILES: CC1=C(C(=O)Nc2cc(C(=O)O)ccn2)C(c2ccccc2Cl)N=C(Nc2nc3ccccc3o2)N1]

The title compound 2-(2-(benzo[d]oxazol-2-ylamino)-4-(2-chlorophenyl)-6-methyl-1,4-dihydropyrimidine-5-carboxamido)isonicotinic acid was prepared according to Example 1a using methyl 2-(2-(benzo[d]oxazol-2-ylamino)-4-(2-chlorophenyl)-6-methyl-1,4-dihydropyrimidine-5-carboxamido)isonicotinate as starting material. MS m/z (M+H⁺) 503.1, ¹H NMR (400 MHz, DMSO-d₆) δ 13.58 (s, 1H), 10.66 (s, 1H), 10.31 (s, 1H), 10.17 (d, J=1.8 Hz, 1H), 8.47 (dd, J=5.1, 0.9 Hz, 1H), 8.43 (dd, J=1.5, 0.9 Hz, 1H), 7.60 (dd, J=7.6, 1.8 Hz, 1H), 7.53-7.45 (m, 2H), 7.45-7.30 (m, 4H), 7.18 (td, J=7.6, 1.2 Hz, 1H), 7.14-7.06 (m, 1H), 6.20-6.13 (m, 1H), 2.27 (d, J=0.9 Hz, 3H).

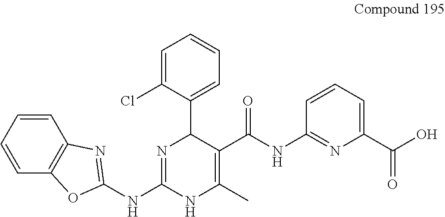 [SMILES: CC1=C(C(=O)Nc2cccc(C(=O)O)n2)C(c2ccccc2Cl)N=C(Nc2nc3ccccc3o2)N1]

The title compound 6-(2-(benzo[d]oxazol-2-ylamino)-4-(2-chlorophenyl)-6-methyl-1,4-dihydropyrimidine-5-carboxamido)picolinic acid was prepared according to Example 1a using methyl 6-(2-(benzo[d]oxazol-2-ylamino)-4-(2-chlorophenyl)-6-methyl-1,4-dihydropyrimidine-5-carboxamido)picolinate as starting material. MS m/z (M+H⁺) 503.1, 1H NMR (400 MHz, DMSO-d₆) δ 13.23 (s, 1H), 10.77 (d, J=1.6 Hz, 1H), 10.29 (s, 1H), 10.13 (s, 1H), 8.11 (dq, J=8.4, 0.9 Hz, 1H), 7.89 (td, J=8.0, 1.6 Hz, 1H), 7.72 (dq, J=7.5, 0.9 Hz, 1H), 7.64 (dt, J=7.6, 1.8 Hz, 1H), 7.49 (dt, J=7.8, 1.8 Hz, 1H), 7.43-7.29 (m, 4H), 7.17 (tt, J=7.8, 1.6 Hz, 1H), 7.10 (tt, J=7.5, 1.6 Hz, 1H), 6.18 (d, J=2.7 Hz, 1H), 2.26 (d, J=1.5 Hz, 3H).

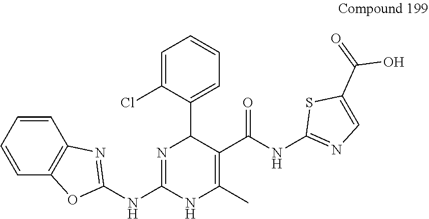 [SMILES: CC1=C(C(=O)Nc2ncc(C(=O)O)s2)C(c2ccccc2Cl)N=C(Nc2nc3ccccc3o2)N1]

The title compound 2-(2-(benzo[d]oxazol-2-ylamino)-4-(2-chlorophenyl)-6-methyl-1,4-dihydropyrimidine-5-carboxamido)thiazole-5-carboxylic acid was prepared according to Example 1a using methyl 2-(2-(benzo[d]oxazol-2-ylamino)-4-(2-chlorophenyl)-6-methyl-1,4-dihydropyrimidine-5-carboxamido)thiazole-5-carboxylate as starting material. MS m/z (M+H⁺) 509.0

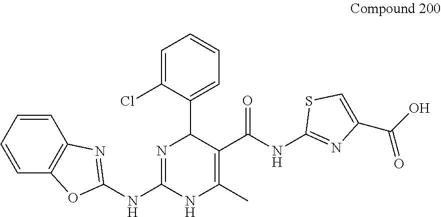 [SMILES: CC1=C(C(=O)Nc2nc(C(=O)O)cs2)C(c2ccccc2Cl)N=C(Nc2nc3ccccc3o2)N1]

The title compound 2-(2-(benzo[d]oxazol-2-ylamino)-4-(2-chlorophenyl)-6-methyl-1,4-dihydropyrimidine-5-carboxamido)thiazole-4-carboxylic acid was prepared according to Example 1a using methyl 2-(2-(benzo[d]oxazol-2-ylamino)-4-(2-chlorophenyl)-6-methyl-1,4-dihydropyrimidine-5-carboxamido)thiazole-4-carboxylate as starting material. MS m/z (M+H⁺) 509.0

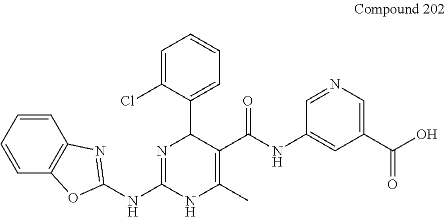 [SMILES: CC1=C(C(=O)Nc2cncc(C(=O)O)c2)C(c2ccccc2Cl)N=C(Nc2nc3ccccc3o2)N1]

The title compound 5-(2-(benzo[d]oxazol-2-ylamino)-4-(2-chlorophenyl)-6-methyl-1,4-dihydropyrimidine-5-carboxamido)nicotinic acid was prepared according to Example 1a using methyl 5-(2-(benzo[d]oxazol-2-ylamino)-4-(2-chlorophenyl)-6-methyl-1,4-dihydropyrimidine-5-carboxamido)nicotinate as starting material. MS m/z (M+H⁺) 503.1

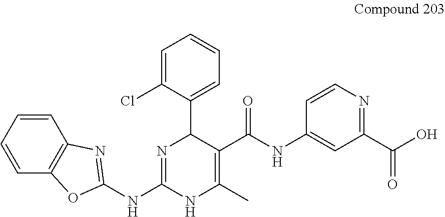 [SMILES: CC1=C(C(=O)Nc2ccnc(C(=O)O)c2)C(c2ccccc2Cl)N=C(Nc2nc3ccccc3o2)N1]

The title compound 4-(2-(benzo[d]oxazol-2-ylamino)-4-(2-chlorophenyl)-6-methyl-1,4-dihydropyrimidine-5-carboxamido)picolinic acid was prepared according to Example 1a using methyl 4-(2-(benzo[d]oxazol-2-ylamino)-4-(2-chlorophenyl)-6-methyl-1,4-dihydropyrimidine-5-carboxamido)picolinate as starting material. MS m/z (M+H⁺) 503.1

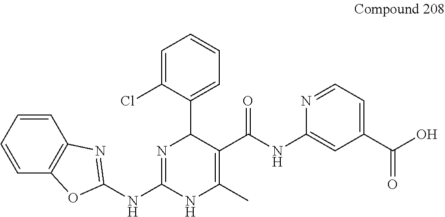 [SMILES: CC1=C(C(=O)Nc2cc(C(=O)O)ccn2)C(c2ccccc2Cl)N=C(Nc2nc3ccccc3o2)N1]

The title compound (R)-2-(2-(benzo[d]oxazol-2-ylamino)-4-(2-chlorophenyl)-6-methyl-1,4-dihydropyrimidine-5-carboxamido)isonicotinic acid was prepared according to Example 1a using (R)-methyl 2-(2-(benzo[d]oxazol-2-ylamino)-4-(2-chlorophenyl)-6-methyl-1,4-dihydropyrimidine-5-carboxamido)isonicotinate as starting material. MS m/z (M+H⁺) 503.1, ¹H NMR (400 MHz, DMSO-d₆) δ 13.59 (s, 1H), 10.66 (s, 1H), 10.30 (d, J=2.7 Hz, 1H), 10.18 (d, J=1.8 Hz, 1H), 8.47 (dd, J=5.0, 0.9 Hz, 1H), 8.43 (dd, J=1.5, 0.9 Hz, 1H), 7.60 (dd, J=7.6, 1.8 Hz, 1H), 7.53-7.47 (m, 2H), 7.43-7.30 (m, 4H), 7.18 (td, J=7.6, 1.2 Hz, 1H), 7.14-7.05 (m, 1H), 6.17 (dd, J=3.0, 1.1 Hz, 1H), 2.27 (d, J=0.9 Hz, 3H).

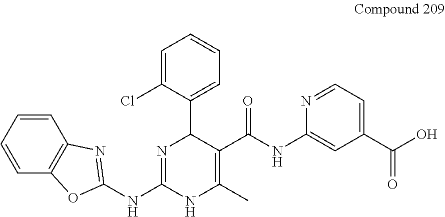 [SMILES: CC1=C(C(=O)Nc2cc(C(=O)O)ccn2)C(c2ccccc2Cl)N=C(Nc2nc3ccccc3o2)N1]

The title compound (S)-2-(2-(benzo[d]oxazol-2-ylamino)-4-(2-chlorophenyl)-6-methyl-1,4-dihydropyrimidine-5-carboxamido)isonicotinic acid was prepared according to Example 1a using (S)-methyl 2-(2-(benzo[d]oxazol-2-ylamino)-4-(2-chlorophenyl)-6-methyl-1,4-dihydropyrimidine-5-carboxamido)isonicotinate as starting material. MS m/z (M+H⁺) 503.1, ¹H NMR (400 MHz, DMSO-d₆) δ 13.59 (s, 1H), 10.66 (s, 1H), 10.31 (d, J=2.6 Hz, 1H), 10.18 (d, J=1.8 Hz, 1H), 8.47 (dd, J=5.0, 0.9 Hz, 1H), 8.43 (dd, J=1.5, 0.9 Hz, 1H), 7.60 (dd, J=7.6, 1.8 Hz, 1H), 7.53-7.47 (m, 2H), 7.43-7.29 (m, 4H), 7.18 (td, J=7.6, 1.2 Hz, 1H), 7.14-7.07 (m, 1H), 6.17 (dd, J=3.0, 1.0 Hz, 1H), 2.27 (d, J=0.9 Hz, 3H).

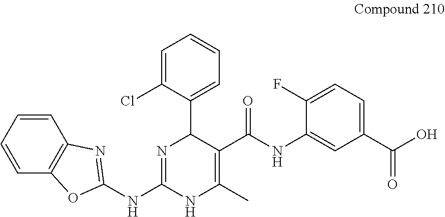 [SMILES: CC1=C(C(=O)Nc2cc(C(=O)O)ccc2F)C(c2ccccc2Cl)N=C(Nc2nc3ccccc3o2)N1]

The title compound 3-(2-(benzo[d]oxazol-2-ylamino)-4-(2-chlorophenyl)-6-methyl-1,4-dihydropyrimidine-5-carboxamido)-4-fluorobenzoic acid was prepared according to Example 1a using methyl 3-(2-(benzo[d]oxazol-2-ylamino)-4-(2-chlorophenyl)-6-methyl-1,4-dihydropyrimidine-5-carboxamido)-4-fluorobenzoate as starting material. MS m/z (M+H⁺) 520.0, ¹H NMR (400 MHz, DMSO-d₆) δ 13.02 (s, 1H), 10.29 (s, 1H), 10.15 (d, J=1.8 Hz, 1H), 9.76 (s, 1H), 8.16 (dd, J=7.5, 2.2 Hz, 1H), 7.70 (ddd, J=8.5, 4.8, 2.2 Hz, 1H), 7.50 (ddd, J=12.2, 7.7, 1.7 Hz, 2H), 7.42-7.24 (m, 5H), 7.15 (td, J=7.6, 1.2 Hz, 1H), 7.10-7.04 (m, 1H), 6.13 (s, 1H), 2.27 (s, 3H).

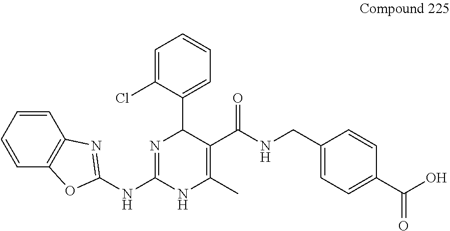 [SMILES: CC1=C(C(=O)NCc2ccc(C(=O)O)cc2)C(c2ccccc2Cl)N=C(Nc2nc3ccccc3o2)N1]

The title compound 4-((2-(benzo[d]oxazol-2-ylamino)-4-(2-chlorophenyl)-6-methyl-1,4-dihydropyrimidine-5-carboxamido)methyl)benzoic acid was prepared according to Example 1a using methyl 4-((2-(benzo[d]oxazol-2-ylamino)-4-(2-chlorophenyl)-6-methyl-1,4-dihydropyrimidine-5-carboxamido)methyl)benzoate as starting material. MS m/z (M+H⁺) 516.1, ¹H NMR (400 MHz, DMSO-d₆) δ 12.83 (s, 1H), 10.21 (s, 1H), 10.00 (s, 1H), 8.46 (t, J=5.9 Hz, 1H), 7.79-7.71 (m, 2H), 7.59-7.51 (m, 1H), 7.51-7.43 (m, 1H), 7.42-7.36 (m, 3H), 7.34 (dd, J=7.9, 1.1 Hz, 1H), 7.16 (td, J=7.6, 1.2 Hz, 1H), 7.12-7.03 (m, 3H), 6.04 (dd, J=2.9, 1.2 Hz, 1H), 4.46-4.21 (m, 2H), 2.20 (d, J=0.9 Hz, 3H).

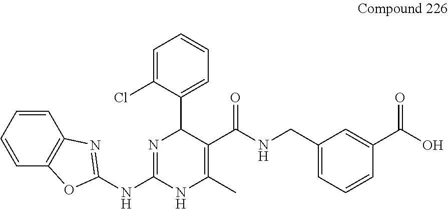 [SMILES: CC1=C(C(=O)NCc2cccc(C(=O)O)c2)C(c2ccccc2Cl)N=C(Nc2nc3ccccc3o2)N1]

The title compound 3-((2-(benzo[d]oxazol-2-ylamino)-4-(2-chlorophenyl)-6-methyl-1,4-dihydropyrimidine-5-carboxamido)methyl)benzoic acid was prepared according to Example 1a using methyl 3-((2-(benzo[d]oxazol-2-ylamino)-4-(2-chlorophenyl)-6-methyl-1,4-dihydropyrimidine-5-carboxamido)methyl)benzoate as starting material. MS m/z (M+H⁺) 516.1

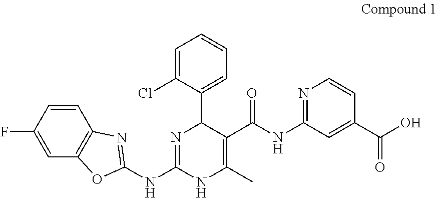 [SMILES: CC1=C(C(=O)Nc2cc(C(=O)O)ccn2)C(c2ccccc2Cl)N=C(Nc2nc3ccc(F)cc3o2)N1]

The title compound 2-(4-(2-chlorophenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 1-(6-fluorobenzo[d]oxazol-2-yl)guanidine in Step 2, Example 1a using methyl 2-(4-(2-chlorophenyl)-2-((6-fluorobenzo[d]oxazol-2-yl)amino)-6-methyl-1,4-dihydropyrimidine-5-carboxamido)isonicotinate as starting material. MS m/z (M+H⁺) 521.1, ¹H NMR (400 MHz, DMSO-d₆) δ 13.59 (s, 1H), 10.65 (s, 1H), 10.18 (d, J=4.1 Hz, 2H), 8.47 (dd, J=5.0, 0.9 Hz, 1H), 8.42 (dd, J=1.5, 0.9 Hz, 1H), 7.59 (dd, J=7.6, 1.8 Hz, 1H), 7.53-7.47 (m, 2H), 7.44-7.29 (m, 4H), 7.02 (ddd, J=10.2, 8.6, 2.6 Hz, 1H), 6.20-6.13 (m, 1H), 2.26 (d, J=0.9 Hz, 3H).

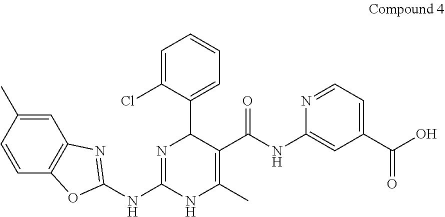 [SMILES: CC1=C(C(=O)Nc2cc(C(=O)O)ccn2)C(c2ccccc2Cl)N=C(Nc2nc3cc(C)ccc3o2)N1]

The title compound 2-(4-(2-chlorophenyl)-6-methyl-2-((5-methylbenzo[d]oxazol-2-yl)amino)-1,4-dihydropyrimidine-5-carboxamido)isonicotinic acid was prepared according to Example 1 using methyl 2-aminoisonicotinate in Step 1 and using 1-(5-methylbenzo[d]oxazol-2-yl)guanidine in Step 2, Example 1a using methyl 2-(4-(2-chlorophenyl)-6-methyl-2-((5-methylbenzo[d]oxazol-2-yl)amino)-1,4-dihydropyrimidine-5-carboxamido)isonicotinate as starting material. MS m/z (M+H⁺) 517.1, ¹H NMR (400 MHz, DMSO-d₆) δ 13.58 (s, 1H), 10.60 (s, 1H), 10.32 (s, 1H), 10.12 (d, J=1.8 Hz, 1H), 8.43 (dd, J=5.0, 0.8 Hz, 1H), 8.39 (t, J=1.1 Hz, 1H), 7.57 (dd, J=7.6, 1.9 Hz, 1H), 7.51-7.43 (m, 2H), 7.39-7.27 (m, 2H), 7.23 (d, J=8.1 Hz, 1H), 7.17-7.14 (m, 1H), 6.90-6.85 (m, 1H), 6.15-6.11 (m, 1H), 2.32 (s, 3H), 2.24 (d, J=0.9 Hz, 3H).

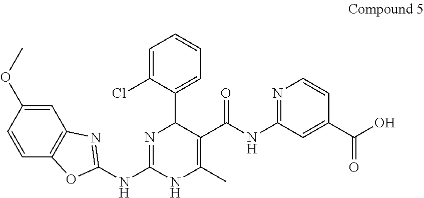 [SMILES: COc1ccc2oc(NC3=NC(c4ccccc4Cl)C(C(=O)Nc4cc(C(=O)O)ccn4)=C(C)N3)nc2c1]

The title compound 2-(4-(2-chlorophenyl)-2-((5-methoxybenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 1-(5-methoxybenzo[d]oxazol-2-yl)guanidine in Step 2, Example 1a using methyl 2-(4-(2-chlorophenyl)-2-((5-methoxybenzo[d]oxazol-2-yl)amino)-6-methyl-1,4-dihydropyrimidine-5-carboxamido)isonicotinate as starting material. MS m/z (M+H⁺) 533.1, ¹H NMR (400 MHz, DMSO-d₆) δ 13.61 (s, 1H), 10.63 (s, 1H), 10.25 (d, J=2.7 Hz, 1H), 10.15 (d, J=1.8 Hz, 1H), 8.46 (dd, J=5.1, 0.8 Hz, 1H), 8.41 (t, J=1.2 Hz, 1H), 7.59 (dd, J=7.6, 1.9 Hz, 1H), 7.54-7.47 (m, 2H), 7.44-7.31 (m, 2H), 7.29 (d, J=8.7 Hz, 1H), 6.94 (d, J=2.5 Hz, 1H), 6.66 (dd, J=8.8, 2.6 Hz, 1H), 6.18-6.14 (m, 1H), 3.76 (s, 3H), 2.26 (d, J=0.9 Hz, 3H).

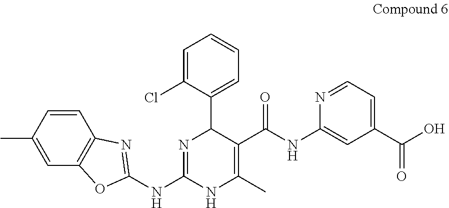 [SMILES: CC1=C(C(=O)Nc2cc(C(=O)O)ccn2)C(c2ccccc2Cl)N=C(Nc2nc3ccc(C)cc3o2)N1]

The title compound 2-(4-(2-chlorophenyl)-6-methyl-2-((6-methylbenzo[d]oxazol-2-yl)amino)-1,4-dihydropyrimidine-5-carboxamido)isonicotinic acid was prepared according to Example 1 using methyl 2-aminoisonicotinate in Step 1 and using 1-(6-methylbenzo[d]oxazol-2-yl)guanidine in Step 2, Example 1a using methyl 2-(4-(2-chlorophenyl)-2-((6-methylbenzo[d]oxazol-2-yl)amino)-6-methyl-1,4-dihydropyrimidine-5-carboxamido)isonicotinate as starting material. MS m/z (M+H⁺) 517.1, ¹H NMR (400 MHz, DMSO-d₆) δ 13.60 (s, 1H), 10.63 (s, 1H), 10.27 (d, J=2.8 Hz, 1H), 10.12 (d, J=1.8 Hz, 1H), 8.47 (dd, J=5.1, 0.8 Hz, 1H), 8.45-8.39 (m, 1H), 7.59 (dd, J=7.6, 1.9 Hz, 1H), 7.53-7.46 (m, 2H), 7.44-7.29 (m, 2H), 7.27-7.20 (m, 2H), 7.03-6.96 (m, 1H), 6.16 (d, J=2.8 Hz, 1H), 2.35 (s, 3H), 2.27 (d, J=0.8 Hz, 3H).

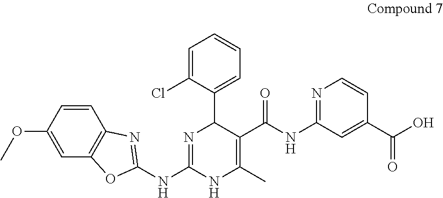 [SMILES: COc1ccc2nc(NC3=NC(c4ccccc4Cl)C(C(=O)Nc4cc(C(=O)O)ccn4)=C(C)N3)oc2c1]

The title compound 2-(4-(2-chlorophenyl)-6-methyl-2-((6-methoxybenzo[d]oxazol-2-yl)amino)-1,4-dihydropyrimidine-5-carboxamido)isonicotinic acid was prepared according to Example 1 using methyl 2-aminoisonicotinate in Step 1 and using 1-(6-methoxybenzo[d]oxazol-2-yl)guanidine in Step 2, Example 1a using methyl 2-(4-(2-chlorophenyl)-2-((6-methoxybenzo[d]oxazol-2-yl)amino)-6-methyl-1,4-dihydropyrimidine-5-carboxamido)isonicotinate as starting material. MS m/z (M+H⁺) 533.2, ¹H NMR (400 MHz, DMSO-d₆) δ 13.60 (s, 1H), 10.62 (s, 1H), 10.20 (s, 1H), 10.10 (d, J=1.8 Hz, 1H), 8.46 (dd, J=5.1, 0.9 Hz, 1H), 8.42 (dd, J=1.5, 0.8 Hz, 1H), 7.59 (dd, J=7.6, 1.9 Hz, 1H), 7.52-7.46 (m, 2H), 7.42-7.29 (m, 2H), 7.26 (d, J=8.6 Hz, 1H), 7.08 (d, J=2.4 Hz, 1H), 6.77 (dd, J=8.6, 2.4 Hz, 1H), 6.18-6.12 (m, 1H), 3.75 (s, 3H), 2.27 (d, J=0.9 Hz, 3H).

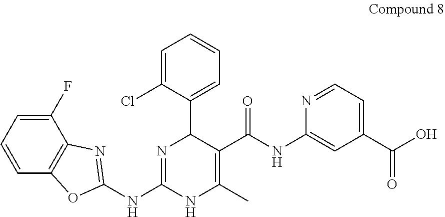 [SMILES: CC1=C(C(=O)Nc2cc(C(=O)O)ccn2)C(c2ccccc2Cl)N=C(Nc2nc3c(F)cccc3o2)N1]

The title compound 2-(4-(2-chlorophenyl)-6-methyl-2-((4-fluorobenzo[d]oxazol-2-yl)amino)-1,4-dihydropyrimidine-5-carboxamido)isonicotinic acid was prepared according to Example 1 using methyl 2-aminoisonicotinate in Step 1 and using 1-(4-fluorobenzo[d]oxazol-2-yl)guanidine in Step 2, Example 1a using methyl 2-(4-(2-chlorophenyl)-2-((4-fluorobenzo[d]oxazol-2-yl)amino)-6-methyl-1,4-dihydropyrimidine-5-carboxamido)isonicotinate as starting material. MS m/z (M+H⁺) 521.1

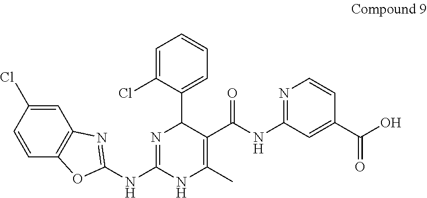 [SMILES: CC1=C(C(=O)Nc2cc(C(=O)O)ccn2)C(c2ccccc2Cl)N=C(Nc2nc3cc(Cl)ccc3o2)N1]

The title compound 2-(2-((5-chlorobenzo[d]oxazol-2-yl)amino)-4-(2-chlorophenyl)-6-methyl-1,4-dihydropyrimidine-5-carboxamido)isonicotinic acid was prepared according to Example 1 using methyl 2-aminoisonicotinate in Step 1 and using 1-(5-chlorobenzo[d]oxazol-2-yl)guanidine in Step 2, Example 1a using methyl 2-(2-((5-chlorobenzo[d]oxazol-2-yl)amino)-4-(2-chlorophenyl)-6-methyl-1,4-dihydropyrimidine-5-carboxamido)isonicotinate as starting material. MS m/z (M+H⁺) 537.1, ¹H NMR (400 MHz, DMSO-d₆) δ 13.60 (s, 1H), 10.67 (s, 1H), 10.23 (d, J=1.8 Hz, 1H), 10.21 (t, J=2.4 Hz, 1H), 8.46 (dd, J=5.1, 0.8 Hz, 1H), 8.42 (t, J=1.2 Hz, 1H), 7.59 (dd, J=7.5, 1.9 Hz, 1H), 7.53-7.47 (m, 2H), 7.43 (d, J=2.3 Hz, 1H), 7.42 (d, J=4.1 Hz, 1H), 7.41-7.30 (m, 2H), 7.12 (dd, J=8.5, 2.1 Hz, 1H), 6.20-6.15 (m, 1H), 2.26 (d, J=0.9 Hz, 3H).

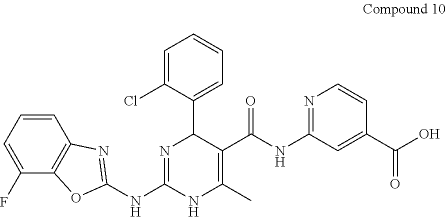 [SMILES: CC1=C(C(=O)Nc2cc(C(=O)O)ccn2)C(c2ccccc2Cl)N=C(Nc2nc3cccc(F)c3o2)N1]

The title compound 2-(4-(2-chlorophenyl)-2-((7-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 1-(7-fluorobenzo[d]oxazol-2-yl)guanidine in Step 2, Example 1a using methyl 2-(4-(2-chlorophenyl)-2-((7-fluorobenzo[d]oxazol-2-yl)amino)-6-methyl-1,4-dihydropyrimidine-5-carboxamido)isonicotinate as starting material. MS m/z (M+H⁺) 521.1, ¹H NMR (400 MHz, DMSO-d₆) δ 13.62 (s, 1H), 10.68 (s, 1H), 10.24 (d, J=1.8 Hz, 1H), 10.16 (t, J=2.4 Hz, 1H), 8.46 (dd, J=5.1, 0.9 Hz, 1H), 8.42 (t, J=1.1 Hz, 1H), 7.60 (dd, J=7.6, 1.9 Hz, 1H), 7.53-7.46 (m, 2H), 7.43-7.30 (m, 2H), 7.25-7.12 (m, 2H), 7.02 (ddd, J=10.5, 8.0, 1.3 Hz, 1H), 6.26-6.08 (m, 1H), 2.27 (d, J=0.9 Hz, 3H).

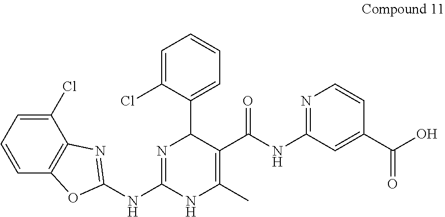 [SMILES: CC1=C(C(=O)Nc2cc(C(=O)O)ccn2)C(c2ccccc2Cl)N=C(Nc2nc3c(Cl)cccc3o2)N1]

The title compound 2-(2-((4-chlorobenzo[d]oxazol-2-yl)amino)-4-(2-chlorophenyl)-6-methyl-1,4-dihydropyrimidine-5-carboxamido)isonicotinic acid was prepared according to Example 1 using methyl 2-aminoisonicotinate in Step 1 and using 1-(4-chlorobenzo[d]oxazol-2-yl)guanidine in Step 2, Example 1a using methyl 2-(2-((4-chlorobenzo[d]oxazol-2-yl)amino)-4-(2-chlorophenyl)-6-methyl-1,4-dihydropyrimidine-5-carboxamido)isonicotinate as starting material. MS m/z (M+H⁺) 537.1, ¹H NMR (400 MHz, DMSO-d₆) δ 13.61 (s, 1H), 10.70 (s, 1H), 10.51 (t, J=2.4 Hz, 1H), 10.26 (d, J=1.8 Hz, 1H), 8.46 (dd, J=5.1, 0.8 Hz, 1H), 8.43 (t, J=1.1 Hz, 1H), 7.62 (dd, J=7.5, 1.9 Hz, 1H), 7.53 (dd, J=7.7, 1.5 Hz, 1H), 7.49 (dd, J=5.1, 1.5 Hz, 1H), 7.43-7.32 (m, 3H), 7.26 (dd, J=8.2, 0.9 Hz, 1H), 7.11 (t, J=8.1 Hz, 1H), 6.22 (d, J=3.0 Hz, 1H), 2.30 (d, J=0.8 Hz, 3H).

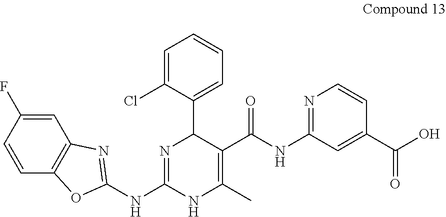 [SMILES: CC1=C(C(=O)Nc2cc(C(=O)O)ccn2)C(c2ccccc2Cl)N=C(Nc2nc3cc(F)ccc3o2)N1]

The title compound 2-(4-(2-chlorophenyl)-2-((5-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 1-(5-fluorobenzo[d]oxazol-2-yl)guanidine in Step 2, Example 1a using methyl 2-(4-(2-chlorophenyl)-2-((5-fluorobenzo[d]oxazol-2-yl)amino)-6-methyl-1,4-dihydropyrimidine-5-carboxamido)isonicotinate as starting material. MS m/z (M+H⁺) 521.1, ¹H NMR (400 MHz, DMSO-d₆) δ 13.53 (s, 1H), 10.62 (s, 1H), 10.18 (s, 2H), 8.43 (d, J=5.1 Hz, 1H), 8.38 (s, 1H), 7.56 (dd, J=7.6, 1.9 Hz, 1H), 7.50-7.44 (m, 2H), 7.42-7.28 (m, 2H), 7.18 (dd, J=8.9, 2.6 Hz, 1H), 7.04 (d, J=9.9 Hz, 1H), 6.95-6.82 (m, 1H), 6.14 (s, 1H), 2.23 (s, 3H).

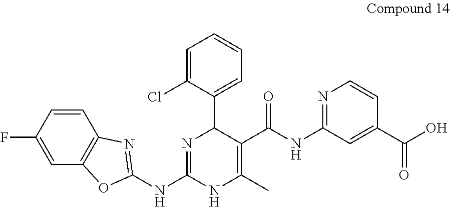 [SMILES: CC1=C(C(=O)Nc2cc(C(=O)O)ccn2)C(c2ccccc2Cl)N=C(Nc2nc3ccc(F)cc3o2)N1]

The title compound 2-(2-((6-chlorobenzo[d]oxazol-2-yl)amino)-4-(2-chlorophenyl)-6-methyl-1,4-dihydropyrimidine-5-carboxamido)isonicotinic acid was prepared according to Example 1 using methyl 2-aminoisonicotinate in Step 1 and using 1-(6-chlorobenzo[d]oxazol-2-yl)guanidine in Step 2, Example 1a using methyl 2-(2-((6-chlorobenzo[d]oxazol-2-yl)amino)-4-(2-chlorophenyl)-6-methyl-1,4-dihydropyrimidine-5-carboxamido)isonicotinate as starting material. MS m/z (M+H⁺) 537.1, ¹H NMR (400 MHz, DMSO-d₆) δ 13.60 (s, 1H), 10.67 (s, 1H), 10.20 (dt, J=4.9, 2.0 Hz, 2H), 8.47 (dd, J=5.1, 0.8 Hz, 1H), 8.42 (t, J=1.1 Hz, 1H), 7.60 (dd, J=7.6, 1.9 Hz, 1H), 7.57 (d, J=2.0 Hz, 1H), 7.50 (dd, J=2.7, 1.5 Hz, 1H), 7.49 (d, J=1.5 Hz, 1H), 7.41-7.31 (m, 3H), 7.21 (dd, J=8.4, 2.1 Hz, 1H), 6.17 (d, J=2.8 Hz, 1H), 2.26 (s, 3H).

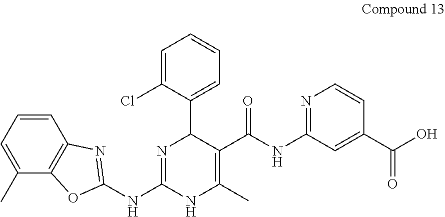 [SMILES: CC1=C(C(=O)Nc2cc(C(=O)O)ccn2)C(c2ccccc2Cl)N=C(Nc2nc3cccc(C)c3o2)N1]

The title compound 2-(4-(2-chlorophenyl)-6-methyl-2-((7-methylbenzo[d]oxazol-2-yl)amino)-1,4-dihydropyrimidine-5-carboxamido)isonicotinic acid was prepared according to Example 1 using methyl 2-aminoisonicotinate in Step 1 and using 1-(7-methylbenzo[d]oxazol-2-yl)guanidine in Step 2, Example 1a using methyl 2-(4-(2-chlorophenyl)-6-methyl-2-((7-methylbenzo[d]oxazol-2-yl)amino)-1,4-dihydropyrimidine-5-carboxamido)isonicotinate as starting material. MS m/z (M+H⁺) 517.1, ¹H NMR (400 MHz, DMSO-d₆) δ 13.60 (s, 1H), 10.64 (s, 1H), 10.27 (s, 1H), 10.10 (d, J=1.8 Hz, 1H), 8.46 (dd, J=5.1, 0.9 Hz, 1H), 8.42 (dd, J=1.5, 0.9 Hz, 1H), 7.59 (dd, J=7.6, 1.8 Hz, 1H), 7.50 (dd, J=3.1, 1.4 Hz, 1H), 7.48 (d, J=1.5 Hz, 1H), 7.38 (td, J=7.5, 1.5 Hz, 1H), 7.33 (td, J=7.6, 1.9 Hz, 1H), 7.18 (ddd, J=7.8, 1.2, 0.7 Hz, 1H), 7.06 (t, J=7.7 Hz, 1H), 6.92 (dt, J=7.5, 1.1 Hz, 1H), 6.29-6.06 (m, 1H), 2.36 (s, 3H), 2.28 (d, J=0.9 Hz, 3H).

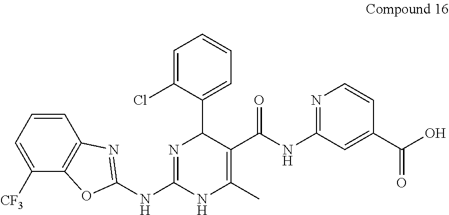 [SMILES: CC1=C(C(=O)Nc2cc(C(=O)O)ccn2)C(c2ccccc2Cl)N=C(Nc2nc3cccc(C(F)(F)F)c3o2)N1]

The title compound 2-(4-(2-chlorophenyl)-6-methyl-2-((7-(trifluoromethyl)benzo[d]oxazol-2-yl)amino)-1,4-dihydropyrimidine-5-carboxamido)isonicotinic acid was prepared according to Example 1 using methyl 2-aminoisonicotinate in Step 1 and using 1-(7-(trifluoromethyl)benzo[d]oxazol-2-yl)guanidine in Step 2, Example 1a using methyl 2-(4-(2-chlorophenyl)-6-methyl-2-((7-(trifluoromethyl)benzo[d]oxazol-2-yl)amino)-1,4-dihydropyrimidine-5-carboxamido)isonicotinate as starting material. MS m/z (M+H⁺) 571.1, ¹H NMR (400 MHz, DMSO-d₆) δ 13.62 (s, 1H), 10.71 (s, 1H), 10.24 (d, J=1.8 Hz, 1H), 10.08 (t, J=2.4 Hz, 1H), 8.47 (dd, J=5.0, 0.8 Hz, 1H), 8.42 (dd, J=1.5, 0.9 Hz, 1H), 7.66 (ddt, J=7.7, 1.4, 0.7 Hz, 1H), 7.60 (dd, J=7.6, 1.9 Hz, 1H), 7.51 (dd, J=2.4, 1.4 Hz, 1H), 7.49 (d, J=1.5 Hz, 1H), 7.43-7.32 (m, 4H), 6.19 (dd, J=3.0, 1.1 Hz, 1H), 2.27 (d, J=0.9 Hz, 3H).

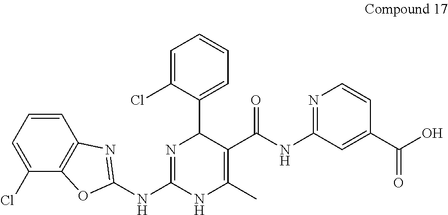 [SMILES: CC1=C(C(=O)Nc2cc(C(=O)O)ccn2)C(c2ccccc2Cl)N=C(Nc2nc3cccc(Cl)c3o2)N1]

The title compound 2-(2-((7-chlorobenzo[d]oxazol-2-yl)amino)-4-(2-chlorophenyl)-6-methyl-1,4-dihydropyrimidine-5-carboxamido)isonicotinic acid was prepared according to Example 1 using methyl 2-aminoisonicotinate in Step 1 and using 1-(7-chlorobenzo[d]oxazol-2-yl)guanidine in Step 2, Example 1a using methyl 2-(2-((7-chlorobenzo[d]oxazol-2-yl)amino)-4-(2-chlorophenyl)-6-methyl-1,4-dihydropyrimidine-5-carboxamido)isonicotinate as starting material. MS m/z (M+H⁺) 537.1, ¹H NMR (400 MHz, DMSO-d₆) δ 13.61 (s, 1H), 10.69 (s, 1H), 10.22 (d, J=1.8 Hz, 1H), 10.13 (t, J=2.3 Hz, 1H), 8.47 (dd, J=5.1, 0.9 Hz, 1H), 8.42 (t, J=1.1 Hz, 1H), 7.60 (dd, J=7.6, 1.9 Hz, 1H), 7.51 (dd, J=2.7, 1.5 Hz, 1H), 7.49 (d, J=1.5 Hz, 1H), 7.42-7.31 (m, 3H), 7.21-7.16 (m, 2H), 6.21-6.16 (m, 1H), 2.27 (d, J=0.9 Hz, 3H).

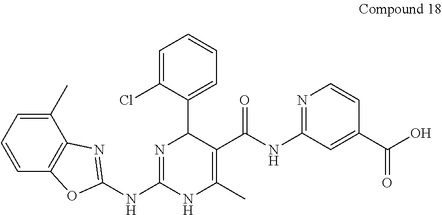 [SMILES: CC1=C(C(=O)Nc2cc(C(=O)O)ccn2)C(c2ccccc2Cl)N=C(Nc2nc3c(C)cccc3o2)N1]

The title compound 2-(4-(2-chlorophenyl)-6-methyl-2-((4-methylbenzo[d]oxazol-2-yl)amino)-1,4-dihydropyrimidine-5-carboxamido)isonicotinic acid was prepared according to Example 1 using methyl 2-aminoisonicotinate in Step 1 and using 1-(4-methylbenzo[d]oxazol-2-yl)guanidine in Step 2, Example 1a using methyl 2-(4-(2-chlorophenyl)-6-methyl-2-((4-methylbenzo[d]oxazol-2-yl)amino)-1,4-dihydropyrimidine-5-carboxamido)isonicotinate as starting material. MS m/z (M+H⁺) 517.2, ¹H NMR (400 MHz, DMSO-d₆) δ 13.61 (s, 1H), 10.65 (s, 1H), 10.63 (s, 1H), 10.14 (d, J=1.8 Hz, 1H), 8.46 (dd, J=5.0, 0.9 Hz, 1H), 8.44-8.42 (m, 1H), 7.63 (dd, J=7.6, 1.9 Hz, 1H), 7.53 (dd, J=7.7, 1.5 Hz, 1H), 7.49 (dd, J=5.1, 1.5 Hz, 1H), 7.40 (td, J=7.5, 1.5 Hz, 1H), 7.35 (td, J=7.5, 1.9 Hz, 1H), 7.24-7.18 (m, 1H), 7.04-6.97 (m, 2H), 6.19 (dd, J=3.0, 1.0 Hz, 1H), 2.45 (d, J=0.8 Hz, 3H), 2.31 (d, J=0.8 Hz, 3H).

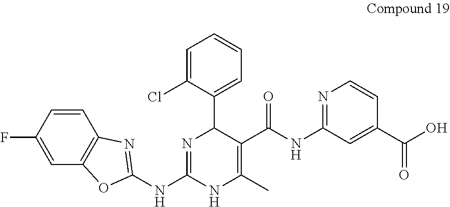 [SMILES: CC1=C(C(=O)Nc2cc(C(=O)O)ccn2)C(c2ccccc2Cl)N=C(Nc2nc3ccc(F)cc3o2)N1]

The title compound (R)-2-(4-(2-chlorophenyl)-2-((6-fluorobenzo[d]oxazol-2-yl)amino)-6-methyl-1,4-dihydropyrimidine-5-carboxamido)isonicotinic acid was prepared according to Example 1a using (R)-methyl 2-(4-(2-chlorophenyl)-2-((6-fluorobenzo[d]oxazol-2-yl)amino)-6-methyl-1,4-dihydropyrimidine-5-carboxamido)isonicotinate as starting material. MS m/z (M+H⁺) 521.1, ¹H NMR (400 MHz, DMSO-d₆) δ 10.66 (s, 1H), 10.18 (d, J=2.5 Hz, 2H), 8.47 (dd, J=5.1, 0.9 Hz, 1H), 8.44-8.41 (m, 1H), 7.59 (dd, J=7.6, 1.9 Hz, 1H), 7.53-7.46 (m, 2H), 7.43-7.30 (m, 4H), 7.03 (ddd, J=10.1, 8.6, 2.5 Hz, 1H), 6.16 (dd, J=2.8, 1.1 Hz, 1H), 2.27 (d, J=0.9 Hz, 3H).

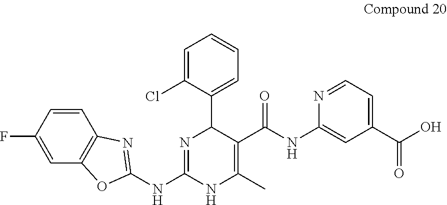 [SMILES: CC1=C(C(=O)Nc2cc(C(=O)O)ccn2)C(c2ccccc2Cl)N=C(Nc2nc3ccc(F)cc3o2)N1]

The title compound (S)-2-(4-(2-chlorophenyl)-2-((6-fluorobenzo[d]oxazol-2-yl)amino)-6-methyl-1,4-dihydropyrimidine-5-carboxamido)isonicotinic acid was prepared according to Example 1a using (S)-methyl 2-(4-(2-chlorophenyl)-2-((6-fluorobenzo[d]oxazol-2-yl)amino)-6-methyl-1,4-dihydropyrimidine-5-carboxamido)isonicotinate as starting material. MS m/z (M+H⁺) 521.1, ¹H NMR (400 MHz, DMSO-d₆) δ 10.66 (s, 1H), 10.18 (d, J=2.6 Hz, 2H), 8.47 (dd, J=5.1, 0.9 Hz, 1H), 8.42 (dd, J=1.5, 0.9 Hz, 1H), 7.59 (dd, J=7.6, 1.9 Hz, 1H), 7.50 (dt, J=6.4, 1.4 Hz, 2H), 7.43-7.30 (m, 4H), 7.03 (ddd, J=10.2, 8.6, 2.5 Hz, 1H), 6.17 (dd, J=2.7, 1.1 Hz, 1H), 2.27 (d, J=0.9 Hz, 3H).

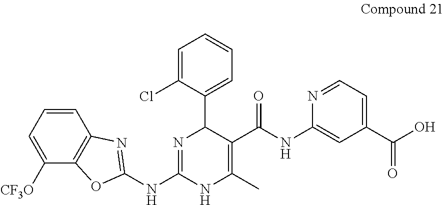

The title compound 2-(4-(2-chlorophenyl)-6-methyl-2-((7-(trifluoromethoxy)benzo[d]oxazol-2-yl)amino)-1,4-dihydropyrimidine-5-carboxamido)isonicotinic acid was prepared according to Example 1 using methyl 2-aminoisonicotinate in Step 1 and using 1-(7-(trifluoromethoxy)benzo[d]oxazol-2-yl)guanidine in Step 2, Example 1a using methyl 2-(4-(2-chlorophenyl)-6-methyl-2-((7-(trifluoromethoxy)benzo[d]oxazol-2-yl)amino)-1,4-dihydropyrimidine-5-carboxamido)isonicotinate as starting material. MS m/z (M+H⁺) 587.1, ¹H NMR (400 MHz, DMSO-d₆) δ 13.61 (s, 1H), 10.71 (s, 1H), 10.24 (d, J=1.8 Hz, 1H), 10.09 (d, J=3.0 Hz, 1H), 8.48 (dd, J=5.0, 0.8 Hz, 1H), 8.43 (t, J=1.1 Hz, 1H), 7.60 (dd, J=7.6, 1.9 Hz, 1H), 7.50 (dt, J=6.2, 1.4 Hz, 2H), 7.39 (ddd, J=9.1, 7.7, 1.3 Hz, 2H), 7.34 (td, J=7.5, 1.9 Hz, 1H), 7.29-7.21 (m, 1H), 7.21-7.12 (m, 1H), 6.29-6.05 (m, 1H), 2.27 (s, 3H).

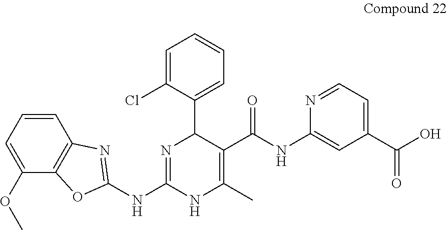 [SMILES: COc1cccc2nc(NC3=NC(c4ccccc4Cl)C(C(=O)Nc4cc(C(=O)O)ccn4)=C(C)N3)oc12]

The title compound 2-(4-(2-chlorophenyl)-2-((7-methoxybenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 1-(7-methoxybenzo[d]oxazol-2-yl)guanidine in Step 2, Example 1a using methyl 2-(4-(2-chlorophenyl)-2-((7-methoxybenzo[d]oxazol-2-yl)amino)-6-methyl-1,4-dihydropyrimidine-5-carboxamido)isonicotinate as starting material. MS m/z (M+H⁺) 533.2, ¹H NMR (400 MHz, DMSO-d₆) δ 13.57 (s, 1H), 10.62 (s, 1H), 10.24 (s, 1H), 10.13 (d, J=1.8 Hz, 1H), 8.44 (dd, J=5.1, 0.8 Hz, 1H), 8.39 (t, J=1.1 Hz, 1H), 7.57 (dd, J=7.6, 1.8 Hz, 1H), 7.49-7.44 (m, 2H), 7.33 (dtd, J=20.2, 7.4, 1.6 Hz, 2H), 7.07 (t, J=8.1 Hz, 1H), 6.94 (dd, J=7.9, 0.9 Hz, 1H), 6.75 (dd, J=8.3, 1.0 Hz, 1H), 6.29-6.02 (m, 1H), 3.86 (s, 3H), 2.24 (d, J=0.9 Hz, 3H).

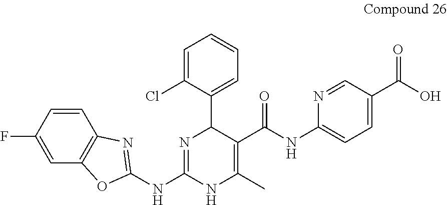 [SMILES: CC1=C(C(=O)Nc2ccc(C(=O)O)cn2)C(c2ccccc2Cl)N=C(Nc2nc3ccc(F)cc3o2)N1]

The title compound 6-(4-(2-chlorophenyl)-2-((6-fluorobenzo[d]oxazol-2-yl)amino)-6-methyl-1,4-dihydropyrimidine-5-carboxamido)nicotinic acid was prepared according to Example 1 using methyl 6-aminonicotinate in Step 1 and using 1-(6-fluorobenzo[d]oxazol-2-yl)guanidine in Step 2, Example 1a using methyl 6-(4-(2-chlorophenyl)-2-((6-fluorobenzo[d]oxazol-2-yl)amino)-6-methyl-1,4-dihydropyrimidine-5-carboxamido)nicotinate as starting material. MS m/z (M+H⁺) 521.1, ¹H NMR (400 MHz, DMSO-d₆) δ 13.12 (s, 1H), 10.79 (s, 1H), 10.19 (d, J=8.4 Hz, 2H), 8.79 (dd, J=2.3, 0.8 Hz, 1H), 8.17 (dd, J=8.7, 2.3 Hz, 1H), 8.02 (dd, J=8.8, 0.9 Hz, 1H), 7.57 (dd, J=7.6, 1.9 Hz, 1H), 7.51-7.44 (m, 1H), 7.43-7.25 (m, 4H), 7.01 (ddd, J=10.1, 8.6, 2.5 Hz, 1H), 6.15 (d, J=2.8 Hz, 1H), 2.25 (d, J=0.8 Hz, 3H).

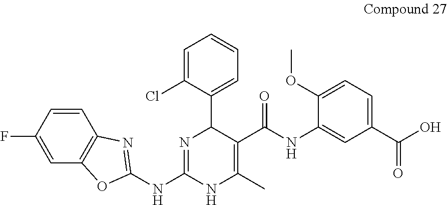 [SMILES: COc1ccc(C(=O)O)cc1NC(=O)C1=C(C)NC(Nc2nc3ccc(F)cc3o2)=NC1c1ccccc1Cl]

The title compound 3-(4-(2-chlorophenyl)-2-((6-fluorobenzo[d]oxazol-2-yl)amino)-6-methyl-1,4-dihydropyrimidine-5-carboxamido)-4-methoxybenzoic acid was prepared according to Example 1 using methyl 3-amino-4-methoxybenzoate in Step 1 and using 1-(6-fluorobenzo[d]oxazol-2-yl)guanidine in Step 2, Example 1a using methyl 3-(4-(2-chlorophenyl)-2-((6-fluorobenzo[d]oxazol-2-yl)amino)-6-methyl-1,4-dihydropyrimidine-5-carboxamido)-4-methoxybenzoate as starting material. MS m/z (M+H⁺) 550.1, ¹H NMR (400 MHz, DMSO-d₆) δ 12.63 (s, 1H), 10.22-10.14 (m, 2H), 8.84 (s, 1H), 8.34 (d, J=2.2 Hz, 1H), 7.67 (dd, J=8.6, 2.2 Hz, 1H), 7.54 (ddd, J=7.4, 4.0, 2.3 Hz, 2H), 7.41-7.29 (m, 4H), 7.10-6.95 (m, 2H), 6.08 (dd, J=2.9, 1.0 Hz, 1H), 3.80 (s, 3H), 2.33 (d, J=0.9 Hz, 3H).

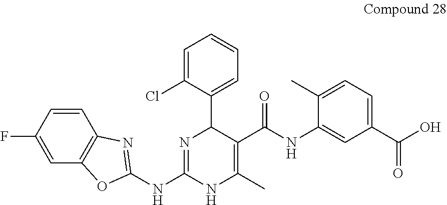 [SMILES: CC1=C(C(=O)Nc2cc(C(=O)O)ccc2C)C(c2ccccc2Cl)N=C(Nc2nc3ccc(F)cc3o2)N1]

The title compound 3-(4-(2-chlorophenyl)-2-((6-fluorobenzo[d]oxazol-2-yl)amino)-6-methyl-1,4-dihydropyrimidine-5-carboxamido)-4-methylbenzoic acid was prepared according to Example 1 using methyl 3-amino-4-methylbenzoate in Step 1 and using 1-(6-fluorobenzo[d]oxazol-2-yl)guanidine in Step 2, Example 1a using methyl 3-(4-(2-chlorophenyl)-2-((6-fluorobenzo[d]oxazol-2-yl)amino)-6-methyl-1,4-dihydropyrimidine-5-carboxamido)-4-methylbenzoate as starting material. MS m/z (M+H⁺) 534.1

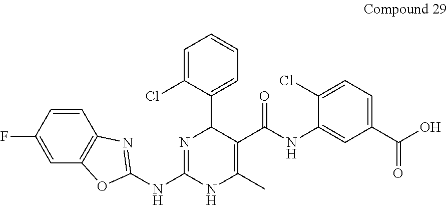 [SMILES: CC1=C(C(=O)Nc2cc(C(=O)O)ccc2Cl)C(c2ccccc2Cl)N=C(Nc2nc3ccc(F)cc3o2)N1]

The title compound 4-chloro-3-(4-(2-chlorophenyl)-2-((6-fluorobenzo[d]oxazol-2-yl)amino)-6-methyl-1,4-dihydropyrimidine-5-carboxamido)benzoic acid was prepared according to Example 1 using methyl 3-amino-4-chlorobenzoate in Step 1 and using 1-(6-fluorobenzo[d]oxazol-2-yl)guanidine in Step 2, Example 1a using methyl 4-chloro-3-(4-(2-chlorophenyl)-2-((6-fluorobenzo[d]oxazol-2-yl)amino)-6-methyl-1,4-dihydropyrimidine-5-carboxamido)benzoate as starting material. MS m/z (M+H⁺) 554.1, ¹H NMR (400 MHz, DMSO-d₆) δ 13.21 (s, 1H), 10.25-10.16 (m, 2H), 9.53 (s, 1H), 8.06 (d, J=2.0 Hz, 1H), 7.69 (dd, J=8.4, 2.1 Hz, 1H), 7.61-7.45 (m, 3H), 7.43-7.29 (m, 4H), 7.01 (ddd, J=10.1, 8.6, 2.5 Hz, 1H), 6.13-6.04 (m, 1H), 2.34 (d, J=0.9 Hz, 3H).

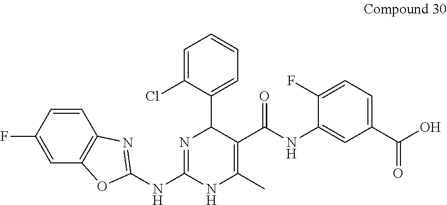 [SMILES: CC1=C(C(=O)Nc2cc(C(=O)O)ccc2F)C(c2ccccc2Cl)N=C(Nc2nc3ccc(F)cc3o2)N1]

The title compound 3-(4-(2-chlorophenyl)-2-((6-fluorobenzo[d]oxazol-2-yl)amino)-6-methyl-1,4-dihydropyrimidine-5-carboxamido)-4-fluorobenzoic acid was prepared according to Example 1 using methyl 3-amino-4-fluorobenzoate in Step 1 and using 1-(6-fluorobenzo[d]oxazol-2-yl)guanidine in Step 2, Example 1a using methyl 3-(4-(2-chlorophenyl)-2-((6-fluorobenzo[d]oxazol-2-yl)amino)-6-methyl-1,4-dihydropyrimidine-5-carboxamido)-4-fluorobenzoate as starting material. MS m/z (M+H⁺) 538.1

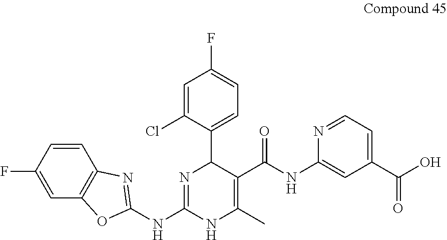 [SMILES: CC1=C(C(=O)Nc2cc(C(=O)O)ccn2)C(c2ccc(F)cc2Cl)N=C(Nc2nc3ccc(F)cc3o2)N1]

The title compound 2-(4-(2-chloro-4-fluorophenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 2-chloro-4-fluorobenzaldehyde and 1-(6-fluorobenzo[d]oxazol-2-yl)guanidine in Step 2, Example 1a using methyl 2-(4-(2-chloro-4-fluorophenyl)-2-((6-fluorobenzo[d]oxazol-2-yl)amino)-6-methyl-1,4-dihydropyrimidine-5-carboxamido)isonicotinate as starting material. MS m/z (M+H⁺) 539.1, ¹H NMR (400 MHz, DMSO-d₆) δ 13.61 (s, 1H), 10.68 (s, 1H), 10.20 (d, J=1.8 Hz, 1H), 10.16 (t, J=2.4 Hz, 1H), 8.47 (dd, J=5.0, 0.9 Hz, 1H), 8.42 (dd, J=1.5, 0.8 Hz, 1H), 7.62 (dd, J=8.8, 6.2 Hz, 1H), 7.54-7.47 (m, 2H), 7.41 (dd, J=8.6, 2.5 Hz, 1H), 7.36 (dd, J=8.6, 5.0 Hz, 1H), 7.27 (td, J=8.5, 2.7 Hz, 1H), 7.03 (ddd, J=10.1, 8.6, 2.5 Hz, 1H), 6.15-6.09 (m, 1H), 2.26 (d, J=0.8 Hz, 3H).

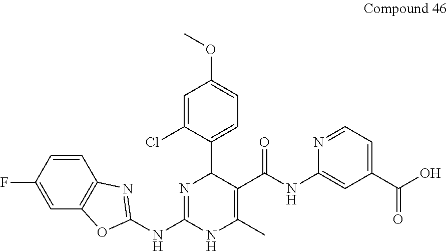 [SMILES: COc1ccc(C2N=C(Nc3nc4ccc(F)cc4o3)NC(C)=C2C(=O)Nc2cc(C(=O)O)ccn2)c(Cl)c1]

The title compound 2-(4-(2-chloro-4-methoxyphenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 2-chloro-4-methoxybenzaldehyde and 1-(6-fluorobenzo[d]oxazol-2-yl)guanidine in Step 2, Example 1a using methyl 2-(4-(2-chloro-4-methoxyphenyl)-2-((6-fluorobenzo[d]oxazol-2-yl)amino)-6-methyl-1,4-dihydropyrimidine-5-carboxamido)isonicotinate as starting material. MS m/z (M+H⁺) 551.1, ¹H NMR (400 MHz, DMSO-d₆) δ 13.60 (s, 1H), 10.62 (s, 1H), 10.14 (d, J=1.8 Hz, 1H), 10.11 (t, J=2.3 Hz, 1H), 8.47 (dd, J=5.0, 0.9 Hz, 1H), 8.43 (dd, J=1.5, 0.8 Hz, 1H), 7.53-7.46 (m, 2H), 7.40 (dd, J=8.6, 2.5 Hz, 1H), 7.35 (dd, J=8.6, 5.0 Hz, 1H), 7.07 (d, J=2.6 Hz, 1H), 7.02 (ddd, J=10.1, 8.6, 2.5 Hz, 1H), 6.93 (dd, J=8.7, 2.6 Hz, 1H), 6.11 (dd, J=2.8, 1.1 Hz, 1H), 3.74 (s, 3H), 2.25 (d, J=0.9 Hz, 3H).

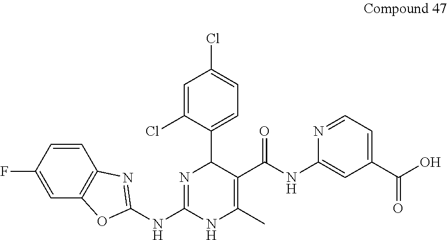 [SMILES: CC1=C(C(=O)Nc2cc(C(=O)O)ccn2)C(c2ccc(Cl)cc2Cl)N=C(Nc2nc3ccc(F)cc3o2)N1]

The title compound 2-(4-(2,4-dichlorophenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 2,4-dichlorobenzaldehyde and 1-(6-fluorobenzo[d]oxazol-2-yl)guanidine in Step 2, Example 1a using methyl 2-(4-(2,4-dichlorophenyl)-2-((6-fluorobenzo[d]oxazol-2-yl)amino)-6-methyl-1,4-dihydropyrimidine-5-carboxamido)isonicotinate as starting material. MS m/z (M+H⁺) 555.1, ¹H NMR (400 MHz, DMSO-d₆) δ 13.62 (s, 1H), 10.67 (s, 1H), 10.22 (d, J=1.8 Hz, 1H), 10.16 (d, J=2.9 Hz, 1H), 8.47 (dd, J=5.1, 0.8 Hz, 1H), 8.42 (t, J=1.1 Hz, 1H), 7.68 (d, J=2.1 Hz, 1H), 7.58 (d, J=8.4 Hz, 1H), 7.50 (dd, J=5.1, 1.5 Hz, 1H), 7.47 (dd, J=8.4, 2.1 Hz, 1H), 7.41 (dd, J=8.6, 2.5 Hz, 1H), 7.36 (dd, J=8.6, 5.0 Hz, 1H), 7.03 (ddd, J=10.2, 8.6, 2.6 Hz, 1H), 6.15-6.07 (m, 1H), 2.25 (d, J=0.9 Hz, 3H).

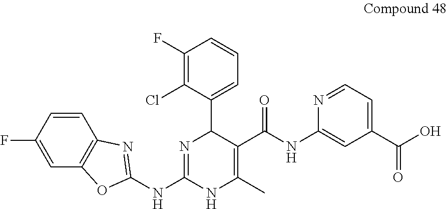 [SMILES: CC1=C(C(=O)Nc2cc(C(=O)O)ccn2)C(c2cccc(F)c2Cl)N=C(Nc2nc3ccc(F)cc3o2)N1]

The title compound 2-(4-(2-chloro-3-fluorophenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 2-chloro-3-fluorobenzaldehyde and 1-(6-fluorobenzo[d]oxazol-2-yl)guanidine in Step 2, Example 1a using methyl 2-(4-(2-chloro-3-fluorophenyl)-2-((6-fluorobenzo[d]oxazol-2-yl)amino)-6-methyl-1,4-dihydropyrimidine-5-carboxamido)isonicotinate as starting material. MS m/z (M+H⁺) 539.1

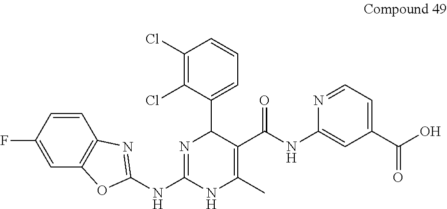 [SMILES: CC1=C(C(=O)Nc2cc(C(=O)O)ccn2)C(c2cccc(Cl)c2Cl)N=C(Nc2nc3ccc(F)cc3o2)N1]

The title compound 2-(4-(2,3-dichlorophenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 2,3-dichlorobenzaldehyde and 1-(6-fluorobenzo[d]oxazol-2-yl)guanidine in Step 2, Example 1a using methyl 2-(4-(2,3-dichlorophenyl)-2-((6-fluorobenzo[d]oxazol-2-yl)amino)-6-methyl-1,4-dihydropyrimidine-5-carboxamido)isonicotinate as starting material. MS m/z (M+H⁺) 555.1, ¹H NMR (400 MHz, DMSO-d₆) δ 13.58 (s, 1H), 10.65 (s, 1H), 10.21 (dd, J=5.5, 2.6 Hz, 2H), 8.44 (dd, J=5.1, 0.9 Hz, 1H), 8.39 (t, J=1.1 Hz, 1H), 7.58 (dd, J=7.9, 1.6 Hz, 1H), 7.53 (dd, J=7.9, 1.6 Hz, 1H), 7.47 (dd, J=5.0, 1.5 Hz, 1H), 7.42-7.32 (m, 3H), 7.01 (ddd, J=10.2, 8.7, 2.6 Hz, 1H), 6.32-6.05 (m, 1H), 2.24 (d, J=0.9 Hz, 3H).

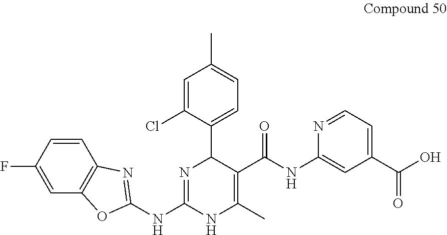 [SMILES: CC1=C(C(=O)Nc2cc(C(=O)O)ccn2)C(c2ccc(C)cc2Cl)N=C(Nc2nc3ccc(F)cc3o2)N1]

The title compound 2-(4-(2-chloro-4-methylphenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 2-chloro-4-methylbenzaldehyde and 1-(6-fluorobenzo[d]oxazol-2-yl)guanidine in Step 2, Example 1a using methyl 2-(4-(2-chloro-4-methylphenyl)-2-((6-fluorobenzo[d]oxazol-2-yl)amino)-6-methyl-1,4-dihydropyrimidine-5-carboxamido)isonicotinate as starting material. MS m/z (M+H⁺) 535.1, ¹H NMR (400 MHz, DMSO-d₆) δ 13.57 (s, 1H), 10.61 (s, 1H), 10.11 (d, J=2.0 Hz, 2H), 8.44 (dd, J=5.0, 0.9 Hz, 1H), 8.39 (dd, J=1.5, 0.9 Hz, 1H), 7.46 (dd, J=5.0, 1.5 Hz, 1H), 7.44 (d, J=7.9 Hz, 1H), 7.37 (dd, J=8.6, 2.5 Hz, 1H), 7.31 (dd, J=8.6, 5.0 Hz, 1H), 7.29 (dd, J=1.7, 0.8 Hz, 1H), 7.17-7.12 (m, 1H), 6.99 (ddd, J=10.2, 8.6, 2.5 Hz, 1H), 6.10 (d, J=2.5 Hz, 1H), 2.22 (d, J=0.9 Hz, 6H).

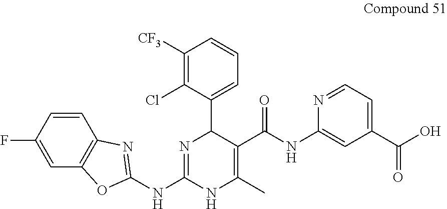 [SMILES: CC1=C(C(=O)Nc2cc(C(=O)O)ccn2)C(c2cccc(C(F)(F)F)c2Cl)N=C(Nc2nc3ccc(F)cc3o2)N1]

The title compound 2-(4-(2-chloro-3-(trifluoromethyl)phenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 2-chloro-3-(trifluoromethyl)benzaldehyde and 1-(6-fluorobenzo[d]oxazol-2-yl)guanidine in Step 2, Example 1a using methyl 2-(4-(2-chloro-3-(trifluoromethyl)phenyl)-2-((6-fluorobenzo[d]oxazol-2-yl)amino)-6-methyl-1,4-dihydropyrimidine-5-carboxamido)isonicotinate as starting material. MS m/z (M+H⁺) 589.1, ¹H NMR (400 MHz, DMSO-d₆) δ 13.58 (s, 1H), 10.68 (s, 1H), 10.25 (d, J=1.7 Hz, 1H), 10.20 (s, 1H), 8.44 (d, J=5.1 Hz, 1H), 8.37 (t, J=1.1 Hz, 1H), 7.86 (d, J=7.9 Hz, 1H), 7.80 (dd, J=7.9, 1.5 Hz, 1H), 7.58 (t, J=7.9 Hz, 1H), 7.47 (dd, J=5.1, 1.4 Hz, 1H), 7.38 (dd, J=8.6, 2.5 Hz, 1H), 7.33 (dd, J=8.6, 5.0 Hz, 1H), 7.01 (ddd, J=10.1, 8.6, 2.6 Hz, 1H), 6.22 (d, J=3.0 Hz, 1H), 2.36-2.12 (m, 3H).

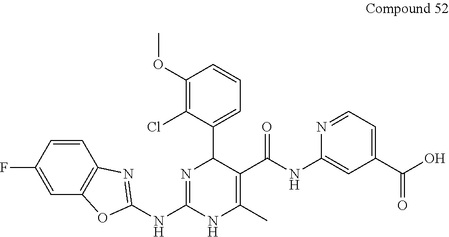 [SMILES: COc1cccc(C2N=C(Nc3nc4ccc(F)cc4o3)NC(C)=C2C(=O)Nc2cc(C(=O)O)ccn2)c1Cl]

The title compound 2-(4-(2-chloro-3-methoxyphenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 2-chloro-3methoxybenzaldehyde and 1-(6-fluorobenzo[d]oxazol-2-yl)guanidine in Step 2, Example 1a using methyl 2-(4-(2-chloro-3-methoxyphenyl)-2-((6-fluorobenzo[d]oxazol-2-yl)amino)-6-methyl-1,4-dihydropyrimidine-5-carboxamido)isonicotinate as starting material. MS m/z (M+H⁺) 551.1, ¹H NMR (400 MHz, DMSO-d₆) δ 13.60 (s, 1H), 10.63 (s, 1H), 10.20 (d, J=2.8 Hz, 1H), 10.15 (s, 1H), 8.50-8.44 (m, 1H), 8.42 (t, J=1.1 Hz, 1H), 7.49 (dd, J=5.1, 1.4 Hz, 1H), 7.40 (dd, J=8.6, 2.5 Hz, 1H), 7.38-7.29 (m, 2H), 7.18 (dd, J=7.9, 1.3 Hz, 1H), 7.10 (dd, J=8.4, 1.3 Hz, 1H), 7.02 (ddd, J=10.1, 8.6, 2.5 Hz, 1H), 6.19 (d, J=2.9 Hz, 1H), 3.83 (s, 3H), 2.26 (s, 3H).

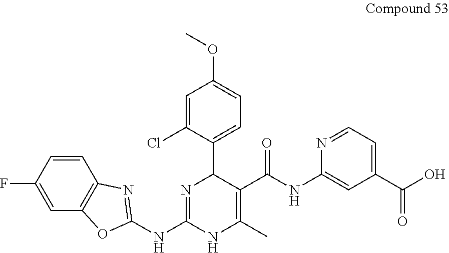 [SMILES: COc1ccc(C2N=C(Nc3nc4ccc(F)cc4o3)NC(C)=C2C(=O)Nc2cc(C(=O)O)ccn2)c(Cl)c1]

The title compound (R)-2-(4-(2-chloro-4-methoxyphenyl)-2-((6-fluorobenzo[d]oxazol-2-yl)amino)-6-methyl-1,4-dihydropyrimidine-5-carboxamido)isonicotinic acid was prepared according to Example 1a using (R)-methyl 2-(4-(2-chloro-4-methoxyphenyl)-2-((6-fluorobenzo[d]oxazol-2-yl)amino)-6-methyl-1,4-dihydropyrimidine-5-carboxamido)isonicotinate as starting material. MS m/z (M+H⁺) 551.2

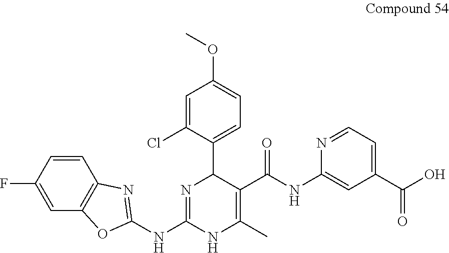 [SMILES: COc1ccc(C2N=C(Nc3nc4ccc(F)cc4o3)NC(C)=C2C(=O)Nc2cc(C(=O)O)ccn2)c(Cl)c1]

The title compound (S)-2-(4-(2-chloro-4-methoxyphenyl)-2-((6-fluorobenzo[d]oxazol-2-yl)amino)-6-methyl-1,4-dihydropyrimidine-5-carboxamido)isonicotinic acid was prepared according to Example 1a using (S)-methyl 2-(4-(2-chlor-4-methoxyphenyl)-2-((6-fluorobenzo[d]oxazol-2-yl)amino)-6-methyl-1,4-dihydropyrimidine-5-carboxamido)isonicotinate as starting material. MS m/z (M+H⁺) 551.2

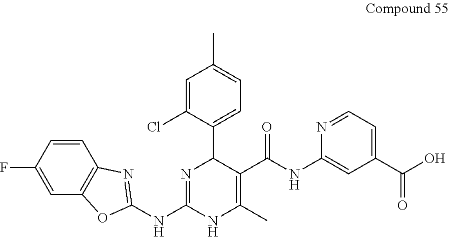 [SMILES: CC1=C(C(=O)Nc2cc(C(=O)O)ccn2)C(c2ccc(C)cc2Cl)N=C(Nc2nc3ccc(F)cc3o2)N1]

The title compound (R)-2-(4-(2-chloro-4-methylphenyl)-2-((6-fluorobenzo[d]oxazol-2-yl)amino)-6-methyl-1,4-dihydropyrimidine-5-carboxamido)isonicotinic acid was prepared according to Example 1a using (R)-methyl 2-(4-(2-chloro-4-methylphenyl)-2-((6-fluorobenzo[d]oxazol-2-yl)amino)-6-methyl-1,4-dihydropyrimidine-5-carboxamido)isonicotinate as starting material. MS m/z (M+H⁺) 535.2, ¹H NMR (400 MHz, DMSO-d₆) δ 13.57 (s, 1H), 10.62 (s, 1H), 10.12 (s, 2H), 8.45 (dd, J=5.1, 0.8 Hz, 1H), 8.40 (dd, J=1.5, 0.8 Hz, 1H), 7.47 (dd, J=5.1, 1.5 Hz, 1H), 7.44 (d, J=7.9 Hz, 1H), 7.37 (dd, J=8.6, 2.5 Hz, 1H), 7.32 (dd, J=8.6, 5.0 Hz, 1H), 7.30-7.28 (m, 1H), 7.15 (dd, J=8.1, 1.7 Hz, 1H), 7.05-6.94 (m, 1H), 6.10 (d, J=2.5 Hz, 1H), 2.23 (s, 6H).

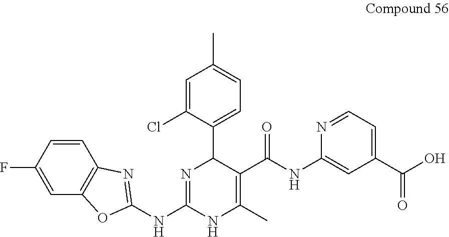 [SMILES: CC1=C(C(=O)Nc2cc(C(=O)O)ccn2)C(c2ccc(C)cc2Cl)N=C(Nc2nc3ccc(F)cc3o2)N1]

The title compound (S)-2-(4-(2-chloro-4-methylphenyl)-2-((6-fluorobenzo[d]oxazol-2-yl)amino)-6-methyl-1,4-dihydropyrimidine-5-carboxamido)isonicotinic acid was prepared according to Example 1a using (S)-methyl 2-(4-(2-chlor-4-methylphenyl)-2-((6-fluorobenzo[d]oxazol-2-yl)amino)-6-methyl-1,4-dihydropyrimidine-5-carboxamido)isonicotinate as starting material. MS m/z (M+H⁺) 551.2, ¹H NMR (400 MHz, DMSO-d₆) δ 13.57 (s, 1H), 10.61 (s, 1H), 10.12 (d, J=2.3 Hz, 2H), 8.44 (d, J=5.1 Hz, 1H), 8.40 (d, J=1.2 Hz, 1H), 7.47 (dd, J=5.0, 1.4 Hz, 1H), 7.44 (d, J=7.9 Hz, 1H), 7.37 (dd, J=8.6, 2.5 Hz, 1H), 7.32 (dd, J=8.6, 5.0 Hz, 1H), 7.29 (dd, J=1.6, 0.7 Hz, 1H), 7.15 (dd, J=8.1, 1.7 Hz, 1H), 7.05-6.95 (m, 1H), 6.10 (d, J=2.4 Hz, 1H), 2.23 (s, 6H).

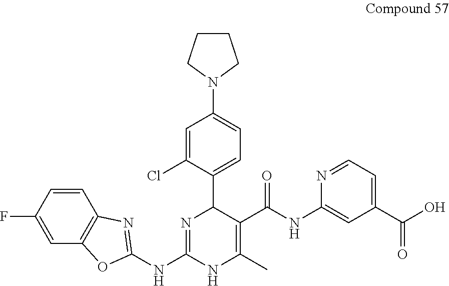 [SMILES: CC1=C(C(=O)Nc2cc(C(=O)O)ccn2)C(c2ccc(N3CCCC3)cc2Cl)N=C(Nc2nc3ccc(F)cc3o2)N1]

The title compound 2-(4-(2-chloro-4-(pyrrolidin-1-yl)phenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 2-chloro-4-(pyrrolidin-1-yl)benzaldehyde and 1-(6-fluorobenzo[d]oxazol-2-yl)guanidine in Step 2, Example 1a using methyl 2-(4-(2-chloro-4-(pyrrolidin-1-yl)phenyl)-2-((6-fluorobenzo[d]oxazol-2-yl)amino)-6-methyl-1,4-dihydropyrimidine-5-carboxamido)isonicotinate as starting material. MS m/z (M+H⁺) 590.2, ¹H NMR (400 MHz, DMSO-d₆) δ 13.58 (s, 1H), 10.49 (s, 1H), 10.03 (s, 1H), 10.00 (s, 1H), 8.44 (d, J=5.1 Hz, 1H), 8.42 (s, 1H), 7.46 (d, J=5.1 Hz, 1H), 7.36 (dd, J=8.6, 2.2 Hz, 1H), 7.30 (t, J=7.6 Hz, 2H), 7.02-6.95 (m, 1H), 6.50 (d, J=1.8 Hz, 1H), 6.47-6.41 (m, 1H), 6.05 (s, 1H), 3.14 (s, 4H), 2.20 (s, 3H), 1.90-1.82 (m, 4H).

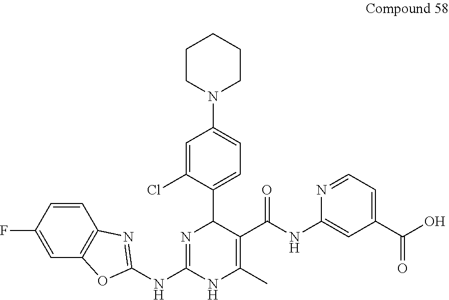 [SMILES: CC1=C(C(=O)Nc2cc(C(=O)O)ccn2)C(c2ccc(N3CCCCC3)cc2Cl)N=C(Nc2nc3ccc(F)cc3o2)N1]

The title compound 2-(4-(2-chloro-4-(piperidin-1-yl)phenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 2-chloro-4-(piperidin-1-yl)benzaldehyde and 1-(6-fluorobenzo[d]oxazol-2-yl)guanidine in Step 2, Example 1a using methyl 2-(4-(2-chloro-4-(piperidin-1-yl)phenyl)-2-((6-fluorobenzo[d]oxazol-2-yl)amino)-6-methyl-1,4-dihydropyrimidine-5-carboxamido)isonicotinate as starting material. MS m/z (M+H⁺) 604.3, ¹H NMR (400 MHz, DMSO-d₆) δ 13.60 (s, 1H), 10.56 (s, 1H), 10.07 (s, 1H), 10.04 (s, 1H), 8.44 (d, J=5.1 Hz, 1H), 8.42 (s, 1H), 7.47 (d, J=5.1 Hz, 1H), 7.37 (dd, J=8.6, 2.1 Hz, 1H), 7.35-7.27 (m, 2H), 7.00 (t, J=9.1 Hz, 1H), 6.92 (s, 1H), 6.85 (d, J=8.4 Hz, 1H), 6.04 (s, 1H), 3.13 (d, J=5.8 Hz, 4H), 2.21 (s, 3H), 1.50 (d, J=8.3 Hz, 6H).

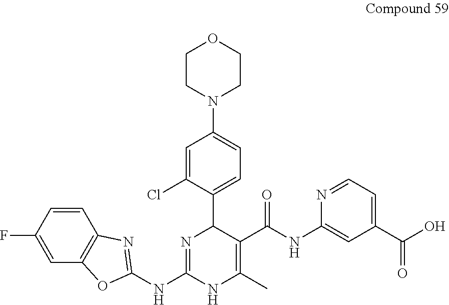 [SMILES: CC1=C(C(=O)Nc2cc(C(=O)O)ccn2)C(c2ccc(N3CCOCC3)cc2Cl)N=C(Nc2nc3ccc(F)cc3o2)N1]

The title compound 2-(4-(2-chloro-4-morpholinphenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 2-chloro-4-morpholinbenzaldehyde and 1-(6-fluorobenzo[d]oxazol-2-yl)guanidine in Step 2, Example 1a using methyl 2-(4-(2-chloro-4-morpholinphenyl)-2-((6-fluorobenzo[d]oxazol-2-yl)amino)-6-methyl-1,4-dihydropyrimidine-5-carboxamido)isonicotinate as starting material. MS m/z (M+H⁺) 606.2, ¹H NMR (400 MHz, DMSO-d₆) δ 13.60 (s, 1H), 10.57 (s, 1H), 10.08 (s, 1H), 10.05 (s, 1H), 8.44 (d, J=5.1 Hz, 1H), 8.41 (s, 1H), 7.47 (d, J=5.1 Hz, 1H), 7.41-7.27 (m, 3H), 6.98 (dd, J=18.8, 10.2 Hz, 2H), 6.87 (d, J=8.5 Hz, 1H), 6.05 (s, 1H), 3.68 (t, J=5.0 Hz, 4H), 3.08 (t, J=5.0 Hz, 4H), 2.21 (s, 3H).

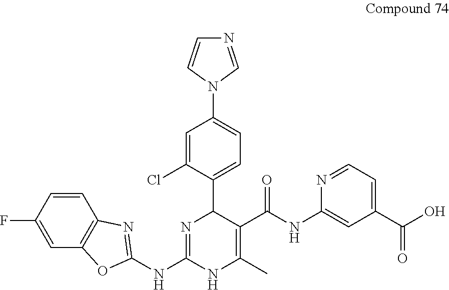 [SMILES: CC1=C(C(=O)Nc2cc(C(=O)O)ccn2)C(c2ccc(-n3ccnc3)cc2Cl)N=C(Nc2nc3ccc(F)cc3o2)N1]

The title compound 2-(4-(2-chloro-4-(1H-imidazol-1-yl)phenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 2-chloro-4-(1H-imidazol-1-yl)benzaldehyde and 1-(6-fluorobenzo[d]oxazol-2-yl)guanidine in Step 2, Example 1a using methyl 2-(4-(2-chloro-4-(1H-imidazol-1-yl)phenyl)-2-((6-fluorobenzo[d]oxazol-2-yl)amino)-6-methyl-1,4-dihydropyrimidine-5-carboxamido)isonicotinate as starting material. MS m/z (M+H⁺) 587.2, ¹H NMR (400 MHz, DMSO-d₆) δ 13.60 (s, 1H), 10.71 (s, 1H), 10.24 (d, J=4.4 Hz, 2H), 9.47 (s, 1H), 8.45 (dd, J=5.1, 0.7 Hz, 1H), 8.41 (dd, J=1.4, 0.9 Hz, 1H), 8.19 (t, J=1.7 Hz, 1H), 8.03-8.01 (m, 1H), 7.77 (t, J=1.7 Hz, 1H), 7.75 (d, J=1.9 Hz, 2H), 7.47 (dd, J=5.2, 1.5 Hz, 1H), 7.38 (dd, J=8.5, 2.5 Hz, 1H), 7.33 (dd, J=8.6, 5.0 Hz, 1H), 7.01 (ddd, J=10.1, 8.5, 2.5 Hz, 1H), 6.17 (d, J=2.8 Hz, 1H), 2.27 (s, 3H).

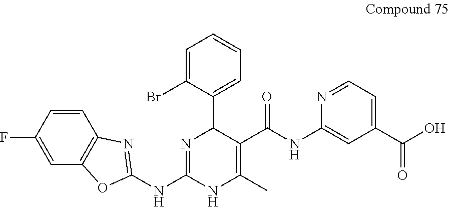 [SMILES: CC1=C(C(=O)Nc2cc(C(=O)O)ccn2)C(c2ccccc2Br)N=C(Nc2nc3ccc(F)cc3o2)N1]

The title compound 2-(4-(2-bromophenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 2-bromobenzaldehyde and 1-(6-fluorobenzo[d]oxazol-2-yl)guanidine in Step 2, Example 1a using methyl 2-(4-(2-bromophenyl)-2-((6-fluorobenzo[d]oxazol-2-yl)amino)-6-methyl-1,4-dihydropyrimidine-5-carboxamido)isonicotinate as starting material. MS m/z (M+H⁺) 567.1, ¹H NMR (400 MHz, DMSO-d₆) δ 10.63 (s, 1H), 10.31 (s, 1H), 10.16 (d, J=1.8 Hz, 1H), 8.44 (d, J=5.1 Hz, 1H), 8.39 (s, 1H), 7.66 (dd, J=8.0, 1.2 Hz, 1H), 7.62 (dd, J=7.8, 1.7 Hz, 1H), 7.48 (dd, J=5.0, 1.5 Hz, 1H), 7.46-7.33 (m, 3H), 7.26 (ddd, J=7.9, 7.3, 1.7 Hz, 1H), 7.03 (ddd, J=10.2, 8.6, 2.5 Hz, 1H), 6.10 (dd, J=3.0, 1.0 Hz, 1H), 2.27 (d, J=0.9 Hz, 3H).

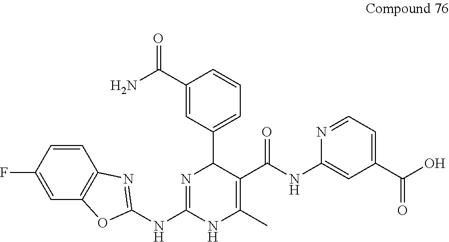 [SMILES: CC1=C(C(=O)Nc2cc(C(=O)O)ccn2)C(c2cccc(C(N)=O)c2)N=C(Nc2nc3ccc(F)cc3o2)N1]

The title compound 2-(4-(3-carbamoylphenyl)-2-((6-fluorobenzo[d]oxazol-2-yl)amino)-6-methyl-1,4-dihydropyrimidine-5-carboxamido)isonicotinic acid was prepared according to Example 1 using methyl 2-aminoisonicotinate in Step 1 and using 3-formylbenzamide and 1-(6-fluorobenzo[d]oxazol-2-yl)guanidine in Step 2, Example 1a using methyl 2-(4-(3-carbamoylphenyl)-2-((6-fluorobenzo[d]oxazol-2-yl)amino)-6-methyl-1,4-dihydropyrimidine-5-carboxamido)isonicotinate as starting material. MS m/z (M+H⁺) 530.1, ¹H NMR (400 MHz, DMSO-d₆) δ 13.62 (s, 1H), 10.47 (s, 1H), 10.10 (s, 1H), 9.70 (d, J=2.8 Hz, 1H), 8.43 (d, J=5.1 Hz, 1H), 8.41 (t, J=1.1 Hz, 1H), 7.97 (s, 1H), 7.90 (t, J=1.8 Hz, 1H), 7.77 (ddd, J=7.7, 1.8, 1.2 Hz, 1H), 7.58-7.53 (m, 1H), 7.48 (dd, J=5.0, 1.5 Hz, 1H), 7.46-7.37 (m, 3H), 7.33 (dd, J=8.7, 5.0 Hz, 1H), 7.00 (ddd, J=10.2, 8.6, 2.6 Hz, 1H), 5.91-5.85 (m, 1H), 2.20 (d, J=0.9 Hz, 3H).

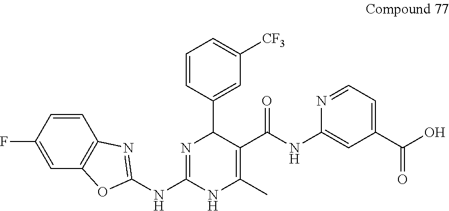 [SMILES: CC1=C(C(=O)Nc2cc(C(=O)O)ccn2)C(c2cccc(C(F)(F)F)c2)N=C(Nc2nc3ccc(F)cc3o2)N1]

The title compound 2-(2-((6-fluorobenzo[d]oxazol-2-yl)amino)-6-methyl-4-(3-(trifluoromethyl)phenyl)-1,4-dihydropyrimidine-5-carboxamido)isonicotinic acid was prepared according to Example 1 using methyl 2-aminoisonicotinate in Step 1 and using 3-(trifluoromethyl)benzaldehyde and 1-(6-fluorobenzo[d]oxazol-2-yl)guanidine in Step 2, Example 1a using methyl 2-(2-((6-fluorobenzo[d]oxazol-2-yl)amino)-6-methyl-4-(3-(trifluoromethyl)phenyl)-1,4-dihydropyrimidine-5-carboxamido)isonicotinate as starting material. MS m/z (M+H⁺) 555.2, ¹H NMR (400 MHz, DMSO-d₆) δ 13.62 (s, 1H), 10.57 (s, 1H), 10.15 (d, J=1.9 Hz, 1H), 9.76 (t, J=2.5 Hz, 1H), 8.45 (dd, J=5.0, 0.9 Hz, 1H), 8.43-8.39 (m, 1H), 7.73 (dt, J=5.2, 1.9 Hz, 2H), 7.69-7.58 (m, 2H), 7.49 (dd, J=5.1, 1.5 Hz, 1H), 7.41 (dd, J=8.6, 2.6 Hz, 1H), 7.34 (dd, J=8.6, 5.0 Hz, 1H), 7.01 (ddd, J=10.2, 8.7, 2.6 Hz, 1H), 5.93 (d, J=3.1 Hz, 1H), 2.20 (d, J=0.9 Hz, 3H).

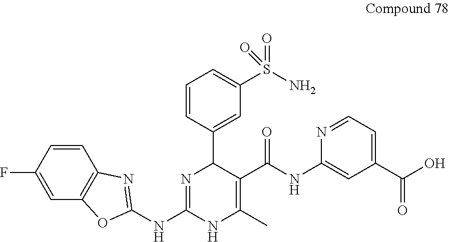 [SMILES: CC1=C(C(=O)Nc2cc(C(=O)O)ccn2)C(c2cccc(S(N)(=O)=O)c2)N=C(Nc2nc3ccc(F)cc3o2)N1]

The title compound 2-(2-((6-fluorobenzo[d]oxazol-2-yl)amino)-6-methyl-4-(3-sulfamoylphenyl)-1,4-dihydropyrimidine-5-carboxamido)isonicotinic acid was prepared according to Example 1 using methyl 2-aminoisonicotinate in Step 1 and using 3-formylbenzenesulfonamide and 1-(6-fluorobenzo[d]oxazol-2-yl)guanidine in Step 2, Example 1a using methyl 2-(2-((6-fluorobenzo[d]oxazol-2-yl)amino)-6-methyl-4-(3-sulfamoylphenyl)-1,4-dihydropyrimidine-5-carboxamido)isonicotinate as starting material. MS m/z (M+H⁺) 566.1, ¹H NMR (400 MHz, DMSO-d₆) δ 13.58 (s, 1H), 10.49 (s, 1H), 10.13 (d, J=1.9 Hz, 1H), 9.81-9.62 (m, 1H), 8.43 (dd, J=5.1, 0.9 Hz, 1H), 8.41 (t, J=1.2 Hz, 1H), 7.85 (t, J=1.8 Hz, 1H), 7.72 (dt, J=7.6, 1.5 Hz, 1H), 7.60 (dt, J=7.8, 1.5 Hz, 1H), 7.54 (t, J=7.7 Hz, 1H), 7.47 (dd, J=5.1, 1.5 Hz, 1H), 7.41-7.34 (m, 3H), 7.32 (dd, J=8.6, 5.0 Hz, 1H), 6.98 (ddd, J=10.1, 8.7, 2.6 Hz, 1H), 5.88 (d, J=3.2 Hz, 1H), 2.19 (s, 3H).

### Example 1b

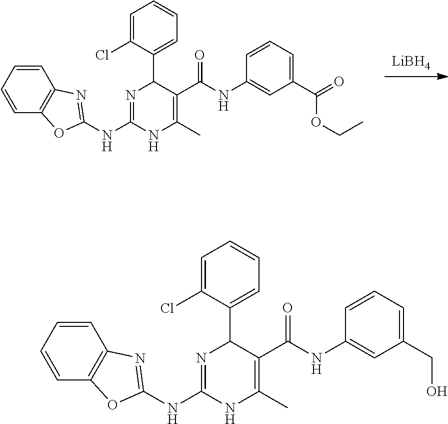 [SMILES: CCOC(=O)c1cccc(NC(=O)C2=C(C)NC(Nc3nc4ccccc4o3)=NC2c2ccccc2Cl)c1 ; "LiBH4" ; CC1=C(C(=O)Nc2cccc(CO)c2)C(c2ccccc2Cl)N=C(Nc2nc3ccccc3o2)N1]

To a solution of ethyl 3-(2-(benzo[d]oxazol-2-ylamino)-4-(2-chlorophenyl)-6-methyl-1,4-dihydropyrimidine-5-carboxamido)benzoate (50 mg, 0.094 mmol) in THF (1 mL) was added dropwise LiBH₄ (0.071 mL, 0.142 mmol) (2.0 M in THF). The mixture was stirred at R.T. overnight.

The solvent was removed. The crude product was purified. (Compound 144). MS m/z (M+H⁺) 488.1.1, ¹H NMR (400 MHz, DMSO-d₆) δ 10.29 (s, 1H), 10.10 (d, J=1.7 Hz, 1H), 9.88 (s, 1H), 7.54 (dt, J=6.0, 1.9 Hz, 2H), 7.49 (dd, J=7.7, 1.4 Hz, 1H), 7.45-7.31 (m, 5H), 7.24-7.14 (m, 2H), 7.10 (td, J=7.7, 1.3 Hz, 1H), 6.96 (ddd, J=7.6, 1.8, 1.0 Hz, 1H), 6.09 (dd, J=3.1, 1.1 Hz, 1H), 4.43 (s, 2H), 2.22 (d, J=0.8 Hz, 3H).

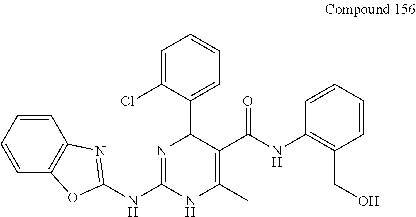 [SMILES: CC1=C(C(=O)Nc2ccccc2CO)C(c2ccccc2Cl)N=C(Nc2nc3ccccc3o2)N1]

The title compound 2-(benzo[d]oxazol-2-ylamino)-4-(2-chlorophenyl)-N-(2-(hydroxymethyl)phenyl)-6-methyl-1,4-dihydropyrimidine-5-carboxamide was prepared according to Example 1b using methyl 2-(2-(benzo[d]oxazol-2-ylamino)-4-(2-chlorophenyl)-6-methyl-1,4-dihydropyrimidine-5-carboxamido)benzoate as starting material. MS m/z (M+H⁺) 488.1, ¹H NMR (400 MHz, DMSO-d₆) δ 10.34-10.28 (m, 1H), 10.15 (d, J=1.8 Hz, 1H), 9.42 (s, 1H), 7.52 (ddd, J=7.3, 5.5, 1.8 Hz, 2H), 7.48-7.43 (m, 1H), 7.43-7.31 (m, 6H), 7.22-7.15 (m, 2H), 7.14-7.07 (m, 2H), 6.05 (d, J=2.8 Hz, 1H), 4.33 (q, J=13.9 Hz, 2H), 2.33 (s, 3H).

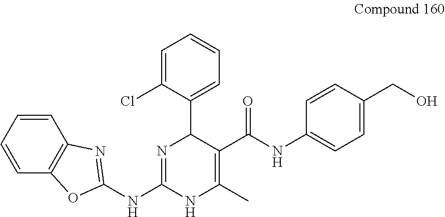 [SMILES: CC1=C(C(=O)Nc2ccc(CO)cc2)C(c2ccccc2Cl)N=C(Nc2nc3ccccc3o2)N1]

The title compound 2-(benzo[d]oxazol-2-ylamino)-4-(2-chlorophenyl)-N-(4-(hydroxymethyl)phenyl)-6-methyl-1,4-dihydropyrimidine-5-carboxamide was prepared according to Example 1b using methyl 4-(2-(benzo[d]oxazol-2-ylamino)-4-(2-chlorophenyl)-6-methyl-1,4-dihydropyrimidine-5-carboxamido)benzoate as starting material. MS m/z (M+H⁺) 488.1

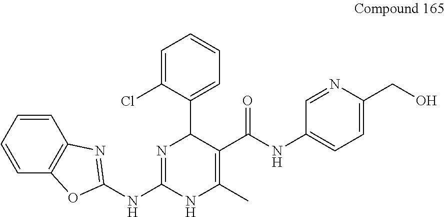 [SMILES: CC1=C(C(=O)Nc2ccc(CO)nc2)C(c2ccccc2Cl)N=C(Nc2nc3ccccc3o2)N1]

The title compound 2-(benzo[d]oxazol-2-ylamino)-4-(2-chlorophenyl)-N-(6-(hydroxymethyl)pyridin-3-yl)-6-methyl-1,4-dihydropyrimidine-5-carboxamide was prepared according to Example 1b using methyl 5-(2-(benzo[d]oxazol-2-ylamino)-4-(2-chlorophenyl)-6-methyl-1,4-dihydropyrimidine-5-carboxamido)picolinate as starting material. MS m/z (M+H⁺) 488.1, ¹H NMR (400 MHz, DMSO-d₆) δ 10.32 (d, J=2.8 Hz, 1H), 10.27 (s, 1H), 10.23 (s, 1H), 8.75 (d, J=2.4 Hz, 1H), 8.10 (dd, J=8.7, 2.4 Hz, 1H), 7.55 (d, J=8.6 Hz, 1H), 7.51 (t, J=2.2 Hz, 1H), 7.49 (dd, J=2.6, 1.7 Hz, 1H), 7.43-7.30 (m, 4H), 7.21-7.15 (m, 1H), 7.11 (td, J=7.6, 1.3 Hz, 1H), 6.09 (d, J=2.9 Hz, 1H), 4.59 (s, 2H), 2.26 (s, 3H).

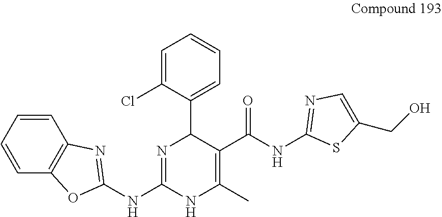 [SMILES: CC1=C(C(=O)Nc2ncc(CO)s2)C(c2ccccc2Cl)N=C(Nc2nc3ccccc3o2)N1]

The title compound 2-(benzo[d]oxazol-2-ylamino)-4-(2-chlorophenyl)-N-(5-(hydroxymethyl)thiazol-2-yl)-6-methyl-1,4-dihydropyrimidine-5-carboxamide was prepared according to Example 1b using methyl 2-(2-(benzo[d]oxazol-2-ylamino)-4-(2-chlorophenyl)-6-methyl-1,4-dihydropyrimidine-5-carboxamido)thiazole-5-carboxylate as starting material. MS m/z (M+H⁺) 495.1

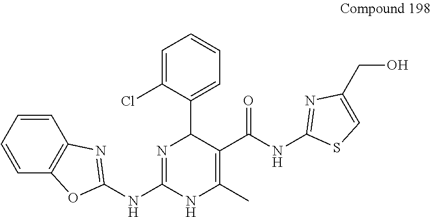 [SMILES: CC1=C(C(=O)Nc2nc(CO)cs2)C(c2ccccc2Cl)N=C(Nc2nc3ccccc3o2)N1]

The title compound 2-(benzo[d]oxazol-2-ylamino)-4-(2-chlorophenyl)-N-(4-(hydroxymethyl)thiazol-2-yl)-6-methyl-1,4-dihydropyrimidine-5-carboxamide was prepared according to Example 1b using methyl 2-(2-(benzo[d]oxazol-2-ylamino)-4-(2-chlorophenyl)-6-methyl-1,4-dihydropyrimidine-5-carboxamido)thiazole-4-carboxylate as starting material. MS m/z (M+H⁺) 495.1, ¹H NMR (400 MHz, DMSO-d₆) δ 12.03 (s, 1H), 10.37 (s, 1H), 10.27 (s, 1H), 7.53-7.44 (m, 2H), 7.37 (ddd, J=15.3, 9.2, 7.4 Hz, 5H), 7.18 (dd, J=8.2, 6.9 Hz, 1H), 7.15-7.07 (m, 1H), 6.88 (s, 1H), 6.19 (d, J=3.0 Hz, 1H), 4.44 (s, 2H), 2.29 (s, 3H).

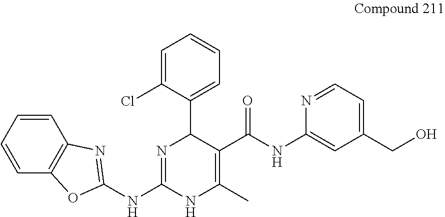 [SMILES: CC1=C(C(=O)Nc2cc(CO)ccn2)C(c2ccccc2Cl)N=C(Nc2nc3ccccc3o2)N1]

The title compound 2-(benzo[d]oxazol-2-ylamino)-4-(2-chlorophenyl)-N-(4-(hydroxymethyl)pyridin-2-yl)-6-methyl-1,4-dihydropyrimidine-5-carboxamide was prepared according to Example 1b using methyl 2-(2-(benzo[d]oxazol-2-ylamino)-4-(2-chlorophenyl)-6-methyl-1,4-dihydropyrimidine-5-carboxamido)isonicotinate as starting material. MS m/z (M+H⁺) 489.1

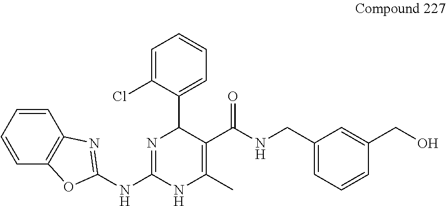 [SMILES: CC1=C(C(=O)NCc2cccc(CO)c2)C(c2ccccc2Cl)N=C(Nc2nc3ccccc3o2)N1]

The title compound 2-(benzo[d]oxazol-2-ylamino)-4-(2-chlorophenyl)-N-(3-(hydroxymethyl)benzyl)-6-methyl-1,4-dihydropyrimidine-5-carboxamide was prepared according to Example 1b using methyl 3-((2-(benzo[d]oxazol-2-ylamino)-4-(2-chlorophenyl)-6-methyl-1,4-dihydropyrimidine-5-carboxamido)methyl)benzoate as starting material. MS m/z (M+H⁺) 502.1, ¹H NMR (400 MHz, DMSO-d₆) δ 10.24 (s, 1H), 9.97 (s, 1H), 8.41 (t, J=6.0 Hz, 1H), 7.52 (dd, J=5.8, 3.4 Hz, 1H), 7.47 (dd, J=5.9, 3.5 Hz, 1H), 7.36 (ddd, J=13.8, 7.7, 2.6 Hz, 4H), 7.19-7.12 (m, 3H), 7.08 (td, J=7.7, 1.3 Hz, 1H), 7.02 (s, 1H), 6.87-6.82 (m, 1H), 6.02 (d, J=2.8 Hz, 1H), 4.38 (s, 2H), 4.26 (d, J=5.9 Hz, 2H), 3.83 (s, 1H), 2.19 (s, 3H).

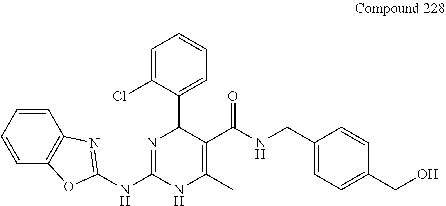 [SMILES: CC1=C(C(=O)NCc2ccc(CO)cc2)C(c2ccccc2Cl)N=C(Nc2nc3ccccc3o2)N1]

The title compound 2-(benzo[d]oxazol-2-ylamino)-4-(2-chlorophenyl)-N-(4-(hydroxymethyl)benzyl)-6-methyl-1,4-dihydropyrimidine-5-carboxamide was prepared according to Example 1b using methyl 4-((2-(benzo[d]oxazol-2-ylamino)-4-(2-chlorophenyl)-6-methyl-1,4-dihydropyrimidine-5-carboxamido)methyl)benzoate as starting material. MS m/z (M+H⁺) 502.1, ¹H NMR (400 MHz, DMSO-d₆) δ 10.21 (s, 1H), 9.97 (s, 1H), 8.38 (t, J=6.0 Hz, 1H), 7.55-7.50 (m, 1H), 7.50-7.45 (m, 1H), 7.38 (ddd, J=7.0, 3.0, 1.9 Hz, 3H), 7.36-7.31 (m, 1H), 7.19-7.11 (m, 3H), 7.11-7.05 (m, 1H), 6.95 (s, 1H), 6.94 (s, 1H), 6.02 (d, J=2.8 Hz, 1H), 4.43 (s, 2H), 4.24 (d, J=6.0 Hz, 3H), 2.18 (s, 3H).

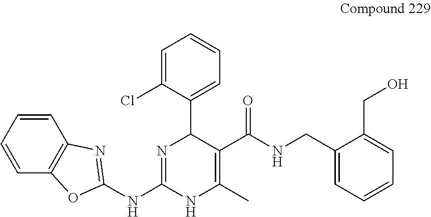 [SMILES: CC1=C(C(=O)NCc2ccccc2CO)C(c2ccccc2Cl)N=C(Nc2nc3ccccc3o2)N1]

The title compound 2-(benzo[d]oxazol-2-ylamino)-4-(2-chlorophenyl)-N-(2-(hydroxymethyl)benzyl)-6-methyl-1,4-dihydropyrimidine-5-carboxamide was prepared according to Example 1b using methyl 2-((2-(benzo[d]oxazol-2-ylamino)-4-(2-chlorophenyl)-6-methyl-1,4-dihydropyrimidine-5-carboxamido)methyl)benzoate as starting material. MS m/z (M+H⁺) 502.1, ¹H NMR (400 MHz, DMSO-d₆) δ 10.21 (s, 1H), 9.98 (s, 1H), 8.29 (t, J=5.8 Hz, 1H), 7.54-7.50 (m, 1H), 7.50-7.46 (m, 1H), 7.38 (dt, J=5.8, 2.3 Hz, 3H), 7.36-7.30 (m, 2H), 7.16 (td, J=7.7, 1.1 Hz, 2H), 7.09 (td, J=7.8, 1.2 Hz, 1H), 7.02 (td, J=7.5, 1.3 Hz, 1H), 6.80 (d, J=7.6 Hz, 1H), 6.03 (d, J=2.7 Hz, 1H), 4.47 (s, 2H), 4.29 (dd, J=5.8, 2.7 Hz, 2H), 2.18 (s, 3H).

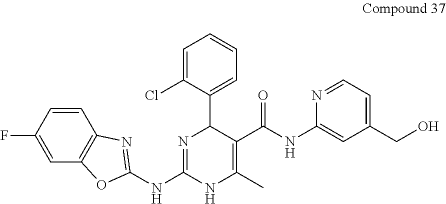 [SMILES: CC1=C(C(=O)Nc2cc(CO)ccn2)C(c2ccccc2Cl)N=C(Nc2nc3ccc(F)cc3o2)N1]

The title compound 4-(2-chlorophenyl)-2-((6-fluorobenzo[d]oxazol-2-yl)amino)-N-(4-(hydroxymethyl)pyridin-2-yl)-6-methyl-1,4-dihydropyrimidine-5-carboxamide was prepared according to Example 1b using methyl 2-(4-(2-chlorophenyl)-2-((6-fluorobenzo[d]oxazol-2-yl)amino)-6-methyl-1,4-dihydropyrimidine-5-carboxamido)isonicotinate as starting material. MS m/z (M+H⁺) 507.0

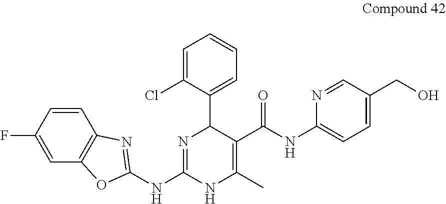 [SMILES: CC1=C(C(=O)Nc2ccc(CO)cn2)C(c2ccccc2Cl)N=C(Nc2nc3ccc(F)cc3o2)N1]

The title compound 4-(2-chlorophenyl)-2-((6-fluorobenzo[d]oxazol-2-yl)amino)-N-(5-(hydroxymethyl)pyridin-2-yl)-6-methyl-1,4-dihydropyrimidine-5-carboxamide was prepared according to Example 1b using methyl 6-(4-(2-chlorophenyl)-2-((6-fluorobenzo[d]oxazol-2-yl)amino)-6-methyl-1,4-dihydropyrimidine-5-carboxamido)nicotinate as starting material. MS m/z (M+H⁺) 507.2

### Example 1c

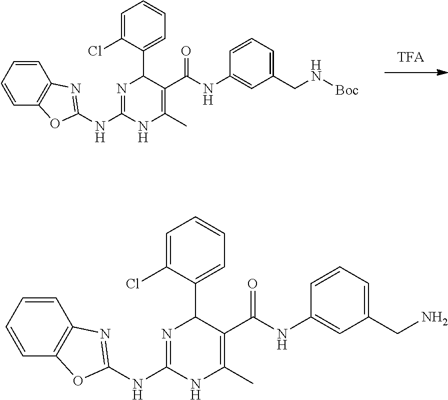 [SMILES: CC1=C(C(=O)Nc2cccc(CNC(=O)OC(C)(C)C)c2)C(c2ccccc2Cl)N=C(Nc2nc3ccccc3o2)N1 ; "TFA" ; CC1=C(C(=O)Nc2cccc(CN)c2)C(c2ccccc2Cl)N=C(Nc2nc3ccccc3o2)N1]

To a solution of tert-butyl 3-(2-(benzo[d]oxazol-2-ylamino)-4-(2-chlorophenyl)-6-methyl-1,4-dihydropyrimidine-5-carboxamido)benzylcarbamate (56 mg, 0.095 mmol) in DCM (2 mL) was added TFA (0.2 mL, 2.6 mmol). The mixture was stirred at R.T. overnight. The solvent was removed. The crude product was purified. (Compound 171). MS m/z (M+H⁺) 487.1, ¹H NMR (400 MHz, DMSO-d₆) δ 10.31 (d, J=2.9 Hz, 1H), 10.16 (d, J=1.8 Hz, 1H), 10.03 (s, 1H), 8.08 (s, 2H), 7.82 (d, J=1.9 Hz, 1H), 7.53 (dd, J=7.7, 1.9 Hz, 1H), 7.49 (dd, J=7.7, 1.5 Hz, 1H), 7.44-7.31 (m, 6H), 7.18 (tt, J=7.7, 1.0 Hz, 1H), 7.14-7.07 (m, 2H), 6.10 (d, J=2.9 Hz, 1H), 3.96 (q, J=5.8 Hz, 2H), 2.23 (s, 3H).

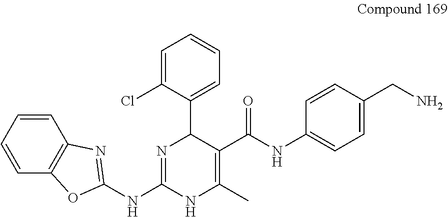 [SMILES: CC1=C(C(=O)Nc2ccc(CN)cc2)C(c2ccccc2Cl)N=C(Nc2nc3ccccc3o2)N1]

The title compound N-(4-(aminomethyl)phenyl)-2-(benzo[d]oxazol-2-ylamino)-4-(2-chlorophenyl)-6-methyl-1,4-dihydropyrimidine-5-carboxamide was prepared according to Example 1c using tert-butyl 4-(2-(benzo[d]oxazol-2-ylamino)-4-(2-chlorophenyl)-6-methyl-1,4-dihydropyrimidine-5-carboxamido)benzylcarbamate as starting material. MS m/z (M+H⁺) 487.1, ¹H NMR (400 MHz, DMSO-d₆) δ 10.28 (t, J=2.3 Hz, 1H), 10.15 (d, J=1.8 Hz, 1H), 9.99 (s, 1H), 8.03 (s, 2H), 7.60-7.55 (m, 2H), 7.50 (ddd, J=13.2, 7.7, 1.7 Hz, 2H), 7.42-7.31 (m, 6H), 7.17 (td, J=7.6, 1.2 Hz, 1H), 7.10 (td, J=7.7, 1.3 Hz, 1H), 6.09 (d, J=2.9 Hz, 1H), 3.95 (q, J=5.7 Hz, 2H), 2.22 (s, 3H).

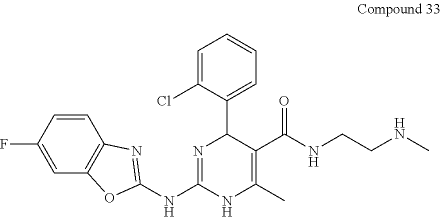 [SMILES: CNCCNC(=O)C1=C(C)NC(Nc2nc3ccc(F)cc3o2)=NC1c1ccccc1Cl]

The title compound 4-(2-chlorophenyl)-2-((6-fluorobenzo[d]oxazol-2-yl)amino)-6-methyl-N-(2-(methylamino)ethyl)-1,4-dihydropyrimidine-5-carboxamide was prepared according to Example 1 using tert-butyl (2-aminoethyl)(methyl)carbamate in Step 1 and using 1-(6-fluorobenzo[d]oxazol-2-yl)guanidine in Step 2, Example 1c using tert-butyl (2-(4-(2-chlorophenyl)-2-((6-fluorobenzo[d]oxazol-2-yl)amino)-6-methyl-1,4-dihydropyrimidine-5-carboxamido)ethyl)(methyl)carbamate as starting material. MS m/z (M+H⁺) 457.1, ¹H NMR (400 MHz, DMSO-d₆) δ 10.21 (d, J=3.0 Hz, 1H), 10.09 (d, J=1.8 Hz, 1H), 8.31 (s, 1H), 8.01 (t, J=5.6 Hz, 1H), 7.50 (ddt, J=7.3, 3.5, 2.0 Hz, 1H), 7.44-7.27 (m, 5H), 7.02 (ddd, J=10.0, 8.6, 2.5 Hz, 1H), 5.91 (d, J=3.0 Hz, 1H), 3.31 (q, J=6.2 Hz, 2H), 2.91 (p, J=6.4 Hz, 2H), 2.54 (t, J=5.5 Hz, 3H), 2.24 (s, 3H).

### Example 1d

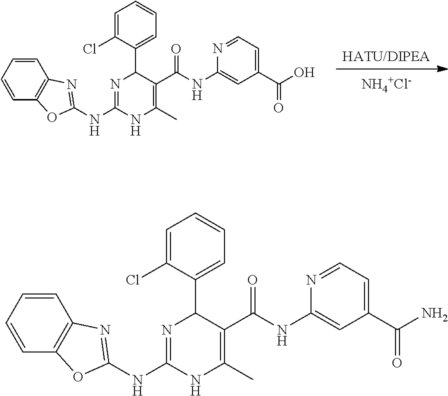 [SMILES: "NH4+Cl-" ; CC1=C(C(=O)Nc2cc(C(=O)O)ccn2)C(c2ccccc2Cl)N=C(Nc2nc3ccccc3o2)N1 ; "HATU/DIPEA" ; CC1=C(C(=O)Nc2cc(C(N)=O)ccn2)C(c2ccccc2Cl)N=C(Nc2nc3ccccc3o2)N1]

A mixture of 2-(2-(benzo[d]oxazol-2-ylamino)-4-(2-chlorophenyl)-6-methyl-1,4-dihydropyrimidine-5-carboxamido)isonicotinic acid (30 mg, 0.060 mmol), HATU (27.2 mg, 0.072 mmol), ammonium chloride (9.57 mg, 0.179 mmol), DOPEA (52.1 μL, 0.298 mmol), and DMF (1.193 mL) was stirred at 23° C. for 1 h. The crude product was purified. (Compound 212). MS m/z (M+H⁺) 502.1

### Example 1e

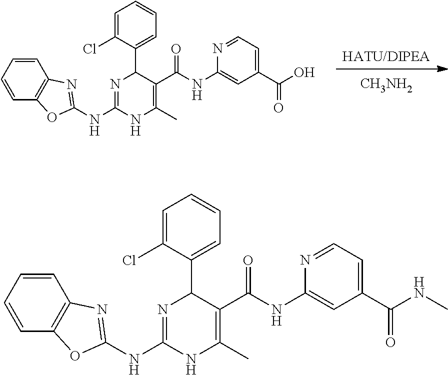 [SMILES: CC1=C(C(=O)Nc2cc(C(=O)O)ccn2)C(c2ccccc2Cl)N=C(Nc2nc3ccccc3o2)N1 ; "CH3NH2" ; "HATU/DIPEA" ; CNC(=O)c1ccnc(NC(=O)C2=C(C)NC(Nc3nc4ccccc4o3)=NC2c2ccccc2Cl)c1]

A mixture of 2-(2-(benzo[d]oxazol-2-ylamino)-4-(2-chlorophenyl)-6-methyl-1,4-dihydropyrimidine-5-carboxamido)isonicotinic acid (30 mg, 0.060 mmol), HATU (27.2 mg, 0.072 mmol), methanamine (59.7 μL, 0.119 mmol), DIPEA (52.1 μL, 0.298 mmol) in DMF (1.193 mL) was stirred at 23° C. for 1 h. The crude product was purified. (Compound 213). MS m/z (M+H⁺) 516.2

### Example 1f

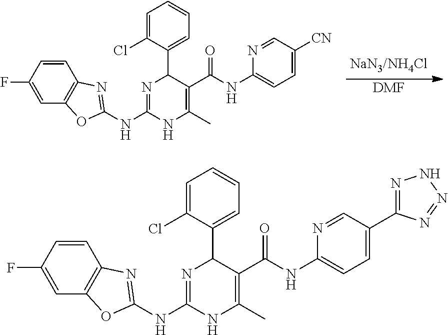

The mixture of 4-(2-chlorophenyl)-N-(5-cyanopyridin-2-yl)-2-((6-fluorobenzo[d]oxazol-2-yl)amino)-6-methyl-1,4-dihydropyrimidine-5-carboxamide (75 mg, 0.149 mmol), SODIUM AZIDE (38.9 mg, 0.598 mmol) and ammonium chloride (32.0 mg, 0.598 mmol) in DMF (Volume: 1.5 mL) was stirred at 120° C. for 1 hr. Water was added to the mixture and extracted with EtOAc (2×). The organic layer was washed with Sat. NaHCO₃ (2×), brine, dried over MgSO₄, and concentrated. The crude product was purified. (Compound 35). MS m/z (M+H⁺) 545.0, ¹H NMR (400 MHz, DMSO-d6) δ 10.78 (s, 1H), 10.22-10.14 (m, 2H), 8.93 (d, J=2.2 Hz, 1H), 8.30 (dd, J=8.8, 2.4 Hz, 1H), 8.13 (d, J=8.7 Hz, 1H), 7.57 (dd, J=7.6, 1.9 Hz, 1H), 7.48 (dd, J=7.6, 1.6 Hz, 1H), 7.41-7.27 (m, 4H), 7.04-6.96 (m, 1H), 6.16 (d, J=2.7 Hz, 1H), 2.26 (s, 3H).

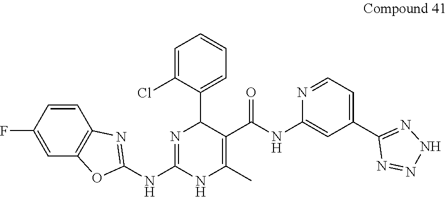 [SMILES: CC1=C(C(=O)Nc2cc(-c3nn[nH]n3)ccn2)C(c2ccccc2Cl)N=C(Nc2nc3ccc(F)cc3o2)N1]

The title compound N-(4-(2H-tetrazol-5-yl)pyridin-2-yl)-4-(2-chlorophenyl)-2-((6-fluorobenzo[d]oxazol-2-yl)amino)-6-methyl-1,4-dihydropyrimidine-5-carboxamide was prepared according to Example 1 using 2-aminoisonicotinonitrile in Step 1 and using 1-(6-fluorobenzo[d]oxazol-2-yl)guanidine in Step 2, Example 1c using 4-(2-chlorophenyl)-N-(4-cyanopyridin-2-yl)-2-((6-fluorobenzo[d]oxazol-2-yl)amino)-6-methyl-1,4-dihydropyrimidine-5-carboxamide as starting material. MS m/z (M+H⁺) 545.0, ¹H NMR (400 MHz, DMSO-d₆) δ 10.72 (s, 1H), 10.19 (d, J=4.1 Hz, 2H), 8.63 (dd, J=1.5, 0.8 Hz, 2H), 8.52 (dd, J=5.1, 0.8 Hz, 1H), 7.66 (dd, J=5.2, 1.5 Hz, 1H), 7.58 (dd, J=7.6, 1.9 Hz, 1H), 7.48 (dd, J=7.8, 1.6 Hz, 1H), 7.41-7.30 (m, 4H), 7.04-6.97 (m, 1H), 6.16 (d, J=2.5 Hz, 1H), 2.27 (s, 3H).

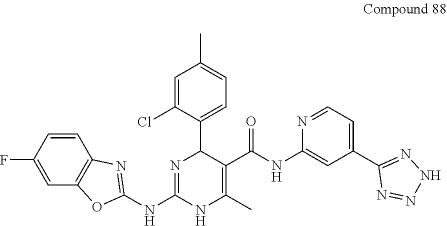 [SMILES: CC1=C(C(=O)Nc2cc(-c3nn[nH]n3)ccn2)C(c2ccc(C)cc2Cl)N=C(Nc2nc3ccc(F)cc3o2)N1]

The title compound N-(4-(2H-tetrazol-5-yl)pyridin-2-yl)-4-(2-chloro-4-methylphenyl)-2-((6-fluorobenzo[d]oxazol-2-yl)amino)-6-methyl-1,4-dihydropyrimidine-5-carboxamide was prepared according to Example 1 using 2-aminoisonicotinonitrile and 2-chloro-5-methylbenzaldehyde in Step 1 and using 1-(6-fluorobenzo[d]oxazol-2-yl)guanidine in Step 2, Example 1c using 4-(2-chloro-4-methylphenyl)-N-(4-cyanopyridin-2-yl)-2-((6-fluorobenzo[d]oxazol-2-yl)amino)-6-methyl-1,4-dihydropyrimidine-5-carboxamide as starting material. MS m/z (M+H⁺) 559.2, ¹H NMR (400 MHz, DMSO-d₆) δ 10.28 (s, 1H), 10.10 (t, J=2.3 Hz, 1H), 10.06 (d, J=1.8 Hz, 1H), 8.51-8.46 (m, 1H), 8.23 (dd, J=5.1, 0.7 Hz, 1H), 7.92 (s, OH), 7.57 (dd, J=5.2, 1.4 Hz, 1H), 7.48 (d, J=7.9 Hz, 1H), 7.36 (dd, J=8.6, 2.5 Hz, 1H), 7.31 (dd, J=8.6, 5.0 Hz, 1H), 7.29-7.28 (m, 2H), 7.15 (dd, J=8.0, 1.6 Hz, 1H), 6.99 (ddd, J=10.2, 8.5, 2.5 Hz, 1H), 6.12 (d, J=2.8 Hz, 1H), 2.23 (s, 3H), 2.22 (s, 3H).

### Example 1g

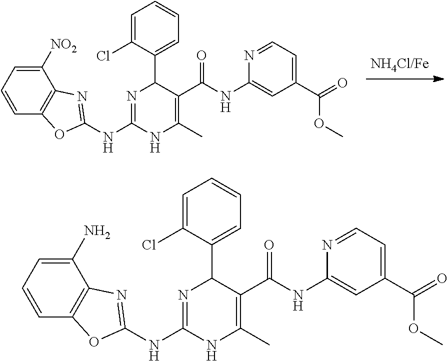 [SMILES: COC(=O)c1ccnc(NC(=O)C2=C(C)NC(Nc3nc4c([N+](=O)[O-])cccc4o3)=NC2c2ccccc2Cl)c1 ; COC(=O)c1ccnc(NC(=O)C2=C(C)NC(Nc3nc4c(N)cccc4o3)=NC2c2ccccc2Cl)c1]

To a suspension of methyl 2-(4-(2-chlorophenyl)-6-methyl-2-((4-nitrobenzo[d]oxazol-2-yl)amino)-1,4-dihydropyrimidine-5-carboxamido)isonicotinate (47 mg, 0.084 mmol) in EtOH (0.4 mL) and Water (0.4 mL) were added ammonium chloride (8.95 mg, 0.167 mmol), followed by iron (23.35 mg, 0.418 mmol). The mixture was stirred at 90° C. for 1.5 hrs. After cooling, EtOAc was added and the reaction mixture was passed through Celite. The solvent was evaporated. The crude product, (methyl 2-(2-((4-aminobenzo[d]oxazol-2-yl)amino)-4-(2-chlorophenyl)-6-methyl-1,4-dihydropyrimidine-5-carboxamido)isonicotinate), was used in the next reaction without purification. (Intermediate to Compound 2)

### Compound 2

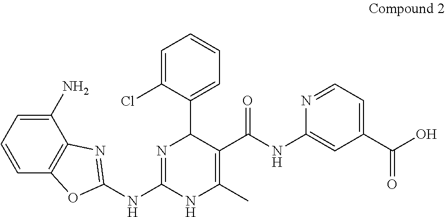 [SMILES: CC1=C(C(=O)Nc2cc(C(=O)O)ccn2)C(c2ccccc2Cl)N=C(Nc2nc3c(N)cccc3o2)N1]

The title compound 2-(2-((4-aminobenzo[d]oxazol-2-yl)amino)-4-(2-chlorophenyl)-6-methyl-1,4-dihydropyrimidine-5-carboxamido)isonicotinic acid was prepared according to Example 1 using methyl 2-aminoisonicotinate in Step 1 and using 1-(4-nitrobenzo[d]oxazol-2-yl)guanidine in Step 2, Example 1f using methyl 2-(4-(2-chlorophenyl)-6-methyl-2-((4-nitrobenzo[d]oxazol-2-yl)amino)-1,4-dihydropyrimidine-5-carboxamido)isonicotinate, Example 1a using methyl 2-(2-((4-aminobenzo[d]oxazol-2-yl)amino)-4-(2-chlorophenyl)-6-methyl-1,4-dihydropyrimidine-5-carboxamido)isonicotinate as starting material. MS m/z (M+H⁺) 518.1, ¹H NMR (400 MHz, DMSO-d₆) δ 13.62 (s, 1H), 10.71 (s, 1H), 10.28 (s, 1H), 10.14 (s, 1H), 8.45 (dd, J=5.1, 0.9 Hz, 1H), 8.42 (dd, J=1.5, 0.9 Hz, 1H), 7.56 (dd, J=7.6, 1.9 Hz, 1H), 7.48 (t, J=1.6 Hz, 1H), 7.46 (dd, J=4.2, 1.5 Hz, 1H), 7.36 (td, J=7.5, 1.6 Hz, 1H), 7.31 (td, J=7.5, 1.9 Hz, 1H), 6.87 (t, J=7.9 Hz, 1H), 6.76 (s, 1H), 6.57 (s, 1H), 6.12-6.06 (m, 1H), 3.85 (s, 2H), 2.23 (d, J=0.8 Hz, 3H).

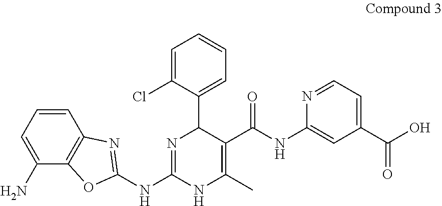 [SMILES: CC1=C(C(=O)Nc2cc(C(=O)O)ccn2)C(c2ccccc2Cl)N=C(Nc2nc3cccc(N)c3o2)N1]

The title compound 2-(2-((7-aminobenzo[d]oxazol-2-yl)amino)-4-(2-chlorophenyl)-6-methyl-1,4-dihydropyrimidine-5-carboxamido)isonicotinic acid was prepared according to Example 1 using methyl 2-aminoisonicotinate in Step 1 and using 1-(7-nitrobenzo[d]oxazol-2-yl)guanidine in Step 2, Example 1f using methyl 2-(4-(2-chlorophenyl)-6-methyl-2-((7-nitrobenzo[d]oxazol-2-yl)amino)-1,4-dihydropyrimidine-5-carboxamido)isonicotinate, Example 1a using methyl 2-(2-((7-aminobenzo[d]oxazol-2-yl)amino)-4-(2-chlorophenyl)-6-methyl-1,4-dihydropyrimidine-5-carboxamido)isonicotinate as starting material. MS m/z (M+H⁺) 518.1, ¹H NMR (400 MHz, DMSO-d₆) δ 13.62 (s, 1H), 10.69 (s, 1H), 10.22 (s, 1H), 10.08 (s, 1H), 8.45 (dd, J=5.1, 0.8 Hz, 1H), 8.40 (dd, J=1.5, 0.9 Hz, 1H), 7.57 (dd, J=7.6, 1.8 Hz, 1H), 7.48 (d, J=1.5 Hz, 1H), 7.46 (dd, J=3.2, 1.5 Hz, 1H), 7.37 (td, J=7.5, 1.5 Hz, 1H), 7.31 (td, J=7.6, 1.9 Hz, 1H), 6.89 (t, J=7.9 Hz, 1H), 6.67-6.60 (m, 1H), 6.50-6.43 (m, 1H), 6.15-6.10 (m, 1H), 3.99 (s, 2H), 2.34-2.09 (m, 3H).

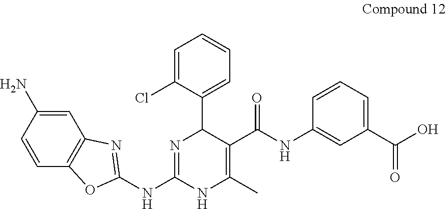 [SMILES: CC1=C(C(=O)Nc2cccc(C(=O)O)c2)C(c2ccccc2Cl)N=C(Nc2nc3cc(N)ccc3o2)N1]

The title compound 2-(2-((5-aminobenzo[d]oxazol-2-yl)amino)-4-(2-chlorophenyl)-6-methyl-1,4-dihydropyrimidine-5-carboxamido)isonicotinic acid was prepared according to Example 1 using methyl 2-aminoisonicotinate in Step 1 and using 1-(5-nitrobenzo[d]oxazol-2-yl)guanidine in Step 2, Example 1f using methyl 2-(4-(2-chlorophenyl)-6-methyl-2-((5-nitrobenzo[d]oxazol-2-yl)amino)-1,4-dihydropyrimidine-5-carboxamido)isonicotinate, Example 1a using methyl 2-(2-((5-aminobenzo[d]oxazol-2-yl)amino)-4-(2-chlorophenyl)-6-methyl-1,4-dihydropyrimidine-5-carboxamido)isonicotinate as starting material. MS m/z (M+H⁺) 518.1, ¹H NMR (400 MHz, DMSO-d₆) δ 10.66 (s, 1H), 10.28 (s, 1H), 10.18 (s, 1H), 8.45 (dd, J=5.1, 0.9 Hz, 1H), 8.40 (t, J=1.2 Hz, 1H), 7.58 (dd, J=7.6, 1.9 Hz, 1H), 7.48 (t, J=1.7 Hz, 1H), 7.47 (t, J=1.2 Hz, 1H), 7.42-7.27 (m, 3H), 7.10 (s, 1H), 6.84 (s, 1H), 6.13 (d, J=2.9 Hz, 1H), 2.25 (s, 3H).

### Example 1h

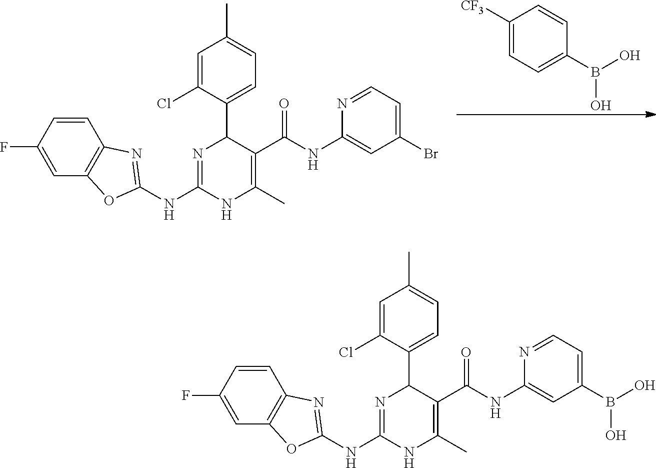 [SMILES: CC1=C(C(=O)Nc2cc(Br)ccn2)C(c2ccc(C)cc2Cl)N=C(Nc2nc3ccc(F)cc3o2)N1 ; OB(O)c1ccc(C(F)(F)F)cc1 ; CC1=C(C(=O)Nc2cc(B(O)O)ccn2)C(c2ccc(C)cc2Cl)N=C(Nc2nc3ccc(F)cc3o2)N1]

A mixture of N-(4-bromopyridin-2-yl)-4-(2-chloro-4-methylphenyl)-2-((6-fluorobenzo[d]oxazol-2-yl)amino)-6-methyl-1,4-dihydropyrimidine-5-carboxamide (60 mg, 0.105 mmol), bis(pinacolato)diboron (34.8 mg, 0.137 mmol), 1,1′-bis(diphenylphosphino)ferrocene dichloro palladium(II) dichloromethane complex (7.70 mg, 10.53 μmol), and potassium acetate (31.0 mg, 0.316 mmol) in Dioxane (1 mL) was heated to 95° C. overnight. The reaction mixture was allowed to cool to R.T., and then partitioned between EtOAc and H₂O. The biphasic mixture was filtered through Celite and the filter cake rinsed with EtOAc, the filtrate was separated, and the organic phase washed with brine (2×), dried over MgSO₄, filtered and concentrated. The crude product was purified by ISCO (10-100%, ACN/H₂O, 0.1% TFA). (Compound 92). ¹H NMR (400 MHz, DMSO-d₆) δ 10.33 (s, 1H), 10.11 (s, 1H), 10.08 (s, 1H), 8.43 (s, 1H), 8.37 (s, 1H), 8.24 (d, J=5.5 Hz, 1H), 8.16 (s, 1H), 7.44 (d, J=7.9 Hz, 1H), 7.39-7.27 (m, 4H), 7.14 (d, J=7.9 Hz, 1H), 7.04-6.92 (m, 1H), 6.08 (s, 1H), 2.22 (s, 6H).

### Example 2

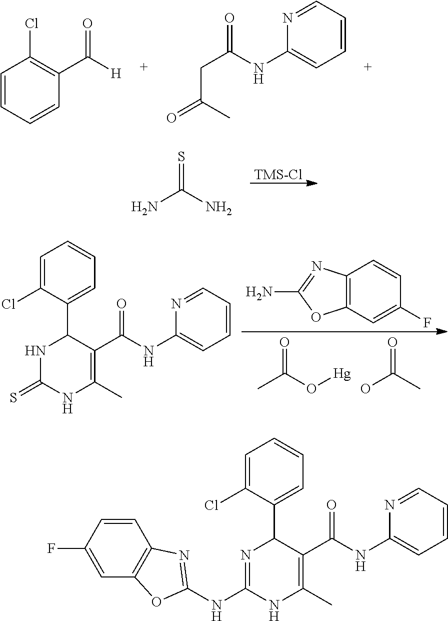

Step 1. To a solution of 3-oxo-N-(pyridin-2-yl)butanamide (0.4 g, 2.245 mmol), 2-chlorobenzaldehyde (0.253 mL, 2.245 mmol) and thiourea (0.171 g, 2.245 mmol) in Acetonitrile (6 mL) and DMF (3.00 mL) was added dropwise TMS-cl (0.287 mL, 2.245 mmol) at R.T. The mixture was stirred at 80° C. overnight. The reaction mixture was poured onto crushed ice and stirred until all ice had melted. The solid was filtered and dried. The crude product was used in the next reaction without further purification (0.51 g, 63.3%). MS m/z (M+H⁺) 359.1.

Step 2. The mixture of 4-(2-chlorophenyl)-6-methyl-N-(pyridin-2-yl)-2-thioxo-1,2,3,4-tetrahydropyrimidine-5-carboxamide (0.2 g, 0.557 mmol), 6-fluorobenzo[d]oxazol-2-amine (0.127 g, 0.836 mmol) and mercuric acetate (0.213 g, 0.669 mmol) in DCM (5 mL) and DMF (1.000 mL) in the sealed tube was stirred at 80° C. for 72 hr. The reaction was diluted with EtOAc and filtered through Celite and washed with EtOAc. The filtrate was concentrated. The crude product was purified by ISCO (30-100%, EtOAc/hex). MS m/z (M+H⁺) 477.2, ¹H NMR (400 MHz, DMSO-d₆) δ 10.52 (s, 1H), 10.16 (s, 2H), 8.28 (ddd, J=5.0, 1.9, 0.9 Hz, 1H), 7.87 (dt, J=8.5, 0.9 Hz, 1H), 7.78-7.71 (m, 1H), 7.56 (dd, J=7.6, 1.9 Hz, 1H), 7.47 (dd, J=7.7, 1.5 Hz, 1H), 7.41-7.26 (m, 4H), 7.08 (ddd, J=7.3, 5.0, 1.0 Hz, 1H), 7.00 (ddd, J=10.2, 8.6, 2.6 Hz, 1H), 6.11 (d, J=2.6 Hz, 1H), 2.23 (s, 3H). (Compound 43)

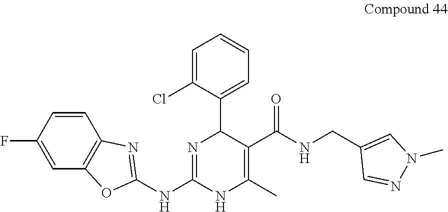 [SMILES: CC1=C(C(=O)NCc2cnn(C)c2)C(c2ccccc2Cl)N=C(Nc2nc3ccc(F)cc3o2)N1]

The title compound 4-(2-chlorophenyl)-2-((6-fluorobenzo[d]oxazol-2-yl)amino)-6-methyl-N-((1-methyl-1H-pyrazol-4-yl)methyl)-1,4-dihydropyrimidine-5-carboxamide was prepared according to Example 2 using N-((1-methyl-1H-pyrazol-4-yl)methyl)-3-oxobutanamide in Step 1 as starting material. MS m/z (M+H⁺) 494.2, ¹H NMR (400 MHz, DMSO-d₆) δ 10.06 (t, J=2.3 Hz, 1H), 9.92 (d, J=1.8 Hz, 1H), 8.18 (t, J=5.7 Hz, 1H), 7.51-7.43 (m, 1H), 7.40 (dd, J=5.9, 3.5 Hz, 1H), 7.37-7.26 (m, 4H), 7.24 (s, 1H), 7.09 (s, 1H), 6.97 (ddd, J=10.2, 8.6, 2.5 Hz, 1H), 5.93 (d, J=2.8 Hz, 1H), 4.03 (d, J=5.7 Hz, 2H), 3.69 (s, 3H), 2.12 (s, 3H).

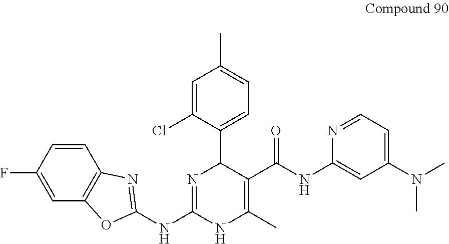 [SMILES: CC1=C(C(=O)Nc2cc(N(C)C)ccn2)C(c2ccc(C)cc2Cl)N=C(Nc2nc3ccc(F)cc3o2)N1]

The title compound 4-(2-chloro-4-methylphenyl)-N-(4-(dimethylamino)pyridin-2-yl)-2-((6-fluorobenzo[d]oxazol-2-yl)amino)-6-methyl-1,4-dihydropyrimidine-5-carboxamide was prepared according to Example 2 using N-(4-(dimethylamino)pyridin-2-yl)-3-oxobutanamide and 2-chloro-4-methylbenzaldehyde in Step 1, and 6-fluorobenzo[d]oxazol-2-amine in Step 2 as starting material. MS m/z (M+H⁺) 534.3, ¹H NMR (400 MHz, DMSO-d₆) δ 10.10 (s, 1H), 10.01 (s, 1H), 9.93 (s, 1H), 7.92 (s, 1H), 7.82 (d, J=6.0 Hz, 1H), 7.47 (d, J=7.9 Hz, 1H), 7.35 (dd, J=8.6, 2.6 Hz, 1H), 7.30 (q, J=4.8 Hz, 2H), 7.14 (d, J=8.0 Hz, 1H), 7.04-6.93 (m, 1H), 6.33 (dd, J=6.2, 2.4 Hz, 1H), 6.04 (d, J=2.6 Hz, 1H), 2.88 (s, 6H), 2.22 (s, 3H), 2.20 (s, 3H).

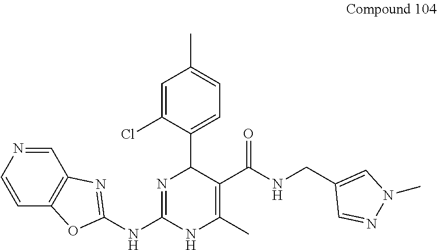 [SMILES: CC1=C(C(=O)NCc2cnn(C)c2)C(c2ccc(C)cc2Cl)N=C(Nc2nc3cnccc3o2)N1]

The title compound 4-(2-chloro-4-methylphenyl)-6-methyl-N-((1-methyl-1H-pyrazol-4-yl)methyl)-2-(oxazolo[4,5-c]pyridin-2-ylamino)-1,4-dihydropyrimidine-5-carboxamide was prepared according to Example 2 using N-((1-methyl-1H-pyrazol-4-yl)methyl)-3-oxobutanamide and 2-chloro-4-methylbenzaldehyde in Step 1, and using oxazolo[4,5-c]pyridin-2-amine in Step 2 as starting material. MS m/z (M+H⁺) 491.2, ¹H NMR (400 MHz, DMSO-d₆) δ 10.18 (s, 1H), 9.80 (t, J=2.3 Hz, 1H), 8.87 (s, 1H), 8.51 (d, J=6.2 Hz, 1H), 8.23 (t, J=5.7 Hz, 1H), 7.89 (d, J=6.0 Hz, 1H), 7.33-7.27 (m, 2H), 7.25 (s, 1H), 7.14 (d, J=8.2 Hz, 1H), 7.09 (s, 1H), 5.96 (d, J=2.6 Hz, 1H), 4.03 (d, J=5.9 Hz, 2H), 3.69 (s, 3H), 2.26 (s, 3H), 2.10 (s, 3H).

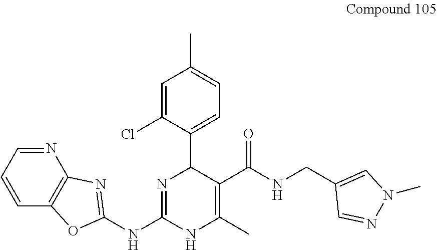 [SMILES: CC1=C(C(=O)NCc2cnn(C)c2)C(c2ccc(C)cc2Cl)N=C(Nc2nc3ncccc3o2)N1]

The title compound 4-(2-chloro-4-methylphenyl)-6-methyl-N-((1-methyl-1H-pyrazol-4-yl)methyl)-2-(oxazolo[4,5-b]pyridin-2-ylamino)-1,4-dihydropyrimidine-5-carboxamide was prepared according to Example 2 using N-((1-methyl-1H-pyrazol-4-yl)methyl)-3-oxobutanamide and 2-chloro-4-methylbenzaldehyde in Step 1, and using oxazolo[4,5-b]pyridin-2-amine in Step 2 as starting material. MS m/z (M+H⁺) 491.2, ¹H NMR (400 MHz, DMSO-d₆) δ 10.07 (s, 1H), 9.94 (t, J=2.3 Hz, 1H), 8.20 (t, J=5.7 Hz, 1H), 8.14 (d, J=4.9 Hz, 1H), 7.74 (d, J=7.8 Hz, 1H), 7.33-7.28 (m, 2H), 7.23 (s, 1H), 7.13 (d, J=8.6 Hz, 1H), 7.10 (s, 1H), 7.07 (dd, J=7.9, 5.2 Hz, 1H), 5.95 (d, J=2.7 Hz, 1H), 4.03 (dd, J=5.7, 2.9 Hz, 2H), 3.69 (s, 3H), 2.25 (s, 3H), 2.10 (s, 3H).

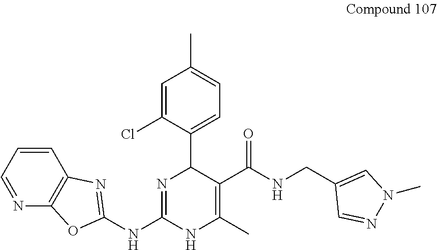 [SMILES: CC1=C(C(=O)NCc2cnn(C)c2)C(c2ccc(C)cc2Cl)N=C(Nc2nc3cccnc3o2)N1]

The title compound 4-(2-chloro-4-methylphenyl)-6-methyl-N-((1-methyl-1H-pyrazol-4-yl)methyl)-2-(oxazolo[5,4-b]pyridin-2-ylamino)-1,4-dihydropyrimidine-5-carboxamide was prepared according to Example 2 using N-((1-methyl-1H-pyrazol-4-yl)methyl)-3-oxobutanamide and 2-chloro-4-methylbenzaldehyde in Step 1, and using oxazolo[5,4-b]pyridin-2-amine in Step 2 as starting material. MS m/z (M+H⁺) 491.2, ¹H NMR (400 MHz, DMSO-d₆) δ 10.00 (s, 2H), 8.17 (t, J=5.7 Hz, 1H), 7.93 (dd, J=5.1, 1.5 Hz, 1H), 7.65 (dd, J=7.7, 1.5 Hz, 1H), 7.29 (d, J=8.0 Hz, 2H), 7.24 (s, 1H), 7.21-7.11 (m, 2H), 7.10 (s, 1H), 5.91 (s, 1H), 4.05-4.01 (m, 2H), 3.69 (s, 3H), 2.25 (s, 3H), 2.10 (s, 3H).

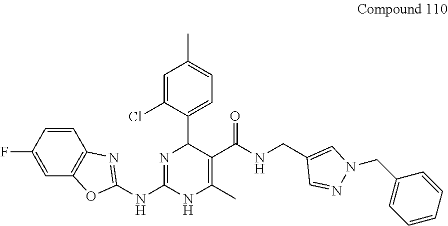 [SMILES: CC1=C(C(=O)NCc2cnn(Cc3ccccc3)c2)C(c2ccc(C)cc2Cl)N=C(Nc2nc3ccc(F)cc3o2)N1]

The title compound N-((1-benzyl-1H-pyrazol-4-yl)methyl)-4-(2-chloro-4-methylphenyl)-2-((6-fluorobenzo[d]oxazol-2-yl)amino)-6-methyl-1,4-dihydropyrimidine-5-carboxamide was prepared according to Example 2 using N-((1-benzyl-1H-pyrazol-4-yl)methyl)-3-oxobutanamide and 2-chloro-4-methylbenzaldehyde in Step 1, and using 6-fluorobenzo[d]oxazol-2-amine in Step 2 as starting material. MS m/z (M+H⁺) 584.2, ¹H NMR (400 MHz, DMSO-d₆) δ 10.03 (s, 1H), 9.87 (d, J=1.8 Hz, 1H), 8.18 (t, J=5.7 Hz, 1H), 7.41 (s, 1H), 7.37-7.20 (m, 7H), 7.20-7.04 (m, 4H), 6.97 (ddd, J=10.2, 8.6, 2.5 Hz, 1H), 5.92-5.86 (m, 1H), 5.18 (s, 2H), 4.08-4.02 (m, 2H), 2.22 (s, 3H), 2.12-2.07 (m, 3H).

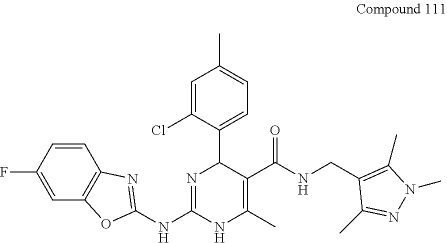 [SMILES: CC1=C(C(=O)NCc2c(C)nn(C)c2C)C(c2ccc(C)cc2Cl)N=C(Nc2nc3ccc(F)cc3o2)N1]

The title compound 4-(2-chloro-4-methylphenyl)-2-((6-fluorobenzo[d]oxazol-2-yl)amino)-6-methyl-N-((1,3,5-trimethyl-1H-pyrazol-4-yl)methyl)-1,4-dihydropyrimidine-5-carboxamide was prepared according to Example 2 using 3-oxo-N-((1,3,5-trimethyl-1H-pyrazol-4-yl)methyl)butanamide and 2-chloro-4-methylbenzaldehyde in Step 1, and using 6-fluorobenzo[d]oxazol-2-amine in Step 2 as starting material. MS m/z (M+H⁺) 536.2, ¹H NMR (400 MHz, DMSO-d₆) δ 10.01 (t, J=2.3 Hz, 1H), 9.83 (d, J=1.8 Hz, 1H), 7.94 (t, J=5.3 Hz, 1H), 7.33 (dd, J=8.6, 2.5 Hz, 1H), 7.31-7.22 (m, 3H), 7.08 (dd, J=7.9, 1.3 Hz, 1H), 6.97 (ddd, J=10.3, 8.6, 2.6 Hz, 1H), 5.87 (d, J=3.3 Hz, 1H), 3.91 (t, J=5.6 Hz, 2H), 3.54 (s, 3H), 2.24 (s, 3H), 2.06 (s, 3H), 2.01 (s, 3H), 1.90 (s, 3H).

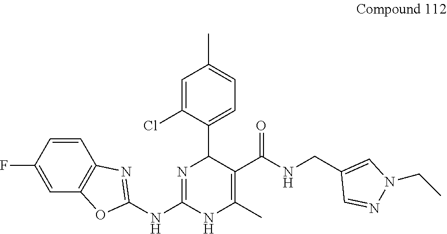 [SMILES: CCn1cc(CNC(=O)C2=C(C)NC(Nc3nc4ccc(F)cc4o3)=NC2c2ccc(C)cc2Cl)cn1]

The title compound 4-(2-chloro-4-methylphenyl)-N-((1-ethyl-1H-pyrazol-4-yl)methyl)-2-((6-fluorobenzo[d]oxazol-2-yl)amino)-6-methyl-1,4-dihydropyrimidine-5-carboxamide was prepared according to Example 2 using 3 N-((1-ethyl-1H-pyrazol-4-yl)methyl)-3-oxobutanamide and 2-chloro-4-methylbenzaldehyde in Step 1, and using 6-fluorobenzo[d]oxazol-2-amine in Step 2 as starting material. MS m/z (M+H⁺) 522.2, ¹H NMR (400 MHz, DMSO-d₆) δ 10.03 (t, J=2.3 Hz, 1H), 9.87 (d, J=1.8 Hz, 1H), 8.16 (t, J=5.7 Hz, 1H), 7.34 (dd, J=8.6, 2.5 Hz, 1H), 7.32-7.24 (m, 4H), 7.14-7.09 (m, 2H), 6.97 (ddd, J=10.2, 8.6, 2.6 Hz, 1H), 5.93-5.87 (m, 1H), 4.04 (dd, J=5.6, 3.0 Hz, 2H), 3.97 (q, J=7.3 Hz, 2H), 2.24 (s, 3H), 2.11 (s, 3H), 1.25 (t, J=7.3 Hz, 3H).

### Example 2a

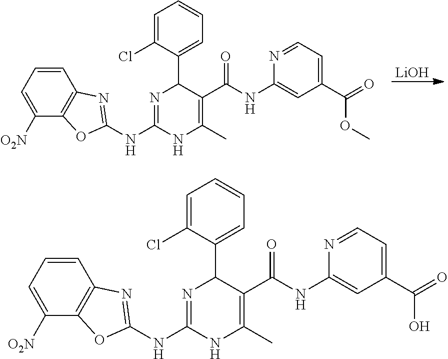

To a solution of methyl 2-(4-(2-chlorophenyl)-6-methyl-2-((7-nitrobenzo[d]oxazol-2-yl)amino)-1,4-dihydropyrimidine-5-carboxamido)isonicotinate (25 mg, 0.044 mmol) in THF (0.4 mL) was added LiOH (0.178 mL, 0.089 mmol)(0.5M). The mixture was stirred at R.T. for 2 hr. After reaction was completed, the solvent was removed. 1 N HCl was added to the residue. The solid was filtered and washed with water and dried. MS m/z (M+H⁺) 548.1, ¹H NMR (400 MHz, DMSO-d₆) δ 13.55 (s, 1H), 10.72 (s, 1H), 10.28 (d, J=1.8 Hz, 1H), 10.00 (t, J=2.5 Hz, 1H), 8.45 (dd, J=5.1, 0.8 Hz, 1H), 8.40 (dd, J=1.5, 0.9 Hz, 1H), 7.85 (dd, J=8.5, 1.0 Hz, 1H), 7.75 (dd, J=7.8, 1.0 Hz, 1H), 7.59 (dd, J=7.6, 1.9 Hz, 1H), 7.49 (d, J=1.5 Hz, 1H), 7.47 (dd, J=2.7, 1.5 Hz, 1H), 7.40-7.27 (m, 3H), 6.18 (d, J=2.9 Hz, 1H), 2.27-2.22 (m, 3H). (Compound 23) Compound 60

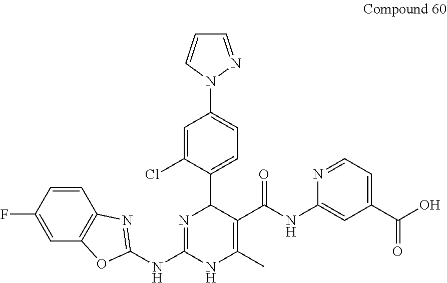 [SMILES: CC1=C(C(=O)Nc2cc(C(=O)O)ccn2)C(c2ccc(-n3cccn3)cc2Cl)N=C(Nc2nc3ccc(F)cc3o2)N1]

The title compound 2-(4-(2-chloro-4-(1H-pyrazol-1-yl)phenyl)-2-((6-fluorobenzo[d]oxazol-2-yl)amino)-6-methyl-1,4-dihydropyrimidine-5-carboxamido)isonicotinic acid was prepared according to Example 2 using methyl 2-(3-oxobutanamido)isonicotinate and 2-chloro-4-(1H-pyrazol-1-yl)benzaldehyde in Step 1, Example 2a using methyl 2-(4-(2-chloro-4-(1H-pyrazol-1-yl)phenyl)-2-((6-fluorobenzo[d]oxazol-2-yl)amino)-6-methyl-1,4-dihydropyrimidine-5-carboxamido)isonicotinate as starting material. MS m/z (M+H⁺) 587.2, ¹H NMR (400 MHz, DMSO-d₆) δ 13.56 (s, 1H), 10.67 (s, 1H), 10.19 (s, 1H), 10.15 (s, 1H), 8.52 (d, J=2.6 Hz, 1H), 8.45 (d, J=5.1 Hz, 1H), 8.42-8.40 (m, 1H), 7.97 (d, J=2.6 Hz, 1H), 7.81 (dd, J=8.6, 2.4 Hz, 1H), 7.72 (d, J=1.6 Hz, 1H), 7.65 (d, J=8.5 Hz, 1H), 7.50-7.44 (m, 1H), 7.38 (dd, J=8.5, 2.6 Hz, 1H), 7.34 (dd, J=8.7, 4.9 Hz, 1H), 7.05-6.95 (m, 1H), 6.51 (t, J=1.7 Hz, 1H), 6.15 (d, J=2.9 Hz, 1H), 2.25 (s, 3H).

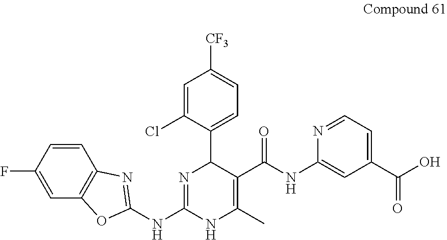 [SMILES: CC1=C(C(=O)Nc2cc(C(=O)O)ccn2)C(c2ccc(C(F)(F)F)cc2Cl)N=C(Nc2nc3ccc(F)cc3o2)N1]

The title compound 2-(4-(2-chloro-4-(trifluoromethyl)phenyl)-2-((6-fluorobenzo[d]oxazol-2-yl)amino)-6-methyl-1,4-dihydropyrimidine-5-carboxamido)isonicotinic acid was prepared according to Example 2 using methyl 2-(3-oxobutanamido)isonicotinate and 2-chloro-4-(trifluoromethyl)benzaldehyde in Step 1, Example 2a using methyl 2-(4-(2-chloro-4-(trifluoromethyl)phenyl)-2-((6-fluorobenzo[d]oxazol-2-yl)amino)-6-methyl-1,4-dihydropyrimidine-5-carboxamido)isonicotinate as starting material. MS m/z (M+H⁺) 589.2, ¹H NMR (400 MHz, DMSO-d₆) δ 13.58 (s, 1H), 10.70 (s, 1H), 10.25 (s, 1H), 10.18 (d, J=3.3 Hz, 1H), 8.45 (d, J=5.0 Hz, 1H), 8.40-8.38 (m, 1H), 7.90 (s, 1H), 7.74 (s, 2H), 7.48 (dd, J=5.0, 0.9 Hz, 1H), 7.39 (dd, J=8.6, 2.6 Hz, 1H), 7.34 (dd, J=8.6, 5.0 Hz, 1H), 7.06-6.96 (m, 1H), 6.16 (d, J=3.1 Hz, 1H), 2.24 (s, 3H).

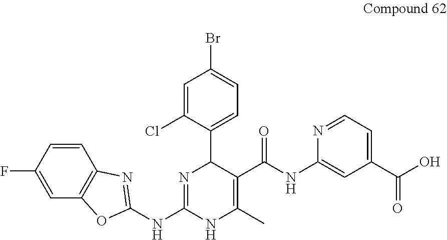 [SMILES: CC1=C(C(=O)Nc2cc(C(=O)O)ccn2)C(c2ccc(Br)cc2Cl)N=C(Nc2nc3ccc(F)cc3o2)N1]

The title compound 2-(4-(4-bromo-2-chlorophenyl)-2-((6-fluorobenzo[d]oxazol-2-yl)amino)-6-methyl-1,4-dihydropyrimidine-5-carboxamido)isonicotinic acid was prepared according to Example 2 using methyl 2-(3-oxobutanamido)isonicotinate and 4-bromo-2-chlorobenzaldehyde in Step 1, Example 2a using methyl 2-(4-(4-bromo-2-chlorophenyl)-2-((6-fluorobenzo[d]oxazol-2-yl)amino)-6-methyl-1,4-dihydropyrimidine-5-carboxamido)isonicotinate as starting material. MS m/z (M+H⁺) 599.1/601.1, ¹H NMR (400 MHz, DMSO-d₆) δ 13.55 (s, 1H), 10.67 (s, 1H), 10.20 (s, 1H), 10.13 (s, 1H), 8.45 (d, J=5.1 Hz, 1H), 8.41-8.39 (m, 1H), 7.76 (dd, J=2.0, 0.5 Hz, 1H), 7.57 (dd, J=8.4, 2.0 Hz, 1H), 7.52-7.45 (m, 2H), 7.38 (dd, J=8.6, 2.6 Hz, 1H), 7.33 (dd, J=8.6, 5.0 Hz, 1H), 7.01 (ddd, J=10.7, 9.1, 2.6 Hz, 1H), 6.08 (d, J=2.9 Hz, 1H), 2.22 (s, 3H).

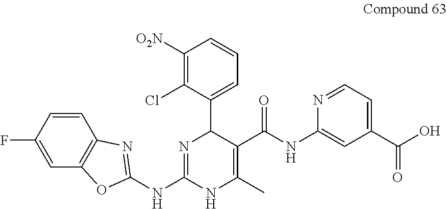 [SMILES: CC1=C(C(=O)Nc2cc(C(=O)O)ccn2)C(c2cccc([N+](=O)[O-])c2Cl)N=C(Nc2nc3ccc(F)cc3o2)N1]

The title compound 2-(4-(2-chloro-3-nitrophenyl)-2-((6-fluorobenzo[d]oxazol-2-yl)amino)-6-methyl-1,4-dihydropyrimidine-5-carboxamido)isonicotinic acid was prepared according to Example 2 using methyl 2-(3-oxobutanamido)isonicotinate and 2-chloro-3-nitrobenzaldehyde in Step 1, Example 2a using methyl 2-(4-(2-chloro-3-nitrophenyl)-2-((6-fluorobenzo[d]oxazol-2-yl)amino)-6-methyl-1,4-dihydropyrimidine-5-carboxamido)isonicotinate as starting material. MS m/z (M+H⁺) 566.2, ¹H NMR (400 MHz, DMSO-d₆) δ 13.58 (s, 1H), 10.68 (s, 1H), 10.28 (d, J=1.8 Hz, 1H), 10.16 (t, J=2.5 Hz, 1H), 8.45 (dd, J=5.1, 0.8 Hz, 1H), 8.41-8.35 (m, 1H), 7.99-7.91 (m, 1H), 7.86-7.78 (m, 1H), 7.61 (t, J=7.9 Hz, 1H), 7.47 (dd, J=5.1, 1.5 Hz, 1H), 7.38 (dd, J=8.6, 2.5 Hz, 1H), 7.34 (dd, J=8.7, 5.0 Hz, 1H), 7.07-6.95 (m, 1H), 6.19 (d, J=3.1 Hz, 1H), 2.24 (s, 3H).

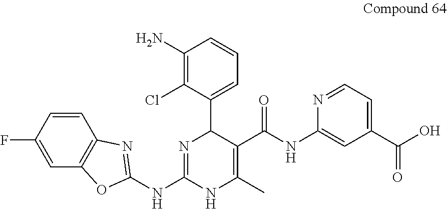 [SMILES: CC1=C(C(=O)Nc2cc(C(=O)O)ccn2)C(c2cccc(N)c2Cl)N=C(Nc2nc3ccc(F)cc3o2)N1]

The title compound 2-(4-(3-amino-2-chlorophenyl)-2-((6-fluorobenzo[d]oxazol-2-yl)amino)-6-methyl-1,4-dihydropyrimidine-5-carboxamido)isonicotinic acid was prepared according to Example 2 using methyl 2-(3-oxobutanamido)isonicotinate and 2-chloro-3-nitrobenzaldehyde in Step 1, Example 2b using methyl 2-(4-(2-chloro-3-nitrophenyl)-2-((6-fluorobenzo[d]oxazol-2-yl)amino)-6-methyl-1,4-dihydropyrimidine-5-carboxamido)isonicotinate, Example 2a using methyl 2-(4-(3-amino-2-chlorophenyl)-2-((6-fluorobenzo[d]oxazol-2-yl)amino)-6-methyl-1,4-dihydropyrimidine-5-carboxamido)isonicotinate as starting material. MS m/z (M+H⁺) 536.2, ¹H NMR (400 MHz, DMSO-d₆) δ 13.56 (s, 1H), 10.54 (s, 1H), 10.14 (t, J=2.4 Hz, 1H), 10.06 (d, J=1.8 Hz, 1H), 8.43 (dd, J=5.1, 0.8 Hz, 1H), 8.41 (dd, J=1.5, 0.9 Hz, 1H), 7.46 (dd, J=5.0, 1.6 Hz, 1H), 7.37 (dd, J=8.5, 2.5 Hz, 1H), 7.30 (dd, J=8.6, 5.0 Hz, 1H), 7.04-6.95 (m, 2H), 6.77-6.67 (m, 2H), 6.08 (d, J=2.8 Hz, 1H), 5.34 (s, 2H), 2.21 (s, 3H).

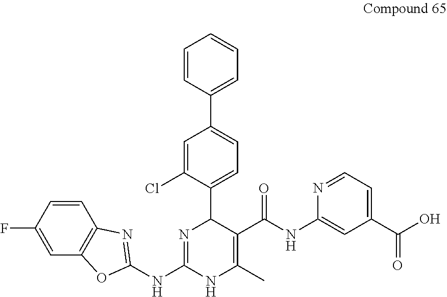 [SMILES: CC1=C(C(=O)Nc2cc(C(=O)O)ccn2)C(c2ccc(-c3ccccc3)cc2Cl)N=C(Nc2nc3ccc(F)cc3o2)N1]

The title compound 2-(4-(3-chloro-[1,1′-biphenyl]-4-yl)-2-((6-fluorobenzo[d]oxazol-2-yl)amino)-6-methyl-1,4-dihydropyrimidine-5-carboxamido)isonicotinic acid was prepared according to Example 2 using methyl 2-(3-oxobutanamido)isonicotinate and 3-chloro-[1,1′-biphenyl]-4-carbaldehyde in Step 1, Example 2a using methyl 2-(4-(3-chloro-[1,1′-biphenyl]-4-yl)-2-((6-fluorobenzo[d]oxazol-2-yl)amino)-6-methyl-1,4-dihydropyrimidine-5-carboxamido)isonicotinate as starting material. MS m/z (M+H⁺) 597.2, ¹H NMR (400 MHz, DMSO-d₆) δ 13.58 (s, 1H), 10.64 (s, 1H), 10.18 (d, J=3.7 Hz, 2H), 8.41 (d, J=8.8 Hz, 2H), 7.77-7.71 (m, 1H), 7.65 (d, J=1.6 Hz, 1H), 7.62 (d, J=1.3 Hz, 3H), 7.48-7.29 (m, 6H), 7.05-6.95 (m, 1H), 6.16 (d, J=2.6 Hz, 1H), 2.25 (s, 3H).

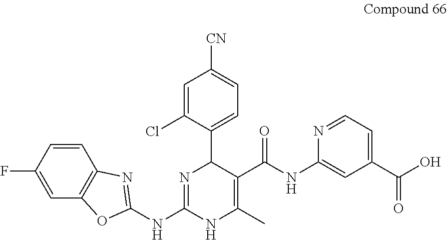 [SMILES: CC1=C(C(=O)Nc2cc(C(=O)O)ccn2)C(c2ccc(C#N)cc2Cl)N=C(Nc2nc3ccc(F)cc3o2)N1]

The title compound 2-(4-(2-chloro-4-cyanophenyl)-2-((6-fluorobenzo[d]oxazol-2-yl)amino)-6-methyl-1,4-dihydropyrimidine-5-carboxamido)isonicotinic acid was prepared according to Example 2 using methyl 2-(3-oxobutanamido)isonicotinate and 3-chloro-4-formylbenzonitrile in Step 1, Example 2a using methyl 2-(4-(2-chloro-4-cyanophenyl)-2-((6-fluorobenzo[d]oxazol-2-yl)amino)-6-methyl-1,4-dihydropyrimidine-5-carboxamido)isonicotinate as starting material. MS m/z (M+H⁺) 546.2, ¹H NMR (400 MHz, DMSO-d₆) δ 13.60 (s, 1H), 10.70 (s, 1H), 10.26 (s, 1H), 10.18 (d, J=3.2 Hz, 1H), 8.45 (dd, J=5.1, 0.7 Hz, 1H), 8.41-8.36 (m, 1H), 8.08 (d, J=1.6 Hz, 1H), 7.85 (dd, J=8.1, 1.6 Hz, 1H), 7.69 (d, J=8.1 Hz, 1H), 7.47 (dd, J=5.1, 1.4 Hz, 1H), 7.39 (dd, J=8.6, 2.5 Hz, 1H), 7.34 (dd, J=8.7, 5.0 Hz, 1H), 7.06-6.96 (m, 1H), 6.12 (d, J=3.1 Hz, 1H), 2.23 (s, 3H).

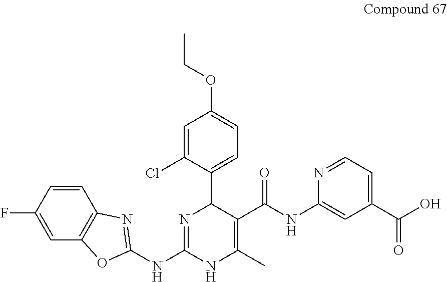 [SMILES: CCOc1ccc(C2N=C(Nc3nc4ccc(F)cc4o3)NC(C)=C2C(=O)Nc2cc(C(=O)O)ccn2)c(Cl)c1]

The title compound 2-(4-(2-chloro-4-ethoxyphenyl)-2-((6-fluorobenzo[d]oxazol-2-yl)amino)-6-methyl-1,4-dihydropyrimidine-5-carboxamido)isonicotinic acid was prepared according to Example 2 using methyl 2-(3-oxobutanamido)isonicotinate and 3-chloro-4-ethoxybenzaldehyde in Step 1, Example 2a using methyl 2-(4-(2-chloro-4-ethoxyphenyl)-2-((6-fluorobenzo[d]oxazol-2-yl)amino)-6-methyl-1,4-dihydropyrimidine-5-carboxamido)isonicotinate as starting material. MS m/z (M+H⁺) 565.2, ¹H NMR (400 MHz, DMSO-d₆) δ 13.59 (s, 1H), 10.60 (s, 1H), 10.10 (s, 1H), 10.09-10.05 (m, 1H), 8.43 (dd, J=5.1, 0.7 Hz, 1H), 8.42-8.37 (m, 1H), 7.46 (dd, J=5.1, 1.5 Hz, 1H), 7.44 (d, J=8.7 Hz, 1H), 7.37 (dd, J=8.6, 2.6 Hz, 1H), 7.31 (dd, J=8.6, 5.0 Hz, 1H), 7.01 (d, J=2.6 Hz, 1H), 7.00-6.96 (m, 1H), 6.88 (dd, J=8.7, 2.6 Hz, 1H), 6.07 (d, J=2.7 Hz, 1H), 3.97 (q, J=7.0 Hz, 2H), 2.21 (s, 3H), 1.23 (t, J=7.0 Hz, 3H).

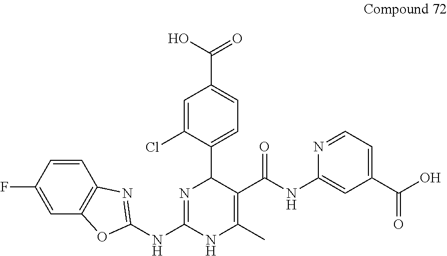 [SMILES: CC1=C(C(=O)Nc2cc(C(=O)O)ccn2)C(c2ccc(C(=O)O)cc2Cl)N=C(Nc2nc3ccc(F)cc3o2)N1]

The title compound 2-(4-(4-carboxy-2-chlorophenyl)-2-((6-fluorobenzo[d]oxazol-2-yl)amino)-6-methyl-1,4-dihydropyrimidine-5-carboxamido)isonicotinic acid was prepared according to Example 2 using methyl 2-(3-oxobutanamido)isonicotinate and methyl 3-chloro-4-formylbenzoate in Step 1, Example 2a using methyl 2-(4-(2-chloro-4-(methoxycarbonyl)phenyl)-2-((6-fluorobenzo[d]oxazol-2-yl)amino)-6-methyl-1,4-dihydropyrimidine-5-carboxamido)isonicotinate as starting material. MS m/z (M+H⁺) 564.9, ¹H NMR (400 MHz, DMSO-d₆) δ 13.54 (s, 1H), 13.32 (s, 1H), 10.68 (s, 1H), 10.22 (s, 1H), 10.15 (t, J=2.4 Hz, 1H), 8.44 (dd, J=5.1, 0.8 Hz, 1H), 8.39 (dd, J=1.4, 0.9 Hz, 1H), 7.90 (d, J=1.7 Hz, 1H), 7.87 (dd, J=8.0, 1.7 Hz, 1H), 7.66 (d, J=8.1 Hz, 1H), 7.47 (dd, J=5.1, 1.5 Hz, 1H), 7.38 (dd, J=8.5, 2.6 Hz, 1H), 7.33 (dd, J=8.6, 5.0 Hz, 1H), 7.00 (ddd, J=10.3, 8.7, 2.6 Hz, 1H), 6.15 (d, J=2.8 Hz, 1H), 2.23 (s, 3H).

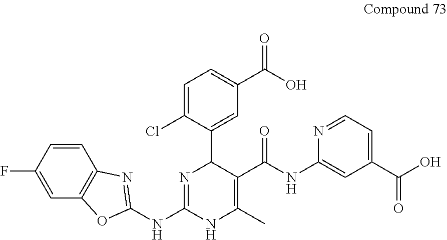 [SMILES: CC1=C(C(=O)Nc2cc(C(=O)O)ccn2)C(c2cc(C(=O)O)ccc2Cl)N=C(Nc2nc3ccc(F)cc3o2)N1]

The title compound 2-(4-(5-carboxy-2-chlorophenyl)-2-((6-fluorobenzo[d]oxazol-2-yl)amino)-6-methyl-1,4-dihydropyrimidine-5-carboxamido)isonicotinic acid was prepared according to Example 2 using methyl 2-(3-oxobutanamido)isonicotinate and methyl 4-chloro-3-formylbenzoate in Step 1, Example 2a using methyl 2-(4-(2-chloro-5-(methoxycarbonyl)phenyl)-2-((6-fluorobenzo[d]oxazol-2-yl)amino)-6-methyl-1,4-dihydropyrimidine-5-carboxamido)isonicotinate as starting material. MS m/z (M+H⁺) 565.2, ¹H NMR (400 MHz, DMSO-d₆) δ 13.34 (s, 2H), 10.71 (s, 1H), 10.23 (d, J=1.8 Hz, 1H), 10.06 (t, J=2.4 Hz, 1H), 8.43 (dd, J=5.1, 0.8 Hz, 1H), 8.38-8.36 (m, 1H), 7.98 (d, J=2.1 Hz, 1H), 7.81 (dd, J=8.3, 2.1 Hz, 1H), 7.58 (d, J=8.3 Hz, 1H), 7.46 (dd, J=5.1, 1.5 Hz, 1H), 7.38 (dd, J=8.5, 2.5 Hz, 1H), 7.33 (dd, J=8.6, 5.0 Hz, 1H), 6.99 (ddd, J=10.2, 8.7, 2.6 Hz, 1H), 6.19 (d, J=2.8 Hz, 1H), 2.21 (s, 3H).

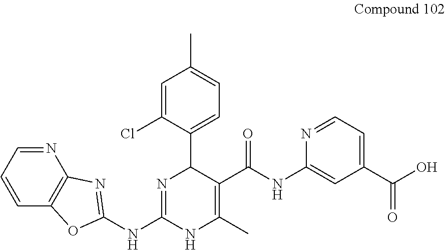 [SMILES: CC1=C(C(=O)Nc2cc(C(=O)O)ccn2)C(c2ccc(C)cc2Cl)N=C(Nc2nc3ncccc3o2)N1]

The title compound 2-(4-(2-chloro-4-methylphenyl)-6-methyl-2-(oxazolo[4,5-b]pyridin-2-ylamino)-1,4-dihydropyrimidine-5-carboxamido)isonicotinic acid was prepared according to Example 2 using methyl 2-(3-oxobutanamido)isonicotinate and 2-chloro-4-methylbenzaldehyde in Step 1, and using oxazolo[4,5-b]pyridin-2-amine in Step 2, Example 2a using methyl 2-(4-(2-chloro-4-methylphenyl)-6-methyl-2-(oxazolo[4,5-b]pyridin-2-ylamino)-1,4-dihydropyrimidine-5-carboxamido)isonicotinate as starting material. MS m/z (M+H⁺) 518.1, ¹H NMR (400 MHz, DMSO-d₆) δ 13.56 (s, 1H), 10.66 (s, 1H), 10.25 (s, 1H), 10.06 (t, J=2.3 Hz, 1H), 8.44 (dd, J=5.1, 0.7 Hz, 1H), 8.39 (s, 1H), 8.15 (dd, J=5.1, 1.4 Hz, 1H), 7.73 (dd, J=7.9, 1.4 Hz, 1H), 7.48-7.43 (m, 2H), 7.29 (s, 1H), 7.16 (d, J=7.3 Hz, 1H), 7.07 (dd, J=7.9, 5.1 Hz, 1H), 6.16 (d, J=2.6 Hz, 1H), 2.23 (s, 3H), 2.21 (s, 3H).

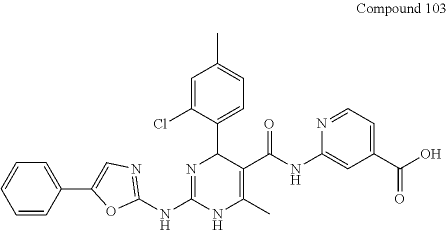 [SMILES: CC1=C(C(=O)Nc2cc(C(=O)O)ccn2)C(c2ccc(C)cc2Cl)N=C(Nc2ncc(-c3ccccc3)o2)N1]

The title compound 2-(4-(2-chloro-4-methylphenyl)-6-methyl-2-((5-phenyloxazol-2-yl)amino)-1,4-dihydropyrimidine-5-carboxamido)isonicotinic acid was prepared according to Example 2 using methyl 2-(3-oxobutanamido)isonicotinate and 2-chloro-4-methylbenzaldehyde in Step 1, and using 5-phenyloxazol-2-amine in Step 2, Example 2a using methyl 2-(4-(2-chloro-4-methylphenyl)-6-methyl-2-((5-phenyloxazol-2-yl)amino)-1,4-dihydropyrimidine-5-carboxamido)isonicotinate as starting material. MS m/z (M+H⁺) 543.1

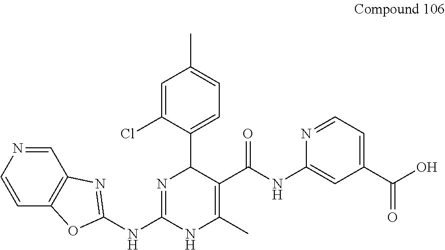 [SMILES: CC1=C(C(=O)Nc2cc(C(=O)O)ccn2)C(c2ccc(C)cc2Cl)N=C(Nc2nc3cnccc3o2)N1]

The title compound 2-(4-(2-chloro-4-methylphenyl)-6-methyl-2-(oxazolo[4,5-c]pyridin-2-ylamino)-1,4-dihydropyrimidine-5-carboxamido)isonicotinic acid was prepared according to Example 2 using methyl 2-(3-oxobutanamido)isonicotinate and 2-chloro-4-methylbenzaldehyde in Step 1, and using oxazolo[4,5-c]pyridin-2-amine in Step 2, Example 2a using methyl 2-(4-(2-chloro-4-methylphenyl)-6-methyl-2-((5-phenyloxazol-2-yl)amino)-1,4-dihydropyrimidine-5-carboxamido)isonicotinate as starting material. MS m/z (M+H⁺) 518.2, ¹H NMR (400 MHz, DMSO-d₆) δ 13.57 (s, 1H), 10.72 (s, 1H), 10.41 (s, 1H), 9.88 (s, 1H), 8.92 (s, 1H), 8.54 (d, J=6.1 Hz, 1H), 8.45 (d, J=5.1 Hz, 1H), 8.39 (s, 1H), 7.95 (d, J=6.1 Hz, 1H), 7.51-7.44 (m, 2H), 7.30 (s, 1H), 7.16 (d, J=8.3 Hz, 1H), 6.17 (d, J=2.9 Hz, 1H), 2.23 (s, 3H), 2.21 (s, 3H).

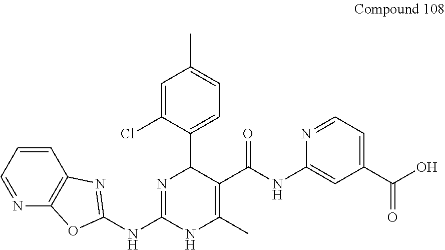 [SMILES: CC1=C(C(=O)Nc2cc(C(=O)O)ccn2)C(c2ccc(C)cc2Cl)N=C(Nc2nc3cccnc3o2)N1]

The title compound 2-(4-(2-chloro-4-methylphenyl)-6-methyl-2-(oxazolo[5,4-b]pyridin-2-ylamino)-1,4-dihydropyrimidine-5-carboxamido)isonicotinic acid was prepared according to Example 2 using methyl 2-(3-oxobutanamido)isonicotinate and 2-chloro-4-methylbenzaldehyde in Step 1, and using oxazolo[5,4-b]pyridin-2-amine in Step 2, Example 2a using methyl 2-(4-(2-chloro-4-methylphenyl)-6-methyl-2-(oxazolo[5,4-b]pyridin-2-ylamino)-1,4-dihydropyrimidine-5-carboxamido)isonicotinate as starting material. MS m/z (M+H⁺) 518.2, ¹H NMR (400 MHz, DMSO-d₆) δ 13.55 (s, 1H), 10.63 (s, 1H), 10.23 (s, 1H), 10.10 (s, 1H), 8.44 (d, J=5.1 Hz, 1H), 8.39 (s, 1H), 7.96 (dd, J=5.1, 1.4 Hz, 1H), 7.69 (dd, J=7.7, 1.4 Hz, 1H), 7.50-7.41 (m, 2H), 7.29 (s, 1H), 7.19 (dd, J=7.8, 5.0 Hz, 1H), 7.15 (d, J=7.8 Hz, 1H), 6.12 (d, J=2.1 Hz, 1H), 2.22 (s, 3H), 2.22 (s, 3H).

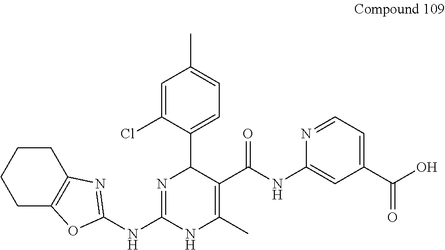 [SMILES: CC1=C(C(=O)Nc2cc(C(=O)O)ccn2)C(c2ccc(C)cc2Cl)N=C(Nc2nc3c(o2)CCCC3)N1]

The title compound 2-(4-(2-chloro-4-methylphenyl)-6-methyl-2-((4,5,6,7-tetrahydrobenzo[d]oxazol-2-yl)amino)-1,4-dihydropyrimidine-5-carboxamido)isonicotinic acid was prepared according to Example 2 using methyl 2-(3-oxobutanamido)isonicotinate and 2-chloro-4-methylbenzaldehyde in Step 1, and using 4,5,6,7-tetrahydrobenzo[d]oxazol-2-amine in Step 2, Example 2a using methyl 2-(4-(2-chloro-4-methylphenyl)-6-methyl-2-((4,5,6,7-tetrahydrobenzo[d]oxazol-2-yl)amino)-1,4-dihydropyrimidine-5-carboxamido)isonicotinate as starting material. MS m/z (M+H⁺) 521.2

### Example 2b

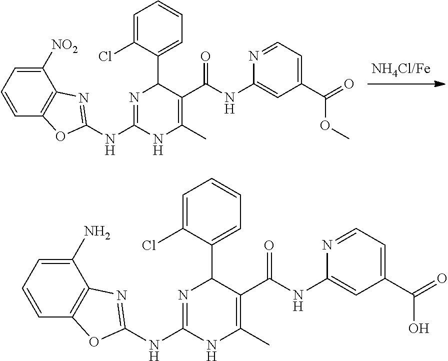 [SMILES: COC(=O)c1ccnc(NC(=O)C2=C(C)NC(Nc3nc4c([N+](=O)[O-])cccc4o3)=NC2c2ccccc2Cl)c1 ; CC1=C(C(=O)Nc2cc(C(=O)O)ccn2)C(c2ccccc2Cl)N=C(Nc2nc3c(N)cccc3o2)N1]

The mixture of methyl 2-(4-(2-chlorophenyl)-6-methyl-2-((7-nitrobenzo[d]oxazol-2-yl)amino)-1,4-dihydropyrimidine-5-carboxamido)isonicotinate (0.25 g, 0.445 mmol), ammonium chloride (0.048 g, 0.890 mmol) and iron (0.124 g, 2.224 mmol) in EtOH (2 mL) and water (2.000 mL) was stirred at 80° C. for 6 hr. After cooling, EtOAc was added and the reaction mixture was passed through Celite. The solvent was evaporated. The crude product was purified by ISCO (10-100% ACN/H₂O/0.1% TFA). ACN was removed by evaporation. NaHCO₃ was added to the residue until pH=8-10. The mixture was extracted with EtOAc. The organic layer was dried over MgSO₄ and concentrated. The pure product, (methyl 2-(2-((7-aminobenzo[d]oxazol-2-yl)amino)-4-(2-chlorophenyl)-6-methyl-1,4-dihydropyrimidine-5-carboxamido)isonicotinate), was chirally separated. (Intermediate to Compound 24)

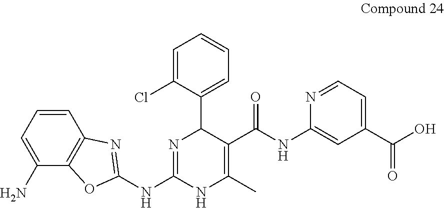 [SMILES: CC1=C(C(=O)Nc2cc(C(=O)O)ccn2)C(c2ccccc2Cl)N=C(Nc2nc3cccc(N)c3o2)N1]

The title compound (R)-2-(2-((7-aminobenzo[d]oxazol-2-yl)amino)-4-(2-chlorophenyl)-6-methyl-1,4-dihydropyrimidine-5-carboxamido)isonicotinic acid was prepared according to Example 2b using methyl 2-(4-(2-chlorophenyl)-6-methyl-2-((7-nitrobenzo[d]oxazol-2-yl)amino)-1,4-dihydropyrimidine-5-carboxamido)isonicotinate, Example 2a using (R)-methyl 2-(2-((7-aminobenzo[d]oxazol-2-yl)amino)-4-(2-chlorophenyl)-6-methyl-1,4-dihydropyrimidine-5-carboxamido)isonicotinate as starting material. MS m/z (M+H⁺) 518.2, ¹H NMR (400 MHz, DMSO-d₆) δ 13.56 (s, 1H), 10.68 (s, 1H), 10.16-10.11 (m, 2H), 8.44 (dd, J=5.1, 0.7 Hz, 1H), 8.42-8.36 (m, 1H), 7.56 (dd, J=7.6, 1.7 Hz, 1H), 7.50-7.42 (m, 2H), 7.36 (td, J=7.5, 1.4 Hz, 1H), 7.31 (td, J=7.7, 1.8 Hz, 1H), 6.94 (t, J=7.9 Hz, 1H), 6.71 (d, J=7.7 Hz, 1H), 6.55 (d, J=8.0 Hz, 1H), 6.13 (d, J=2.8 Hz, 1H), 3.81 (s, 2H), 2.22 (s, 3H).

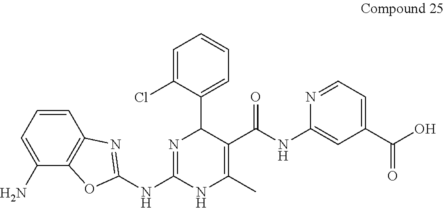 [SMILES: CC1=C(C(=O)Nc2cc(C(=O)O)ccn2)C(c2ccccc2Cl)N=C(Nc2nc3cccc(N)c3o2)N1]

The title compound (S)-2-(2-((7-aminobenzo[d]oxazol-2-yl)amino)-4-(2-chlorophenyl)-6-methyl-1,4-dihydropyrimidine-5-carboxamido)isonicotinic acid was prepared according to Example 2b using methyl 2-(4-(2-chlorophenyl)-6-methyl-2-((7-nitrobenzo[d]oxazol-2-yl)amino)-1,4-dihydropyrimidine-5-carboxamido)isonicotinate, Example 2a using (S)-methyl 2-(2-((7-aminobenzo[d]oxazol-2-yl)amino)-4-(2-chlorophenyl)-6-methyl-1,4-dihydropyrimidine-5-carboxamido)isonicotinate as starting material. MS m/z (M+H⁺) 518.2, ¹H NMR (400 MHz, DMSO-d₆) δ 13.57 (s, 1H), 10.65 (s, 1H), 10.19 (s, 1H), 10.07 (s, 1H), 8.44 (dd, J=5.1, 0.8 Hz, 1H), 8.39 (dd, J=1.5, 0.9 Hz, 1H), 7.56 (dd, J=7.6, 1.9 Hz, 1H), 7.47 (t, J=1.8 Hz, 1H), 7.45 (dd, J=4.8, 1.5 Hz, 1H), 7.36 (td, J=7.5, 1.5 Hz, 1H), 7.30 (td, J=7.5, 1.8 Hz, 1H), 6.90 (t, J=7.9 Hz, 1H), 6.65 (d, J=7.8 Hz, 1H), 6.49 (d, J=7.9 Hz, 1H), 6.13 (d, J=2.8 Hz, 1H), 3.61 (s, 2H), 2.23 (s, 3H).

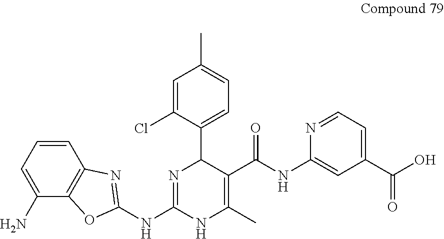 [SMILES: CC1=C(C(=O)Nc2cc(C(=O)O)ccn2)C(c2ccc(C)cc2Cl)N=C(Nc2nc3cccc(N)c3o2)N1]

The title compound 2-(2-((7-aminobenzo[d]oxazol-2-yl)amino)-4-(2-chloro-4-methylphenyl)-6-methyl-1,4-dihydropyrimidine-5-carboxamido)isonicotinic acid was prepared according to Example 2 using methyl 2-(3-oxobutanamido)isonicotinate and 2-chloro-4-methylbenzaldehyde in Step 1, and 7-nitrobenzo[d]oxazol-2-amine in Step 2, Example 2b using methyl 2-(4-(2-chloro-4-methylphenyl)-6-methyl-2-((7-nitrobenzo[d]oxazol-2-yl)amino)-1,4-dihydropyrimidine-5-carboxamido)isonicotinate, Example 2a using methyl 2-(2-((7-aminobenzo[d]oxazol-2-yl)amino)-4-(2-chloro-4-methylphenyl)-6-methyl-1,4-dihydropyrimidine-5-carboxamido)isonicotinate as starting material. MS m/z (M+H⁺) 532.0, ¹H NMR (400 MHz, DMSO-d₆) δ 13.58 (s, 1H), 10.61 (s, 1H), 10.20 (s, 1H), 9.98 (s, 1H), 8.44 (dd, J=5.1, 0.7 Hz, 1H), 8.40-8.38 (m, 1H), 7.46 (dd, J=5.1, 1.4 Hz, 1H), 7.44 (d, J=7.9 Hz, 1H), 7.31-7.25 (m, 1H), 7.14 (d, J=7.8 Hz, 1H), 6.86 (t, J=7.9 Hz, 1H), 6.59 (d, J=7.7 Hz, 1H), 6.42 (d, J=7.9 Hz, 1H), 6.08 (d, J=2.7 Hz, 1H), 3.49 (s, 5H), 2.22 (s, 3H).

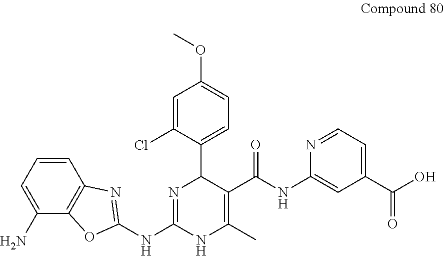 [SMILES: COc1ccc(C2N=C(Nc3nc4cccc(N)c4o3)NC(C)=C2C(=O)Nc2cc(C(=O)O)ccn2)c(Cl)c1]

The title compound 2-(2-((7-aminobenzo[d]oxazol-2-yl)amino)-4-(2-chloro-4-methoxyphenyl)-6-methyl-1,4-dihydropyrimidine-5-carboxamido)isonicotinic acid was prepared according to Example 2 using methyl 2-(3-oxobutanamido)isonicotinate and 2-chloro-4-methoxybenzaldehyde in Step 1, and 7-nitrobenzo[d]oxazol-2-amine in Step 2, Example 2b using methyl 2-(4-(2-chloro-4-methoxyphenyl)-6-methyl-2-((7-nitrobenzo[d]oxazol-2-yl)amino)-1,4-dihydropyrimidine-5-carboxamido)isonicotinate, Example 2a using methyl 2-(2-((7-aminobenzo[d]oxazol-2-yl)amino)-4-(2-chloro-4-methoxyphenyl)-6-methyl-1,4-dihydropyrimidine-5-carboxamido)isonicotinate as starting material. MS m/z (M+H⁺) 548.0, ¹H NMR (400 MHz, DMSO-d₆) δ 13.58 (s, 1H), 10.60 (s, 1H), 10.16 (s, 1H), 9.99 (s, 1H), 8.44 (dd, J=5.1, 0.7 Hz, 1H), 8.41-8.39 (m, 1H), 7.47 (dd, J=3.5, 1.5 Hz, 1H), 7.45 (d, J=5.6 Hz, 1H), 7.03 (d, J=2.6 Hz, 1H), 6.90 (dd, J=8.8, 2.8 Hz, 1H), 6.85 (d, J=7.9 Hz, 1H), 6.59 (d, J=7.8 Hz, 1H), 6.43 (d, J=8.0 Hz, 1H), 6.06 (d, J=2.6 Hz, 1H), 3.70 (s, 3H), 3.51 (s, 2H), 2.22 (s, 3H).

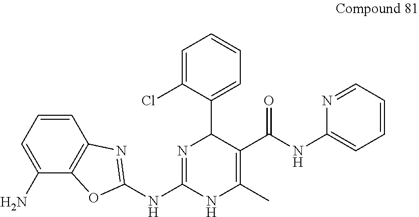 [SMILES: CC1=C(C(=O)Nc2ccccn2)C(c2ccccc2Cl)N=C(Nc2nc3cccc(N)c3o2)N1]

The title compound 2-((7-aminobenzo[d]oxazol-2-yl)amino)-4-(2-chlorophenyl)-6-methyl-N-(pyridin-2-yl)-1,4-dihydropyrimidine-5-carboxamide was prepared according to Example 2 using 3-oxo-N-(pyridin-2-yl)butanamide and 2-chloro-benzaldehyde in Step 1, and 7-nitrobenzo[d]oxazol-2-amine in Step 2, Example 2b using 4-(2-chlorophenyl)-6-methyl-2-((7-nitrobenzo[d]oxazol-2-yl)amino)-N-(pyridin-2-yl)-1,4-dihydropyrimidine-5-carboxamide as starting material. MS m/z (M+H⁺) 474.2, ¹H NMR (400 MHz, DMSO-d₆) δ 10.48 (d, J=9.9 Hz, 1H), 10.23-10.13 (m, 1H), 10.05 (s, 1H), 8.28 (dt, J=4.9, 2.6 Hz, 1H), 7.89 (dd, J=8.4, 5.5 Hz, 1H), 7.76-7.68 (m, 2H), 7.57 (dd, J=7.6, 1.9 Hz, 1H), 7.48-7.43 (m, 1H), 7.37-7.28 (m, 2H), 7.11-7.03 (m, 2H), 6.90 (t, J=7.9 Hz, 1H), 6.64 (d, J=7.7 Hz, 1H), 6.48 (d, J=8.0 Hz, 1H), 6.11 (d, J=2.8 Hz, 1H), 2.22 (s, 3H).

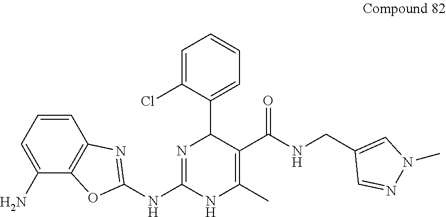 [SMILES: CC1=C(C(=O)NCc2cnn(C)c2)C(c2ccccc2Cl)N=C(Nc2nc3cccc(N)c3o2)N1]

The title compound 2-((7-aminobenzo[d]oxazol-2-yl)amino)-4-(2-chlorophenyl)-6-methyl-N-((1-methyl-1H-pyrazol-4-yl)methyl)-1,4-dihydropyrimidine-5-carboxamide was prepared according to Example 2 using N-((1-methyl-1H-pyrazol-4-yl)methyl)-3-oxobutanamide and 2-chloro-benzaldehyde in Step 1, and 7-nitrobenzo[d]oxazol-2-amine in Step 2, Example 2b using 4-(2-chlorophenyl)-6-methyl-N-((1-methyl-1H-pyrazol-4-yl)methyl)-2-((7-nitrobenzo[d]oxazol-2-yl)amino)-1,4-dihydropyrimidine-5-carboxamide as starting material. MS m/z (M+H⁺) 491.2, ¹H NMR (400 MHz, DMSO-d₆) δ 10.19 (s, 1H), 9.73 (s, 1H), 8.15 (t, J=5.7 Hz, 1H), 7.51-7.43 (m, 1H), 7.40 (dd, J=5.9, 3.6 Hz, 1H), 7.36-7.27 (m, 2H), 7.24 (s, 1H), 7.08 (s, 1H), 6.80 (t, J=7.9 Hz, 1H), 6.52 (dd, J=7.8, 1.0 Hz, 1H), 6.35 (dd, J=8.0, 1.0 Hz, 1H), 5.91 (d, J=2.8 Hz, 1H), 5.12 (s, 2H), 4.03 (dd, J=5.6, 2.0 Hz, 2H), 3.69 (s, 3H), 2.13 (s, 3H).

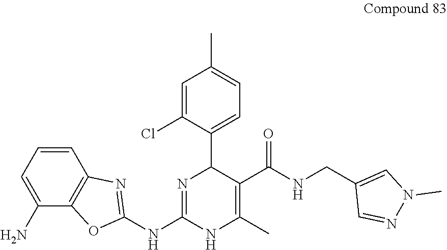 [SMILES: CC1=C(C(=O)NCc2cnn(C)c2)C(c2ccc(C)cc2Cl)N=C(Nc2nc3cccc(N)c3o2)N1]

The title compound 2-((7-aminobenzo[d]oxazol-2-yl)amino)-4-(2-chloro-4-methylphenyl)-6-methyl-N-((1-methyl-1H-pyrazol-4-yl)methyl)-1,4-dihydropyrimidine-5-carboxamide was prepared according to Example 2 using N-((1-methyl-1H-pyrazol-4-yl)methyl)-3-oxobutanamide and 2-chloro-4-methylbenzaldehyde in Step 1, and 7-nitrobenzo[d]oxazol-2-amine in Step 2, Example 2b using 4-(2-chloro-4-methylphenyl)-6-methyl-N-((1-methyl-1H-pyrazol-4-yl)methyl)-2-((7-nitrobenzo[d]oxazol-2-yl)amino)-1,4-dihydropyrimidine-5-carboxamide as starting material. MS m/z (M+H⁺) 505.2, ¹H NMR (400 MHz, DMSO-d₆) δ 10.16 (s, 1H), 9.68 (s, 1H), 8.13 (t, J=5.7 Hz, 1H), 7.32-7.22 (m, 3H), 7.11 (d, J=7.5 Hz, 2H), 6.80 (t, J=7.9 Hz, 1H), 6.51 (d, J=7.7 Hz, 1H), 6.35 (d, J=8.1 Hz, 1H), 5.87 (d, J=2.7 Hz, 1H), 5.11 (s, 2H), 4.03 (t, J=5.9 Hz, 2H), 3.69 (s, 3H), 2.24 (s, 3H), 2.12 (s, 3H).

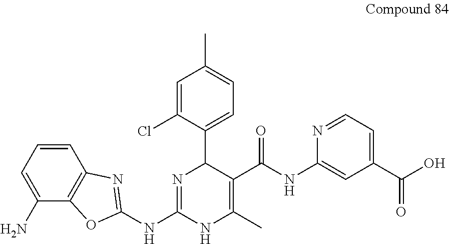 [SMILES: CC1=C(C(=O)Nc2cc(C(=O)O)ccn2)C(c2ccc(C)cc2Cl)N=C(Nc2nc3cccc(N)c3o2)N1]

The title compound (S)-2-(2-((7-aminobenzo[d]oxazol-2-yl)amino)-4-(2-chloro-4-methylphenyl)-6-methyl-1,4-dihydropyrimidine-5-carboxamido)isonicotinic acid was prepared according to Example 2 using methyl 2-(3-oxobutanamido)isonicotinate and 2-chloro-4-methylbenzaldehyde in Step 1, and 7-nitrobenzo[d]oxazol-2-amine in Step 2 as starting material, and following by the Chiral Separation. Example 2b using (S)-methyl 2-(4-(2-chloro-4-methylphenyl)-6-methyl-2-((7-nitrobenzo[d]oxazol-2-yl)amino)-1,4-dihydropyrimidine-5-carboxamido)isonicotinate, Example 2a using (S)-methyl 2-(2-((7-aminobenzo[d]oxazol-2-yl)amino)-4-(2-chloro-4-methylphenyl)-6-methyl-1,4-dihydropyrimidine-5-carboxamido)isonicotinate as starting material. MS m/z (M+H⁺) 532.2, ¹H NMR (400 MHz, DMSO-d₆) δ 13.56 (s, 1H), 10.69 (s, 1H), 10.15 (s, 1H), 10.05 (s, 1H), 8.44 (d, J=5.0 Hz, 1H), 8.39 (s, 1H), 7.47 (dd, J=5.0, 1.2 Hz, 1H), 7.44 (d, J=7.9 Hz, 1H), 7.28 (s, 1H), 7.16 (d, J=8.0 Hz, 1H), 7.00 (t, J=7.8 Hz, 1H), 6.79 (s, 1H), 6.66 (d, J=7.9 Hz, 1H), 6.13-6.07 (m, 1H), 4.25 (s, 2H), 2.23 (s, 3H), 2.20 (s, 3H).

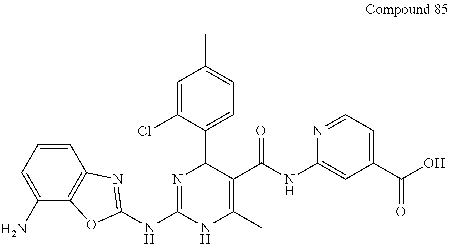 [SMILES: CC1=C(C(=O)Nc2cc(C(=O)O)ccn2)C(c2ccc(C)cc2Cl)N=C(Nc2nc3cccc(N)c3o2)N1]

The title compound (R)-2-(2-((7-aminobenzo[d]oxazol-2-yl)amino)-4-(2-chloro-4-methylphenyl)-6-methyl-1,4-dihydropyrimidine-5-carboxamido)isonicotinic acid was prepared according to Example 2 using methyl 2-(3-oxobutanamido)isonicotinate and 2-chloro-4-methylbenzaldehyde in Step 1, and 7-nitrobenzo[d]oxazol-2-amine in Step 2 as starting material, and following by the Chiral Separation. Example 2b using (R)-methyl 2-(4-(2-chloro-4-methylphenyl)-6-methyl-2-((7-nitrobenzo[d]oxazol-2-yl)amino)-1,4-dihydropyrimidine-5-carboxamido)isonicotinate, Example 2a using (R)-methyl 2-(2-((7-aminobenzo[d]oxazol-2-yl)amino)-4-(2-chloro-4-methylphenyl)-6-methyl-1,4-dihydropyrimidine-5-carboxamido)isonicotinate as starting material. MS m/z (M+H⁺) 532.2, ¹H NMR (400 MHz, DMSO-d₆) δ 13.54 (s, 1H), 10.69 (s, 1H), 10.17 (s, 1H), 10.03 (s, 1H), 8.44 (d, J=3.8 Hz, 1H), 8.38 (s, 1H), 7.47 (d, J=3.8 Hz, 1H), 7.44 (d, J=7.7 Hz, 1H), 7.27 (s, 1H), 7.15 (d, J=7.9 Hz, 1H), 7.01 (t, J=7.8 Hz, 1H), 6.83 (s, 1H), 6.70 (d, J=7.5 Hz, 1H), 6.10 (s, 1H), 4.56 (s, 2H), 2.22 (s, 3H), 2.19 (s, 3H).

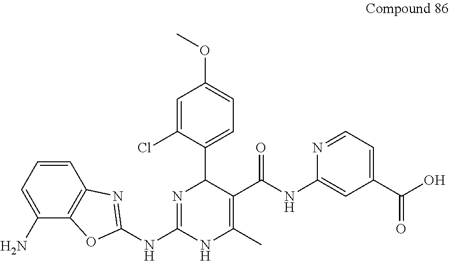 [SMILES: COc1ccc(C2N=C(Nc3nc4cccc(N)c4o3)NC(C)=C2C(=O)Nc2cc(C(=O)O)ccn2)c(Cl)c1]

The title compound (S)-2-(2-((7-aminobenzo[d]oxazol-2-yl)amino)-4-(2-chloro-4-methoxyphenyl)-6-methyl-1,4-dihydropyrimidine-5-carboxamido)isonicotinic acid was prepared according to Example 2 using methyl 2-(3-oxobutanamido)isonicotinate and 2-chloro-4-methoxybenzaldehyde in Step 1, and 7-nitrobenzo[d]oxazol-2-amine in Step 2 as starting material, and following by the Chiral Separation. Example 2b using (S)-methyl 2-(4-(2-chloro-4-methoxyphenyl)-6-methyl-2-((7-nitrobenzo[d]oxazol-2-yl)amino)-1,4-dihydropyrimidine-5-carboxamido)isonicotinate, Example 2a using (S)-methyl 2-(2-((7-aminobenzo[d]oxazol-2-yl)amino)-4-(2-chloro-4-methoxyphenyl)-6-methyl-1,4-dihydropyrimidine-5-carboxamido)isonicotinate as starting material. MS m/z (M+H⁺) 550.2, ¹H NMR (400 MHz, DMSO-d₆) δ 13.52 (s, 1H), 10.66 (s, 1H), 10.12 (s, 1H), 10.04 (s, 1H), 8.44 (d, J=5.0 Hz, 1H), 8.40 (s, 1H), 7.50-7.42 (m, 2H), 7.03 (d, J=2.5 Hz, 1H), 6.97 (t, J=7.9 Hz, 1H), 6.91 (dd, J=8.7, 2.5 Hz, 1H), 6.74 (d, J=7.8 Hz, 1H), 6.60 (d, J=8.1 Hz, 1H), 6.08 (d, J=2.7 Hz, 1H), 4.13 (s, 2H), 3.71 (s, 3H), 2.20 (s, 3H).

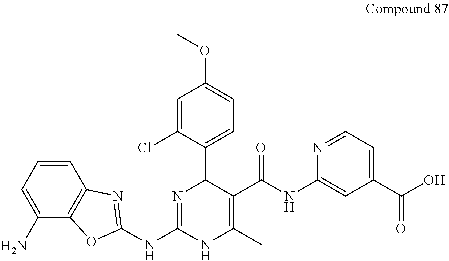 [SMILES: COc1ccc(C2N=C(Nc3nc4cccc(N)c4o3)NC(C)=C2C(=O)Nc2cc(C(=O)O)ccn2)c(Cl)c1]

The title compound (R)-2-(2-((7-aminobenzo[d]oxazol-2-yl)amino)-4-(2-chloro-4-methoxyphenyl)-6-methyl-1,4-dihydropyrimidine-5-carboxamido)isonicotinic acid was prepared according to Example 2 using methyl 2-(3-oxobutanamido)isonicotinate and 2-chloro-4-methoxybenzaldehyde in Step 1, and 7-nitrobenzo[d]oxazol-2-amine in Step 2 as starting material, and following by the Chiral Separation. Example 2b using (R)-methyl 2-(4-(2-chloro-4-methoxyphenyl)-6-methyl-2-((7-nitrobenzo[d]oxazol-2-yl)amino)-1,4-dihydropyrimidine-5-carboxamido)isonicotinate, Example 2a using (R)-methyl 2-(2-((7-aminobenzo[d]oxazol-2-yl)amino)-4-(2-chloro-4-methoxyphenyl)-6-methyl-1,4-dihydropyrimidine-5-carboxamido)isonicotinate as starting material. MS m/z (M+H⁺) 550.2, ¹H NMR (400 MHz, DMSO-d₆) δ 13.52 (s, 1H), 10.67 (s, 1H), 10.15 (s, 1H), 10.00 (s, 1H), 8.43 (s, 1H), 8.38 (s, 1H), 7.45 (d, J=8.4 Hz, 2H), 6.99 (d, J=12.3 Hz, 2H), 6.90 (d, J=8.3 Hz, 1H), 6.81 (s, 1H), 6.68 (s, 1H), 6.07 (s, 1H), 4.12 (s, 2H), 3.70 (s, 3H), 2.18 (s, 3H).

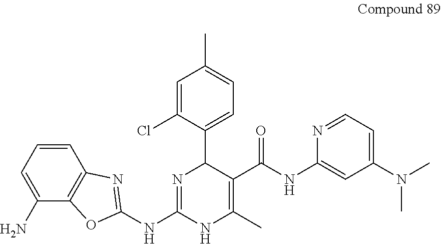 [SMILES: CC1=C(C(=O)Nc2cc(N(C)C)ccn2)C(c2ccc(C)cc2Cl)N=C(Nc2nc3cccc(N)c3o2)N1]

The title compound 2-((7-aminobenzo[d]oxazol-2-yl)amino)-4-(2-chloro-4-methylphenyl)-N-(4-(dimethylamino)pyridin-2-yl)-6-methyl-1,4-dihydropyrimidine-5-carboxamide was prepared according to Example 2 using N-(4-(dimethylamino)pyridin-2-yl)-3-oxobutanamide and 2-chloro-4-methylbenzaldehyde in Step 1, and 7-nitrobenzo[d]oxazol-2-amine in Step 2, Example 2b using 4-(2-chloro-4-methylphenyl)-N-(4-(dimethylamino)pyridin-2-yl)-6-methyl-2-((7-nitrobenzo[d]oxazol-2-yl)amino)-1,4-dihydropyrimidine-5-carboxamide as starting material. MS m/z (M+H⁺) 531.3, ¹H NMR (400 MHz, DMSO-d₆) δ 11.61 (s, 1H), 10.26 (d, J=10.7 Hz, 2H), 7.80 (d, J=6.8 Hz, 1H), 7.36 (d, J=7.6 Hz, 1H), 7.28 (s, 1H), 7.13 (d, J=7.9 Hz, 1H), 6.90 (t, J=6.6 Hz, 1H), 6.79 (s, 1H), 6.71 (dd, J=22.2, 7.1 Hz, 2H), 6.52 (d, J=7.7 Hz, 1H), 6.11 (s, 1H), 3.53 (s, 2H), 3.09 (s, 6H), 2.31 (s, 3H), 2.22 (s, 3H).

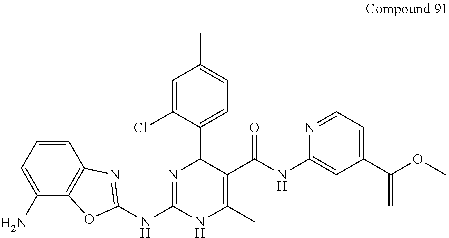 [SMILES: C=C(OC)c1ccnc(NC(=O)C2=C(C)NC(Nc3nc4cccc(N)c4o3)=NC2c2ccc(C)cc2Cl)c1]

The title compound methyl 2-(2-((7-aminobenzo[d]oxazol-2-yl)amino)-4-(2-chloro-4-methylphenyl)-6-methyl-1,4-dihydropyrimidine-5-carboxamido)isonicotinate was prepared according to Example 2 using methyl 2-(3-oxobutanamido)isonicotinate and 2-chloro-4-methylbenzaldehyde in Step 1, and 7-nitrobenzo[d]oxazol-2-amine in Step 2, Example 2b using methyl 2-(4-(2-chloro-4-methylphenyl)-6-methyl-2-((7-nitrobenzo[d]oxazol-2-yl)amino)-1,4-dihydropyrimidine-5-carboxamido)isonicotinate as starting material. MS m/z (M+H⁺) 546.3, ¹H NMR (400 MHz, DMSO-d₆) δ 10.61 (s, 1H), 10.25 (s, 1H), 9.93 (s, 1H), 8.47 (d, J=5.1 Hz, 1H), 8.42 (d, J=1.2 Hz, 1H), 7.48 (dd, J=5.1, 1.5 Hz, 1H), 7.43 (d, J=7.9 Hz, 1H), 7.28 (d, J=1.7 Hz, 1H), 7.14 (dd, J=8.1, 1.7 Hz, 1H), 6.82 (t, J=7.9 Hz, 1H), 6.54 (d, J=7.7 Hz, 1H), 6.37 (d, J=7.9 Hz, 1H), 6.08 (d, J=2.8 Hz, 1H), 5.13 (s, 2H), 3.84 (s, 3H), 2.24 (s, 3H), 2.22 (s, 3H).

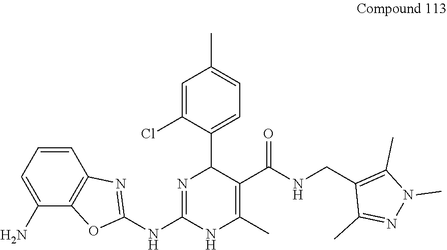 [SMILES: CC1=C(C(=O)NCc2c(C)nn(C)c2C)C(c2ccc(C)cc2Cl)N=C(Nc2nc3cccc(N)c3o2)N1]

The title compound 2-((7-aminobenzo[d]oxazol-2-yl)amino)-4-(2-chloro-4-methylphenyl)-6-methyl-N-((1,3,5-trimethyl-1H-pyrazol-4-yl)methyl)-1,4-dihydropyrimidine-5-carboxamide was prepared according to Example 2 using 3-oxo-N-((1,3,5-trimethyl-1H-pyrazol-4-yl)methyl)butanamide and 2-chloro-4-methylbenzaldehyde in Step 1, and 7-nitrobenzo[d]oxazol-2-amine in Step 2, Example 2b using 4-(2-chloro-4-methylphenyl)-6-methyl-2-((7-nitrobenzo[d]oxazol-2-yl)amino)-N-((1,3,5-trimethyl-1H-pyrazol-4-yl)methyl)-1,4-dihydropyrimidine-5-carboxamide as starting material. MS m/z (M+H⁺) 533.2, ¹H NMR (400 MHz, DMSO-d₆) δ 10.00 (s, 1H), 9.80 (s, 1H), 7.99 (t, J=5.3 Hz, 1H), 7.25 (d, J=7.8 Hz, 2H), 7.09 (d, J=8.2 Hz, 1H), 6.88 (t, J=7.9 Hz, 1H), 6.59 (d, J=7.8 Hz, 1H), 6.46 (d, J=8.0 Hz, 1H), 5.85 (t, J=1.8 Hz, 1H), 3.99-3.83 (m, 2H), 3.54 (s, 3H), 2.23 (s, 3H), 2.04 (s, 3H), 2.03 (d, J=1.2 Hz, 2H), 2.01 (s, 3H), 1.90 (s, 3H).

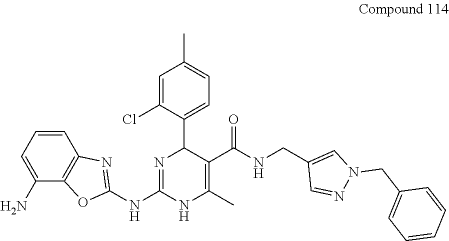 [SMILES: CC1=C(C(=O)NCc2cnn(Cc3ccccc3)c2)C(c2ccc(C)cc2Cl)N=C(Nc2nc3cccc(N)c3o2)N1]

The title compound 2-((7-aminobenzo[d]oxazol-2-yl)amino)-N-((1-benzyl-1H-pyrazol-4-yl)methyl)-4-(2-chloro-4-methylphenyl)-6-methyl-1,4-dihydropyrimidine-5-carboxamide was prepared according to Example 2 using N-((1-benzyl-1H-pyrazol-4-yl)methyl)-3-oxobutanamide and 2-chloro-4-methylbenzaldehyde in Step 1, and 7-nitrobenzo[d]oxazol-2-amine in Step 2, Example 2b using N-((1-benzyl-1H-pyrazol-4-yl)methyl)-4-(2-chloro-4-methylphenyl)-6-methyl-2-((7-nitrobenzo[d]oxazol-2-yl)amino)-1,4-dihydropyrimidine-5-carboxamide as starting material. MS m/z (M+H⁺) 581.2, ¹H NMR (400 MHz, DMSO-d₆) δ 10.11 (s, 1H), 9.73 (s, 1H), 8.21-8.13 (m, 1H), 7.40 (s, 1H), 7.34-7.20 (m, 5H), 7.19-7.06 (m, 4H), 6.83 (t, J=7.8 Hz, 1H), 6.55 (d, J=7.8 Hz, 1H), 6.40 (d, J=8.0 Hz, 1H), 5.87 (s, 1H), 5.18 (s, 2H), 4.13-3.97 (m, 2H), 2.22 (s, 3H), 2.10 (s, 3H), 2.04 (s, 2H).

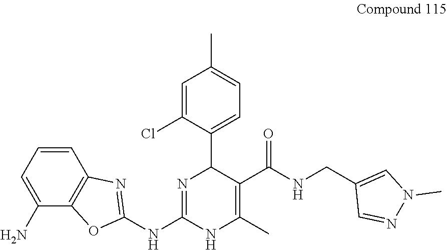 [SMILES: CC1=C(C(=O)NCc2cnn(C)c2)C(c2ccc(C)cc2Cl)N=C(Nc2nc3cccc(N)c3o2)N1]

The title compound (R)-2-((7-aminobenzo[d]oxazol-2-yl)amino)-4-(2-chloro-4-methylphenyl)-6-methyl-N-((1-methyl-1H-pyrazol-4-yl)methyl)-1,4-dihydropyrimidine-5-carboxamide was prepared according to Example 2 using N-((1-methyl-1H-pyrazol-4-yl)methyl)-3-oxobutanamide and 2-chloro-4-methylbenzaldehyde in Step 1, and 7-nitrobenzo[d]oxazol-2-amine in Step 2, Example 2b using 4-(2-chloro-4-methylphenyl)-6-methyl-N-((1-methyl-1H-pyrazol-4-yl)methyl)-2-((7-nitrobenzo[d]oxazol-2-yl)amino)-1,4-dihydropyrimidine-5-carboxamide as starting material, followed by Chiral Separation. MS m/z (M+H⁺) 505.2, ¹H NMR (400 MHz, DMSO-d₆) δ 10.15 (s, 1H), 9.68 (s, 1H), 8.13 (t, J=5.7 Hz, 1H), 7.32-7.22 (m, 3H), 7.11 (d, J=6.3 Hz, 2H), 6.80 (t, J=7.9 Hz, 1H), 6.51 (dd, J=7.8, 1.1 Hz, 1H), 6.35 (dd, J=8.0, 1.1 Hz, 1H), 5.87 (t, J=1.9 Hz, 1H), 5.10 (s, 2H), 4.11-3.95 (m, 2H), 3.69 (s, 3H), 2.24 (s, 3H), 2.12 (s, 3H).

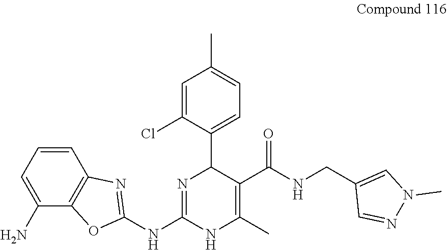 [SMILES: CC1=C(C(=O)NCc2cnn(C)c2)C(c2ccc(C)cc2Cl)N=C(Nc2nc3cccc(N)c3o2)N1]

The title compound (S)-2-((7-aminobenzo[d]oxazol-2-yl)amino)-4-(2-chloro-4-methylphenyl)-6-methyl-N-((1-methyl-1H-pyrazol-4-yl)methyl)-1,4-dihydropyrimidine-5-carboxamide was prepared according to Example 2 using N-((1-methyl-1H-pyrazol-4-yl)methyl)-3-oxobutanamide and 2-chloro-4-methylbenzaldehyde in Step 1, and 7-nitrobenzo[d]oxazol-2-amine in Step 2, Example 2b using 4-(2-chloro-4-methylphenyl)-6-methyl-N-((1-methyl-1H-pyrazol-4-yl)methyl)-2-((7-nitrobenzo[d]oxazol-2-yl)amino)-1,4-dihydropyrimidine-5-carboxamide as starting material, followed by Chiral Separation. MS m/z (M+H⁺) 505.2, ¹H NMR (400 MHz, DMSO-d₆) δ 10.15 (s, 1H), 9.68 (s, 1H), 8.14 (t, J=5.7 Hz, 1H), 7.31-7.22 (m, 3H), 7.15-7.07 (m, 2H), 6.80 (t, J=7.8 Hz, 1H), 6.51 (dd, J=7.8, 1.0 Hz, 1H), 6.35 (dd, J=8.0, 1.1 Hz, 1H), 5.90-5.84 (m, 1H), 5.10 (s, 2H), 4.11-3.95 (m, 2H), 3.69 (s, 3H), 2.24 (s, 3H), 2.12 (s, 3H).

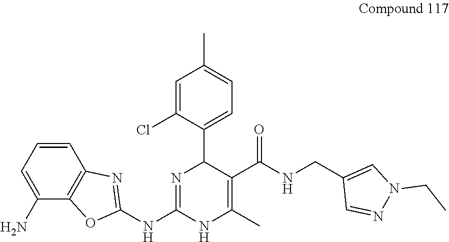 [SMILES: CCn1cc(CNC(=O)C2=C(C)NC(Nc3nc4cccc(N)c4o3)=NC2c2ccc(C)cc2Cl)cn1]

The title compound 2-((7-aminobenzo[d]oxazol-2-yl)amino)-4-(2-chloro-4-methylphenyl)-6-methyl-N-((1-ethyl-1H-pyrazol-4-yl)methyl)-1,4-dihydropyrimidine-5-carboxamide was prepared according to Example 2 using N-((1-ethyl-1H-pyrazol-4-yl)methyl)-3-oxobutanamide and 2-chloro-4-methylbenzaldehyde in Step 1, and 7-nitrobenzo[d]oxazol-2-amine in Step 2, Example 2b using 4-(2-chloro-4-methylphenyl)-6-methyl-N-((1-ethyl-1H-pyrazol-4-yl)methyl)-2-((7-nitrobenzo[d]oxazol-2-yl)amino)-1,4-dihydropyrimidine-5-carboxamide as starting material. MS m/z (M+H⁺) 519.2, ¹H NMR (400 MHz, DMSO-d₆) δ 10.19-10.14 (m, 1H), 9.68 (s, 1H), 8.15 (t, J=5.7 Hz, 1H), 7.32-7.23 (m, 3H), 7.11 (d, J=6.3 Hz, 2H), 6.80 (t, J=7.9 Hz, 1H), 6.51 (dd, J=7.8, 1.1 Hz, 1H), 6.35 (dd, J=8.0, 1.1 Hz, 1H), 5.91-5.85 (m, 1H), 5.11 (s, 2H), 4.04 (t, J=5.3 Hz, 2H), 3.97 (q, J=7.2 Hz, 2H), 2.24 (s, 3H), 2.12 (s, 3H), 1.25 (t, J=7.3 Hz, 3H).

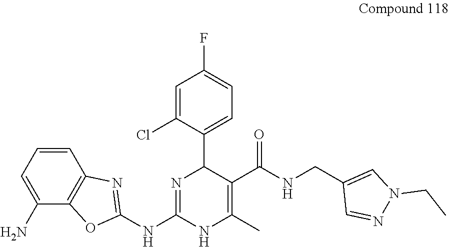 [SMILES: CCn1cc(CNC(=O)C2=C(C)NC(Nc3nc4cccc(N)c4o3)=NC2c2ccc(F)cc2Cl)cn1]

The title compound 2-((7-aminobenzo[d]oxazol-2-yl)amino)-4-(2-chloro-4-fluorophenyl)-N-((1-ethyl-1H-pyrazol-4-yl)methyl)-6-methyl-1,4-dihydropyrimidine-5-carboxamide was prepared according to Example 2 using N-((1-ethyl-1H-pyrazol-4-yl)methyl)-3-oxobutanamide and 2-chloro-4-fluorobenzaldehyde in Step 1, and 7-nitrobenzo[d]oxazol-2-amine in Step 2, Example 2b using 4-(2-chloro-4-fluorophenyl)-N-((1-ethyl-1H-pyrazol-4-yl)methyl)-6-methyl-2-((7-nitrobenzo[d]oxazol-2-yl)amino)-1,4-dihydropyrimidine-5-carboxamide as starting material. MS m/z (M+H⁺) 523.2, ¹H NMR (400 MHz, DMSO-d₆) δ 10.05 (s, 1H), 9.89-9.84 (m, 1H), 8.18 (t, J=5.8 Hz, 1H), 7.45 (ddd, J=11.1, 8.1, 4.3 Hz, 2H), 7.30 (s, 1H), 7.20 (td, J=8.5, 2.6 Hz, 1H), 7.10 (s, 1H), 6.87 (t, J=7.9 Hz, 1H), 6.59 (d, J=7.8 Hz, 1H), 6.45 (d, J=8.0 Hz, 1H), 5.93-5.87 (m, 1H), 4.04 (d, J=5.6 Hz, 2H), 3.98 (q, J=7.3 Hz, 2H), 2.11 (s, 3H), 1.26 (t, J=7.3 Hz, 3H). NH₂ did not shown in ¹H-NMR.

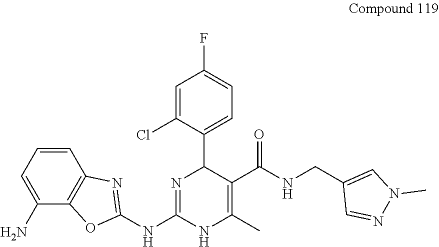 [SMILES: CC1=C(C(=O)NCc2cnn(C)c2)C(c2ccc(F)cc2Cl)N=C(Nc2nc3cccc(N)c3o2)N1]

The title compound 2-((7-aminobenzo[d]oxazol-2-yl)amino)-4-(2-chloro-4-fluorophenyl)-N-((1-methyl-1H-pyrazol-4-yl)methyl)-6-methyl-1,4-dihydropyrimidine-5-carboxamide was prepared according to Example 2 using N-((1-methyl-1H-pyrazol-4-yl)methyl)-3-oxobutanamide and 2-chloro-4-fluorobenzaldehyde in Step 1, and 7-nitrobenzo[d]oxazol-2-amine in Step 2, Example 2b using 4-(2-chloro-4-fluorophenyl)-N-((1-methyl-1H-pyrazol-4-yl)methyl)-6-methyl-2-((7-nitrobenzo[d]oxazol-2-yl)amino)-1,4-dihydropyrimidine-5-carboxamide as starting material. MS m/z (M+H⁺) 509.2, ¹H NMR (400 MHz, DMSO-d₆) δ 10.05 (s, 1H), 9.86 (s, 1H), 8.17 (t, J=5.8 Hz, 1H), 7.50-7.39 (m, 2H), 7.27 (s, 1H), 7.20 (td, J=8.5, 2.6 Hz, 1H), 7.09 (s, 1H), 6.87 (t, J=7.9 Hz, 1H), 6.59 (d, J=7.8 Hz, 1H), 6.44 (d, J=8.0 Hz, 1H), 5.89 (d, J=2.7 Hz, 1H), 4.03 (d, J=5.6 Hz, 2H), 3.70 (s, 3H), 2.11 (s, 3H). NH₂ did not shown in ¹H-NMR.

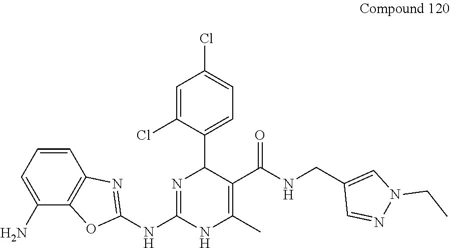 [SMILES: CCn1cc(CNC(=O)C2=C(C)NC(Nc3nc4cccc(N)c4o3)=NC2c2ccc(Cl)cc2Cl)cn1]

The title compound 2-((7-aminobenzo[d]oxazol-2-yl)amino)-4-(2,4-dichlorophenyl)-N-((1-ethyl-1H-pyrazol-4-yl)methyl)-6-methyl-1,4-dihydropyrimidine-5-carboxamide was prepared according to Example 2 using N-((1-ethyl-1H-pyrazol-4-yl)methyl)-3-oxobutanamide and 2,4-dichlorobenzaldehyde in Step 1, and 7-nitrobenzo[d]oxazol-2-amine in Step 2, Example 2b using 4-(2,4-dichlorophenyl)-N-((1-ethyl-1H-pyrazol-4-yl)methyl)-6-methyl-2-((7-nitrobenzo[d]oxazol-2-yl)amino)-1,4-dihydropyrimidine-5-carboxamide as starting material. MS m/z (M+H⁺) 539.2

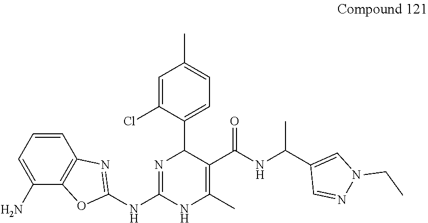 [SMILES: CCn1cc(C(C)NC(=O)C2=C(C)NC(Nc3nc4cccc(N)c4o3)=NC2c2ccc(C)cc2Cl)cn1]

The title compound 2-((7-aminobenzo[d]oxazol-2-yl)amino)-4-(2-chloro-4-methylphenyl)-6-methyl-N-((1-ethyl-1H-pyrazol-4-yl)methyl)-1,4-dihydropyrimidine-5-carboxamide was prepared according to Example 2 using N-(1-(1-ethyl-1H-pyrazol-4-yl)ethyl)-3-oxobutanamide and 2-chloro-4-methylbenzaldehyde in Step 1, and 7-nitrobenzo[d]oxazol-2-amine in Step 2, Example 2b using 4-(2-chloro-4-methylphenyl)-N-(1-(1-ethyl-1H-pyrazol-4-yl)ethyl)-6-methyl-2-((7-nitrobenzo[d]oxazol-2-yl)amino)-1,4-dihydropyrimidine-5-carboxamide as starting material. MS m/z (M+H⁺) 533.2, ¹H NMR (400 MHz, DMSO-d₆) δ 10.12 (s, 1H), 9.74 (s, 1H), 8.03 (d, J=8.2 Hz, 1H), 7.31 (d, J=8.3 Hz, 2H), 7.27 (s, 1H), 7.14 (d, J=5.1 Hz, 2H), 6.85 (t, J=7.9 Hz, 1H), 6.57 (d, J=7.8 Hz, 1H), 6.42 (d, J=8.0 Hz, 1H), 5.92 (d, J=2.8 Hz, 1H), 4.83 (p, J=7.0 Hz, 1H), 3.97 (q, J=7.3 Hz, 2H), 2.25 (s, 3H), 2.11 (s, 3H), 1.26 (t, J=7.3 Hz, 3H), 1.21 (d, J=6.8 Hz, 3H). NH₂ did not shown in ¹H-NMR.

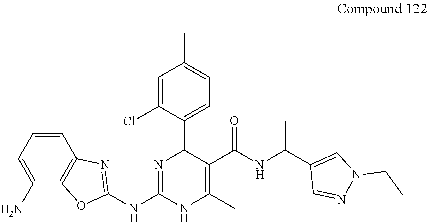 [SMILES: CCn1cc(C(C)NC(=O)C2=C(C)NC(Nc3nc4cccc(N)c4o3)=NC2c2ccc(C)cc2Cl)cn1]

The title compound 2-((7-aminobenzo[d]oxazol-2-yl)amino)-4-(2-chloro-4-methylphenyl)-6-methyl-N-((1-ethyl-1H-pyrazol-4-yl)methyl)-1,4-dihydropyrimidine-5-carboxamide was prepared according to Example 2 using N-(1-(1-ethyl-1H-pyrazol-4-yl)ethyl)-3-oxobutanamide and 2-chloro-4-methylbenzaldehyde in Step 1, and 7-nitrobenzo[d]oxazol-2-amine in Step 2, Example 2b using 4-(2-chloro-4-methylphenyl)-N-(1-(1-ethyl-1H-pyrazol-4-yl)ethyl)-6-methyl-2-((7-nitrobenzo[d]oxazol-2-yl)amino)-1,4-dihydropyrimidine-5-carboxamide as starting material. MS m/z (M+H⁺) 533.2, ¹H NMR (400 MHz, DMSO-d₆) δ 10.00 (s, 1H), 9.75 (s, 1H), 8.07 (d, J=8.4 Hz, 1H), 7.35 (d, J=7.9 Hz, 1H), 7.30 (s, 1H), 7.17 (s, 1H), 7.14 (d, J=8.0 Hz, 1H), 7.05 (s, 1H), 6.85 (t, J=7.9 Hz, 1H), 6.55 (d, J=7.8 Hz, 1H), 6.41 (d, J=8.0 Hz, 1H), 5.91 (s, 1H), 4.83 (q, J=7.3 Hz, 1H), 3.95 (q, J=7.3 Hz, 2H), 2.26 (s, 3H), 2.10 (s, 3H), 1.25 (q, J=7.0 Hz, 6H). NH₂ did not shown in ¹H-NMR.

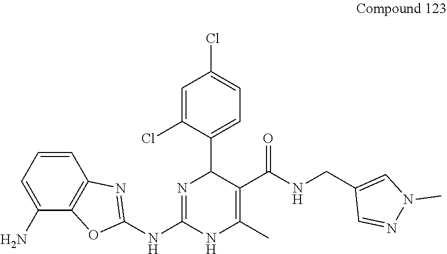 [SMILES: CC1=C(C(=O)NCc2cnn(C)c2)C(c2ccc(Cl)cc2Cl)N=C(Nc2nc3cccc(N)c3o2)N1]

The title compound 2-((7-aminobenzo[d]oxazol-2-yl)amino)-4-(2,4-dichlorophenyl)-N-((1-methyl-1H-pyrazol-4-yl)methyl)-6-methyl-1,4-dihydropyrimidine-5-carboxamide was prepared according to Example 2 using N-((1-methyl-1H-pyrazol-4-yl)methyl)-3-oxobutanamide and 2,4-dichlorobenzaldehyde in Step 1, and 7-nitrobenzo[d]oxazol-2-amine in Step 2, Example 2b using 4-(2,4-dichlorophenyl)-N-((1-methyl-1H-pyrazol-4-yl)methyl)-6-methyl-2-((7-nitrobenzo[d]oxazol-2-yl)amino)-1,4-dihydropyrimidine-5-carboxamide as starting material. MS m/z (M+H⁺) 525.1

### Example 2c

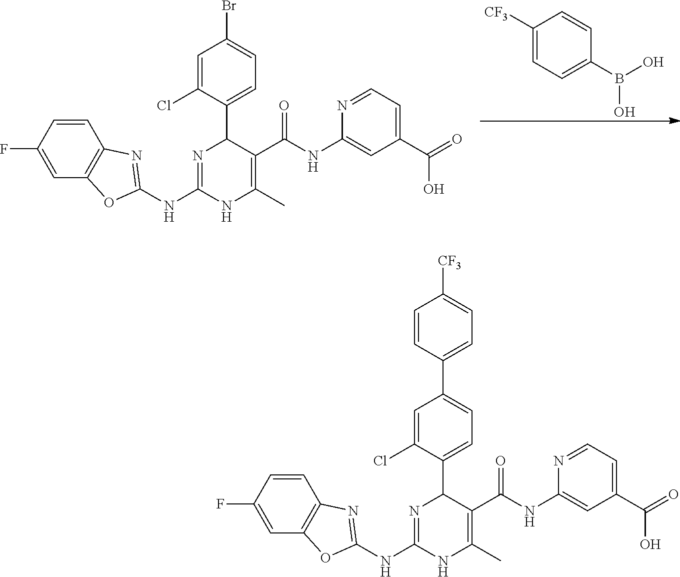 [SMILES: CC1=C(C(=O)Nc2cc(C(=O)O)ccn2)C(c2ccc(Br)cc2Cl)N=C(Nc2nc3ccc(F)cc3o2)N1 ; OB(O)c1ccc(C(F)(F)F)cc1 ; CC1=C(C(=O)Nc2cc(C(=O)O)ccn2)C(c2ccc(-c3ccc(C(F)(F)F)cc3)cc2Cl)N=C(Nc2nc3ccc(F)cc3o2)N1]

To a mixture of (4-(trifluoromethyl)phenyl)boronic acid (20.14 mg, 0.106 mmol), potassium carbonate (44.0 mg, 0.318 mmol) and PdCl₂(dppf)-CH₂Cl₂ (4.33 mg, 5.30 μmol) was added a solution of 2-(4-(4-bromo-2-chlorophenyl)-2-((6-fluorobenzo[d]oxazol-2-yl)amino)-6-methyl-1,4-dihydropyrimidine-5-carboxamido)isonicotinic acid (31.8 mg, 0.053 mmol) in Dioxane (0.6 mL) and Water (0.200 mL). The mixture was stirred at 80° C. for 3 hrs. After cooled to R.T., EtOAc (3 mL) and 1 N HCl (3 mL) were added to the mixture. The organic layer was separated, and washed with brine, dried over MgSO₄, and concentrated. The crude product was purified by ISCO (10-100%, ACN/H₂O, 0.01% TFA). MS m/z (M+H⁺) 665.2, ¹H NMR (400 MHz, DMSO-d₆) δ 13.58 (s, 1H), 10.71 (s, 1H), 10.21 (s, 1H), 8.45 (dd, J=5.1, 0.8 Hz, 1H), 8.42 (dd, J=1.4, 0.9 Hz, 1H), 7.91-7.82 (m, 3H), 7.71 (ddd, J=22.4, 20.8, 8.2 Hz, 4H), 7.47 (dd, J=5.1, 1.5 Hz, 2H), 7.38 (dd, J=8.6, 2.7 Hz, 1H), 7.34 (dd, J=8.6, 5.0 Hz, 1H), 7.04-6.97 (m, 1H), 6.17 (d, J=2.6 Hz, 1H), 2.26 (s, 3H). (Compound 68)

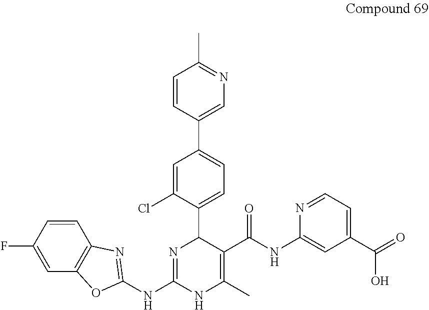 [SMILES: CC1=C(C(=O)Nc2cc(C(=O)O)ccn2)C(c2ccc(-c3ccc(C)nc3)cc2Cl)N=C(Nc2nc3ccc(F)cc3o2)N1]

The title compound 2-(4-(2-chloro-4-(6-methylpyridin-3-yl)phenyl)-2-((6-fluorobenzo[d]oxazol-2-yl)amino)-6-methyl-1,4-dihydropyrimidine-5-carboxamido)isonicotinic acid was prepared according to Example 2c using (6-methylpyridin-3-yl)boronic acid as starting material. MS m/z (M+H⁺) 612.2, ¹H NMR (400 MHz, DMSO-d₆) δ 13.60 (s, 1H), 10.72 (s, 1H), 10.23 (s, 2H), 8.94 (d, J=2.3 Hz, 1H), 8.45 (dd, J=5.1, 0.8 Hz, 1H), 8.42 (dd, J=1.5, 0.8 Hz, 2H), 7.93 (d, J=1.9 Hz, 1H), 7.75 (dd, J=8.1, 1.9 Hz, 1H), 7.66 (dd, J=8.3, 4.1 Hz, 2H), 7.49-7.44 (m, 1H), 7.39 (dd, J=8.6, 2.5 Hz, 1H), 7.33 (dd, J=8.6, 5.0 Hz, 1H), 7.01 (ddd, J=10.1, 8.6, 2.5 Hz, 1H), 6.18 (d, J=2.7 Hz, 1H), 2.58 (s, 3H), 2.26 (s, 3H).

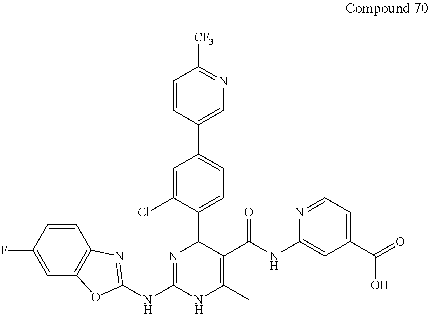 [SMILES: CC1=C(C(=O)Nc2cc(C(=O)O)ccn2)C(c2ccc(-c3ccc(C(F)(F)F)nc3)cc2Cl)N=C(Nc2nc3ccc(F)cc3o2)N1]

The title compound 2-(4-(2-chloro-4-(6-(trifluoromethyl)pyridin-3-yl)phenyl)-2-((6-fluorobenzo[d]oxazol-2-yl)amino)-6-methyl-1,4-dihydropyrimidine-5-carboxamido)isonicotinic acid was prepared according to Example 2c using (6-(trifluoromethyl)pyridin-3-yl)boronic acid as starting material. MS m/z (M+H⁺) 66.2, ¹H NMR (400 MHz, DMSO-d₆) δ 13.60 (s, 1H), 10.73 (s, 1H), 10.23 (s, 2H), 9.06 (d, J=2.2 Hz, 1H), 8.45 (dd, J=5.1, 0.8 Hz, 1H), 8.42 (dd, J=1.4, 0.9 Hz, 1H), 8.36 (dd, J=8.2, 2.3 Hz, 1H), 7.97 (d, J=1.9 Hz, 1H), 7.94 (d, J=8.2 Hz, 1H), 7.79 (dd, J=8.1, 1.9 Hz, 1H), 7.69 (d, J=8.1 Hz, 1H), 7.47 (dd, J=5.6, 1.2 Hz, 1H), 7.39 (dd, J=8.6, 2.5 Hz, 1H), 7.34 (dd, J=8.6, 5.0 Hz, 1H), 7.00 (ddd, J=10.3, 8.6, 2.5 Hz, 1H), 6.18 (d, J=2.6 Hz, 1H), 2.26 (s, 3H).

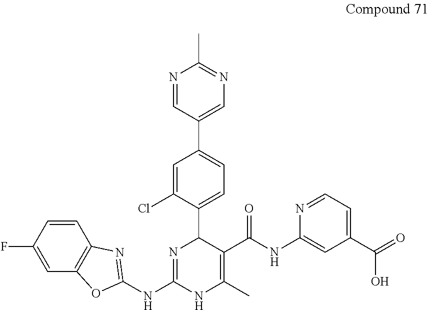 [SMILES: CC1=C(C(=O)Nc2cc(C(=O)O)ccn2)C(c2ccc(-c3cnc(C)nc3)cc2Cl)N=C(Nc2nc3ccc(F)cc3o2)N1]

The title compound 2-(4-(2-chloro-4-(2-methylpyrimidin-5-yl)phenyl)-2-((6-fluorobenzo[d]oxazol-2-yl)amino)-6-methyl-1,4-dihydropyrimidine-5-carboxamido)isonicotinic acid was prepared according to Example 2c using (2-methylpyrimidin-5-yl)boronic acid as starting material. MS m/z (M+H⁺) 66.2, ¹H NMR (400 MHz, DMSO-d₆) δ 13.58 (s, 1H), 10.70 (s, 1H), 10.21 (d, J=3.0 Hz, 2H), 8.98 (s, 2H), 8.45 (dd, J=5.1, 0.8 Hz, 1H), 8.43-8.39 (m, 1H), 7.92 (d, J=1.8 Hz, 1H), 7.74 (dd, J=8.1, 1.9 Hz, 1H), 7.66 (d, J=8.1 Hz, 1H), 7.47 (dd, J=5.1, 1.5 Hz, 1H), 7.38 (dd, J=8.5, 2.5 Hz, 1H), 7.33 (dd, J=8.7, 5.0 Hz, 1H), 7.00 (ddd, J=10.1, 8.6, 2.5 Hz, 1H), 6.17 (d, J=2.7 Hz, 1H), 2.61 (s, 3H), 2.26 (s, 3H).

### Example 3

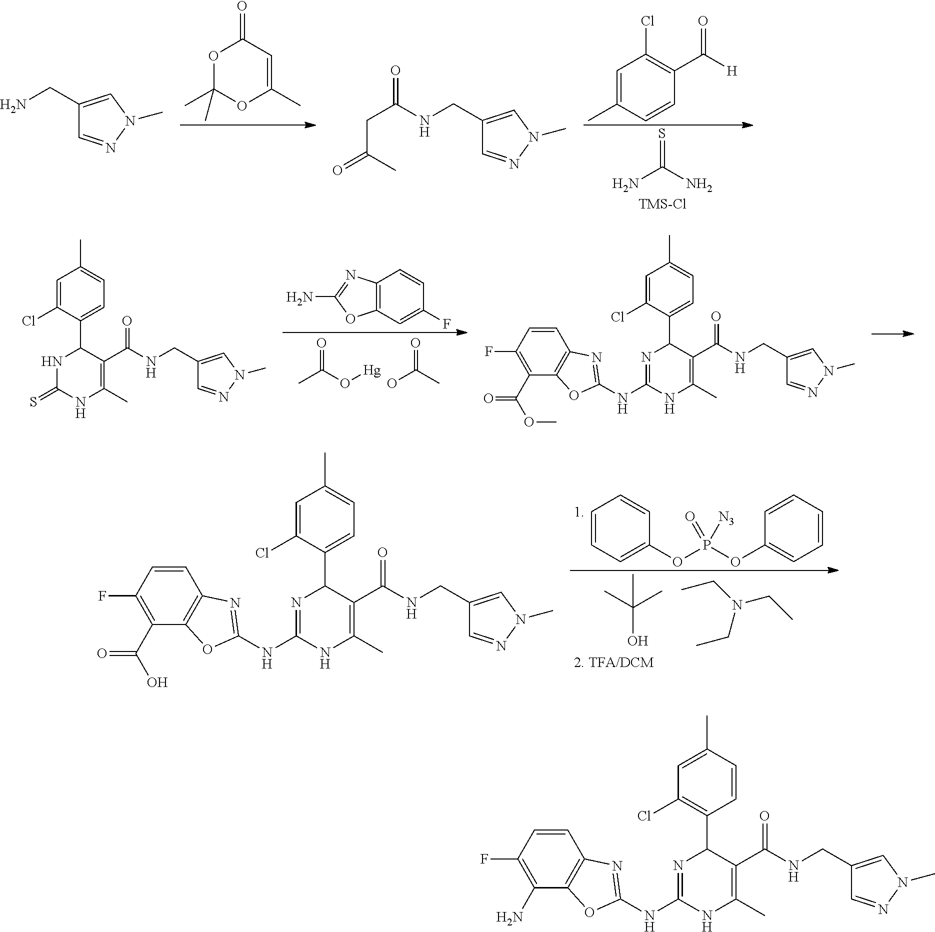

Step 1. The mixture of 2,2,6-trimethyl-4H-1,3-dioxin-4-one (2.37 mL, 17.99 mmol) and (1-methyl-1H-pyrazol-4-yl)methanamine (2 g, 17.99 mmol) in p-Xylene (22 mL) was sealed and heated 150° C. for 30 min in microwave. The solvent was removed. The crude product was purified by ISCO (0-20%, MeOH/EtOAc) to give desired product (1.94 g, 55.2%). MS m/z (M+H⁺) 196.1.

Step 2. To a solution of N-((1-methyl-1H-pyrazol-4-yl)methyl)-3-oxobutanamide (1.5 g, 7.68 mmol), 2-chloro-4-methylbenzaldehyde (1.188 g, 7.68 mmol) and thiourea (0.585 g, 7.68 mmol) in ACN (25 mL) and DMF (12.50 mL) was added dropwise TMS-Cl (0.982 mL, 7.68 mmol) at R.T. The mixture was stirred at 80° C. overnight. The reaction mixture was poured onto crushed ice and stirred until all ice had melted. The mixture was extracted with EtOAc (2×). The organic layer was washed with sat. NaHCO₃ (3×), and dried over MgSO₄, and concentrated. The crude product was purified by ISCO (0-10%, MeOH/DCM) to give desired product (2.2 g, 73%). MS m/z (M+H⁺) 390.2.

Step 3. The mixture of 4-(2-chloro-4-methylphenyl)-6-methyl-N-((1-methyl-1H-pyrazol-4-yl)methyl)-2-thioxo-1,2,3,4-tetrahydropyrimidine-5-carboxamide (0.68 g, 1.744 mmol), methyl 2-amino-6-fluorobenzo[d]oxazole-7-carboxylate (0.367 g, 1.744 mmol) and mercuric acetate (0.834 g, 2.62 mmol) in DCM (10 mL) and DMF (2.0 mL) in the sealed tube was stirred at 80° C. for 72 hr. The reaction was diluted with EtOAc and filtered throught Celite and washed with EtOAc. The filtrate was concentrated. EtOAc was added to the residue, the organic layer was washed with Sat. NaHCO₃ (2×) and brine, dried over MgSO₄ and concentrated. DCM was added to residue. The solid was filtered and washed with DCM. The filtrate was purified by ISCO (0-20%, MeOH/EtOAc) to give desired product (0.11 g, 11%). MS m/z (M+H⁺) 566.0.

Step 4. To a solution of methyl 2-((4-(2-chloro-4-methylphenyl)-6-methyl-5-(((1-methyl-1H-pyrazol-4-yl)methyl)carbamoyl)-1,4-dihydropyrimidin-2-yl)amino)-6-fluorobenzo[d]oxazole-7-carboxylate (100 mg, 0.177 mmol) in THF (1.5 mL) was added LiOH (0.707 mL, 0.353 mmol)(0.5M). The mixture was stirred at R.T. for 4 hr. The solvent was evaporated under vacuum. Water was added to the residue. The mixture was extracted with EtOAc, and the water layer pH was adjusted to 2 with 1 N HCl. The solid was filtered, and dried. The crude product was used in the next reaction without further purification (44 mg, 37%). MS m/z (M+H⁺) 552.2. (Compound 128)

Step 5. To the solution of 2-((4-(2-chloro-4-methylphenyl)-6-methyl-5-(((1-methyl-1H-pyrazol-4-yl)methyl)carbamoyl)-1,4-dihydropyrimidin-2-yl)amino)-6-fluorobenzo[d]oxazole-7-carboxylic acid (44 mg, 0.080 mmol) in Dioxane (1 mL) were added 2-methylpropan-2-ol (0.061 mL, 0.638 mmol), TEA (0.044 mL, 0.319 mmol) and diphenyl phosphorazidate (0.018 mL, 0.084 mmol) at R.T. The mixture was stirred at 100° C. for 5 hr. Upon cooling, the cloudy mixture was filtered, and washed with EtOAc. The filtrate was evaporated under vacuum. To a solution of the residue (40 mg, 0.064 mmol) in DCM (1 mL) was added TFA (0.4 mL, 5.19 mmol). The mixture was stirred at R.T. for 2 hr. The solvent was removed. The crude product was purified (in acid condition) to give desired product as a TFA salt. MS m/z (M+H⁺) 523.2, ¹H NMR (400 MHz, DMSO-d6) δ 10.01 (s, 1H), 9.74 (s, 1H), 8.15 (t, J=5.8 Hz, 1H), 7.31-7.26 (m, 2H), 7.24 (s, 1H), 7.11 (d, J=9.1 Hz, 2H), 6.83 (dd, J=12.2, 8.5 Hz, 1H), 6.46 (dd, J=8.5, 3.9 Hz, 1H), 5.90-5.84 (m, 1H), 4.03 (t, J=5.2 Hz, 2H), 3.69 (s, 3H), 2.24 (s, 3H), 2.11 (s, 3H). (Compound 126)

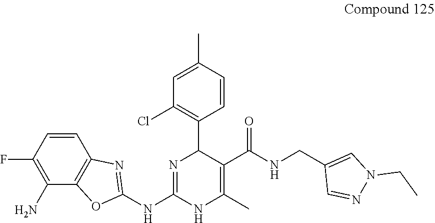 [SMILES: CCn1cc(CNC(=O)C2=C(C)NC(Nc3nc4ccc(F)c(N)c4o3)=NC2c2ccc(C)cc2Cl)cn1]

The title compound 2-((7-amino-6-fluorobenzo[d]oxazol-2-yl)amino)-4-(2-chloro-4-methylphenyl)-N-((1-ethyl-1H-pyrazol-4-yl)methyl)-6-methyl-1,4-dihydropyrimidine-5-carboxamide was prepared according to Example 3 using (1-ethyl-1H-pyrazol-4-yl)methanamine in Step 1 as starting material. MS m/z (M+H⁺) 537.2, ¹H NMR (400 MHz, DMSO-d₆) δ 10.02 (s, 1H), 9.73 (s, 1H), 8.16 (t, J=5.7 Hz, 1H), 7.32-7.27 (m, 2H), 7.26 (s, 1H), 7.12 (d, J=4.0 Hz, 2H), 6.83 (dd, J=12.2, 8.5 Hz, 1H), 6.46 (dd, J=8.5, 3.9 Hz, 1H), 5.91-5.86 (m, 1H), 4.04 (dd, J=5.6, 3.1 Hz, 2H), 3.97 (q, J=7.2 Hz, 2H), 2.24 (s, 3H), 2.11 (s, 3H), 1.25 (t, J=7.3 Hz, 3H).

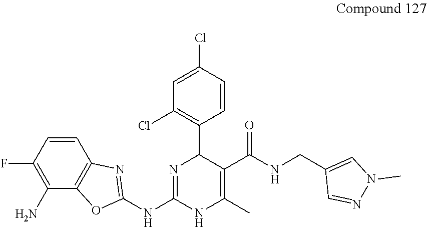 [SMILES: CC1=C(C(=O)NCc2cnn(C)c2)C(c2ccc(Cl)cc2Cl)N=C(Nc2nc3ccc(F)c(N)c3o2)N1]

The title compound 2-((7-amino-6-fluorobenzo[d]oxazol-2-yl)amino)-4-(2,4-dichlorophenyl)-6-methyl-N-((1-methyl-1H-pyrazol-4-yl)methyl)-1,4-dihydropyrimidine-5-carboxamide was prepared according to Example 3 using (1-methyl-1H-pyrazol-4-yl)methanamine in Step 1 and 2,4-dichlorobenzaldehyde in Step 2 as starting material. MS m/z (M+H⁺) 543.0, ¹H NMR (400 MHz, Methanol-d₄) δ 7.44 (d, J=2.1 Hz, 1H), 7.37 (d, J=8.4 Hz, 1H), 7.27 (dd, J=8.4, 2.1 Hz, 1H), 7.22 (s, 2H), 6.80 (dd, J=12.0, 8.6 Hz, 1H), 6.58 (dd, J=8.6, 4.1 Hz, 1H), 5.97 (s, 1H), 4.15 (s, 2H), 3.77 (s, 3H), 2.17-2.11 (m, 3H). 3× (NH) and NH₂ did not shown in the NMR.

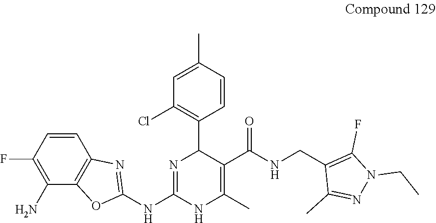 [SMILES: CCn1nc(C)c(CNC(=O)C2=C(C)NC(Nc3nc4ccc(F)c(N)c4o3)=NC2c2ccc(C)cc2Cl)c1F]

The title compound 2-((7-amino-6-fluorobenzo[d]oxazol-2-yl)amino)-4-(2-chloro-4-methylphenyl)-N-((1-ethyl-5-fluoro-3-methyl-1H-pyrazol-4-yl)methyl)-6-methyl-1,4-dihydropyrimidine-5-carboxamide was prepared according to Example 3 using (1-ethyl-5-fluoro-3-methyl-1H-pyrazol-4-yl)methanamine hydrochloride in Step 1 as starting material. MS m/z (M+H⁺) 569.2

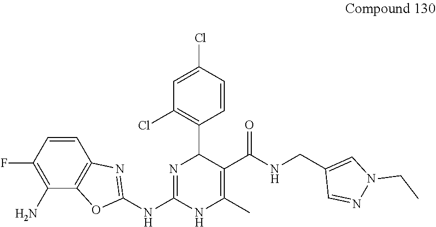 [SMILES: CCn1cc(CNC(=O)C2=C(C)NC(Nc3nc4ccc(F)c(N)c4o3)=NC2c2ccc(Cl)cc2Cl)cn1]

The title compound 2-((7-amino-6-fluorobenzo[d]oxazol-2-yl)amino)-4-(2,4-dichlorophenyl)-N-((1-ethyl-1H-pyrazol-4-yl)methyl)-6-methyl-1,4-dihydropyrimidine-5-carboxamide was prepared according to Example 3 using (1-ethyl-1H-pyrazol-4-yl)methanamine in Step 1 and 2,4-dichlorobenzaldehyde in Step 2 as starting material. MS m/z (M+H⁺) 557.0, ¹H NMR (400 MHz, Methanol-d₄) δ 8.31-8.24 (m, 1H), 7.46 (d, J=2.1 Hz, 1H), 7.40 (d, J=8.4 Hz, 1H), 7.33-7.16 (m, 4H), 6.82 (dd, J=12.0, 8.6 Hz, 1H), 6.58 (dd, J=8.6, 4.0 Hz, 1H), 6.02-5.97 (m, 1H), 4.20-4.10 (m, 2H), 4.06 (q, J=7.3 Hz, 3H), 2.15 (s, 3H), 1.43-1.32 (m, 3H).

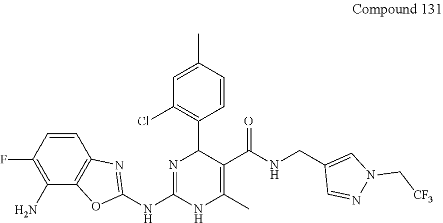 [SMILES: CC1=C(C(=O)NCc2cnn(CC(F)(F)F)c2)C(c2ccc(C)cc2Cl)N=C(Nc2nc3ccc(F)c(N)c3o2)N1]

The title compound 2-((7-amino-6-fluorobenzo[d]oxazol-2-yl)amino)-4-(2-chloro-4-methylphenyl)-6-methyl-N-((1-(2,2,2-trifluoroethyl)-1H-pyrazol-4-yl)methyl)-1,4-dihydropyrimidine-5-carboxamide was prepared according to Example 3 using (1-(2,2,2-trifluoroethyl)-1H-pyrazol-4-yl)methanamine in Step 1 as starting material. MS m/z (M+H⁺) 591.2

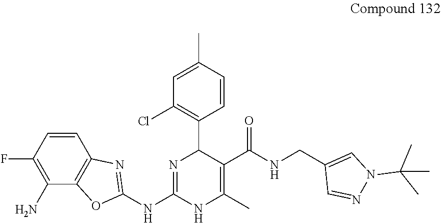 [SMILES: CC1=C(C(=O)NCc2cnn(C(C)(C)C)c2)C(c2ccc(C)cc2Cl)N=C(Nc2nc3ccc(F)c(N)c3o2)N1]

The title compound 2-((7-amino-6-fluorobenzo[d]oxazol-2-yl)amino)-N-((1-(tert-butyl)-1H-pyrazol-4-yl)methyl)-4-(2-chloro-4-methylphenyl)-6-methyl-1,4-dihydropyrimidine-5-carboxamide was prepared according to Example 3 using (1-(tert-butyl)-1H-pyrazol-4-yl)methanamine, 2HCl in Step 1 as starting material. MS m/z (M+H⁺) 565.1, ¹H NMR (400 MHz, Methanol-d₄) δ 7.42 (s, 1H), 7.31 (d, J=7.9 Hz, 1H), 7.27-7.21 (m, 2H), 7.12-7.05 (m, 1H), 6.80 (dd, J=12.0, 8.6 Hz, 1H), 6.59 (dd, J=8.6, 4.0 Hz, 1H), 6.02-5.98 (m, 1H), 4.18 (d, J=2.1 Hz, 2H), 2.28 (s, 3H), 2.20-2.14 (m, 3H), 1.47 (d, J=1.5 Hz, 9H).

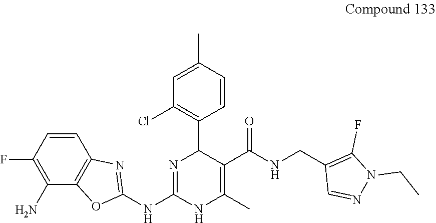 [SMILES: CCn1ncc(CNC(=O)C2=C(C)NC(Nc3nc4ccc(F)c(N)c4o3)=NC2c2ccc(C)cc2Cl)c1F]

The title compound 2-((7-amino-6-fluorobenzo[d]oxazol-2-yl)amino)-4-(2-chloro-4-methylphenyl)-N-((1-ethyl-5-fluoro-1H-pyrazol-4-yl)methyl)-6-methyl-1,4-dihydropyrimidine-5-carboxamide was prepared according to Example 3 using (1-ethyl-5-fluoro-1H-pyrazol-4-yl)methanamine hydrochloride in Step 1 as starting material. MS m/z (M+H⁺) 555.2, ¹H NMR (400 MHz, DMSO-d₆) δ 10.03 (s, 1H), 9.73 (s, 1H), 8.17 (t, J=5.6 Hz, 1H), 7.29-7.21 (m, 2H), 7.12-7.06 (m, 2H), 6.82 (dd, J=12.2, 8.6 Hz, 1H), 6.46 (dd, J=8.5, 3.8 Hz, 1H), 5.85 (s, 1H), 5.14 (s, 2H), 3.93 (p, J=7.3, 6.3 Hz, 4H), 2.23 (s, 3H), 2.11 (s, 3H), 1.24 (t, J=7.3 Hz, 3H).

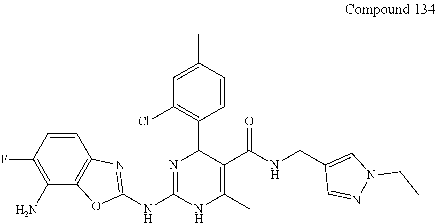 [SMILES: CCn1cc(CNC(=O)C2=C(C)NC(Nc3nc4ccc(F)c(N)c4o3)=NC2c2ccc(C)cc2Cl)cn1]

The title compound (S)-2-((7-amino-6-fluorobenzo[d]oxazol-2-yl)amino)-4-(2-chloro-4-methylphenyl)-N-((1-ethyl-1H-pyrazol-4-yl)methyl)-6-methyl-1,4-dihydropyrimidine-5-carboxamide was prepared according to Example 3 using (1-ethyl-1H-pyrazol-4-yl)methanamine in Step 1 as starting material. The final product was submitted for Chiral Separation. MS m/z (M+H⁺) 537.1, ¹H NMR (400 MHz, Methanol-d₄) δ 7.30 (d, J=7.9 Hz, 1H), 7.24 (d, J=2.4 Hz, 2H), 7.19 (s, 1H), 7.09 (dd, J=8.1, 1.8 Hz, 1H), 6.80 (td, J=9.5, 8.6, 1.9 Hz, 1H), 6.59 (dd, J=8.6, 4.0 Hz, 1H), 6.00 (s, 1H), 4.17 (s, 2H), 4.04 (q, J=7.3 Hz, 2H), 2.29 (s, 3H), 2.17 (s, 3H), 1.35 (t, J=7.3 Hz, 3H).

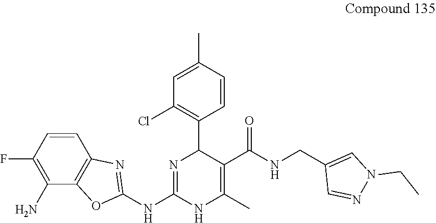 [SMILES: CCn1cc(CNC(=O)C2=C(C)NC(Nc3nc4ccc(F)c(N)c4o3)=NC2c2ccc(C)cc2Cl)cn1]

The title compound (R)-2-((7-amino-6-fluorobenzo[d]oxazol-2-yl)amino)-4-(2-chloro-4-methylphenyl)-N-((1-ethyl-1H-pyrazol-4-yl)methyl)-6-methyl-1,4-dihydropyrimidine-5-carboxamide was prepared according to Example 3 using (1-ethyl-1H-pyrazol-4-yl)methanamine in Step 1 as starting material. The final product was submitted for Chiral Separation. MS m/z (M+H⁺) 537.1, ¹H NMR (400 MHz, Methanol-d₄) δ 7.31 (d, J=8.0 Hz, 1H), 7.24 (d, J=2.9 Hz, 2H), 7.19 (s, 1H), 7.16-7.06 (m, 1H), 6.80 (dd, J=12.0, 8.6 Hz, 1H), 6.59 (dd, J=8.5, 4.0 Hz, 1H), 6.02-5.97 (m, 1H), 4.17 (s, 2H), 4.04 (q, J=7.3 Hz, 2H), 2.29 (s, 3H), 2.17 (d, J=1.3 Hz, 3H), 1.35 (t, J=7.3 Hz, 3H).

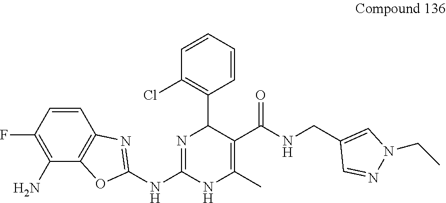 [SMILES: CCn1cc(CNC(=O)C2=C(C)NC(Nc3nc4ccc(F)c(N)c4o3)=NC2c2ccccc2Cl)cn1]

The title compound 2-((7-amino-6-fluorobenzo[d]oxazol-2-yl)amino)-4-(2-chlorophenyl)-N-((1-ethyl-1H-pyrazol-4-yl)methyl)-6-methyl-1,4-dihydropyrimidine-5-carboxamide was prepared according to Example 3 using (1-ethyl-1H-pyrazol-4-yl)methanamine in Step 1 and using 2-chlorobenzaldehyde in Step 2 as starting material. The final product was submitted for Chiral Separation. MS m/z (M+H⁺) 523.1, ¹H NMR (400 MHz, DMSO-d₆) δ 10.08 (s, 1H), 9.75 (s, 1H), 8.17 (t, J=5.7 Hz, 1H), 7.47 (dd, J=5.8, 3.4 Hz, 1H), 7.44-7.40 (m, 1H), 7.36-7.29 (m, 2H), 7.28 (s, 1H), 7.11 (s, 1H), 6.83 (dd, J=12.2, 8.5 Hz, 1H), 6.48 (dd, J=8.5, 4.0 Hz, 1H), 5.96-5.90 (m, 1H), 5.20 (s, 2H), 4.05 (d, J=5.6 Hz, 2H), 3.98 (q, J=7.3 Hz, 2H), 2.14 (s, 3H), 1.26 (t, J=7.3 Hz, 3H).

### Example 4

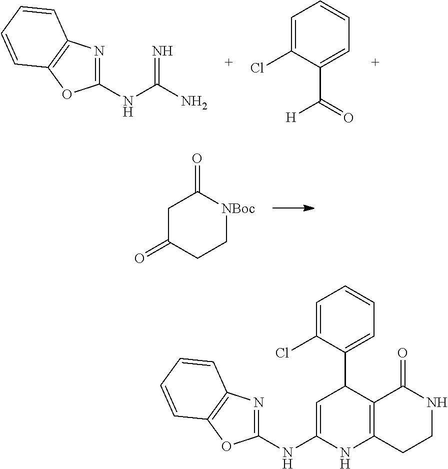 [SMILES: N=C(N)Nc1nc2ccccc2o1 ; O=Cc1ccccc1Cl ; CC(C)(C)OC(=O)N1CCC(=O)CC1=O ; O=C1NCCC2=C1C(c1ccccc1Cl)C=C(Nc1nc3ccccc3o1)N2]

A mixture of tert-butyl 2,4-dioxopiperidine-1-carboxylate (58.1 mg, 0.272 mmol), 1-(benzo[d]oxazol-2-yl)guanidine (40 mg, 0.227 mmol), and 2-chlorobenzaldehyde (30.6 μL, 0.272 mmol) was heated at 120° C. for 1 hr in a sealed tube. The crude mixture was purified by chromatography (5:95 to 20:80 MeOH/DCM) to afford the crude product. The crude product was diluted with DMSO and purified by reverse phase chromatography (Method Acidic Standard Gradient) to afford as a TFA salt, Compound 245 (2.1 mg, 1.8% yield). MS: m/z (M+H⁺) 394.0; ¹H NMR (400 MHz, DMSO-d₆) δ 10.41 (s, 1H), 10.33 (s, 1H), 7.52-7.47 (m, 1H), 7.41-7.28 (m, 5H), 7.24 (d, J=3.3 Hz, 1H), 7.15 (td, J=7.6, 1.2 Hz, 1H), 7.08 (td, J=7.7, 1.3 Hz, 1H), 5.88 (s, 1H), 3.52-3.21 (m, 2H), 2.60 (q, J=7.4, 5.5 Hz, 2H). (Compound 245)

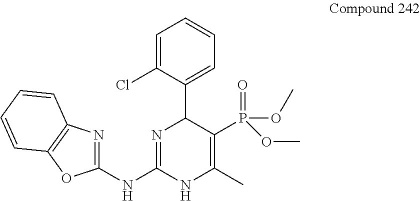 [SMILES: COP(=O)(OC)C1=C(C)NC(Nc2nc3ccccc3o2)=NC1c1ccccc1Cl]

The title compound dimethyl (2-(benzo[d]oxazol-2-ylamino)-4-(2-chlorophenyl)-6-methyl-1,4-dihydropyrimidin-5-yl)phosphonate was prepared according to Example 4 using dimethyl (2-oxopropyl)phosphonate as starting material. MS m/z (M+H⁺) 447.0.

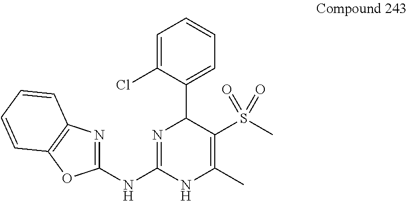 [SMILES: CC1=C(S(C)(=O)=O)C(c2ccccc2Cl)N=C(Nc2nc3ccccc3o2)N1]

The title compound N-(4-(2-chlorophenyl)-6-methyl-5-(methylsulfonyl)-1,4-dihydropyrimidin-2-yl)benzo[d]oxazol-2-amine was prepared according to Example 4 using 1-(methylsulfonyl)propan-2-one as starting material. MS m/z (M+H⁺) 417.0; ¹H NMR (400 MHz, DMSO-d₆) δ 10.62 (s, 1H), 10.49 (s, 1H), 7.50 (dd, J=7.3, 1.7 Hz, 1H), 7.42-7.31 (m, 5H), 7.17 (td, J=7.6, 1.3 Hz, 1H), 7.11 (td, J=7.7, 1.4 Hz, 1H), 5.98 (d, J=3.5 Hz, 1H), 3.03 (s, 3H), 2.42 (s, 3H).

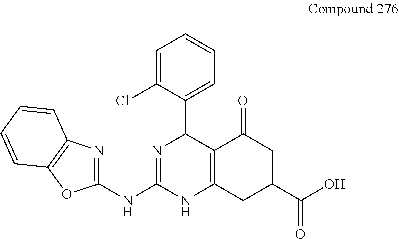 [SMILES: O=C1CC(C(=O)O)CC2=C1C(c1ccccc1Cl)N=C(Nc1nc3ccccc3o1)N2]

The title compound 2-(benzo[d]oxazol-2-ylamino)-4-(2-chlorophenyl)-5-oxo-1,4,5,6,7,8-hexahydroquinazoline-7-carboxylic acid was prepared according to Example 4 using 3,5-dioxocyclohexanecarboxylic acid as starting material. MS m/z (M+H⁺) 437.1.

### Example 5

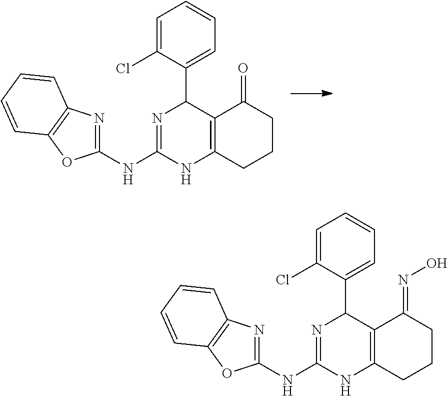 [SMILES: O=C1CCCC2=C1C(c1ccccc1Cl)N=C(Nc1nc3ccccc3o1)N2 ; O/N=C1\CCCC2=C1C(c1ccccc1Cl)N=C(Nc1nc3ccccc3o1)N2]

A solution of 2-(benzo[d]oxazol-2-ylamino)-4-(2-chlorophenyl)-4,6,7,8-tetrahydroquinazolin-5(1H)-one (20 mg, 0.051 mmol) in EtOH (407 μL) was treated with hydroxylamine hydrochloride (35.4 mg, 0.509 mmol) and sodium acetate (125 mg, 1.527 mmol) in water (0.2 mL). This was stirred at R.T. for 1 h and treated with additional DMSO (0.4 mL) to dissolve SM. This solution was heated to 90° C. for 3 days. The crude mixture was diluted with ethyl acetate, washed with brine (3×), dried over MgSO₄, and concentrated under reduced pressure. The crude product was diluted with DMSO and purified by reverse phase chromatography (Method Acidic Standard Gradient) to afford as a TFA salt, (2.1 mg, 7.9% yield). MS m/z (M+H⁺) 408.1. (Compound 244)

### Example 6

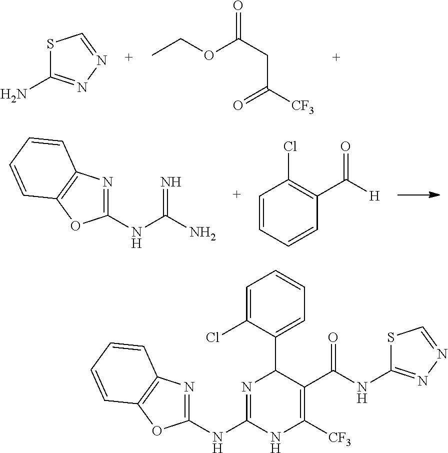

A solution of 1,3,4-thiadiazol-2-amine (31.0 mg, 0.307 mmol) in acetonitrile (511 μL) was treated with ethyl 4,4,4-trifluoro-3-oxobutanoate (56.4 mg, 0.307 mmol). The mixture was heated to 150° C. for 30 minutes and then, concentrated in the blowdown unit and placed on high vacuum.

The crude ketoamide was treated 1-(benzo[d]oxazol-2-yl)guanidine (45 mg, 0.255 mmol), sonicated, and then with 2-chlorobenzaldehyde (34.5 μL, 0.307 mmol). The mixture was heated at 120° C. for 1 h in a heating rack. The crude product was diluted with DMSO and purified by reverse phase chromatography (Method Acidic Standard Gradient) to afford as a TFA salt, (3.6 mg, 2.2% yield). MS m/z (M+H⁺) 519.7. Compound 246

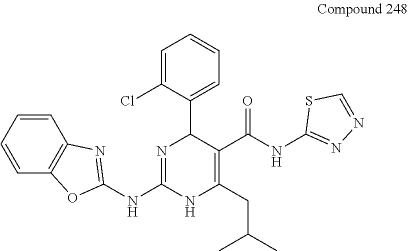 [SMILES: CC(C)CC1=C(C(=O)Nc2nncs2)C(c2ccccc2Cl)N=C(Nc2nc3ccccc3o2)N1]

The title compound 2-(benzo[d]oxazol-2-ylamino)-4-(2-chlorophenyl)-6-isobutyl-N-(1,3,4-thiadiazol-2-yl)-1,4-dihydropyrimidine-5-carboxamide was prepared according to Example 6 using ethyl 4-methyl-3-oxopentanoate as starting material. MS m/z (M+H⁺) 508.1.

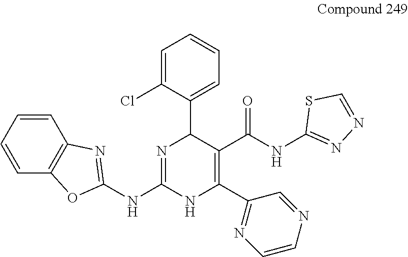 [SMILES: O=C(Nc1nncs1)C1=C(c2cnccn2)NC(Nc2nc3ccccc3o2)=NC1c1ccccc1Cl]

The title compound 2-(benzo[d]oxazol-2-ylamino)-4-(2-chlorophenyl)-6-(pyrazin-2-yl)-N-(1,3,4-thiadiazol-2-yl)-1,4-dihydropyrimidine-5-carboxamide was prepared according to Example 6 using ethyl 3-oxo-3-(pyrazin-2-yl)propanoate as starting material. MS m/z (M+H⁺) 530.0.

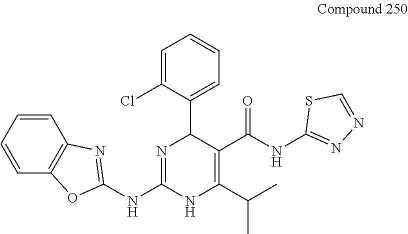 [SMILES: CC(C)C1=C(C(=O)Nc2nncs2)C(c2ccccc2Cl)N=C(Nc2nc3ccccc3o2)N1]

The title compound 2-(benzo[d]oxazol-2-ylamino)-4-(2-chlorophenyl)-6-isopropyl-N-(1,3,4-thiadiazol-2-yl)-1,4-dihydropyrimidine-5-carboxamide was prepared according to Example 6 using ethyl 4-methyl-3-oxopentanoate as starting material. MS m/z (M+H⁺) 494.1.

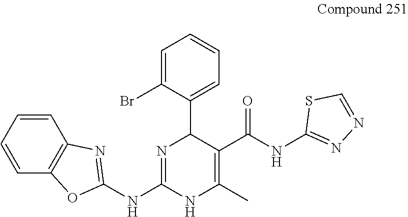 [SMILES: CC1=C(C(=O)Nc2nncs2)C(c2ccccc2Br)N=C(Nc2nc3ccccc3o2)N1]

The title compound 2-(benzo[d]oxazol-2-ylamino)-4-(2-bromophenyl)-6-methyl-N-(1,3,4-thiadiazol-2-yl)-1,4-dihydropyrimidine-5-carboxamide was prepared according to Example 6 using ethyl 3-oxobutanoate and 2-bromobenzaldehyde as starting material. MS m/z (M+H⁺) 509.6/511.

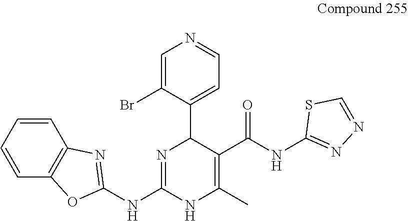 [SMILES: CC1=C(C(=O)Nc2nncs2)C(c2ccncc2Br)N=C(Nc2nc3ccccc3o2)N1]

The title compound 2-(benzo[d]oxazol-2-ylamino)-4-(3-bromopyridin-4-yl)-6-methyl-N-(1,3,4-thiadiazol-2-yl)-1,4-dihydropyrimidine-5-carboxamide was prepared according to Example 6 using ethyl 3-oxobutanoate and 3-bromoisonicotinaldehyde as starting material. MS m/z (M+H⁺) 510.6/512.

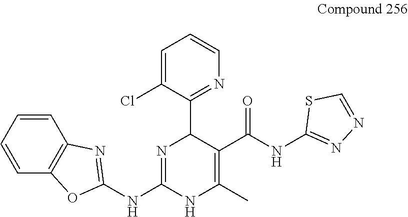 [SMILES: CC1=C(C(=O)Nc2nncs2)C(c2ncccc2Cl)N=C(Nc2nc3ccccc3o2)N1]

The title compound 2-(benzo[d]oxazol-2-ylamino)-4-(3-chloropyridin-2-yl)-6-methyl-N-(1,3,4-thiadiazol-2-yl)-1,4-dihydropyrimidine-5-carboxamide was prepared according to Example 6 using ethyl 3-oxobutanoate and 3-chloropicolinaldehyde as starting material. MS m/z (M+H⁺) 467.0.

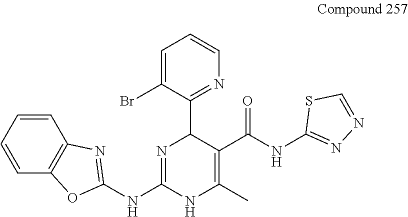 [SMILES: CC1=C(C(=O)Nc2nncs2)C(c2ncccc2Br)N=C(Nc2nc3ccccc3o2)N1]

The title compound 2-(benzo[d]oxazol-2-ylamino)-4-(3-bromopyridin-2-yl)-6-methyl-N-(1,3,4-thiadiazol-2-yl)-1,4-dihydropyrimidine-5-carboxamide was prepared according to Example 6 using ethyl 3-oxobutanoate and 3-bromopicolinaldehyde as starting material. MS m/z (M+H⁺) 511.0/513.

### Example 7

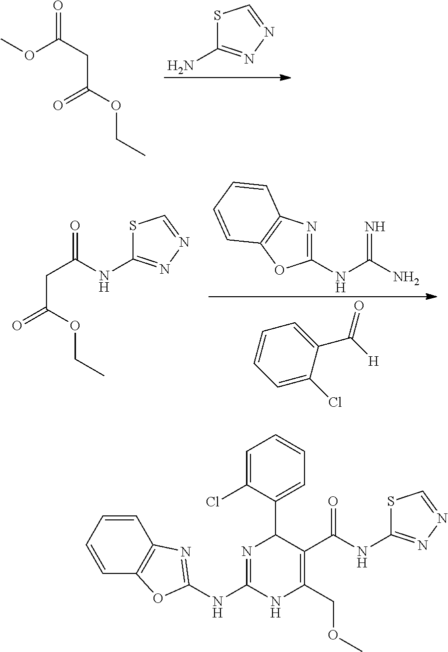

Step 1. The mixture of 1,3,4-thiadiazol-2-amine (40 mg, 0.396 mmol) and methyl 4-methoxy-3-oxobutanoate (57.8 mg, 0.396 mmol) in acetonitrile (2 mL) was sealed and heated 140° C. for 30 min in microwave. The solvent was removed. The crude product was used in the next reaction without further purification. MS m/z (M+H⁺) 216.1.

Step 2. The mixture of 4-methoxy-3-oxo-N-(1,3,4-thiadiazol-2-yl)butanamide (0.085 g, 0.395 mmol), 2-chlorobenzaldehyde (0.044 mL, 0.395 mmol) and 1-(benzo[d]oxazol-2-yl)guanidine (0.058 g, 0.329 mmol) was sealed in microwave tube and sonicated/stirred to homogeneity. Then the mixture was heated at 120° C. for 1.5 h. Dissolve in DMSO. The crude product was purified. MS m/z (M+H⁺) 496.1. (Compound 247)

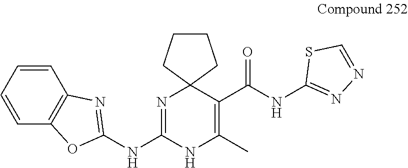 [SMILES: CC1=C(C(=O)Nc2nncs2)C2(CCCC2)N=C(Nc2nc3ccccc3o2)N1]

The title compound 7-(benzo[d]oxazol-2-ylamino)-9-methyl-N-(1,3,4-thiadiazol-2-yl)-6,8-diazaspiro[4.5]deca-6,9-diene-10-carboxamide was prepared according to Example 7 using ethyl 3-oxobutanoate in Step 1 and using cyclopentanone in Step 2 as starting material. MS m/z (M+H⁺) 410.1.

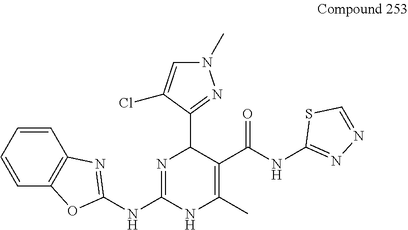 [SMILES: CC1=C(C(=O)Nc2nncs2)C(c2nn(C)cc2Cl)N=C(Nc2nc3ccccc3o2)N1]

The title compound 2-(benzo[d]oxazol-2-ylamino)-4-(4-chloro-1-methyl-1H-pyrazol-3-yl)-6-methyl-N-(1,3,4-thiadiazol-2-yl)-1,4-dihydropyrimidine-5-carboxamide was prepared according to Example 7 using ethyl 3-oxobutanoate in Step 1 and using 4-chloro-1-methyl-1H-pyrazole-3-carbaldehyde in Step 2 as starting material. MS m/z (M+H⁺) 470.1, ¹H NMR (400 MHz, DMSO-d₆) δ 12.24 (s, 1H), 10.33 (s, 1H), 9.95 (t, J=2.5 Hz, 1H), 7.57-7.47 (m, 1H), 7.44-7.33 (m, 2H), 7.31-7.19 (m, 1H), 7.13 (dtd, J=26.4, 7.6, 1.3 Hz, 2H), 6.09 (d, J=3.1 Hz, 1H), 3.69 (s, 3H), 2.20 (s, 3H).

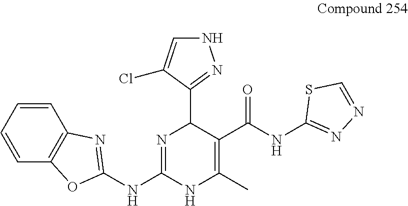 [SMILES: CC1=C(C(=O)Nc2nncs2)C(c2n[nH]cc2Cl)N=C(Nc2nc3ccccc3o2)N1]

The title compound 2-(benzo[d]oxazol-2-ylamino)-4-(4-chloro-1H-pyrazol-3-yl)-6-methyl-N-(1,3,4-thiadiazol-2-yl)-1,4-dihydropyrimidine-5-carboxamide was prepared according to Example 7 using ethyl 3-oxobutanoate in Step 1 and using 4-chloro-1H-pyrazole-3-carbaldehyde in Step 2 as starting material. MS m/z (M+H⁺) 456.1, ¹H NMR (400 MHz, DMSO-d₆) δ 13.48 (s, 1H), 12.93 (s, 1H), 9.19 (s, 1H), 8.14-7.93 (m, 1H), 7.53 (ddd, J=7.7, 3.4, 1.3 Hz, 2H), 7.39 (t, J=8.0 Hz, 2H), 7.33-6.97 (m, 2H), 6.13 (s, 1H), 2.22 (m, 3H).

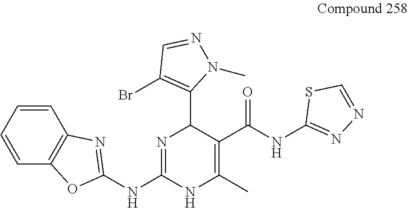 [SMILES: CC1=C(C(=O)Nc2nncs2)C(c2c(Br)cnn2C)N=C(Nc2nc3ccccc3o2)N1]

The title compound 2-(benzo[d]oxazol-2-ylamino)-4-(4-bromo-1-methyl-1H-pyrazol-5-yl)-6-methyl-N-(1,3,4-thiadiazol-2-yl)-1,4-dihydropyrimidine-5-carboxamide was prepared according to Example 7 using ethyl 3-oxobutanoate in Step 1 and using 4-bromo-1-methyl-1H-pyrazole-5-carbaldehyde in Step 2 as starting material. MS m/z (M+H⁺) 514.0/516, ¹H NMR (400 MHz, DMSO-d₆) δ 12.43 (s, 1H), 10.42 (s, 1H), 9.63 (s, 1H), 7.61-7.49 (m, 1H), 7.46-7.32 (m, 1H), 7.32-7.18 (m, 2H), 7.19-6.99 (m, 2H), 6.19 (s, 1H), 3.91 (s, 3H), 2.14 (s, 3H).

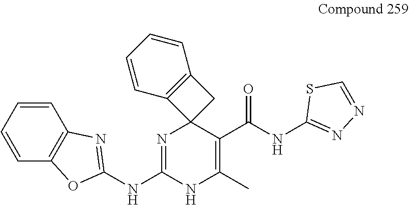 [SMILES: CC1=C(C(=O)Nc2nncs2)C2(Cc3ccccc32)N=C(Nc2nc3ccccc3o2)N1]

The title compound 2′-(benzo[d]oxazol-2-ylamino)-6′-methyl-N-(1,3,4-thiadiazol-2-yl)-1′H-spiro[bicyclo[4.2.0]octa[1(6),2,4]triene-7,4′-pyrimidine]-5′-carboxamide was prepared according to Example 7 using ethyl 3-oxobutanoate in Step 1 and using bicyclo[4.2.0]octa-1,3,5-trien-7-one in Step 2 as starting material. MS m/z (M+H⁺) 444.1, ¹H NMR (400 MHz, DMSO-d₆) δ 13.0 (s, 1H), 11.73 (s, 1H). 10.34 (s, 1H), 9.97 (s, 1H), 9.15 (d, J=0.8 Hz, 1H), 7.58-7.46 (m, 2H), 7.30-7.17 (m, 5H), 2.54 (d, J=14.4 Hz, 1H), 2.49 (d, J=14.4 Hz, 1H), 2.24 (s, 3H).

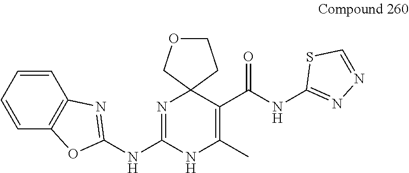 [SMILES: CC1=C(C(=O)Nc2nncs2)C2(CCOC2)N=C(Nc2nc3ccccc3o2)N1]

The title compound 7-(benzo[d]oxazol-2-ylamino)-9-methyl-N-(1,3,4-thiadiazol-2-yl)-2-oxa-6,8-diazaspiro[4.5]deca-6,9-diene-10-carboxamide was prepared according to Example 7 using ethyl 3-oxobutanoate in Step 1 and using dihydrofuran-3(2H)-one in Step 2 as starting material. MS m/z (M+H⁺) 412.2, ¹H NMR (400 MHz, DMSO-d₆) δ 12.71 (s, 1H), 10.20 (s, 1H), 9.97 (s, 1H), 7.48-7.32 (m, 2H), 7.24-7.00 (m, 3H), 4.00 (q, J=7.9 Hz, 2H), 3.89-3.69 (m, 2H), 2.16-2.04 (m, 2H), 1.97 (s, 3H).

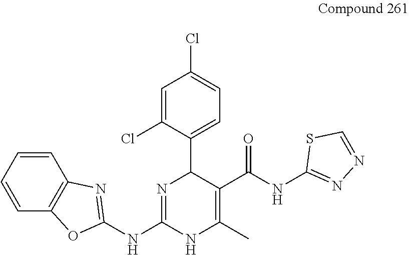 [SMILES: CC1=C(C(=O)Nc2nncs2)C(c2ccc(Cl)cc2Cl)N=C(Nc2nc3ccccc3o2)N1]

The title compound 2-(benzo[d]oxazol-2-ylamino)-4-(2,4-dichlorophenyl)-6-methyl-N-(1,3,4-thiadiazol-2-yl)-1,4-dihydropyrimidine-5-carboxamide was prepared according to Example 7 using ethyl 3-oxobutanoate in Step 1 and using 2,4-dichlorobenzaldehyde in Step 2 as starting material. MS m/z (M+H⁺) 500.0/502.0, ¹H NMR (400 MHz, DMSO-d₆) δ 12.47 (s, 1H), 10.37 (d, J=14.4 Hz, 2H), 9.12 (s, 1H) 7.69-7.61 (m, 1H), 7.47-7.32 (m, 4H), 7.14 (dtd, J=28.6, 7.6, 1.3 Hz, 2H), 6.15 (dd, J=3.1, 1.0 Hz, 1H), 2.30 (s, 3H).

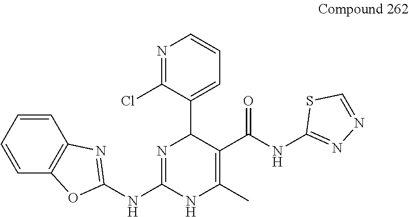 [SMILES: CC1=C(C(=O)Nc2nncs2)C(c2cccnc2Cl)N=C(Nc2nc3ccccc3o2)N1]

The title compound 2-(benzo[d]oxazol-2-ylamino)-4-(2-bromopyridin-3-yl)-6-methyl-N-(1,3,4-thiadiazol-2-yl)-1,4-dihydropyrimidine-5-carboxamide was prepared according to Example 7 using ethyl 3-oxobutanoate in Step 1 and using 2-bromonicotinaldehyde in Step 2 as starting material. MS m/z (M+H⁺) 511.0/513.0, ¹H NMR (400 MHz, DMSO-d₆) δ 10.55 (s, 1H), 10.41 (s, 1H), 9.83 (s, 1H), 9.17 (s, 1H), 8.57 (dd, J=4.8, 1.9 Hz, 1H), 8.42 (dd, J=4.8, 1.9 Hz, 1H), 8.29 (dd, J=4.7, 1.9 Hz, 1H), 7.65 (ddd, J=19.2, 7.6, 1.5 Hz, 1H), 7.58-7.42 (m, 1H), 7.43-7.36 (m, 1H), 7.36-7.13 (m, 1H), 5.72 (s, 1H), 2.27 (s, 3H).

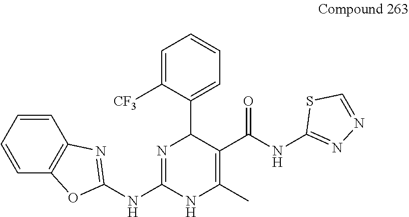 [SMILES: CC1=C(C(=O)Nc2nncs2)C(c2ccccc2C(F)(F)F)N=C(Nc2nc3ccccc3o2)N1]

The title compound 2-(benzo[d]oxazol-2-ylamino)-6-methyl-N-(1,3,4-thiadiazol-2-yl)-4-(2-(trifluoromethyl)phenyl)-1,4-dihydropyrimidine-5-carboxamide was prepared according to Example 7 using ethyl 3-oxobutanoate in Step 1 and using 2-(trifluoromethyl)benzaldehyde in Step 2 as starting material. MS m/z (M+H⁺) 500.1, ¹H NMR (400 MHz, DMSO-d₆) δ 12.46 (s, 1H), 10.34 (s, 1H), 10.26 (s, 1H), 9.09 (s, 1H), 7.74 (d, J=7.8 Hz, 2H), 7.51 (d, J=4.2 Hz, 2H), 7.37 (dd, J=7.5, 1.0 Hz, 1H), 7.30-7.21 (m, 1H), 7.10 (dtd, J=25.4, 7.6, 1.3 Hz, 2H), 6.15 (s, 1H), 2.27 (s, 3H).

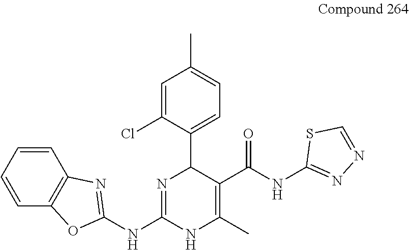 [SMILES: CC1=C(C(=O)Nc2nncs2)C(c2ccc(C)cc2Cl)N=C(Nc2nc3ccccc3o2)N1]

The title compound 2-(benzo[d]oxazol-2-ylamino)-4-(2-chloro-4-methylphenyl)-6-methyl-N-(1,3,4-thiadiazol-2-yl)-1,4-dihydropyrimidine-5-carboxamide was prepared according to Example 7 using ethyl 3-oxobutanoate in Step 1 and using 2-chloro-4-methylbenzaldehyde in Step 2 as starting material. MS m/z (M+H⁺) 480.1, ¹H NMR (400 MHz, DMSO-d₆) δ 13.04 (s, 1H), 12.45 (s, 1H), 10.36-10.28 (m, 2H), 9.10 (s, 1H), 7.58-7.50 (m, 1H), 7.42-7.22 (m, 3H), 7.25-7.04 (m, 2H), 6.16 (d, J=2.8 Hz, 1H), 2.28 (s, 3H), 2.21 (s, 3H).

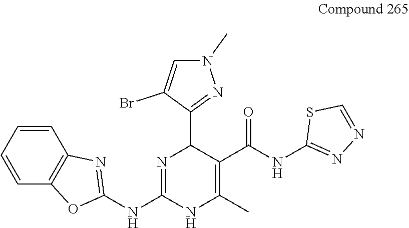 [SMILES: CC1=C(C(=O)Nc2nncs2)C(c2nn(C)cc2Br)N=C(Nc2nc3ccccc3o2)N1]

The title compound 2-(benzo[d]oxazol-2-ylamino)-4-(4-bromo-1-methyl-1H-pyrazol-3-yl)-6-methyl-N-(1,3,4-thiadiazol-2-yl)-1,4-dihydropyrimidine-5-carboxamide was prepared according to Example 7 using ethyl 3-oxobutanoate in Step 1 and using 4-bromo-1-methyl-1H-pyrazole-3-carbaldehyde in Step 2 as starting material. MS m/z (M+H⁺) 514, ¹H NMR (400 MHz, DMSO-d₆) δ 12.24 (s, 1H), 10.30 (s, 1H), 9.99 (s, 1H), 9.10 (s, 1H), 7.58-7.49 (m, 1H), 7.43-7.33 (m, 2H), 7.31-7.19 (m, 1H), 7.13 (dtd, J=26.9, 7.6, 1.3 Hz, 1H), 6.07 (d, J=3.0 Hz, 1H), 3.71 (s, 3H), 2.19 (s, 3H).

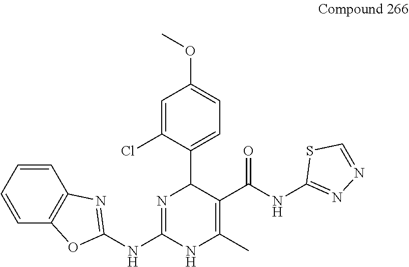 [SMILES: COc1ccc(C2N=C(Nc3nc4ccccc4o3)NC(C)=C2C(=O)Nc2nncs2)c(Cl)c1]

The title compound 2-(benzo[d]oxazol-2-ylamino)-4-(2-chloro-4-methoxyphenyl)-6-methyl-N-(1,3,4-thiadiazol-2-yl)-1,4-dihydropyrimidine-5-carboxamide was prepared according to Example 7 using ethyl 3-oxobutanoate in Step 1 and using 2-chloro-4-methoxybenzaldehyde in Step 2 as starting material. MS m/z (M+H⁺) 496.1, ¹H NMR (400 MHz, DMSO-d₆) δ 13.02 (s, 1H), 12.44 (s, 1H), 10.30 (s, 2H), 9.13 (s, 1H), 7.54 (ddd, J=7.5, 4.1, 1.3 Hz, 1H), 7.41-7.33 (m, 1H), 7.33-7.12 (m, 1H), 7.12-7.03 (m, 1H), 6.95 (dd, J=8.6, 2.5 Hz, 1H), 6.89 (dd, J=8.7, 2.6 Hz, 1H), 6.14 (d, J=2.7 Hz, 1H), 3.70 (s, 3H), 2.27 (s, 3H).

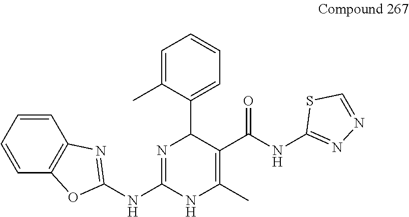 [SMILES: CC1=C(C(=O)Nc2nncs2)C(c2ccccc2C)N=C(Nc2nc3ccccc3o2)N1]

The title compound 2-(benzo[d]oxazol-2-ylamino)-6-methyl-N-(1,3,4-thiadiazol-2-yl)-4-(o-tolyl)-1,4-dihydropyrimidine-5-carboxamide was prepared according to Example 7 using ethyl 3-oxobutanoate in Step 1 and using 2-methylbenzaldehyde in Step 2 as starting material. MS m/z (M+H⁺) 446.1, ¹H NMR (400 MHz, DMSO-d₆) δ 13.00 (s, 1H), 12.36 (s, 1H), 10.28 (d, J=1.8 Hz, 1H), 9.97 (t, J=2.3 Hz, 1H), 9.11 (d, J=26.9 Hz, 1H), 7.58-7.48 (m, 1H), 7.41-7.32 (m, 2H), 7.32-7.22 (m, 2H), 7.15 (ddt, J=7.6, 6.5, 1.4 Hz, 1H), 7.11-7.04 (m, 1H), 6.11 (dd, J=2.8, 1.1 Hz, 1H), 2.51 (s, 3H), 2.25 (s, 3H).

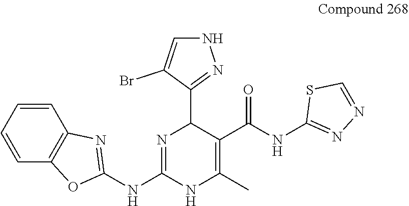 [SMILES: CC1=C(C(=O)Nc2nncs2)C(c2n[nH]cc2Br)N=C(Nc2nc3ccccc3o2)N1]

The title compound 2-(benzo[d]oxazol-2-ylamino)-4-(4-bromo-1H-pyrazol-3-yl)-6-methyl-N-(1,3,4-thiadiazol-2-yl)-1,4-dihydropyrimidine-5-carboxamide was prepared according to Example 7 using ethyl 3-oxobutanoate in Step 1 and using 4-bromo-1H-pyrazole-3-carbaldehyde in Step 2 as starting material. MS m/z (M+H⁺) 502, ¹H NMR (400 MHz, DMSO-d₆) δ 12.94 (s, 1H), 12.22 (s, 1H), 10.02 (s, 1H), 7.59-7.48 (m, 1H), 7.45-7.34 (m, 2H), 7.31-7.18 (m, 2H), 7.12 (dtd, J=26.4, 7.6, 1.3 Hz, 2H), 6.12 (s, 1H), 2.22 (s, 3H).

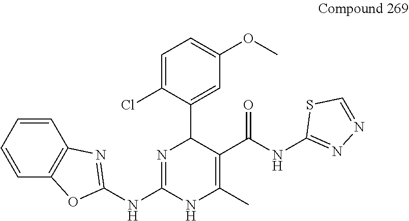 [SMILES: COc1ccc(Cl)c(C2N=C(Nc3nc4ccccc4o3)NC(C)=C2C(=O)Nc2nncs2)c1]

The title compound 2-(benzo[d]oxazol-2-ylamino)-4-(2-chloro-5-methoxyphenyl)-6-methyl-N-(1,3,4-thiadiazol-2-yl)-1,4-dihydropyrimidine-5-carboxamide was prepared according to Example 7 using ethyl 3-oxobutanoate in Step 1 and using 2-chloro-5-methoxybenzaldehyde in Step 2 as starting material. MS m/z (M+H⁺) 496.1.

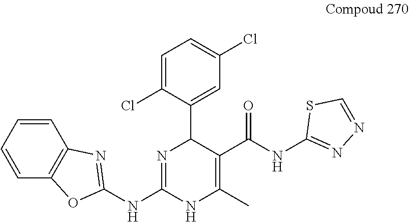 [SMILES: CC1=C(C(=O)Nc2nncs2)C(c2cc(Cl)ccc2Cl)N=C(Nc2nc3ccccc3o2)N1]

The title compound 2-(benzo[d]oxazol-2-ylamino)-4-(2,5-dichlorophenyl)-6-methyl-N-(1,3,4-thiadiazol-2-yl)-1,4-dihydropyrimidine-5-carboxamide was prepared according to Example 7 using ethyl 3-oxobutanoate in Step 1 and using 2,5-dichlorobenzaldehyde in Step 2 as starting material. MS m/z (M+H⁺) 500, ¹H NMR (400 MHz, DMSO-d₆) δ 12.54 (s, 1H), 10.38 (s, 2H), 9.15 (d, J=21.4 Hz, 1H), 7.53 (d, J=8.5 Hz, 2H), 7.45-7.34 (m, 3H), 7.13 (dtd, J=28.0, 7.6, 1.3 Hz, 2H), 6.13 (d, J=2.9 Hz, 1H), 2.29 (d, J=4.8 Hz, 3H).

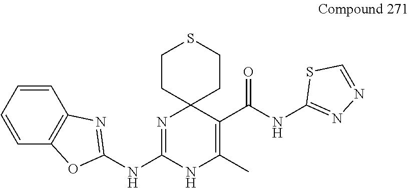 [SMILES: CC1=C(C(=O)Nc2nncs2)C2(CCSCC2)N=C(Nc2nc3ccccc3o2)N1]

The title compound 2-(benzo[d]oxazol-2-ylamino)-4-methyl-N-(1,3,4-thiadiazol-2-yl)-9-thia-1,3-diazaspiro[5.5]undeca-1,4-diene-5-carboxamide was prepared according to Example 7 using ethyl 3-oxobutanoate in Step 1 and using dihydro-2H-thiopyran-4(3H)-one in Step 2 as starting material. MS m/z (M+H⁺) 442.1, ¹H NMR (400 MHz, DMSO-d₆) δ 12.71 (s, 1H), 10.32 (s, 1H), 10.11 (s, 1H), 9.19 (s, 1H), 7.51-7.28 (m, 2H), 7.27-7.00 (m, 2H), 2.96-2.83 (m, 2H), 2.56 (d, J=14.4 Hz, 2H), 2.16-2.04 (m, 4H), 1.97-1.84 (m, 3H).

### Example 8

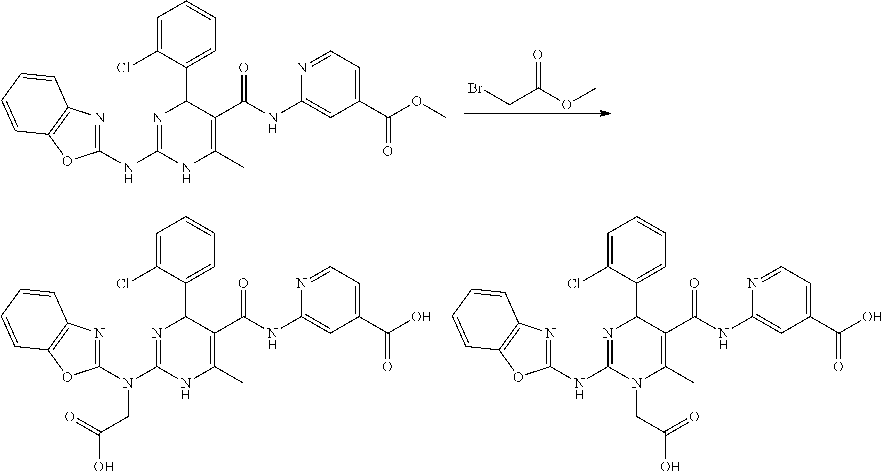

A mixture of methyl 2-(2-(benzo[d]oxazol-2-ylamino)-4-(2-chlorophenyl)-6-methyl-1,4-dihydropyrimidine-5-carboxamido)isonicotinate (40 mg, 0.077 mmol) and K₂CO₃ (53.5 mg, 0.387 mmol) in DMF (774 μL) was stirred at 23° C. Then, methyl 2-bromoacetate (23.67 mg, 0.155 mmol) was added and stirred for 16 h at 70° C. Then, NaOH, EtOH, and water were added to hydrolyze. The crude product was purified. MS m/z (M+H⁺) 561.1. (Compound 272)

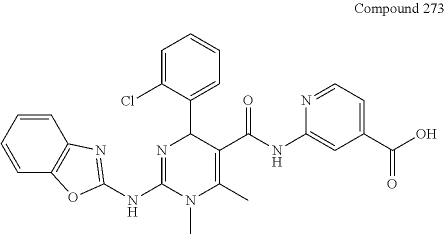 [SMILES: CC1=C(C(=O)Nc2cc(C(=O)O)ccn2)C(c2ccccc2Cl)N=C(Nc2nc3ccccc3o2)N1C]

The title compound 2-(2-(benzo[d]oxazol-2-ylamino)-4-(2-chlorophenyl)-1,6-dimethyl-1,4-dihydropyrimidine-5-carboxamido)isonicotinic acid was prepared according to Example 8 using methyl iodide as starting material. MS m/z (M+H⁺) 517.1.

### Example 9

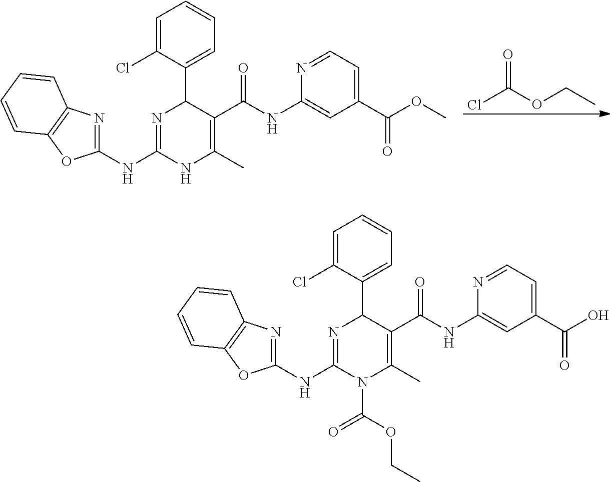

A mixture of methyl 2-(2-(benzo[d]oxazol-2-ylamino)-4-(2-chlorophenyl)-6-methyl-1,4-dihydropyrimidine-5-carboxamido)isonicotinate (50 mg, 0.097 mmol) and Hunig's Base (84 μL, 0.484 mmol) in THF (967 μL) was treated with ethyl carbonochloridate (26.2 mg, 0.242 mmol) and stirred for 4 h at 70° C. Complete conversion. This mixture was diluted with EtOH (1 mL) and water (1 mL) and then treated with sodium hydroxide (38.7 mg, 0.967 mmol). It was stirred for 10 minutes at RT and quenched with acetic acid (83 μL, 1.451 mmol). It was diluted with DMSO and purified. MS m/z (M+H⁺) 575.1. (Compound 274)

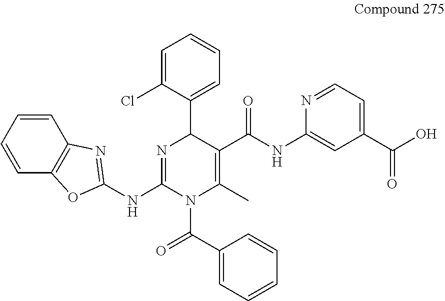 [SMILES: CC1=C(C(=O)Nc2cc(C(=O)O)ccn2)C(c2ccccc2Cl)N=C(Nc2nc3ccccc3o2)N1C(=O)c1ccccc1]

The title compound 2-(2-(benzo[d]oxazol-2-ylamino)-1-benzoyl-4-(2-chlorophenyl)-6-methyl-1,4-dihydropyrimidine-5-carboxamido)isonicotinic acid was prepared according to Example 9 using benzoyl chloride as starting material. MS m/z (M+H⁺) 607.2.

### Example 10

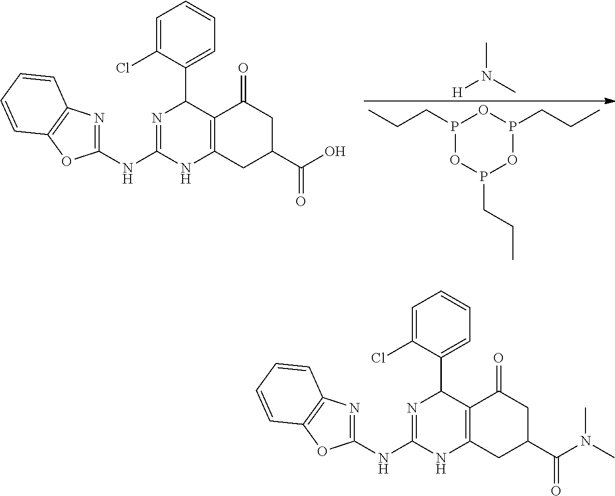

To 2-(benzo[d]oxazol-2-ylamino)-4-(2-chlorophenyl)-5-oxo-1,4,5,6,7,8-hexahydroquinazoline-7-carboxylic acid (20 mg, 0.046 mmol) in DMF (916 μL) was added dimethylamine (45.8 μL, 0.092 mmol) and 2,4,6-tripropyl-1,3,5,2,4,6-trioxatriphosphinane (24.74 μL, 0.046 mmol). This reaction was stirred at RT for 1 h. Reaction is dilute with DMSO and submit for purification. MS m/z (M+H⁺) 463.8. (Compound 277)

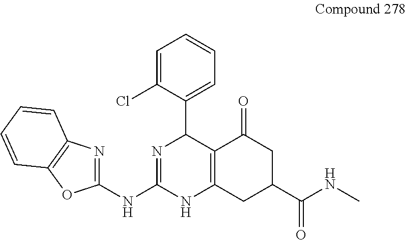 [SMILES: CNC(=O)C1CC(=O)C2=C(C1)NC(Nc1nc3ccccc3o1)=NC2c1ccccc1Cl]

The title compound 2-(benzo[d]oxazol-2-ylamino)-4-(2-chlorophenyl)-N-methyl-5-oxo-1,4,5,6,7,8-hexahydroquinazoline-7-carboxamide was prepared according to Example 10 using methanamine hydrochloride as starting material. MS m/z (M+H⁺) 449.8.

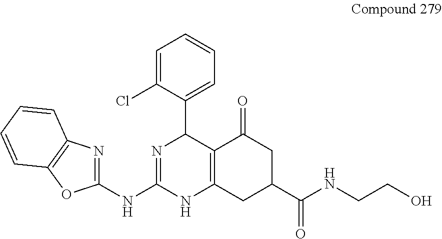 [SMILES: O=C1CC(C(=O)NCCO)CC2=C1C(c1ccccc1Cl)N=C(Nc1nc3ccccc3o1)N2]

The title compound 2-(benzo[d]oxazol-2-ylamino)-4-(2-chlorophenyl)-N-(2-hydroxyethyl)-5-oxo-1,4,5,6,7,8-hexahydroquinazoline-7-carboxamide was prepared according to Example 10 using 2-aminoethanol as starting material. MS m/z (M+H⁺) 479.8.

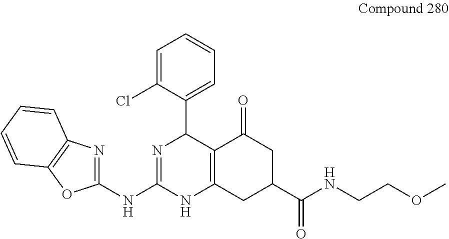 [SMILES: COCCNC(=O)C1CC(=O)C2=C(C1)NC(Nc1nc3ccccc3o1)=NC2c1ccccc1Cl]

The title compound 2-(benzo[d]oxazol-2-ylamino)-4-(2-chlorophenyl)-N-(2-methoxyethyl)-5-oxo-1,4,5,6,7,8-hexahydroquinazoline-7-carboxamide was prepared according to Example 10 using 2-methoxyethanamine as starting material. MS m/z (M+H⁺) 493.8.

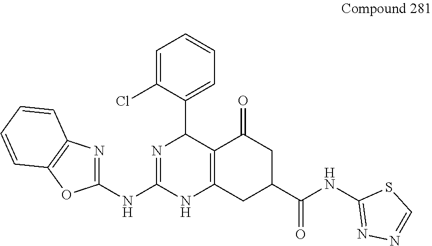 [SMILES: O=C1CC(C(=O)Nc2nncs2)CC2=C1C(c1ccccc1Cl)N=C(Nc1nc3ccccc3o1)N2]

The title compound 2-(benzo[d]oxazol-2-ylamino)-4-(2-chlorophenyl)-5-oxo-N-(1,3,4-thiadiazol-2-yl)-1,4,5,6,7,8-hexahydroquinazoline-7-carboxamide was prepared according to Example 10 using 1,3,4-thiadiazol-2-amine as starting material. MS m/z (M+H⁺) 519.7.

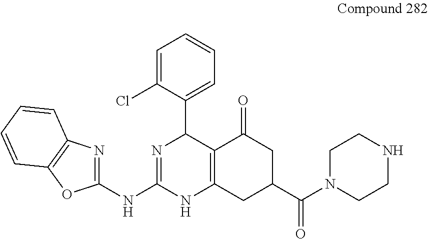 [SMILES: O=C1CC(C(=O)N2CCNCC2)CC2=C1C(c1ccccc1Cl)N=C(Nc1nc3ccccc3o1)N2]

The title compound 2-(benzo[d]oxazol-2-ylamino)-4-(2-chlorophenyl)-7-(piperazine-1-carbonyl)-4,6,7,8-tetrahydroquinazolin-5(1H)-one was prepared according to Example 10 using tert-butyl piperazine-1-carboxylate as starting material. MS m/z (M+H⁺) 505.1.

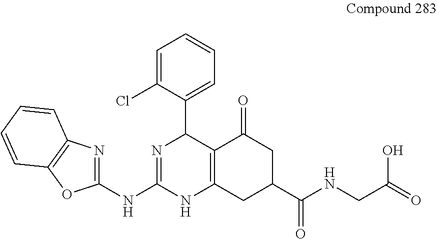 [SMILES: O=C(O)CNC(=O)C1CC(=O)C2=C(C1)NC(Nc1nc3ccccc3o1)=NC2c1ccccc1Cl]

The title compound 2-(2-(benzo[d]oxazol-2-ylamino)-4-(2-chlorophenyl)-5-oxo-1,4,5,6,7,8-hexahydroquinazoline-7-carboxamido)acetic acid was prepared according to Example 10 using tert-butyl 2-aminoacetate as starting material. MS m/z (M+H⁺) 494.1.

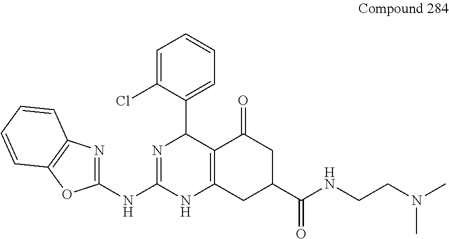 [SMILES: CN(C)CCNC(=O)C1CC(=O)C2=C(C1)NC(Nc1nc3ccccc3o1)=NC2c1ccccc1Cl]

The title compound 2-(benzo[d]oxazol-2-ylamino)-4-(2-chlorophenyl)-N-(2-(dimethylamino)ethyl)-5-oxo-1,4,5,6,7,8-hexahydroquinazoline-7-carboxamide was prepared according to Example 10 using N1,N1-dimethylethane-1,2-diamine as starting material. MS m/z (M+H⁺) 507.1.

### Example 11

### SAR Analysis and Results

Structure-activity relationships (SAR) studies were conducted using traditional SAR campaigns.

### Biochemical and Cellular Inhibitory Analyses

In vitro hGALK1 activity assays were performed as previously described. Liu et al., Bioorg. Med. Chem. Lett. 25(3): 721-727 (2015), which is incorporated by reference herein for the specific teachings thereof. The biochemical assay was performed by coupling the activity of recombinant human or mouse GALK1 to the Kinase-Glo Plus luminescent ATP detection kit, using ATP depletion as a measure of GALK1 turnover. Specifically, three μL/well of ATP substrate solution (35 μM ATP) in assay buffer (20 mM HEPES pH 8.0, 5 mM MgCl₂, 60 mM NaCl, 1 mM DTT, 0.01% BSA final concentration) was dispensed into 1536-well assay plates (Greiner, white solid-bottom medium-binding plates). Aliquots of compound (23 nL solubilized in DMSO) were transferred to the assay plates using a Kalypsys 1536-well pintool such that 11 concentrations with 1/3 dilutions ranging from 57.4 μM-0.97 nM are tested. One μL/well of GALK-galactose solution (5 nM GALK1, 100 μM galactose) in assay buffer was then added, yielding a final reaction volume of 4 μL/well. Following a 1-hour room temperature incubation, 4 μL of Kinase-Glo Plus detection reagent was added to provide an ATP-dependent luminescent readout (final assay volume: 8 μL/well). Luminescence was detected using a ViewLux plate reader (PerkinElmer) after a 10-minute incubation, using a 1 second exposure time and 2× binning.

### Gal-1-p Accumulation Assay

Skin fibroblasts derived from GALT-deficient patients were maintained in galactose-free culture medium supplemented with 10% hexose-free fetal bovine serum (FBS). Before galactose challenge, inhibitors were added to the medium at designated concentrations and incubated at 37° C. for 4 hr. Then, galactose was added to reach 0.05% in the medium. After 4 hr of challenge, cells were collected and washed twice with PBS. Then, the cells were disrupted in 300 μL of ice-cold hypotonic buffer containing 25 mM Tris-HCl (pH 7.4), 25 mM NaCl, 0.5 mM EDTA, and protease inhibitor cocktail (Roche). The lysates were passed five times through a 30-gauge needle and centrifuged for 20 min at 16,000×g and 4° C. A small portion of supernatant was saved for protein concentration measurement. Gal-1-p level was measured using the alkaline phosphatase coupled method previously described. The gal-1-p concentration was normalized to protein concentration. The assay was analyzed using the paired t-test to determine the statistical difference between the compound treated cells and corresponding DMSO control. The two-sided p value less than 0.05 was considered statistically significant.

For measurement of cellular Gal-1P reduction, compounds at different concentrations were added to cultured patient fibroblast at 80% confluency. After incubating the compounds for 16 hours, 10 mM galactose was added to the culture. After another 4-hours, cells were washed with cold PBS buffer 3× and harvested for gal-1P measurement, according to previously published method. Liu et al., Bioorg. Med. Chem. Lett. 25(3): 721-727 (2015).

TABLE 1
SAR Results for Compounds 1-284

| | | hGAL | mGAL | Metab. | Perm. | Sol. |
|---|---|---|---|---|---|---|
| Cpd. | | K | K | Stabil. | (1 × 10⁻⁶ | (μg/ |
| No. | Structure and Name | (μM) | (μM) | (m) | cm/sec) | mL) |
| 1 | 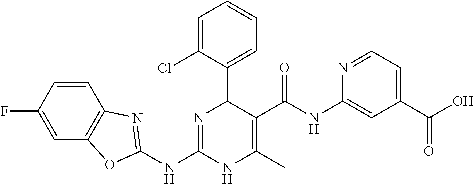 [SMILES: CC1=C(C(=O)Nc2cc(C(=O)O)ccn2)C(c2ccccc2Cl)N=C(Nc2nc3ccc(F)cc3o2)N1] | 0.68 | 4.32 | >30 | <1 | 46.1 |
| | 2-(4-(2-chlorophenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 2 | 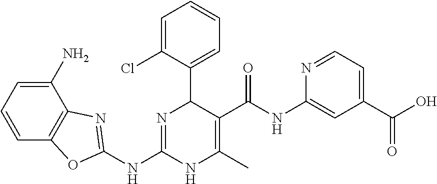 [SMILES: CC1=C(C(=O)Nc2cc(C(=O)O)ccn2)C(c2ccccc2Cl)N=C(Nc2nc3c(N)cccc3o2)N1] | 21.67 (−1.2) | 30.61 (−1.2) | >30 | <1 | >76 |
| | 2-(2-((4-aminobenzo[d]oxazol-2-yl)amino)-4-(2- | | | | | |
| | chlorophenyl)-6-methyl-1,4-dihydropyrimidine- | | | | | |
| | 5-carboxamido)isonicotinic acid | | | | | |
| 3 | 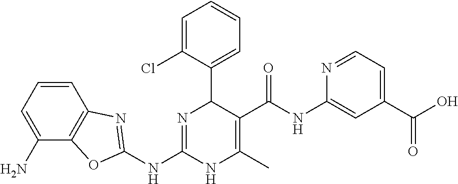 [SMILES: CC1=C(C(=O)Nc2cc(C(=O)O)ccn2)C(c2ccccc2Cl)N=C(Nc2nc3cccc(N)c3o2)N1] | 0.22 | 3.43 | >30 | <1 | >76 |
| | 2(2-((7-aminobenzo[d]oxazol-2-yl)amino)-4-(2- | | | | | |
| | chlorophenyl)-6-methyl-1,4-dihydropyrimidine- | | | | | |
| | 5-carboxamido)isonicotinic acid | | | | | |
| 4 | 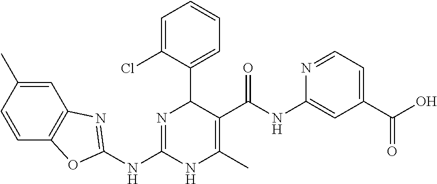 [SMILES: CC1=C(C(=O)Nc2cc(C(=O)O)ccn2)C(c2ccccc2Cl)N=C(Nc2nc3cc(C)ccc3o2)N1] | 1.37 | 3.06 | >30 | <1 | <1 |
| | 2-(4-(2-chlorophenyl)-6-methyl-2-((5- | | | | | |
| | methylbenzo[d]oxazol-2-yl)amino)-1,4- | | | | | |
| | dihydropyrimidine-5-carboxamido)isonicotinic | | | | | |
| | acid | | | | | |
| 5 | 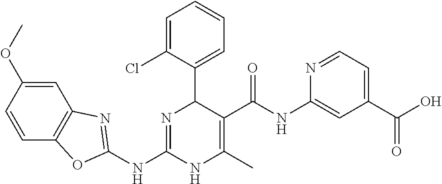 [SMILES: COc1ccc2oc(NC3=NC(c4ccccc4Cl)C(C(=O)Nc4cc(C(=O)O)ccn4)=C(C)N3)nc2c1] | 6.85 | 12.19 (−1.2) | >30 | <1 | >79 |
| | 2-(4-(2-chlorophenyl)-2-((5- | | | | | |
| | methoxybenzo[d]oxazol-2-yl)amino)-6-methyl- | | | | | |
| | 1,4-dihydropyrimidine-5- | | | | | |
| | carboxamido)isonicotinic acid | | | | | |
| 6 | 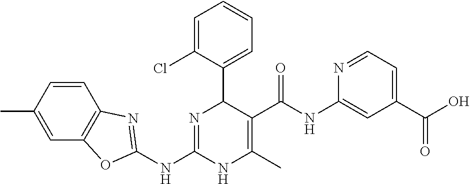 [SMILES: CC1=C(C(=O)Nc2cc(C(=O)O)ccn2)C(c2ccccc2Cl)N=C(Nc2nc3ccc(C)cc3o2)N1] | 5.44 | 17.21 (−1.2) | >30 | <1 | 34.42 |
| | 2-(4-(2-chlorophenyl)-6-methyl-2-((6- | | | | | |
| | methylbenzo[d]oxazol-2-yl)amino)-1,4- | | | | | |
| | dihydropyrimidine-5-carboxamido)isonicotinic | | | | | |
| | acid | | | | | |
| 7 | 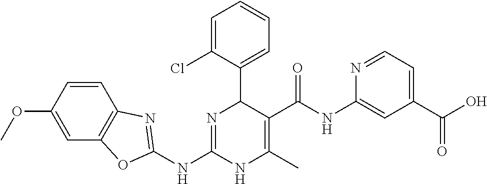 [SMILES: COc1ccc2nc(NC3=NC(c4ccccc4Cl)C(C(=O)Nc4cc(C(=O)O)ccn4)=C(C)N3)oc2c1] | 21.67 (−1.2) | 21.67 (−1.2) | >30 | <1 | >79 |
| | 2-(4-(2-chlorophenyl)-6-methyl-2-((6- | | | | | |
| | methoxybenzo[d]oxazol-2-yl)amino)-1,4- | | | | | |
| | dihydropyrimidine-5-carboxamido)isonicotinic | | | | | |
| | acid | | | | | |
| 8 | 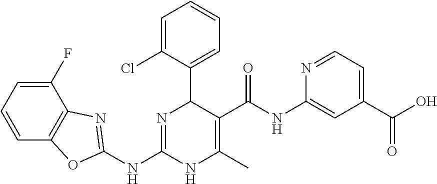 [SMILES: CC1=C(C(=O)Nc2cc(C(=O)O)ccn2)C(c2ccccc2Cl)N=C(Nc2nc3c(F)cccc3o2)N1] | 3.85 | 21.67 (−1.2) | na | na | na |
| | 2-(4-(2-chlorophenyl)-6-methyl-2-((4- | | | | | |
| | fluorobenzo[d]oxazol-2-yl)amino)-1,4- | | | | | |
| | dihydropyrimidine-5-carboxamido)isonicotinic | | | | | |
| | acid | | | | | |
| 9 | 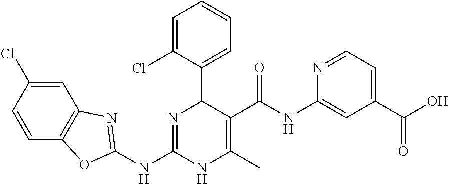 [SMILES: CC1=C(C(=O)Nc2cc(C(=O)O)ccn2)C(c2ccccc2Cl)N=C(Nc2nc3cc(Cl)ccc3o2)N1] | 2.43 | 6.11 | >30 | <1 | 35.2 |
| | 2-(2-((5-chlorobenzo[d]oxazol-2-yl)amino)-4-(2- | | | | | |
| | chlorophenyl)-6-methyl-1,4-dihydropyrimidine- | | | | | |
| | 5-carboxamido)isonicotinic acid | | | | | |
| 10 | 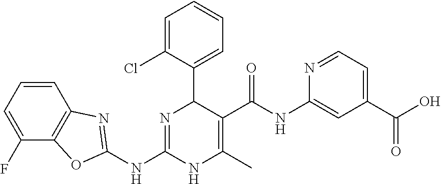 [SMILES: CC1=C(C(=O)Nc2cc(C(=O)O)ccn2)C(c2ccccc2Cl)N=C(Nc2nc3cccc(F)c3o2)N1] | 1.72 | 8.63 | >30 | <1 | >77 |
| | 2-(4-(2-chlorophenyl)-2-((7- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 11 | 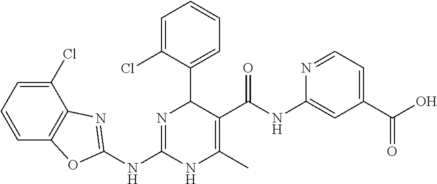 [SMILES: CC1=C(C(=O)Nc2cc(C(=O)O)ccn2)C(c2ccccc2Cl)N=C(Nc2nc3c(Cl)cccc3o2)N1] | 5.44 | 13.67 (−1.2) | >30 | <1.3 | <1 |
| | 2-(2-((4-chlorobenzo[d]oxazol-2-yl)amino)-4-(2- | | | | | |
| | chlorophenyl)-6-methyl-1,4-dihydropyrimidine- | | | | | |
| | 5-carboxamido)isonicotinic acid | | | | | |
| 12 | 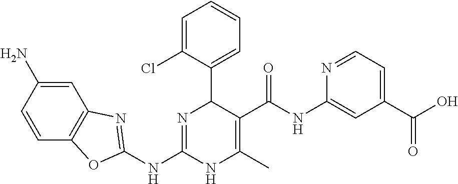 [SMILES: CC1=C(C(=O)Nc2cc(C(=O)O)ccn2)C(c2ccccc2Cl)N=C(Nc2nc3cc(N)ccc3o2)N1] | 7.69 | 21.67 (−1.2) | >30 | na | >76 |
| | 2-(2-((5-aminobenzo[d]oxazol-2-yl)amino)-4-(2- | | | | | |
| | chlorophenyl)-6-methyl-1,4-dihydropyrimidine- | | | | | |
| | 5-carboxamido)isonicotinic acid | | | | | |
| 13 | 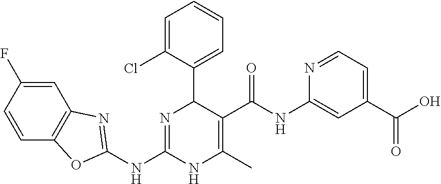 [SMILES: CC1=C(C(=O)Nc2cc(C(=O)O)ccn2)C(c2ccccc2Cl)N=C(Nc2nc3cc(F)ccc3o2)N1] | 3.43 | 17.21 | na | na | na |
| | 2-(4-(2-chlorophenyl)-2-((5- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 14 | 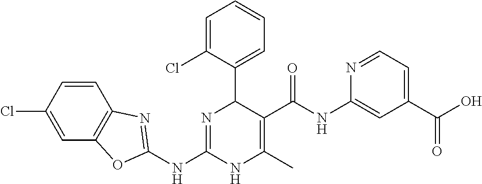 [SMILES: CC1=C(C(=O)Nc2cc(C(=O)O)ccn2)C(c2ccccc2Cl)N=C(Nc2nc3ccc(Cl)cc3o2)N1] | 1.93 | 6.11 | >30 | Na | 6.3 |
| | 2-(2-((6-chlorobenzo[d]oxazol-2-yl)amino)-4-(2- | | | | | |
| | chlorophenyl)-6-methyl-1,4-dihydropyrimidine- | | | | | |
| | 5-carboxamido)isonicotinic acid | | | | | |
| 15 | 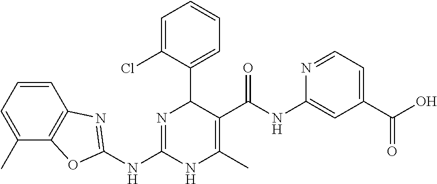 [SMILES: CC1=C(C(=O)Nc2cc(C(=O)O)ccn2)C(c2ccccc2Cl)N=C(Nc2nc3cccc(C)c3o2)N1] | 0.97 | 6.85 | >30 | na | >76 |
| | 2-(4-(2-chlorophenyl)-6-methyl-2-((7- | | | | | |
| | methylbenzo[d]oxazol-2-yl)amino)-1,4- | | | | | |
| | dihydropyrimidine-5-carboxamido)isonicotinic acid | | | | | |
| 16 | 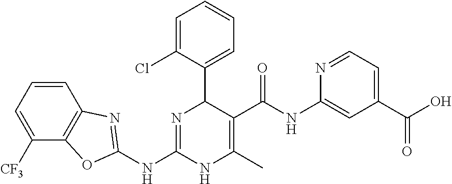 [SMILES: CC1=C(C(=O)Nc2cc(C(=O)O)ccn2)C(c2ccccc2Cl)N=C(Nc2nc3cccc(C(F)(F)F)c3o2)N1] | 21.67 (−1.2) | 48.62 (−2.2) | >30 | <1 | 61.135 |
| | 2-(4-(2-chlorophenyl)-6-methyl-2-((7- | | | | | |
| | (trifluoromethyl)benzo[d]oxazol-2-yl)amino)- | | | | | |
| | 1,4-dihydropyrimidine-5- | | | | | |
| | carboxamido)isonicotinic acid | | | | | |
| 17 | 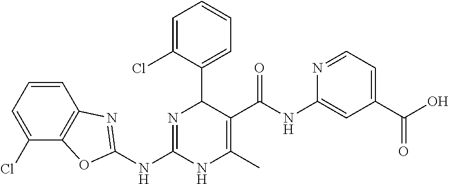 [SMILES: CC1=C(C(=O)Nc2cc(C(=O)O)ccn2)C(c2ccccc2Cl)N=C(Nc2nc3cccc(Cl)c3o2)N1] | 1.93 | 9.68 | >30 | <1 | 57.5 |
| | 2-(2-((7-chlorobenzo[d]oxazol-2-yl)amino)-4-(2- | | | | | |
| | chlorophenyl)-6-methyl-1,4-dihydropyrimidine- | | | | | |
| | 5-carboxamido)isonicotinic acid | | | | | |
| 18 | 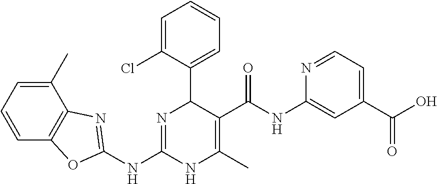 [SMILES: CC1=C(C(=O)Nc2cc(C(=O)O)ccn2)C(c2ccccc2Cl)N=C(Nc2nc3c(C)cccc3o2)N1] | 9.68 | 17.21 (−1.2) | >30 | <1.3 | 3.5 |
| | 2-(4-(2-chlorophenyl)-6-methyl-2-((4- | | | | | |
| | methylbenzo[d]oxazol-2-yl)amino)-1,4- | | | | | |
| | dihydropyrimidine-5-carboxamido)isonicotinic | | | | | |
| | acid | | | | | |
| 19 | 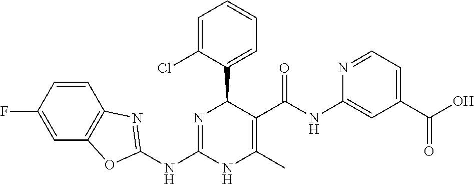 [SMILES: CC1=C(C(=O)Nc2cc(C(=O)O)ccn2)[C@H](c2ccccc2Cl)N=C(Nc2nc3ccc(F)cc3o2)N1] | 0.24 | 1.72 | >30 | <1 | 55.6 |
| | (R)-2-(4-(2-chlorophenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 20 | 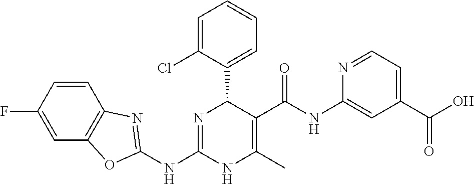 [SMILES: CC1=C(C(=O)Nc2cc(C(=O)O)ccn2)[C@@H](c2ccccc2Cl)N=C(Nc2nc3ccc(F)cc3o2)N1] | 3.06 | 13.67 (−1.2) | >30 | <1 | 17.3 |
| | (S)-2-(4-(2-chlorophenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 21 | 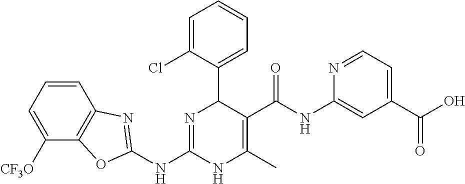 | in- active | in- active | >30 | na | 47.8 |
| | 2-(4-(2-chlorophenyl)-6-methyl-2-((7- | | | | | |
| | (trifluoromethoxy)benzo[d]oxazol-2-yl)amino)- | | | | | |
| | 1,4-dihydropyrimidine-5- | | | | | |
| | carboxamido)isonicotinic acid | | | | | |
| 22 | 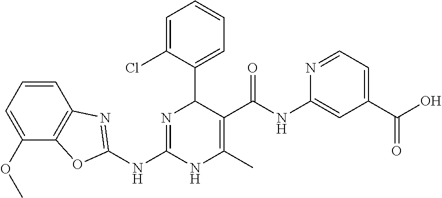 [SMILES: COc1cccc2nc(NC3=NC(c4ccccc4Cl)C(C(=O)Nc4cc(C(=O)O)ccn4)=C(C)N3)oc12] | 21.67 (−1.2) | in- active | >30 | <1 | 65.7 |
| | 2-(4-(2-chlorophenyl)-2-((7- | | | | | |
| | methoxybenzo[d]oxazol-2-yl)amino)-6-methyl- | | | | | |
| | 1,4-dihydropyrimidine-5- | | | | | |
| | carboxamido)isonicotinic acid | | | | | |
| 23 | 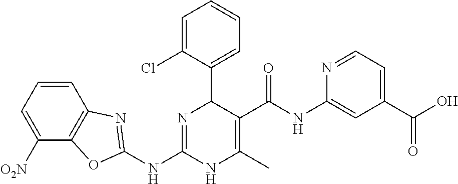 [SMILES: CC1=C(C(=O)Nc2cc(C(=O)O)ccn2)C(c2ccccc2Cl)N=C(Nc2nc3cccc([N+](=O)[O-])c3o2)N1] | 19.31 (−1.2) | 38.54 (−1.4) | >30 | 31.0 | 7.3 |
| | 2-(4-(2-chlorophenyl)-6-methyl-2-((7- | | | | | |
| | nitrobenzo[d]oxazol-2-yl)amino)-1,4- | | | | | |
| | dihydropyrimidine-5-carboxamido)isonicotinic | | | | | |
| | acid | | | | | |
| 24 | 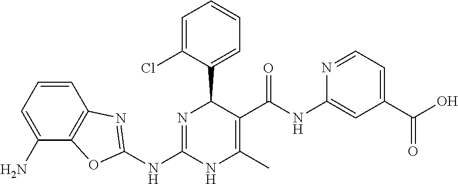 [SMILES: CC1=C(C(=O)Nc2cc(C(=O)O)ccn2)[C@H](c2ccccc2Cl)N=C(Nc2nc3cccc(N)c3o2)N1] | 0.86 | 1.72 | na | na | na |
| | (R)-2-(2-((7-aminobenzo[d]oxazol-2-yl)amino)- | | | | | |
| | 4-(2-chlorophenyl)-6-methyl-1,4- | | | | | |
| | dihydropyrimidine-5-carboxamido)isonicotinic | | | | | |
| | acid | | | | | |
| 25 | 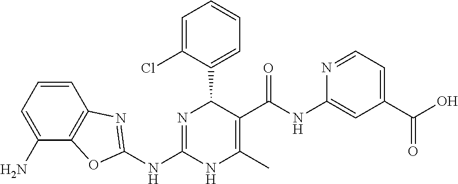 [SMILES: CC1=C(C(=O)Nc2cc(C(=O)O)ccn2)[C@@H](c2ccccc2Cl)N=C(Nc2nc3cccc(N)c3o2)N1] | 6.11 | 34.35 (−2.2) | >30 | <1.3 | >76 |
| | (S)-2-(2-((7-aminobenzo[d]oxazol-2-yl)amino)- | | | | | |
| | 4-(2-chlorophenyl)-6-methyl-1,4- | | | | | |
| | dihydropyrimidine-5-carboxamido)isonicotinic | | | | | |
| | acid | | | | | |
| 26 | 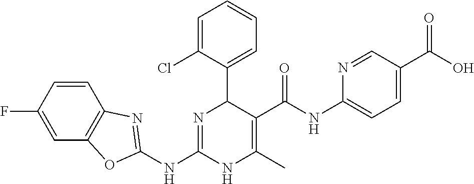 [SMILES: CC1=C(C(=O)Nc2ccc(C(=O)O)cn2)C(c2ccccc2Cl)N=C(Nc2nc3ccc(F)cc3o2)N1] | 0.68 | 3.43 | >30 | <1 | >77 |
| | 6-(4-(2-chlorophenyl)-2-((6- | | | | | |
| | fluorobenzo[d]oxazol-2-yl)amino)-6-methyl-1,4- | | | | | |
| | dihydropyrimidine-5-carboxamido)nicotinic acid | | | | | |
| 27 | 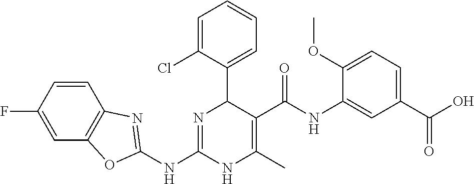 [SMILES: COc1ccc(C(=O)O)cc1NC(=O)C1=C(C)NC(Nc2nc3ccc(F)cc3o2)=NC1c1ccccc1Cl] | 2.17 | 12.19 (−1.2) | >30 | na | 53.3 |
| | 3-(4-(2-chlorophenyl)-2-((6- | | | | | |
| | fluorobenzo[d]oxazol-2-yl)amino)-6-methyl-1,4- | | | | | |
| | dihydropyrimidine-5-carboxamido)-4- | | | | | |
| | methoxybenzoic acid | | | | | |
| 28 | 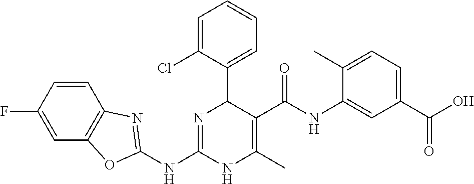 [SMILES: CC1=C(C(=O)Nc2cc(C(=O)O)ccc2C)C(c2ccccc2Cl)N=C(Nc2nc3ccc(F)cc3o2)N1] | 3.43 | 17.21 | na | na | na |
| | 3-(4-(2-chlorophenyl)-2-((6- | | | | | |
| | fluorobenzo[d]oxazol-2-yl)amino)-6-methyl-1,4- | | | | | |
| | dihydropyrimidine-5-carboxamido)-4- | | | | | |
| | methylbenzoic acid | | | | | |
| 29 | 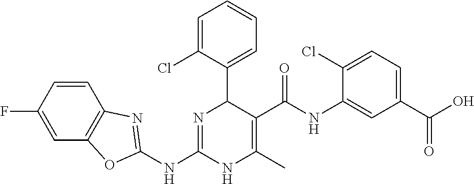 [SMILES: CC1=C(C(=O)Nc2cc(C(=O)O)ccc2Cl)C(c2ccccc2Cl)N=C(Nc2nc3ccc(F)cc3o2)N1] | 1.54 | 19.31 | >30 | 29.6 | 54.0 |
| | 4-chloro-3-(4-(2-chlorophenyl)-2-((6- | | | | | |
| | fluorobenzo[d]oxazol-2-yl)amino)-6-methyl-1,4- | | | | | |
| | dihydropyrimidine-5-carboxamido)benzoic acid | | | | | |
| 30 | 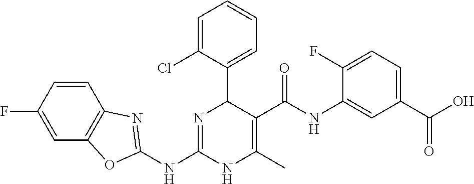 [SMILES: CC1=C(C(=O)Nc2cc(C(=O)O)ccc2F)C(c2ccccc2Cl)N=C(Nc2nc3ccc(F)cc3o2)N1] | 2.17 | 6.85 | >30 | 2.2 | 35.7 |
| | 3-(4-(2-chlorophenyl)-2-((6- | | | | | |
| | fluorobenzo[d]oxazol-2-yl)amino)-6-methyl-1,4- | | | | | |
| | dihydropyrimidine-5-carboxamido)-4- | | | | | |
| | fluorobenzoic acid | | | | | |
| 31 | 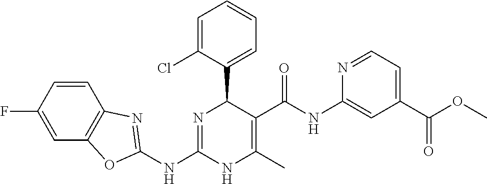 [SMILES: COC(=O)c1ccnc(NC(=O)C2=C(C)NC(Nc3nc4ccc(F)cc4o3)=N[C@H]2c2ccccc2Cl)c1] | 3.06 | 4.32 (−1.2) | 30.90853 | <21.9 | na |
| | (R)-methyl 2-(4-(2-chlorophenyl)-2-((6- | | | | | |
| | fluorobenzo[d]oxazol-2-yl)amino)-6-methyl-1,4- | | | | | |
| | dihydropyrimidine-5-carboxamido)isonicotinate | | | | | |
| 32 | 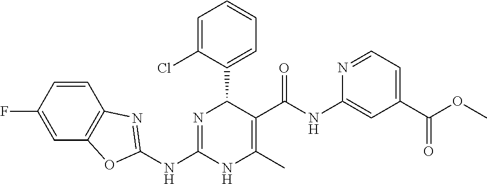 [SMILES: COC(=O)c1ccnc(NC(=O)C2=C(C)NC(Nc3nc4ccc(F)cc4o3)=N[C@@H]2c2ccccc2Cl)c1] | 12.19 (−2.2) | 86.28 (−2.4) | 24.27642 | <7.4 | na |
| | (S)-methyl 2-(4-(2-chlorophenyl)-2-((6- | | | | | |
| | fluorobenzo[d]oxazol-2-yl)amino)-6-methyl-1,4- | | | | | |
| | dihydropyrimidine-5-carboxamido)isonicotinate | | | | | |
| 33 | 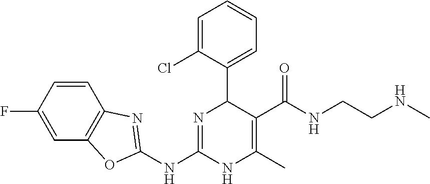 [SMILES: CNCCNC(=O)C1=C(C)NC(Nc2nc3ccc(F)cc3o2)=NC1c1ccccc1Cl] | 15.34 (−1.2) | 6.11 (−1.2) | >30 | na | >67 |
| | 4-(2-chlorophenyl)-2-((6-fluorobenzo[d]oxazol- | | | | | |
| | 2-yl)amino)-6-methyl-N-(2-(methylamino)ethyl)- | | | | | |
| | 1,4-dihydropyrimidine-5-carboxamido | | | | | |
| 34 | 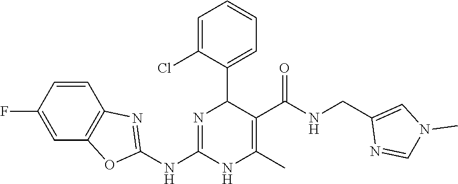 [SMILES: CC1=C(C(=O)NCc2cn(C)cn2)C(c2ccccc2Cl)N=C(Nc2nc3ccc(F)cc3o2)N1] | 8.63 | 19.31 (−1.2) | na | na | na |
| | 4-(2-chlorophenyl)-2-((6-fluorobenzo[d]oxazol- | | | | | |
| | 2-yl)amino)-6-methyl-N-((1-methyl-1H- | | | | | |
| | imidazol-4-yl)methyl)-1,4-dihydropyrimidine-5- | | | | | |
| | carboxamide | | | | | |
| 35 | 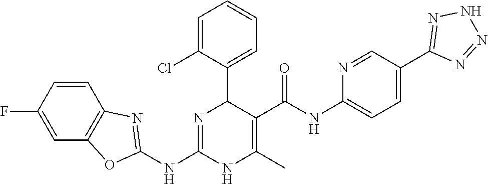 [SMILES: CC1=C(C(=O)Nc2ccc(-c3nn[nH]n3)cn2)C(c2ccccc2Cl)N=C(Nc2nc3ccc(F)cc3o2)N1] | 4.85 | 12.19 (−1.2) | na | na | na |
| | N-(5-(2H-tetrazol-5-yl)pyridin-2-yl)-4-(2- | | | | | |
| | chlorophenyl)-2-((6-fluorobenzo[d]oxazol-2- | | | | | |
| | yl)amino)-6-methyl-1,4-dihydropyrimidine-5- | | | | | |
| | carboxamide | | | | | |
| 36 | 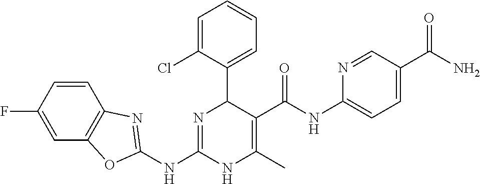 [SMILES: CC1=C(C(=O)Nc2ccc(C(N)=O)cn2)C(c2ccccc2Cl)N=C(Nc2nc3ccc(F)cc3o2)N1] | 2.43 | 6.85 | na | na | na |
| | N-(5-carbamoylpyridin-2-yl)-4-(2-chlorophenyl)- | | | | | |
| | 2-((6-fluorobenzo[d]oxazol-2-yl)amino)-6- | | | | | |
| | methyl-1,4-dihydropyrimidine-5-carboxamide | | | | | |
| 37 | 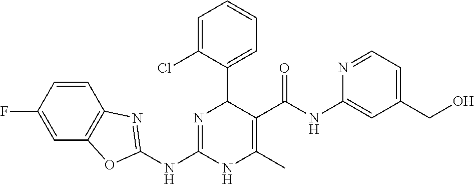 [SMILES: CC1=C(C(=O)Nc2cc(CO)ccn2)C(c2ccccc2Cl)N=C(Nc2nc3ccc(F)cc3o2)N1] | 3.06 | 10.86 | >30 | 59.9 | <1 |
| | 4-(2-chlorophenyl)-2-((6-fluorobenzo[d]oxazol- | | | | | |
| | 2-yl)amino)-N-(4-(hydroxymethyl)pyridin-2-yl)- | | | | | |
| | 6-methyl-1,4-dihydropyrimidine-5-carboxamide | | | | | |
| 38 | 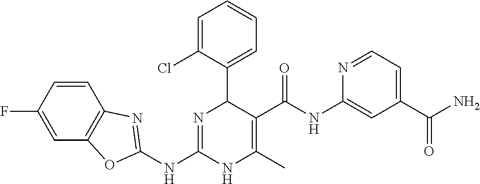 [SMILES: CC1=C(C(=O)Nc2cc(C(N)=O)ccn2)C(c2ccccc2Cl)N=C(Nc2nc3ccc(F)cc3o2)N1] | 4.85 | 19.31 (−2.1) | >30 | 15.5 | 2.4 |
| | N-(4-carbamoylpyridin-2-yl)-4-(2-chlorophenyl)- | | | | | |
| | 2-((6-fluorobenzo[d]oxazol-2-yl)amino)-6- | | | | | |
| | methyl-1,4-dihydropyrimidine-5-carboxamide | | | | | |
| 39 | 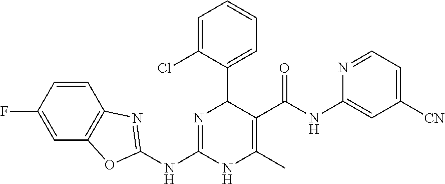 [SMILES: CC1=C(C(=O)Nc2cc(C#N)ccn2)C(c2ccccc2Cl)N=C(Nc2nc3ccc(F)cc3o2)N1] | 4.32 (−1.2) | 4.85 (−1.2) | >30 | <6.8 | <1 |
| | 4-(2-chlorophenyl)-N-(4-cyanopyridin-2-yl)-2- | | | | | |
| | ((6-fluorobenzo[d]oxazol-2-yl)amino)-6-methyl- | | | | | |
| | 1,4-dihydropyrimidine-5-carboxamide | | | | | |
| 40 | 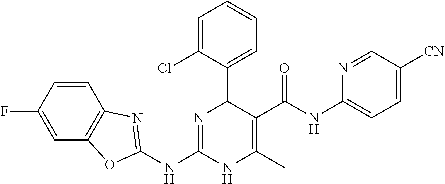 [SMILES: CC1=C(C(=O)Nc2ccc(C#N)cn2)C(c2ccccc2Cl)N=C(Nc2nc3ccc(F)cc3o2)N1] | 13.67 (−1.2) | 13.67 (−1.2) | >30 | <7.8 | <1 |
| | 4-(2-chlorophenyl)-N-(5-cyanopyridin-2-yl)-2- | | | | | |
| | ((6-fluorobenzo[d]oxazol-2-yl)amino)-6-methyl- | | | | | |
| | 1,4-dihydropyrimidine-5-carboxamide | | | | | |
| 41 | 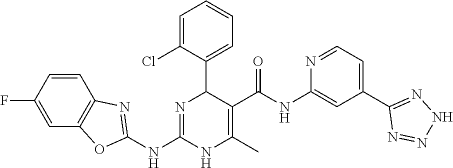 [SMILES: CC1=C(C(=O)Nc2cc(-c3nn[nH]n3)ccn2)C(c2ccccc2Cl)N=C(Nc2nc3ccc(F)cc3o2)N1] | 0.77 | 7.69 | >30 | <1 | 29.9 |
| | N-(4-(2H-tetrazol-5-yl)pyridin-2-yl)-4-(2- | | | | | |
| | chlorophenyl)-2-((6-fluorobenzo[d]oxazol-2- | | | | | |
| | yl)amino)-6-methyl-1,4-dihydropyrimidine-5- | | | | | |
| | carboxamide | | | | | |
| 42 | 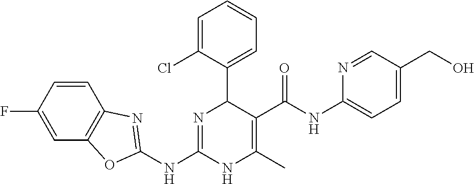 [SMILES: CC1=C(C(=O)Nc2ccc(CO)cn2)C(c2ccccc2Cl)N=C(Nc2nc3ccc(F)cc3o2)N1] | 12.19 | 19.31 (−1.2) | >30 | na | <1 |
| | 4-(2-chlorophenyl)-2-((6-fluorobenzo[d]oxazol- | | | | | |
| | 2-yl)amino)-N-(5-(hydroxymethyl)pyridin-2-yl)- | | | | | |
| | 6-methyl-1,4-dihydropyrimidine-5-carboxamide | | | | | |
| 43 | 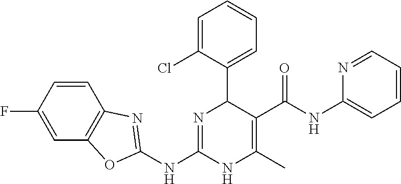 [SMILES: CC1=C(C(=O)Nc2ccccn2)C(c2ccccc2Cl)N=C(Nc2nc3ccc(F)cc3o2)N1] | 4.32 (−1.2) | 10.86 (−1.2) | >30 | 171.11 | <1 |
| | 4-(2-chlorophenyl)-2-((6-fluorobenzo[d]oxazol- | | | | | |
| | 2-yl)amino)-6-methyl-N-(pyridin-2-yl)-1,4- | | | | | |
| | dihydropyrimidine-5-carboxamide | | | | | |
| 44 | 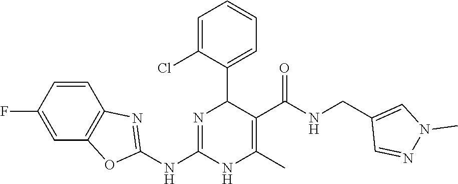 [SMILES: CC1=C(C(=O)NCc2cnn(C)c2)C(c2ccccc2Cl)N=C(Nc2nc3ccc(F)cc3o2)N1] | 1.72 | 12.19 (−1.2) | 26 | 942.87 | 13.7 |
| | 4-(2-chlorophenyl)-2-((6-fluorobenzo[d]oxazol- | | | | | |
| | 2-yl)amino)-6-methyl-N-((1-methyl-1H-pyrazol- | | | | | |
| | 4-yl)methyl)-1,4-dihydropyrimidine-5- | | | | | |
| | carboxamide | | | | | |
| 45 | 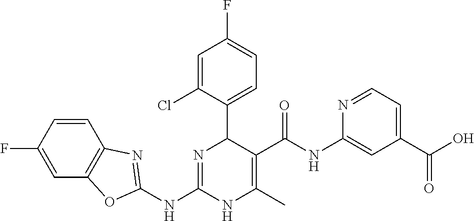 [SMILES: CC1=C(C(=O)Nc2cc(C(=O)O)ccn2)C(c2ccc(F)cc2Cl)N=C(Nc2nc3ccc(F)cc3o2)N1] | 0.86 | 4.32 | >30 | <1 | 40.6 |
| | 2-(4-(2-chloro-4-fluorophenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 46 | 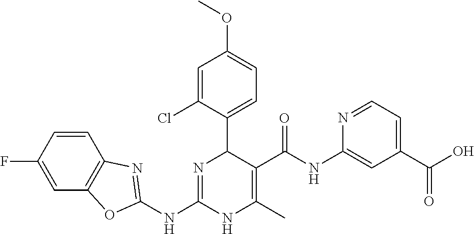 [SMILES: COc1ccc(C2N=C(Nc3nc4ccc(F)cc4o3)NC(C)=C2C(=O)Nc2cc(C(=O)O)ccn2)c(Cl)c1] | 0.43 | 3.06 | >30 | na | 8.3 |
| | 2-(4-(2-chloro-4-methoxyphenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 47 | 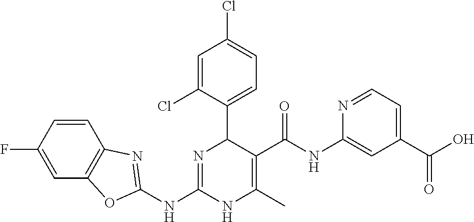 [SMILES: CC1=C(C(=O)Nc2cc(C(=O)O)ccn2)C(c2ccc(Cl)cc2Cl)N=C(Nc2nc3ccc(F)cc3o2)N1] | 1.09 | 3.85 | >30 | <1 | <1 |
| | 2-(4-(2,4-dichlorophenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 48 | 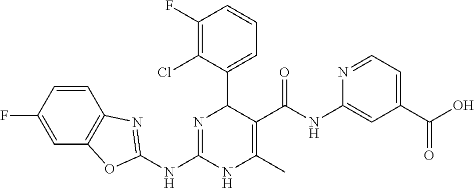 [SMILES: CC1=C(C(=O)Nc2cc(C(=O)O)ccn2)C(c2cccc(F)c2Cl)N=C(Nc2nc3ccc(F)cc3o2)N1] | 4.85 | 15.34 (1.2) | na | na | na |
| | 2-(4-(2-chloro-3-fluorophenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 49 | 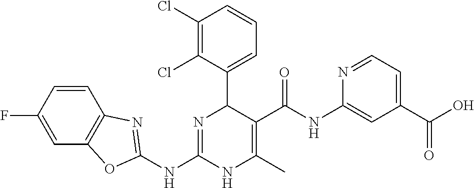 [SMILES: CC1=C(C(=O)Nc2cc(C(=O)O)ccn2)C(c2cccc(Cl)c2Cl)N=C(Nc2nc3ccc(F)cc3o2)N1] | 8.63 | 21.67 (−1.2) | >30 | na | 55.5 |
| | 2-(4-(2,3-dichlorophenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 50 | 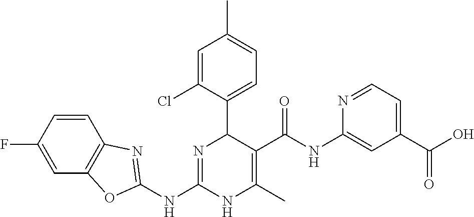 [SMILES: CC1=C(C(=O)Nc2cc(C(=O)O)ccn2)C(c2ccc(C)cc2Cl)N=C(Nc2nc3ccc(F)cc3o2)N1] | 0.39 | 3.06 | >30 | <1.5 | 2.8 |
| | 2-(4-(2-chloro-4-methylphenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 51 | 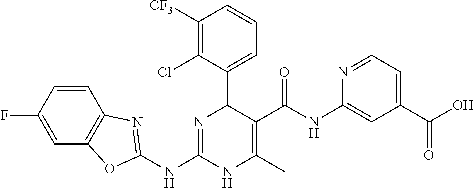 [SMILES: CC1=C(C(=O)Nc2cc(C(=O)O)ccn2)C(c2cccc(C(F)(F)F)c2Cl)N=C(Nc2nc3ccc(F)cc3o2)N1] | 19.31 (−1.2) | 27.28 (−1.2) | >30 | na | 70.1 |
| | 2-(4-(2-chloro-3-(trifluoromethyl)phenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 52 | 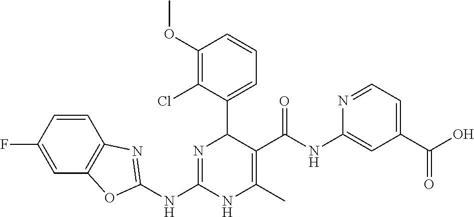 [SMILES: COc1cccc(C2N=C(Nc3nc4ccc(F)cc4o3)NC(C)=C2C(=O)Nc2cc(C(=O)O)ccn2)c1Cl] | 6.85 | 19.32 (−1.2) | >30 | <1 | >81 |
| | 2-(4-(2-chloro-3-methoxyphenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 53 | 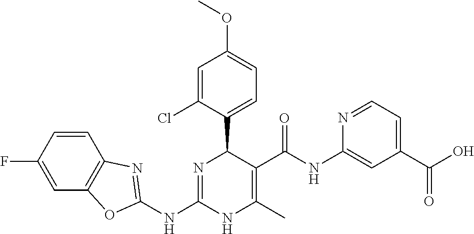 [SMILES: COc1ccc([C@@H]2N=C(Nc3nc4ccc(F)cc4o3)NC(C)=C2C(=O)Nc2cc(C(=O)O)ccn2)c(Cl)c1] | 0.25 | 1.99 | na | na | na |
| | (R)-2-(4-(2-chloro-4-methoxyphenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 54 | 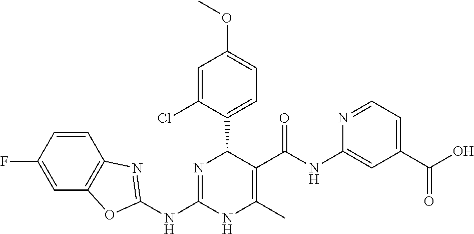 [SMILES: COc1ccc([C@H]2N=C(Nc3nc4ccc(F)cc4o3)NC(C)=C2C(=O)Nc2cc(C(=O)O)ccn2)c(Cl)c1] | 4.85 | 19.31 (−1.2) | na | na | na |
| | (S)-2-(4-(2-chloro-4-methoxyphenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 55 | 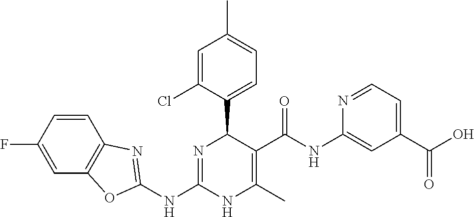 [SMILES: CC1=C(C(=O)Nc2cc(C(=O)O)ccn2)[C@H](c2ccc(C)cc2Cl)N=C(Nc2nc3ccc(F)cc3o2)N1] | 0.15 | 1.37 | >30 | <1 | >79 |
| | (R)-2-(4-(2-chloro-4-methylphenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 56 | 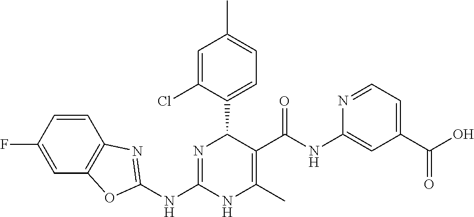 [SMILES: CC1=C(C(=O)Nc2cc(C(=O)O)ccn2)[C@@H](c2ccc(C)cc2Cl)N=C(Nc2nc3ccc(F)cc3o2)N1] | 1.93 | 6.85 | >30 | <1 | 53.8 |
| | (S)-2-(4-(2-chloro-4-methylphenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 57 | 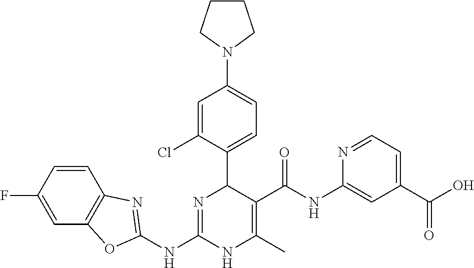 [SMILES: CC1=C(C(=O)Nc2cc(C(=O)O)ccn2)C(c2ccc(N3CCCC3)cc2Cl)N=C(Nc2nc3ccc(F)cc3o2)N1] | 4.09 | 10.86 | >30 | <1 | >87 |
| | 2-(4-(2-chloro-4-(pyrrolidin-1-yl)phenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 58 | 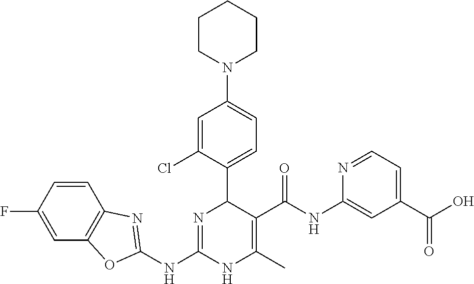 [SMILES: CC1=C(C(=O)Nc2cc(C(=O)O)ccn2)C(c2ccc(N3CCCCC3)cc2Cl)N=C(Nc2nc3ccc(F)cc3o2)N1] | 1.22 | 12.19 | >30 | 1.35 | 1.92 |
| | 2-(4-(2-chloro-4-(piperidin-1-yl)phenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 59 | 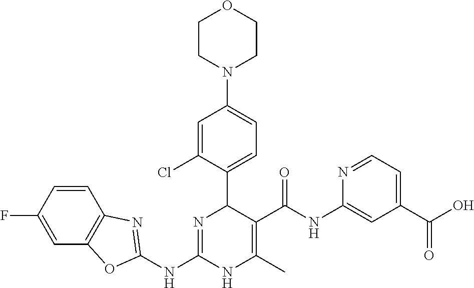 [SMILES: CC1=C(C(=O)Nc2cc(C(=O)O)ccn2)C(c2ccc(N3CCOCC3)cc2Cl)N=C(Nc2nc3ccc(F)cc3o2)N1] | 0.43 | 3.85 | >30 | 26.5 | >89 |
| | 2-(4-(2-chloro-4-morpholinphenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 60 | 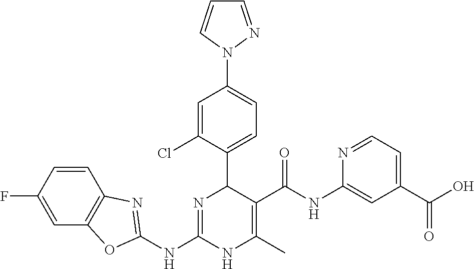 [SMILES: CC1=C(C(=O)Nc2cc(C(=O)O)ccn2)C(c2ccc(-n3cccn3)cc2Cl)N=C(Nc2nc3ccc(F)cc3o2)N1] | 0.77 | 3.43 | >30 | <1 | >87 |
| | 2-(4-(2-chloro-4-(1H-pyrazol-1-yl)phenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 61 | 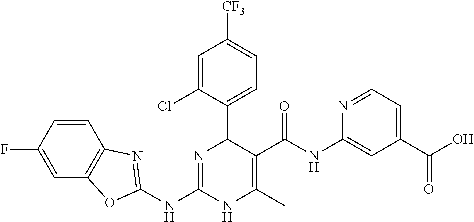 [SMILES: CC1=C(C(=O)Nc2cc(C(=O)O)ccn2)C(c2ccc(C(F)(F)F)cc2Cl)N=C(Nc2nc3ccc(F)cc3o2)N1] | 1.53 | 10.86 | >30 | <1 | 19.4 |
| | 2-(4-(2-chloro-4-(trifluoromethyl)phenyl)-2-((6- | | | | | |
| | 3.85 fluorobenzo[d]oxazol-2-yl)amino)-6- | | | | | |
| | methyl-1,4-dihydropyrimidine-5- | | | | | |
| | carboxamido)isonicotinic acid | | | | | |
| 62 | 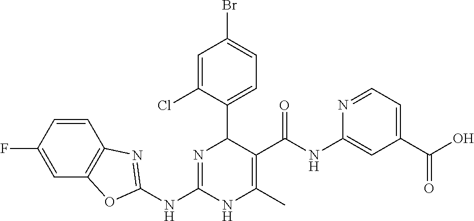 [SMILES: CC1=C(C(=O)Nc2cc(C(=O)O)ccn2)C(c2ccc(Br)cc2Cl)N=C(Nc2nc3ccc(F)cc3o2)N1] | 1.09 | 3.85 | >30 | <1.7 | 2.4 |
| | 2-(4-(4-bromo-2-chlorophenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 63 | 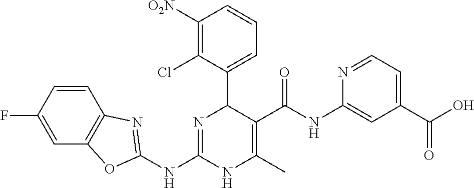 [SMILES: CC1=C(C(=O)Nc2cc(C(=O)O)ccn2)C(c2cccc([N+](=O)[O-])c2Cl)N=C(Nc2nc3ccc(F)cc3o2)N1] | 19.31 (−1.2) | 34.35 (−1.2) | >30 | <1 | >84 |
| | 2-(4-(2-chloro-3-nitrophenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 64 | 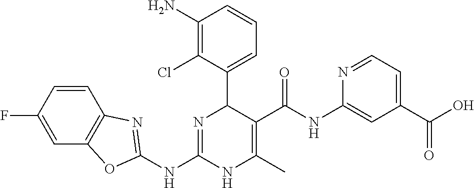 [SMILES: CC1=C(C(=O)Nc2cc(C(=O)O)ccn2)C(c2cccc(N)c2Cl)N=C(Nc2nc3ccc(F)cc3o2)N1] | 3.06 | 17.21 | >30 | <1 | >79 |
| | 2-(4-(3-amino-2-chlorophenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid was | | | | | |
| 65 | 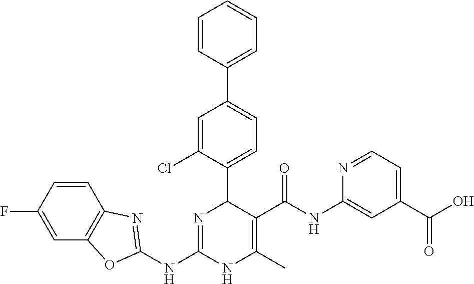 [SMILES: CC1=C(C(=O)Nc2cc(C(=O)O)ccn2)C(c2ccc(-c3ccccc3)cc2Cl)N=C(Nc2nc3ccc(F)cc3o2)N1] | 3.434 | | >30 | <4.9 | <1 |
| | 2-(4-(3-chloro-[1,1′-biphenyl]-4-yl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 66 | 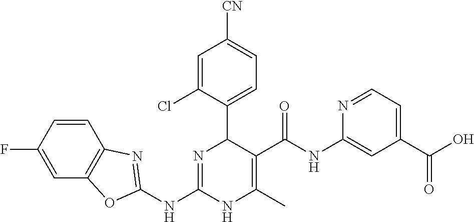 [SMILES: CC1=C(C(=O)Nc2cc(C(=O)O)ccn2)C(c2ccc(C#N)cc2Cl)N=C(Nc2nc3ccc(F)cc3o2)N1] | 0.61 | 3.85 | >30 | <1 | >81 |
| | 2-(4-(2-chloro-4-cyanophenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 67 | 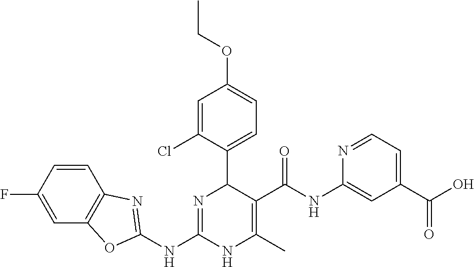 [SMILES: CCOc1ccc(C2N=C(Nc3nc4ccc(F)cc4o3)NC(C)=C2C(=O)Nc2cc(C(=O)O)ccn2)c(Cl)c1] | 0.69 | 3.85 | >30 | <1 | 11.3 |
| | 2-(4-(2-chloro-4-ethoxyphenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 68 | 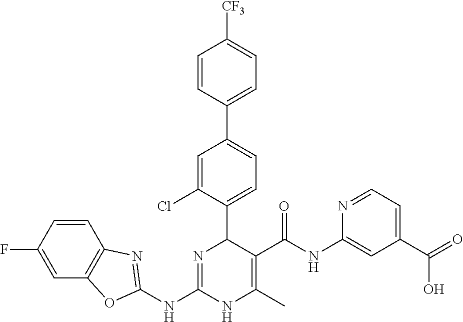 [SMILES: CC1=C(C(=O)Nc2cc(C(=O)O)ccn2)C(c2ccc(-c3ccc(C(F)(F)F)cc3)cc2Cl)N=C(Nc2nc3ccc(F)cc3o2)N1] | 7.69 | 21.67 | >30 | 6.6 | 8.1 |
| | 2-(4-(3-chloro-4′-(trifluoromethyl)-[1,1′- | | | | | |
| | biphenyl]-4-yl)-2-((6-fluorobenzo[d]oxazol-2- | | | | | |
| | yl)amino)-6-methyl-1,4-dihydropyrimidine-5- | | | | | |
| | carboxamido)isonicotinic acid | | | | | |
| 69 | 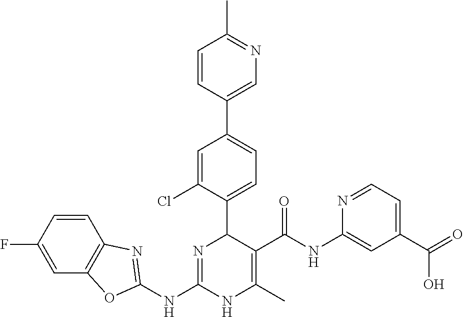 [SMILES: CC1=C(C(=O)Nc2cc(C(=O)O)ccn2)C(c2ccc(-c3ccc(C)nc3)cc2Cl)N=C(Nc2nc3ccc(F)cc3o2)N1] | 0.34 | 1.22 | >30 | <1 | >90 |
| | 2-(4-(2-chloro-4-(6-methylpyridin-3-yl)phenyl)- | | | | | |
| | 2-((6-fluorobenzo[d]oxazol-2-yl)amino)-6- | | | | | |
| | methyl-1,4-dihydropyrimidine-5- | | | | | |
| | carboxamido)isonicotinic acid | | | | | |
| 70 | 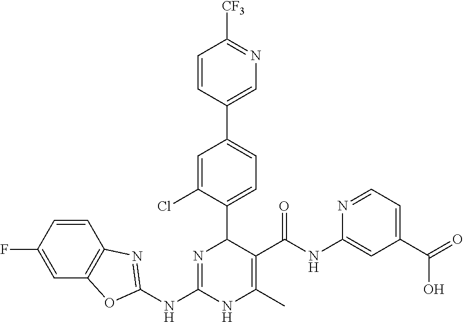 [SMILES: CC1=C(C(=O)Nc2cc(C(=O)O)ccn2)C(c2ccc(-c3ccc(C(F)(F)F)nc3)cc2Cl)N=C(Nc2nc3ccc(F)cc3o2)N1] | 1.21 | 3.85 | >30 | <1 | 28.6 |
| | 2-(4-(2-chloro-4-(6-(trifluoromethyl)pyridin-3- | | | | | |
| | yl)phenyl)-2-((6-fluorobenzo[d]oxazol-2- | | | | | |
| | yl)amino)-6-methyl-1,4-dihydropyrimidine-5- | | | | | |
| | carboxamido)isonicotinic acid | | | | | |
| 71 | 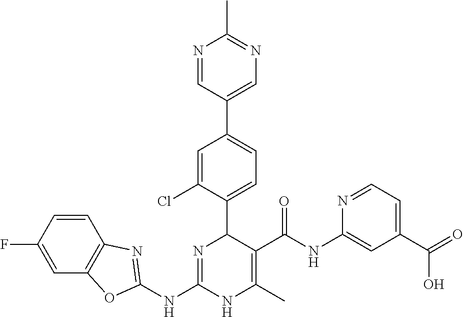 [SMILES: CC1=C(C(=O)Nc2cc(C(=O)O)ccn2)C(c2ccc(-c3cnc(C)nc3)cc2Cl)N=C(Nc2nc3ccc(F)cc3o2)N1] | 0.11 | 0.69 | >30 | <1 | >90 |
| | 2-(4-(2-chloro-4-(2-methylpyrimidin-5- | | | | | |
| | yl)phenyl)-2-((6-fluorobenzo[d]oxazol-2- | | | | | |
| | yl)amino)-6-methyl-1,4-dihydropyrimidine-5- | | | | | |
| | carboxamido)isonicotinic acid | | | | | |
| 72 | 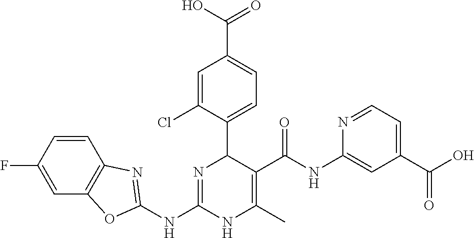 [SMILES: CC1=C(C(=O)Nc2cc(C(=O)O)ccn2)C(c2ccc(C(=O)O)cc2Cl)N=C(Nc2nc3ccc(F)cc3o2)N1] | 2.17 | 15.34 (−1.2) | >30 | <1 | >83 |
| | 2-(4-(4-carboxy-2-chlorophenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 73 | 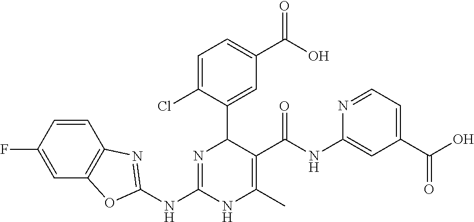 [SMILES: CC1=C(C(=O)Nc2cc(C(=O)O)ccn2)C(c2cc(C(=O)O)ccc2Cl)N=C(Nc2nc3ccc(F)cc3o2)N1] | 1.72 | 12.19 | >30 | 2.85 | >83 |
| | 2-(4-(5-carboxy-2-chlorophenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 74 | 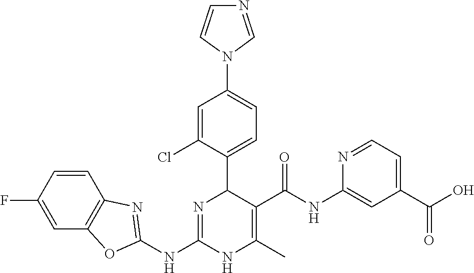 [SMILES: CC1=C(C(=O)Nc2cc(C(=O)O)ccn2)C(c2ccc(-n3ccnc3)cc2Cl)N=C(Nc2nc3ccc(F)cc3o2)N1] | 0.12 | 0.97 | >30 | <1 | >87 |
| | 2-(4-(2-chloro-4-(1H-imidazol-1-yl)phenyl)-2- | | | | | |
| | ((6-fluorobenzo[d]oxazol-2-yl)amino)-6-methyl- | | | | | |
| | 1,4-dihydropyrimidine-5- | | | | | |
| | carboxaido)isonicotinic acid | | | | | |
| 75 | 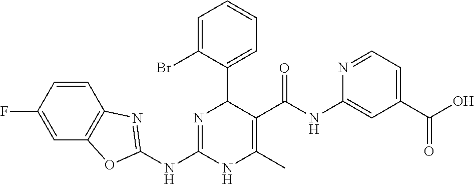 [SMILES: CC1=C(C(=O)Nc2cc(C(=O)O)ccn2)C(c2ccccc2Br)N=C(Nc2nc3ccc(F)cc3o2)N1] | 0.54 | 3.85 | >30 | na | 36.0 |
| | 2-(4-(2-bromophenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 76 | 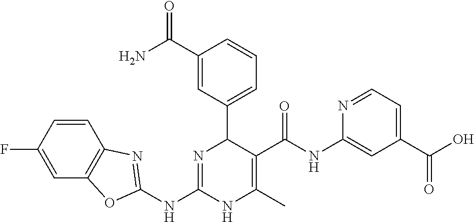 [SMILES: CC1=C(C(=O)Nc2cc(C(=O)O)ccn2)C(c2cccc(C(N)=O)c2)N=C(Nc2nc3ccc(F)cc3o2)N1] | 7.69 | 21.67 (−1.2) | >30 | <1 | >78 |
| | 2-(4-(3-carbamoylphenyl)-2-((6- | | | | | |
| | fluorobenzo[d]oxazol-2-yl)amino)-6-methyl-1,4- | | | | | |
| | dihydropyrimidine-5-carboxamido)isonicotinic | | | | | |
| | acid | | | | | |
| 77 | 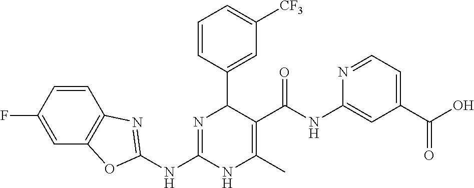 [SMILES: CC1=C(C(=O)Nc2cc(C(=O)O)ccn2)C(c2cccc(C(F)(F)F)c2)N=C(Nc2nc3ccc(F)cc3o2)N1] | 19.31 (−1.2) | in- active | >30 | <1 | 60.1 |
| | 2-(2-((6-fluorobenzo[d]oxazol-2-yl)amino)-6- | | | | | |
| | methyl-4-(3-(trifluoromethyl)phenyl)-1,4- | | | | | |
| | dihydropyrimidine-5-carboxamido)isonicotinic | | | | | |
| | acid | | | | | |
| 78 | 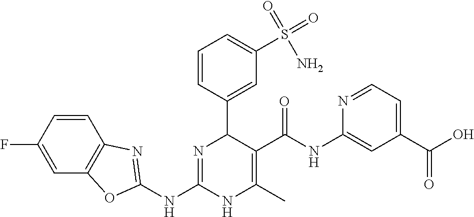 [SMILES: CC1=C(C(=O)Nc2cc(C(=O)O)ccn2)C(c2cccc(S(N)(=O)=O)c2)N=C(Nc2nc3ccc(F)cc3o2)N1] | 17.21 (−1.2) | 38.54 (−2.2) | >30 | na | >84 |
| | 2-(2-((6-fluorobenzo[d]oxazol-2-yl)amino)-6- | | | | | |
| | methyl-4-(3-sulfamoylphenyl)-1,4- | | | | | |
| | dihydropyrimidine-5-carboxamido)isonicotinic | | | | | |
| | acid | | | | | |
| 79 | 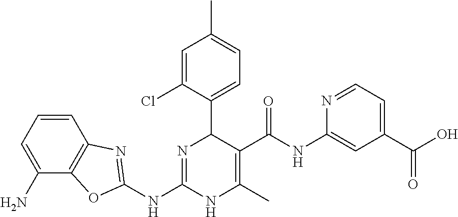 [SMILES: CC1=C(C(=O)Nc2cc(C(=O)O)ccn2)C(c2ccc(C)cc2Cl)N=C(Nc2nc3cccc(N)c3o2)N1] | 0.12 | 1.72 | >30 | <1 | >78 |
| | 2-(2-((7-aminobenzo[d]oxazol-2-yl)amino)-4-(2- | | | | | |
| | chloro-4-methylphenyl)-6-methyl-1,4- | | | | | |
| | dihydropyrimidine-5-carboxamido)isonicotinic | | | | | |
| | acid | | | | | |
| 80 | 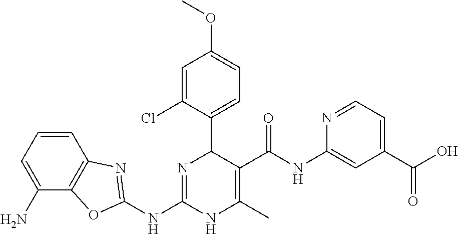 [SMILES: COc1ccc(C2N=C(Nc3nc4cccc(N)c4o3)NC(C)=C2C(=O)Nc2cc(C(=O)O)ccn2)c(Cl)c1] | 0.14 | 1.93 | >30 | <1 | >81 |
| | 2-(2-((7-aminobenzo[d]oxazol-2-yl)amino)-4-(2- | | | | | |
| | chloro-4-methoxyphenyl)-6-methyl-1,4- | | | | | |
| | dihydropyrimidine-5-carboxamido)isonicotinic | | | | | |
| | acid | | | | | |
| 81 | 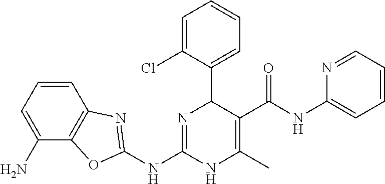 [SMILES: CC1=C(C(=O)Nc2ccccn2)C(c2ccccc2Cl)N=C(Nc2nc3cccc(N)c3o2)N1] | 3.43 | 19.31 (−1.2) | >30 | 271.13 | <1 |
| | 2-((7-aminobenzo[d]oxazol-2-yl)amino)-4-(2- | | | | | |
| | chlorophenyl)-6-methyl-N-(pyridin-2-yl)-1,4- | | | | | |
| | dihydropyrimidine-5-carboxamide | | | | | |
| 82 | 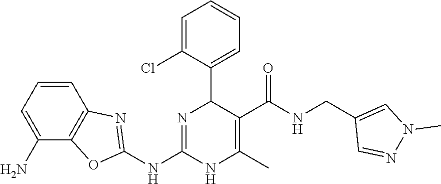 [SMILES: CC1=C(C(=O)NCc2cnn(C)c2)C(c2ccccc2Cl)N=C(Nc2nc3cccc(N)c3o2)N1] | 0.39 | 3.43 | >30 | 101.9 | >72 |
| | 2-((7-aminobenzo[d]oxazol-2-yl)amino)-4-(2- | | | | | |
| | chlorophenyl)-6-methyl-N-((1-methyl-1H- | | | | | |
| | pyrazol-4-yl)methyl)-1,4-dihydropyrimidine-5- | | | | | |
| | carboxamide | | | | | |
| 83 | 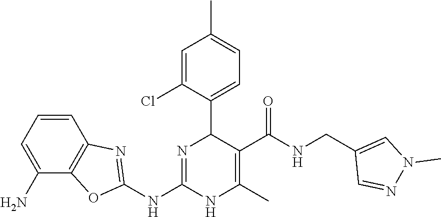 [SMILES: CC1=C(C(=O)NCc2cnn(C)c2)C(c2ccc(C)cc2Cl)N=C(Nc2nc3cccc(N)c3o2)N1] | 0.22 | 2.17 | 14.13 | 25.28 | 4.47 |
| | 2-((7-aminobenzo[d]oxazol-2-yl)amino)-4-(2- | | | | | |
| | chloro-4-methylphenyl)-6-methyl-N-((1-methyl- | | | | | |
| | 1H-pyrazol-4-yl)methyl)-1,4-dihydropyrimidine- | | | | | |
| | 5-carboxamide | | | | | |
| 84 | 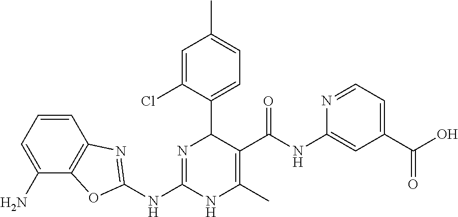 [SMILES: CC1=C(C(=O)Nc2cc(C(=O)O)ccn2)C(c2ccc(C)cc2Cl)N=C(Nc2nc3cccc(N)c3o2)N1] | 3.43 | 21.67 (−1.2) | >30 | <1 | >78 |
| | (S)-2-(2-((7-aminobenzo[d]oxazol-2-yl)amino)- | | | | | |
| | 4-(2-chloro-4-methylphenyl)-6-methyl-1,4- | | | | | |
| | dihydropyrimidine-5-carboxamido)isonicotinic | | | | | |
| | acid | | | | | |
| 85 | 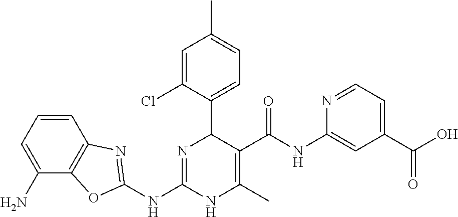 [SMILES: CC1=C(C(=O)Nc2cc(C(=O)O)ccn2)C(c2ccc(C)cc2Cl)N=C(Nc2nc3cccc(N)c3o2)N1] | 0.09 | 1.41 | >30 | <1 | >53 |
| | (R)-2-(2-((7-aminobenzo[d]oxazol-2-yl)amino)- | | | | | |
| | 4-(2-chloro-4-methylphenyl)-6-methyl-1,4- | | | | | |
| | dihydropyrimidine-5-carboxamido)isonicotinic | | | | | |
| | acid | | | | | |
| 86 | 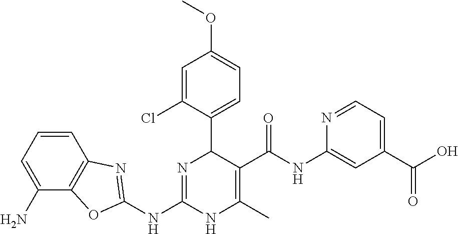 [SMILES: COc1ccc(C2N=C(Nc3nc4cccc(N)c4o3)NC(C)=C2C(=O)Nc2cc(C(=O)O)ccn2)c(Cl)c1] | 5.44 | 34.35 (−2.2) | >30 | <1 | >81 |
| | (S)-2-(2-((7-aminobenzo[d]oxazol-2-yl)amino)- | | | | | |
| | 4-(2-chloro-4-methoxyphenyl)-6-methyl-1,4- | | | | | |
| | dihydropyrimidine-5-carboxamido)isonicotinic | | | | | |
| | acid | | | | | |
| 87 | 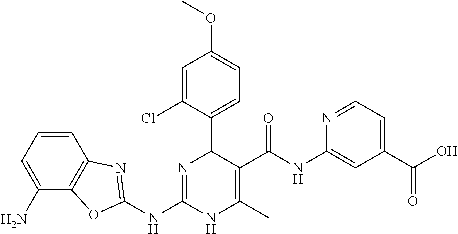 [SMILES: COc1ccc(C2N=C(Nc3nc4cccc(N)c4o3)NC(C)=C2C(=O)Nc2cc(C(=O)O)ccn2)c(Cl)c1] | 0.15 | 2.43 | >30 | <1 | >81 |
| | (R)-2-(2-((7-aminobenzo[d]oxazol-2-yl)amino)- | | | | | |
| | 4-(2-chloro-4-methoxyphenyl)-6-methyl-1,4- | | | | | |
| | dihydropyrimidine-5-carboxamido)isonicotinic | | | | | |
| | acid | | | | | |
| 88 | 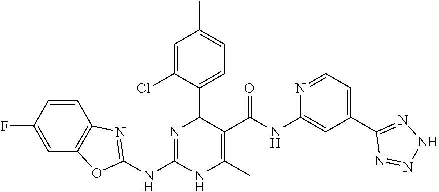 [SMILES: CC1=C(C(=O)Nc2cc(-c3nn[nH]n3)ccn2)C(c2ccc(C)cc2Cl)N=C(Nc2nc3ccc(F)cc3o2)N1] | 1.37 | 9.68 | >30 | <1 | <1 |
| | N-(4-(2H-tetrazol-5-yl)pyridin-2-yl)-4-(2-chloro- | | | | | |
| | 4-methylphenyl)-2-((6-fluorobenzo[d]oxazol-2- | | | | | |
| | yl)amino)-6-methyl-1,4-dihydropyrimidine-5- | | | | | |
| | carboxamide | | | | | |
| 89 | 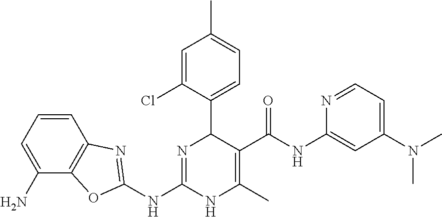 [SMILES: CC1=C(C(=O)Nc2cc(N(C)C)ccn2)C(c2ccc(C)cc2Cl)N=C(Nc2nc3cccc(N)c3o2)N1] | 3.34 | 15.34 (−1.2) | 11.7017 | 179.3 | 4.6 |
| | 2-((7-aminobenzo[d]oxazol-2-yl)amino)-4-(2- | | | | | |
| | chloro-4-methylphenyl)-N-(4- | | | | | |
| | (dimethylamino)pyridin-2-yl)-6-methyl-1,4- | | | | | |
| | dihydropyrimidine-5-carboxamide | | | | | |
| 90 | 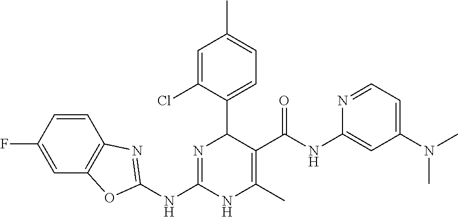 [SMILES: CC1=C(C(=O)Nc2cc(N(C)C)ccn2)C(c2ccc(C)cc2Cl)N=C(Nc2nc3ccc(F)cc3o2)N1] | 4.32 (−1.2) | 6.85 (−1.2) | 17.57836 | 264.3 | 6.2 |
| | 4-(2-chloro-4-methylphenyl)-N-(4- | | | | | |
| | (dimethylamino)pyridin-2-yl)-2-((6- | | | | | |
| | fluorobenzo[d]oxazol-2-yl)amino)-6-methyl-1,4- | | | | | |
| | dihydropyrimidine-5-carboxamide | | | | | |
| 91 | 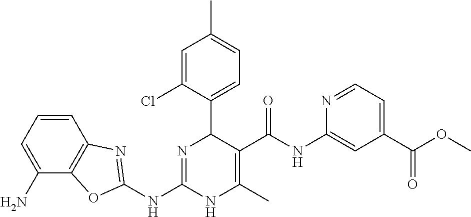 [SMILES: COC(=O)c1ccnc(NC(=O)C2=C(C)NC(Nc3nc4cccc(N)c4o3)=NC2c2ccc(C)cc2Cl)c1] | 2.24 | 7.08 (−1.2) | 25.2784 | 177.1 | <1 |
| | 2-(2-((7-aminobenzo[d]oxazol-2-yl)amino)-4-(2- | | | | | |
| | chloro-4-methylphenyl)-6-methyl-1,4- | | | | | |
| | dihydropyrimidine-5-carboxamido)isonicotinate | | | | | |
| 92 | 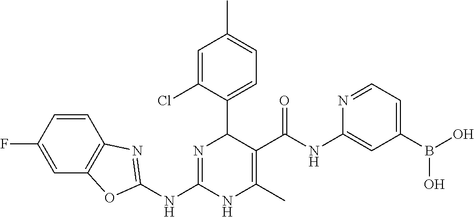 [SMILES: CC1=C(C(=O)Nc2cc(B(O)O)ccn2)C(c2ccc(C)cc2Cl)N=C(Nc2nc3ccc(F)cc3o2)N1] | 1.93 | 10.86 | 14.12714 | <3.2 | <1 |
| | (2-(4-(2-chloro-4-methylphenyl)-2-((6- | | | | | |
| | fluorobenzo[d]oxazol-2-yl)amino)-6-methyl-1,4- | | | | | |
| | dihydropyrimidine-5-carboxamido)pyridin-4- | | | | | |
| | yl)boronic acid | | | | | |
| 93 | 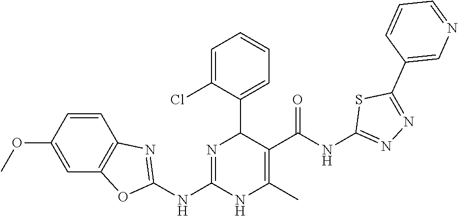 [SMILES: COc1ccc2nc(NC3=NC(c4ccccc4Cl)C(C(=O)Nc4nnc(-c5cccnc5)s4)=C(C)N3)oc2c1] | 9.68 (−1.4) | 21.67 (4) | >30 | <4.4 | <1 |
| | 4-(2-chlorophenyl)-2-((6- | | | | | |
| | methoxybenzo[d]oxazol-2-yl)amino)-6-methyl- | | | | | |
| | N-(5-(pyridin-3-yl)-1,3,4-thiadiazol-2-yl)-1,4- | | | | | |
| | dihydropyrimidine-5-carboxamide | | | | | |
| 94 | 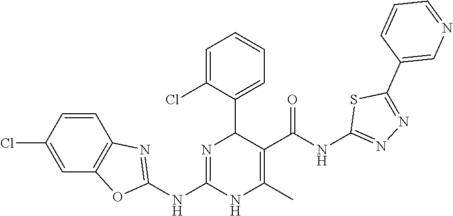 [SMILES: CC1=C(C(=O)Nc2nnc(-c3cccnc3)s2)C(c2ccccc2Cl)N=C(Nc2nc3ccc(Cl)cc3o2)N1] | 27.28 (−1.2) | in- active | >30 | <10.3 | <1 |
| | 2-((6-chlorobenzo[d]oxazol-2-yl)amino)-4-(2- | | | | | |
| | chlorophenyl)-6-methyl-N-(5-(pyridin-3-yl)- | | | | | |
| | 1,3,4-thiadiazol-2-yl)-1,4-dihydropyrimidine-5- | | | | | |
| | carboxamide | | | | | |
| 95 | 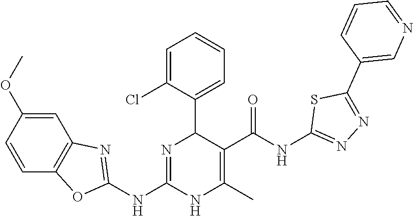 [SMILES: COc1ccc2oc(NC3=NC(c4ccccc4Cl)C(C(=O)Nc4nnc(-c5cccnc5)s4)=C(C)N3)nc2c1] | in- active | in- active | >30 | <19.1 | <1 |
| | 4-(2-chlorophenyl)-2-((5- | | | | | |
| | methoxybenzo[d]oxazol-2-yl)amino)-6-methyl- | | | | | |
| | N-(5-(pyridin-3-yl)-1,3,4-thiadiazol-2-yl)-1,4- | | | | | |
| | dihydropyrimidine-5-carboxamide | | | | | |
| 96 | 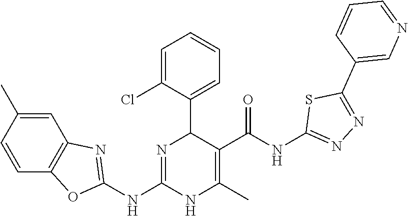 [SMILES: CC1=C(C(=O)Nc2nnc(-c3cccnc3)s2)C(c2ccccc2Cl)N=C(Nc2nc3cc(C)ccc3o2)N1] | 4.85 (−1.2) | 10.86 (−1.2) | >30 | <1.5 | <1 |
| | 4-(2-chlorophenyl)-2-((5-methylbenzo[d]oxazol- | | | | | |
| | 2-yl)amino)-6-methyl-N-(5-(pyridin-3-yl)-1,3,4- | | | | | |
| | thiadiazol-2-yl)-1,4-dihydropyrimidine-5- | | | | | |
| | carboxamide | | | | | |
| 97 | 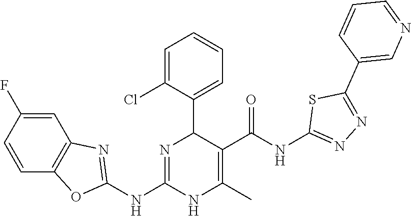 [SMILES: CC1=C(C(=O)Nc2nnc(-c3cccnc3)s2)C(c2ccccc2Cl)N=C(Nc2nc3cc(F)ccc3o2)N1] | 5.44 (−1.2) | 19.31 (−1.2) | >30 | <1.5 | <1 |
| | 4-(2-chlorophenyl)-2-((5-fluorobenzo[d]oxazol- | | | | | |
| | 2-yl)amino)-6-methyl-N-(5-(pyridin-3-yl)-1,3,4- | | | | | |
| | thiadiazol-2-yl)-1,4-dihydropyrimidine-5- | | | | | |
| | carboxamide | | | | | |
| 98 | 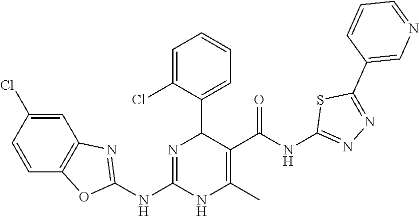 [SMILES: CC1=C(C(=O)Nc2nnc(-c3cccnc3)s2)C(c2ccccc2Cl)N=C(Nc2nc3cc(Cl)ccc3o2)N1] | 6.11 (−1.2) | 19.31 (−1.2) | >30 | <3.4 | <1 |
| | 4-(2-chlorophenyl)-2-((5-chlorobenzo[d]oxazol- | | | | | |
| | 2-yl)amino)-6-methyl-N-(5-pyridin-3-yl)-1,3,4- | | | | | |
| | thiadiazol-2-yl)-1,4-dihydropyrimidine-5- | | | | | |
| | carboxamide | | | | | |
| 99 | 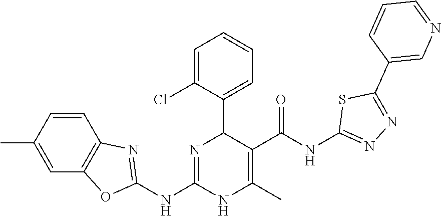 [SMILES: CC1=C(C(=O)Nc2nnc(-c3cccnc3)s2)C(c2ccccc2Cl)N=C(Nc2nc3ccc(C)cc3o2)N1] | in- active | in- active | >30 | <2.9 | <1 |
| | 4-(2-chlorophenyl)-2-((6-methylbenzo[d]oxazol- | | | | | |
| | 2-yl)amino)-6-methyl-N-(5-(pyridin-3-yl)-1,3,4- | | | | | |
| | thiadiazol-2-yl)-1,4-dihydropyrimidine-5- | | | | | |
| | carboxamide | | | | | |
| 100 | 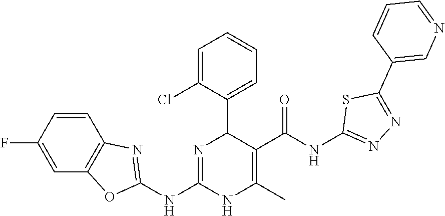 [SMILES: CC1=C(C(=O)Nc2nnc(-c3cccnc3)s2)C(c2ccccc2Cl)N=C(Nc2nc3ccc(F)cc3o2)N1] | 17.21 | 38.54 (−1.2) | >30 | <9.9 | <1 |
| | 4-(2-chlorophenyl)-2-((6-fluorobenzo[d]oxazol- | | | | | |
| | 2-yl)amino)-6-methyl-N-(5-(pyridin-3-yl)-1,3,4- | | | | | |
| | thiadiazol-2-yl)-1,4-dihydropyrimidine-5- | | | | | |
| | carboxamide | | | | | |
| 101 | 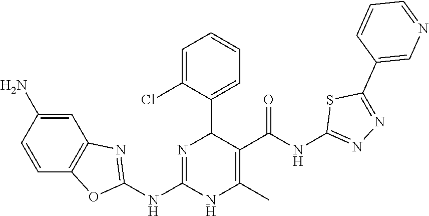 [SMILES: CC1=C(C(=O)Nc2nnc(-c3cccnc3)s2)C(c2ccccc2Cl)N=C(Nc2nc3cc(N)ccc3o2)N1] | 10.86 (−1.2) | 43.24 (−1.2) | >30 | 4.4 | 1.8 |
| | 2-((5-aminobenzo[d]oxazol-2-yl)amino)-4-(2- | | | | | |
| | chlorophenyl)-6-methyl-N-(5-(pyridin-3-yl)- | | | | | |
| | 1,3,4-thiadiazol-2-yl)-1,4-dihydropyrimidine-5- | | | | | |
| | carboxamide | | | | | |
| 102 | 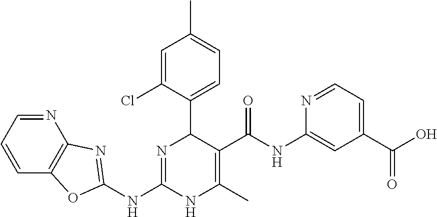 [SMILES: CC1=C(C(=O)Nc2cc(C(=O)O)ccn2)C(c2ccc(C)cc2Cl)N=C(Nc2nc3ncccc3o2)N1] | 4.85 | 19.31 (−1.2) | >30 | <1 | >51 |
| | 2-(4-(2-chloro-4-methylphenyl)-6-methyl-2- | | | | | |
| | (oxazolo[4,5-b]pyridin-2-ylamino)-1,4- | | | | | |
| | dihydropyrimidine-5-carboxamido)isonicotinic | | | | | |
| | acid | | | | | |
| 103 | 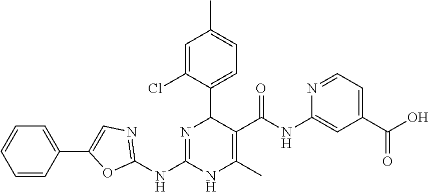 [SMILES: CC1=C(C(=O)Nc2cc(C(=O)O)ccn2)C(c2ccc(C)cc2Cl)N=C(Nc2ncc(-c3ccccc3)o2)N1] | 24.32 (−1.2) | 43.24 (−1.2) | >30 | 4.14 | 42.2 |
| | 2-(4-(2-chloro-4-methylphenyl)-6-methyl-2-((5- | | | | | |
| | phenyloxazol-2-yl)amino)-1,4- | | | | | |
| | dihydropyrimidine-5-carboxamido)isonicotinic | | | | | |
| | acid | | | | | |
| 104 | 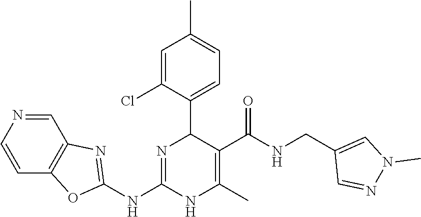 [SMILES: CC1=C(C(=O)NCc2cnn(C)c2)C(c2ccc(C)cc2Cl)N=C(Nc2nc3cnccc3o2)N1] | 3.06 | 12.19 (−1.2) | 5.12 | 127.62 | >49 |
| | 4-(2-chloro-4-methylphenyl)-6-methyl-N-((1- | | | | | |
| | methyl-1H-pyrazol-4-yl)methyl)-2-(oxazolo[4,5- | | | | | |
| | c]pyridin-2-ylamino)-1,4-dihydropyrimidine-5- | | | | | |
| | carboxamide | | | | | |
| 105 | 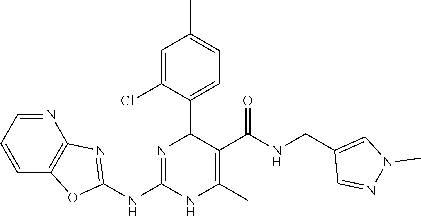 [SMILES: CC1=C(C(=O)NCc2cnn(C)c2)C(c2ccc(C)cc2Cl)N=C(Nc2nc3ncccc3o2)N1] | 15.34 (−1.2) | in- active | 6.14863 | 62.84 | >49 |
| | 4-(2-chloro-4-methylphenyl)-6-methyl-N-((1- | | | | | |
| | methyl-1H-pyrazol-4-yl)methyl)-2-(oxazolo[4,5- | | | | | |
| | b]pyridin-2-ylamino)-1,4-dihydropyrimidine-5- | | | | | |
| | carboxamide | | | | | |
| 106 | 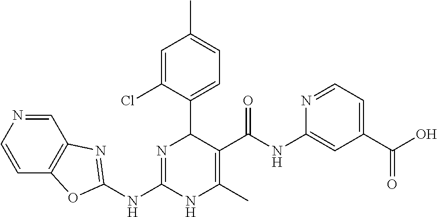 [SMILES: CC1=C(C(=O)Nc2cc(C(=O)O)ccn2)C(c2ccc(C)cc2Cl)N=C(Nc2nc3cnccc3o2)N1] | 0.86 | 6.85 | >30 | <1 | >51 |
| | 2-(4-(2-chloro-4-methylphenyl)-6-methyl-2- | | | | | |
| | (oxazolo[4,5-c]pyridin-2-ylamino)-1,4- | | | | | |
| | dihydropyrimidine-5-carboxamido)isonicotinic | | | | | |
| | acid | | | | | |
| 107 | 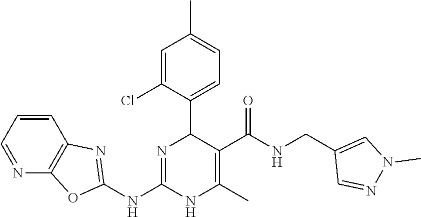 [SMILES: CC1=C(C(=O)NCc2cnn(C)c2)C(c2ccc(C)cc2Cl)N=C(Nc2nc3cccnc3o2)N1] | 17.21 (−1.2) | in- active | 3.66421 | 355.44 | 4.8 |
| | 4-(2-chloro-4-methylphenyl)-6-methyl-N-((1- | | | | | |
| | methyl-1H-pyrazol-4-yl)methyl)-2-(oxazolo[5,4- | | | | | |
| | b]pyridin-2-ylamino)-1,4-dihydropyrimidine-5- | | | | | |
| | carboxamide | | | | | |
| 108 | 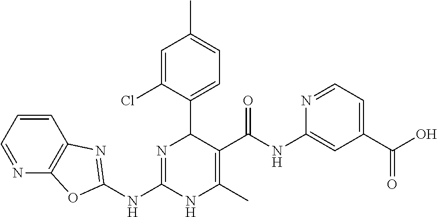 [SMILES: CC1=C(C(=O)Nc2cc(C(=O)O)ccn2)C(c2ccc(C)cc2Cl)N=C(Nc2nc3cccnc3o2)N1] | 12.19 (−1.2) | in- active | >30 | <1 | >51 |
| | 2-(4-(2-chloro-4-methylphenyl)-6-methyl-2- | | | | | |
| | (oxazolo[5,4-b]pyridin-2-ylamino)-1,4- | | | | | |
| | dihydropyrimidine-5-carboxamido)isonicotinic | | | | | |
| | acid | | | | | |
| 109 | 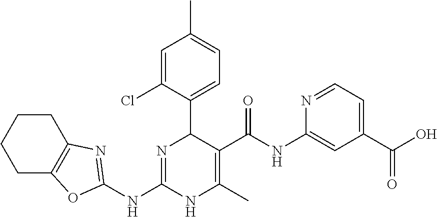 [SMILES: CC1=C(C(=O)Nc2cc(C(=O)O)ccn2)C(c2ccc(C)cc2Cl)N=C(Nc2nc3c(o2)CCCC3)N1] | 3.06 | 4.32 (−1.2) | >30 | <1.8 | >52 |
| | 2-(4-(2-chloro-4-methylphenyl)-6-methyl-2- | | | | | |
| | ((4,5,6,7-tetrahydrobenzo[d]oxazol-2-yl)amino)- | | | | | |
| | 1,4-dihydropyrimidine-5- | | | | | |
| | carboxamido)isonicotinic acid | | | | | |
| 110 | 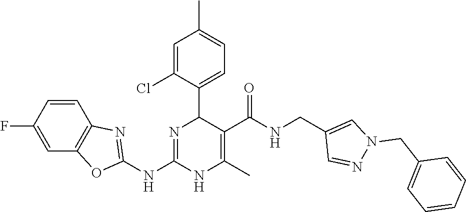 [SMILES: CC1=C(C(=O)NCc2cnn(Cc3ccccc3)c2)C(c2ccc(C)cc2Cl)N=C(Nc2nc3ccc(F)cc3o2)N1] | 1.72 | 5.44 (−1.2) | 2.74 | 159.0 | <1 |
| | N-((1-benzyl-1H-pyrazol-4-yl)methyl)-4-(2- | | | | | |
| | chloro-4-methylphenyl)-2-((6- | | | | | |
| | fluorobenzo[d]oxazol-2-yl)amino)-6-methyl-1,4- | | | | | |
| | dihydropyrimidine-5-carboxamide | | | | | |
| 111 | 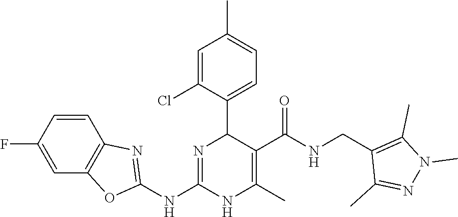 [SMILES: CC1=C(C(=O)NCc2c(C)nn(C)c2C)C(c2ccc(C)cc2Cl)N=C(Nc2nc3ccc(F)cc3o2)N1] | 3.85 | 4.85 | 2.66 | 16.91 | <1 |
| | 4-(2-chloro-4-methylphenyl)-2-((6- | | | | | |
| | fluorobenzo[d]oxazol-2-yl)amino)-6-methyl-N- | | | | | |
| | ((1,3,5-trimethyl-1H-pyrazol-4-yl)methyl)-1,4- | | | | | |
| | dihydropyrimidine-5-carboxamide | | | | | |
| 112 | 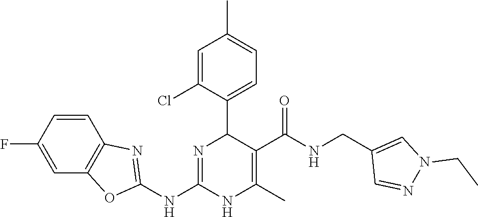 [SMILES: CCn1cc(CNC(=O)C2=C(C)NC(Nc3nc4ccc(F)cc4o3)=NC2c2ccc(C)cc2Cl)cn1] | 1.22 | 6.85 | 9.22 | <21.9 | <1 |
| | 4-(2-chloro-4-methylphenyl)-N-((1-ethyl-1H- | | | | | |
| | pyrazol-4-yl)methyl)-2-((6- | | | | | |
| | fluorobenzo[d]oxazol-2-yl)amino)-6-methyl-1,4- | | | | | |
| | dihydropyrimidine-5-carboxamide | | | | | |
| 113 | 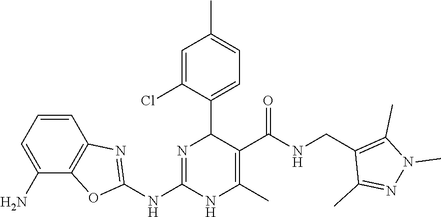 [SMILES: CC1=C(C(=O)NCc2c(C)nn(C)c2C)C(c2ccc(C)cc2Cl)N=C(Nc2nc3cccc(N)c3o2)N1] | 1.22 | 6.85 | 2.36915 | 127.92 | 12.0 |
| | 2-((7-aminobenzo[d]oxazol-2-yl)amino)-4-(2- | | | | | |
| | chloro-4-methylphenyl)-6-methyl-N-((1,3,5- | | | | | |
| | trimethyl-1H-pyrazol-4-yl)methyl)-1,4- | | | | | |
| | dihydropyrimidine-5-carboxamide | | | | | |
| 114 | 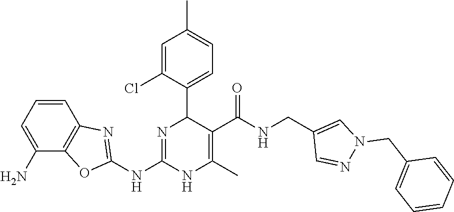 [SMILES: CC1=C(C(=O)NCc2cnn(Cc3ccccc3)c2)C(c2ccc(C)cc2Cl)N=C(Nc2nc3cccc(N)c3o2)N1] | 0.39 | 5.44 (−1.2) | 6.731962 | 103.8 | <1 |
| | 2-((7-aminobenzo[d]oxazol-2-yl)amino)-N-((1- | | | | | |
| | benzyl-1H-pyrazol-4-yl)methyl)-4-(2-chloro-4- | | | | | |
| | methylphenyl)-6-methyl-1,4-dihydropyrimidine- | | | | | |
| | 5-carboxamide | | | | | |
| 115 | 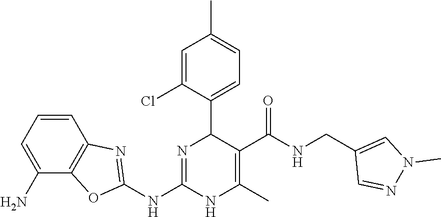 [SMILES: CC1=C(C(=O)NCc2cnn(C)c2)C(c2ccc(C)cc2Cl)N=C(Nc2nc3cccc(N)c3o2)N1] | 0.13 | 1.58 | 18.91319 | 416.8 | 17.14 |
| | (R)-2-((7-aminobenzo[d]oxazol-2-yl)amino)-4- | | | | | |
| | (2-chloro-4-methylphenyl)-6-methyl-N-((1- | | | | | |
| | methyl-1H-pyrazol-4-yl)methyl)-1,4- | | | | | |
| | dihydropyrimidine-5-carboxamide | | | | | |
| 116 | 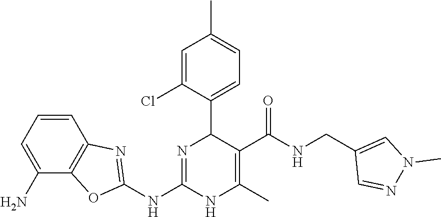 [SMILES: CC1=C(C(=O)NCc2cnn(C)c2)C(c2ccc(C)cc2Cl)N=C(Nc2nc3cccc(N)c3o2)N1] | 12.19 | 30.61 (−1.2) | 7.86771 | 326.9 | 21.9 |
| | (S)-2-((7-aminobenzo[d]oxazol-2-yl)amino)-4- | | | | | |
| | (2-chloro-4-methylphenyl)-6-methyl-N-((1- | | | | | |
| | methyl-1H-pyrazol-4-yl)methyl)-1,4- | | | | | |
| | dihydropyrimidine-5-carboxamide | | | | | |
| 117 | 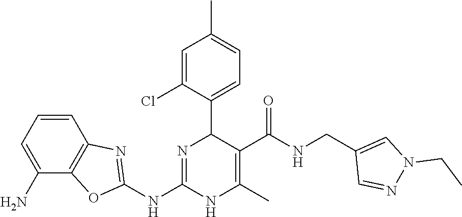 [SMILES: CCn1cc(CNC(=O)C2=C(C)NC(Nc3nc4cccc(N)c4o3)=NC2c2ccc(C)cc2Cl)cn1] | 0.17 | 3.06 | 11.30284 | 360.4 | 2.43 |
| | 2-((7-aminobenzo[d]oxazol-2-yl)amino)-4-(2- | | | | | |
| | chloro-4-methylphenyl)-6-methyl-N-((1-ethyl- | | | | | |
| | 1H-pyrazol-4-yl)methyl)-1,4-dihydropyrimidine- | | | | | |
| | 5-carboxamide | | | | | |
| 118 | 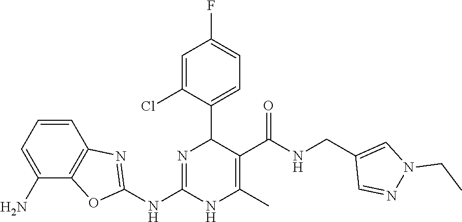 [SMILES: CCn1cc(CNC(=O)C2=C(C)NC(Nc3nc4cccc(N)c4o3)=NC2c2ccc(F)cc2Cl)cn1] | 0.76 | 7.69 | 18.81175 | 188.9 | na |
| | 2-((7-aminobenzo[d]oxazol-2-yl)amino)-4-(2- | | | | | |
| | chloro-4-fluorophenyl)-N-((1-ethyl-1H-pyrazol- | | | | | |
| | 4-yl)methyl)-6-methyl-1,4-dihydropyrimidine-5- | | | | | |
| | carboxamide | | | | | |
| 119 | 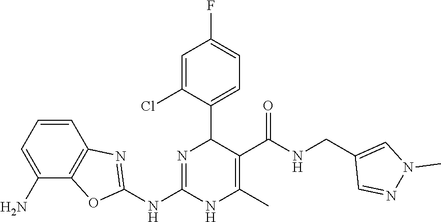 [SMILES: CC1=C(C(=O)NCc2cnn(C)c2)C(c2ccc(F)cc2Cl)N=C(Nc2nc3cccc(N)c3o2)N1] | 0.68 | 6.85 | 29.59115 | 17.14 | 49.6 |
| | 2-((7-aminobenzo[d]oxazol-2-yl)amino)-4-(2- | | | | | |
| | chloro-4-fluorophenyl)-N-((1-methyl-1H- | | | | | |
| | pyrazol-4-yl)methyl)-6-methyl-1,4- | | | | | |
| | dihydropyrimidine-5-carboxamide | | | | | |
| 120 | 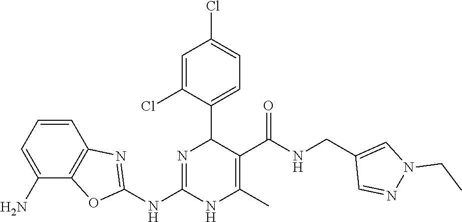 [SMILES: CCn1cc(CNC(=O)C2=C(C)NC(Nc3nc4cccc(N)c4o3)=NC2c2ccc(Cl)cc2Cl)cn1] | 0.48 | 8.63 | 14.13858 | 289.5 | 4.6 |
| | 2-((7-aminobenzo[d]oxazol-2-yl)amino)-4-(2,4- | | | | | |
| | dichlorophenyl)-N-((1-ethyl-1H-pyrazol-4- | | | | | |
| | yl)methyl)-6-methyl-1,4-dihydropyrimidine-5- | | | | | |
| | carboxamide | | | | | |
| 121 | 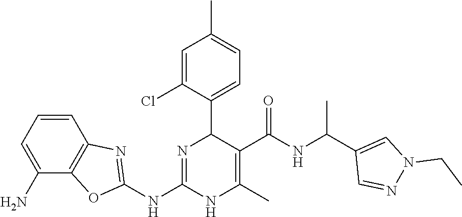 [SMILES: CCn1cc(C(C)NC(=O)C2=C(C)NC(Nc3nc4cccc(N)c4o3)=NC2c2ccc(C)cc2Cl)cn1] | 0.05 | 1.09 | 17.9283 | 332.7 | <1 |
| | 2-((7-aminobenzo[d]oxazol-2-yl)amino)-4-(2- | | | | | |
| | chloro-4-methylphenyl)-6-methyl-N-((1-ethyl- | | | | | |
| | 1H-pyrazol-4-yl)methyl)-1,4-dihydropyrimidine- | | | | | |
| | 5-carboxamide | | | | | |
| 122 | 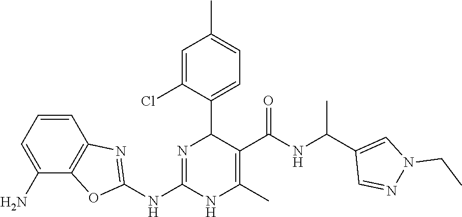 [SMILES: CCn1cc(C(C)NC(=O)C2=C(C)NC(Nc3nc4cccc(N)c4o3)=NC2c2ccc(C)cc2Cl)cn1] | 0.54 | 5.44 | 15.10075 | 114.1 | <1 |
| | 2-((7-aminobenzo[d]oxazol-2-yl)amino)-4-(2- | | | | | |
| | chloro-4-methylphenyl)-6-methyl-N-((1-ethyl- | | | | | |
| | 1H-pyrazol-4-yl)methyl)-1,4-dihydropyrimidine- | | | | | |
| | 5-carboxamide | | | | | |
| 123 | 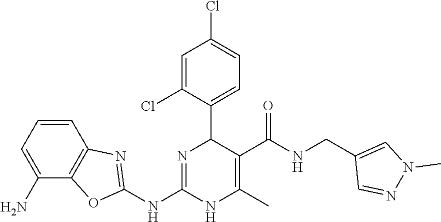 [SMILES: CC1=C(C(=O)NCc2cnn(C)c2)C(c2ccc(Cl)cc2Cl)N=C(Nc2nc3cccc(N)c3o2)N1] | 1.09 | 6.85 | 17.76226 | 142.4 | 15.63 |
| | 2-((7-aminobenzo[d]oxazol-2-yl)amino)-4-(2,4- | | | | | |
| | dichlorophenyl)-N-((1-methyl-1H-pyrazol-4- | | | | | |
| | yl)methyl)-6-methyl-1,4-dihydropyrimidine-5- | | | | | |
| | carboxamide | | | | | |
| 124 | 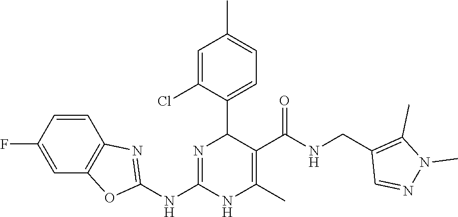 [SMILES: CC1=C(C(=O)NCc2cnn(C)c2C)C(c2ccc(C)cc2Cl)N=C(Nc2nc3ccc(F)cc3o2)N1] | 0.49 | 3.43 | 2.99286 | 460.2 | <1 |
| | 4-(2-chloro-4-methylphenyl)-N-((1,4-dimethyl- | | | | | |
| | 1H-pyrazol-4-yl)methyl)-2-((6- | | | | | |
| | fluorobenzo[d]oxazol-2-yl)amino)-6-methyl-1,4- | | | | | |
| | dihydropyrimidine-5-carboxamide | | | | | |
| 125 | 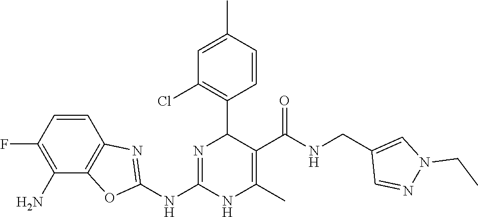 [SMILES: CCn1cc(CNC(=O)C2=C(C)NC(Nc3nc4ccc(F)c(N)c4o3)=NC2c2ccc(C)cc2Cl)cn1] | 0.05 | 0.61 | 14.07509 | 410.3 | <1 |
| | 2-((7-amino-6-fluorobenzo[d]oxazol-2- | | | | | |
| | yl)amino)-4-(2-chloro-4-methylphenyl)-N-((1- | | | | | |
| | ethyl-1H-pyrazol-4-yl)methyl)-6-methyl-1,4- | | | | | |
| | dihydropyrimidine-5-carboxamide | | | | | |
| 126 | 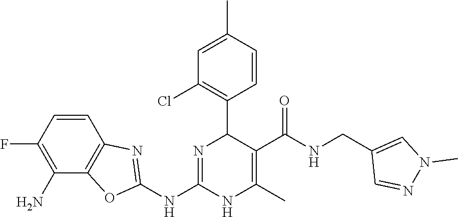 [SMILES: CC1=C(C(=O)NCc2cnn(C)c2)C(c2ccc(C)cc2Cl)N=C(Nc2nc3ccc(F)c(N)c3o2)N1] | 0.05 | 0.69 | 17.10456 | 459.1 | 1.27 |
| | 2-((7-amino-6-fluorobenzo[d]oxazol-2- | | | | | |
| | yl)amino)-4-(2-chloro-4-methylphenyl)-6- | | | | | |
| | methyl-N-((1-methyl-1H-pyrazol-4-yl)methyl)- | | | | | |
| | 1,4-dihydropyrimidine-5-carboxamide | | | | | |
| 127 | 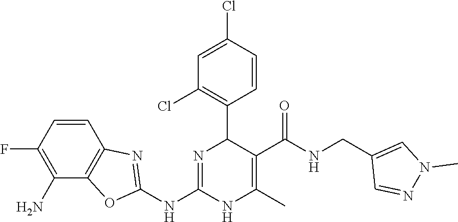 [SMILES: CC1=C(C(=O)NCc2cnn(C)c2)C(c2ccc(Cl)cc2Cl)N=C(Nc2nc3ccc(F)c(N)c3o2)N1] | 0.22 | 1.93 | >30 | 166.7 | 5.9 |
| | 2-((7-amino-6-fluorobenzo[d]oxazol-2- | | | | | |
| | yl)amino)-4-(2,4-dichlorophenyl)-6-methyl-N- | | | | | |
| | ((1-methyl-1H-pyrazol-4-yl)methyl)-1,4- | | | | | |
| | dihydropyrimidine-5-carboxamide | | | | | |
| 128 | 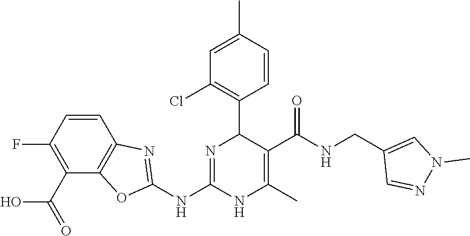 [SMILES: CC1=C(C(=O)NCc2cnn(C)c2)C(c2ccc(C)cc2Cl)N=C(Nc2nc3ccc(F)c(C(=O)O)c3o2)N1] | 17.21 (−1.2) | 21.67 (−1.2) | >30 | <1 | 26.3 |
| | 2-((4-(2-chloro-4-methylphenyl)-6-methyl-5- | | | | | |
| | (((1-methyl-1H-pyrazol-4-yl)methyl)carbamoyl)- | | | | | |
| | 1,4-dihydropyrimidin-2-yl)amino)-6- | | | | | |
| | fluorobenzo[d]oxazole-7-carboxylic acid | | | | | |
| 129 | 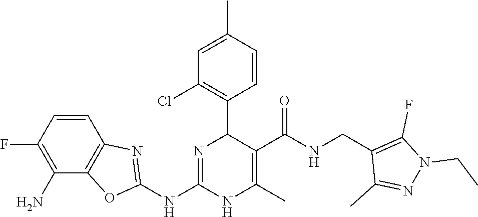 [SMILES: CCn1nc(C)c(CNC(=O)C2=C(C)NC(Nc3nc4ccc(F)c(N)c4o3)=NC2c2ccc(C)cc2Cl)c1F] | 0.43 | 2.17 | 3.54974 | 187.9 | 1.53 |
| | 2-((7-amino-6-fluorobenzo[d]oxazol-2- | | | | | |
| | yl)amino)-4-(2-chloro-4-methylphenyl)-N-((1- | | | | | |
| | ethyl-5-fluoro-3-methyl-1H-pyrazol-4- | | | | | |
| | yl)methyl)-6-methyl-1,4-dihydropyrimidine-5-carboxamide | | | | | |
| 130 | 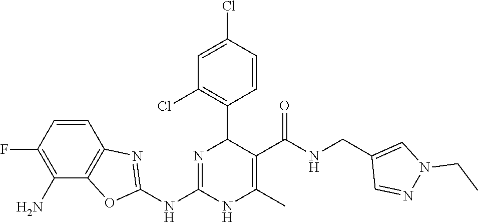 [SMILES: CCn1cc(CNC(=O)C2=C(C)NC(Nc3nc4ccc(F)c(N)c4o3)=NC2c2ccc(Cl)cc2Cl)cn1] | 0.27 | 1.93 | 6.78721 | 380.3 | <1 |
| | 2-((7-amino-6-fluorobenzo[d]oxazol-2- | | | | | |
| | yl)amino)-4-(2,4-dichlorophenyl)-N-((1-ethyl- | | | | | |
| | 1H-pyrazol-4-yl)methyl)-6-methyl-1,4- | | | | | |
| | dihydropyrimidine-5-carboxamide | | | | | |
| 131 | 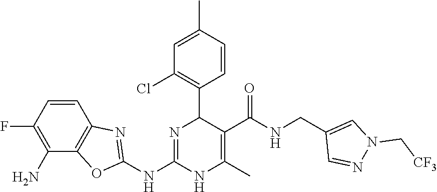 [SMILES: CC1=C(C(=O)NCc2cnn(CC(F)(F)F)c2)C(c2ccc(C)cc2Cl)N=C(Nc2nc3ccc(F)c(N)c3o2)N1] | 0.19 | 3.06 | 18.68961 | 193.1 | 1.56 |
| | 2-((7-amino-6-fluorobenzo[d]oxazol-2- | | | | | |
| | yl)amino)-4-(2-chloro-4-methylphenyl)-6- | | | | | |
| | methyl-N-((1-(2,2,2-trifluoroethyl)-1H-pyrazol-4- | | | | | |
| | yl)methyl)-1,4-dihydropyrimidine-5-carboxamide | | | | | |
| 132 | 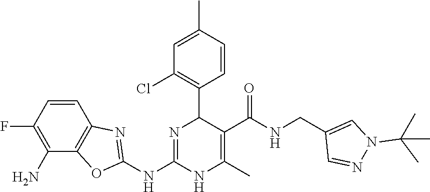 [SMILES: CC1=C(C(=O)NCc2cnn(C(C)(C)C)c2)C(c2ccc(C)cc2Cl)N=C(Nc2nc3ccc(F)c(N)c3o2)N1] | 0.77 | 4.32 | 14.40801 | 553.2 | <1 |
| | 2-((7-amino-6-fluorobenzo[d]oxazol-2- | | | | | |
| | yl)amino)-N-((1-(tert-butyl)-1H-pyrazol-4- | | | | | |
| | yl)methyl)-4-(2-chloro-4-methylphenyl)-6- | | | | | |
| | methyl-1,4-dihydropyrimidine-5-carboxamide | | | | | |
| 133 | 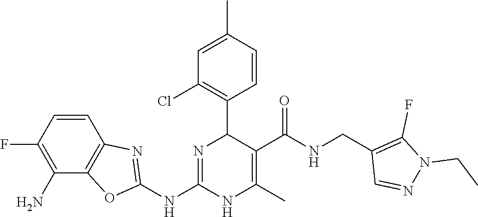 [SMILES: CCn1ncc(CNC(=O)C2=C(C)NC(Nc3nc4ccc(F)c(N)c4o3)=NC2c2ccc(C)cc2Cl)c1F] | 0.11 | 1.09 | 7.03646 | 427.4 | <1 |
| | 2-((7-amino-6-fluorobenzo[d]oxazol-2- | | | | | |
| | yl)amino)-4-(2-chloro-4-methylphenyl)-N-((1- | | | | | |
| | ethyl-5-fluoro-1H-pyrazol-4-yl)methyl)-6- | | | | | |
| | methyl-1,4-dihydropyrimidine-5-carboxamide | | | | | |
| 134 | 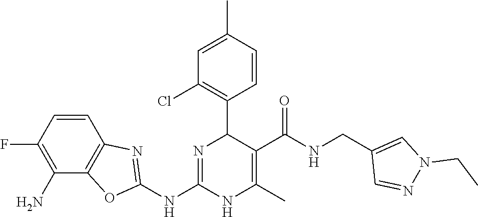 [SMILES: CCn1cc(CNC(=O)C2=C(C)NC(Nc3nc4ccc(F)c(N)c4o3)=NC2c2ccc(C)cc2Cl)cn1] | 1.53 | 10.86 (−1.2) | 8.61894 | 772.8 | <1 |
| | (S)-2-((7-amino-6-fluorobenzo[d]oxazol-2- | | | | | |
| | yl)amino)-4-(2-chloro-4-methylphenyl)-N-((1- | | | | | |
| | ethyl-1H-pyrazol-4-yl)methyl)-6-methyl-1,4- | | | | | |
| | dihydropyrimidine-5-carboxamide | | | | | |
| 135 | 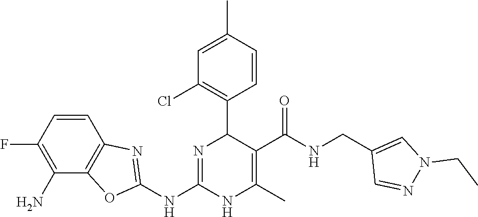 [SMILES: CCn1cc(CNC(=O)C2=C(C)NC(Nc3nc4ccc(F)c(N)c4o3)=NC2c2ccc(C)cc2Cl)cn1] | 0.07 | 0.97 | na | 407.39 | 2.05 |
| | (R)-2-((7-amino-6-fluorobenzo[d]oxazol-2- | | | | | |
| | yl)amino)-4-(2-chloro-4-methylphenyl)-N-((1- | | | | | |
| | ethyl-1H-pyrazol-4-yl)methyl)-6-methyl-1,4- | | | | | |
| | dihydropyrimidine-5-carboxamide | | | | | |
| 136 | 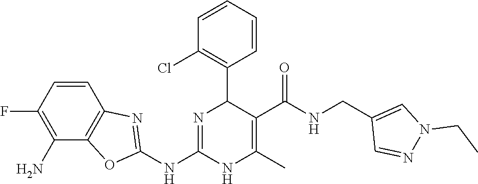 [SMILES: CCn1cc(CNC(=O)C2=C(C)NC(Nc3nc4ccc(F)c(N)c4o3)=NC2c2ccccc2Cl)cn1] | 0.19 | 2.17 | 17.72457 | 133.5 | 23.87079 |
| | 2-((7-amino-6-fluorobenzo[d]oxazol-2- | | | | | |
| | yl)amino)-4-(2-chlorophenyl)-N-((1-ethyl-1H- | | | | | |
| | pyrazol-4-yl)methyl)-6-methyl-1,4- | | | | | |
| | dihydropyrimidine-5-carboxamide | | | | | |
| 137 | 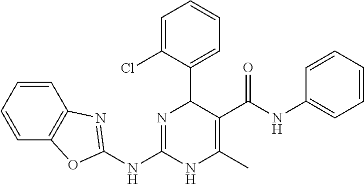 [SMILES: CC1=C(C(=O)Nc2ccccc2)C(c2ccccc2Cl)N=C(Nc2nc3ccccc3o2)N1] | 10.86 (−1.2) | 13.67 (−1.2) | 24 | na | <1 |
| | 2-(benzo[d]oxazol-2-ylamino)-4-(2- | | | | | |
| | chlorophenyl)-6-methyl-N-phenyl-1,4- | | | | | |
| | dihydropyrimidine-5-carboxamide | | | | | |
| 138 | 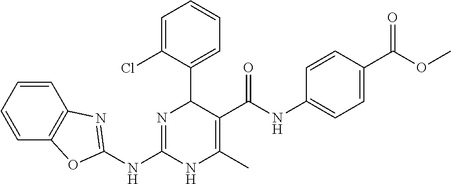 [SMILES: COC(=O)c1ccc(NC(=O)C2=C(C)NC(Nc3nc4ccccc4o3)=NC2c2ccccc2Cl)cc1] | 4.32 (−1.2) | 9.68 (−1.2) | 17.7182 | 25.4 | <1 |
| | 4-(2-(benzo[d]oxazol-2-ylamino)-4-(2- | | | | | |
| | chlorophenyl)-6-methyl-1,4-dihydropyrimidine- | | | | | |
| | 5-carboxamido)benzoate | | | | | |
| 139 | 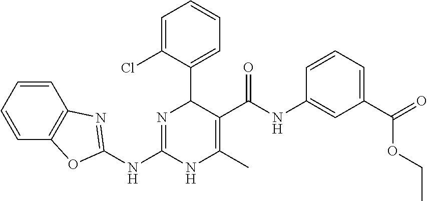 [SMILES: CCOC(=O)c1cccc(NC(=O)C2=C(C)NC(Nc3nc4ccccc4o3)=NC2c2ccccc2Cl)c1] | 3.85 (−1.2) | na | >30 | 8.8 | <1 |
| | ethyl 3-(2-(benzo[d]oxazol-2-ylamino)-4-(2- | | | | | |
| | chlorophenyl)-6-methyl-1,4-dihydropyrimidine- | | | | | |
| | 5-carboxamido)benzoate | | | | | |
| 140 | 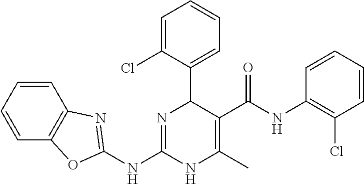 [SMILES: CC1=C(C(=O)Nc2ccccc2Cl)C(c2ccccc2Cl)N=C(Nc2nc3ccccc3o2)N1] | 4.32 (−1.2) | in- active | 28.73652 | 56.0 | <1 |
| | 2-(benzo[d]oxazol-2-ylamino)-N,4-bis(2- | | | | | |
| | chlorophenyl)-6-methyl-1,4-dihydropyrimidine- | | | | | |
| | 5-carboxamide | | | | | |
| 141 | 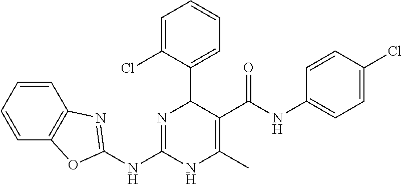 [SMILES: CC1=C(C(=O)Nc2ccc(Cl)cc2)C(c2ccccc2Cl)N=C(Nc2nc3ccccc3o2)N1] | in- active | 7.69 (−1.2) | >30 | 2.0 | <1 |
| | 2-(benzo[d]oxazol-2-ylamino)-4-(2- | | | | | |
| | chlorophenyl)-N-(4-chlorophenyl)-6-methyl-1,4- | | | | | |
| | dihydropyrimidine-5-carboxamide | | | | | |
| 142 | 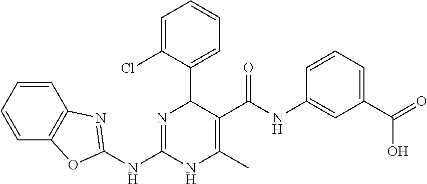 [SMILES: CC1=C(C(=O)Nc2cccc(C(=O)O)c2)C(c2ccccc2Cl)N=C(Nc2nc3ccccc3o2)N1] | 2.43 | 6.11 | >30 | <1 | 59.1 |
| | 3-(2-(benzo[d]oxazol-2-ylamino)-4-(2- | | | | | |
| | chlorophenyl)-6-methyl-1,4-dihydropyrimidine- | | | | | |
| | 5-carboxamido)benzoic acid | | | | | |
| 143 | 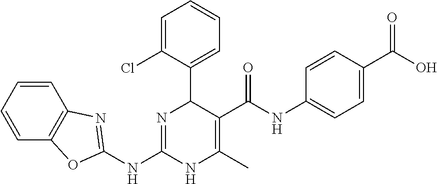 [SMILES: CC1=C(C(=O)Nc2ccc(C(=O)O)cc2)C(c2ccccc2Cl)N=C(Nc2nc3ccccc3o2)N1] | 1.53 | 6.11 | >30 | 3.6 | <1 |
| | 4-(2-(benzo[d]oxazol-2-ylamino)-4-(2- | | | | | |
| | chlorophenyl)-6-methyl-1,4-dihydropyrimidine- | | | | | |
| | 5-carboxamido)benzoic acid | | | | | |
| 144 | 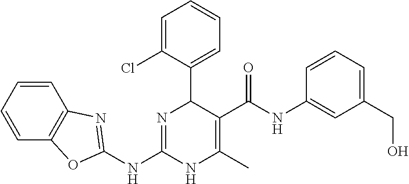 [SMILES: CC1=C(C(=O)Nc2cccc(CO)c2)C(c2ccccc2Cl)N=C(Nc2nc3ccccc3o2)N1] | 3.85 | 9.68 | >30 | 1124.1 | <1 |
| | 2-(benzo[d]oxazol-2-ylamino)-4-(2- | | | | | |
| | chlorophenyl)-N-(3-(hydroxymethyl)phenyl)-6- | | | | | |
| | methyl-1,4-dihydropyrimidine-5-carboxamide | | | | | |
| 145 | 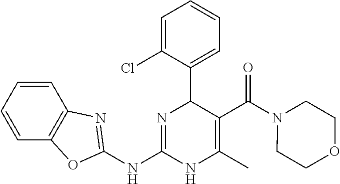 [SMILES: CC1=C(C(=O)N2CCOCC2)C(c2ccccc2Cl)N=C(Nc2nc3ccccc3o2)N1] | 24.32 (−1.2) | 24.32 (−1.2) | 2.1 | 1046.7 | 15.3 |
| | (2-(benzo[d]oxazol-2-ylamino)-4-(2- | | | | | |
| | chlorophenyl)-6-methyl-1,4-dihydropyrimidin-5- | | | | | |
| | yl)(morpholino)methanone | | | | | |
| 146 | 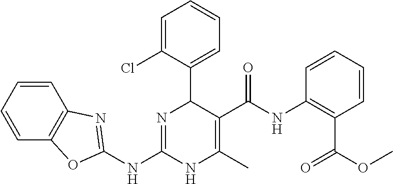 [SMILES: COC(=O)c1ccccc1NC(=O)C1=C(C)NC(Nc2nc3ccccc3o2)=NC1c1ccccc1Cl] | in- active | in- active | na | na | na |
| | 2-(2-(benzo[d]oxazol-2-ylamino)-4-(2- | | | | | |
| | chlorophenyl)-6-methyl-1,4-dihydropyrimidine- | | | | | |
| | 5-carboxamido)benzoate | | | | | |
| 147 | 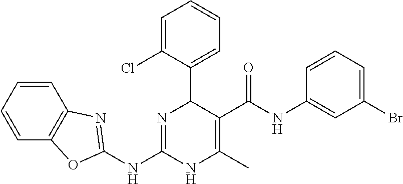 [SMILES: CC1=C(C(=O)Nc2cccc(Br)c2)C(c2ccccc2Cl)N=C(Nc2nc3ccccc3o2)N1] | in- active | in- active | >30 | <1.8 | <1 |
| | 2-(benzo[d]oxazol-2-ylamino)-N-(3- | | | | | |
| | bromophenyl)-4-(2-chlorophenyl)-6-methyl-1,4- | | | | | |
| | dihydropyrimidine-5-carboxamide | | | | | |
| 148 | 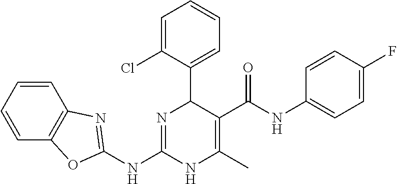 [SMILES: CC1=C(C(=O)Nc2ccc(F)cc2)C(c2ccccc2Cl)N=C(Nc2nc3ccccc3o2)N1] | 7.69 (−1.2) | 12.19 (−1.2) | >30 | 29.4 | <1 |
| | 2-(benzo[d]oxazol-2-ylamino)-N-(4- | | | | | |
| | fluorophenyl)-4-(2-chlorophenyl)-6-methyl-1,4- | | | | | |
| | dihydropyrimidine-5-carboxamide | | | | | |
| 149 | 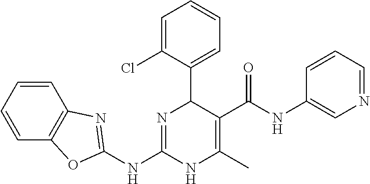 [SMILES: CC1=C(C(=O)Nc2cccnc2)C(c2ccccc2Cl)N=C(Nc2nc3ccccc3o2)N1] | 6.85 | 12.19 | 20 | 513.0 | <1 |
| | 2-(benzo[d]oxazol-2-ylamino)-N-(pyridin-3-yl)- | | | | | |
| | 4-(2-chlorophenyl)-6-methyl-1,4- | | | | | |
| | dihydropyrimidine-5-carboxamide | | | | | |
| 150 | 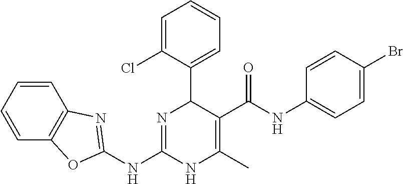 [SMILES: CC1=C(C(=O)Nc2ccc(Br)cc2)C(c2ccccc2Cl)N=C(Nc2nc3ccccc3o2)N1] | 9.68 (−1.4) | 15.34 (−1.2) | >30 | <14.9 | <1 |
| | 2-(benzo[d]oxazol-2-ylamino)-N-(4- | | | | | |
| | bromophenyl)-4-(2-chlorophenyl)-6-methyl-1,4- | | | | | |
| | dihydropyrimidine-5-carboxamide | | | | | |
| 151 | 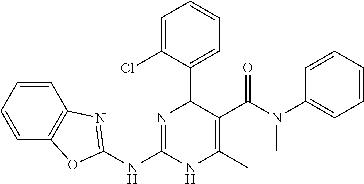 [SMILES: CC1=C(C(=O)N(C)c2ccccc2)C(c2ccccc2Cl)N=C(Nc2nc3ccccc3o2)N1] | in- active | in- active | 1.17567 | 203.1 | <1 |
| | 2-(benzo[d]oxazol-2-ylamino)-4-(2- | | | | | |
| | chlorophenyl)-N,6-dimethyl-N-phenyl-1,4- | | | | | |
| | dihydropyrimidine-5-carboxamide | | | | | |
| 152 | 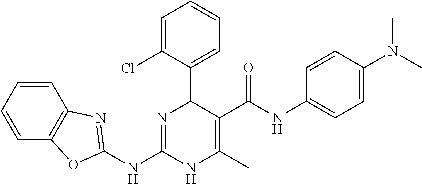 [SMILES: CC1=C(C(=O)Nc2ccc(N(C)C)cc2)C(c2ccccc2Cl)N=C(Nc2nc3ccccc3o2)N1] | 7.69 (−1.2) | 13.67 (−1.2) | 11.45481 | 15.0 | <1 |
| | 2-(benzo[d]oxazol-2-ylamino)-4-(2- | | | | | |
| | chlorophenyl)-N-(4-(dimethylamino)phenyl)-6- | | | | | |
| | methyl-1,4-dihydropyrimidine-5-carboxamide | | | | | |
| 153 | 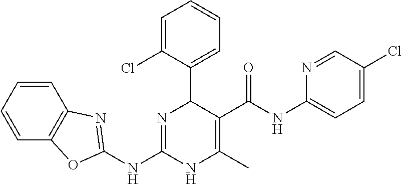 [SMILES: CC1=C(C(=O)Nc2ccc(Cl)cn2)C(c2ccccc2Cl)N=C(Nc2nc3ccccc3o2)N1] | 4.85 (−1.2) | in- active | 30.78616 | na | <1 |
| | 2-(benzo[d]oxazol-2-ylamino)-4-(2- | | | | | |
| | chlorophenyl)-N-(5-chloropyridin-2-yl)-6- | | | | | |
| | methyl-1,4-dihydropyrimidine-5-carboxamide | | | | | |
| 154 | 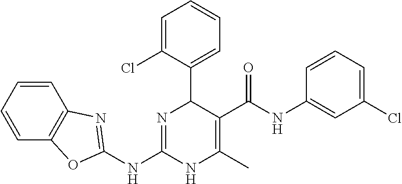 [SMILES: CC1=C(C(=O)Nc2cccc(Cl)c2)C(c2ccccc2Cl)N=C(Nc2nc3ccccc3o2)N1] | in- active | in- active | >30 | <9.2 | <1 |
| | 2-(benzo[d]oxazol-2-ylamino)-N-(3- | | | | | |
| | chlorophenyl)-4-(2-chlorophenyl)-6-methyl-1,4- | | | | | |
| | dihydropyrimidine-5-carboxamide | | | | | |
| 155 | 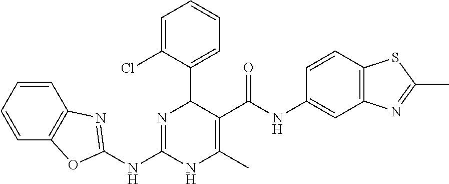 [SMILES: CC1=C(C(=O)Nc2ccc3sc(C)nc3c2)C(c2ccccc2Cl)N=C(Nc2nc3ccccc3o2)N1] | 5.44 (−1.2) | 13.67 (−1.2) | >30 | <4.2 | <1 |
| | 2-(benzo[d]oxazol-2-ylamino)-4-(2- | | | | | |
| | chlorophenyl)-6-methyl-N-(2- | | | | | |
| | methylbenzo[d]thiazol-6-yl)-1,4- | | | | | |
| | dihydropyrimidine-5-carboxamide | | | | | |
| 156 | 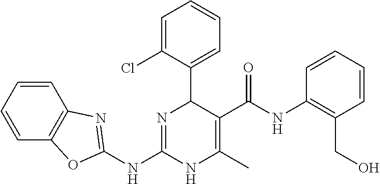 [SMILES: CC1=C(C(=O)Nc2ccccc2CO)C(c2ccccc2Cl)N=C(Nc2nc3ccccc3o2)N1] | 3.85 | 17.21 (−1.2) | 7.70807 | 1038.5 | <1 |
| | 2-(benzo[d]oxazol-2-ylamino)-4-(2- | | | | | |
| | chlorophenyl)-N-(2-(hydroxymethyl)phenyl)-6- | | | | | |
| | methyl-1,4-dihydropyrimidine-5-carboxamide | | | | | |
| 157 | 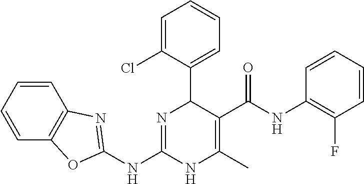 [SMILES: CC1=C(C(=O)Nc2ccccc2F)C(c2ccccc2Cl)N=C(Nc2nc3ccccc3o2)N1] | 7.69 (−1.2) | 9.68 (−1.2) | >30 | na | <1 |
| | 2-(benzo[d]oxazol-2-ylamino)-N-(2- | | | | | |
| | fluorophenyl)-4-(2-chlorophenyl)-6-methyl-1,4- | | | | | |
| | dihydropyrimidine-5-carboxamide | | | | | |
| 158 | 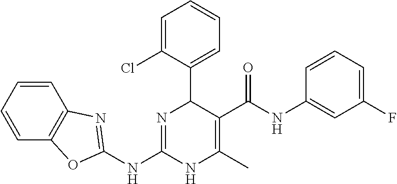 [SMILES: CC1=C(C(=O)Nc2cccc(F)c2)C(c2ccccc2Cl)N=C(Nc2nc3ccccc3o2)N1] | 6.85 (−1.2) | 12.19 (−1.2) | >30 | na | <1 |
| | 2-(benzo[d]oxazol-2-ylamino)-N-(3- | | | | | |
| | fluorophenyl)-4-(2-chlorophenyl)-6-methyl-1,4- | | | | | |
| | dihydropyrimidine-5-carboxamide | | | | | |
| 159 | 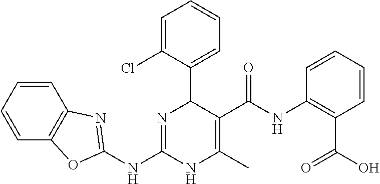 [SMILES: CC1=C(C(=O)Nc2ccccc2C(=O)O)C(c2ccccc2Cl)N=C(Nc2nc3ccccc3o2)N1] | 34.35 (−3) | na | na | na | na |
| | 2-(2-(benzo[d]oxazol-2-ylamino)-4-(2- | | | | | |
| | chlorophenyl)-6-methyl-1,4-dihydropyrimidine- | | | | | |
| | 5-carboxamido)benzoic acid | | | | | |
| 160 | 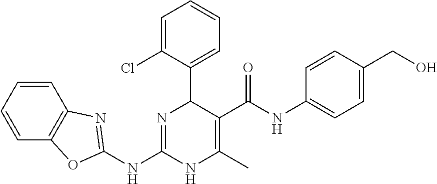 [SMILES: CC1=C(C(=O)Nc2ccc(CO)cc2)C(c2ccccc2Cl)N=C(Nc2nc3ccccc3o2)N1] | 17.21 (−2.2) | na | >30 | 863.9 | <1 |
| | 2-(benzo[d]oxazol-2-ylamino)-4-(2- | | | | | |
| | chlorophenyl)-N-(4-(hydroxymethyl)phenyl)-6- | | | | | |
| | methyl-1,4-dihydropyrimidine-5-carboxamide | | | | | |
| 161 | 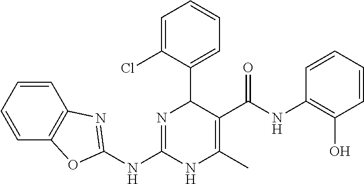 [SMILES: CC1=C(C(=O)Nc2ccccc2O)C(c2ccccc2Cl)N=C(Nc2nc3ccccc3o2)N1] | 6.85 | 17.21 (−1.2) | 17.93522 | 21.1 | <1 |
| | 2-(benzo[d]oxazol-2-ylamino)-N-(2- | | | | | |
| | hydroxyphenyl)-4-(2-chlorophenyl)-6-methyl- | | | | | |
| | 1,4-dihydropyrimidine-5-carboxamide | | | | | |
| 162 | 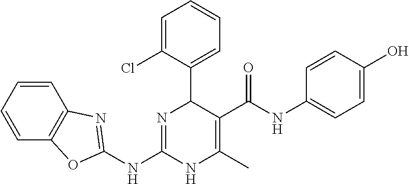 [SMILES: CC1=C(C(=O)Nc2ccc(O)cc2)C(c2ccccc2Cl)N=C(Nc2nc3ccccc3o2)N1] | 3.85 | 9.68 | >30 | <14.5 | na |
| | 2-(benzo[d]oxazol-2-ylamino)-N-(4- | | | | | |
| | hydroxyphenyl)-4-(2-chlorophenyl)-6-methyl- | | | | | |
| | 1,4-dihydropyrimidine-5-carboxamide | | | | | |
| 163 | 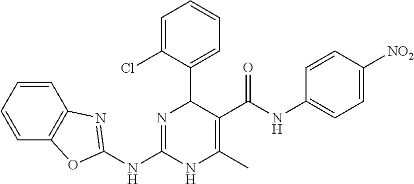 [SMILES: CC1=C(C(=O)Nc2ccc([N+](=O)[O-])cc2)C(c2ccccc2Cl)N=C(Nc2nc3ccccc3o2)N1] | 3.43 (−1.2) | 3.43 (−1.2) | >30 | <7.3 | <1 |
| | 2-(benzo[d]oxazol-2-ylamino)-4-(2- | | | | | |
| | chlorophenyl)-6-methyl-N-(4-nitrophenyl)-1,4- | | | | | |
| | dihydropyrimidine-5-carboxamide | | | | | |
| 164 | 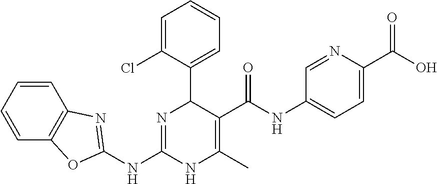 [SMILES: CC1=C(C(=O)Nc2ccc(C(=O)O)nc2)C(c2ccccc2Cl)N=C(Nc2nc3ccccc3o2)N1] | 4.85 | 9.68 | >30 | <1 | 24.6 |
| | 5-(2-(benzo[d]oxazol-2-ylamino)-4-(2- | | | | | |
| | chlorophenyl)-6-methyl-1,4-dihydropyrimidine- | | | | | |
| | 5-carboxamido)picolinic acid | | | | | |
| 165 | 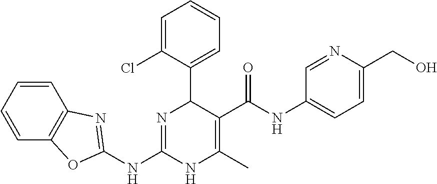 [SMILES: CC1=C(C(=O)Nc2ccc(CO)nc2)C(c2ccccc2Cl)N=C(Nc2nc3ccccc3o2)N1] | 4.85 | 7.69 | >30 | 766.1 | <1 |
| | 2-(benzo[d]oxazol-2-ylamino)-4-(2- | | | | | |
| | chlorophenyl)-N-(6-(hydroxymethyl)pyridin-3- | | | | | |
| | yl)-6-methyl-1,4-dihydropyrimidine-5- | | | | | |
| | carboxamide | | | | | |
| 166 | 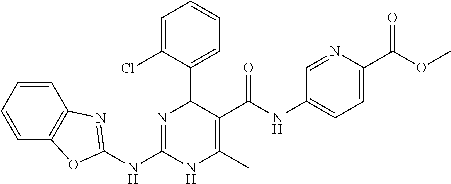 [SMILES: COC(=O)c1ccc(NC(=O)C2=C(C)NC(Nc3nc4ccccc4o3)=NC2c2ccccc2Cl)cn1] | 6.11 | 6.85 | 4.67794 | 96.1 | <1 |
| | methyl 5-(2-(benzo[d]oxazol-2-ylamino)-4-(2- | | | | | |
| | chlorophenyl)-6-methyl-1,4-dihydropyrimidine- | | | | | |
| | 5-carboxamido)picolinate | | | | | |
| 167 | 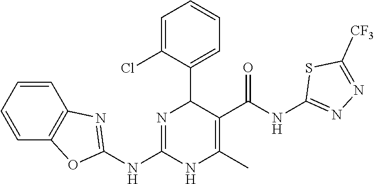 [SMILES: CC1=C(C(=O)Nc2nnc(C(F)(F)F)s2)C(c2ccccc2Cl)N=C(Nc2nc3ccccc3o2)N1] | 12.19 | 21.67 | >30 | 12.6 | <1 |
| | 2-(benzo[d]oxazol-2-ylamino)-4-(2- | | | | | |
| | chlorophenyl)-6-methyl-N-(5-(trifluoromethyl)- | | | | | |
| | 1,3,4-thiadiazol-2-yl)-1,4-dihydropyrimidine-5- | | | | | |
| | carboxamide | | | | | |
| 168 | 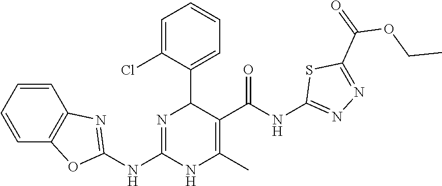 [SMILES: CCOC(=O)c1nnc(NC(=O)C2=C(C)NC(Nc3nc4ccccc4o3)=NC2c2ccccc2Cl)s1] | 4.85 | 10.68 | 3.15753 | <2.7 | <1 |
| | ethyl 5-(2-(benzo[d]oxazol-2-ylamino)-4-(2- | | | | | |
| | chlorophenyl)-6-methyl-1,4-dihydropyrimidine- | | | | | |
| | 5-carboxamido)-1,3,4-thiadiazole-2-carboxylate | | | | | |
| 169 | 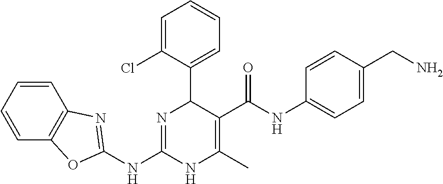 [SMILES: CC1=C(C(=O)Nc2ccc(CN)cc2)C(c2ccccc2Cl)N=C(Nc2nc3ccccc3o2)N1] | 6.11 | 4.32 | 72.47313 | na | 10.0 |
| | N-(4-(aminomethyl)phenyl)-2-(benzo[d]oxazol- | | | | | |
| | 2-ylamino)-4-(2-chlorophenyl)-6-methyl-1,4- | | | | | |
| | dihydropyrimidine-5-carboxamide | | | | | |
| 170 | 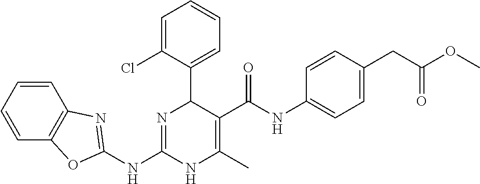 [SMILES: COC(=O)Cc1ccc(NC(=O)C2=C(C)NC(Nc3nc4ccccc4o3)=NC2c2ccccc2Cl)cc1] | 5.44 | 12.19 (−1.2) | 7.46429 | 195.0 | <1 |
| | methyl 2-(4-(2-(benzo[d]oxazol-2-ylamino)-4- | | | | | |
| | (2-chlorophenyl)-6-methyl-1,4- | | | | | |
| | dihydropyrimidine-5- | | | | | |
| | carboxamido)phenyl)acetate | | | | | |
| 171 | 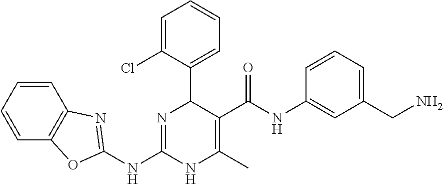 [SMILES: CC1=C(C(=O)Nc2cccc(CN)c2)C(c2ccccc2Cl)N=C(Nc2nc3ccccc3o2)N1] | 10.86 | 6.85 | >30 | 32.7 | 37.5 |
| | N-(3-(aminomethyl)phenyl)-2-(benzo[d]oxazol- | | | | | |
| | 2-ylamino)-4-(2-chlorophenyl)-6-methyl-1,4- | | | | | |
| | dihydropyrimidine-5-carboxamide | | | | | |
| 172 | 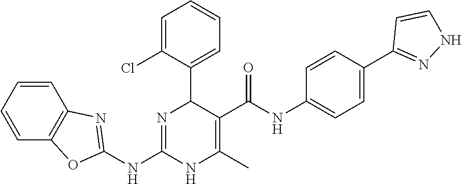 [SMILES: CC1=C(C(=O)Nc2ccc(-c3cc[nH]n3)cc2)C(c2ccccc2Cl)N=C(Nc2nc3ccccc3o2)N1] | 7.69 (−1.2) | in- active | >30 | <24.6 | <1 |
| | N-(3-(1H-pyrazol-3-yl)phenyl)-2- | | | | | |
| | (benzo[d]oxazol-2-ylamino)-4-(2-chlorophenyl)- | | | | | |
| | 6-methyl-1,4-dihydropyrimidine-5-carboxamide | | | | | |
| 173 | 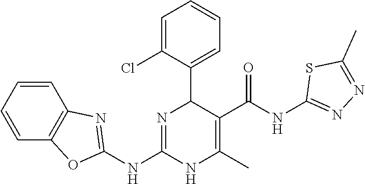 [SMILES: CC1=C(C(=O)Nc2nnc(C)s2)C(c2ccccc2Cl)N=C(Nc2nc3ccccc3o2)N1] | 5.44 | 10.68 (−1.2) | >30 | 23.75 | <1 |
| | 2-(benzo[d]oxazol-2-ylamino)-4-(2- | | | | | |
| | chlorophenyl)-6-methyl-N-(5-methyl-1,3,4- | | | | | |
| | thiadiazol-2-yl)-1,4-dihydropyrimidine-5- | | | | | |
| | carboxamide | | | | | |
| 174 | 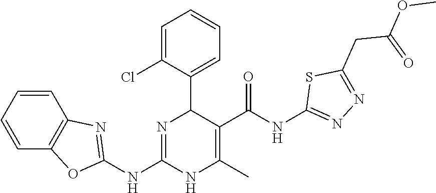 [SMILES: COC(=O)Cc1nnc(NC(=O)C2=C(C)NC(Nc3nc4ccccc4o3)=NC2c2ccccc2Cl)s1] | 6.11 | na | 8.0854 | na | <1 |
| | methyl 2-(5-(2-(benzo[d]oxazol-2-ylamino)-4- | | | | | |
| | (2-chlorophenyl)-6-methyl-1,4- | | | | | |
| | dihydropyrimidine-5-carboxamido)-1,3,4- | | | | | |
| | thiadiazol-2-yl)acetate | | | | | |
| 175 | 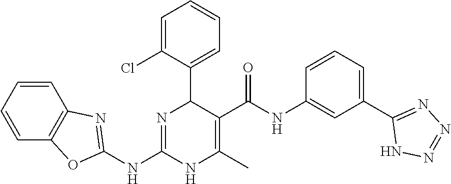 [SMILES: CC1=C(C(=O)Nc2cccc(-c3nnn[nH]3)c2)C(c2ccccc2Cl)N=C(Nc2nc3ccccc3o2)N1] | 19.31 | 34.35 (−2.2) | >30 | 5.0 | 47.5 |
| | N-(3-(1H-tetrahydro-5-yl)phenyl)-2- | | | | | |
| | (benzo[d]oxazol-2-ylamino)-4-(2-chlorophenyl)- | | | | | |
| | 6-methyl-1,4-dihydropyrimidine-5-carboxamide | | | | | |
| 176 | 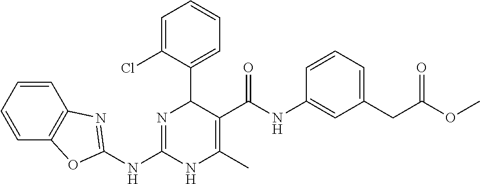 [SMILES: COC(=O)Cc1cccc(NC(=O)C2=C(C)NC(Nc3nc4ccccc4o3)=NC2c2ccccc2Cl)c1] | 10.86 (−1.2) | 13.67 (−1.2) | 10.34404 | <17.4 | <1 |
| | methyl 2-(3-(2-(benzo[d]oxazol-2-ylamino)-4- | | | | | |
| | (2-chlorophenyl)-6-methyl-1,4- | | | | | |
| | dihydropyrimidine-5- | | | | | |
| | carboxamido)phenyl)acetate | | | | | |
| 177 | 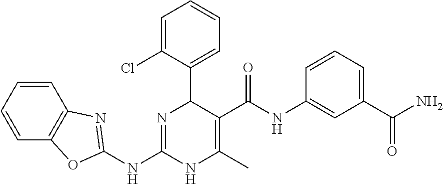 [SMILES: CC1=C(C(=O)Nc2cccc(C(N)=O)c2)C(c2ccccc2Cl)N=C(Nc2nc3ccccc3o2)N1] | 7.69 | 17.21 (−1.2) | >30 | na | <1 |
| | 2-(benzo[d]oxazol-2-ylamino)-N-(3- | | | | | |
| | carbamoylphenyl)-4-(2-chlorophenyl)-6-methyl- | | | | | |
| | 1,4-dihydropyrimidine-5-carboxamide | | | | | |
| 178 | 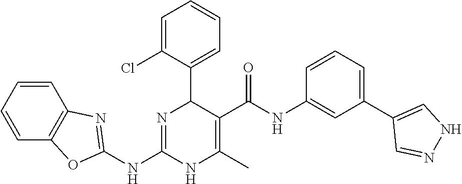 [SMILES: CC1=C(C(=O)Nc2cccc(-c3cn[nH]c3)c2)C(c2ccccc2Cl)N=C(Nc2nc3ccccc3o2)N1] | 17.21 (−1.2) | 38.54 (−2.2) | >30 | <25.4 | <1 |
| | N-(3-(1H-pyrazol-4-yl)phenyl)-2- | | | | | |
| | (benzo[d]oxazol-2-ylamino)-4-(2-chlorophenyl)- | | | | | |
| | 6-methyl-1,4-dihydropyrimidine-5-carboxamide | | | | | |
| 179 | 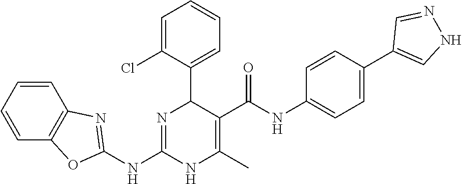 [SMILES: CC1=C(C(=O)Nc2ccc(-c3cn[nH]c3)cc2)C(c2ccccc2Cl)N=C(Nc2nc3ccccc3o2)N1] | 6.85 (−1.2) | 12.19 (−1.2) | na | na | na |
| | N-(4-(1H-pyrazol-4-yl)phenyl)-2- | | | | | |
| | (benzo[d]oxazol-2-ylamino)-4-(2-chlorophenyl)- | | | | | |
| | 6-methyl-1,4-dihydropyrimidine-5-carboxamide | | | | | |
| 180 | 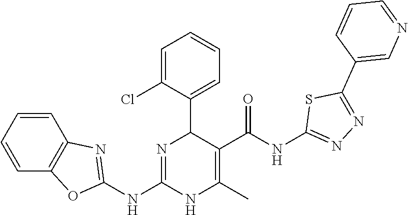 [SMILES: CC1=C(C(=O)Nc2nnc(-c3cccnc3)s2)C(c2ccccc2Cl)N=C(Nc2nc3ccccc3o2)N1] | 3.06 | 9.68 | >30 | <3.7 | <1 |
| | 2-(benzo[d]oxazol-2-ylamino)-4-(2- | | | | | |
| | chlorophenyl)-6-methyl-N-(5-(pyridin-3-yl)- | | | | | |
| | 1,3,4-thiadiazol-2-yl)-1,4-dihydropyrimidine-5- | | | | | |
| | carboxamide | | | | | |
| 181 | 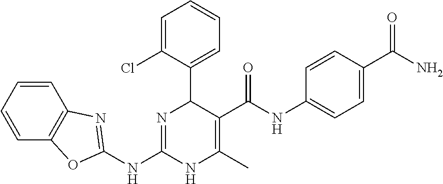 [SMILES: CC1=C(C(=O)Nc2ccc(C(N)=O)cc2)C(c2ccccc2Cl)N=C(Nc2nc3ccccc3o2)N1] | 4.32 | 6.85 | >30 | <6.3 | <1 |
| | 2-(benzo[d]oxazol-2-ylamino)-N-(4- | | | | | |
| | carbamoylphenyl)-4-(2-chlorophenyl)-6-methyl- | | | | | |
| | 1,4-dihydropyrimidine-5-carboxamide | | | | | |
| 182 | 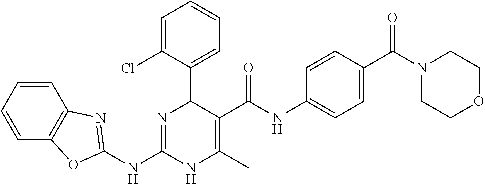 [SMILES: CC1=C(C(=O)Nc2ccc(C(=O)N3CCOCC3)cc2)C(c2ccccc2Cl)N=C(Nc2nc3ccccc3o2)N1] | 19.31 | 21.67 | >30 | na | <1 |
| | 2-(benzo[d]oxazol-2-ylamino)-4-(2- | | | | | |
| | chlorophenyl)-6-methyl-N-(4-(morpholine-4- | | | | | |
| | carbonyl)phenyl)-1,4-dihydropyrimidine-5- | | | | | |
| | carboxamide | | | | | |
| 183 | 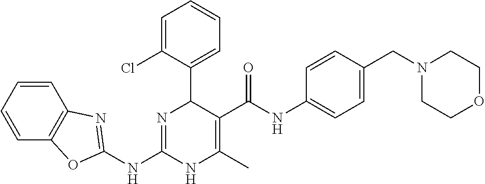 [SMILES: CC1=C(C(=O)Nc2ccc(CN3CCOCC3)cc2)C(c2ccccc2Cl)N=C(Nc2nc3ccccc3o2)N1] | 5.44 | 9.68 (−1.2) | 22.30257 | 120.4 | <1 |
| | 2-(benzo[d]oxazol-2-ylamino)-4-(2- | | | | | |
| | chlorophenyl)-6-methyl-N-(4- | | | | | |
| | (morpholinomethyl)phenyl)-1,4- | | | | | |
| | dihydropyrimidine-5-carboxamide | | | | | |
| 184 | 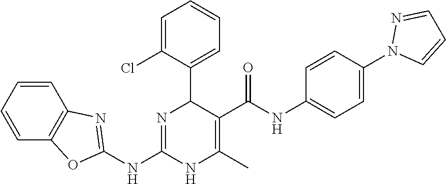 [SMILES: CC1=C(C(=O)Nc2ccc(-n3cccn3)cc2)C(c2ccccc2Cl)N=C(Nc2nc3ccccc3o2)N1] | 6.11 (−1.2) | 12.19 (−1.2) | 16.4271 | <11.8 | <1 |
| | N-(4-(1H-pyrazol-1-yl)phenyl)-2- | | | | | |
| | (benzo[d]oxazol-2-ylamino)-4-(2-chlorophenyl)- | | | | | |
| | 6-methyl-1,4-dihydropyrimidine-5-carboxamide | | | | | |
| 185 | 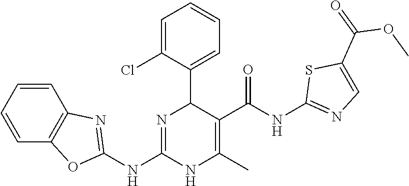 [SMILES: COC(=O)c1cnc(NC(=O)C2=C(C)NC(Nc3nc4ccccc4o3)=NC2c2ccccc2Cl)s1] | 4.32 (−1.2) | 10.86 (−1.2) | 15.91009 | <7.6 | <1 |
| | methyl 2-(2-(benzo[d]oxazol-2-ylamino)-4-(2- | | | | | |
| | chlorophenyl)-6-methyl-1,4-dihydropyrimidine- | | | | | |
| | 5-carboxamido)thiazole-5-carboxylate | | | | | |
| 186 | 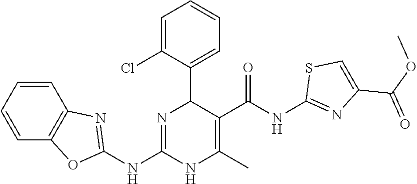 [SMILES: COC(=O)c1csc(NC(=O)C2=C(C)NC(Nc3nc4ccccc4o3)=NC2c2ccccc2Cl)n1] | 12.19 (−1.2) | 13.67 (−1.2) | >30 | 65.6 | <1 |
| | methyl 2-(2-(benzo[d]oxazol-2-ylamino)-4-(2- | | | | | |
| | chlorophenyl)-6-methyl-1,4-dihydropyrimidine- | | | | | |
| | 5-carboxamido)thiazole-4-carboxylate | | | | | |
| 187 | 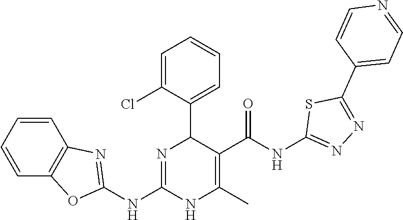 [SMILES: CC1=C(C(=O)Nc2nnc(-c3ccncc3)s2)C(c2ccccc2Cl)N=C(Nc2nc3ccccc3o2)N1] | 10.86 (−1.2) | 19.31 (−1.2) | >30 | 6.2 | <1 |
| | 2-(benzo[d]oxazol-2-ylamino)-4-(2- | | | | | |
| | chlorophenyl)-6-methyl-N-(5-(pyridin-4-yl)- | | | | | |
| | 1,3,4-thiadiazol-2-yl)-1,4-dihydropyrimidine-5- | | | | | |
| | carboxamide | | | | | |
| 188 | 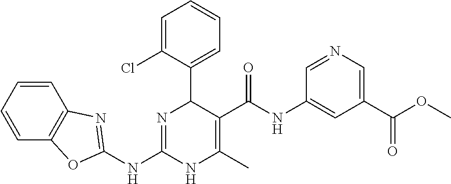 [SMILES: COC(=O)c1cncc(NC(=O)C2=C(C)NC(Nc3nc4ccccc4o3)=NC2c2ccccc2Cl)c1] | 21.67 (−2.1) | na | >30 | 246.9 | <1 |
| | methyl 5-(2-(benzo[d]oxazol-2-ylamino)-4-(2- | | | | | |
| | chlorophenyl)-6-methyl-1,4-dihydropyrimidine- | | | | | |
| | 5-carboxamido)nicotinate | | | | | |
| 189 | 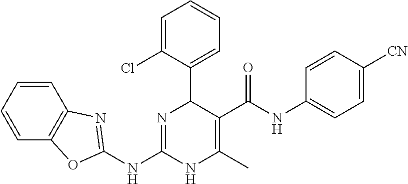 [SMILES: CC1=C(C(=O)Nc2ccc(C#N)cc2)C(c2ccccc2Cl)N=C(Nc2nc3ccccc3o2)N1] | 10.86 (−1.2) | 13.67 (−1.2) | 25.33109 | <18.7 | <1 |
| | 2-(benzo[d]oxazol-2-ylamino)-4-(2- | | | | | |
| | chlorophenyl)-N-(4-cyanophenyl)-6-methyl-1,4- | | | | | |
| | dihydropyrimidine-5-carboxamide | | | | | |
| 190 | 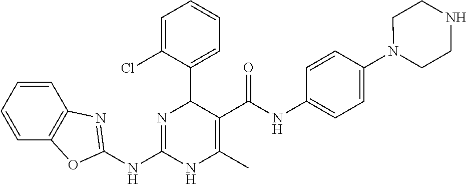 [SMILES: CC1=C(C(=O)Nc2ccc(N3CCNCC3)cc2)C(c2ccccc2Cl)N=C(Nc2nc3ccccc3o2)N1] | 68.53 (−2.2) | 48.51 (−2.2) | 8.63392 | 893.64587 | 2.41602 |
| | 2-(benzo[d]oxazol-2-ylamino)-4-(2- | | | | | |
| | chlorophenyl)-6-methyl-N-(4-(piperazin-1- | | | | | |
| | yl)phenyl)-1,4-dihydropyrimidine-5- | | | | | |
| | carboxamide | | | | | |
| 191 | 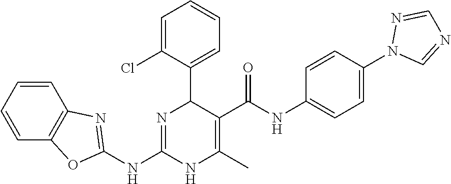 [SMILES: CC1=C(C(=O)Nc2ccc(-n3cncn3)cc2)C(c2ccccc2Cl)N=C(Nc2nc3ccccc3o2)N1] | 24.32 (−1.2) | 34.35 (−1.2) | na | na | na |
| | N-(4-(1H-1,2,4-triazol-1-yl)phenyl)-2- | | | | | |
| | (benzo[d]oxazol-2-ylamino)-4-(2-chlorophenyl)- | | | | | |
| | 6-methyl-1,4-dihydropyrimidine-5-carboxamide | | | | | |
| 192 | 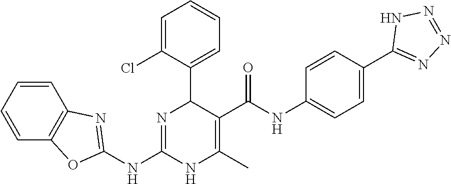 [SMILES: CC1=C(C(=O)Nc2ccc(-c3nnn[nH]3)cc2)C(c2ccccc2Cl)N=C(Nc2nc3ccccc3o2)N1] | 4.85 | na | 30 | <1.7 | 33.4 |
| | N-(4-(1H-tetrazol-5-yl)phenyl)-2- | | | | | |
| | (benzo[d]oxazol-2-ylamino)-4-(2-chlorophenyl)- | | | | | |
| | 6-methyl-1,4-dihydropyrimidine-5-carboxamide | | | | | |
| 193 | 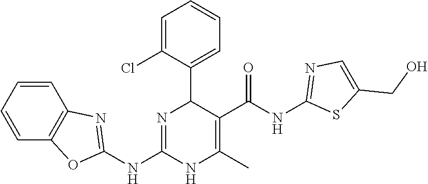 [SMILES: CC1=C(C(=O)Nc2ncc(CO)s2)C(c2ccccc2Cl)N=C(Nc2nc3ccccc3o2)N1] | 12.19 (−1.2) | 19.31 (−1.2) | >30 | 161.4 | <1 |
| | 2-(benzo[d]oxazol-2-ylamino)-4-(2- | | | | | |
| | chlorophenyl)-N-(5-(hydroxymethyl)thiazol-2- | | | | | |
| | yl)-6-methyl-1,4-dihydropyrimidine-5- | | | | | |
| | carboxamide | | | | | |
| 194 | 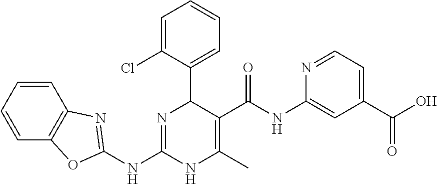 [SMILES: CC1=C(C(=O)Nc2cc(C(=O)O)ccn2)C(c2ccccc2Cl)N=C(Nc2nc3ccccc3o2)N1] | 1.08 | 6.85 | >30 | <1 | >74 |
| | 2-(2-(benzo[d]oxazol-2-ylamino)-4-(2- | | | | | |
| | chlorophenyl)-6-methyl-1,4-dihydropyrimidine- | | | | | |
| | 5-carboxamido)isonicotinic acid | | | | | |
| 195 | 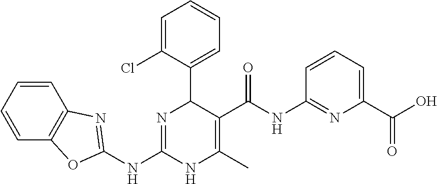 [SMILES: CC1=C(C(=O)Nc2cccc(C(=O)O)n2)C(c2ccccc2Cl)N=C(Nc2nc3ccccc3o2)N1] | 4.32 | 6.85 | >30 | <1 | 60.0 |
| | 6-(2-(benzo[d]oxazol-2-ylamino)-4-(2- | | | | | |
| | chlorophenyl)-6-methyl-1,4-dihydropyrimidine- | | | | | |
| | 5-carboxamido)picolinic acid | | | | | |
| 196 | 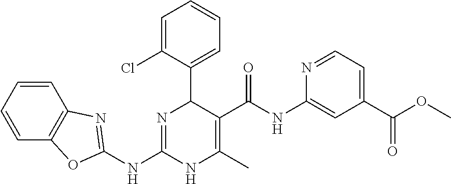 [SMILES: COC(=O)c1ccnc(NC(=O)C2=C(C)NC(Nc3nc4ccccc4o3)=NC2c2ccccc2Cl)c1] | 2.73 (−1.2) | 5.44 (−1.2) | 18.44471 | 27.7 | <1 |
| | methyl 2-(2-(benzo[d]oxazol-2-ylamino)-4-(2- | | | | | |
| | chlorophenyl)-6-methyl-1,4-dihydropyrimidine- | | | | | |
| | 5-carboxamido)isonicotinate | | | | | |
| 197 | 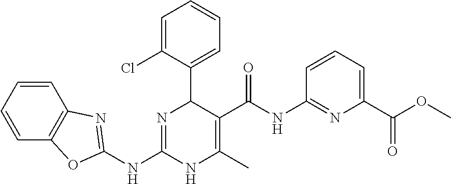 [SMILES: COC(=O)c1cccc(NC(=O)C2=C(C)NC(Nc3nc4ccccc4o3)=NC2c2ccccc2Cl)n1] | 9.68 (−1.2) | 12.19 (−1.2) | >30 | 18.9 | <1 |
| | methyl 2-(2-(benzo[d]oxazol-2-ylamino)-4-(2- | | | | | |
| | chlorophenyl)-6-methyl-1,4-dihydropyrimidine- | | | | | |
| | 5-carboxamido)isonicotinate | | | | | |
| 198 | 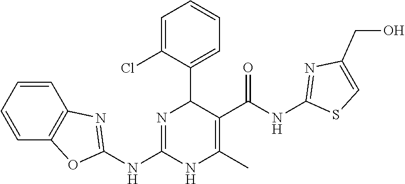 [SMILES: CC1=C(C(=O)Nc2nc(CO)cs2)C(c2ccccc2Cl)N=C(Nc2nc3ccccc3o2)N1] | 17.21 | 19.31 (−1.2) | >30 | 59.9 | <1 |
| | 2-(benzo[d]oxazol-2-ylamino)-4-(2- | | | | | |
| | chlorophenyl)-N-(4-(hydroxymethyl)thiazol-2- | | | | | |
| | yl)-6-methyl-1,4-dihydropyrimidine-5- | | | | | |
| | carboxamide | | | | | |
| 199 | 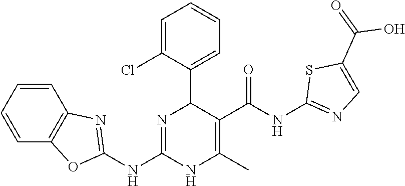 [SMILES: CC1=C(C(=O)Nc2ncc(C(=O)O)s2)C(c2ccccc2Cl)N=C(Nc2nc3ccccc3o2)N1] | 1.72 | na | >30 | <1 | >75 |
| | 2-(2-(benzo[d]oxazol-2-ylamino)-4-(2- | | | | | |
| | chlorophenyl)-6-methyl-1,4-dihydropyrimidine- | | | | | |
| | 5-carboxamido)thiazole-5-carboxylic acid | | | | | |
| 200 | 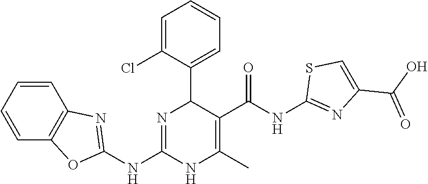 [SMILES: CC1=C(C(=O)Nc2nc(C(=O)O)cs2)C(c2ccccc2Cl)N=C(Nc2nc3ccccc3o2)N1] | 9.68 | 19.31 (−1.2) | na | na | na |
| | 2-(2-(benzo[d]oxazol-2-ylamino)-4-(2- | | | | | |
| | chlorophenyl)-6-methyl-1,4-dihydropyrimidine- | | | | | |
| | 5-carboxamido)thiazole-4-carboxylic acid | | | | | |
| 201 | 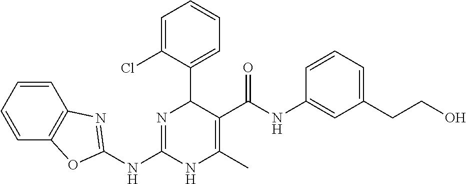 [SMILES: CC1=C(C(=O)Nc2cccc(CCO)c2)C(c2ccccc2Cl)N=C(Nc2nc3ccccc3o2)N1] | 17.21 (−2.2) | na | 26.59477 | na | na |
| | 2-(benzo[d]oxazol-2-ylamino)-4-(2- | | | | | |
| | chlorophenyl)-N-(3-(2-hydroxyethyl)phenyl)-6- | | | | | |
| | methyl-1,4-dihydropyrimidine-5-carboxamide | | | | | |
| 202 | 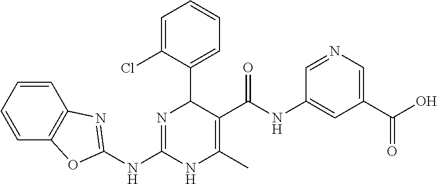 [SMILES: CC1=C(C(=O)Nc2cncc(C(=O)O)c2)C(c2ccccc2Cl)N=C(Nc2nc3ccccc3o2)N1] | 4.32 | na | >30 | na | 60.3 |
| | 5-(2-(benzo[d]oxazol-2-ylamino)-4-(2- | | | | | |
| | chlorophenyl)-6-methyl-1,4-dihydropyrimidine- | | | | | |
| | 5-carboxamido)nicotinic acid | | | | | |
| 203 | 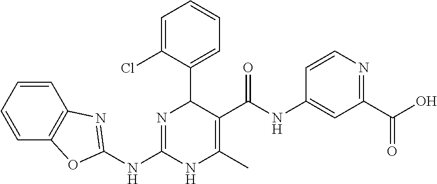 [SMILES: CC1=C(C(=O)Nc2ccnc(C(=O)O)c2)C(c2ccccc2Cl)N=C(Nc2nc3ccccc3o2)N1] | 3.43 | 3.06 | >30 | <1 | >74 |
| | 4-(2-(benzo[d]oxazol-2-ylamino)-4-(2- | | | | | |
| | chlorophenyl)-6-methyl-1,4-dihydropyrimidine- | | | | | |
| | 5-carboxamido)picolinic acid | | | | | |
| 204 | 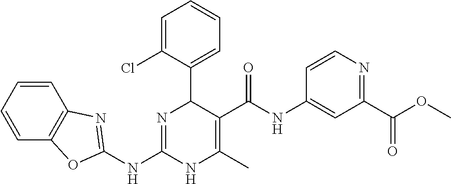 [SMILES: COC(=O)c1cc(NC(=O)C2=C(C)NC(Nc3nc4ccccc4o3)=NC2c2ccccc2Cl)ccn1] | 7.69 | 10.86 | >30 | 49.2 | <1 |
| | methyl 4-(2-(benzo[d]oxazol-2-ylamino)-4-(2- | | | | | |
| | chlorophenyl)-6-methyl-1,4-dihydropyrimidine- | | | | | |
| | 5-carboxamido)picolinate | | | | | |
| 205 | 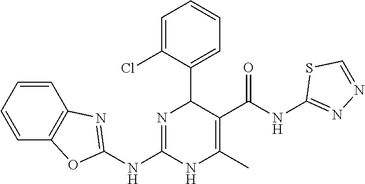 [SMILES: CC1=C(C(=O)Nc2nncs2)C(c2ccccc2Cl)N=C(Nc2nc3ccccc3o2)N1] | 3.85 | 7.69 | >30 | 207.3 | <1 |
| | 2-(benzo[d]oxazol-2-ylamino)-4-(2- | | | | | |
| | chlorophenyl)-6-methyl-N-(1,3,4-thiadiazol-2- | | | | | |
| | yl)-1,4-dihydropyrimidine-5-carboxamide | | | | | |
| 206 | 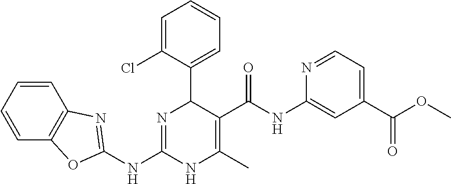 [SMILES: COC(=O)c1ccnc(NC(=O)C2=C(C)NC(Nc3nc4ccccc4o3)=NC2c2ccccc2Cl)c1] | 3.43 (−1.2) | 6.85 (−1.2) | 19.98873 | 306.7 | <1 |
| | (R)-methyl 2-(2-(benzo[d]oxazol-2-ylamino)-4- | | | | | |
| | (2-chlorophenyl)-6-methyl-1,4- | | | | | |
| | dihydropyrimidine-5-carboxamido)isonicotinate | | | | | |
| 207 | 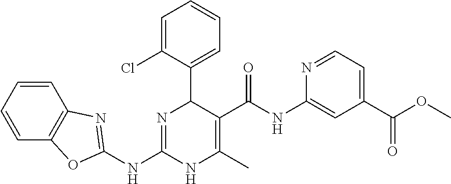 [SMILES: COC(=O)c1ccnc(NC(=O)C2=C(C)NC(Nc3nc4ccccc4o3)=NC2c2ccccc2Cl)c1] | in- active | in- active | 25.55465 | 250.4 | <1 |
| | (S)-methyl 2-(2-(benzo[d]oxazol-2-ylamino)-4- | | | | | |
| | (2-chlorophenyl)-6-methyl-1,4- | | | | | |
| | dihydropyrimidine-5-carboxamido)isonicotinate | | | | | |
| 208 | 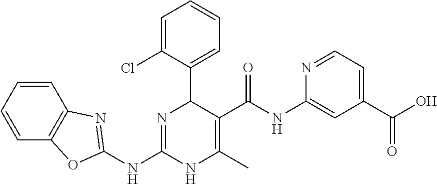 [SMILES: CC1=C(C(=O)Nc2cc(C(=O)O)ccn2)C(c2ccccc2Cl)N=C(Nc2nc3ccccc3o2)N1] | 0.54 | 3.43 | >30 | <1 | >74 |
| | (R)-2-(2-(benzo[d]oxazol-2-ylamino)-4-(2- | | | | | |
| | chlorophenyl)-6-methyl-1,4-dihydropyrimidine- | | | | | |
| | 5-carboxamido)isonicotinic acid | | | | | |
| 209 | 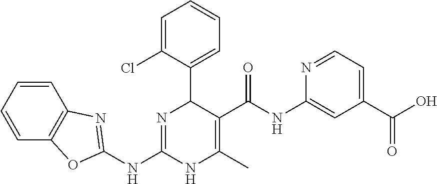 [SMILES: CC1=C(C(=O)Nc2cc(C(=O)O)ccn2)C(c2ccccc2Cl)N=C(Nc2nc3ccccc3o2)N1] | 12.19 | 19.31 (−1.2) | >30 | <1 | >74 |
| | (S)-2-(2-(benzo[d]oxazol-2-ylamino)-4-(2- | | | | | |
| | chlorophenyl)-6-methyl-1,4-dihydropyrimidine- | | | | | |
| | 5-carboxamido)isonicotinic acid | | | | | |
| 210 | 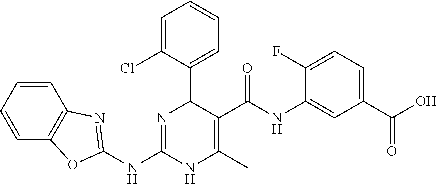 [SMILES: CC1=C(C(=O)Nc2cc(C(=O)O)ccc2F)C(c2ccccc2Cl)N=C(Nc2nc3ccccc3o2)N1] | 3.06 | 10.86 | >30 | 3.3 | 25.4 |
| | 3-(2-(benzo[d]oxazol-2-ylamino)-4-(2- | | | | | |
| | chlorophenyl)-6-methyl-1,4-dihydropyrimidine- | | | | | |
| | 5-carboxamido)-4-fluorobenzoic acid | | | | | |
| 211 | 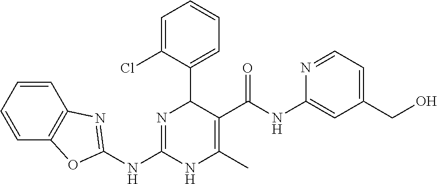 [SMILES: CC1=C(C(=O)Nc2cc(CO)ccn2)C(c2ccccc2Cl)N=C(Nc2nc3ccccc3o2)N1] | 3.06 | 9.68 | >30 | 109.6 | <1 |
| | 2-(benzo[d]oxazol-2-ylamino)-4-(2- | | | | | |
| | chlorophenyl)-N-(4-(hydroxymethyl)pyridin-2- | | | | | |
| | yl)-6-methyl-1,4-dihydropyrimidine-5- | | | | | |
| | carboxamide | | | | | |
| 212 | 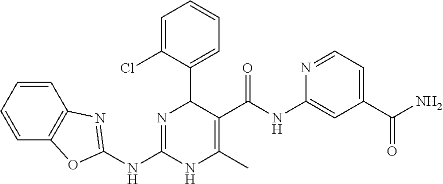 [SMILES: CC1=C(C(=O)Nc2cc(C(N)=O)ccn2)C(c2ccccc2Cl)N=C(Nc2nc3ccccc3o2)N1] | 2.43 | 7.69 | >30 | 156.0 | <1 |
| | 2-(benzo[d]oxazol-2-ylamino)-N-(4- | | | | | |
| | carbamoylpyridin-2-yl)-4-(2-chlorophenyl)-6- | | | | | |
| | methyl-1,4-dihydropyrimidine-5-carboxamide | | | | | |
| 213 | 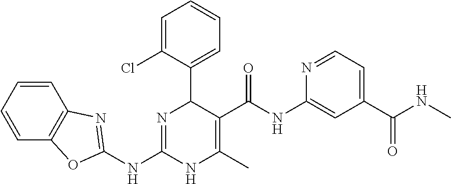 [SMILES: CNC(=O)c1ccnc(NC(=O)C2=C(C)NC(Nc3nc4ccccc4o3)=NC2c2ccccc2Cl)c1] | 3.06 | 10.86 | >30 | 421.9 | <1 |
| | 2-(benzo[d]oxazol-2-ylamino)-4-(2- | | | | | |
| | chlorophenyl)-6-methyl-N-(4- | | | | | |
| | (methylcarbamoyl)pyridin-2-yl)-1,4- | | | | | |
| | dihydropyrimidine-5-carboxamide | | | | | |
| 214 | 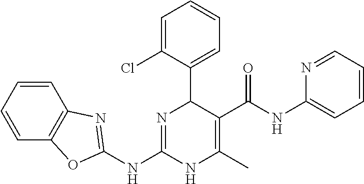 [SMILES: CC1=C(C(=O)Nc2ccccn2)C(c2ccccc2Cl)N=C(Nc2nc3ccccc3o2)N1] | 4.32 (−1.2) | 10.86 (−1.2) | >30 | 173.5 | <1 |
| | 2-(benzo[d]oxazol-2-ylamino)-4-(2- | | | | | |
| | chlorophenyl)-6-methyl-N-(pyridin-2-yl)-1,4- | | | | | |
| | dihydropyrimidine-5-carboxamide | | | | | |
| 215 | 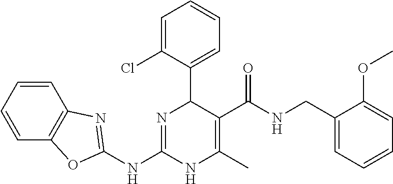 [SMILES: COc1ccccc1CNC(=O)C1=C(C)NC(Nc2nc3ccccc3o2)=NC1c1ccccc1Cl] | 7.69 (−1.2) | 12.19 (−1.2) | 1.4 | 99.3 | <1 |
| | 2-(benzo[d]oxazol-2-ylamino)-4-(2- | | | | | |
| | chlorophenyl)-N-(2-methoxybenzyl)-6-methyl- | | | | | |
| | 1,4-dihydropyrimidine-5-carboxamide | | | | | |
| 216 | 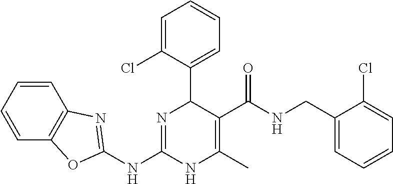 [SMILES: CC1=C(C(=O)NCc2ccccc2Cl)C(c2ccccc2Cl)N=C(Nc2nc3ccccc3o2)N1] | in- active | in- active | 4.22324 | na | <1 |
| | 2-(benzo[d]oxazol-2-ylamino)-N-(2- | | | | | |
| | chlorobenzyl)-4-(2-chlorophenyl)-6-methyl-1,4- | | | | | |
| | dihydropyrimidine-5-carboxamide | | | | | |
| 217 | 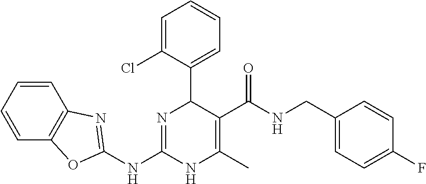 [SMILES: CC1=C(C(=O)NCc2ccc(F)cc2)C(c2ccccc2Cl)N=C(Nc2nc3ccccc3o2)N1] | 7.69 (−1.2) | in- active | 11.34137 | na | <1 |
| | 2-(benzo[d]oxazol-2-ylamino)-4-(2- | | | | | |
| | chlorophenyl)-N-(4-fluorophenyl)-6-methyl-1,4- | | | | | |
| | dihydropyrimidine-5-carboxamide | | | | | |
| 218 | 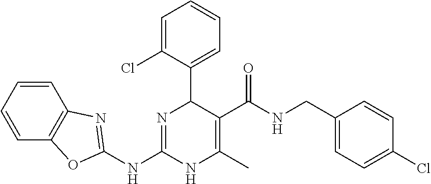 [SMILES: CC1=C(C(=O)NCc2ccc(Cl)cc2)C(c2ccccc2Cl)N=C(Nc2nc3ccccc3o2)N1] | in- active | in- active | 11.90678 | 25.8 | <1 |
| | 2-(benzo[d]oxazol-2-ylamino)-4-(2- | | | | | |
| | chlorophenyl)-N-(4-chlorobenzyl)-6-methyl-1,4- | | | | | |
| | dihydropyrimidine-5-carboxamide | | | | | |
| 219 | 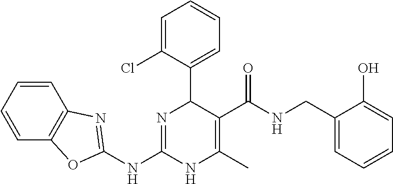 [SMILES: CC1=C(C(=O)NCc2ccccc2O)C(c2ccccc2Cl)N=C(Nc2nc3ccccc3o2)N1] | 6.85 (−1.2) | 10.86 (−1.2) | 5.18837 | <19.3 | na |
| | 2-(benzo[d]oxazol-2-ylamino)-4-(2- | | | | | |
| | chlorophenyl)-N-(2-hydroxybenzyl)-6-methyl- | | | | | |
| | 1,4-dihydropyrimidine-5-carboxamide | | | | | |
| 220 | 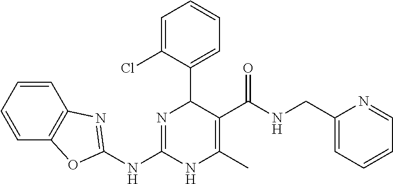 [SMILES: CC1=C(C(=O)NCc2ccccn2)C(c2ccccc2Cl)N=C(Nc2nc3ccccc3o2)N1] | 8.63 | 19.31 (−1.2) | 6.55741 | 1424.0 | 1.8 |
| | 2-(benzo[d]oxazol-2-ylamino)-4-(2- | | | | | |
| | chlorophenyl)-6-methyl-N-(pyridin-2-ylmethyl)- | | | | | |
| | 1,4-dihydropyrimidine-5-carboxamide | | | | | |
| 221 | 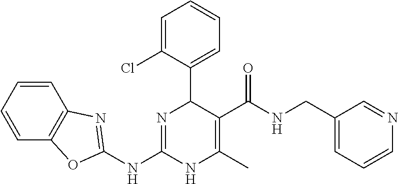 [SMILES: CC1=C(C(=O)NCc2cccnc2)C(c2ccccc2Cl)N=C(Nc2nc3ccccc3o2)N1] | 6.85 | 19.31 (−1.2) | 1.58187 | 1248.8 | <1 |
| | 2-(benzo[d]oxazol-2-ylamino)-4-(2- | | | | | |
| | chlorophenyl)-6-methyl-N-(pyridin-3-ylmethyl)- | | | | | |
| | 1,4-dihydropyrimidine-5-carboxamide | | | | | |
| 222 | 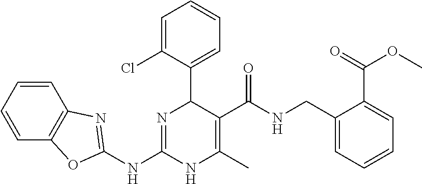 [SMILES: COC(=O)c1ccccc1CNC(=O)C1=C(C)NC(Nc2nc3ccccc3o2)=NC1c1ccccc1Cl] | 6.85 (−1.2) | 4.32 (−1.2) | 1.74249 | 462.5 | <1 |
| | methyl 2-((2-(benzo[d]oxazol-2-ylamino)-4-(2- | | | | | |
| | chlorophenyl)-6-methyl-1,4-dihydropyrimidine- | | | | | |
| | 5-carboxamido)methyl)benzoate | | | | | |
| 223 | 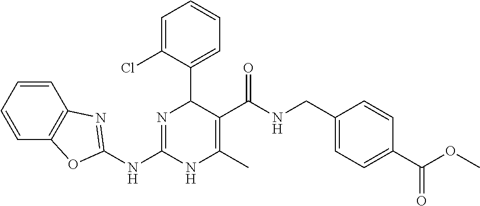 [SMILES: COC(=O)c1ccc(CNC(=O)C2=C(C)NC(Nc3nc4ccccc4o3)=NC2c2ccccc2Cl)cc1] | 4.32 (−1.2) | 8.63 (−1.2) | 8.49452 | 543.4 | <1 |
| | methyl 4-((2-(benzo[d]oxazol-2-ylamino)-4-(2- | | | | | |
| | chlorophenyl)-6-methyl-1,4-dihydropyrimidine- | | | | | |
| | 5-carboxamido)methyl)benzoate | | | | | |
| 224 | 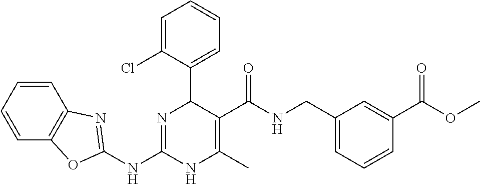 [SMILES: COC(=O)c1cccc(CNC(=O)C2=C(C)NC(Nc3nc4ccccc4o3)=NC2c2ccccc2Cl)c1] | 4.85 (−1.4) | 7.69 (−1.2) | 3.41557 | 615.7 | <1 |
| | methyl 3-((2-(benzo[d]oxazol-2-ylamino)-4-(2- | | | | | |
| | chlorophenyl)-6-methyl-1,4-dihydropyrimidine- | | | | | |
| | 5-carboxamido)methyl)benzoate | | | | | |
| 225 | 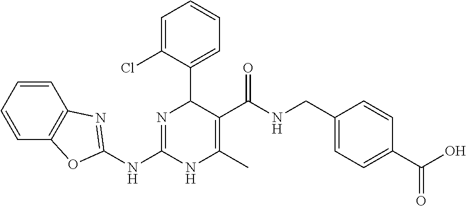 [SMILES: CC1=C(C(=O)NCc2ccc(C(=O)O)cc2)C(c2ccccc2Cl)N=C(Nc2nc3ccccc3o2)N1] | 3.43 | 10.86 (−1.2) | >30 | <1 | 47.0 |
| | 4-((2-(benzo[d]oxazol-2-ylamino)-4-(2- | | | | | |
| | chlorophenyl)-6-methyl-1,4-dihydropyrimidine- | | | | | |
| | 5-carboxamido)methyl)benzoic acid | | | | | |
| 226 | 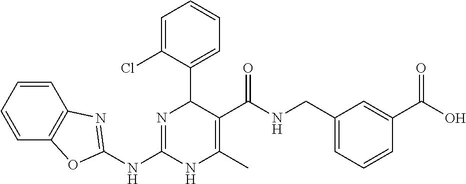 [SMILES: CC1=C(C(=O)NCc2cccc(C(=O)O)c2)C(c2ccccc2Cl)N=C(Nc2nc3ccccc3o2)N1] | 7.69 | 19.31 (−1.2) | >30 | <1.3 | 60.6 |
| | 3-((2-(benzo[d]oxazol-2-ylamino)-4-(2- | | | | | |
| | chlorophenyl)-6-methyl-1,4-dihydropyrimidine- | | | | | |
| | 5-carboxamido)methyl)benzoic acid | | | | | |
| 227 | 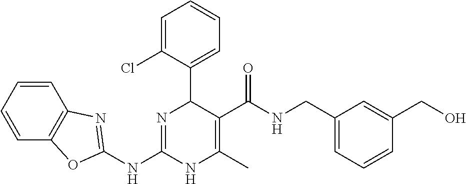 [SMILES: CC1=C(C(=O)NCc2cccc(CO)c2)C(c2ccccc2Cl)N=C(Nc2nc3ccccc3o2)N1] | 4.85 | 12.19 | 6.57359 | 868.8 | <1 |
| | 2-(benzo[d]oxazol-2-ylamino)-4-(2- | | | | | |
| | chlorophenyl)-N-(3-(hydroxymethyl)benzyl)-6- | | | | | |
| | methyl-1,4-dihydropyrimidine-5-carboxamide | | | | | |
| 228 | 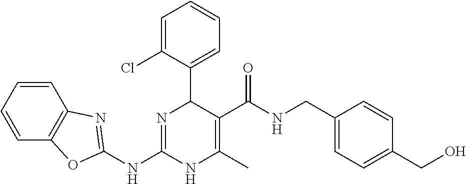 [SMILES: CC1=C(C(=O)NCc2ccc(CO)cc2)C(c2ccccc2Cl)N=C(Nc2nc3ccccc3o2)N1] | 6.85 | 15.34 (−1.2) | 13.8241 | 867.3 | <1 |
| | 2-(benzo[d]oxazol-2-ylamino)-4-(2- | | | | | |
| | chlorophenyl)-N-(4-(hydroxymethyl)benzyl)-6- | | | | | |
| | methyl-1,4-dihydropyrimidine-5-carboxamide | | | | | |
| 229 | 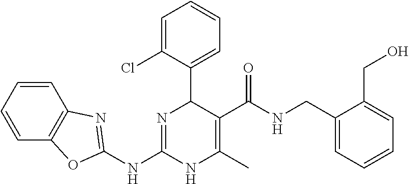 [SMILES: CC1=C(C(=O)NCc2ccccc2CO)C(c2ccccc2Cl)N=C(Nc2nc3ccccc3o2)N1] | 6.11 | 15.34 (−1.2) | 5.0859 | 1362.3 | <1 |
| | 2-(benzo[d]oxazol-2-ylamino)-4-(2- | | | | | |
| | chlorophenyl)-N-(2-(hydroxymethyl)benzyl)-6- | | | | | |
| | methyl-1,4-dihydropyrimidine-5-carboxamide | | | | | |
| 230 | 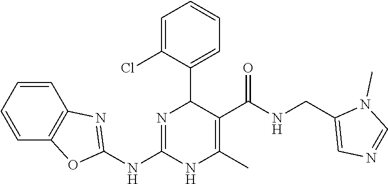 [SMILES: CC1=C(C(=O)NCc2cncn2C)C(c2ccccc2Cl)N=C(Nc2nc3ccccc3o2)N1] | 1.72 | 6.85 (−1.2) | 8.8722 | 98.8 | 42.0 |
| | 2-(benzo[d]oxazol-2-ylamino)-4-(2- | | | | | |
| | chlorophenyl)-6-methyl-N-((1-methyl-1H- | | | | | |
| | imidazol-5-yl)methyl)-1,4-dihydropyrimidine-5- | | | | | |
| | carboxamide | | | | | |
| 231 | 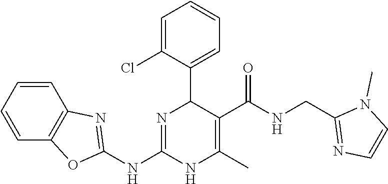 [SMILES: CC1=C(C(=O)NCc2nccn2C)C(c2ccccc2Cl)N=C(Nc2nc3ccccc3o2)N1] | 15.34 | 13.67 | 6.41517 | >1113 | 23.0 |
| | 2-(benzo[d]oxazol-2-ylamino)-4-(2- | | | | | |
| | chlorophenyl)-6-methyl-N-((1-methyl-1H- | | | | | |
| | imidazol-2-ylmethyl)-1,4-dihydropyrimidine-5- | | | | | |
| | carboxamide | | | | | |
| 232 | 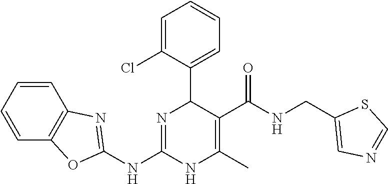 [SMILES: CC1=C(C(=O)NCc2cncs2)C(c2ccccc2Cl)N=C(Nc2nc3ccccc3o2)N1] | 1.93 | 12.19 (−1.2) | 6.43209 | 507.4 | <1 |
| | 2-(benzo[d]oxazol-2-ylamino)-4-(2- | | | | | |
| | chlorophenyl)-6-methyl-N-(thiazol-5-ylmethyl)- | | | | | |
| | 1,4-dihydropyrimidine-5-carboxamide | | | | | |
| 233 | 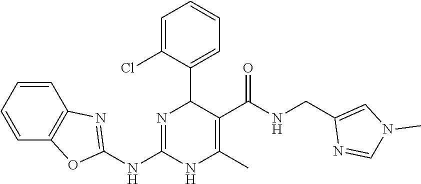 [SMILES: CC1=C(C(=O)NCc2cn(C)cn2)C(c2ccccc2Cl)N=C(Nc2nc3ccccc3o2)N1] | 1.09 | 7.69 | 14.55623 | 868.4 | 26.6 |
| | 2-(benzo[d]oxazol-2-ylamino)-4-(2- | | | | | |
| | chlorophenyl)-6-methyl-N-((1-methyl-1H- | | | | | |
| | imidazol-4-yl)methyl)-1,4-dihydropyrimidine-5- | | | | | |
| | carboxamide | | | | | |
| 234 | 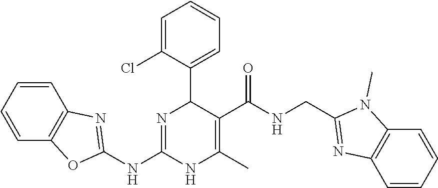 [SMILES: CC1=C(C(=O)NCc2nc3ccccc3n2C)C(c2ccccc2Cl)N=C(Nc2nc3ccccc3o2)N1] | 8.63 (−1.2) | 13.67 (−1.2) | 4.51623 | na | <1 |
| | 2-(benzo[d]oxazol-2-ylamino)-4-(2- | | | | | |
| | chlorophenyl)-6-methyl-N-((1-methyl-1H- | | | | | |
| | benzo[d]imidazol-2-yl)methyl)-1,4- | | | | | |
| | dihydropyrimidine-5-carboxamide | | | | | |
| 235 | 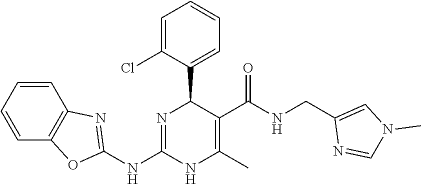 [SMILES: CC1=C(C(=O)NCc2cn(C)cn2)[C@H](c2ccccc2Cl)N=C(Nc2nc3ccccc3o2)N1] | 3.06 | 4.32 | 5.62059 | 866.5 | 46.1 |
| | (R)-2-(benzo[d]oxazol-2-ylamino)-4-(2- | | | | | |
| | chlorophenyl)-6-methyl-N-((1-methyl-1H- | | | | | |
| | imidazol-4-yl)methyl)-1,4-dihydropyrimidine-5- | | | | | |
| | carboxamide | | | | | |
| 236 | 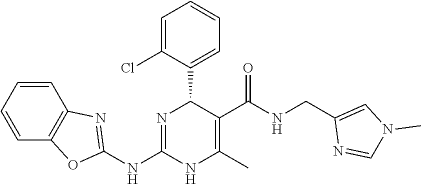 [SMILES: CC1=C(C(=O)NCc2cn(C)cn2)[C@@H](c2ccccc2Cl)N=C(Nc2nc3ccccc3o2)N1] | in- active | in- active | 8.72597 | | 40.5 |
| | (S)-2-(benzo[d]oxazol-2-ylamino)-4-(2- | | | | | |
| | chlorophenyl)-6-methyl-N-((1-methyl-1H- | | | | | |
| | imidazol-4-yl)methyl)-1,4-dihydropyrimidine-5- | | | | | |
| | carboxamide | | | | | |
| 237 | 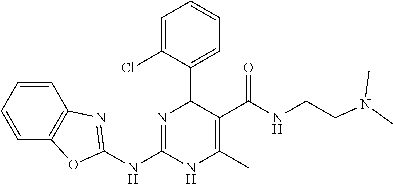 [SMILES: CC1=C(C(=O)NCCN(C)C)C(c2ccccc2Cl)N=C(Nc2nc3ccccc3o2)N1] | 19.31 (−1.2) | 15.34 (−1.2) | >30 | >1227 | >67 |
| | 2-(benzo[d]oxazol-2-ylamino)-4-(2- | | | | | |
| | chlorophenyl)-N-(2-dimethylamino)ethyl)-6- | | | | | |
| | methyl-1,4-dihydropyrimidine-5-carboxamide | | | | | |
| 238 | 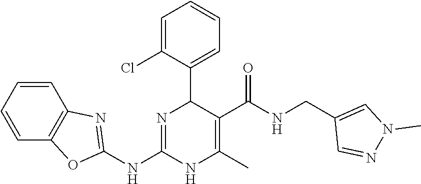 [SMILES: CC1=C(C(=O)NCc2cnn(C)c2)C(c2ccccc2Cl)N=C(Nc2nc3ccccc3o2)N1] | 1.37 | 12.19 | 15.66747 | 807.7 | 24.2 |
| | 2-(benzo[d]oxazol-2-ylamino)-4-(2- | | | | | |
| | chlorophenyl)-6-methyl-N-((1-methyl-1H- | | | | | |
| | pyrazol-4-yl)methyl)-1,4-dihydropyrimidine-5- | | | | | |
| | carboxamide | | | | | |
| 239 | 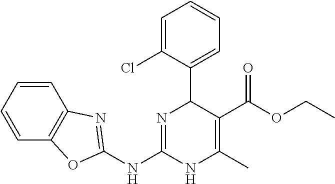 [SMILES: CCOC(=O)C1=C(C)NC(Nc2nc3ccccc3o2)=NC1c1ccccc1Cl] | in- active | 13.67 (−1.2) | 19.30 | na | <1 |
| | ethyl 2-(benzo[d]oxazol-2-ylamino)-4-(2- | | | | | |
| | chlorophenyl)-6-methyl-1,4-dihydropyrimidine- | | | | | |
| | 5-carboxylate | | | | | |
| 240 | 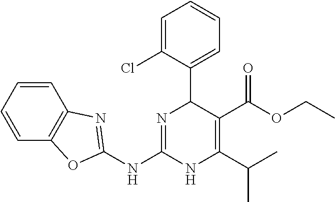 [SMILES: CCOC(=O)C1=C(C(C)C)NC(Nc2nc3ccccc3o2)=NC1c1ccccc1Cl] | in- active | in- active | 57.5 | na | <1 |
| | ethyl 2-(benzo[d]oxazol-2-ylamino)-4-(2- | | | | | |
| | chlorophenyl)-6-isopropyl-1,4- | | | | | |
| | dihydropyrimidine-5-carboxylate | | | | | |
| 241 | 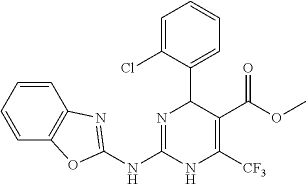 [SMILES: COC(=O)C1=C(C(F)(F)F)NC(Nc2nc3ccccc3o2)=NC1c1ccccc1Cl] | in- active | in- active | 10.46 | na | 2.65 |
| | methyl 2-(benzo[d]oxazol-2-ylamino)-4-(2- | | | | | |
| | chlorophenyl)-6-(trifluoromethyl)-1,4- | | | | | |
| | dihydropyrimidine-5-carboxylate | | | | | |
| 242 | 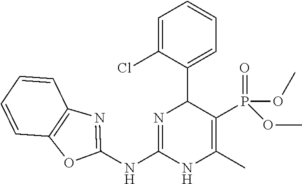 [SMILES: COP(=O)(OC)C1=C(C)NC(Nc2nc3ccccc3o2)=NC1c1ccccc1Cl] | 17.21 (−1.2) | 13.67 (−1.2) | 5.35 | 1813.86 | 12.21 |
| | dimethyl (2-(benzo[d]oxazol-2-ylamino)-4-(2- | | | | | |
| | chlorophenyl)-6-methyl-1,4-dihydropyrimidin-5- | | | | | |
| | yl)phosphonate | | | | | |
| 243 | 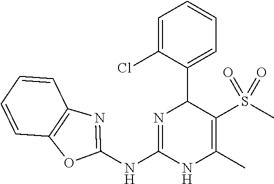 [SMILES: CC1=C(S(C)(=O)=O)C(c2ccccc2Cl)N=C(Nc2nc3ccccc3o2)N1] | 13.67 (−1.2) | 19.31 (−1.2) | 9.37 | 42.78 | <1 |
| | N-(4-(2-chlorophenyl)-6-methyl-5- | | | | | |
| | (methylsulfonyl)-1,4-dihydropyrimidin-2- | | | | | |
| | yl)benzo[d]oxazol-2-amine | | | | | |
| 244 | 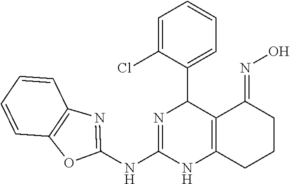 [SMILES: O/N=C1\CCCC2=C1C(c1ccccc1Cl)N=C(Nc1nc3ccccc3o1)N2] | 17.21 (−1.2) | 10.86 | 5.48 | 244.29 | <1 |
| | (E)-2-(benzo[d]oxazol-2-ylamino)-4-(2- | | | | | |
| | chlorophenyl)-4,6,7,8-tetrahydroquinazolin- | | | | | |
| | 5(1H)-one oxime | | | | | |
| 245 | 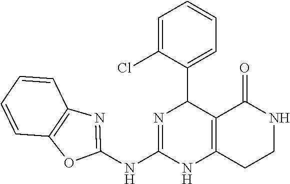 [SMILES: O=C1NCCC2=C1C(c1ccccc1Cl)N=C(Nc1nc3ccccc3o1)N2] | 17.21 (−1.2) | 21.67 (−1.2) | 27.12 | na | <1 |
| | 2-(benzo[d]oxazol-2-ylamino)-4-(2- | | | | | |
| | chlorophenyl)-4,6,7,8-tetrahydropyrido[4,3- | | | | | |
| | d]pyrimidin-5(1H)-one | | | | | |
| 246 | 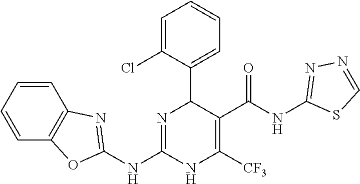 [SMILES: O=C(Nc1nncs1)C1=C(C(F)(F)F)NC(Nc2nc3ccccc3o2)=NC1c1ccccc1Cl] | 13.67 | 17.21 (−1.2) | >30 | 715.1 | 21.9 |
| | 2-(benzo[d]oxazol-2-ylamino)-4-(2- | | | | | |
| | chlorophenyl)-N-(1,3,4-thiadiazol-2-yl)-6- | | | | | |
| | (trifluoromethyl)-1,4-dihydropyrimidine-5- | | | | | |
| | carboxamide | | | | | |
| 247 | 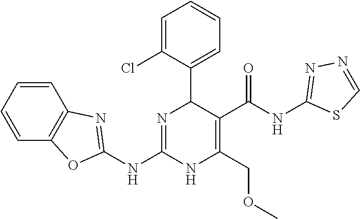 [SMILES: COCC1=C(C(=O)Nc2nncs2)C(c2ccccc2Cl)N=C(Nc2nc3ccccc3o2)N1] | 13.67 | 19.31 (−1.2) | >30 | 319.8 | <1 |
| | 2-(benzo[d]oxazol-2-ylamino)-4-(2- | | | | | |
| | chlorophenyl)-6-(methoxyphenyl)-N-(1,3,4- | | | | | |
| | thiadiazol-2-yl)-1,4-dihydropyrimidine-5- | | | | | |
| | carboxamide | | | | | |
| 248 | 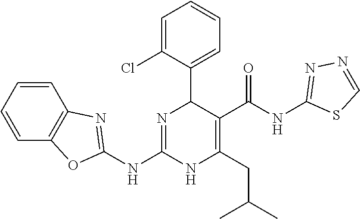 [SMILES: CC(C)CC1=C(C(=O)Nc2nncs2)C(c2ccccc2Cl)N=C(Nc2nc3ccccc3o2)N1] | 5.44 (−1.2) | 17.21 (−2.2) | >30 | 42.7 | <1 |
| | 2-(benzo[d]oxazol-2-ylamino)-4-(2- | | | | | |
| | chlorophenyl)-6-isobutyl-N-(1,3,4-thiadiazol-2- | | | | | |
| | yl)-1,4-dihydropyrimidine-5-carboxamide | | | | | |
| 249 | 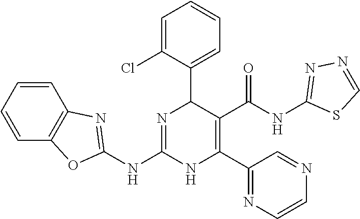 [SMILES: O=C(Nc1nncs1)C1=C(c2cnccn2)NC(Nc2nc3ccccc3o2)=NC1c1ccccc1Cl] | 3.85 | 21.67 (−1.2) | >30 | 60.3 | 2.1 |
| | 2-(benzo[d]oxazol-2-ylamino)-4-(2- | | | | | |
| | chlorophenyl)-6-(pyrazin-2-yl)-N-(1,3,4- | | | | | |
| | thiadiazol-2-yl)-1,4-dihydropyrimidine-5- | | | | | |
| | carboxamide | | | | | |
| 250 | 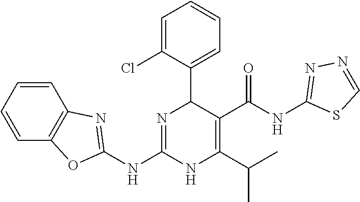 [SMILES: CC(C)C1=C(C(=O)Nc2nncs2)C(c2ccccc2Cl)N=C(Nc2nc3ccccc3o2)N1] | 7.69 (−1.2) | 15.34 (−1.2) | >30 | 24.6 | <1 |
| | 2-(benzo[d]oxazol-2-ylamino)-4-(2- | | | | | |
| | chlorophenyl)-6-isopropyl-N-(1,3,4-thiadiazol-2- | | | | | |
| | yl)-1,4-dihydropyrimidine-5-carboxamide | | | | | |
| 251 | 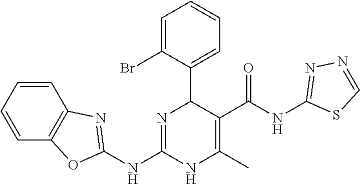 [SMILES: CC1=C(C(=O)Nc2nncs2)C(c2ccccc2Br)N=C(Nc2nc3ccccc3o2)N1] | 4.32 | 9.68 (−1.2) | >30 | 446.9 | <1 |
| | 2-(benzo[d]oxazol-2-ylamino)-4-(2- | | | | | |
| | bromophenyl)-6-methyl-N-(1,3,4-thiadiazol-2- | | | | | |
| | yl)-1,4-dihydropyrimidine-5-carboxamide | | | | | |
| 252 | 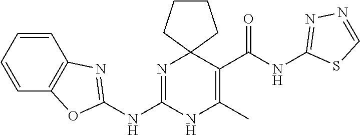 [SMILES: CC1=C(C(=O)Nc2nncs2)C2(CCCC2)N=C(Nc2nc3ccccc3o2)N1] | 3.85 | 19.31 (−1.2) | >30 | <1 | <1 |
| | 7-(benzo[d]oxazol-2-ylamino)-9-methyl-N- | | | | | |
| | (1,3,4-thiadiazol-2-yl)-6,8-diazaspiro[4.5]deca- | | | | | |
| | 6,9-diene-10-carboxamide | | | | | |
| 253 | 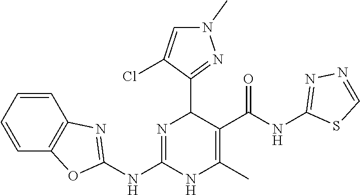 [SMILES: CC1=C(C(=O)Nc2nncs2)C(c2nn(C)cc2Cl)N=C(Nc2nc3ccccc3o2)N1] | 7.69 | 17.21 (−1.2) | >30 | 746.9 | 12.8 |
| | 2-(benzo[d]oxazol-2-ylamino)-4-(4-chloro-1- | | | | | |
| | methyl-1H-pyrazol-3-yl)-6-methyl-N-(1,3,4- | | | | | |
| | thiadiazol-2-yl)-1,4-dihydropyrimidine-5- | | | | | |
| | carboxamide | | | | | |
| 254 | 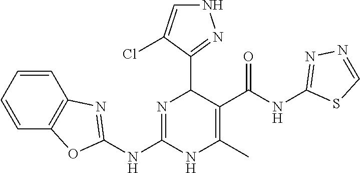 [SMILES: CC1=C(C(=O)Nc2nncs2)C(c2n[nH]cc2Cl)N=C(Nc2nc3ccccc3o2)N1] | 17.21 (−1.2) | 54.44 (−2.4) | >30 | 37.0 | 34.6 |
| | 2-(benzo[d]oxazol-2-ylamino)-4-(4-chloro-1H- | | | | | |
| | pyrazol-3-yl)-6-methyl-N-(1,3,4-thiadiazol-2-yl)- | | | | | |
| | 1,4-dihydropyrimidine-5-carboxamide | | | | | |
| 255 | 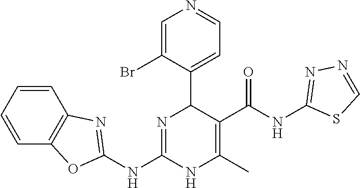 [SMILES: CC1=C(C(=O)Nc2nncs2)C(c2ccncc2Br)N=C(Nc2nc3ccccc3o2)N1] | 3.43 | 19.31 | >30 | 208.7 | 6.5 |
| | 2-(benzo[d]oxazol-2-ylamino)-4-(3- | | | | | |
| | bromopyridin-4-yl)-6-methyl-N-(1,3,4- | | | | | |
| | thiadiazol-2-yl)-1,4-dihydropyrimidine-5- | | | | | |
| | carboxamide | | | | | |
| 256 | 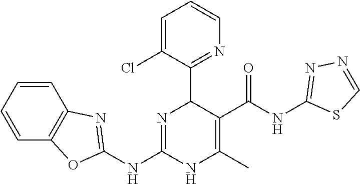 [SMILES: CC1=C(C(=O)Nc2nncs2)C(c2ncccc2Cl)N=C(Nc2nc3ccccc3o2)N1] | 6.85 | 19.31 (−1.2) | >30 | 695.3 | 10.6 |
| | 2-(benzo[d]oxazol-2-ylamino)-4-(3- | | | | | |
| | chloropyridin-2-yl)-6-methyl-N-(1,3,4-thiadiazol- | | | | | |
| | 2-yl)-1,4-dihydropyrimidine-5-carboxamide | | | | | |
| 257 | 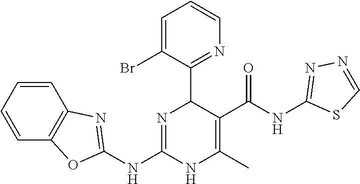 [SMILES: CC1=C(C(=O)Nc2nncs2)C(c2ncccc2Br)N=C(Nc2nc3ccccc3o2)N1] | 4.85 | 6.85 | >30 | 709.3 | 3.0 |
| | 2-(benzo[d]oxazol-2-ylamino)-4-(3- | | | | | |
| | bromopyridin-2-yl)-6-methyl-N-(1,3,4- | | | | | |
| | thiadiazol-2-yl)-1,4-dihydropyrimidine-5- | | | | | |
| | carboxamide | | | | | |
| 258 | 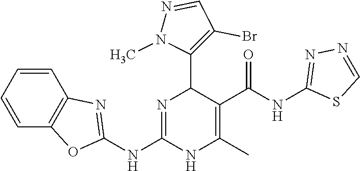 [SMILES: CC1=C(C(=O)Nc2nncs2)C(c2c(Br)cnn2C)N=C(Nc2nc3ccccc3o2)N1] | 21.67 (−1.2) | in- active | >30 | 198.3 | 38.7 |
| | 2-(benzo[d]oxazol-2-ylamino)-4-(4-bromo-1- | | | | | |
| | methyl-1H-pyrazol-5-yl)-6-methyl-N-(1,3,4- | | | | | |
| | thiadiazol-2-yl)-1,4-dihydropyrimidine-5- | | | | | |
| | carboxamide | | | | | |
| 259 | 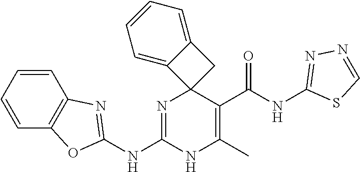 [SMILES: CC1=C(C(=O)Nc2nncs2)C2(Cc3ccccc32)N=C(Nc2nc3ccccc3o2)N1] | in- active | in- active | >30 | 190.9 | 50.9 |
| | 2′-(benzo[d]oxazol-2-ylamino)-6′-methyl-N- | | | | | |
| | (1,3,4-thiadiazol-2-yl)-1′H- | | | | | |
| | spiro[bicyclo[4.2.0]octane-7,4′-pyrimidine]- | | | | | |
| | 1,3,5-triene-5′-carboxamide | | | | | |
| 260 | 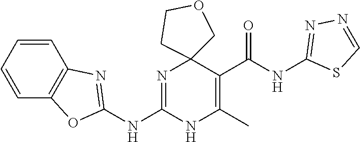 [SMILES: CC1=C(C(=O)Nc2nncs2)C2(CCOC2)N=C(Nc2nc3ccccc3o2)N1] | 12.19 (−1.2) | in- active | >30 | 102.7 | 37.9 |
| | 7-(benzo[d]oxazol-2-ylamino)-9-methyl-N- | | | | | |
| | (1,3,4-thiadiazol-2-yl)-2-oxa-6,8- | | | | | |
| | diazaspiro[4.5]deca-6,9-diene-10-carboxamide | | | | | |
| 261 | 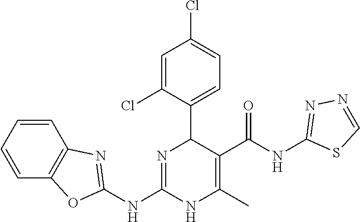 [SMILES: CC1=C(C(=O)Nc2nncs2)C(c2ccc(Cl)cc2Cl)N=C(Nc2nc3ccccc3o2)N1] | 4.85 | 6.11 | >30 | <4.9 | <1 |
| | 2-(benzo[d]oxazol-2-ylamino)-4-(2,4- | | | | | |
| | dichlorophenyl)-6-methyl-N-(1,3,4-thiadiazol-2- | | | | | |
| | yl)-1,4-dihydropyrimidine-5-carboxamide | | | | | |
| 262 | 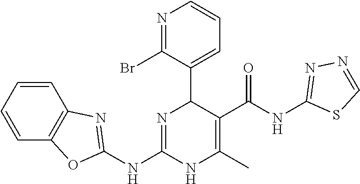 [SMILES: CC1=C(C(=O)Nc2nncs2)C(c2cccnc2Br)N=C(Nc2nc3ccccc3o2)N1] | 13.67 | 19.31 | >30 | 185.7 | 11.1 |
| | 2-(benzo[d]oxazol-2-ylamino)-4-(2- | | | | | |
| | bromopyridin-3-yl)-6-methyl-N-(1,3,4- | | | | | |
| | thiadiazol-2-yl)-1,4-dihydropyrimidine-5- | | | | | |
| | carboxamide | | | | | |
| 263 | 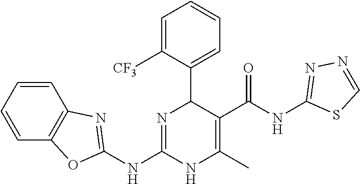 [SMILES: CC1=C(C(=O)Nc2nncs2)C(c2ccccc2C(F)(F)F)N=C(Nc2nc3ccccc3o2)N1] | 6.85 | 12.19 (−1.2) | >30 | 29.8 | <1 |
| | 2-(benzo[d]oxazol-2-ylamino)-6-methyl-N- | | | | | |
| | (1,3,4-thiadiazol-2-yl)-4-(2- | | | | | |
| | (trifluoromethyl)phenyl)-1,4-dihydropyrimidine- | | | | | |
| | 5-carboxamide | | | | | |
| 264 | 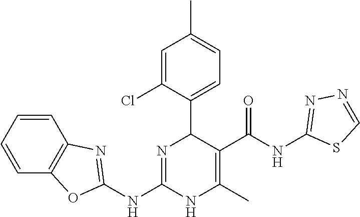 [SMILES: CC1=C(C(=O)Nc2nncs2)C(c2ccc(C)cc2Cl)N=C(Nc2nc3ccccc3o2)N1] | 3.06 | 6.11 | >30 | <14.4 | <1 |
| | 2-(benzo[d]oxazol-2-ylamino)-4-(2-chloro-4- | | | | | |
| | methylphenyl)-6-methyl-N-(1,3,4-thiadiazol-2- | | | | | |
| | yl)-1,4-dihydropyrimidine-5-carboxamide | | | | | |
| 265 | 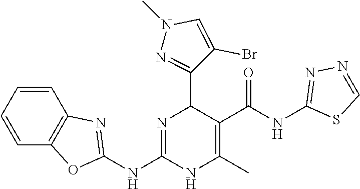 [SMILES: CC1=C(C(=O)Nc2nncs2)C(c2nn(C)cc2Br)N=C(Nc2nc3ccccc3o2)N1] | 3.43 | 19.31 | >30 | 1044.0 | 6.4 |
| | 2-(benzo[d]oxazol-2-ylamino)-4-(4-bromo-1- | | | | | |
| | methyl-1H-pyrazol-3-yl)-6-methyl-N-(1,3,4- | | | | | |
| | thiadiazol-2-yl)-1,4-dihydropyrimidine-5- | | | | | |
| | carboxamide | | | | | |
| 266 | 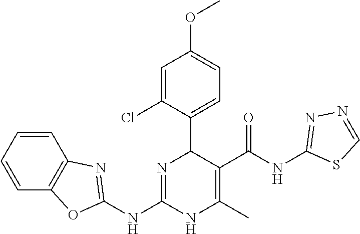 [SMILES: COc1ccc(C2N=C(Nc3nc4ccccc4o3)NC(C)=C2C(=O)Nc2nncs2)c(Cl)c1] | 3.85 | 5.44 (−1.2) | >30 | 31.9 | <1 |
| | 2-(benzo[d]oxazol-2-ylamino)-4-(2-chloro-4- | | | | | |
| | methoxyphenyl)-6-methyl-N-(1,3,4-thiadiazol-2- | | | | | |
| | yl)-1,4-dihydropyrimidine-5-carboxamide | | | | | |
| 267 | 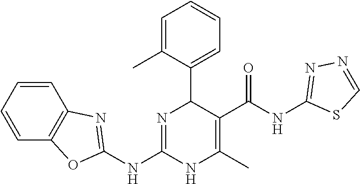 [SMILES: CC1=C(C(=O)Nc2nncs2)C(c2ccccc2C)N=C(Nc2nc3ccccc3o2)N1] | 12.19 (−1.2) | 21.67 (−1.2) | >30 | 83.8 | <1 |
| | 2-(benzo[d]oxazol-2-ylamino)-6-methyl-N- | | | | | |
| | (1,3,4-thiadiazol-2-yl)-4-(o-tolyl)-1,4- | | | | | |
| | dihydropyrimidine-5-carboxamide | | | | | |
| 268 | 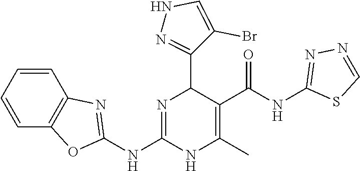 [SMILES: CC1=C(C(=O)Nc2nncs2)C(c2n[nH]cc2Br)N=C(Nc2nc3ccccc3o2)N1] | 7.69 | 19.31 (−1.2) | >30 | 27.4 | 11.4 |
| | 2-(benzo[d]oxazol-2-ylamino)-4-(4-bromo-1H- | | | | | |
| | pyrazol-3-yl)-6-methyl-N-(1,3,4-thiadiazol-2-yl)- | | | | | |
| | 1,4-dihydropyrimidine-5-carboxamide | | | | | |
| 269 | 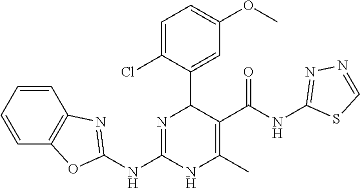 [SMILES: COc1ccc(Cl)c(C2N=C(Nc3nc4ccccc4o3)NC(C)=C2C(=O)Nc2nncs2)c1] | 13.67 | 17.21 (−1.2) | >30 | 19.8 | <1 |
| | 2-(benzo[d]oxazol-2-ylamino)-4-(2-chloro-5- | | | | | |
| | methoxyphenyl)-6-methyl-N-(1,3,4-thiadiazol-2- | | | | | |
| | yl)-1,4-dihydropyrimidine-5-carboxamide | | | | | |
| 270 | 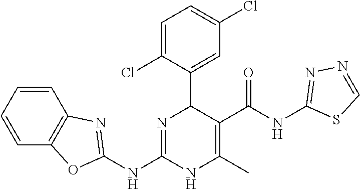 [SMILES: CC1=C(C(=O)Nc2nncs2)C(c2cc(Cl)ccc2Cl)N=C(Nc2nc3ccccc3o2)N1] | 6.11 (−1.2) | 10.86 (−1.2) | >30 | 2.9 | <1 |
| | 2-(benzo[d]oxazol-2-ylamino)-4-(2,5- | | | | | |
| | dichlorophenyl)-6-methyl-N-(1,3,4-thiadiazol-2- | | | | | |
| | yl)-1,4-dihydropyrimidine-5-carboxamide | | | | | |
| 271 | 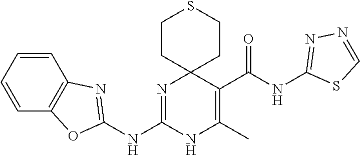 [SMILES: CC1=C(C(=O)Nc2nncs2)C2(CCSCC2)N=C(Nc2nc3ccccc3o2)N1] | 8.63 | 24.32 (−1.2) | >30 | 417.5 | 3.5 |
| | 2-(benzo[d]oxazol-2-ylamino)-4-methyl-N- | | | | | |
| | (1,3,4-thiadiazol-2-yl)-9-thia-1,3- | | | | | |
| | diazaspiro[5.5]undeca-1,4-diene-5- | | | | | |
| | carboxamide | | | | | |
| 272 | 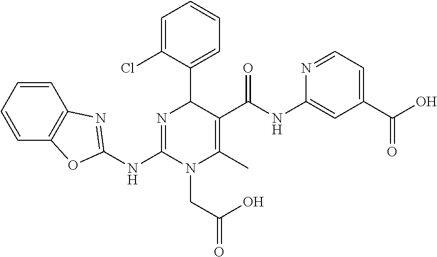 [SMILES: CC1=C(C(=O)Nc2cc(C(=O)O)ccn2)C(c2ccccc2Cl)N=C(Nc2nc3ccccc3o2)N1CC(=O)O] | 3.43 | 13.67 | >30 | na | >83 |
| | 2-(benzo[d]oxazol-2-ylamino)-4-methyl-N- | | | | | |
| | (1,3,4-thiadiazol-2-yl)-9-thia-1,3- | | | | | |
| | diazaspiro[5.5]undeca-1,4-diene-5- | | | | | |
| | carboxamide | | | | | |
| 273 | 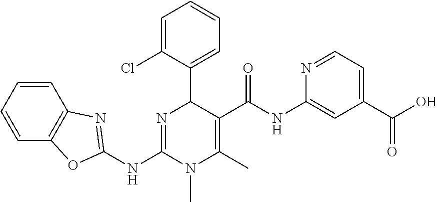 [SMILES: CC1=C(C(=O)Nc2cc(C(=O)O)ccn2)C(c2ccccc2Cl)N=C(Nc2nc3ccccc3o2)N1C] | 21.67 | 34.35 | >30 | na | >76 |
| | 2-(2-(benzo[d]oxazol-2-ylamino)-4-(2- | | | | | |
| | chlorophenyl)-1,6-dimethyl-1,4- | | | | | |
| | dihydropyrimidine-5-carboxamido)isonicotinic | | | | | |
| | acid | | | | | |
| 274 | 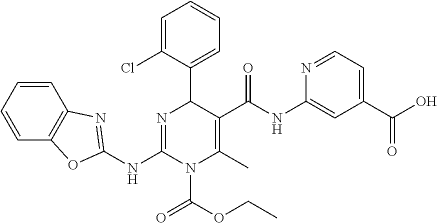 [SMILES: CCOC(=O)N1C(Nc2nc3ccccc3o2)=NC(c2ccccc2Cl)C(C(=O)Nc2cc(C(=O)O)ccn2)=C1C] | 9.68 | 19.31 (−1.2) | >30 | <1 | >85 |
| | 2-(2-(benzo[d]oxazol-2-ylamino)-4-(2- | | | | | |
| | chlorophenyl)-1-(ethoxycarbonyl)-6-methyl-1,4- | | | | | |
| | dihydropyrimidine-5-carboxamido)isonicotinic | | | | | |
| | acid | | | | | |
| 275 | 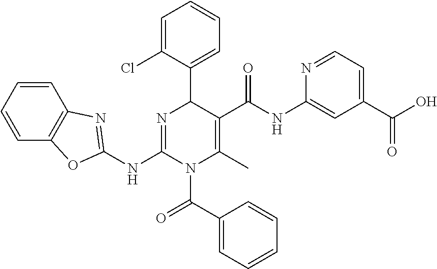 [SMILES: CC1=C(C(=O)Nc2cc(C(=O)O)ccn2)C(c2ccccc2Cl)N=C(Nc2nc3ccccc3o2)N1C(=O)c1ccccc1] | 3.43 | 7.69 | >30 | <1 | 73.9 |
| | 2-(2-(benzo[d]oxazol-2-ylamino)-1-benzoyl-4- | | | | | |
| | (2-chlorophenyl)-6-methyl-1,4- | | | | | |
| | dihydropyrimidine-5-carboxamido)isonicotinic | | | | | |
| | acid | | | | | |
| 276 | 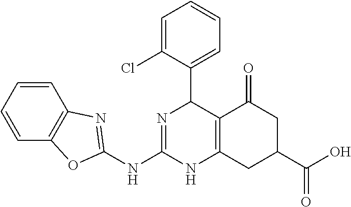 [SMILES: O=C1CC(C(=O)O)CC2=C1C(c1ccccc1Cl)N=C(Nc1nc3ccccc3o1)N2] | 3.85 | 6.85 | >30 | <1 | 52.63 |
| | 2-(benzo[d]oxazol-2-ylamino)-4-(2- | | | | | |
| | chlorophenyl)-5-oxo-1,4,5,6,7,8- | | | | | |
| | hexahydroquinazoline-7-carboxylic acid | | | | | |
| 277 | 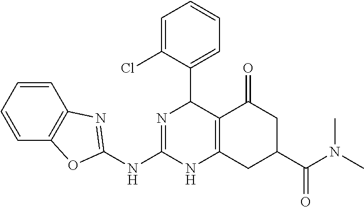 [SMILES: CN(C)C(=O)C1CC(=O)C2=C(C1)NC(Nc1nc3ccccc3o1)=NC2c1ccccc1Cl] | 3.43 | 4.32 | 20.79 | 219.68 | 10.84 |
| | 2-(benzo[d]oxazol-2-ylamino)-4-(2- | | | | | |
| | chlorophenyl)-N,N-dimethyl-5-oxo-1,4,5,6,7,8- | | | | | |
| | hexahydroquinazoline-7-carboxamide | | | | | |
| 278 | 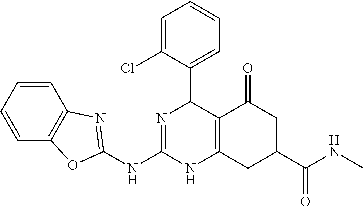 [SMILES: CNC(=O)C1CC(=O)C2=C(C1)NC(Nc1nc3ccccc3o1)=NC2c1ccccc1Cl] | 6.11 | 6.85 | >30 | 430.36 | <1 |
| | 2-(benzo[d]oxazol-2-ylamino)-4-(2- | | | | | |
| | chlorophenyl)-N-methyl-5-oxo-1,4,5,6,7,8- | | | | | |
| | hexahydroquinazoline-7-carboxamide | | | | | |
| 279 | 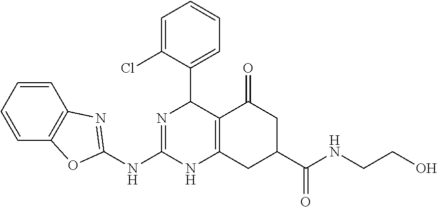 [SMILES: O=C1CC(C(=O)NCCO)CC2=C1C(c1ccccc1Cl)N=C(Nc1nc3ccccc3o1)N2] | 4.85 | 5.44 | >30 | <2 | 28.98 |
| | 2-(benzo[d]oxazol-2-ylamino)-4-(2- | | | | | |
| | chlorophenyl)-N-(2-hydroxyethyl)-5-oxo- | | | | | |
| | 1,4,5,6,7,8-hexahydroquinazoline-7- | | | | | |
| | carboxamide | | | | | |
| 280 | 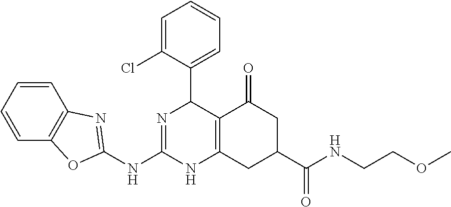 [SMILES: COCCNC(=O)C1CC(=O)C2=C(C1)NC(Nc1nc3ccccc3o1)=NC2c1ccccc1Cl] | 4.85 | 4.85 | 10.16 | 747.27 | 4.23 |
| | 2-(benzo[d]oxazol-2-ylamino)-4-(2- | | | | | |
| | chlorophenyl)-N-(2-methoxyethyl)-5-oxo- | | | | | |
| | 1,4,5,6,7,8-hexahydroquinazoline-7- | | | | | |
| | carboxamide | | | | | |
| 281 | 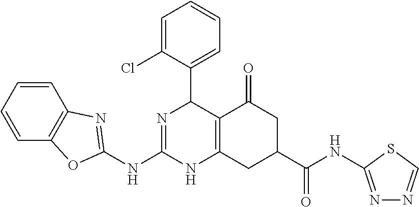 [SMILES: O=C1CC(C(=O)Nc2nncs2)CC2=C1C(c1ccccc1Cl)N=C(Nc1nc3ccccc3o1)N2] | 6.85 | 12.19 | >30 | <5 | 1.23 |
| | 2-(benzo[d]oxazol-2-ylamino)-4-(2- | | | | | |
| | chlorophenyl)-5-oxo-N-(1,3,4-thiadiazol-2-yl)- | | | | | |
| | 1,4,5,6,7,8-hexahydroquinazoline-7- | | | | | |
| | carboxamide | | | | | |
| 282 | 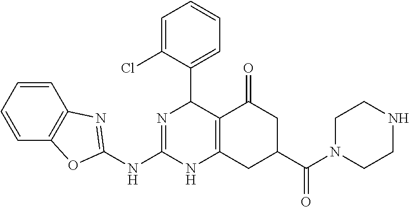 [SMILES: O=C1CC(C(=O)N2CCNCC2)CC2=C1C(c1ccccc1Cl)N=C(Nc1nc3ccccc3o1)N2] | 6.85 | 7.69 | 30 | <4.7 | 23.88 |
| | 2-(benzo[d]oxazol-2-ylamino)-4-(2- | | | | | |
| | chlorophenyl)-7-(piperazine-1-carbonyl)- | | | | | |
| | 4,5,6,8-tetrahydroquinazolin-5(1H)-one | | | | | |
| 283 | 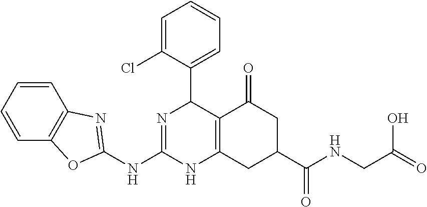 [SMILES: O=C(O)CNC(=O)C1CC(=O)C2=C(C1)NC(Nc1nc3ccccc3o1)=NC2c1ccccc1Cl] | 3.43 | 4.85 | >30 | <1 | 58.23 |
| | (2-(benzo[d]oxazol-2-ylamino)-4-(2- | | | | | |
| | chlorophenyl)-5-oxo-1,4,5,6,7,8- | | | | | |
| | hexahydroquinazoline-7-carbonyl)glycine | | | | | |
| 284 | 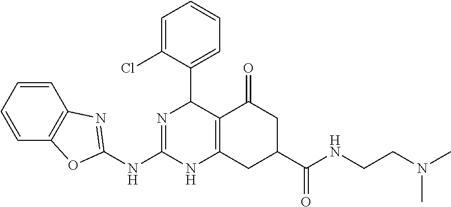 [SMILES: CN(C)CCNC(=O)C1CC(=O)C2=C(C1)NC(Nc1nc3ccccc3o1)=NC2c1ccccc1Cl] | 12.19 (−1.2) | 12.19 (−1.2) | >30 | <9.8 | 61.78 |
| | 2-(benzo[d]oxazol-2-ylamino)-4-(2- | | | | | |
| | chlorophenyl)-N-(2-(dimethylamino)ethyl)-5- | | | | | |
| | oxo-1,4,5,6,7,8-hexahydroquinazoline-7- | | | | | |
| | carboxamide | | | | | |

TABLE 2
Amine Modification

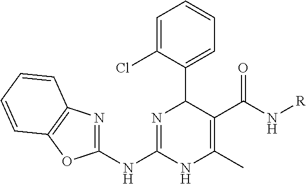

| Com- | | hGALK | mGALK |
|---|---|---|---|
| pound | R | IC₅₀ (μM) | IC₅₀ (μM) |
| 205 | 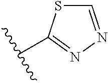 [SMILES: *c1nncs1] | 3.85 | 7.69 |
| 173 | 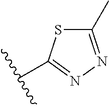 [SMILES: *c1nnc(C)s1] | 5.44 | 10.86 (−1.2) |
| 180 | 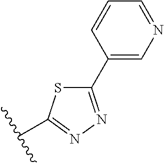 [SMILES: *c1nnc(-c2cccnc2)s1] | 3.06 | 9.68 |
| 187 | 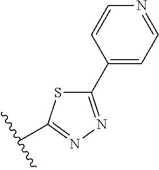 [SMILES: *c1nnc(-c2ccncc2)s1] | 10.86 (−1.2) | 19.31 (−1.2) |
| 168 | 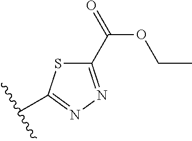 [SMILES: *c1nnc(C(=O)OCC)s1] | 4.85 | 10.86 |
| 174 | 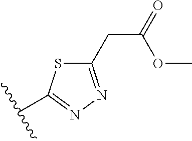 [SMILES: *c1nnc(CC(=O)OC)s1] | 6.11 | na |
| 185 | 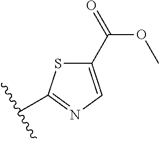 [SMILES: *c1ncc(C(=O)OC)s1] | 4.32 (−1.2) | 10.86 (−1.2) |
| 186 | 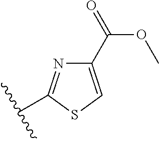 [SMILES: *c1nc(C(=O)OC)cs1] | 12.19 (−1.2) | 13.67 (−1.2) |
| 199 | 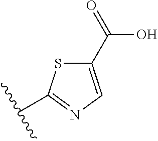 [SMILES: *c1ncc(C(=O)O)s1] | 1.72 | na |
| 200 | 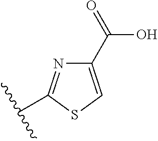 [SMILES: *c1nc(C(=O)O)cs1] | 9.68 | 19.31 (−1.2) |
| 193 | 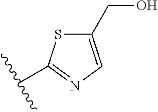 [SMILES: *c1ncc(CO)s1] | 12.19 (−1.2) | 19.31 (−1.2) |
| 198 | 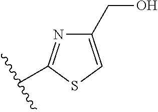 [SMILES: *c1nc(CO)cs1] | 17.21 | 19.31 (−1.2) |
| 167 | 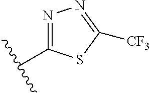 [SMILES: *c1nnc(C(F)(F)F)s1] | 12.19 | 21.67 |
| 232 | 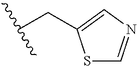 [SMILES: *Cc1cncs1] | 1.93 | 12.19 (−1.2) |
| 230 | 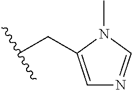 [SMILES: *Cc1cncn1C] | 1.72 | 6.85 (−1.2) |
| 231 | 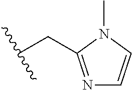 [SMILES: *Cc1nccn1C] | 15.34 | 13.67 |
| 233 | 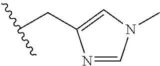 [SMILES: *Cc1cn(C)cn1] | 1.09 | 7.69 |
| 238 | 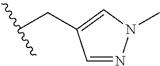 [SMILES: *Cc1cnn(C)c1] | 1.37 | 12.19 |

TABLE 3
Amine Modification

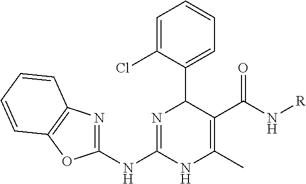

| Compound | R | hGALK IC₅₀ (μM) | mGALK IC₅₀ (μM) |
|---|---|---|---|
| 137 | Ph | 10.86 (−1.2) | 13.67 (−1.2) |
| 157 | 2-F-Ph | 7.69 (−1.2) | 9.68 (−1.2) |
| 158 | 3-F-Ph | 6.85 (−1.2) | 12.19 (−1.2) |
| 148 | 4-F-Ph | 7.69 (−1.2) | 12.19 (−1.2) |
| 140 | 2-Cl-Ph | 4.32 (−1.2) | inactive |
| 154 | 3-Cl-Ph | inactive | inactive |
| 141 | 4-Cl-Ph | inactive | 7.69 (−1.2) |
| 147 | 3-Br-Ph | inactive | inactive |
| 150 | 4-Br-Ph | 9.68 (−1.4) | 15.34 (−1.2) |
| 161 | 2-OH-Ph | 6.85 | 17.21 (−1.2) |
| 162 | 4-OH-Ph | 3.85 | 9.68 |
| 156 | 2-CH2OH-Ph | 3.85 | 17.21 (−1.2) |
| 144 | 3-CH2OH-Ph | 3.85 | 9.68 |
| 160 | 4-CH2OH-Ph | 17.21 (−2.2) | na |
| 159 | 2-CO2H-Ph | 34.35 (−3) | na |
| 142 | 3-CO2H-Ph | 2.43 | 6.11 |
| 143 | 4-CO2H-Ph | 1.53 | 6.11 |
| 146 | 2-CO2Me-Ph | inactive | inactive |
| 139 | 3-CO2Et-Ph | 3.85 (−1.2) | na |
| 138 | 4-CO2Me-Ph | 4.32 (−1.2) | 9.68 (−1.2) |
| 214 | 2-py | 4.32 (−1.2) | 10.86 (−1.2) |
| 149 | 3-py | 6.85 | 12.19 |

TABLE 4
Amine Modification

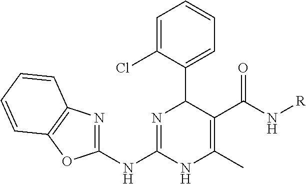

| Compound | R | hGALK IC₅₀ (μM) | mGALK IC₅₀ (μM) |
|---|---|---|---|
| 217 | 4-F-Bn | 7.69 (−1.2) | inactive |
| 216 | 2-Cl-Bn | inactive | inactive |
| 218 | 4-Cl-Bn | inactive | inactive |
| 215 | 2-OMe-Bn | 7.69 (−1.2) | 12.19 (−1.2) |
| 219 | 2-OH-Bn | 6.85 (−1.2) | 10.86 (−1.2) |
| 229 | 2-CH2OH-Bn | 6.11 | 15.34 (−1.2) |
| 227 | 3-CH2OH-Bn | 4.85 | 12.19 |
| 228 | 4-CH2OH-Bn | 6.85 | 15.34 (−1.2) |
| 226 | 3-CO2H-Bn | 7.69 | 19.31 (−1.2) |
| 225 | 4-CO2H-Bn | 3.43 | 10.86 (−1.2) |
| 222 | 2-CO2Me-Bn | 6.85 (−1.2) | 4.32 (−1.2) |
| 224 | 3-CO2Me-Bn | 4.85 (−1.4) | 7.69 (−1.2) |
| 223 | 4-CO2Me-Bn | 4.32 (−1.2) | 8.63 (−1.2) |
| 220 | 2-Bnpy | 8.63 | 19.31 (−1.2) |
| 221 | 3-Bnpy | 6.85 | 19.31 (−1.2) |

TABLE 5
Amine Modification

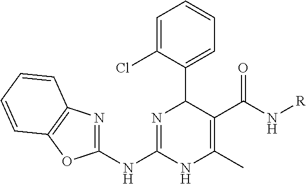

| Compound | R | hGALK IC50 (μM) | mGALK IC50 (μM) |
|---|---|---|---|
| 237 | 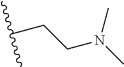 [SMILES: *CCN(C)C] | 19.31 (−1.2) | 15.34 (−1.2) |
| 152 | 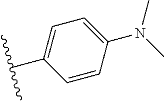 [SMILES: *c1ccc(N(C)C)cc1] | 7.69 (−1.2) | 13.67 (−1.2) |
| 190 | 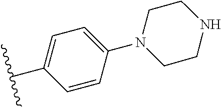 [SMILES: *c1ccc(N2CCNCC2)cc1] | 68.53 (−1.2) | 48.51 (−2.2) |
| 182 | 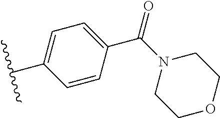 [SMILES: *c1ccc(C(=O)N2CCOCC2)cc1] | 19.31 | 21.67 |
| 183 | 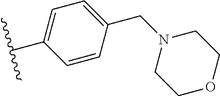 [SMILES: *c1ccc(CN2CCOCC2)cc1] | 5.44 | 9.68 (−1.2) |
| 172 | 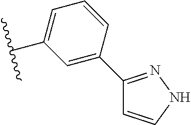 [SMILES: *c1cccc(-c2cc[nH]n2)c1] | 7.69 | inactive |
| 178 | 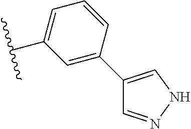 [SMILES: *c1cccc(-c2cn[nH]c2)c1] | 17.21 (−1.2) | 38.54 (−2.2) |
| 175 | 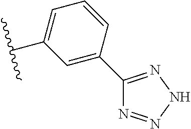 [SMILES: *c1cccc(-c2nn[nH]n2)c1] | 19.31 | 34.35 (−2.2) |
| 184 | 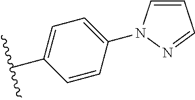 [SMILES: *c1ccc(-n2cccn2)cc1] | 6.11 (−1.2) | 12.19 (−1.2) |
| 191 | 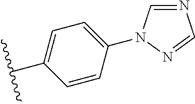 [SMILES: *c1ccc(-n2cncn2)cc1] | 24.32 (−1.2) | 34.35 (−1.2) |
| 192 | 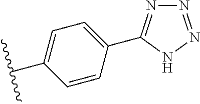 [SMILES: *c1ccc(-c2nnn[nH]2)cc1] | 4.85 | na |
| 179 | 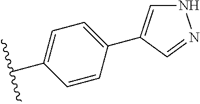 [SMILES: *c1ccc(-c2cn[nH]c2)cc1] | 6.85 (−1.2) | 12.19 (−1.2) |
| 176 | 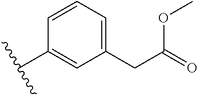 [SMILES: *c1cccc(CC(=O)OC)c1] | 10.86 (−1.2) | 13.67 (−1.2) |
| 171 | 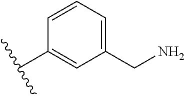 [SMILES: *c1cccc(CN)c1] | 10.86 | 6.85 |
| 177 | 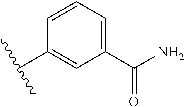 [SMILES: *c1cccc(C(N)=O)c1] | 7.69 | 17.21 (−1.2) |
| 201 | 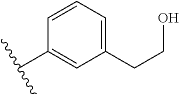 [SMILES: *c1cccc(CCO)c1] | 17.21 (−2.2) | na |
| 170 | 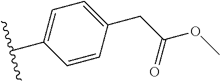 [SMILES: *c1ccc(CC(=O)OC)cc1] | 5.44 | 12.19 (−1.2) |
| 169 | 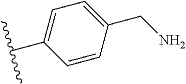 [SMILES: *c1ccc(CN)cc1] | 6.11 | 4.32 |
| 181 | 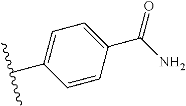 [SMILES: *c1ccc(C(N)=O)cc1] | 4.32 | 6.85 |
| 234 | 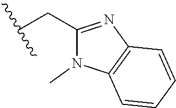 [SMILES: *Cc1nc2ccccc2n1C] | 8.63 (−1.2) | 13.67 (−1.2) |
| 155 | 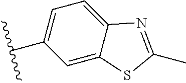 [SMILES: *c1ccc2nc(C)sc2c1] | 5.44 (−1.2) | 13.67 (−1.2) |
| 163 | 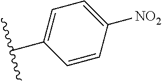 [SMILES: *c1ccc([N+](=O)[O-])cc1] | 3.43 (−1.2) | 3.43 (−1.2) |
| 189 | 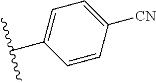 [SMILES: *c1ccc(C#N)cc1] | 10.86 (−1.2) | 13.67 (−1.2) |

TABLE 6
Amine Modification

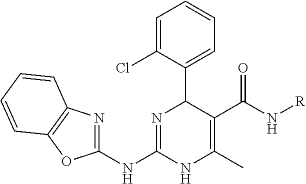

| | | hGALK | mGALK |
|---|---|---|---|
| Com- | | IC50 | IC50 |
| pound | R | (μM) | (μM) |
| 204 | 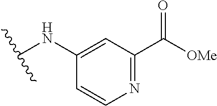 [SMILES: *Nc1ccnc(C(=O)OC)c1] | 7.69 | 10.86 |
| 203 | 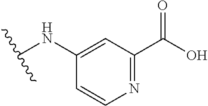 [SMILES: *Nc1ccnc(C(=O)O)c1] | 3.43 | 3.06 |
| 188 | 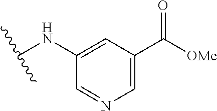 [SMILES: *Nc1cncc(C(=O)OC)c1] | 21 (−2.1) | na |
| 202 | 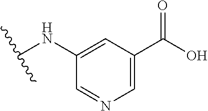 [SMILES: *Nc1cncc(C(=O)O)c1] | 4.32 | na |
| 197 | 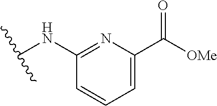 [SMILES: *Nc1cccc(C(=O)OC)n1] | 9.68 (−1.2) | 12.19 (−1.2) |
| 195 | 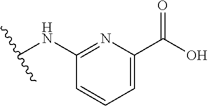 [SMILES: *Nc1cccc(C(=O)O)n1] | 4.32 | 6.85 |
| 196 | 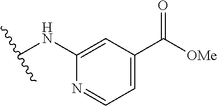 [SMILES: *Nc1cc(C(=O)OC)ccn1] | 2.73 | 5.44 (−1.2) |
| 194 | 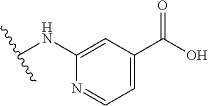 [SMILES: *Nc1cc(C(=O)O)ccn1] | 1.08 | 6.85 |
| 211 | 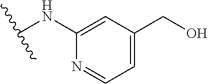 [SMILES: *Nc1cc(CO)ccn1] | 3.06 | 9.68 |
| 212 | 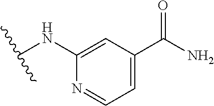 [SMILES: *Nc1cc(C(N)=O)ccn1] | 2.43 | 7.69 |
| 213 | 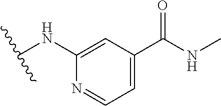 [SMILES: *Nc1cc(C(=O)NC)ccn1] | 3.06 | 10.86 |

TABLE 7
Amine Modification

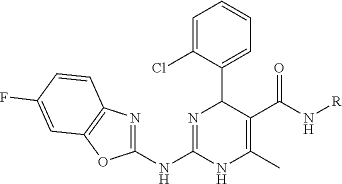

| | | hGALK | mGALK |
|---|---|---|---|
| Com- | | IC50 | IC50 |
| pound | R | (μM) | (μM) |
| 1 | 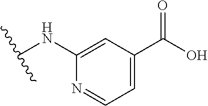 [SMILES: *Nc1cc(C(=O)O)ccn1] | 0.68 | 4.32 |
| 37 | 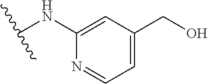 [SMILES: *Nc1cc(CO)ccn1] | 3.06 | 10.86 |
| 38 | 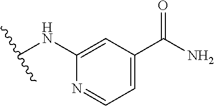 [SMILES: *Nc1cc(C(N)=O)ccn1] | 4.85 | 19.31 (−2.1) |
| 39 | 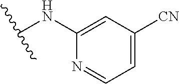 [SMILES: *Nc1cc(C#N)ccn1] | 4.32 (−2.1) | 4.85 (−2.1) |
| 41 | 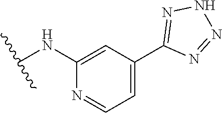 [SMILES: *Nc1cc(-c2nn[nH]n2)ccn1] | 0.77 | 7.69 |

TABLE 8
Benzoxazole Modification

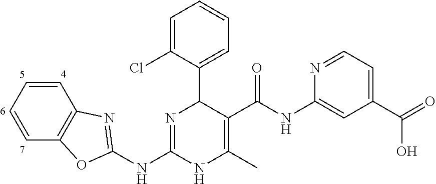 [SMILES: CC1=C(C(=O)Nc2cc(C(=O)O)ccn2)C(c2ccccc2Cl)N=C(Nc2nc3ccccc3o2)N1]

| | | hGALK | mGALK |
|---|---|---|---|
| Compound | R | IC50 (μM) | IC50 (μM) |
| 194 | Benzoxazole | 1.08 | 6.85 |
| 8 | 4-F Benzoxazole | 3.85 | 21.67 (−1.2) |
| 13 | 5-F Benzoxazole | 3.43 | 17.21 |
| 1 | 6-F Benzoxazole | 0.68 | 4.32 |
| 10 | 7-F Benzoxazole | 1.72 | 8.63 |
| 11 | 4-Cl Benzoxazole | 5.44 | 13.67 (−1.2) |
| 9 | 5-Cl Benzoxazole | 2.43 | 6.11 |
| 14 | 6-Cl Benzoxazole | 1.93 | 6.11 |
| 17 | 7-Cl Benzoxazole | 1.93 | 9.68 |
| 18 | 4-Me Benzoxazole | 9.68 | 17.21 (−1.2) |
| 4 | 5-Me Benzoxazole | 1.37 | 3.06 |
| 6 | 6-Me Benzoxazole | 5.44 | 17.21 (−1.2) |
| 15 | 7-Me Benzoxazole | 0.97 | 6.85 |
| 5 | 5-OMe Benzoxazole | 6.85 | 12.19 (−1.2) |
| 7 | 6-OMe Benzoxazole | 21.67 (−1.2) | 21.67 (−1.2) |
| 22 | 7-OMe Benzoxazole | 21.67 (−1.2) | inactive |
| 2 | 4-NH2 Benzoxazole | 21.67 (−1.2) | 30.61 (−1.2) |
| 12 | 5-NH2 Benzoxazole | 7.69 | 21.67 (−1.2) |
| 3 | 7-NH2 Benzoxazole | 0.22 | 3.43 |
| 16 | 7-CF3 Benzoxazole | 21.67 (−1.2) | 48.52 (−2.2) |
| 21 | 7-OCF3 Benzoxazole | inactive | inactive |
| 23 | 7-NO2 Benzoxazole | 19.31 (−1.2) | 38.54 (−1.4) |

TABLE 9
Aryl Modification

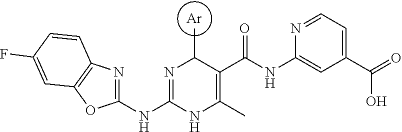

| | | hGALK | mGALK |
|---|---|---|---|
| Compound | R | IC50 (μM) | IC50 (μM) |
| 1 | 2-Cl—Ph | 0.68 | 4.32 |
| 75 | 2-Br—Ph | 0.54 | 3.85 |
| 77 | 3-CF3—Ph | 19.31 | inactive |
| | | (−1.2) | |
| 76 | 3-CO2NH2—Ph | 7.69 | 21.67 |
| | | | (−1.2) |
| 78 | 3-Sulfonamide-Ph | 17.21 | 38.54 |
| | | (−1.2) | (−2.2) |
| 48 | 2-Cl, 3-F—Ph | 4.85 | 15.34 |
| | | | (−1.2) |
| 49 | 2-Cl, 3-Cl—Ph | 8.63 | 21.67 |
| | | | (−1.2) |
| 52 | 2-Cl, 3-OMe—Ph | 6.85 | 19.31 |
| | | | (−1.2) |
| 51 | 2-Cl, 3-CF3—Ph | 19.31 | 27.28 |
| | | (−1.2) | (−1.2) |
| 64 | 2-Cl, 3-NH2—Ph | 3.06 | 17.21 |
| 45 | 2-Cl, 4-F—Ph | 0.86 | 4.32 |
| 47 | 2-Cl, 4-Cl—Ph | 1.09 | 3.85 |
| 50 | 2-Cl, 4-Me—Ph | 0.39 | 3.06 |
| 46 | 2-Cl, 4-OMe—Ph | 0.43 | 3.06 |
| 65 | 2-Cl, 4—Ph—Ph | 3.43 | na |
| 62 | 2-Cl, 4-Br—Ph | 1.09 | 3.85 |
| 66 | 2-Cl, 4-CN—Ph | 0.61 | 3.85 |
| 72 | 2-Cl, 4-CO2H—Ph | 2.17 | 15.34 |
| | | | (−1.2) |
| 61 | 2-Cl, 4-CF3—Ph | 1.53 | 10.86 |
| 67 | 2-Cl, 4-OEt—Ph | 0.69 | 3.85 |
| 60 | 2-Cl, 4-imidazol-Ph | 0.77 | 3.43 |
| 59 | 2-Cl, 4-morpholino-Ph | 0.43 | 3.85 |
| 74 | 2-Cl, 4-(1H- | 0.12 | 0.97 |
| | pyrazol-1-yl)—Ph | | |
| 58 | 2-Cl, 4-piperidin-Ph | 1.22 | 12.19 |
| 57 | 2-Cl, pyrrolidin-Ph | 1.09 | 10.86 |
| 68 | 2-Cl, 4-(trifluoromethyl)- | 7.69 | 21.67 |
| | [1,1′-biphenyl]-4-yl)—Ph | | |
| 69 | 2-Cl, 4-(6- | 0.34 | 1.22 |
| | methylpyridine-3-yl)—Ph | | |
| 70 | 2-Cl, 4-(6-(trifluoromethyl) | 1.21 | 3.85 |
| | pyridin-3-yl)—Ph | | |
| 71 | 2-Cl, 4-(2- | 0.11 | 0.69 |
| | methylpyrimidin-5-yl)—Ph | | |
| 73 | 2-Cl, 5-CO2H—Ph | 1.72 | 12.19 |

TABLE 10
Benzoxazole Substitution

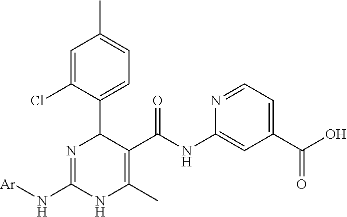

| | | hGALK | mGALK |
|---|---|---|---|
| Compound | R | IC50 (μM) | IC50 (μM) |
| 102 | 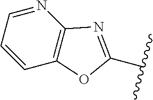 [SMILES: *c1nc2ncccc2o1] | 4.85 | 19.31 (−1.2) |
| 106 | 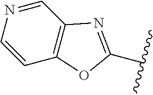 [SMILES: *c1nc2cnccc2o1] | 0.86 | 6.85 |
| 108 | 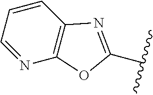 [SMILES: *c1nc2cccnc2o1] | 12.19 (−1.2) | inactive |
| 109 | 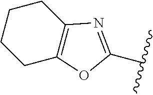 [SMILES: *c1nc2c(o1)CCCC2] | 3.06 | 4.32 (−1.2) |
| 103 | 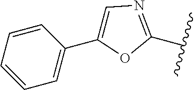 [SMILES: *c1ncc(-c2ccccc2)o1] | 24.32 (−1.2) | 43.24 (−1.2) |

TABLE 11
Benzoxazole Substitution

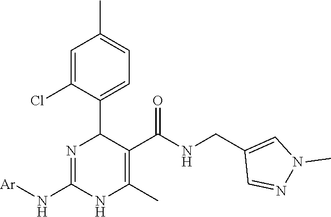

| | | hGALK | mGALK |
|---|---|---|---|
| Compound | R | IC50 (μM) | IC50 (μM) |
| 105 | 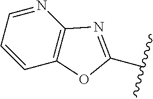 [SMILES: *c1nc2ncccc2o1] | 15.34 (−1.2) | inactive |
| 104 | 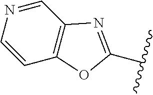 [SMILES: *c1nc2cnccc2o1] | 3.06 | 12.19 (1.2) |
| 107 | 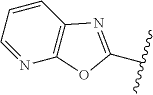 [SMILES: *c1nc2cccnc2o1] | 17.21 (−1.2) | inactive |

TABLE 12
Amine Modification

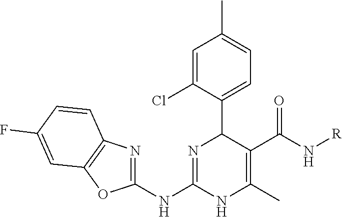

| | | hGALK | mGALK |
|---|---|---|---|
| Com- | | IC50 | IC50 |
| pound | R | (μM) | (μM) |
| 90 | 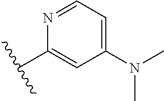 [SMILES: *c1cc(N(C)C)ccn1] | 4.32 (−1.2) | 6.85 (−1.2) |
| 111 | 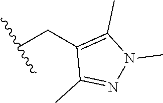 [SMILES: *Cc1c(C)nn(C)c1C] | 3.85 | 4.85 |
| 112 | 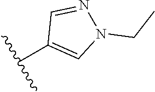 [SMILES: *c1cnn(CC)c1] | 1.22 | 6.85 |
| 124 | 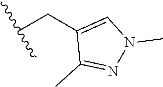 [SMILES: *Cc1cn(C)nc1C] | 0.49 | 3.43 |
| 110 | 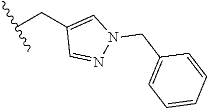 [SMILES: *Cc1cnn(Cc2ccccc2)c1] | 1.72 | 5.44 (−1.2) |
| 92 | 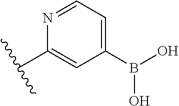 [SMILES: *c1cc(B(O)O)ccn1] | 1.93 | 10.86 |

TABLE 13
Amine Modification

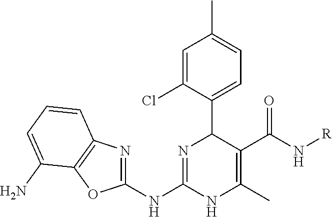

| | | hGALK | mGALK |
|---|---|---|---|
| Com- | | IC50 | IC50 |
| pound | R | (μM) | (μM) |
| 89 | 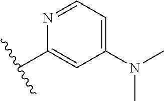 [SMILES: *c1cc(N(C)C)ccn1] | 3.43 | 15.34 (−1.2) |
| 113 | 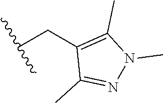 [SMILES: *Cc1c(C)nn(C)c1C] | 1.22 | 6.85 |
| 117 | 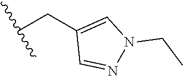 [SMILES: *Cc1cnn(CC)c1] | 0.17 | 3.06 |
| 114 | 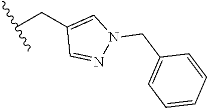 [SMILES: *Cc1cnn(Cc2ccccc2)c1] | 0.39 | 5.44 (−1.2) |
| 122 | 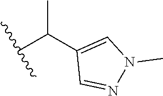 [SMILES: *C(C)c1cnn(C)c1] | 0.55 | 5.44 |

TABLE 14
Amine Modification

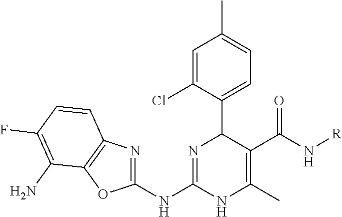

| | | hGALK | mGALK |
|---|---|---|---|
| Com- | | IC50 | IC50 |
| pound | R | (μM) | (μM) |
| 126 | 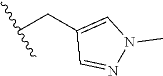 [SMILES: *Cc1cnn(C)c1] | 0.05 | 0.69 |
| 125 | 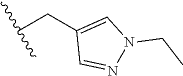 [SMILES: *Cc1cnn(CC)c1] | 0.05 | 0.61 |
| 129 | 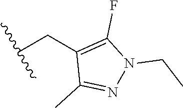 [SMILES: *Cc1c(C)nn(CC)c1F] | 0.43 | 2.17 |
| 131 | 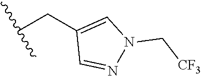 [SMILES: *Cc1cnn(CC(F)(F)F)c1] | 0.19 | 3.06 |
| 132 | 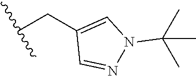 [SMILES: *Cc1cnn(C(C)(C)C)c1] | 0.77 | 4.32 |
| 133 | 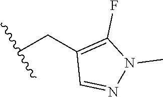 [SMILES: *Cc1cnn(C)c1F] | 0.11 | 1.09 |

TABLE 15
Enantiomer Activity

| | | hGALK | mGALK |
|---|---|---|---|
| Compound | R | IC50 (μM) | IC50 (μM) |
| 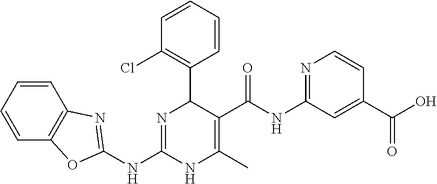 [SMILES: CC1=C(C(=O)Nc2cc(C(=O)O)ccn2)C(c2ccccc2Cl)N=C(Nc2nc3ccccc3o2)N1] | 194 (Racemate) 208 (R) 209 (S) | 1.08 0.54 12.19 | 6.85 3.43 19.31 (−1.2) |
| 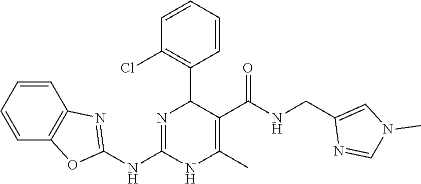 [SMILES: CC1=C(C(=O)NCc2cn(C)cn2)C(c2ccccc2Cl)N=C(Nc2nc3ccccc3o2)N1] | 233 (Racemate) 235 (R) 236 (S) | 1.09 3.06 inactive | 7.69 4.32 inactive |
| 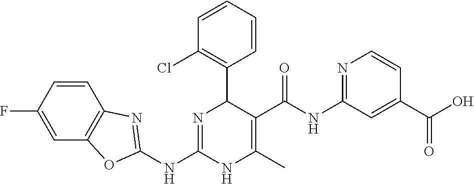 [SMILES: CC1=C(C(=O)Nc2cc(C(=O)O)ccn2)C(c2ccccc2Cl)N=C(Nc2nc3ccc(F)cc3o2)N1] | 1 (Racemate) 19 (R) 20 (S) | 068 0.24 3.06 | 4.32 1.72 13.67 (−1.2) |
| 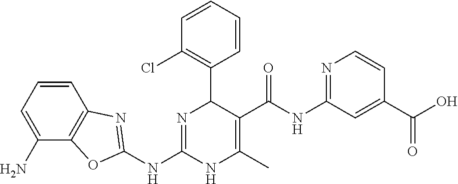 [SMILES: CC1=C(C(=O)Nc2cc(C(=O)O)ccn2)C(c2ccccc2Cl)N=C(Nc2nc3cccc(N)c3o2)N1] | 3 (Racemate) 24 (R) 25 (S) | 0.22 0.08 6.11 | 3.43 1.72 34.35 (−2.2) |
| 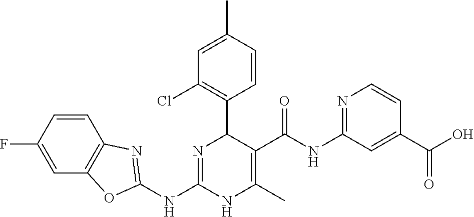 [SMILES: CC1=C(C(=O)Nc2cc(C(=O)O)ccn2)C(c2ccc(C)cc2Cl)N=C(Nc2nc3ccc(F)cc3o2)N1] | 50 (Racemate) 55 (R) 56 (S) | 0.39 015 1.93 | 3.06 1.37 6.85 |
| 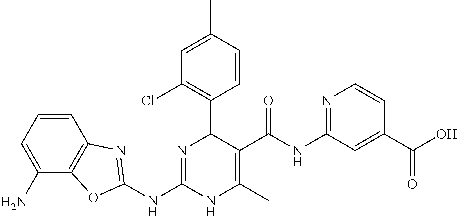 [SMILES: CC1=C(C(=O)Nc2cc(C(=O)O)ccn2)C(c2ccc(C)cc2Cl)N=C(Nc2nc3cccc(N)c3o2)N1] | 79 (Racemate) 85 (R) 84 (S) | 0.12 0.09 3.43 | 1.72 1.41 21.67 (−1.2) |
| 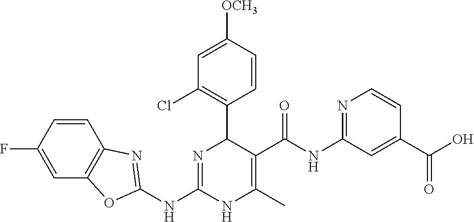 [SMILES: COc1ccc(C2N=C(Nc3nc4ccc(F)cc4o3)NC(C)=C2C(=O)Nc2cc(C(=O)O)ccn2)c(Cl)c1] | 46 (Racemate) 53 (R) 54 (S) | 0.43 0.25 4.84 | 3.06 1.99 19.31 (−1.2) |
| 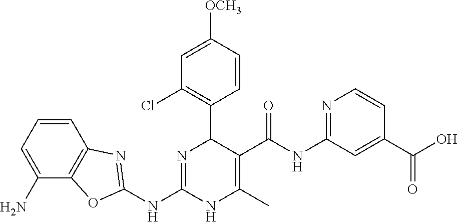 [SMILES: COc1ccc(C2N=C(Nc3nc4cccc(N)c4o3)NC(C)=C2C(=O)Nc2cc(C(=O)O)ccn2)c(Cl)c1] | 80 (Racemate) 87 (R) 86 (S) | 0.14 0.15 5.44 | 1.93 2.43 34.35 (−2.2) |
| 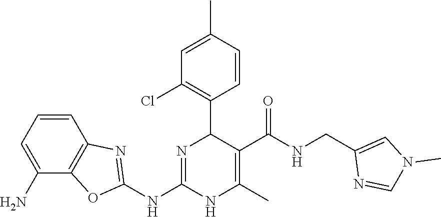 [SMILES: CC1=C(C(=O)NCc2cn(C)cn2)C(c2ccc(C)cc2Cl)N=C(Nc2nc3cccc(N)c3o2)N1] | 83 (Racemate) 115 (R) 116 (S) | 0.22 0.13 12.19 | 2.17 1.58 30.61 (−1.2) |
| 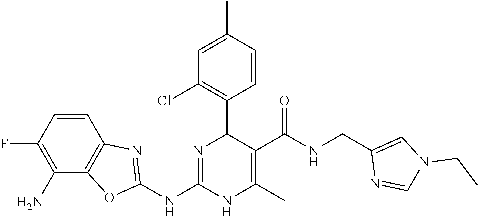 [SMILES: CCn1cnc(CNC(=O)C2=C(C)NC(Nc3nc4ccc(F)c(N)c4o3)=NC2c2ccc(C)cc2Cl)c1] | 125 (Racemate) 135 (R) 134 (S) | 0.05 0.07 1.53 | 0.61 0.97 10.86 (−1.2) |

### Example 12

Crystallographic Analysis of hGALK1m
Purification of hGALK1m

Human GALK1 (SEQ ID NO: 2) cDNA (SEQ ID NO: 1, nucleotides 57-1235) was cloned to the pET21d vector. Through the Surface Entropy Reduction prediction (SERp) server (service.mbi.ucla.edu/sSER) Lys252 and Glu253 were identified as potential high surface entropy residues, which were mutated to alanine through site-directed mutagenesis to produce hGALK1m (i.e., hGALK1 K252A, E253A; SEQ ID NO:3). Then the plasmid was transfected to E. coli HMS174. A single colony was selected and grown in 2 mL of LB media overnight at 37° C. The next day, 200 μL of the overnight culture was inoculated into 1 L of LB media and cultured in 37° C. at 150 RPM, until the OD₆₀₀ reached 0.9, before 1 mM of IPTG was added and the culture was shifted to 19° C. The bacteria were further cultured overnight before collection by centrifugation. The pellet was stored at −80° C. The pellet was thawed in ice cold protein lysis buffer containing 50 mM sodium phosphate buffer at pH 8, 100 mM galactose, 5% glycerol, 300 mM NaCl, and 20 mM imidazole. Cells were disrupted by combining lysozyme/DNase treatment and sonication. The lysate was cleared with centrifugation and the supernatant was incubated with nickel beads for 1 hour, which had been preequilibrated with the lysis buffer. Then the nickel beads were washed with lysis buffer and the protein was eluted with elution buffer, which containing 200 mM imidazole and the remaining components of the lysis buffer. The protein elution was loaded onto a SD200 size exclusion column to further purify hGALK1m. The column buffer contained 50 mM HEPES at pH 8, 100 mM galactose, 5% glycerol, and 200 mM NaCl. The fractions containing target protein were combined and concentrated to 18 mg/mL using an Amicon spin concentrator with 10 kDa molecular weight cutoff. The purity of the final protein solution was determined to be approximately 90%.

Co-Crystallization of hGALK1m and Compounds

Crystals were grown by vapor diffusion in sitting drop crystallization plates. The initial crystallization conditions were screened using Hampton Research Phosphate Buffer Screen with concentrations of the buffer ranging from 1.0 M Na/K to 3.2 M Na/K and pH from 5.8 to 7.6. Crystals normally grew in the buffer from 1.8 M Na/K to 2.6 M Na/K and pH from 6.0 to 6.8. When setting up the drops, the ratios of the protein solution and the crystallization solution were 1:2, 1:1 and 2:1, all of which permitted crystal growth in different crystallization solutions. The co-crystallization of the compounds and protein was done with a seeding technique. Crystals were obtained by co-crystallizing hGALK1m, galactose, and ADP (10 mM to 20 mM), which served as seeds for co-crystallizing hGALK1m and compounds. The seeding was performed one day after the drops were set. The molar ratio depended on the solubility of the compounds but was at least 1:2 and could be as great as to 1:4 to 1:10. The DMSO concentration was no higher than 2.5%. Normally, the crystals appeared within 7 days after the seeding and would grow over the 1 to 2 weeks. The crystals were harvested into a cryo-preservation solution that was 2.4 M Na/K at a pH of 6.4. The crystals were diffracted at the Stanford Synchrotron Radiation Lightsource (SSRL), and data were collected. Data were indexed, integrated, and scaled with the HKL2000 package, with subsequent density modification using RESOLVE. Model building into the experimental map was performed manually with COOT and the model was refined using REFMAC5 (CCP4). Crystallographic data and refinement statistics are shown in Table 16.

TABLE 16
Crystallographic Data and Refinement Statistics
hGALK1m complexes

| Ligand Name | ADP | Compound 194 | Compound A |
|---|---|---|---|
| Source/Wavelength | SSRL 7-1/1.15330 Å | SSRL 7-1/1.0000 Å | SSRL 7-1/1.2708 Å |
| Indexing/Scaling | XDS/AIMLESS | XDS/AIMLESS | XDS/AIMLESS |
| programs | | | |
| Space Group (unit | P6₁22 (128.77, | P6₁22 (128.24, | P6₁22 (129.22, |
| cell dimensions) | 128.77, 239.63) | 128.24, 239.96) | 129.22, 240.79) |
| Resolution (Å) | 40.0-2.10 | 40.0-2.13 | 40.0-2.20 |
| Resolution (Å) (high- | (2.15-2.10) | (2.18-2.13) | (2.25-2.20) |
| resolution shell) | | | |
| Reflections | 1,148,425 | 2,651,831 | 1,283,748 |
| measured | | | |
| Unique reflections | 68,728 | 65,674 | 61,072 |
| Completeness (%) | 99.3 (92.0) | 99.5 (93.5) | 99.8 (97.2) |
| (high-resolution shell) | | | |
| Redundancy (high- | 16.7 (5.3) | 40.4 (24.4) | 21.0 (13.5) |
| resolution shell) | | | |
| <//σ/> (high- | 9.4 (1.2) | 21.0 (2.3) | 21.3 (2.9) |
| resolution shell) | | | |
| <CC½> | 0.997 (0.568) | 0.999 (0.704) | 0.999 (0.842) |
| Rₚᵢₘ (high-resolution | 0.046 (0.515) | 0.053 (0.310) | 0.024 (0.247) |
| shell) | | | |
| Mosaicity (°) | 0.24 | 0.23 | 0.15 |

Refinement

| | | | |
|---|---|---|---|
| Program | Phenix.refine | Phenix.refine | Phenix.refine |
| Resolution (Å) | 40.00-2.10 | 40.00-2.13 | 40.00-2.20 |
| Resolution (Å)-(high- | 2.13-2.10 | 2.16-2.13 | 2.25-2.20 |
| resolution shell) | | | |
| No. Reflections | 68,594 | 65,535 | 60,970 |
| Reflections in R_{free} | 3072 | 3278 | 2268 |
| set | | | |
| R_{cryst} (high-resolution | 0.195 (0.346) | 0.196 (0.303) | 0.174 (0.223) |
| shell) | | | |
| R_{free} (high-resolution | 0.233 (0.375) | 0.242 (0.372) | 0.227 (0.270) |
| shell) | | | |
| RMSD: bonds (Å)/ | 0.008/1.029 | 0.014/1.511 | 0.013/1.399 |
| angles (°) | | | |
| <B> (Å²): all atoms/ | 55.84/6198 | 60.79/6427 | 48.71/6590 |
| # atoms | | | |
| <B> (Å²): water | 53.13/178 | 55.14/310 | 51.24/496 |
| molecules/#water | | | |
| <B> (Å²): Ligand | 43.60 | 60.02 | 44.17 |
| ϕ/ψ most favored (%)/ | | | |
| additionally allowed | 96.8/3.0 | 97.2/2.70 | 96.1/3.5 |
| (%) | | | |

hGALK1 In Vitro Biochemical and Cellular Inhibitory Activity Analyses

The in vitro hGALK1 activity assay was performed as previously described. Liu et al., Bioorg. Med. Chem. Lett. 25(3): 721-727 (2015). For the measurement of cellular Gal-1P reduction, compounds at different concentrations were added to cultured patient fibroblast at 80% confluency. After incubating the compounds for 16 hours, 10 mM galactose was added to the culture. After another 4 hours, cells were washed with cold PBS buffer 3× and harvested for gal-1P measurement, according to the previously published method. Liu et al., Bioorg. Med. Chem. Lett. 25(3): 721-727 (2015).

Co-Crystallization of hGALK1m with Inhibitors

Since human GALK1 (hGALK1; SEQ ID NO:3) has been co-crystallized with AMP-PNP and galactose previously (PDB 1WUU), we attempted to reproduce this by following the published methods and conditions, but without success. Holden et al., Cell. Mol. Life Sci. 61(19-20): 2471-2484 (2004). One of the major obstacles encountered was that the wild type hGALK1 aggregated at higher concentrations. The highest concentration we could achieve without aggregation was 6 to 7 mg/mL. In order to improve the solubility of hGALK1, we performed the surface entropy reduction analysis and test, and identified Lys252 and Glu253 as potential high surface entropy residues. Substituting these two residues with alanine residues resulted in significant improvement of the solubility of the protein, from 6 to 7 mg/mL for the wild type protein to 18 mg/mL for the K252A and E253A modified protein (hGALK1m) (SEQ ID NO: 3). The introduction of these two mutations to the protein did not affect either the K_{M} for ATP or the K_{M} for galactose. Further, these mutations did not affect the Vₘₐₓ of the enzyme (data not shown). Further characterization also showed that previously identified hGALK1 inhibitors also inhibited hGALK1m with the same potency as for the wild type enzyme (data not shown). We first successfully co-crystallized hGALK1m with galactose and ADP and used these crystals as seeds for co-crystallization of hGALK1m and its inhibitors.

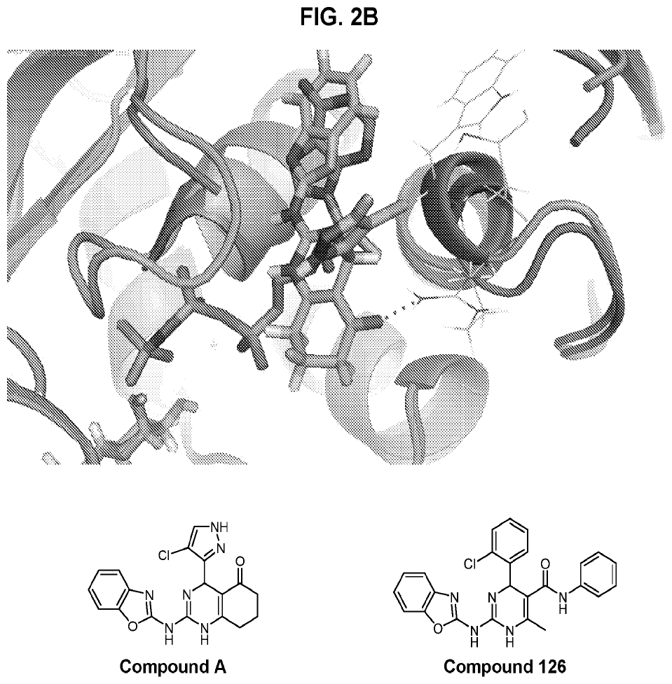

The first compound co-crystallized with hGALK1m was Compound A (FIG. 2), which was identified and characterized previously as one of the best ATP competitive inhibitors for hGALK1. The overall structure of hGALK1m-Compound A complex was nearly identical to the original human GALK1 crystal structure (PDB Accession No. 1WUU; SEQ ID NO: 4) (FIG. 2A). The Root Mean Square Deviation (RMSD) between the two structures was 1.23 Å (0.123 nm). This further confirmed that the surface entropy reduction mutations only increased the solubility of the protein and did not affect any other structural properties of the enzyme. Even the conformation of the loop, where the two mutations were located, did not change (FIG. 2A, yellow circle). Our structure had greater resolution (2.1 Å) compared to the 1WUU structure (2.5 Å), and a well-defined structure for Ser 230 and Leu 231, which were missing in the 1WUU structure. Previous studies identified Compound A as an ATP competitive inhibitor for human GALK1, and our structure clearly showed that Compound A occupied the ATP binding pocket of the enzyme. The benzoxazole ring of Compound A occupied the area where the adenine ring of ATP binds. Because the electron densities of oxygen and nitrogen are difficult to differentiate, we could not determine the orientation of the benzoxazole ring from this structure, and the orientation was arbitrarily assigned (FIG. 2B). The ketone group formed a hydrogen bond with Arg 105, and the spiro group occupied a pocket surrounded by Arg 105, Trp 106 and Asp 83 (FIG. 2B).

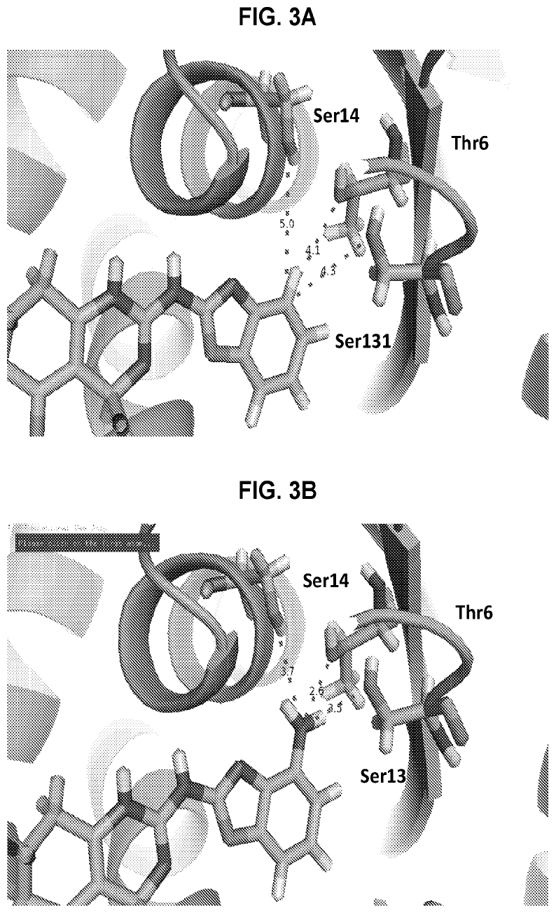

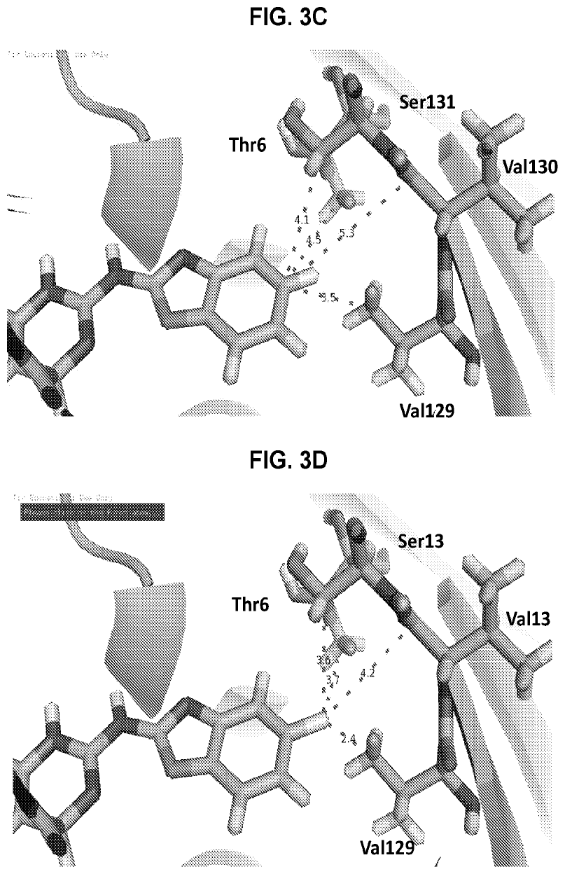

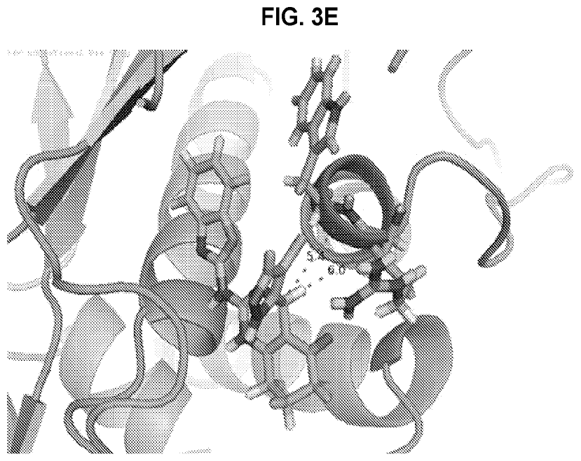

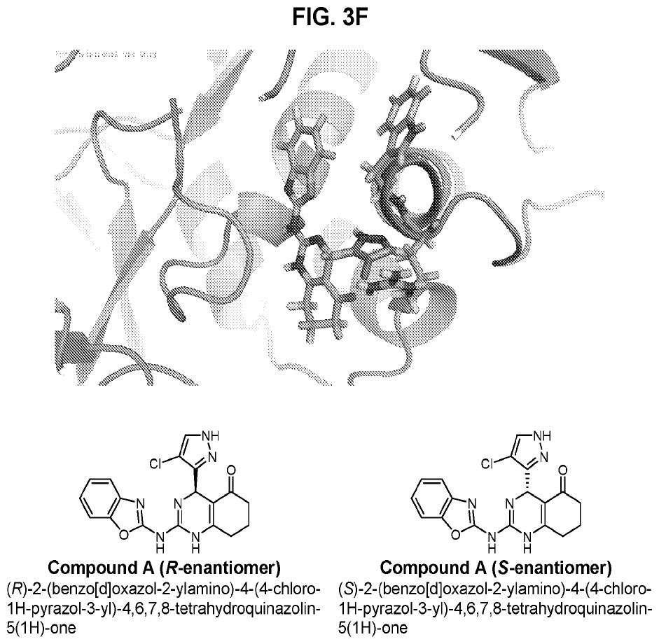

Study of Benzoxazole Ring-Binding Pocket The detailed study of the co-crystal structure revealed two interesting pockets near the benzoxazole ring. The first pocket was made up of the hydroxyl groups of Ser 144, Thr 61 and Ser 131, surrounding the 4^{th} or 7^{th} carbon atom of the benzoxazole ring, depending on the ring orientation (FIG. 3A). The oxygen atoms of these hydroxyl groups of the amino acids were located about 4.1 to 5 Å away from the 4^{th} or 7^{th} carbon atom. We hypothesized that if an amide group was added to this pocket, it could potentially form hydrogen bonds with the surrounding hydroxyl groups. Indeed, when we added an amide group to the structure model, the nitrogen atom of the amide group was only 2.6 Å, 3.5 Å, and 3.7 Å, respectively, away from the oxygen atoms of the hydroxyl groups of Thr 61, Ser 131 and Ser 144, respectively (FIG. 3B). Thr 61 had the highest possibility of forming a hydrogen bond with the added amide group because it had the shortest distance and optimum angle for interacting with the putative amide moiety.

The second pocket is a small hydrophobic pocket comprising the γ carbon of Thr 61, the β carbon of Ser 131, the backbone carbon of Val 130, and the β carbon of Val 129 (FIG. 3C). These atoms were only 3.5 to 5.3 Å away from the C5 of the benzoxazole ring (FIG. 3C); accordingly, a methyl group at the C5 position would likely be too bulky. However, substitution of the C5 hydrogen atom with a fluorine, chlorine, or bromide would theoretically fill the space and form halogen-hydrophobic interactions (FIG. 3D).

Because Compound A has a chiral center, we interrogated whether the hGALK1m protein prefers the R- or S-enantiomer. The crystal structure clearly showed that only the S-enantiomer was co-crystalized with the protein (FIG. 3E). The chiral carbon atom was only 5.4 Å and 6.0 Å away from backbone atoms of Trp 106 and Arg 105, respectively (FIG. 3E). If conversely the R-enantiomer was incorporated, the pyrazole group of Compound A would collide with the α-helix containing Trp 106 and Arg 105 (FIG. 3F).

Optimization of hGALK1m Inhibitors Benzene and several of its derivatives, including isonicotinic acid, and benzoic acid at both the para and meta positions, were tested as amide group substituents (Compounds 137 to 143, Table 17). Among them, isonicotinic acid substitution demonstrated the highest potency, with an IC₅₀ of 0.97 μM (Compound 137). Also, several thiadiazole, thiazole, thiadiazole, and imidazole derivatives were tested as amide substituents (Compounds 205 to 173 and Compounds 233 to 230, Table 17). The compound with methylimidazole meta-amide substitution demonstrated the best activity among this group of compounds, with an IC₅₀ of 0.77 μM (Compound 233). In addition, larger groups such as benzimidazole were tested (Compound 234, Table 17), however, these moieties significantly impaired the potency. Due to solubility concerns, and the relatively small difference in IC₅₀ values between the compounds with isonicotinic acid- and methylimidazole-amide substitution, Compound 137 was selected for further optimization.

### Benzoxazole Ring Optimization

As mentioned above, there were two pockets surrounding the benzoxazole ring of Compound A, which might accommodate an amine substituent for hydrogen bond formation at the C4 or C7 position of the benzoxazole ring, and a fluorine substituent for halogen-hydrophobic interactions at the C5 or C6 position of the ring, depending on the orientation of the benzoxazole ring. We designed two fluorine substitutions at either C5 or C6 position (Compounds 1 and 22, Table 18), accounting for both possibilities, to test the hypothesis that adding a halogen atom might form halogen-hydrophobic interactions with the hydrophobic pocket. Interestingly, Compound 1, which was substituted at C6 position, showed nearly 4-fold increase in potency. However, the inhibitory properties of Compound 22, which was substituted at C5 position, decreased by 3.6-fold. These results validated our hypothesis that fluorine substitution at the benzoxazole ring could enable halogen-hydrophobic interactions with hGALK1m, and set the orientation of the benzoxazole ring, with the 09 atom sited closer to the two identified pockets and the N2 atom located at the other side (our initial arbitrary assignment of the ring orientation was in the wrong way). In addition, because the orientation of the benzoxazole ring was determined, we tested amine substitution at the C7 position of the ring, which resulted in Compound 3 (Table 18). The IC₅₀ of Compound 3 improved 13-fold compared with the parent compound, Compound 137. This confirmed another hypothesis that substituting an amine group at the C7 position of the benzoxazole ring would potentially allow the compound to form hydrogen bonds with Thr 61, Ser 131, or Ser 144. Because the potency of Compound 3 only improved 13-fold, it is likely that only one hydrogen bond was formed between Compound 3 and the protein.

### Further SAR for the Benzoxazole Ring

Since C6 fluorine substitution enabled halogen-hydrophobic interactions with the protein, we further substituted with other halogen groups at the same position in order to see if the other halogens give similar results. C6 chlorine substitution resulted in Compound 14 and decreased potency more than 2-fold, which implied this pocket could not accommodate halogen atoms larger than fluorine. Similarly, methyl substitution at the C6 position resulted in nearly a 7-fold decrease of the potency (Compound 6, Table 18), which further confirmed this pocket only had limited accessibility.

We performed a similar exploration of various substituents at the C7 position (Table 18). Amide substitution was proven to be optimum substitution at this position, which well correlated with our structure studies and simulation. Methyl substitution improved the potency by 2.6-fold (Compound 15, Table 17). Chlorine or fluorine substitutions did not change the potency of the compounds (Compounds 17 and 10, Table 18). Other larger group substitutions, such as trifluoromethyl group, trifluoromethanol group, and methanol group, all greatly compromised the potencies (Compounds 22, 21 and 16, Table 18).

In addition, we explored various functional groups at the C4 and C5 positions, including halogen, methyl, methanol, and amine groups. With the exception of the methyl substitution at C5 position, wherein such substitution slightly improved the potency of the compound (Compound 4, Table 18), all other substitutions compromised the compounds' potency against hGALK1m (Compounds 11 to 18, and 5 to 13, Table 18).

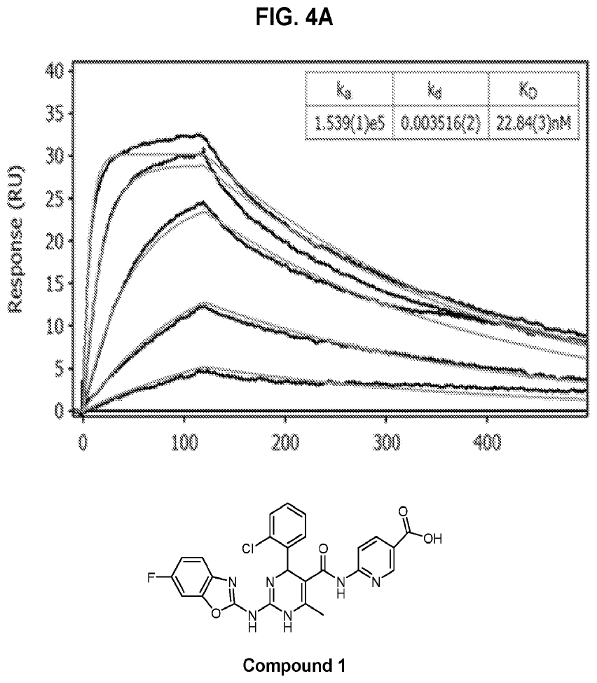

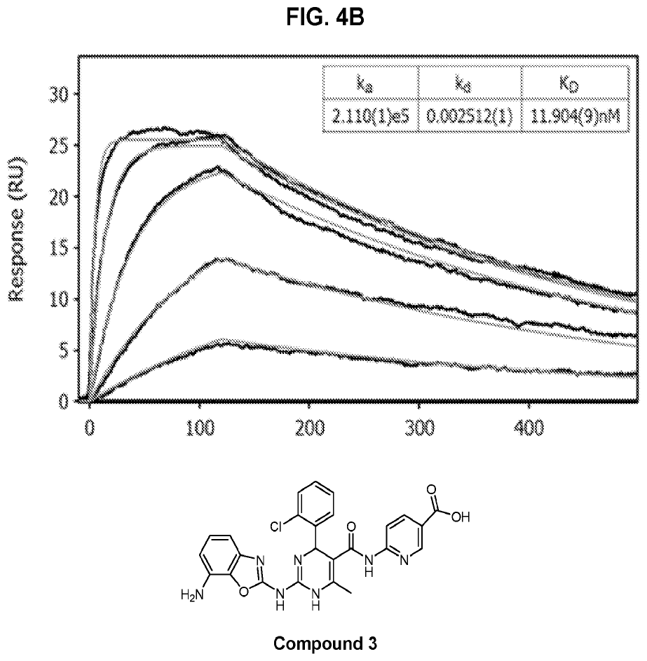

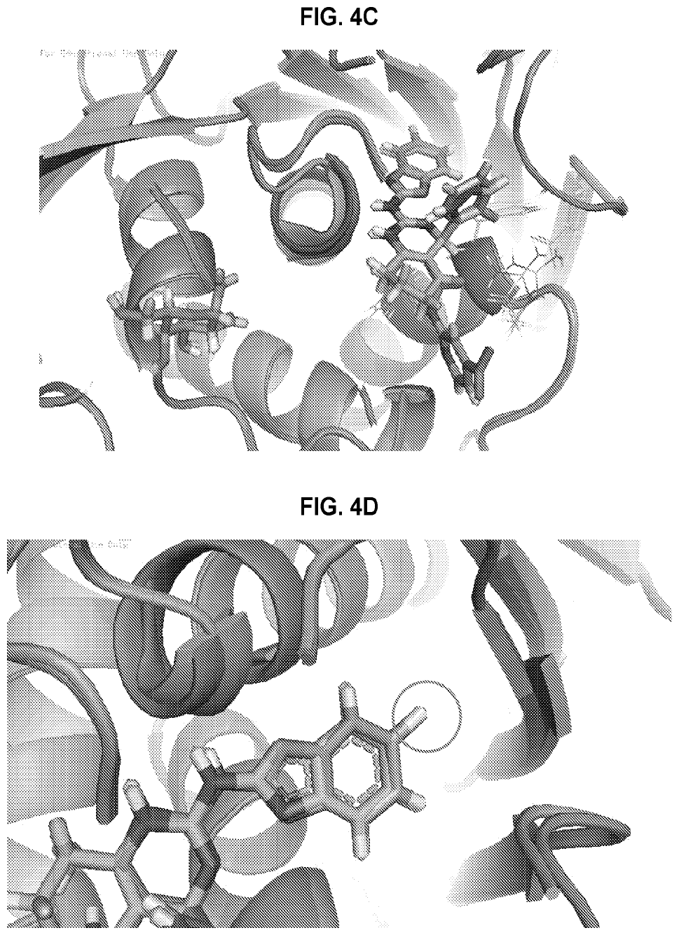

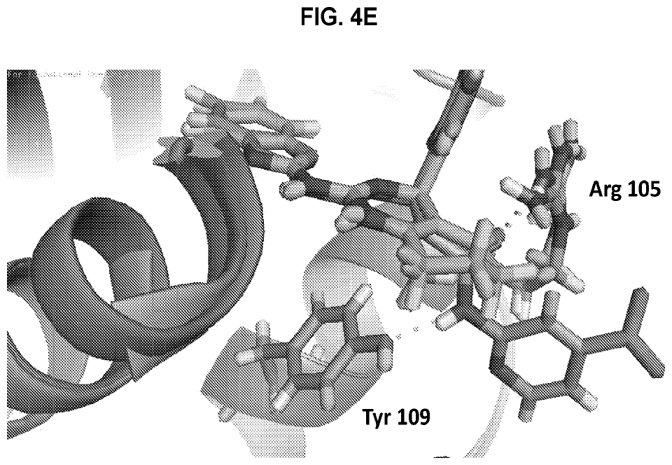

Further characterization of the two lead compounds thus far, Compound 1 and Compound 3, with surface plasma resonance (SPR), confirmed that these compounds bind to hGALK1m tightly. Their K_{D}s were 23 nM and 12 nM respectively at 4° C. (FIGS. 4A and 3B). We attempted to co-crystalize these two compounds with hGALK1m, but only succeeded with Compound 1. The co-crystal structure showed Compound 1 also binds to the ATP-binding site of the enzyme and its binding pose well-overlapped with Compound A (FIG. 4C). Even with a fluorine substitution at C6 position, the benzoxazole ring of Compound 1 absolutely overlapped with that of Compound A (FIG. 4D). The aryl rings of Compounds 1 and A also showed significant overlap, and occupied the same pocket (FIG. 4C). The decahydroquinazoline ring was opened in Compound 1, but the hydrogen bond between ketone group and Arg 105 remained intact, and Arg 105 moved closer to the ketone group in the crystal structure with Compound 1 (FIG. 4E). In addition, the amide's nitrogen atom formed a hydrogen bond with Tyr 109 (FIG. 4E), which could clarify why substituting the closed ring with opened amide structure improves the compound's activity. The isonicotinic acid moiety extended outside of the enzyme's active center and was solvent exposed (FIG. 4C). As the crystallography data demonstrated that the benzoxazole ring's binding conformation did not change upon substitution of fluorine at C6 position and the C7 pocket left untouched by Compound 1 (FIG. 4D), we proposed that combined C6 fluorine substitution and C7 amine substitution in one molecule would result in a more potent hGALK1m inhibitor.

### Aryl Group Optimization

Next, we focused on optimizing aryl group of the compound, using Compound 1 as the parent compound. Substituting chlorine with bromine had little impact on the potency of compound (Compound 75, Table 19). Also, adding halogen substitutions at the para site of the benzene ring had little effect on the potency of the compounds (Compounds 45 and 47, Table 19). Adding a methyl substituent at the para site improved the potency by 2-fold (Compound 50, Table 19), while incorporating a methanol group at the same position had little effect (Compound 46, Table 19). Halogen or alkylhalogen substitutions, such as chlorine, fluorine, or trifluoromethyl, at the meta site compromised the potency of compounds (Compounds 48, 49, 51, and 77, Table 19). Finally, substituting a chlorobenzene group with a phenylethyleneimine group and a benzenesulphonamide group all significantly impaired the potency (Compounds 76 and 78, Table 19).

In addition, as indicated in the co-crystal structure, that hGALK1m preferred the S-form enantiomer of the compounds, we isolated the two enantiomers of Compound 194 and tested each of their individual activities, as well as the racemic mixture's activity, against the enzyme. Indeed, the S-enantiomer was significantly more potent than both the R-enantiomer and the racemic mixture. The IC₅₀ of the S-enantiomer of Compound 194 was 300 nM, the IC₅₀ of the R-enantiomer was 15 μM, and the IC₅₀ of the racemic mixture was 970 nM. These results indicated enantiomer separation could be an effective way improving the potencies of this series of compounds against hGALK1m.

Further Optimization of hGALK1m Inhibitors

In our amide optimization mentioned above, both isonicotinic acid- and methylimidazole-substituted compounds had sub-micromolar potencies, and the methylimidazole-substituted compound was slightly more potent than isonicotinic acid-substituted compound (Table 17). However, the isonicotinic acid-substituted compound was selected for its improved solubility. Yet, later studies found that isonicotinic acid can cause permeability problems for the compounds (Table 17). Consequently, methylpyrazole and ethylpyrazole moieties were selected to replace the isonicotinic acid moiety for our final optimization. The substitution of the isonicotinic acid moiety with a methylpyrazole, and the replacement of the aryl group with a methyl group at the para position, for Compound 3 resulted in Compound 83, which demonstrated superior potency (IC₅₀ of 47 nM, Table 20). The S-enantiomer of Compound 83 was even more potent (IC₅₀ of 29 nM). Fluorine substitution at the C6 position of Compound 83 resulted in Compound 126 with an IC₅₀ of 14 nM. Substituting the methylpyrazole group with an ethylpyrazole group generated Compound 125, which was slightly less potent (IC₅₀ of 23 nM). The S-enantiomer of Compound 83 and racemic mixture of Compounds 126, 125 were the most potent hGALK1m inhibitors to-date. In addition, based on our structural studies and experiences with Compound 194 and Compound 83, we predicted that the S-enantiomer of Compounds 126 and 125 would have even better potency. With successful isolation of the S-enantiomer of Compound 126, the IC₅₀ could fall below 10 nM.

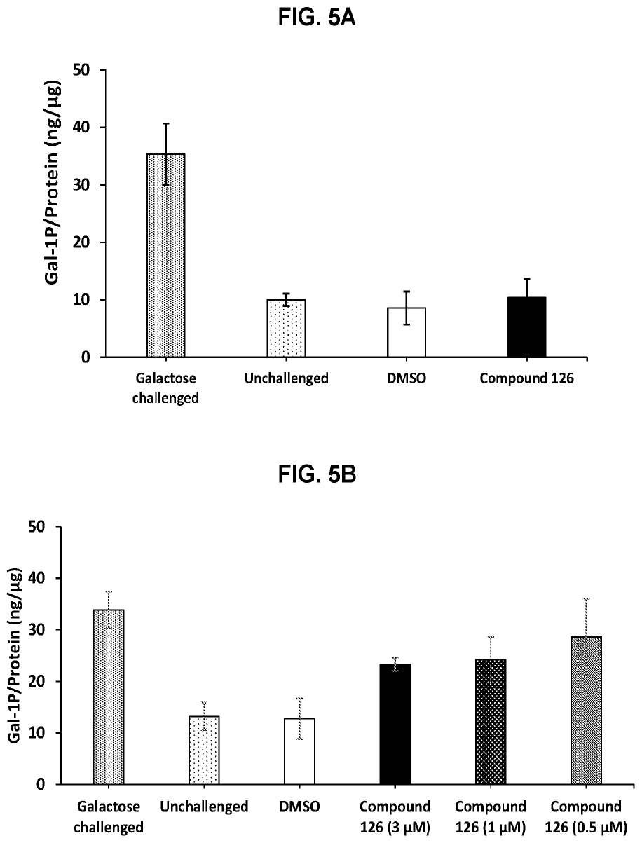

Finally, Compounds 126 and 125 were tested for their hGALK1m inhibitory potency on patient fibroblasts. Pre-treating the patient cells with Compounds 126 at 10 μM could completely prevent gal-1P accumulation inside the cell, while Compound 125 could inhibit about 70% of gal-1P accumulation at 10 μM (FIG. 5).

Even though newborn screening programs and removal galactose from patients' diets save them from neonatal lethality, there is currently no approved, satisfactory treatment for the long-term complications associated with classic galactosemia. The pathophysiological mechanisms have been controversial for a long time, and it is unclear whether galactosemia is caused by accumulation of galactitol, galactonate, gal-1P, or a deficiency of UDP-galactose. Patients with inherited deficiency of galactokinase give some indications for the answer to this question. These patients accumulate galactose and galactitol like the GALT-deficient patients, but they do not build up gal-1P. Segal and Berry, The Metabolic Basis of Inherited Diseases, Scriver et al. Eds., McGraw-Hill: New York, 967-1000 (1995); Gitzelmann et al., Eur. J. Clin. Invest. 4(2): 79-84 (1974). Except cataracts, they often do not experience any of the long-term complications like the patients with classic galactosemia. These facts build a strong case for the link between gal-1P accumulation and chronic complications of classic galactosemia. Segal and Berry, The Metabolic Basis of Inherited Diseases, Scriver et al. Eds., McGraw-Hill: New York, 967-1000 (1995); Gitzelmann et al., Eur. J. Clin. Invest. 4(2): 79-84 (1974); Gitzelmann, J. Pediatr. 87(6 Pt 1): 1007-1008 (1975); Bosch et al., J. Inherit. Metab. Dis. 25(8): 629-364 (2002). A recent drosophila model argued gal-1P accumulation is not the main reason for these complications because GALK/GALT double knockout did not rescue the larvae. Daenzer et al., Dis. Model Mech. 9(11): 1375-1382 (2016). In that study, simply knocking out GALK was not benign and phenocopied GALT null animal, which was quite different from what commonly manifested in human patients. In addition, another drosophila model of classic galactosemia published earlier demonstrated that galactokinase gene was a genetic modifier of the neurological defects of this model, and co-removal of dGALK corrected these defects, which were well correlated with human observations and yeast results. Jumbo-Lucioni et al., Dis. Model Mech. 7(12): 1365-1378 (2014).

Even though animal model studies generated controversial results, most evidences pointed to gal-1P as the major cause for the chronic complications and GALK1 remains one of the major therapeutic targets. Previously, multiple compounds and chemotypes have been identified as hGALK1 inhibitors using high throughput screening or virtual screening. Wierenga et al., J. Biomol. Screen. 13(5): 415-423 (2008); Hu et al., J. Comput Aided Mol. Des. 33(4): 405-417 (2019); Liu et al., Bioorg. Med. Chem. Lett. 25(3): 721-727 (2015). However, the IC₅₀ values for these compounds were in micromolar range. Compound A is one of the compounds identified by these studies, and it represents a unique chemotype with exceptional selectivity against hGALK1. In order to further improve the potency of this chemotype, we employed a structure-based optimization strategy. Co-crystallization of Compound A with hGALK1m revealed two interesting pockets close to the C6 and C7 positions of the benzoxazole ring of Compound A. Simulation studies indicated that an amine substitution at the C7 site and a halogen substitution at the C6 site would form favorable interactions with the protein. SAR studies confirmed the simulation results. The C7 amine substitution exhibited the strongest improvement among all the modifications with a 13-fold increase in the IC₅₀. The C6 fluorine substitution also substantially improved the IC₅₀ by nearly 4-fold. More importantly, additional crystallography studies confirmed these two modifications were independent of each other, and that combining them into one molecule was possible, which resulted in an even more potent compound. In addition, fine tuning of both the amide and aryl groups resulted in a 7-fold and a 2-fold improvement of the IC₅₀, respectively. Combining all of these modifications resulted in Compounds 126 and 125, which, as racemic mixtures had an IC₅₀ of 14 nM and 22 nM, respectively. The structural studies revealed that hGALK1m preferred the S-enantiomers of this chemotype, which was proved in later studies. We believe isolating the S-enantiomer of Compounds 126 and 125 would result in hGALK1 inhibitors with potency under 10 nM. Testing Compounds 126 and 125 on fibroblasts from galactosemia patients showed that Compound 126 could prevent 100% of gal-1P accumulation at 10 μM and that Compound 125 could prevent 70% of gal-1P accumulation at the same concentration. Collectively, Compound 126 and 125 are candidates for further development as novel therapeutic agents for galactosemia patients.

TABLE 17
Amide Group Optimization

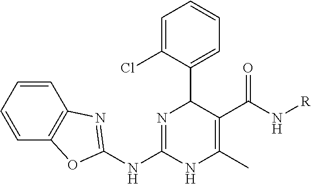

| R | ID | IC₅₀ | Efficacy | ADME |
|---|---|---|---|---|
| 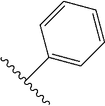 [SMILES: *c1ccccc1] | 137 | 6.9 | 57% | M, P, S |
| 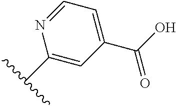 [SMILES: *c1cc(C(=O)O)ccn1] | 194 | 0.97 | 88% | M, P, S |
| 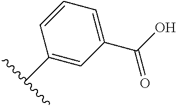 [SMILES: *c1cccc(C(=O)O)c1] | 142 | 3.1 | 96% | M, P, S |
| 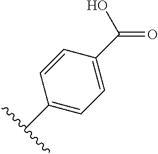 [SMILES: *c1ccc(C(=O)O)cc1] | 143 | 1.4 | 100% | M, P, S |
| 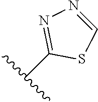 [SMILES: *c1nncs1] | 205 | 3.4 | 96% | M, P, S |
| 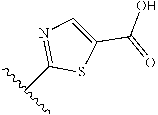 [SMILES: *c1ncc(C(=O)O)s1] | 199 | 1.7 | 91% | M, P, S |
| 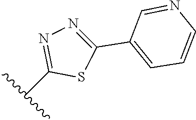 [SMILES: *c1nnc(-c2cccnc2)s1] | 8 | 3.1 | 94% | M, P, S |
| 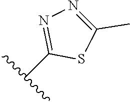 [SMILES: *c1nnc(C)s1] | 173 | 3.9 | 95% | M, P, S |
| 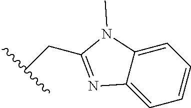 [SMILES: *Cc1nc2ccccc2n1C] | 234 | NA | 48% | M, P, S |
| 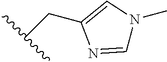 [SMILES: *Cc1cn(C)cn1] | 233 | 0.77 | 90% | M, P, S |
| 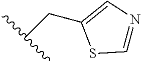 [SMILES: *Cc1cncs1] | 232 | 1.2 | 84% | M, P, S |
| 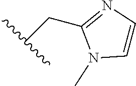 [SMILES: *Cc1nccn1C] | 231 | 11 | 94% | M, P, S |
| 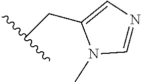 [SMILES: *Cc1cncn1C] | 230 | 1.4 | 98% | M, P, S |

M: rat microsomal stability (M = t_{1/2} < 10 min, M = t_{1/2} = 10-30 min, M = t_{1/2} > 30 min)
P: PAMPA permeability (P = <10 × 10⁻⁶ cm/s, P = 10-100 × 10⁻⁶ cm/s, P = >100 × 10⁻⁶ cm/s)
S: aqueous kinetic solubility (S = <10 mg/mL, S = 10-40 mg/mL, S = >40 mg/mL)

TABLE 18
Benzoxazole Group Optimization

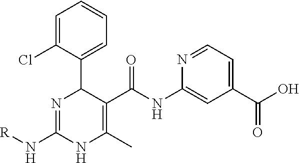

| | | | | Max | |
|---|---|---|---|---|---|
| | | | | Response | |
| R | ID | IC₅₀ | | (%) | ADME |
| 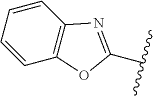 [SMILES: *c1nc2ccccc2o1] | 137 | 970 | nM | 100% | M, P, S |
| 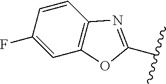 [SMILES: *c1nc2ccc(F)cc2o1] | 1 | 260 | nM | 100% | M, P, S |
| 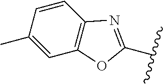 [SMILES: *c1nc2ccc(C)cc2o1] | 6 | 6.9 | μM | 100% | M, P, S |
| 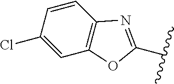 [SMILES: *c1nc2ccc(Cl)cc2o1] | 14 | 2.25 | μM | 100% | M, P, S |
| 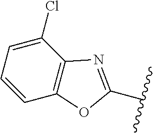 [SMILES: *c1nc2c(Cl)cccc2o1] | 11 | 5.8 | μM | 85% | M, P, S |
| 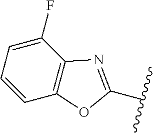 [SMILES: *c1nc2c(F)cccc2o1] | 8 | 3.3 | μM | 100% | M, P, S |
| 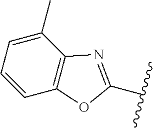 [SMILES: *c1nc2c(C)cccc2o1] | 18 | 5.5 | μM | 100% | M, P, S |
| 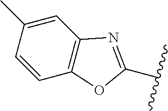 [SMILES: *c1nc2cc(C)ccc2o1] | 4 | 611 | nm | 100% | M, P, S |
| 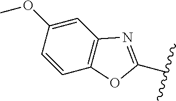 [SMILES: *c1nc2cc(OC)ccc2o1] | 5 | 2 | μM | 100% | M, P, S |
| 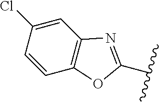 [SMILES: *c1nc2cc(Cl)ccc2o1] | 9 | 1.7 | μM | 100% | M, P, S |
| 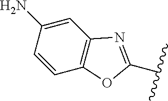 [SMILES: *c1nc2cc(N)ccc2o1] | 12 | 5.5 | μM | 100% | M, P, S |
| 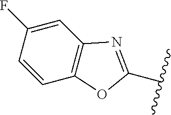 [SMILES: *c1nc2cc(F)ccc2o1] | 13 | 3.6 | μM | 100% | M, P, S |
| 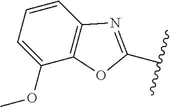 [SMILES: *c1nc2cccc(OC)c2o1] | 22 | >15 | μM | 80% | M, P, S |
| 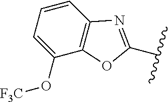 [SMILES: *c1nc2cccc(OC(F)(F)F)c2o1] | 21 | >15 | μM | 88% | M, P, S |
| 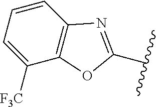 [SMILES: *c1nc2cccc(C(F)(F)F)c2o1] | 16 | >15 | μM | 100% | M, P, S |
| 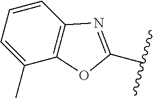 [SMILES: *c1nc2cccc(C)c2o1] | 15 | 365 | nM | 100% | M, P, S |
| 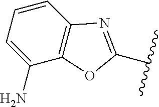 [SMILES: *c1nc2cccc(N)c2o1] | 3 | 77 | nM | 100% | M, P, S |
| 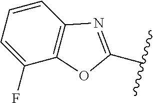 [SMILES: *c1nc2cccc(F)c2o1] | 17 | 1 | μM | 100% | M, P, S |
| 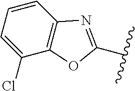 [SMILES: *c1nc2cccc(Cl)c2o1] | 10 | 1 | μM | 100% | M, P, S |

M: rat microsomal stability (M = t_{1/2} < 10 min, M = t_{1/2} = 10-30 min, M = t_{1/2} > 30 min)
P: PAMPA permeability (P = <10 × 10⁻⁶ cm/s, P = 10-100 × 10⁻⁶ cm/s, P = >100 × 10⁻⁶ cm/s)
S: aqueous kinetic solubility (S = <10 mg/mL, S = 10-40 mg/mL, S = >40 mg/mL)

TABLE 19
Aryl Group Optimization

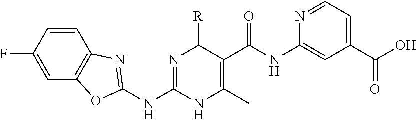

| | | | | Max Re- | |
|---|---|---|---|---|---|
| | | | | sponse | |
| R | ID | IC₅₀ | | (%) | ADME |
| 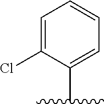 [SMILES: *c1ccccc1Cl] | 1 | 260 | nM | 100% | M, P, S |
| 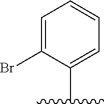 [SMILES: *c1ccccc1Br] | 75 | 235 | nM | 100% | M, P, S |
| 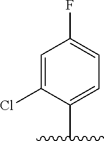 [SMILES: *c1ccc(F)cc1Cl] | 45 | 258 | nM | 100% | M, P, S |
| 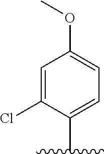 [SMILES: *c1ccc(OC)cc1Cl] | 46 | 300 | nM | 100% | M, P, S |
| 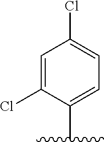 [SMILES: *c1ccc(Cl)cc1Cl] | 47 | 345 | nM | 85% | M, P, S |
| 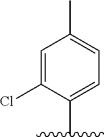 [SMILES: *c1ccc(C)cc1Cl] | 50 | 136 | nM | 100% | M, P, S |
| 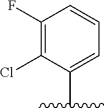 [SMILES: *c1cccc(F)c1Cl] | 48 | 1.8 | μM | 100% | M, P, S |
| 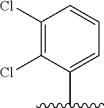 [SMILES: *c1cccc(Cl)c1Cl] | 49 | 3.6 | μM | 100% | M, P, S |
| 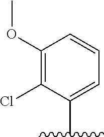 [SMILES: *c1cccc(OC)c1Cl] | 52 | 5.4 | μM | 100% | M, P, S |
| 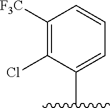 [SMILES: *c1cccc(C(F)(F)F)c1Cl] | 51 | 2.4 | μM | 97% | M, P, S |
| 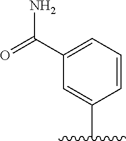 [SMILES: *c1cccc(C(N)=O)c1] | 76 | 3.8 | μM | 90% | M, P, S |
| 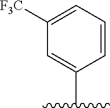 [SMILES: *c1cccc(C(F)(F)F)c1] | 77 | >15 | μM | 90% | M, P, S |
| 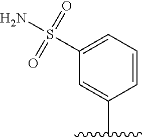 [SMILES: *c1cccc(S(N)(=O)=O)c1] | 78 | >15 | μM | 75% | M, P, S |

M: rat microsomal stability (M = t_{1/2} < 10 min, M = t_{1/2} = 10-30 min, M = t_{1/2} > 30 min)
P: PAMPA permeability (P = <10 × 10⁻⁶ cm/s, P = 10-100 × 10⁻⁶ cm/s, P = >100 × 10⁻⁶ cm/s)
S: aqueous kinetic solubility (S = <10 mg/mL, S = 10-40 mg/mL, S = >40 mg/mL)

TABLE 20
SAR Optimized Compounds

| | |
|---|---|
| 47 nM 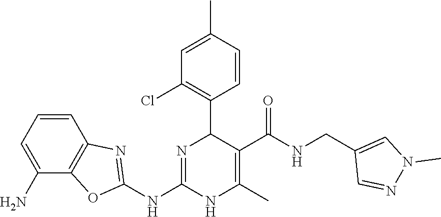 [SMILES: CC1=C(C(=O)NCc2cnn(C)c2)C(c2ccc(C)cc2Cl)N=C(Nc2nc3cccc(N)c3o2)N1] | Compound 83 |
| 14 nM 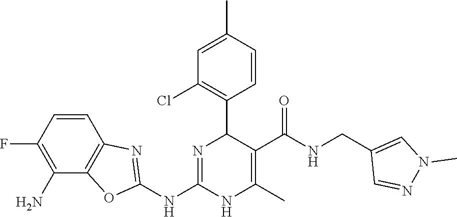 [SMILES: CC1=C(C(=O)NCc2cnn(C)c2)C(c2ccc(C)cc2Cl)N=C(Nc2nc3ccc(F)c(N)c3o2)N1] | Compound 126 |
| 22 nM 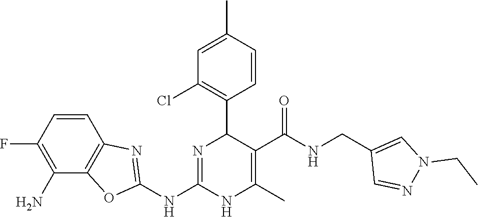 [SMILES: CCn1cc(CNC(=O)C2=C(C)NC(Nc3nc4ccc(F)c(N)c4o3)=NC2c2ccc(C)cc2Cl)cn1] | Compound 125 |

Other compounds described herein are listed in Table 21.

TABLE 21

| | |
|---|---|
| 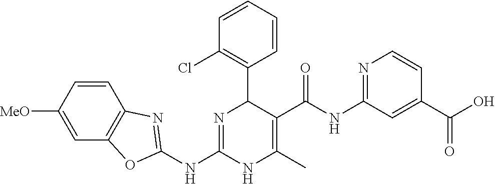 [SMILES: COc1ccc2nc(NC3=NC(c4ccccc4Cl)C(C(=O)Nc4cc(C(=O)O)ccn4)=C(C)N3)oc2c1] 2-(4-(2-chlorophenyl)-2-((6- methoxybenzo[d]oxazol-2-yl)amino)-6-methyl-1,4- dihydropyrimidine-5-carboxamido)isonicotinic acid | (285) |
| 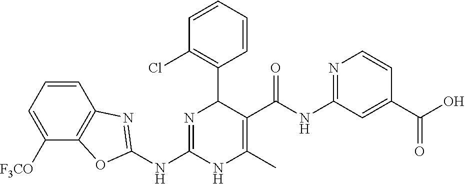 [SMILES: CC1=C(C(=O)Nc2cc(C(=O)O)ccn2)C(c2ccccc2Cl)N=C(Nc2nc3cccc(OC(F)(F)F)c3o2)N1] 2-(4-(2-chlorophenyl)-6-methyl-2-((7- (trifluoro(oxo)-λ⁶-methyl)benzo[d]oxazol-2- yl)amino)-1,4-dihydropyrimidine-5- carboxamido)isonicotinic acid | (286) |
| 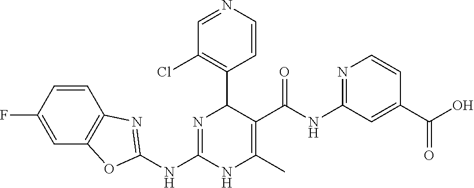 [SMILES: CC1=C(C(=O)Nc2cc(C(=O)O)ccn2)C(c2ccncc2Cl)N=C(Nc2nc3ccc(F)cc3o2)N1] 2-(4-(3-chloropyridin-4-yl)-2-((6- fluorobenzo[d]oxazol-2-yl)amino)-6-methyl-1,4- dihydropyrimidine-5-carboxamido)isonicotinic acid | (287) |
| 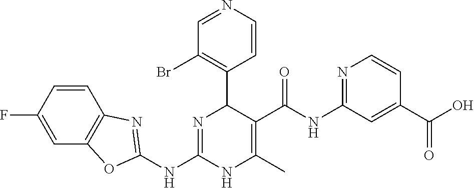 [SMILES: CC1=C(C(=O)Nc2cc(C(=O)O)ccn2)C(c2ccncc2Br)N=C(Nc2nc3ccc(F)cc3o2)N1] 2-(4-(3-bromopyridin-4-yl)-2-((6- fluorobenzo[d]oxazol-2-yl)amino)-6-methyl-1,4- dihydropyrimidine-5-carboxamido)isonicotinic acid | (288) |
| 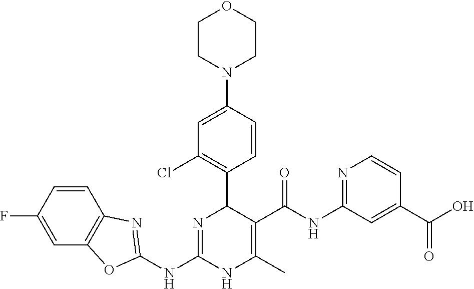 [SMILES: CC1=C(C(=O)Nc2cc(C(=O)O)ccn2)C(c2ccc(N3CCOCC3)cc2Cl)N=C(Nc2nc3ccc(F)cc3o2)N1] 2-(4-(2-chloro-4-morpholinophenyl)-2-((6- fluorobenzo[d]oxazol-2-yl)amino)-6-methyl-1,4- dihydropyrimidine-5-carboxamido)isonicotinic acid | (289) |
| 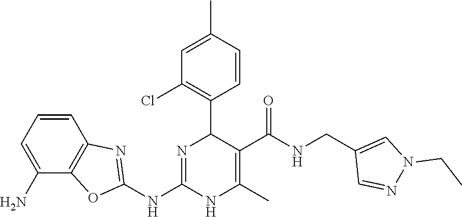 [SMILES: CCn1cc(CNC(=O)C2=C(C)NC(Nc3nc4cccc(N)c4o3)=NC2c2ccc(C)cc2Cl)cn1] 2-((7-aminobenzo[d]oxazol-2-yl)amino)-4-(2- chloro-4-methylphenyl)-N-((1-ethyl-1H-pyrazol- 4-yl)methyl)-6-methyl-1,4-dihydropyrimidine-5- carboxamide | (290) |
| 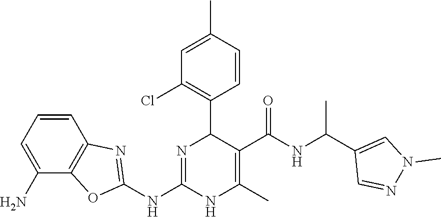 [SMILES: CC1=C(C(=O)NC(C)c2cnn(C)c2)C(c2ccc(C)cc2Cl)N=C(Nc2nc3cccc(N)c3o2)N1] 2-((7-aminobenzo[d]oxazol-2-yl)amino)-4-(2- chloro-4-methylphenyl)-6-methyl-N-(1-(1-methyl- 1H-pyrazol-4-yl)ethyl)-1,4-dihydropyrimidine-5- carboxamide | (291) |
| 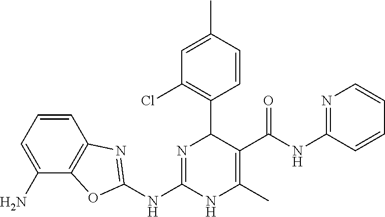 [SMILES: CC1=C(C(=O)Nc2ccccn2)C(c2ccc(C)cc2Cl)N=C(Nc2nc3cccc(N)c3o2)N1] 2-((7-aminobenzo[d]oxazol-2-yl)amino)-4-(2- chloro-4-methylphenyl)-6-methyl-N-(pyridin-2- yl)-1,4-dihydropyrimidine-5-carboxamide | (292) |
| 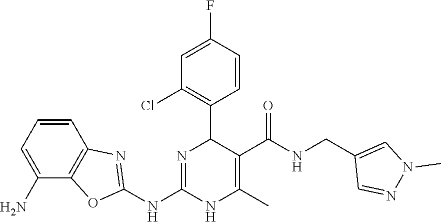 [SMILES: CC1=C(C(=O)NCc2cnn(C)c2)C(c2ccc(F)cc2Cl)N=C(Nc2nc3cccc(N)c3o2)N1] 2-((7-aminobenzo[d]oxazol-2-yl)amino)-4-(2- chloro-4-fluorophenyl)-6-methyl-N-((1-methyl-1H- pyrazol-4-yl)methyl)-1,4-dihydropyrimidine-5- carboxamide | (293) |
| 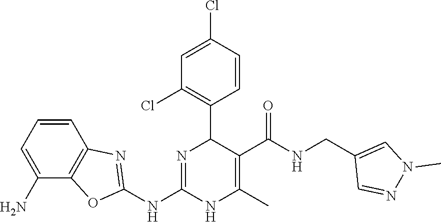 [SMILES: CC1=C(C(=O)NCc2cnn(C)c2)C(c2ccc(Cl)cc2Cl)N=C(Nc2nc3cccc(N)c3o2)N1] 2-((7-aminobenzo[d]oxazol-2-yl)amino)-4-(2,4- dichlorophenyl)-6-methyl-N-((1-methyl-1H- pyrazol-4-yl)methyl)-1,4-dihydropyrimidine-5- carboxamide | (294) |
| 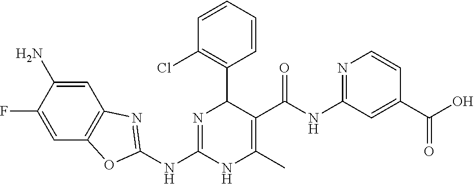 [SMILES: CC1=C(C(=O)Nc2cc(C(=O)O)ccn2)C(c2ccccc2Cl)N=C(Nc2nc3cc(N)c(F)cc3o2)N1] 2-(2-((5-amino-6-fluorobenzo[d]oxazol-2-yl)amino)- 4-(2-chlorophenyl)-6-methyl-1,4-dihydropyrimidine- 5-carboxamido)isonicotinic acid | (295) |
| 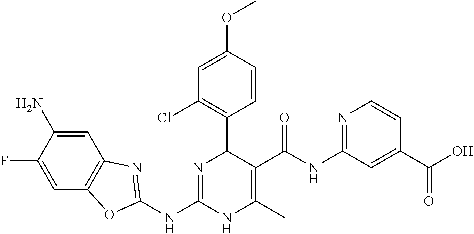 [SMILES: COc1ccc(C2N=C(Nc3nc4cc(N)c(F)cc4o3)NC(C)=C2C(=O)Nc2cc(C(=O)O)ccn2)c(Cl)c1] 2-(2-((5-amino-6-fluorobenzo[d]oxazol-2- yl)amino)-4-(2-chloro-4-methoxyphenyl)-6- methyl-1,4-dihydropyrimidine-5- carboxamido)isonicotinic acid | (296) |
| 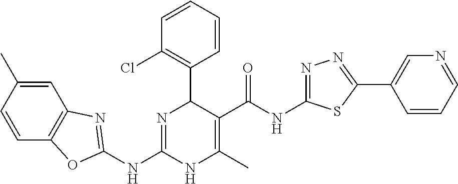 [SMILES: CC1=C(C(=O)Nc2nnc(-c3cccnc3)s2)C(c2ccccc2Cl)N=C(Nc2nc3cc(C)ccc3o2)N1] 4-(2-chlorophenyl)-6-methyl-2-((5- methylbenzo[d]oxazol-2-yl)amino)-N-(5-(pyridin-3- yl)-1,3,4-thiadiazol-2-yl)-1,4-dihydropyrimidine-5- carboxamide | (297) |
| 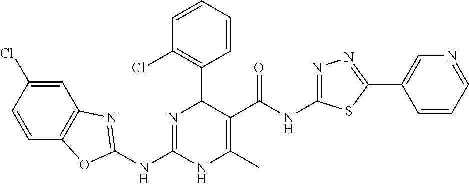 [SMILES: CC1=C(C(=O)Nc2nnc(-c3cccnc3)s2)C(c2ccccc2Cl)N=C(Nc2nc3cc(Cl)ccc3o2)N1] 4-((5-chlorobenzo[d]oxazol-2-yl)amino)-4-(2- chlorophenyl)-6-methyl-N-(5-(pyridin-3-yl)-1,3,4- thiadiazol-2-yl)-1,4-dihydropyrimidine-5- carboxamide | (298) |
| 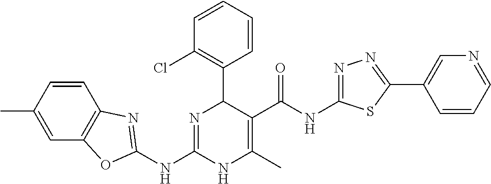 [SMILES: CC1=C(C(=O)Nc2nnc(-c3cccnc3)s2)C(c2ccccc2Cl)N=C(Nc2nc3ccc(C)cc3o2)N1] 4-(2-chlorophenyl)-6-methyl-2-((6- methylbenzo[d]oxazol-2-yl)amino)-N-(5-(pyridin-3- yl)-1,3,4-thiadiazol-2-yl)-1,4-dihydropyrimidine-5- carboxamide | (299) |
| 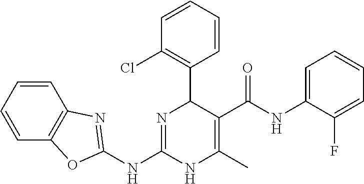 [SMILES: CC1=C(C(=O)Nc2ccccc2F)C(c2ccccc2Cl)N=C(Nc2nc3ccccc3o2)N1] 2-(benzo[d]oxazol-2-ylamino)-4-(2- chlorophenyl)-N-(2-fluorophenyl)-6-methyl-1,4- dihydropyrimidine-5-carboxamide | (300) |
| 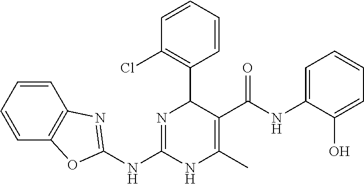 [SMILES: CC1=C(C(=O)Nc2ccccc2O)C(c2ccccc2Cl)N=C(Nc2nc3ccccc3o2)N1] 2-(benzo[d]oxazol-2-ylamino)-4-(2-chlorophenyl)- N-(2-hydroxyphenyl)-6-methyl-1,4- dihydropyrimidine-5-carboxamide | (301) |
| 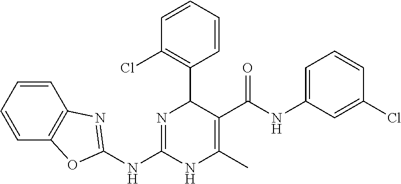 [SMILES: CC1=C(C(=O)Nc2cccc(Cl)c2)C(c2ccccc2Cl)N=C(Nc2nc3ccccc3o2)N1] 2-(benzo[d]oxazol-2-ylamino)-4-(2- chlorophenyl)-N-(3-chlorophenyl)-6-methyl-1,4- dihydropyrimidine-5-carboxamide | (302) |
| 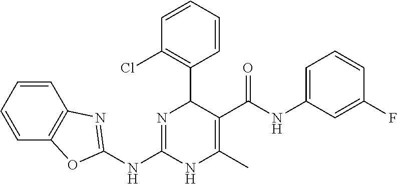 [SMILES: CC1=C(C(=O)Nc2cccc(F)c2)C(c2ccccc2Cl)N=C(Nc2nc3ccccc3o2)N1] 2-(benzo[d]oxazol-2-ylamino)-4-(2-chlorophenyl)- N-(3-fluorophenyl)-6-methyl-1,4-dihydropyrimidine- 5-carboxamide | (303) |
| 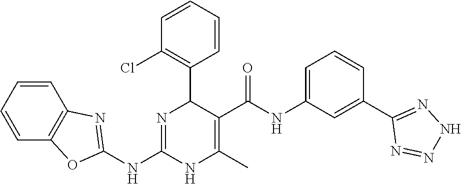 [SMILES: CC1=C(C(=O)Nc2cccc(-c3nn[nH]n3)c2)C(c2ccccc2Cl)N=C(Nc2nc3ccccc3o2)N1] N-(3-(2H-tetrazol-5-yl)phenyl)-2- (benzo[d]oxazol-2-ylamino)-4-(2-chlorophenyl)- 6-methyl-1,4-dihydropyrimidine-5-carboxamide | (304) |
| 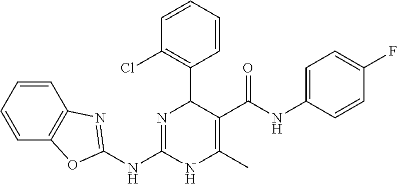 [SMILES: CC1=C(C(=O)Nc2ccc(F)cc2)C(c2ccccc2Cl)N=C(Nc2nc3ccccc3o2)N1] 2-(benzo[d]oxazol-2-ylamino)-4-(2-chlorophenyl)- N-(4-fluorophenyl)-6-methyl-1,4-dihydropyrimidine- 5-carboxamide | (305) |
| 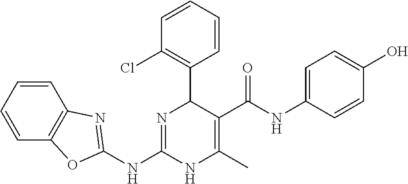 [SMILES: CC1=C(C(=O)Nc2ccc(O)cc2)C(c2ccccc2Cl)N=C(Nc2nc3ccccc3o2)N1] 2-(benzo[d]oxazol-2-ylamino)-4-(2- chlorophenyl)-N-(4-hydroxyphenyl)-6-methyl- 1,4-dihydropyrimidine-5-carboxamide | (306) |
| 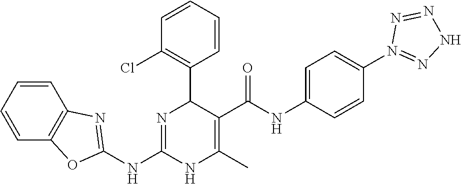 N-(4-(2H-tetrazol-5-yl)phenyl)-2-(benzo[d]oxazol-, 2-ylamino)-4-(2-chlorophenyl)-6-methyl-1,4- dihydropyrimidine-5-carboxamide | (307) |
| 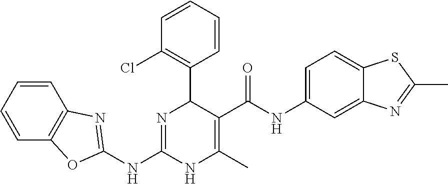 [SMILES: CC1=C(C(=O)Nc2ccc3sc(C)nc3c2)C(c2ccccc2Cl)N=C(Nc2nc3ccccc3o2)N1] 2-(benzo[d]oxazol-2-ylamino)-4-(2- chlorophenyl)-6-methyl-N-(2- methylbenzo[d]thiazol-5-yl)-1,4- dihydropyrimidine-5-carboxamide | (308) |
| 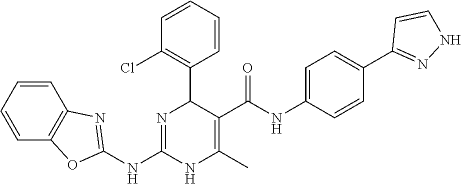 [SMILES: CC1=C(C(=O)Nc2ccc(-c3cc[nH]n3)cc2)C(c2ccccc2Cl)N=C(Nc2nc3ccccc3o2)N1] N-(4-(1H-pyrazol-3-yl)phenyl)-2-(benzo[d]oxazol-2- ylamino)-4-(2-chlorophenyl)-6-methyl-1,4- dihydropyrimidine-5-carboxamide | (309) |
| 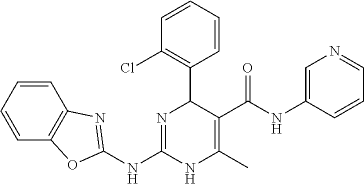 [SMILES: CC1=C(C(=O)Nc2cccnc2)C(c2ccccc2Cl)N=C(Nc2nc3ccccc3o2)N1] 2-(benzo[d]oxazol-2-ylamino)-4-(2- chlorophenyl)-6-methyl-N-(pyridin-3-yl)-1,4- dihydropyrimidine-5-carboxamide | (310) |
| 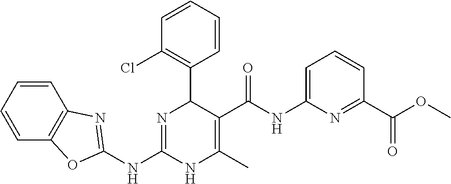 methyl 6-(2-(benzo[d]oxazol-2-ylamino)-4-(2- chlorophenyl)-6-methyl-1,4-dihydropyrimidine-5- carboxamido)picolinate | (311) |
| 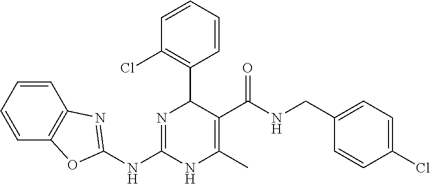 [SMILES: CC1=C(C(=O)NCc2ccc(Cl)cc2)C(c2ccccc2Cl)N=C(Nc2nc3ccccc3o2)N1] 2-(benzo[d]oxazol-2-ylamino)-N-(4- chlorobenzyl)-4-(2-chlorophenyl)-6-methyl-1,4- dihydropyrimidine-5-carboxamide | (312) |
| 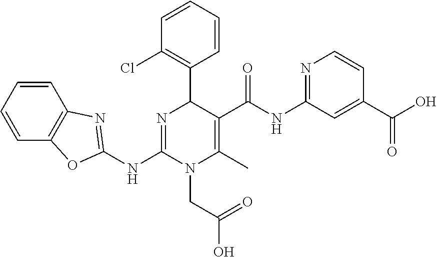 [SMILES: CC1=C(C(=O)Nc2cc(C(=O)O)ccn2)C(c2ccccc2Cl)N=C(Nc2nc3ccccc3o2)N1CC(=O)O] 2-(2-(benzo[d]oxazol-2-ylamino)-1- (carboxymethyl)-4-(2-chlorophenyl)-6-methyl-1,4- dihydropyrimidine-5-carboxamido)isonicotinic acid | (313) |
| 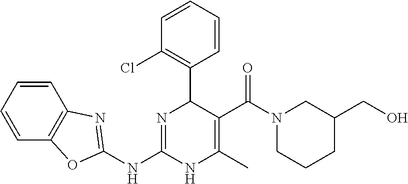 [SMILES: CC1=C(C(=O)N2CCCC(CO)C2)C(c2ccccc2Cl)N=C(Nc2nc3ccccc3o2)N1] (2-(benzo[d]oxazol-2-ylamino)-4-(2- chlorophenyl)-6-methyl-1,4-dihydropyrimidin-5- yl)(3-(hydroxymethyl)piperidin-1-yl)methanone | (314) |
| 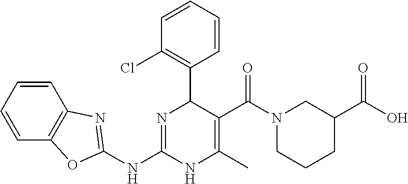 [SMILES: CC1=C(C(=O)N2CCCC(C(=O)O)C2)C(c2ccccc2Cl)N=C(Nc2nc3ccccc3o2)N1] 1-(2-(benzo[d]oxazol-2-ylamino)-4-(2- chlorophenyl)-6-methyl-1,4-dihydropyrimidine-5- carbonyl)piperidine-3-carboxylic acid | (315) |
| 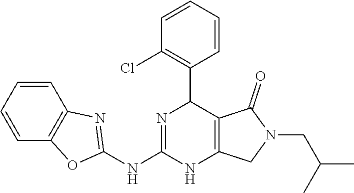 [SMILES: CC(C)CN1CC2=C(C1=O)C(c1ccccc1Cl)N=C(Nc1nc3ccccc3o1)N2] 2-(benzo[d]oxazol-2-ylamino)-4-(2- chlorophenyl)-6-isobutyl-1,4,6,7-tetrahydro-5H- pyrrolo[3,4-d]pyrimidin-5-one | (316) |
| 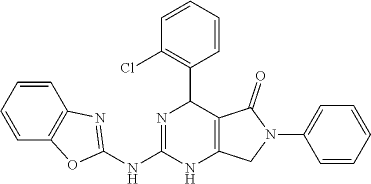 [SMILES: O=C1C2=C(CN1c1ccccc1)NC(Nc1nc3ccccc3o1)=NC2c1ccccc1Cl] 2-(benzo[d]oxazol-2-ylamino)-4-(2-chlorophenyl)- 6-phenyl-1,4,6,7-tetrahydro-5H-pyrrolo[3,4-d] pyrimidin-5-one | (317) |
| 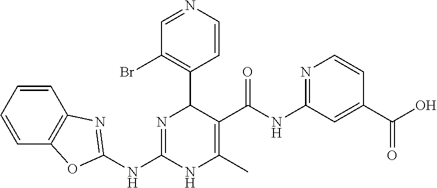 [SMILES: CC1=C(C(=O)Nc2cc(C(=O)O)ccn2)C(c2ccncc2Br)N=C(Nc2nc3ccccc3o2)N1] 2-(2-(benzo[d]oxazol-2-ylamino)-4-(3- bromopyridin-4-yl)-6-methyl-1,4- dihydropyrimidine-5-carboxamido)isonicotinic acid | (318) |
| 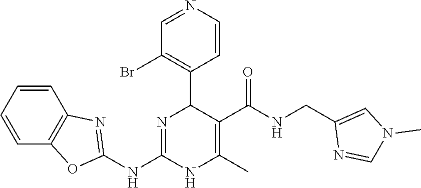 [SMILES: CC1=C(C(=O)NCc2cn(C)cn2)C(c2ccncc2Br)N=C(Nc2nc3ccccc3o2)N1] 2-(benzo[d]oxazol-2-ylamino)-4-(3-bromopyridin-4- yl)-6-methyl-N-((1-methyl-1H-imidazol-4- yl)methyl)-1,4-dihydropyrimidine-5-carboxamide | (319) |
| 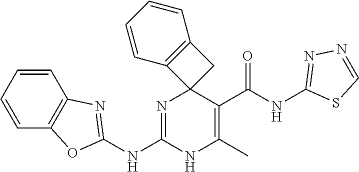 [SMILES: CC1=C(C(=O)Nc2nncs2)C2(Cc3ccccc32)N=C(Nc2nc3ccccc3o2)N1] 2′-(benzo[d]oxazol-2-ylamino)-6′-methyl-N- (1,3,4-thiadiazol-2-yl)-1′H- spiro[bicyclo[4.2.0]octane-7,4′-pyrimidine]- 1(6),2,4-triene-5′-carboxamide | (320) |
| 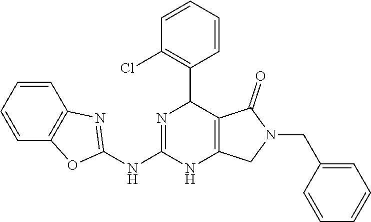 [SMILES: O=C1C2=C(CN1Cc1ccccc1)NC(Nc1nc3ccccc3o1)=NC2c1ccccc1Cl] 2-(benzo[d]oxazol-2-ylamino)-6-benzyl-4-(2- chlorophenyl)-1,4,6,7-tetrahydro-5H-pyrrolo[3,4- d]pyrimidin-5-one | (321) |
| 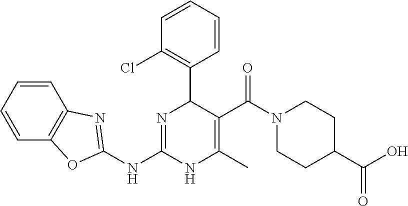 [SMILES: CC1=C(C(=O)N2CCC(C(=O)O)CC2)C(c2ccccc2Cl)N=C(Nc2nc3ccccc3o2)N1] 1-(2-(benzo[d]oxazol-2-ylamino)-4-(2- chlorophenyl)-6-methyl-1,4-dihydropyrimidine-5- carbonyl)piperidine-4-carboxylic acid | (322) |
| 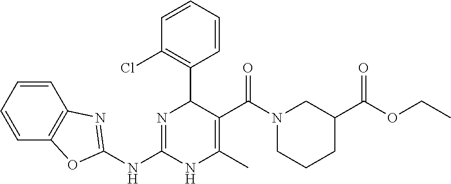 [SMILES: CCOC(=O)C1CCCN(C(=O)C2=C(C)NC(Nc3nc4ccccc4o3)=NC2c2ccccc2Cl)C1] ethyl 1-(2-(benzo[d]oxazol-2-ylamino)-4-(2- chlorophenyl)-6-methyl-1,4-dihydropyrimidine-5- carbonyl)piperidine-3-carboxylate | (323) |
| 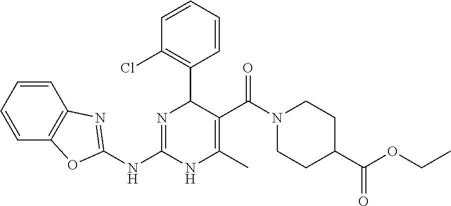 [SMILES: CCOC(=O)C1CCN(C(=O)C2=C(C)NC(Nc3nc4ccccc4o3)=NC2c2ccccc2Cl)CC1] ethyl 1-(2-(benzo[d]oxazol-2-ylamino)-4-(2- chlorophenyl)-6-methyl-1,4-dihydropyrimidine-5- carbonyl)piperidine-4-carboxylate | (324) |
| 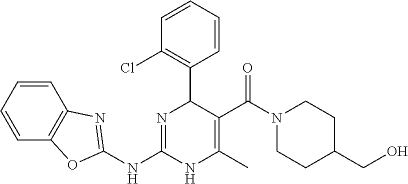 [SMILES: CC1=C(C(=O)N2CCC(CO)CC2)C(c2ccccc2Cl)N=C(Nc2nc3ccccc3o2)N1] (2-(benzo[d]oxazol-2-ylamino)-4-(2-chlorophenyl)- 6-methyl-1,4-dihydropyrimidin-5-yl)(4- (hydroxymethyl)piperidin-l -yl)methanone | (325) |

### Example 13

### GALK Lead Profile

### Compound 126

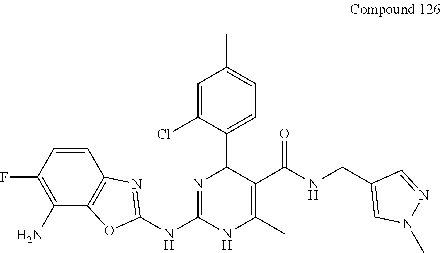 [SMILES: CC1=C(C(=O)NCc2cnn(C)c2)C(c2ccc(C)cc2Cl)N=C(Nc2nc3ccc(F)c(N)c3o2)N1]

### 2-((7-amino-6-fluorobenzo[d]oxazol-2-yl)amino)-4-(2-chloro-4-methylphenyl)-6-methyl-N-((1-methyl-1H-pyrazol-4-yl)methyl)-1,4-dihydropyrimidine-5-carboxamide

Chemical Formula: C₂₅H₂₄ClFN₈O₂

### Molecular Weight: 522.97

tPSA: 129

### CLogP: 4.32

hGALK IC₅₀ (mM) 0.048
mGALK IC₅₀ (mM) 0.69
Solubility 21, 1.3 mg/mL ˜40, 2.5 mM
PAMPA 10⁻⁶ cm/s: 390, 485

### RLM, MLM, HLM t, min: 16, 13, 47

In vivo Pharmacokinetic and Pharmacodynamic Experiments

For all pharmacokinetic and pharmacodynamic studies, female CD1 mice were housed in isolated cages and provided food and water ad libitum.

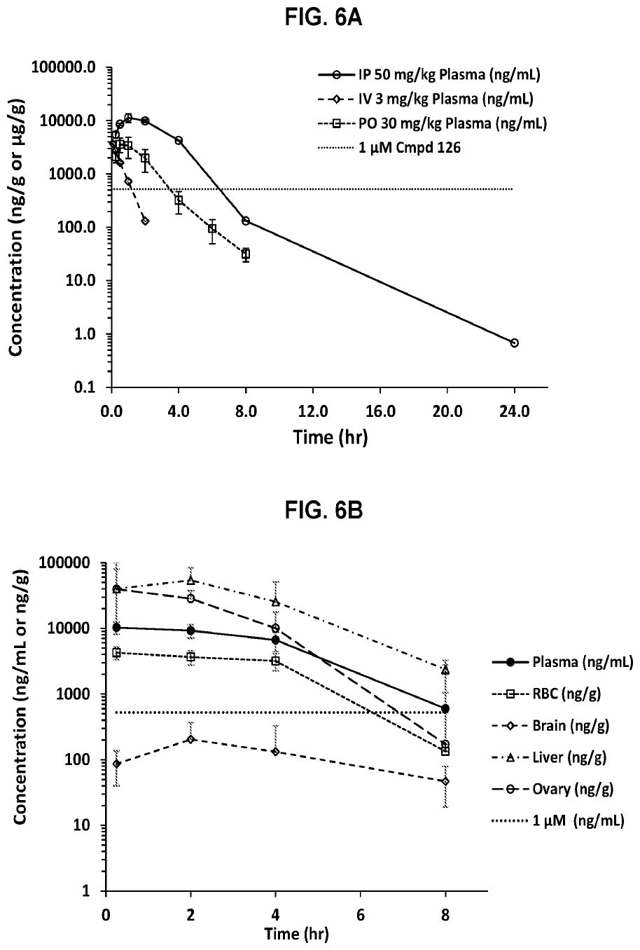

Compound 126 was administered was administered intraperitoneally (50 mg/kg), intravenously (3 mg/kg), and orally (30 mg/kg) (n=3 for each route). Blood samples were obtained at +15 min, +30 min, +1 h, +2 h, +4 h, +6 h, +8 h, +24 h. The blood was processed to obtain serum/plasma and analyzed for the presence of Compound 126 using HPLC/LC mass spectrometry using a standard curve. The data for each of the three mice for each route of administration were averaged and standard deviations were obtained. Results are shown in FIG. 6A-B.

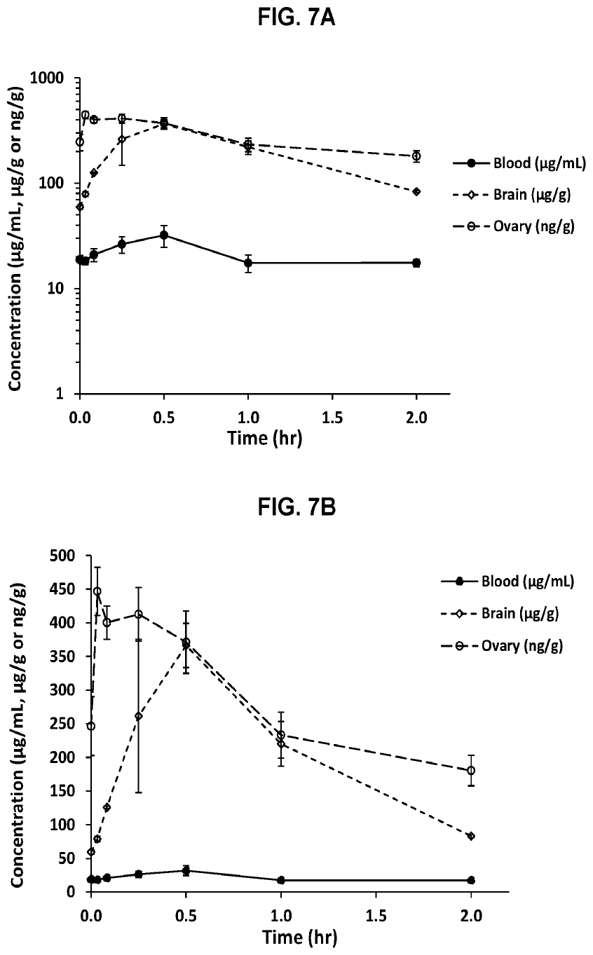

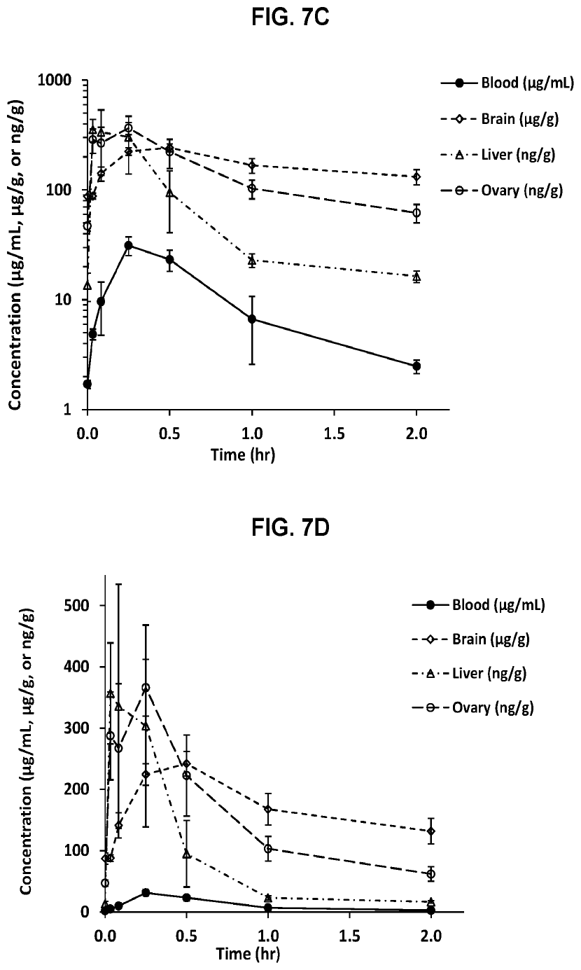

A single IP dose of galactose (578 mg/kg) or ¹³C₆-galactose (572 mg/kg) was administered to different cohorts of female CD1 mice (n=5). Animals were euthanized at +2 min, +5 min, +15 min, +30 min, +1 hr, +2 hr. Blood and organs (brain, liver, ovary) were harvested; galactose-1 phosphate or ¹³C₆-galactose-1 phosphate contents were analyzed by LC/Mass Spectrometry. The tissue data for each of the five mice were averaged and standard deviations were obtained. Results are shown in Table 22 and FIG. 7A-D.

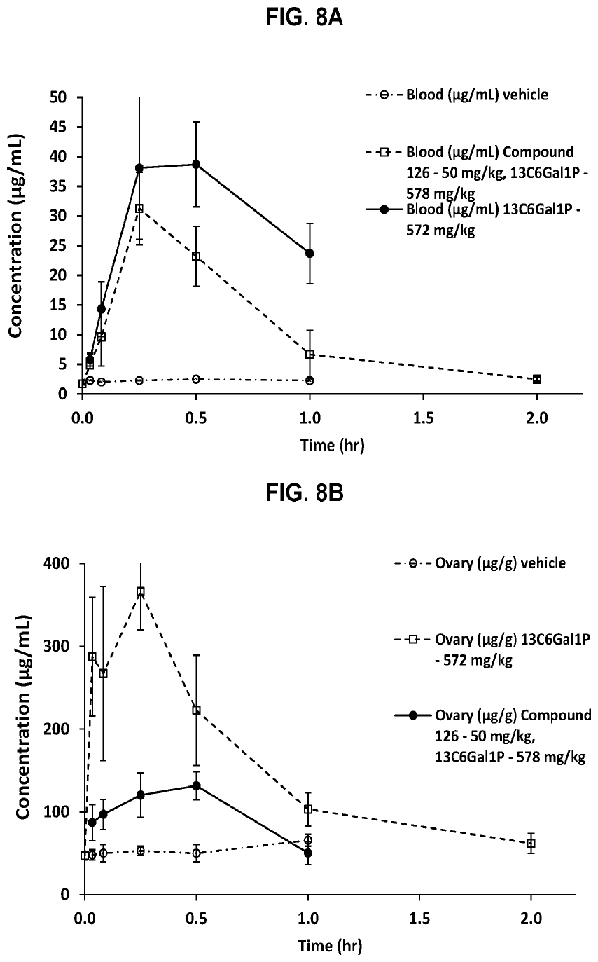

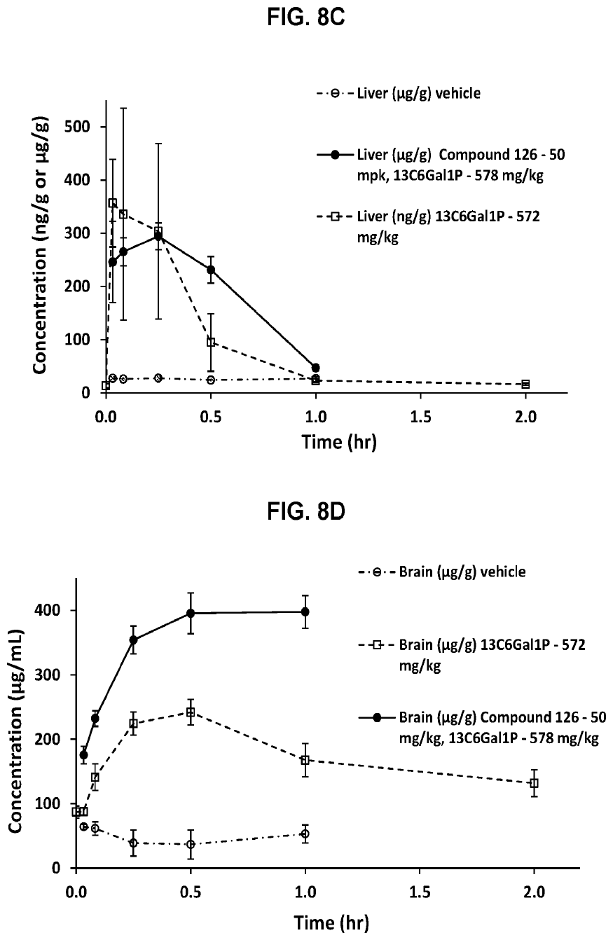

A single IP dose Compound 126 (50 mg/kg)/vehicle was administered to each animal. One hour later, a single galactose (578 mg/kg) or ¹³C₆-galactose (572 mg/kg) was administered to different cohorts of female CD1 mice (n=5). Animals were euthanized at +2 min, +5 min, +15 min, +30 min, +1 hr, +2 hr. Blood and organs (brain, liver, ovary) were harvested; galactose-1 phosphate or ¹³C₆-galactose-1 phosphate contents were analyzed by LC Mass Spectrometry. The tissue data for each of the five mice were averaged and standard deviations were obtained. Results are shown in Table 22 and FIG. 8A-D.

TABLE 22
Female CD1 Mice (n = 5): Dose 50 mg/kg Compound 126, then after 1 h
dose 400 mg/kg ¹³C₆ Galactose; Monitor ¹³C₆-Galactose-1-Phosphate

| Time | | Study 1 | Study 2 |
|---|---|---|---|
| 0 | — | Vehicle 1 | 50 mg/kg IP |
| | | (Compound 126) IP | Compound 126 |
| 1 h | 578 mg/kg IP | Vehicle 2 (¹³C₆ | 578 mg/kg IP (400) ¹³C₆ |
| | Galactose (400) | galactose) IP | galactose |
| 1 h 2 min | n = 5 | n = 5 | n = 5 |
| 1 h 5 min | n = 5 | n = 5 | n = 5 |
| 1 h 15 min | n = 5 | n = 5 | n = 5 |
| 1 h 30 min | n = 5 | n = 5 | n = 5 |
| 2 h | n = 5 | n = 5 | n = 5 |

Vehicle 1: 5% NMP, 25% PEG 300, 70% 15% solution of solutol HS 15 in water
Vehicle 2: DI water

What is claimed:

1. A composition for inhibiting a galactokinase activity comprising Formula I or a salt thereof: 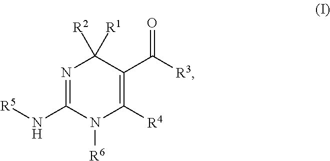
wherein:
R¹ and R² are each independently selected from hydrogen, C₆-C₁₂-aryl, C₁-C₆-alkyl, or C₅-C₁₂-heteroaryl, with the proviso that at least one of R¹ or R² is not hydrogen; or where R¹ and R² taken together, including the atoms to which they are attached, form a 5- to 7-membered carbocycle or a 5- to 7-membered heterocycle;
R³ is selected from —NH—C₁-C₆-alkyl-C₅-C₁₂-heteroaryl, —NH—C₁-C₆-alkyl-C₆-C₁₂-aryl, —NH—C₁-C₆-alkyl-NH₂, —NH—C₁-C₆-alkyl-NH(C₁₋₄-alkyl), —NH—C₁-C₆-alkyl-N(C₁₋₄-alkyl)₂, or —NR⁷R⁸ wherein R⁷ and R⁸ are each independently selected from hydrogen, C₁-C₆-alkyl, C₃-C₆-cycloalkyl, C₃-C₆-heterocycloalkyl, C₆-C₁₂-aryl, or C₅-C₁₂-heteroaryl; or wherein R⁷ and R⁸ together, including the atoms to which they are attached, form a 5- to 6-membered heteroaryl or a 4- to 6-membered heterocycloalkyl ring;
R⁴ is selected from C₁-C₆-alkyl, hydrogen, C₁-C₂-alkoxy-C₁-C₂-alkyl, C₆-C₁₂-aryl, or C₅-C₁₂-heteroaryl; or
R⁵ is selected from:
substituted benzoxazolyl or substituted benzothioxazolyl, wherein R⁵ is substituted with two, one, three, or four substituents independently selected from —NH₂, halogen, C₁-C₆-alkyl, C₆-C₁₂-aryl, —CF₃, C₁-C₄-alkoxy, C₆-C₁₂-aryloxy, —NH(C₁-C₄-alkyl), —CO₂H, or —N(C₁-C₄-alkyl)₂, with the proviso that R⁵ cannot be substituted with more than one —NH₂ group; or
unsubstituted oxazolopyridinyl, unsubstituted tetrahydrobenzo[d]oxazolyl, or unsubstituted 2-phenyloxazolyl; and
R⁶ is selected from hydrogen, C₅-C₁₂-heteroaryl, or C₁-C₆-alkyl.

2. The composition of claim 1, wherein:
R¹ is hydrogen;
R² is phenyl or pyridinyl, wherein R² is unsubstituted or substituted with one, two, or three substituents independently selected from halogen, C₁-C₄-alkyl, —CF₃, C₁-C₂-alkoxy, C₆-C₁₂-aryl, C₄-C₆-heterocyclyl, C₅-C₁₂-heteroaryl, —CONH₂, —NH₂, —CN, —CO₂H, or —SO₂NH₂; and
R⁵ is selected from:
substituted benzoxazolyl or substituted benzothioxazolyl, wherein R⁵ is substituted with one or more substituents independently selected from C₁-C₆-alkyl, halogen, —CF₃, C₁-C₄-alkoxy, —NH₂, or —CO₂H; or
unsubstituted oxazolopyridinyl.

3. The composition of claim 1, wherein R⁵ is selected from 4-fluoro-benzoxazol-2-yl, 5-fluoro-benzoxazol-2-yl, 6-fluoro-benzoxazol-2-yl, 7-fluoro-benzoxazol-2-yl, 4-chloro-benzoxazol-2-yl, 5-chloro-benzoxazol-2-yl, 6-chloro-benzoxazol-2-yl, 7-chloro-benzoxazol-2-yl, 5-bromo-benzoxazol-2-yl, 4-methyl-benzoxazol-2-yl, 5-methyl-benzoxazol-2-yl, 6-methyl-benzoxazol-2-yl, 7-methyl-benzoxazol-2-yl, 5-methoxyl-benzoxazol-2-yl, 6-methoxyl-benzoxazol-2-yl, 7-methoxyl-benzoxazol-2-yl, 4-amino-benzoxazol-2-yl, 5-amino-benzoxazol-2-yl, 7-amino-benzoxazol-2-yl, 7-trifluoromethyl-benzoxazol-2-yl, 7-trifluoromethoxyl-benzoxazol-2-yl, 7-nitro-benzoxazol-2-yl, 7-amino-6-fluoro-benzoxazol-2-yl, or 7-carboxylic acid-6-fluoro-benzoxazol-2-yl.

4. The composition of claim 1, wherein R⁵ is selected from 6-fluoro-benzoxazol-2-yl, 5-methyl-benzoxazol-2-yl, 7-methyl benzoxazol-2-yl, or 7-amino-benzoxazol-2-yl, or 7-amino-6-fluoro-benzoxazol-2-yl.

5. The composition of claim 1, wherein R⁵ is 7-amino-6-fluoro-benzoxazol-2-yl.

6. The composition of claim 1, wherein the compound is selected from:

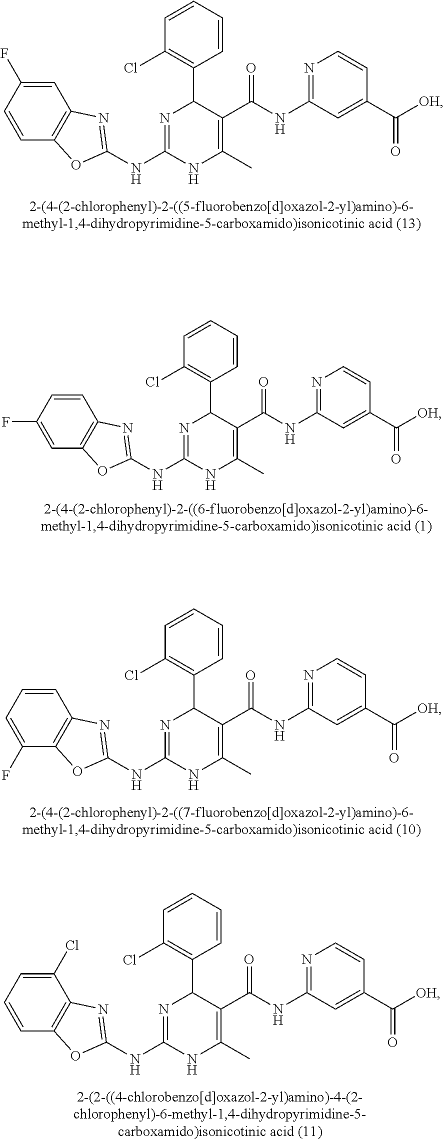 [SMILES: CC1=C(C(=O)Nc2cc(C(=O)O)ccn2)C(c2ccccc2Cl)N=C(Nc2nc3cc(F)ccc3o2)N1 ; CC1=C(C(=O)Nc2cc(C(=O)O)ccn2)C(c2ccccc2Cl)N=C(Nc2nc3ccc(F)cc3o2)N1 ; CC1=C(C(=O)Nc2cc(C(=O)O)ccn2)C(c2ccccc2Cl)N=C(Nc2nc3cccc(F)c3o2)N1 ; CC1=C(C(=O)Nc2cc(C(=O)O)ccn2)C(c2ccccc2Cl)N=C(Nc2nc3c(Cl)cccc3o2)N1]

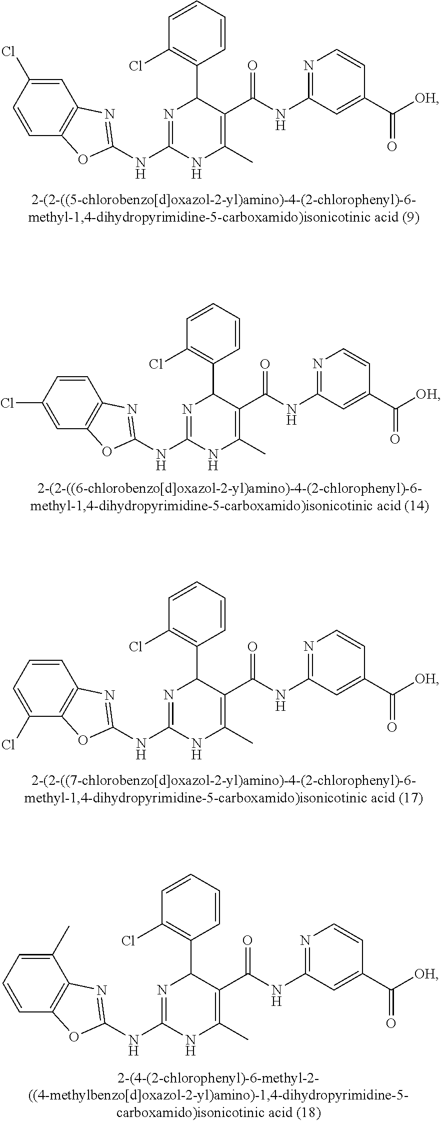 [SMILES: CC1=C(C(=O)Nc2cc(C(=O)O)ccn2)C(c2ccccc2Cl)N=C(Nc2nc3cc(Cl)ccc3o2)N1 ; CC1=C(C(=O)Nc2cc(C(=O)O)ccn2)C(c2ccccc2Cl)N=C(Nc2nc3ccc(Cl)cc3o2)N1 ; CC1=C(C(=O)Nc2cc(C(=O)O)ccn2)C(c2ccccc2Cl)N=C(Nc2nc3cccc(Cl)c3o2)N1 ; CC1=C(C(=O)Nc2cc(C(=O)O)ccn2)C(c2ccccc2Cl)N=C(Nc2nc3c(C)cccc3o2)N1]

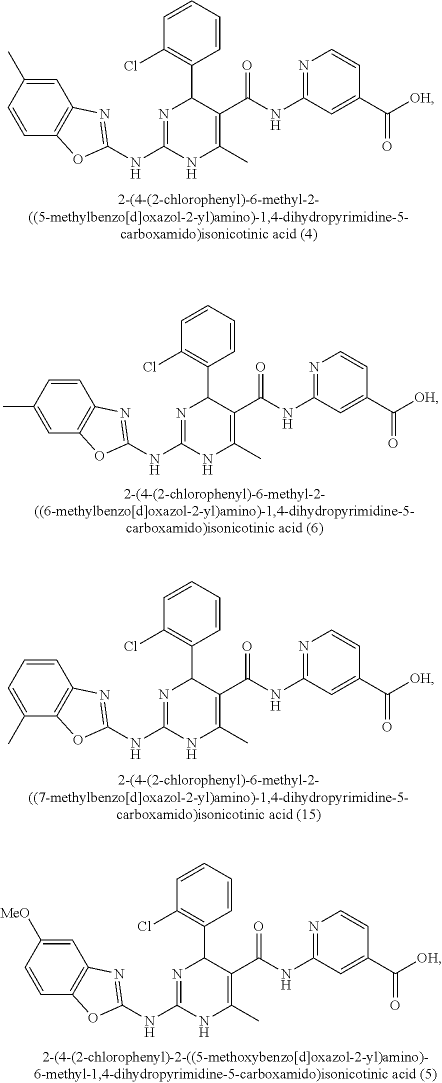 [SMILES: CC1=C(C(=O)Nc2cc(C(=O)O)ccn2)C(c2ccccc2Cl)N=C(Nc2nc3cc(C)ccc3o2)N1 ; CC1=C(C(=O)Nc2cc(C(=O)O)ccn2)C(c2ccccc2Cl)N=C(Nc2nc3ccc(C)cc3o2)N1 ; CC1=C(C(=O)Nc2cc(C(=O)O)ccn2)C(c2ccccc2Cl)N=C(Nc2nc3cccc(C)c3o2)N1 ; COc1ccc2oc(NC3=NC(c4ccccc4Cl)C(C(=O)Nc4cc(C(=O)O)ccn4)=C(C)N3)nc2c1]

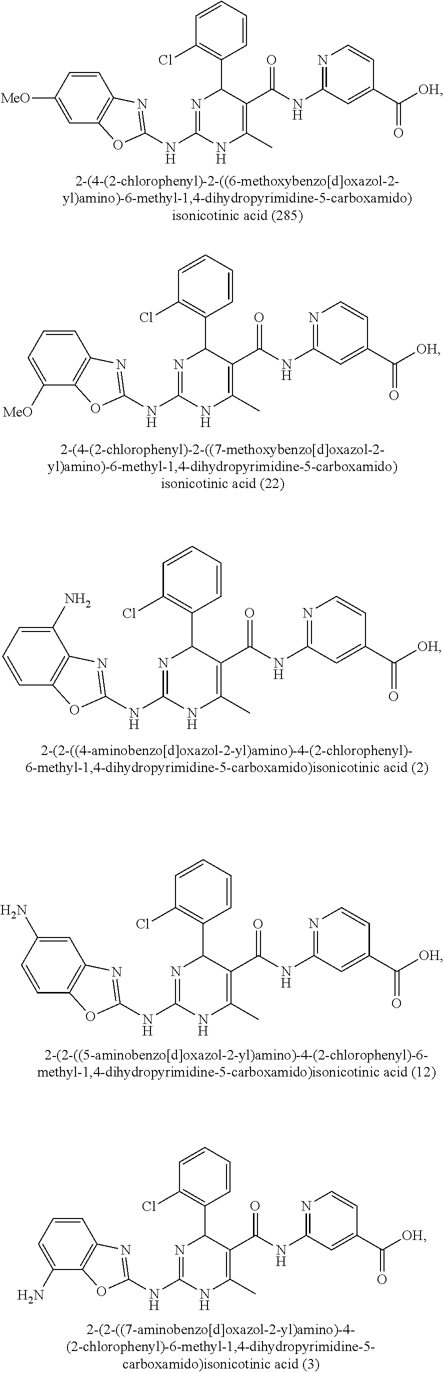 [SMILES: COc1ccc2nc(NC3=NC(c4ccccc4Cl)C(C(=O)Nc4cc(C(=O)O)ccn4)=C(C)N3)oc2c1 ; COc1cccc2nc(NC3=NC(c4ccccc4Cl)C(C(=O)Nc4cc(C(=O)O)ccn4)=C(C)N3)oc12 ; CC1=C(C(=O)Nc2cc(C(=O)O)ccn2)C(c2ccccc2Cl)N=C(Nc2nc3c(N)cccc3o2)N1 ; CC1=C(C(=O)Nc2cc(C(=O)O)ccn2)C(c2ccccc2Cl)N=C(Nc2nc3cc(N)ccc3o2)N1 ; CC1=C(C(=O)Nc2cc(C(=O)O)ccn2)C(c2ccccc2Cl)N=C(Nc2nc3cccc(N)c3o2)N1]

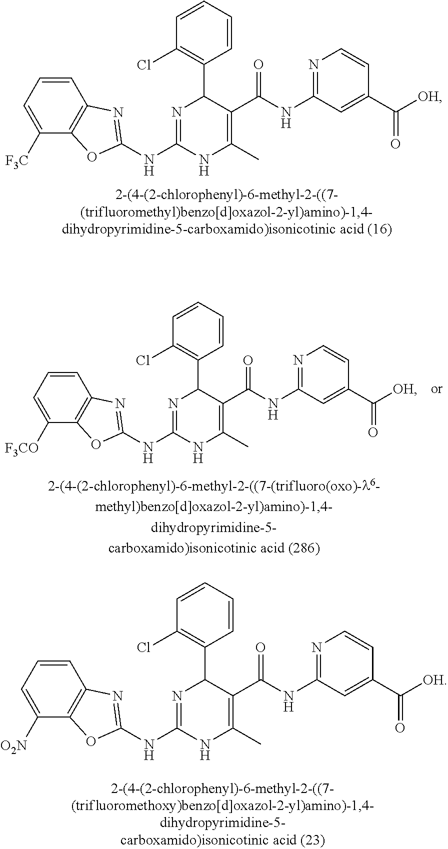 [SMILES: CC1=C(C(=O)Nc2cc(C(=O)O)ccn2)C(c2ccccc2Cl)N=C(Nc2nc3cccc(C(F)(F)F)c3o2)N1 ; CC1=C(C(=O)Nc2cc(C(=O)O)ccn2)C(c2ccccc2Cl)N=C(Nc2nc3cccc(OC(F)(F)F)c3o2)N1 ; CC1=C(C(=O)Nc2cc(C(=O)O)ccn2)C(c2ccccc2Cl)N=C(Nc2nc3cccc([N+](=O)[O-])c3o2)N1]

7. The composition of claim 1, wherein the compound is selected from:

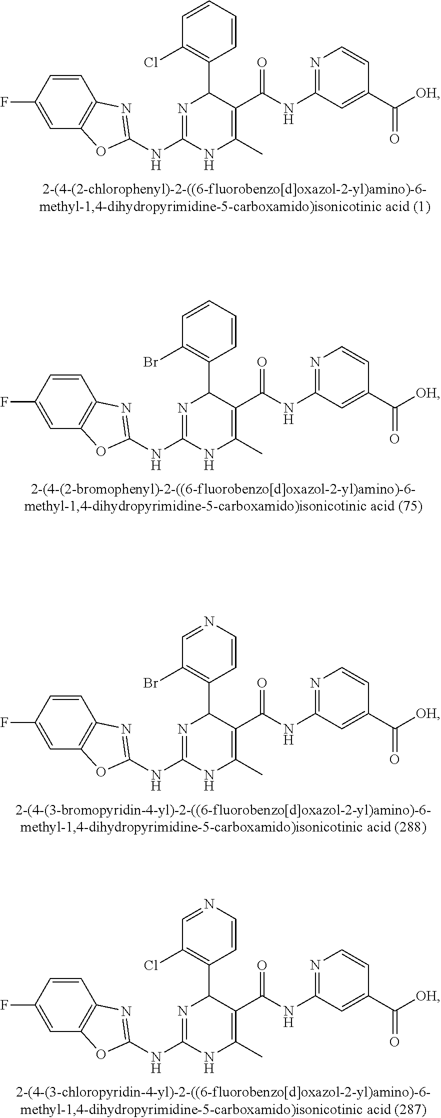 [SMILES: CC1=C(C(=O)Nc2cc(C(=O)O)ccn2)C(c2ccccc2Cl)N=C(Nc2nc3ccc(F)cc3o2)N1 ; CC1=C(C(=O)Nc2cc(C(=O)O)ccn2)C(c2ccccc2Br)N=C(Nc2nc3ccc(F)cc3o2)N1 ; CC1=C(C(=O)Nc2cc(C(=O)O)ccn2)C(c2ccncc2Br)N=C(Nc2nc3ccc(F)cc3o2)N1 ; CC1=C(C(=O)Nc2cc(C(=O)O)ccn2)C(c2ccncc2Cl)N=C(Nc2nc3ccc(F)cc3o2)N1]

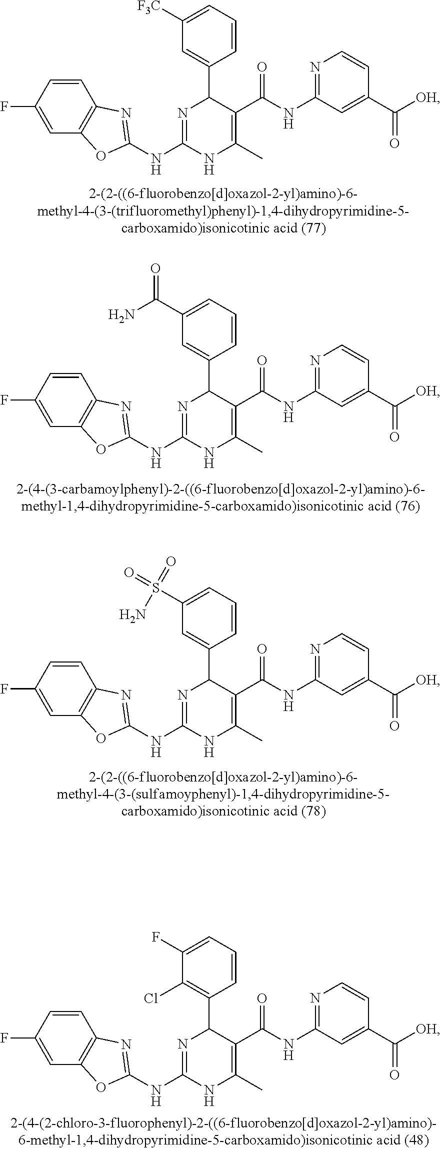 [SMILES: CC1=C(C(=O)Nc2cc(C(=O)O)ccn2)C(c2cccc(C(F)(F)F)c2)N=C(Nc2nc3ccc(F)cc3o2)N1 ; CC1=C(C(=O)Nc2cc(C(=O)O)ccn2)C(c2cccc(C(N)=O)c2)N=C(Nc2nc3ccc(F)cc3o2)N1 ; CC1=C(C(=O)Nc2cc(C(=O)O)ccn2)C(c2cccc(S(N)(=O)=O)c2)N=C(Nc2nc3ccc(F)cc3o2)N1 ; CC1=C(C(=O)Nc2cc(C(=O)O)ccn2)C(c2cccc(F)c2Cl)N=C(Nc2nc3ccc(F)cc3o2)N1]

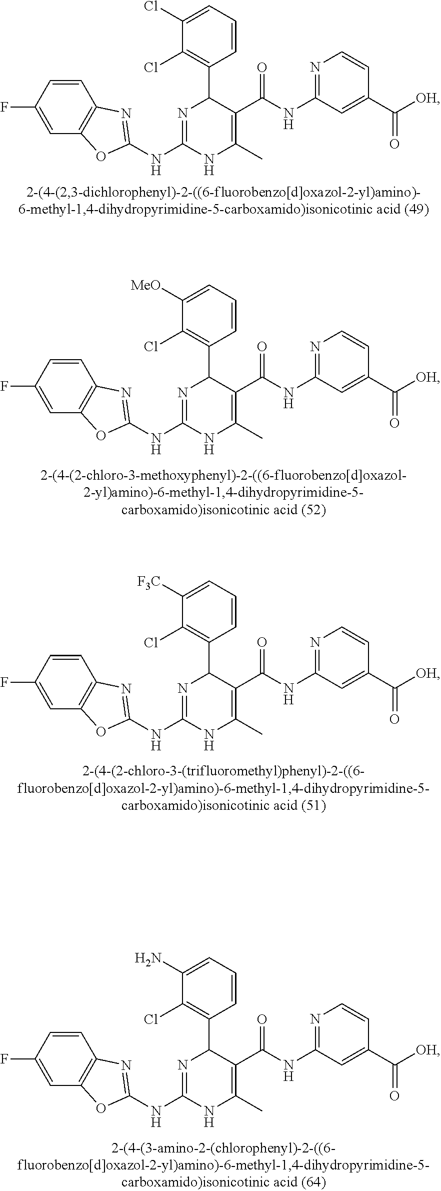 [SMILES: CC1=C(C(=O)Nc2cc(C(=O)O)ccn2)C(c2cccc(Cl)c2Cl)N=C(Nc2nc3ccc(F)cc3o2)N1 ; COc1cccc(C2N=C(Nc3nc4ccc(F)cc4o3)NC(C)=C2C(=O)Nc2cc(C(=O)O)ccn2)c1Cl ; CC1=C(C(=O)Nc2cc(C(=O)O)ccn2)C(c2cccc(C(F)(F)F)c2Cl)N=C(Nc2nc3ccc(F)cc3o2)N1 ; CC1=C(C(=O)Nc2cc(C(=O)O)ccn2)C(c2cccc(N)c2Cl)N=C(Nc2nc3ccc(F)cc3o2)N1]

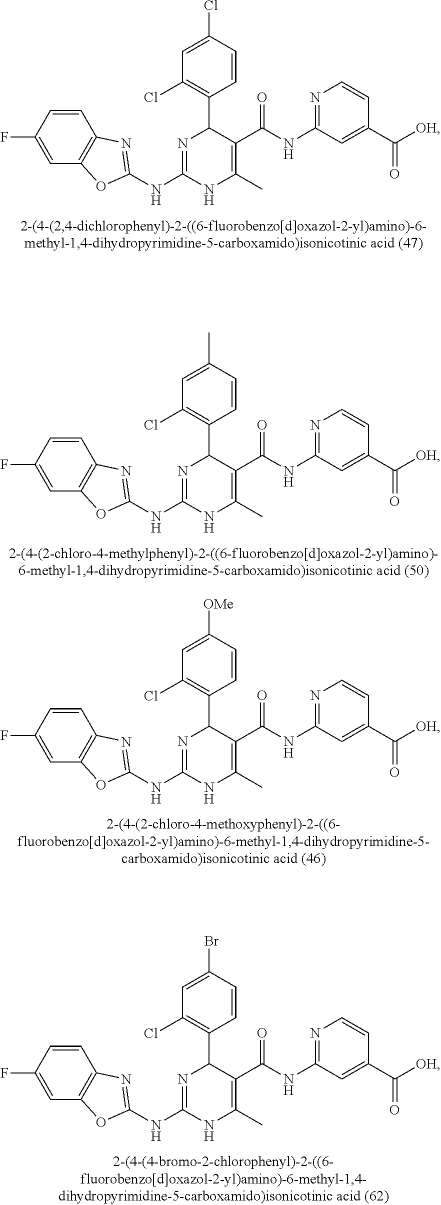 [SMILES: CC1=C(C(=O)Nc2cc(C(=O)O)ccn2)C(c2ccc(Cl)cc2Cl)N=C(Nc2nc3ccc(F)cc3o2)N1 ; CC1=C(C(=O)Nc2cc(C(=O)O)ccn2)C(c2ccc(C)cc2Cl)N=C(Nc2nc3ccc(F)cc3o2)N1 ; COc1ccc(C2N=C(Nc3nc4ccc(F)cc4o3)NC(C)=C2C(=O)Nc2cc(C(=O)O)ccn2)c(Cl)c1 ; CC1=C(C(=O)Nc2cc(C(=O)O)ccn2)C(c2ccc(Br)cc2Cl)N=C(Nc2nc3ccc(F)cc3o2)N1]

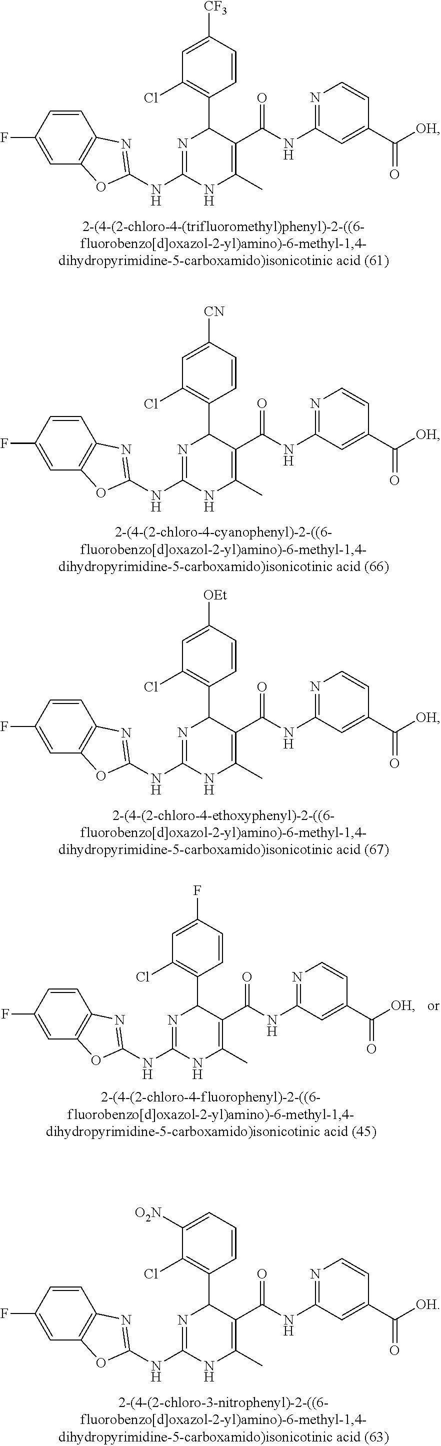 [SMILES: CC1=C(C(=O)Nc2cc(C(=O)O)ccn2)C(c2ccc(C(F)(F)F)cc2Cl)N=C(Nc2nc3ccc(F)cc3o2)N1 ; CC1=C(C(=O)Nc2cc(C(=O)O)ccn2)C(c2ccc(C#N)cc2Cl)N=C(Nc2nc3ccc(F)cc3o2)N1 ; CCOc1ccc(C2N=C(Nc3nc4ccc(F)cc4o3)NC(C)=C2C(=O)Nc2cc(C(=O)O)ccn2)c(Cl)c1 ; CC1=C(C(=O)Nc2cc(C(=O)O)ccn2)C(c2ccc(F)cc2Cl)N=C(Nc2nc3ccc(F)cc3o2)N1 ; CC1=C(C(=O)Nc2cc(C(=O)O)ccn2)C(c2cccc([N+](=O)[O-])c2Cl)N=C(Nc2nc3ccc(F)cc3o2)N1]

8. The composition of claim 1, wherein the compound is selected from:

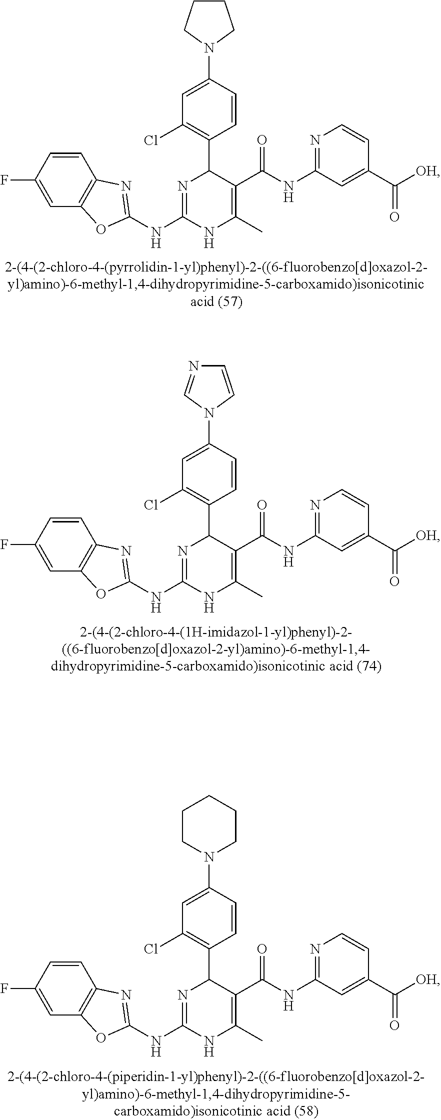 [SMILES: CC1=C(C(=O)Nc2cc(C(=O)O)ccn2)C(c2ccc(N3CCCC3)cc2Cl)N=C(Nc2nc3ccc(F)cc3o2)N1 ; CC1=C(C(=O)Nc2cc(C(=O)O)ccn2)C(c2ccc(-n3ccnc3)cc2Cl)N=C(Nc2nc3ccc(F)cc3o2)N1 ; CC1=C(C(=O)Nc2cc(C(=O)O)ccn2)C(c2ccc(N3CCCCC3)cc2Cl)N=C(Nc2nc3ccc(F)cc3o2)N1]

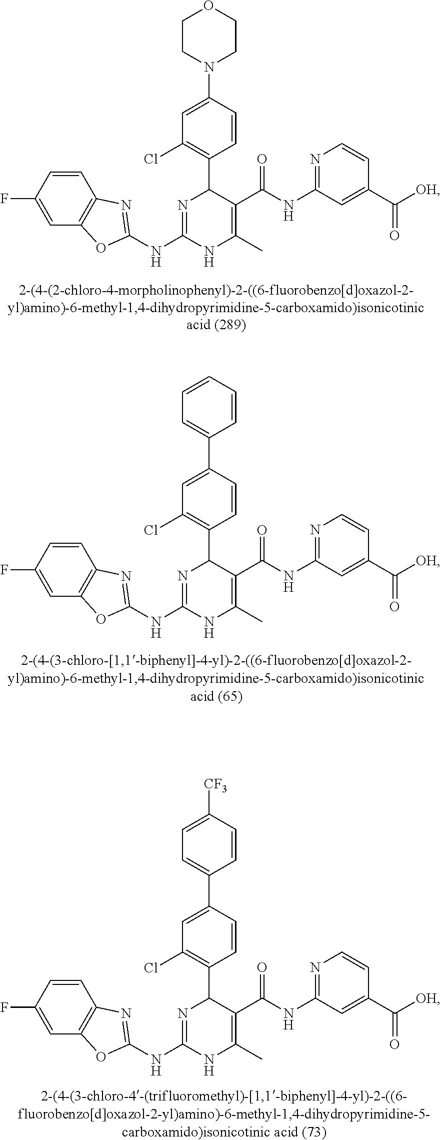 [SMILES: CC1=C(C(=O)Nc2cc(C(=O)O)ccn2)C(c2ccc(N3CCOCC3)cc2Cl)N=C(Nc2nc3ccc(F)cc3o2)N1 ; CC1=C(C(=O)Nc2cc(C(=O)O)ccn2)C(c2ccc(-c3ccccc3)cc2Cl)N=C(Nc2nc3ccc(F)cc3o2)N1 ; CC1=C(C(=O)Nc2cc(C(=O)O)ccn2)C(c2ccc(-c3ccc(C(F)(F)F)cc3)cc2Cl)N=C(Nc2nc3ccc(F)cc3o2)N1]

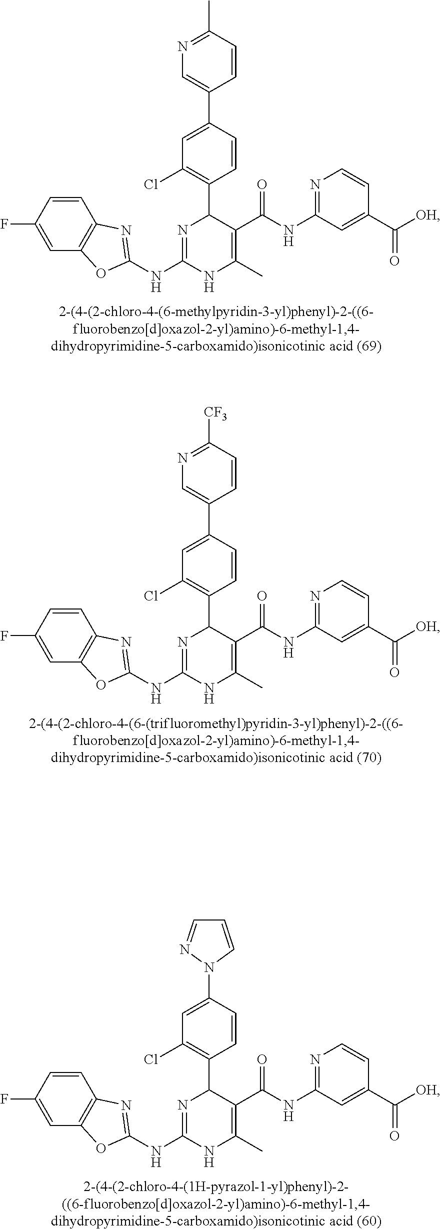 [SMILES: CC1=C(C(=O)Nc2cc(C(=O)O)ccn2)C(c2ccc(-c3ccc(C)nc3)cc2Cl)N=C(Nc2nc3ccc(F)cc3o2)N1 ; CC1=C(C(=O)Nc2cc(C(=O)O)ccn2)C(c2ccc(-c3ccc(C(F)(F)F)nc3)cc2Cl)N=C(Nc2nc3ccc(F)cc3o2)N1 ; CC1=C(C(=O)Nc2cc(C(=O)O)ccn2)C(c2ccc(-n3cccn3)cc2Cl)N=C(Nc2nc3ccc(F)cc3o2)N1]

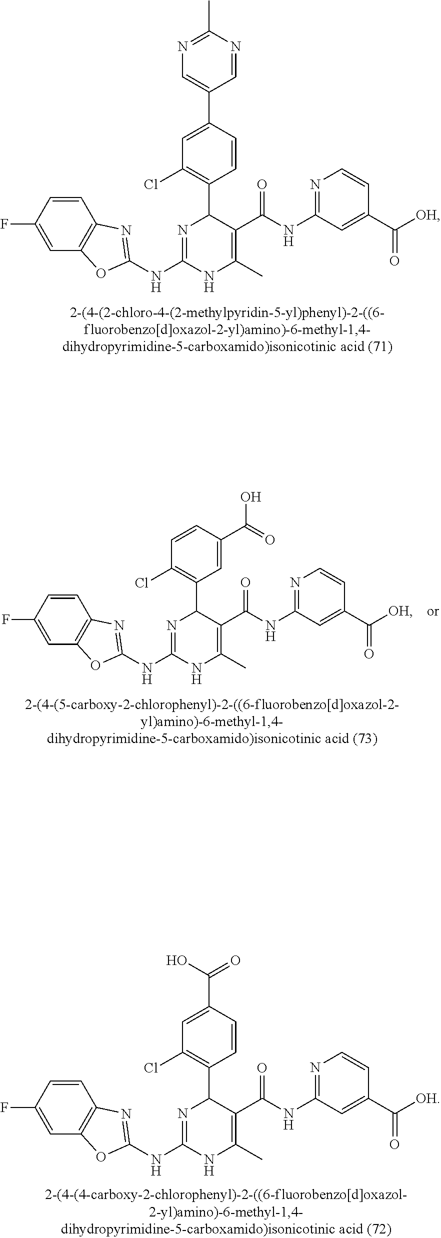 [SMILES: CC1=C(C(=O)Nc2cc(C(=O)O)ccn2)C(c2ccc(-c3cnc(C)nc3)cc2Cl)N=C(Nc2nc3ccc(F)cc3o2)N1 ; CC1=C(C(=O)Nc2cc(C(=O)O)ccn2)C(c2cc(C(=O)O)ccc2Cl)N=C(Nc2nc3ccc(F)cc3o2)N1 ; CC1=C(C(=O)Nc2cc(C(=O)O)ccn2)C(c2ccc(C(=O)O)cc2Cl)N=C(Nc2nc3ccc(F)cc3o2)N1]

9. The composition of claim 1, wherein the compound is selected from:

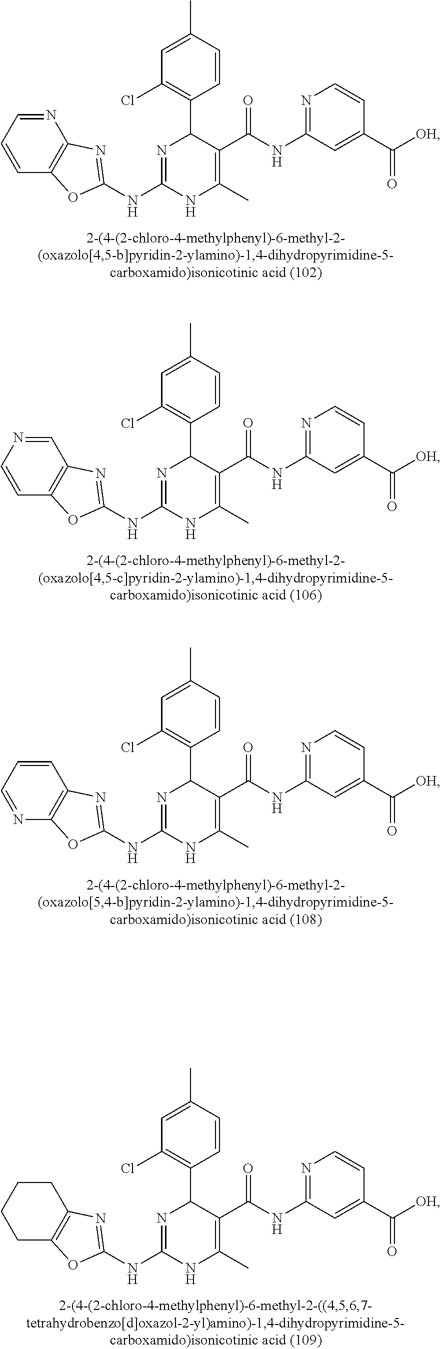 [SMILES: CC1=C(C(=O)Nc2cc(C(=O)O)ccn2)C(c2ccc(C)cc2Cl)N=C(Nc2nc3ncccc3o2)N1 ; CC1=C(C(=O)Nc2cc(C(=O)O)ccn2)C(c2ccc(C)cc2Cl)N=C(Nc2nc3cnccc3o2)N1 ; CC1=C(C(=O)Nc2cc(C(=O)O)ccn2)C(c2ccc(C)cc2Cl)N=C(Nc2nc3cccnc3o2)N1 ; CC1=C(C(=O)Nc2cc(C(=O)O)ccn2)C(c2ccc(C)cc2Cl)N=C(Nc2nc3c(o2)CCCC3)N1]

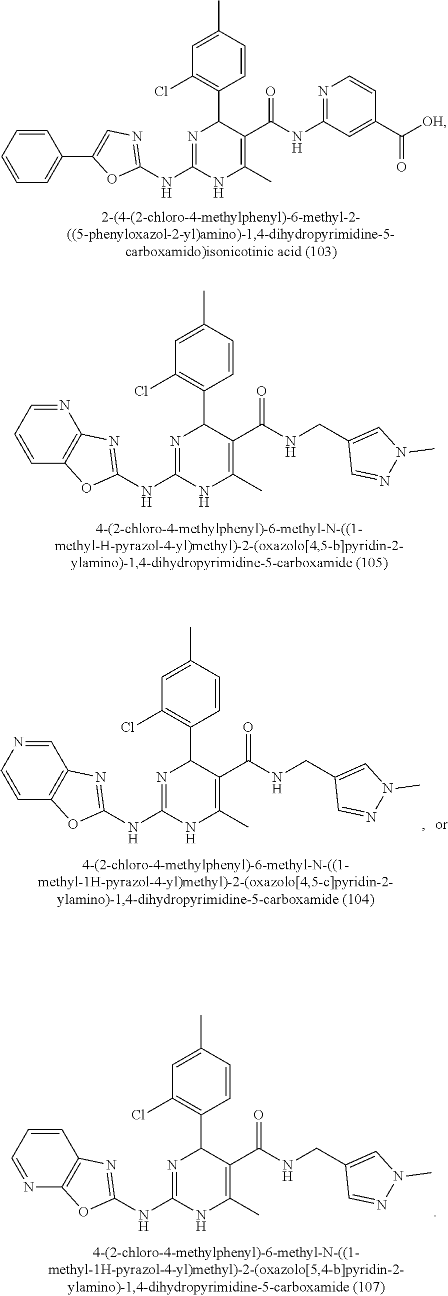 [SMILES: CC1=C(C(=O)Nc2cc(C(=O)O)ccn2)C(c2ccc(C)cc2Cl)N=C(Nc2ncc(-c3ccccc3)o2)N1 ; CC1=C(C(=O)NCc2cnn(C)c2)C(c2ccc(C)cc2Cl)N=C(Nc2nc3ncccc3o2)N1 ; CC1=C(C(=O)NCc2cnn(C)c2)C(c2ccc(C)cc2Cl)N=C(Nc2nc3cnccc3o2)N1 ; CC1=C(C(=O)NCc2cnn(C)c2)C(c2ccc(C)cc2Cl)N=C(Nc2nc3cccnc3o2)N1]

10. The composition of claim 1, wherein the compound is selected from:

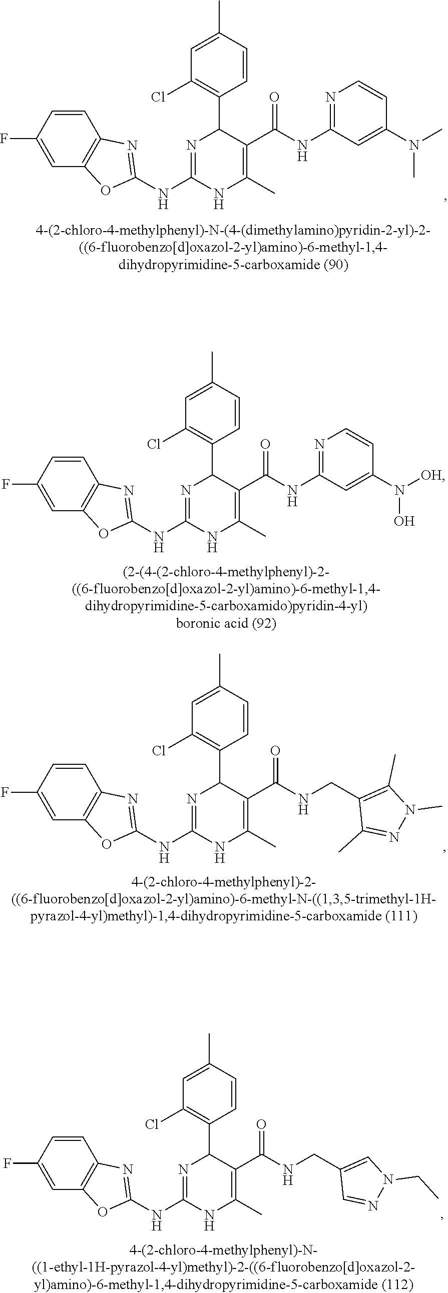 [SMILES: CC1=C(C(=O)Nc2cc(N(C)C)ccn2)C(c2ccc(C)cc2Cl)N=C(Nc2nc3ccc(F)cc3o2)N1 ; CC1=C(C(=O)Nc2cc(N(O)O)ccn2)C(c2ccc(C)cc2Cl)N=C(Nc2nc3ccc(F)cc3o2)N1 ; CC1=C(C(=O)NCc2c(C)nn(C)c2C)C(c2ccc(C)cc2Cl)N=C(Nc2nc3ccc(F)cc3o2)N1 ; CCn1cc(CNC(=O)C2=C(C)NC(Nc3nc4ccc(F)cc4o3)=NC2c2ccc(C)cc2Cl)cn1]

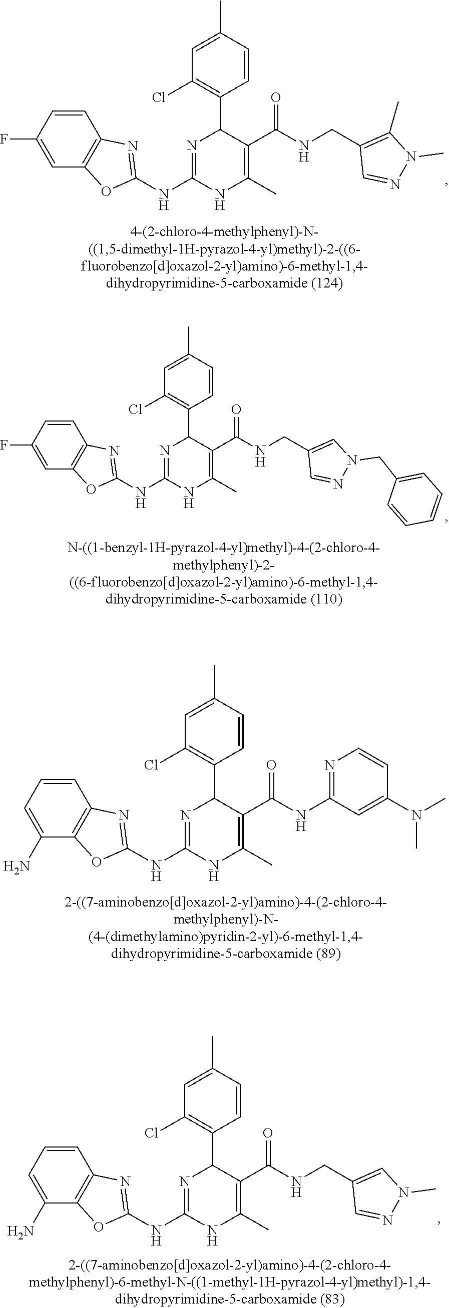 [SMILES: CC1=C(C(=O)NCc2cnn(C)c2C)C(c2ccc(C)cc2Cl)N=C(Nc2nc3ccc(F)cc3o2)N1 ; CC1=C(C(=O)NCc2cnn(Cc3ccccc3)c2)C(c2ccc(C)cc2Cl)N=C(Nc2nc3ccc(F)cc3o2)N1 ; CC1=C(C(=O)Nc2cc(N(C)C)ccn2)C(c2ccc(C)cc2Cl)N=C(Nc2nc3cccc(N)c3o2)N1 ; CC1=C(C(=O)NCc2cnn(C)c2)C(c2ccc(C)cc2Cl)N=C(Nc2nc3cccc(N)c3o2)N1]

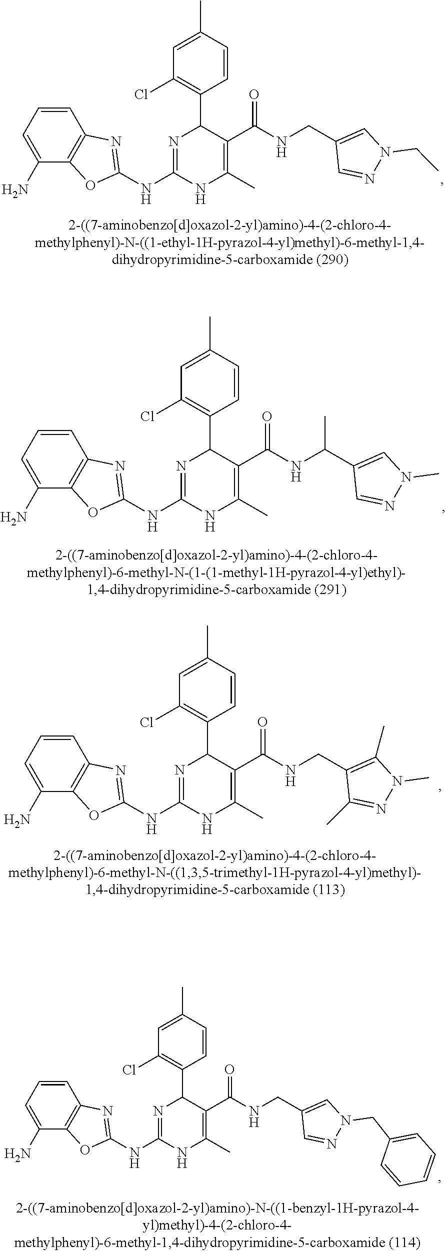 [SMILES: CCn1cc(CNC(=O)C2=C(C)NC(Nc3nc4cccc(N)c4o3)=NC2c2ccc(C)cc2Cl)cn1 ; CC1=C(C(=O)NC(C)c2cnn(C)c2)C(c2ccc(C)cc2Cl)N=C(Nc2nc3cccc(N)c3o2)N1 ; CC1=C(C(=O)NCc2c(C)nn(C)c2C)C(c2ccc(C)cc2Cl)N=C(Nc2nc3cccc(N)c3o2)N1 ; CC1=C(C(=O)NCc2cnn(Cc3ccccc3)c2)C(c2ccc(C)cc2Cl)N=C(Nc2nc3cccc(N)c3o2)N1]

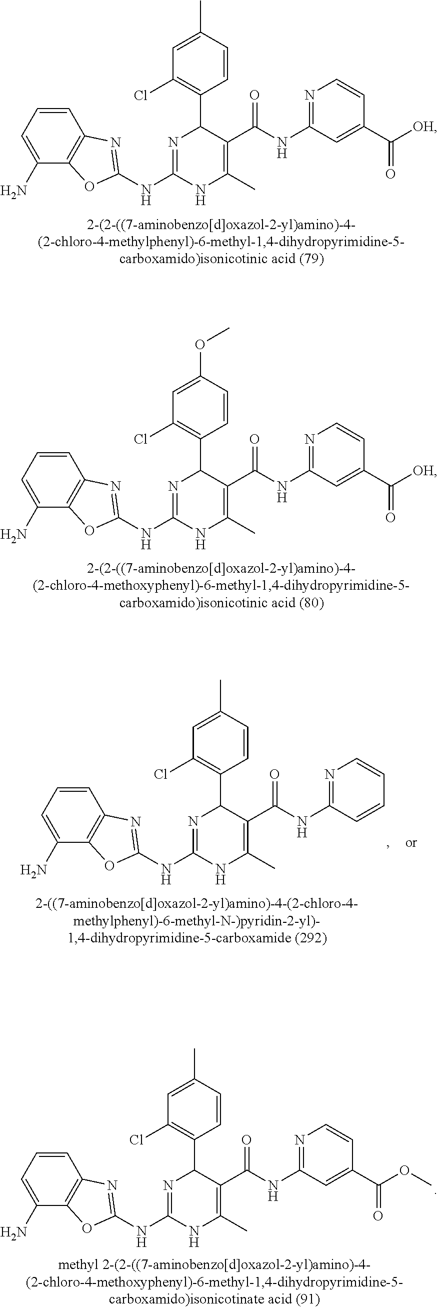 [SMILES: CC1=C(C(=O)Nc2cc(C(=O)O)ccn2)C(c2ccc(C)cc2Cl)N=C(Nc2nc3cccc(N)c3o2)N1 ; COc1ccc(C2N=C(Nc3nc4cccc(N)c4o3)NC(C)=C2C(=O)Nc2cc(C(=O)O)ccn2)c(Cl)c1 ; CC1=C(C(=O)Nc2ccccn2)C(c2ccc(C)cc2Cl)N=C(Nc2nc3cccc(N)c3o2)N1 ; COC(=O)c1ccnc(NC(=O)C2=C(C)NC(Nc3nc4cccc(N)c4o3)=NC2c2ccc(C)cc2Cl)c1]

11. The composition of claim 1, wherein the compound is selected from:

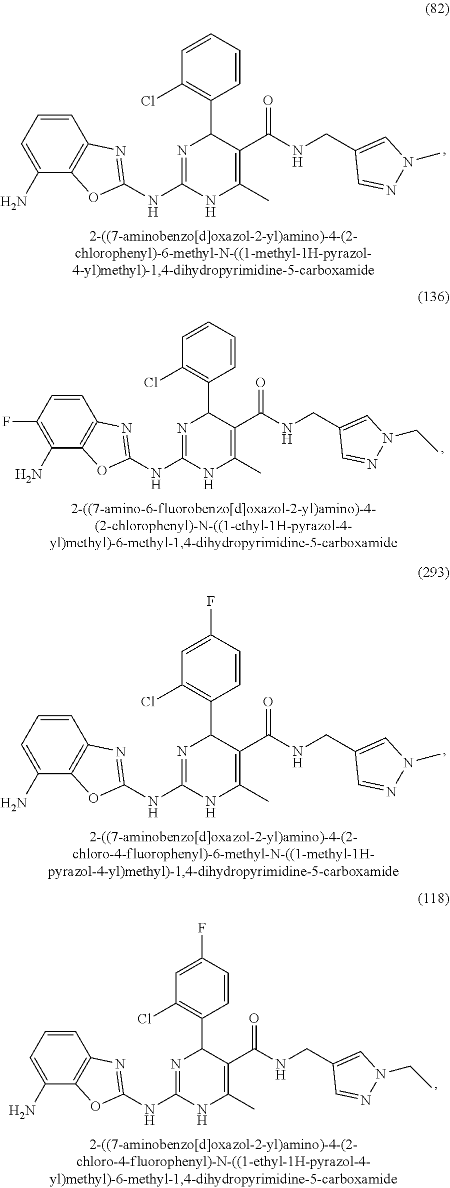

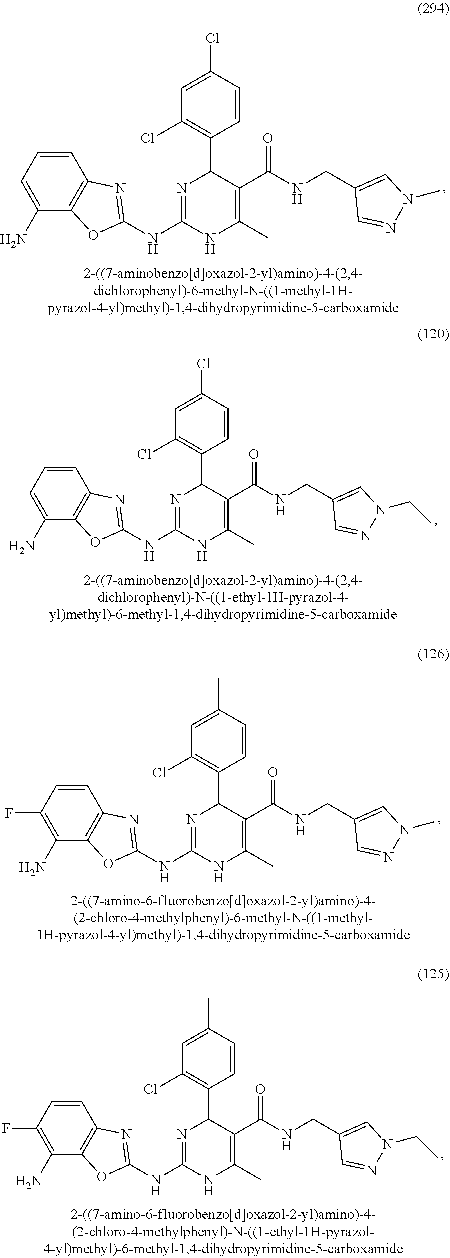

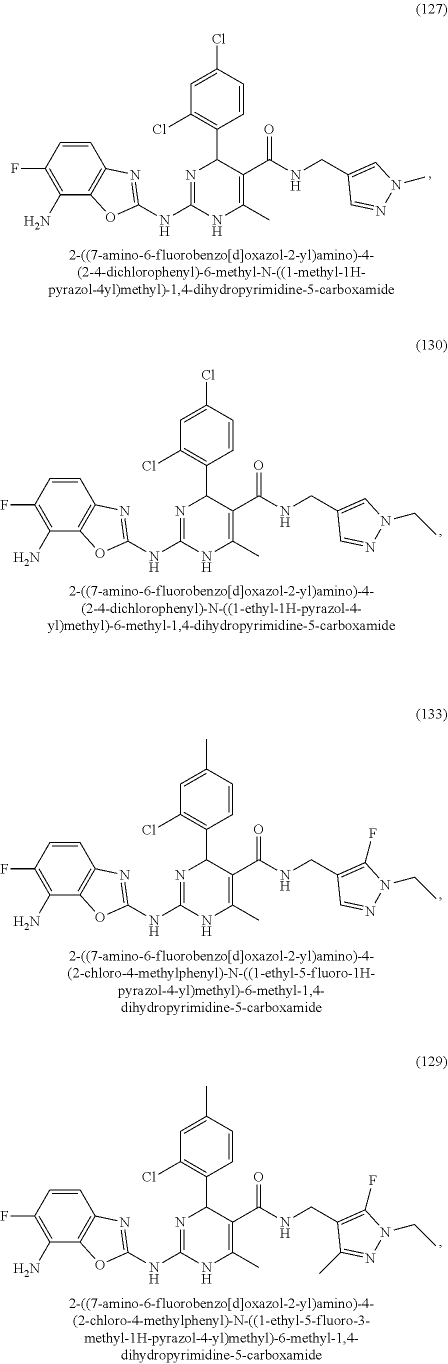

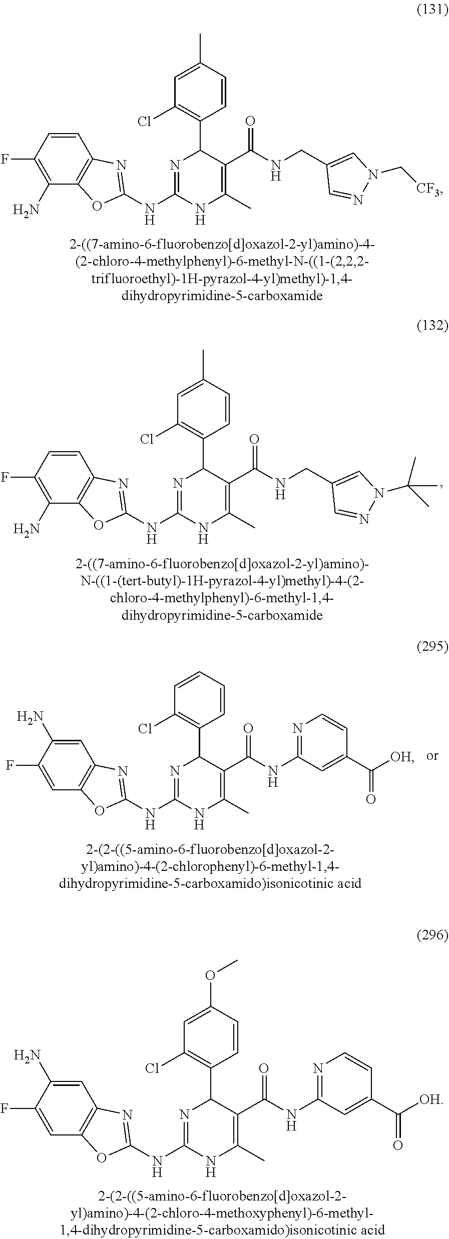 [SMILES: CC1=C(C(=O)NCc2cnn(CC(F)(F)F)c2)C(c2ccc(C)cc2Cl)N=C(Nc2nc3ccc(F)c(N)c3o2)N1 ; CC1=C(C(=O)NCc2cnn(C(C)(C)C)c2)C(c2ccc(C)cc2Cl)N=C(Nc2nc3ccc(F)c(N)c3o2)N1 ; "," ; CC1=C(C(=O)Nc2cc(C(=O)O)ccn2)C(c2ccccc2Cl)N=C(Nc2nc3cc(N)c(F)cc3o2)N1 ; "or" ; COc1ccc(C2N=C(Nc3nc4cc(N)c(F)cc4o3)NC(C)=C2C(=O)Nc2cc(C(=O)O)ccn2)c(Cl)c1]

12. The composition of claim 1, wherein the compound is selected from:

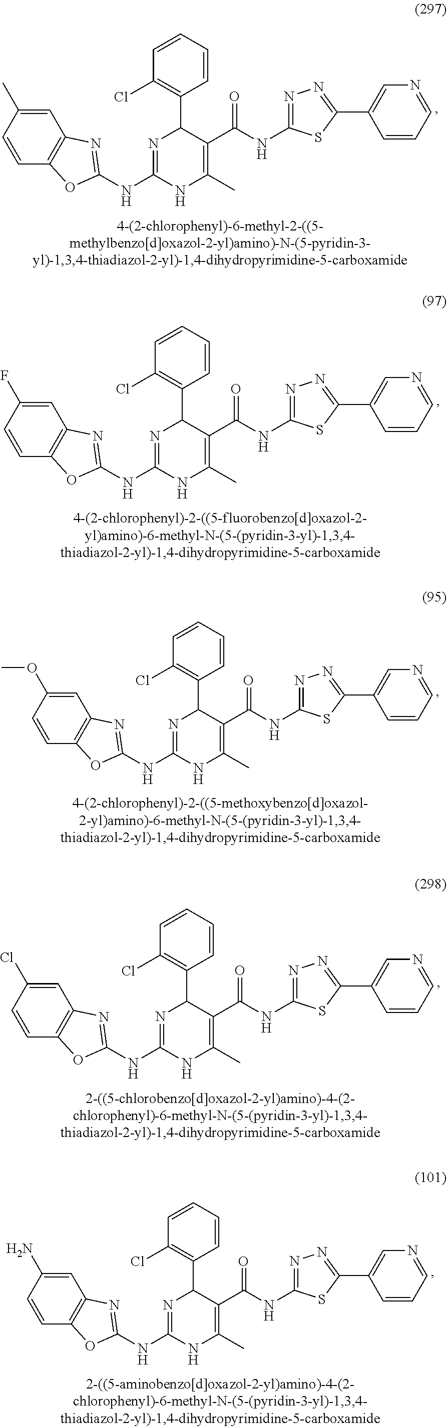

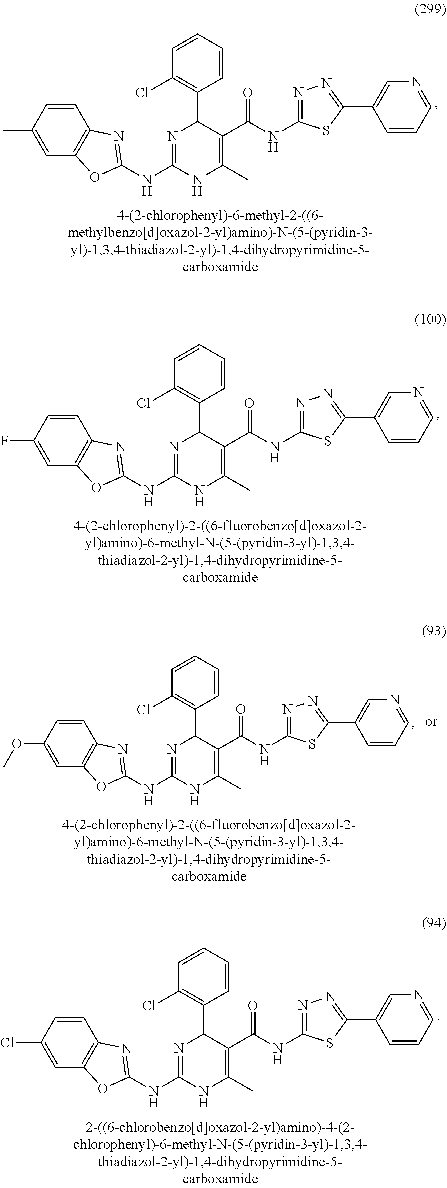

13. The composition of claim 1, wherein the compound is selected from:

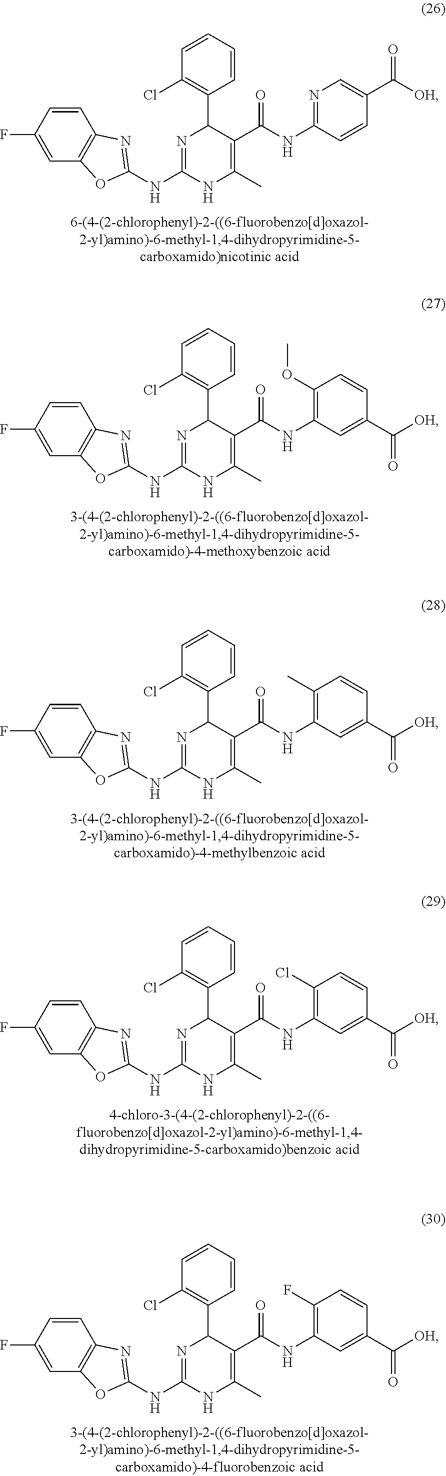 [SMILES: CC1=C(C(=O)Nc2ccc(C(=O)O)cn2)C(c2ccccc2Cl)N=C(Nc2nc3ccc(F)cc3o2)N1 ; COc1ccc(C(=O)O)cc1NC(=O)C1=C(C)NC(Nc2nc3ccc(F)cc3o2)=NC1c1ccccc1Cl ; CC1=C(C(=O)Nc2cc(C(=O)O)ccc2C)C(c2ccccc2Cl)N=C(Nc2nc3ccc(F)cc3o2)N1 ; CC1=C(C(=O)Nc2cc(C(=O)O)ccc2Cl)C(c2ccccc2Cl)N=C(Nc2nc3ccc(F)cc3o2)N1 ; CC1=C(C(=O)Nc2cc(C(=O)O)ccc2F)C(c2ccccc2Cl)N=C(Nc2nc3ccc(F)cc3o2)N1]

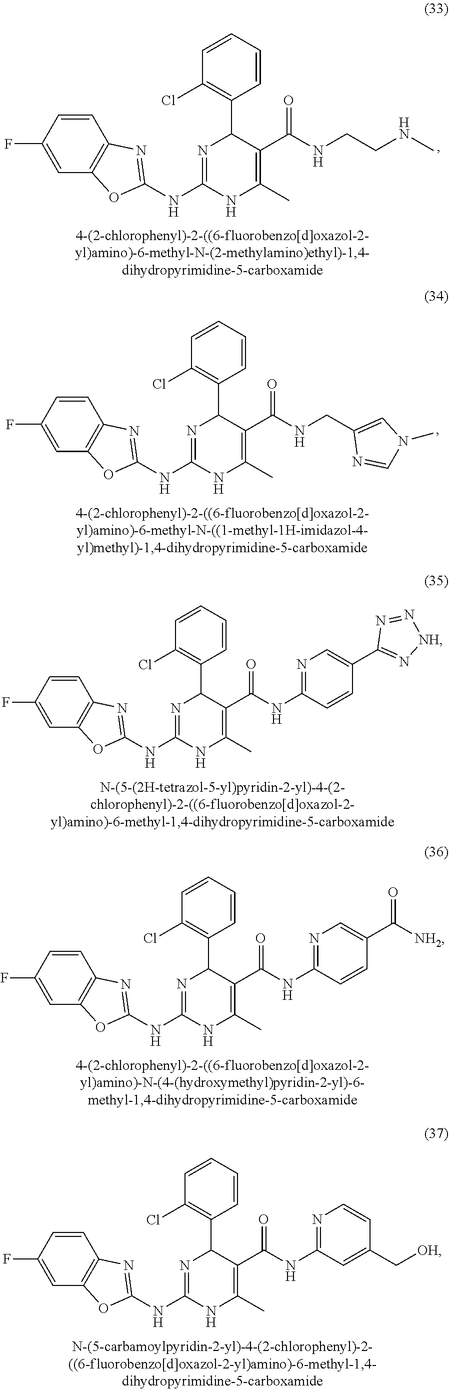

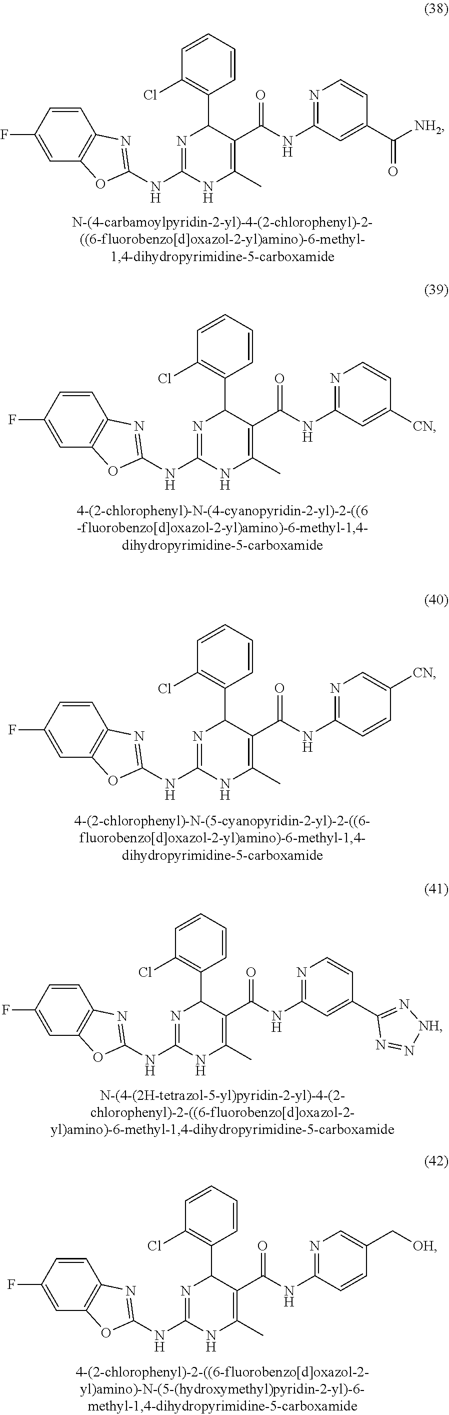 [SMILES: CC1=C(C(=O)Nc2cc(C(N)=O)ccn2)C(c2ccccc2Cl)N=C(Nc2nc3ccc(F)cc3o2)N1 ; CC1=C(C(=O)Nc2cc(C#N)ccn2)C(c2ccccc2Cl)N=C(Nc2nc3ccc(F)cc3o2)N1 ; CC1=C(C(=O)Nc2ccc(C#N)cn2)C(c2ccccc2Cl)N=C(Nc2nc3ccc(F)cc3o2)N1 ; CC1=C(C(=O)Nc2cc(-c3nn[nH]n3)ccn2)C(c2ccccc2Cl)N=C(Nc2nc3ccc(F)cc3o2)N1 ; CC1=C(C(=O)Nc2ccc(CO)cn2)C(c2ccccc2Cl)N=C(Nc2nc3ccc(F)cc3o2)N1]

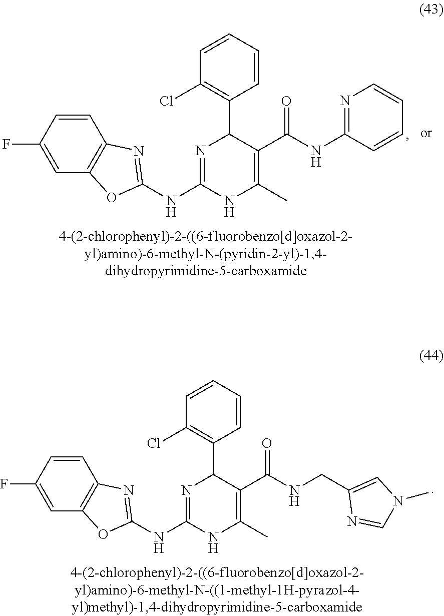 [SMILES: CC1=C(C(=O)Nc2ccccn2)C(c2ccccc2Cl)N=C(Nc2nc3ccc(F)cc3o2)N1 ; "," ; "or" ; CC1=C(C(=O)NCc2cn(C)cn2)C(c2ccccc2Cl)N=C(Nc2nc3ccc(F)cc3o2)N1 ; "."]

14. The compound of claim 1, wherein the compound is:

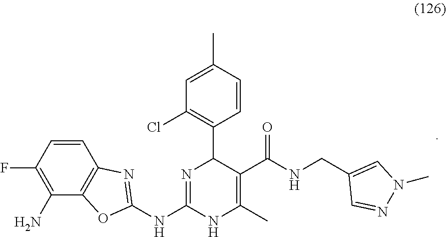 [SMILES: CC1=C(C(=O)NCc2cnn(C)c2)C(c2ccc(C)cc2Cl)N=C(Nc2nc3ccc(F)c(N)c3o2)N1]

15. A composition for inhibiting galactokinase activity comprising Formula II or a salt thereof: 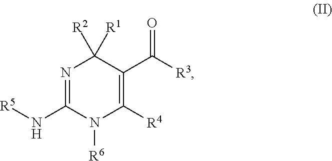
wherein:
R¹ is hydrogen;
R² is selected from:
2-chlorophenyl optionally substituted with one or more substituents, wherein the optional substituents are independently selected from halogen, —CF₃, C₁-C₆-alkyl, C₁-C₄-alkoxy, C₆-C₁₂-aryloxy, —NH₂, —NH(C₁-C₄-alkyl), —N(C₁-C₄-alkyl)₂, C₃-C₆-cycloalkyl, C₄-C₆-heterocycloalkyl, or C₅-C₁₂-heteroaryl; or
phenyl optionally substituted with one or more substituents, wherein the optional substituents are independently selected from —CF₃, —CONH₂, or —SO₂NH₂;
R³ is selected from —NH—C₁-C₄-alkyl-C₅-C₁₂-heteroaryl, —NH—C₁-C₄-alkyl-C₆-C₁₂-aryl, morphilino, or —NR⁷R⁸, wherein R⁷ and R⁸ are each independently selected from hydrogen, C₁-C₆-alkyl, C₃-C₆-cycloalkyl, C₆-C₁₂-aryl, C₅-C₁₂-heteroaryl, CO₂Et-C₁-C₆-alkyl, CO₂H—C₁-C₆-alkyl, NH₂—C₁-C₆-alkyl, NH(C₁-C₄-alkyl)-C₁-C₆-alkyl, N(C₁₋₄-alkyl)₂-C₁-C₆-alkyl, C₁-C₂-alkyloxy-C₁-C₆-alkyl, or HO—C₁-C₆-alkyl; or wherein R⁷ and R⁸ together, including the atoms to which they are attached, form a 5- to 6-membered heteroaryl or a 4- to 6-membered heterocycloalkyl ring, with the proviso that R³ is not —NH-p-tolyl;
R⁴ is selected from hydrogen, C₁-C₆-alkyl, C₅-C₁₂-heteroaryl, C₁-C₄-alkoxy, or C₁-C₂-alkoxy-C₁-C₂-alkyl; or
R³ and R⁴ together form the formula: 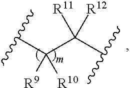
wherein R⁹, R¹⁰, R¹¹, and R¹² are each independently selected from hydrogen, C₁-C₄-alkyl; and m is 0 or 1;
R⁵ is selected from unsubstituted C₅-C₁₂-heteroaryl, optionally substituted benzoxazolyl, or optionally substituted benzothioxazolyl, wherein the optional substituents are independently selected from C₁-C₆-alkyl, C₆-C₁₂-aryl, halogen, —CF₃, C₁-C₄-alkoxy, C₆-C₁₂-aryloxy, —NH₂, —NH(C₁-C₄-alkyl), or —N(C₁-C₄-alkyl)₂; and
R⁶ is selected from C₁-C₆-alkyl, —CH₂CO₂H, —CO₂Et, or —COPh.

16. The composition of claim 15, wherein R² is unsubstituted 2-chlorophenyl and R³ is selected from —NH—C₆-C₁₂-aryl, —NH—C₁-C₂-alkyl-C₆-C₁₂-aryl, —NH—C₅-C₁₂-heteroaryl, or —NH—C₁-C₂-alkyl-C₅-C₁₂-heteroaryl.

17. The composition of claim 15, wherein R² is 2-chlorophenyl and R³ is selected from —NH-4-benzoic acid, —NH-2-isonicotinic acid, or —NH-((1-methyl-1H-pyrazol-4-yl)methyl).

18. The composition of claim 15, wherein the compound is selected from:

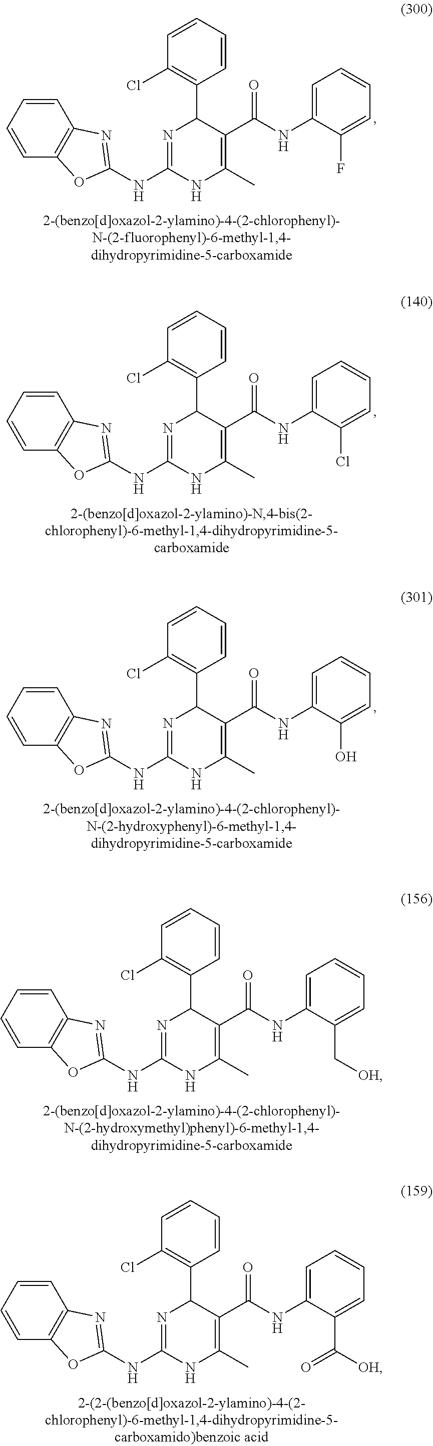

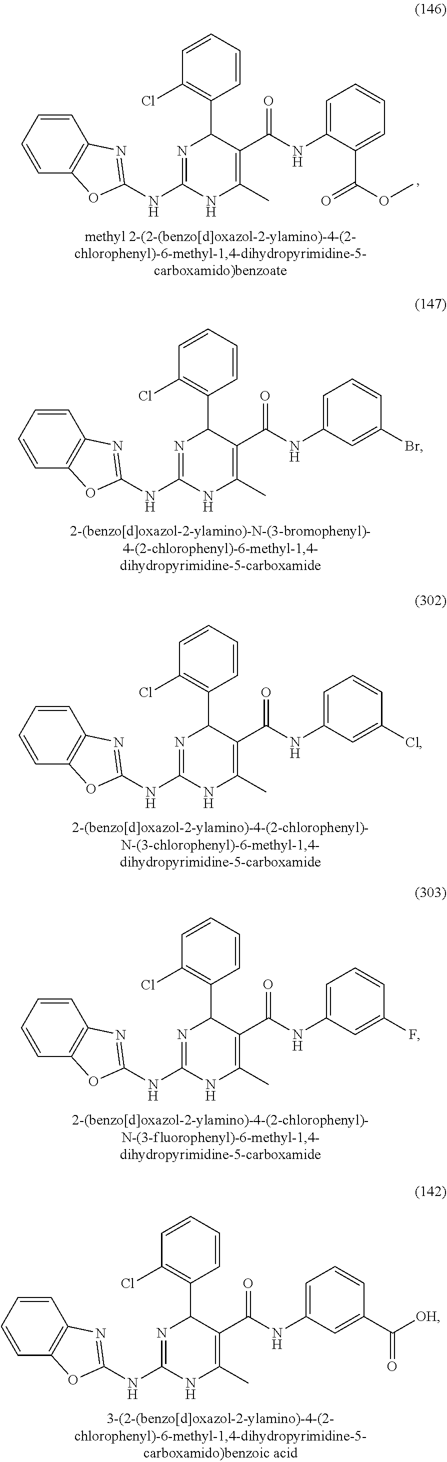 [SMILES: COC(=O)c1ccccc1NC(=O)C1=C(C)NC(Nc2nc3ccccc3o2)=NC1c1ccccc1Cl ; "," ; CC1=C(C(=O)Nc2cccc(Br)c2)C(c2ccccc2Cl)N=C(Nc2nc3ccccc3o2)N1 ; CC1=C(C(=O)Nc2cccc(Cl)c2)C(c2ccccc2Cl)N=C(Nc2nc3ccccc3o2)N1 ; CC1=C(C(=O)Nc2cccc(F)c2)C(c2ccccc2Cl)N=C(Nc2nc3ccccc3o2)N1 ; CC1=C(C(=O)Nc2cccc(C(=O)O)c2)C(c2ccccc2Cl)N=C(Nc2nc3ccccc3o2)N1]

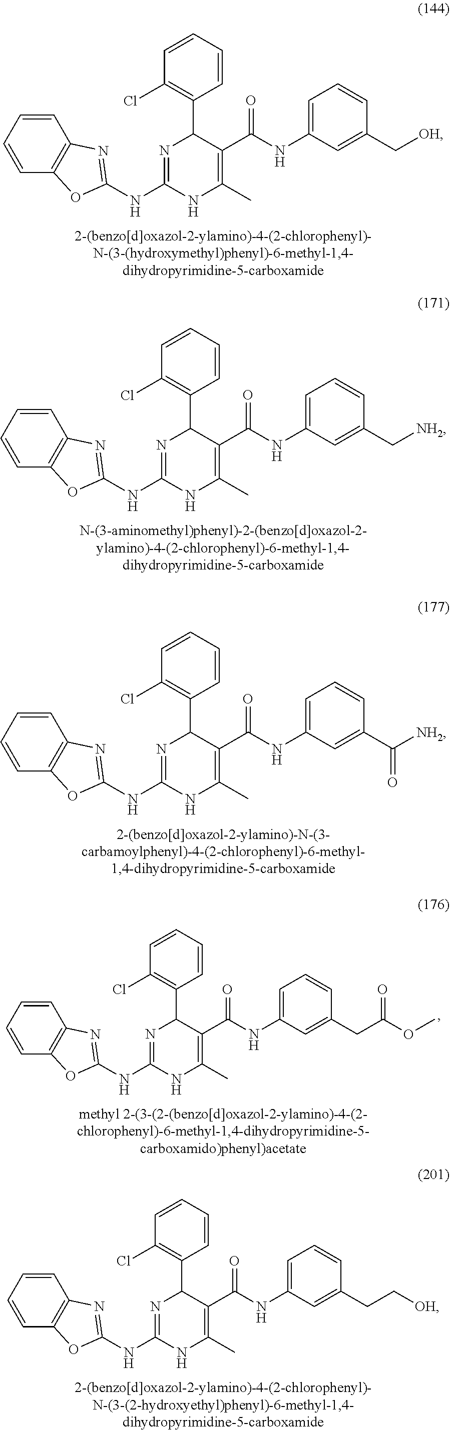 [SMILES: CC1=C(C(=O)Nc2cccc(CO)c2)C(c2ccccc2Cl)N=C(Nc2nc3ccccc3o2)N1 ; CC1=C(C(=O)Nc2cccc(CN)c2)C(c2ccccc2Cl)N=C(Nc2nc3ccccc3o2)N1 ; CC1=C(C(=O)Nc2cccc(C(N)=O)c2)C(c2ccccc2Cl)N=C(Nc2nc3ccccc3o2)N1 ; COC(=O)Cc1cccc(NC(=O)C2=C(C)NC(Nc3nc4ccccc4o3)=NC2c2ccccc2Cl)c1 ; "," ; CC1=C(C(=O)Nc2cccc(CCO)c2)C(c2ccccc2Cl)N=C(Nc2nc3ccccc3o2)N1]

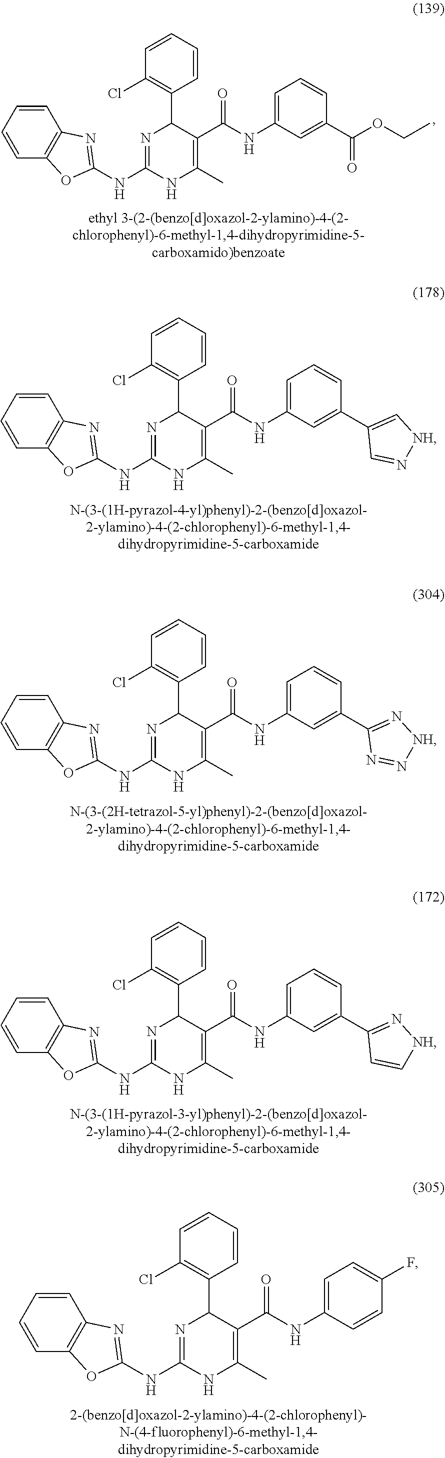 [SMILES: CCOC(=O)c1cccc(NC(=O)C2=C(C)NC(Nc3nc4ccccc4o3)=NC2c2ccccc2Cl)c1 ; "," ; CC1=C(C(=O)Nc2cccc(-c3cn[nH]c3)c2)C(c2ccccc2Cl)N=C(Nc2nc3ccccc3o2)N1 ; CC1=C(C(=O)Nc2cccc(-c3nn[nH]n3)c2)C(c2ccccc2Cl)N=C(Nc2nc3ccccc3o2)N1 ; CC1=C(C(=O)Nc2cccc(-c3cc[nH]n3)c2)C(c2ccccc2Cl)N=C(Nc2nc3ccccc3o2)N1 ; CC1=C(C(=O)Nc2ccc(F)cc2)C(c2ccccc2Cl)N=C(Nc2nc3ccccc3o2)N1]

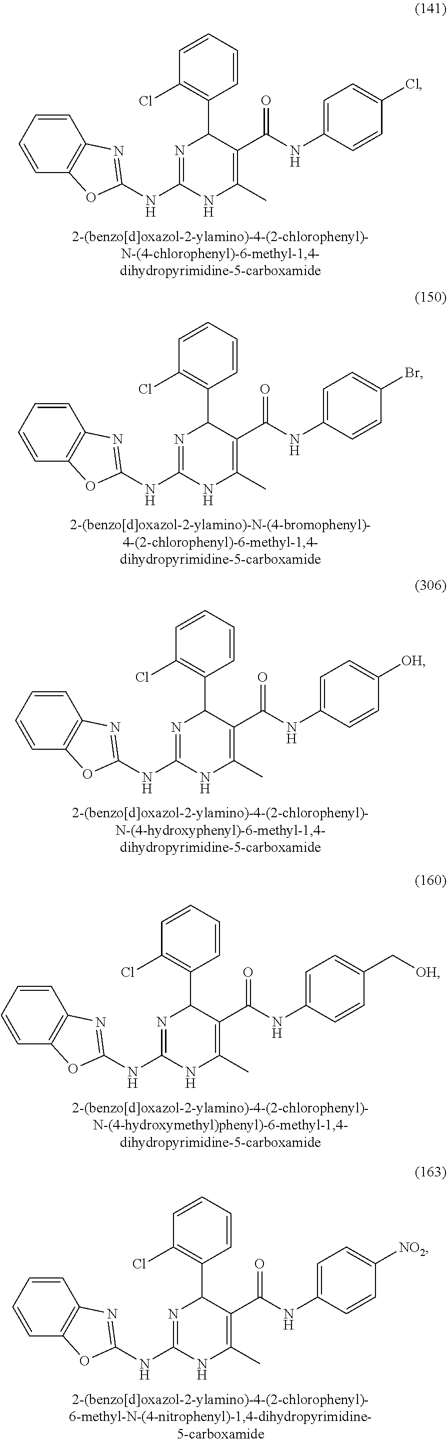 [SMILES: CC1=C(C(=O)Nc2ccc(Cl)cc2)C(c2ccccc2Cl)N=C(Nc2nc3ccccc3o2)N1 ; CC1=C(C(=O)Nc2ccc(Br)cc2)C(c2ccccc2Cl)N=C(Nc2nc3ccccc3o2)N1 ; CC1=C(C(=O)Nc2ccc(O)cc2)C(c2ccccc2Cl)N=C(Nc2nc3ccccc3o2)N1 ; CC1=C(C(=O)Nc2ccc(CO)cc2)C(c2ccccc2Cl)N=C(Nc2nc3ccccc3o2)N1 ; CC1=C(C(=O)Nc2ccc([N+](=O)[O-])cc2)C(c2ccccc2Cl)N=C(Nc2nc3ccccc3o2)N1]

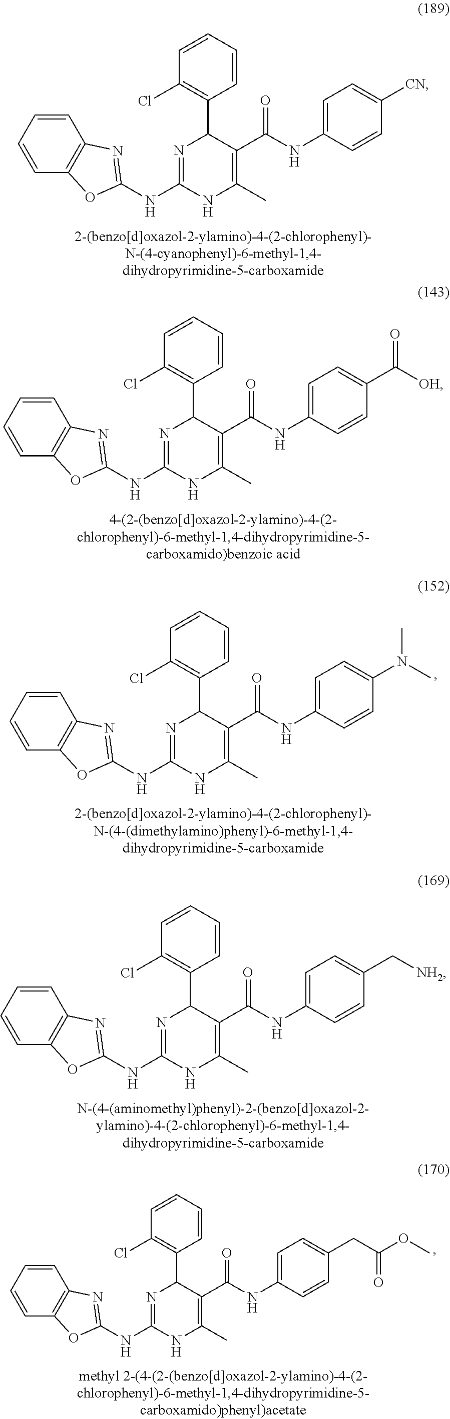

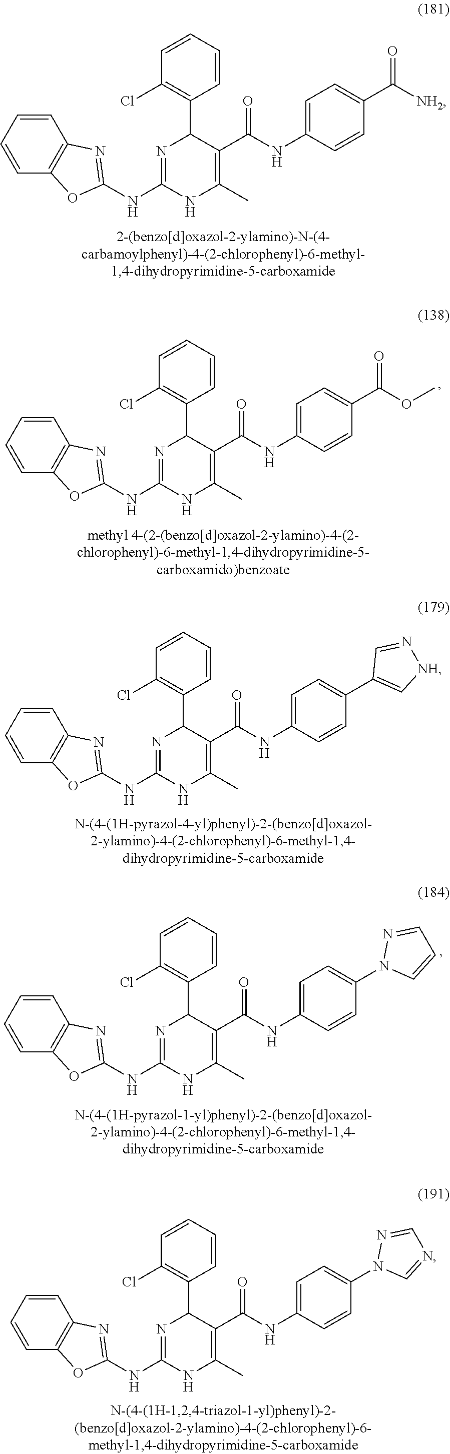

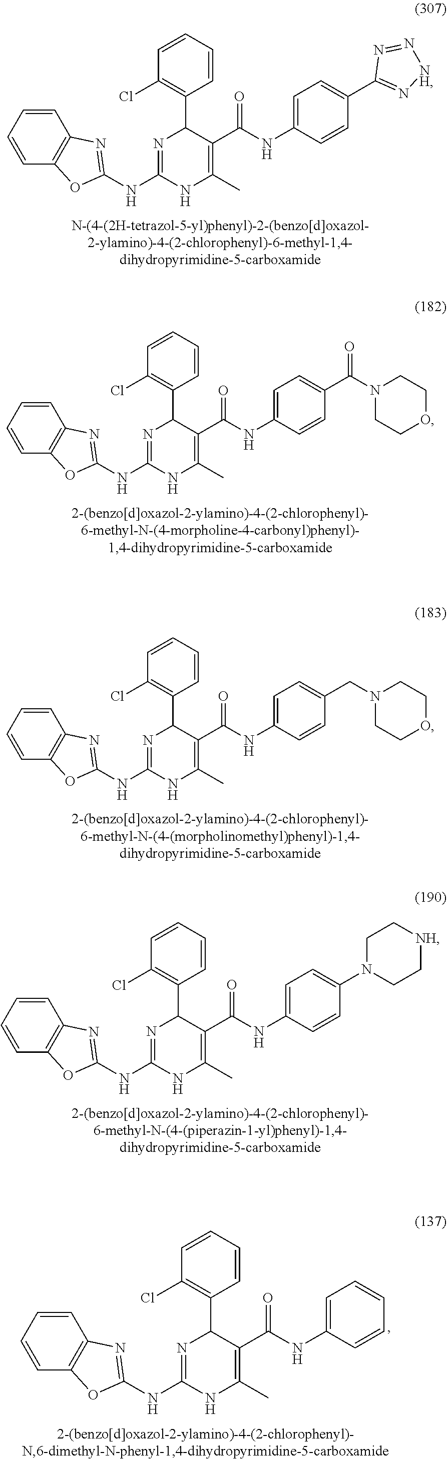 [SMILES: CC1=C(C(=O)Nc2ccc(-c3nn[nH]n3)cc2)C(c2ccccc2Cl)N=C(Nc2nc3ccccc3o2)N1 ; CC1=C(C(=O)Nc2ccc(C(=O)N3CCOCC3)cc2)C(c2ccccc2Cl)N=C(Nc2nc3ccccc3o2)N1 ; CC1=C(C(=O)Nc2ccc(CN3CCOCC3)cc2)C(c2ccccc2Cl)N=C(Nc2nc3ccccc3o2)N1 ; CC1=C(C(=O)Nc2ccc(N3CCNCC3)cc2)C(c2ccccc2Cl)N=C(Nc2nc3ccccc3o2)N1 ; CC1=C(C(=O)Nc2ccccc2)C(c2ccccc2Cl)N=C(Nc2nc3ccccc3o2)N1 ; ","]

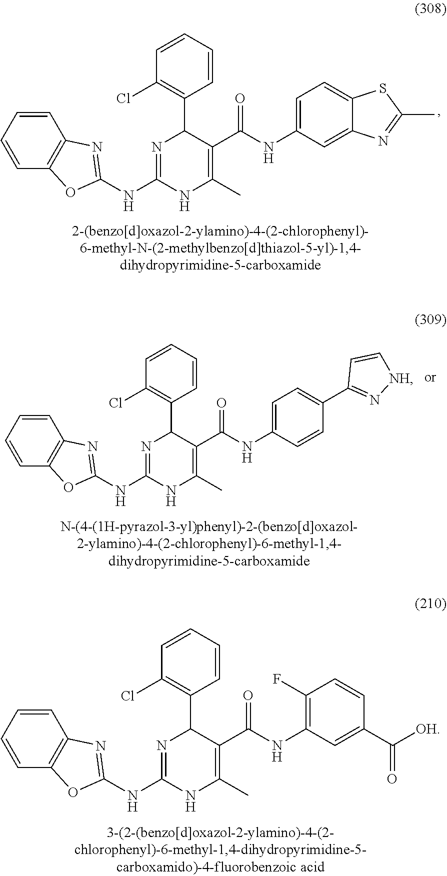 [SMILES: CC1=C(C(=O)Nc2ccc3sc(C)nc3c2)C(c2ccccc2Cl)N=C(Nc2nc3ccccc3o2)N1 ; "," ; CC1=C(C(=O)Nc2ccc(-c3cc[nH]n3)cc2)C(c2ccccc2Cl)N=C(Nc2nc3ccccc3o2)N1 ; "or" ; CC1=C(C(=O)Nc2cc(C(=O)O)ccc2F)C(c2ccccc2Cl)N=C(Nc2nc3ccccc3o2)N1]

19. The composition of claim 15, wherein the compound is selected from:

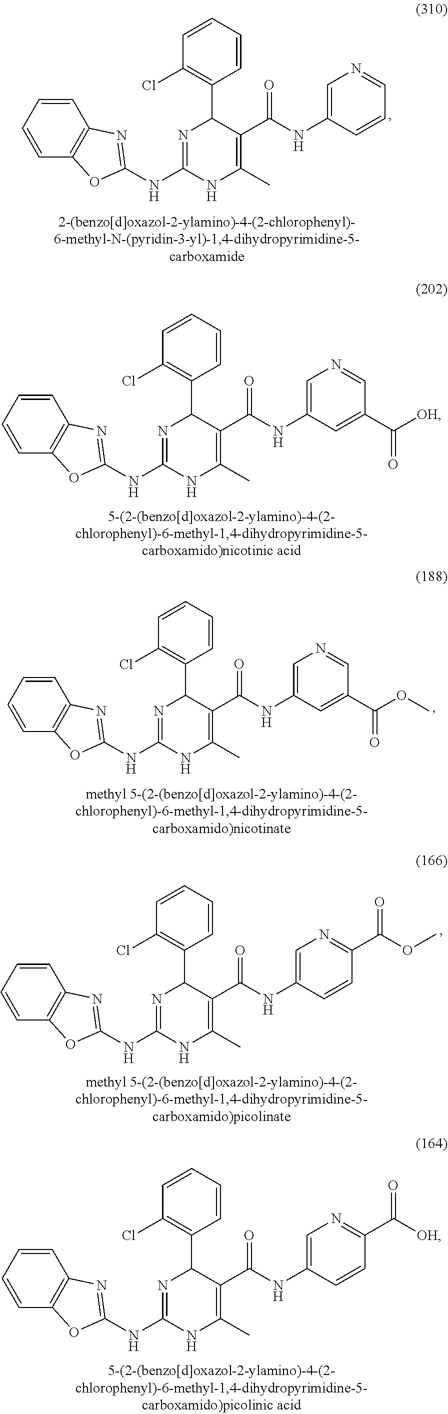

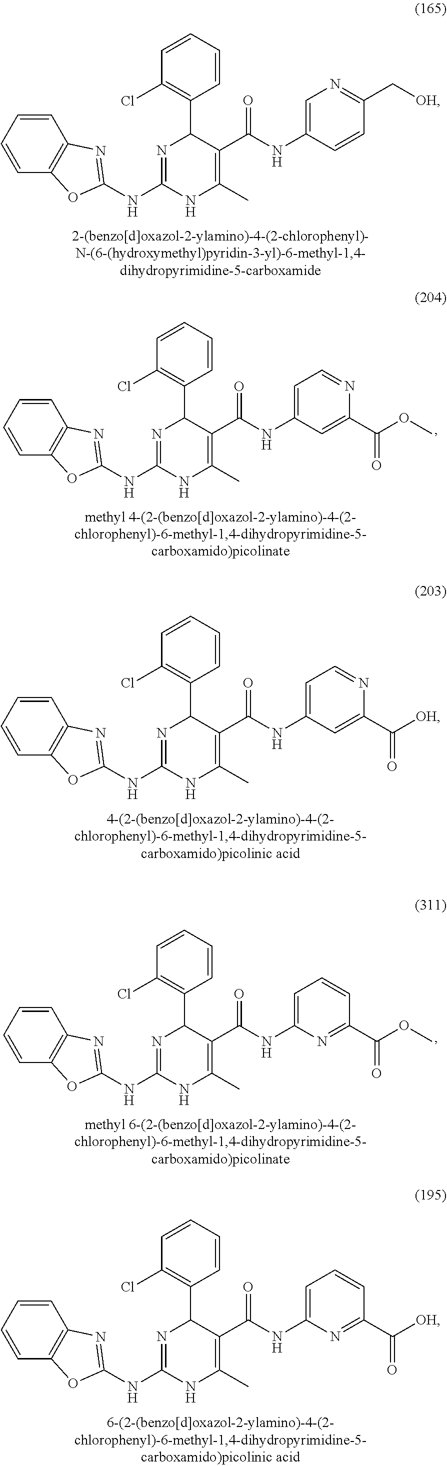

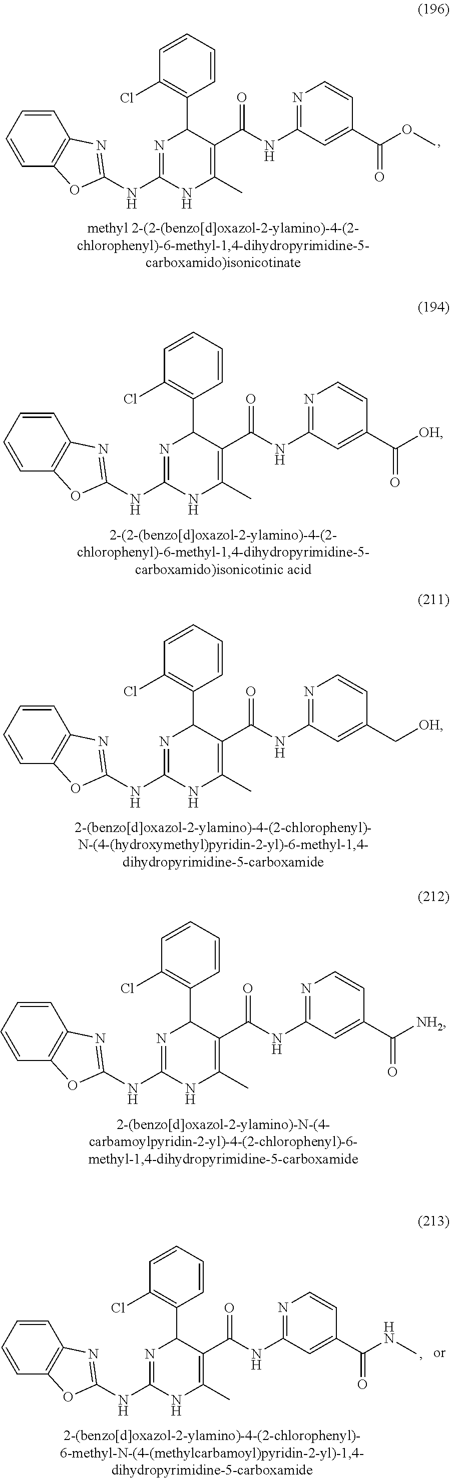

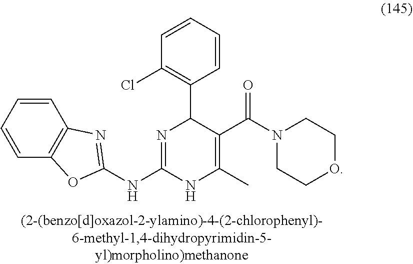 [SMILES: CC1=C(C(=O)N2CCOCC2)C(c2ccccc2Cl)N=C(Nc2nc3ccccc3o2)N1]

20. The composition of claim 15, wherein the compound is selected from:

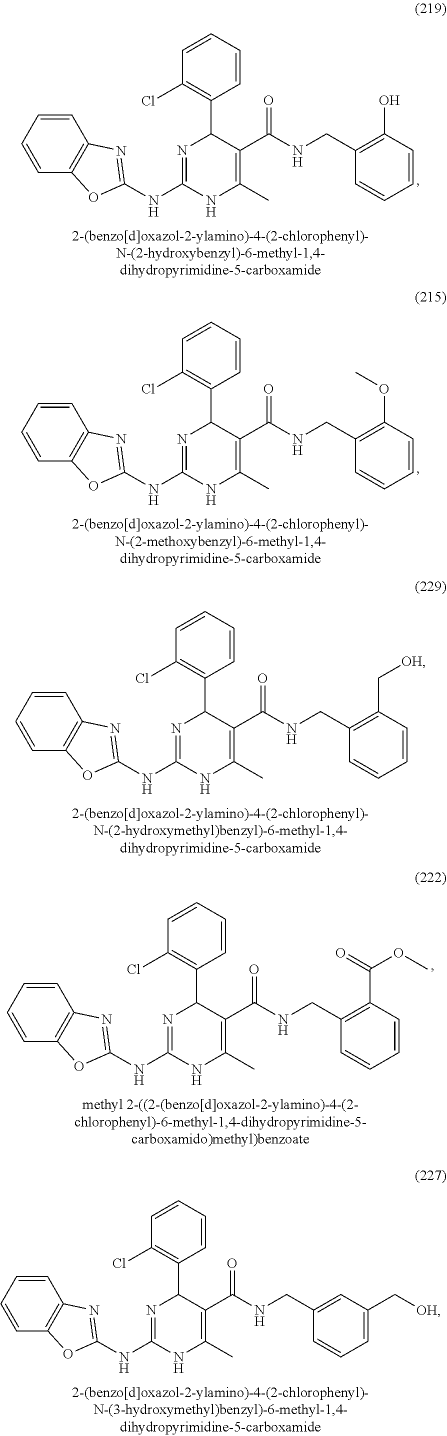

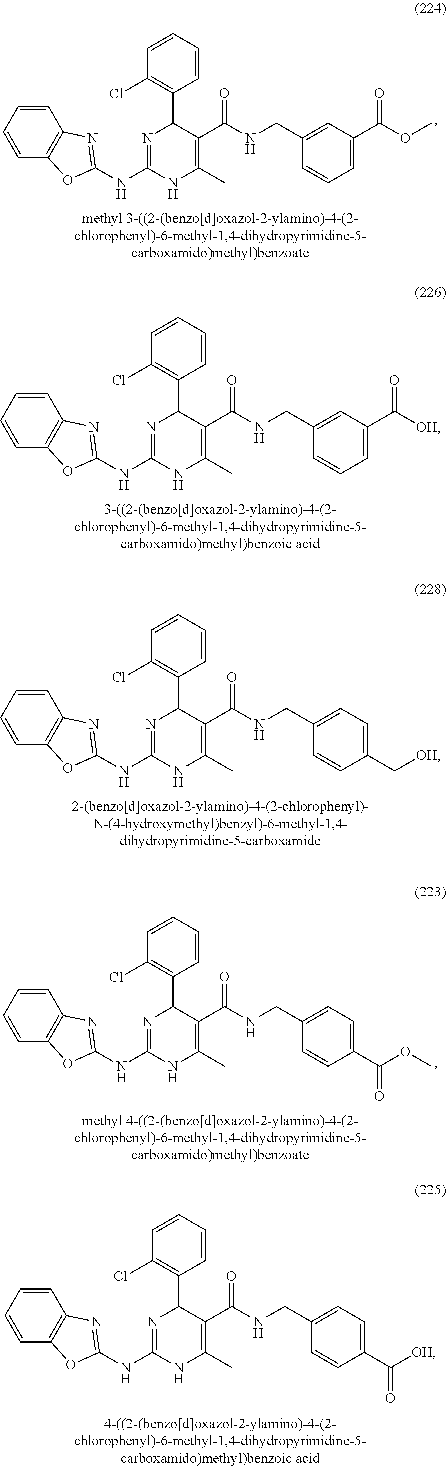

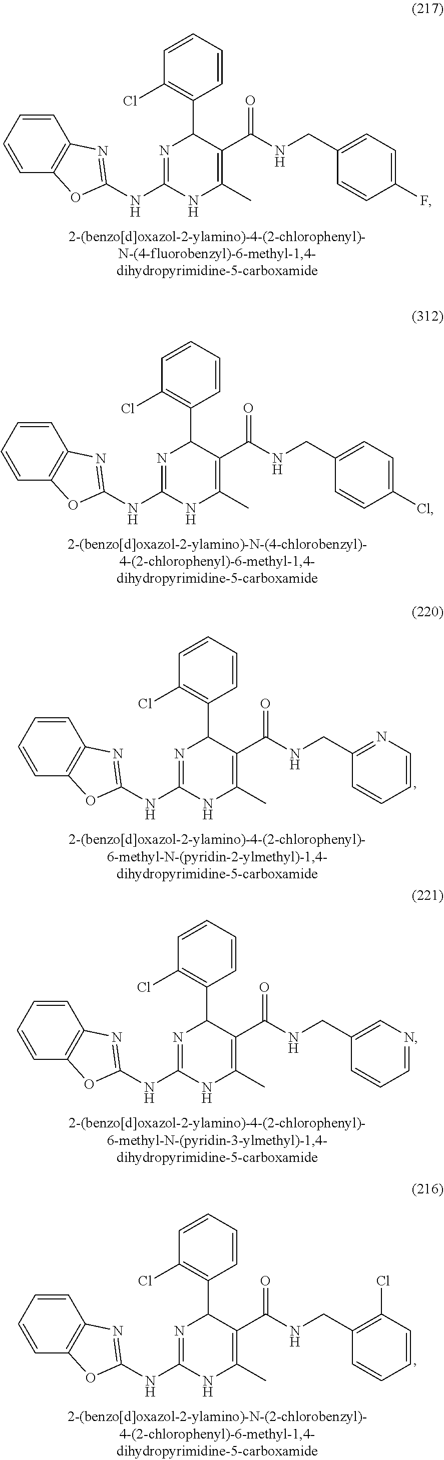

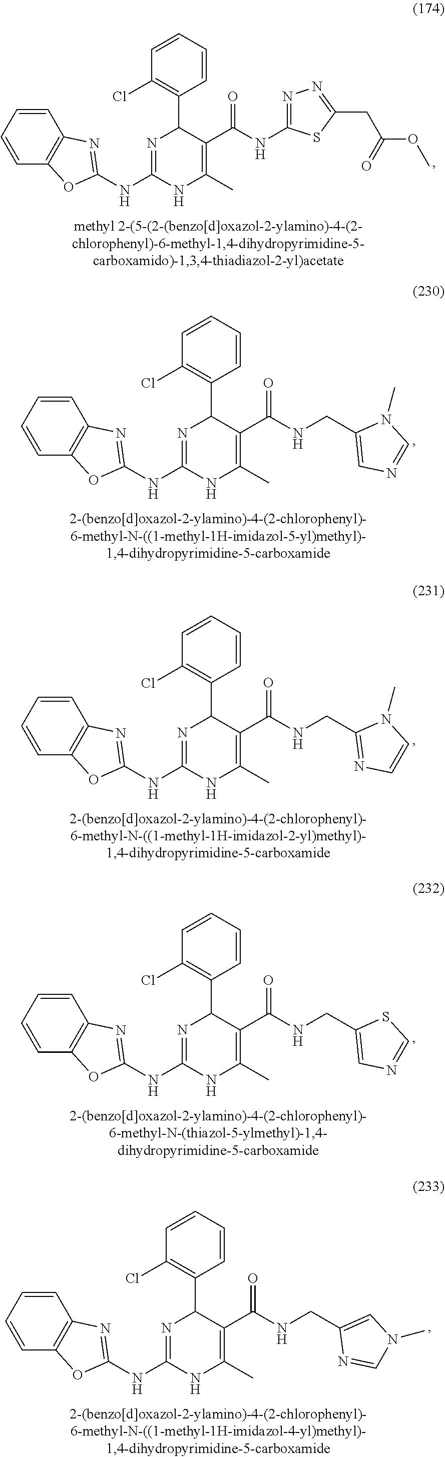

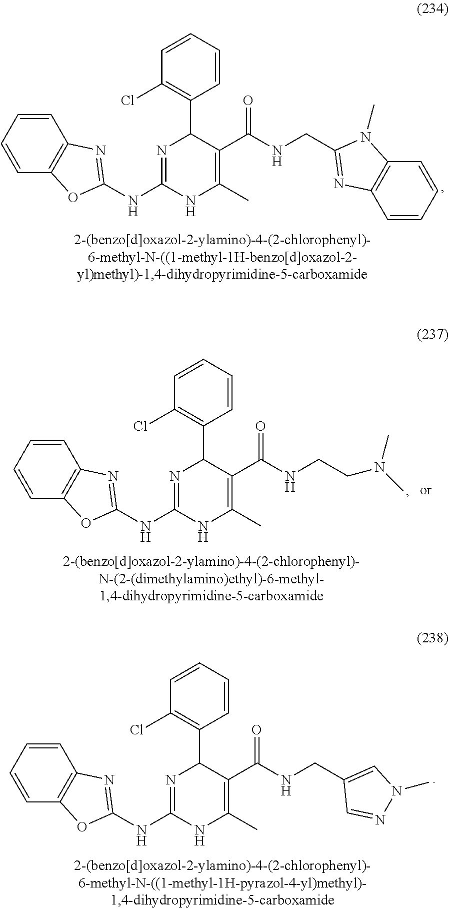

21. The composition of claim 15, wherein the compound is selected from:

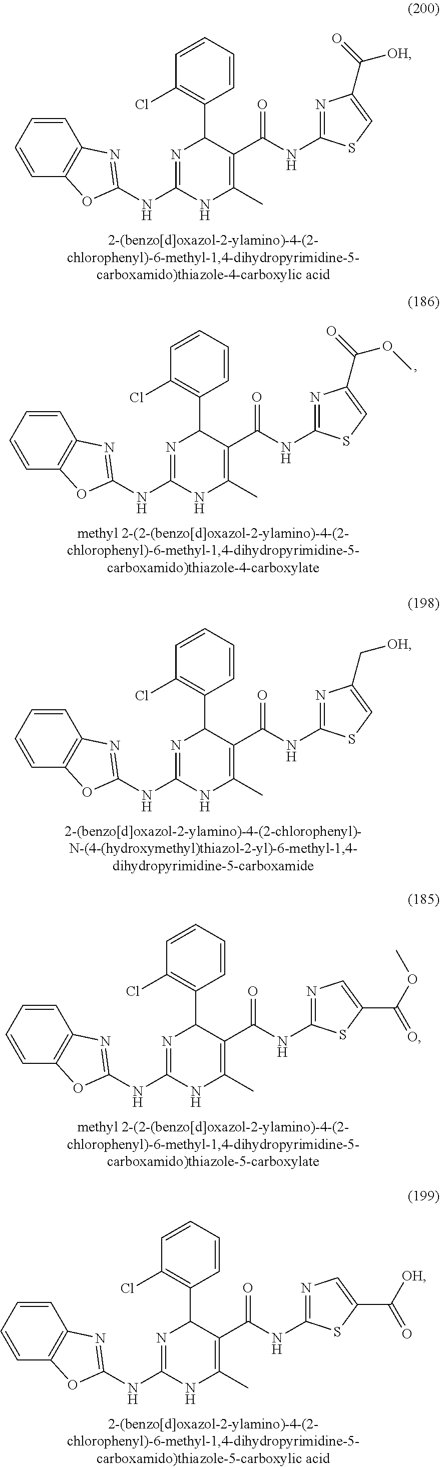 [SMILES: CC1=C(C(=O)Nc2nc(C(=O)O)cs2)C(c2ccccc2Cl)N=C(Nc2nc3ccccc3o2)N1 ; COC(=O)c1csc(NC(=O)C2=C(C)NC(Nc3nc4ccccc4o3)=NC2c2ccccc2Cl)n1 ; "," ; CC1=C(C(=O)Nc2nc(CO)cs2)C(c2ccccc2Cl)N=C(Nc2nc3ccccc3o2)N1 ; COC(=O)c1cnc(NC(=O)C2=C(C)NC(Nc3nc4ccccc4o3)=NC2c2ccccc2Cl)s1 ; CC1=C(C(=O)Nc2ncc(C(=O)O)s2)C(c2ccccc2Cl)N=C(Nc2nc3ccccc3o2)N1]

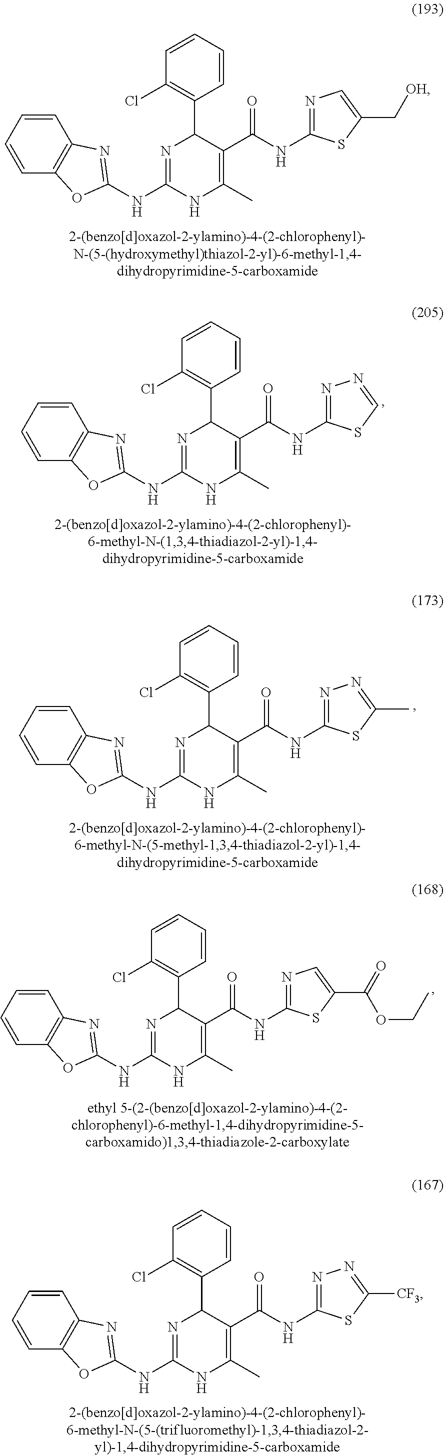

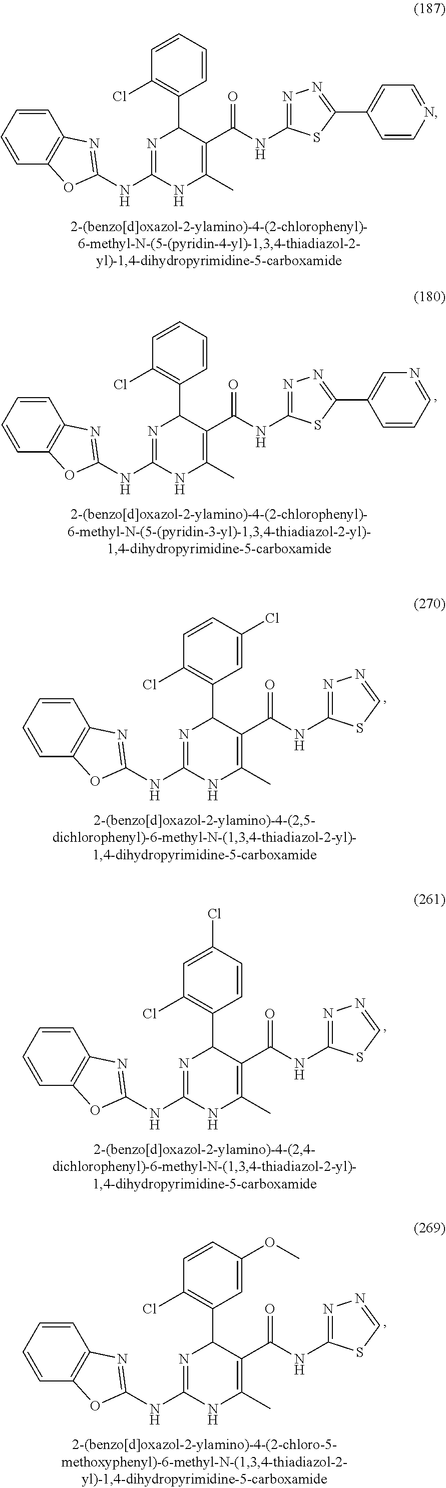

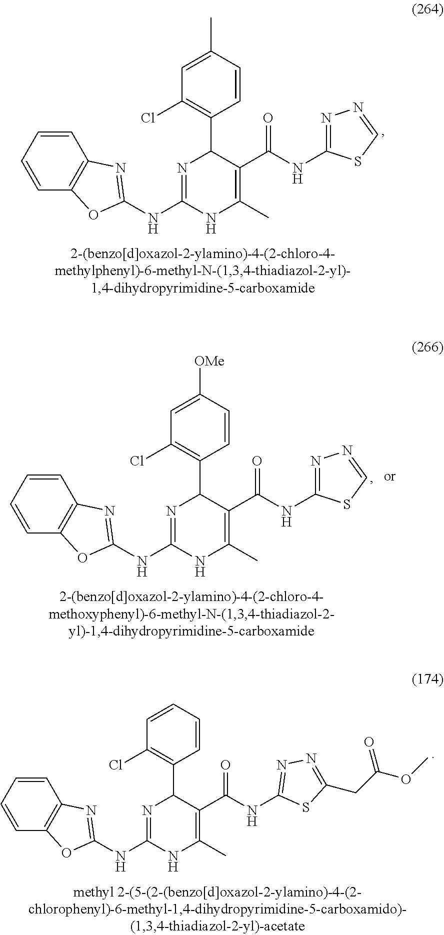

22. The composition of claim 15, wherein the compound is selected from:

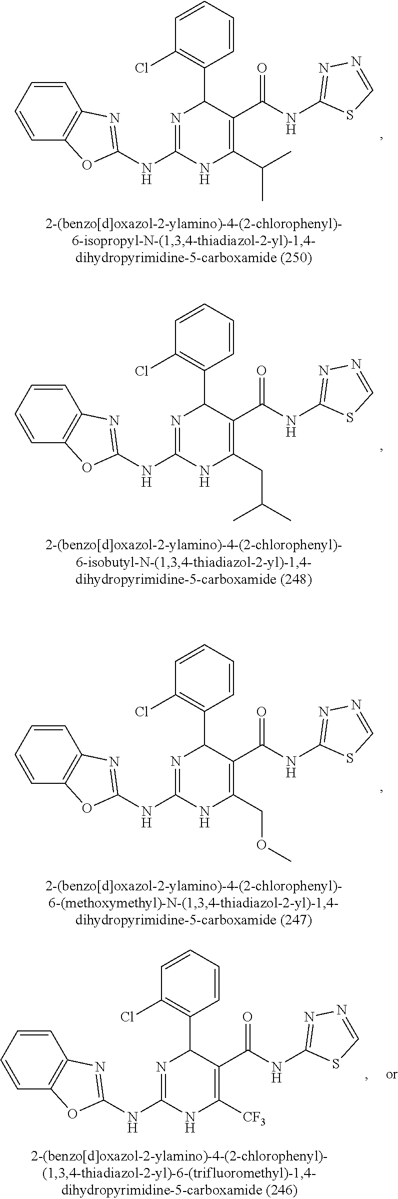 [SMILES: CC(C)C1=C(C(=O)Nc2nncs2)C(c2ccccc2Cl)N=C(Nc2nc3ccccc3o2)N1 ; CC(C)CC1=C(C(=O)Nc2nncs2)C(c2ccccc2Cl)N=C(Nc2nc3ccccc3o2)N1 ; COCC1=C(C(=O)Nc2nncs2)C(c2ccccc2Cl)N=C(Nc2nc3ccccc3o2)N1 ; O=C(Nc1nncs1)C1=C(C(F)(F)F)NC(Nc2nc3ccccc3o2)=NC1c1ccccc1Cl]

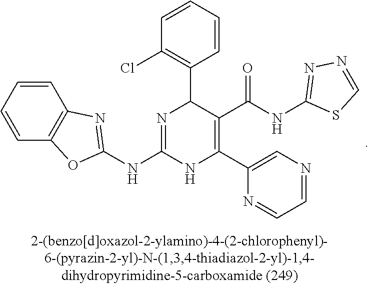 [SMILES: O=C(Nc1nncs1)C1=C(c2cnccn2)NC(Nc2nc3ccccc3o2)=NC1c1ccccc1Cl]

23. The composition of claim 15, wherein the compound is selected from:

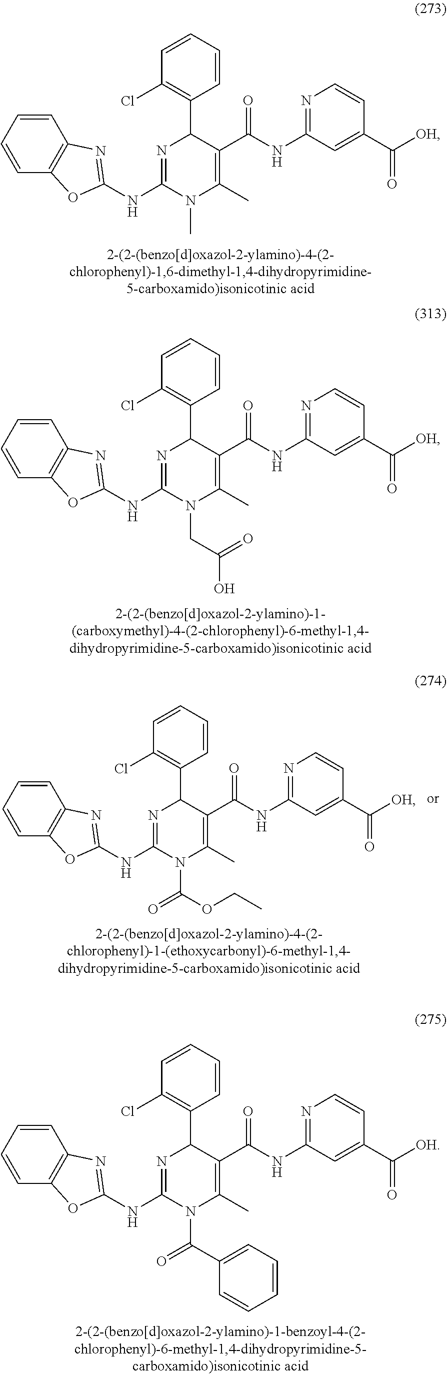 [SMILES: CC1=C(C(=O)Nc2cc(C(=O)O)ccn2)C(c2ccccc2Cl)N=C(Nc2nc3ccccc3o2)N1C ; CC1=C(C(=O)Nc2cc(C(=O)O)ccn2)C(c2ccccc2Cl)N=C(Nc2nc3ccccc3o2)N1CC(=O)O ; CCOC(=O)N1C(Nc2nc3ccccc3o2)=NC(c2ccccc2Cl)C(C(=O)Nc2cc(C(=O)O)ccn2)=C1C ; "or" ; CC1=C(C(=O)Nc2cc(C(=O)O)ccn2)C(c2ccccc2Cl)N=C(Nc2nc3ccccc3o2)N1C(=O)c1ccccc1]

24. The composition of claim 15, wherein the compound is selected from:

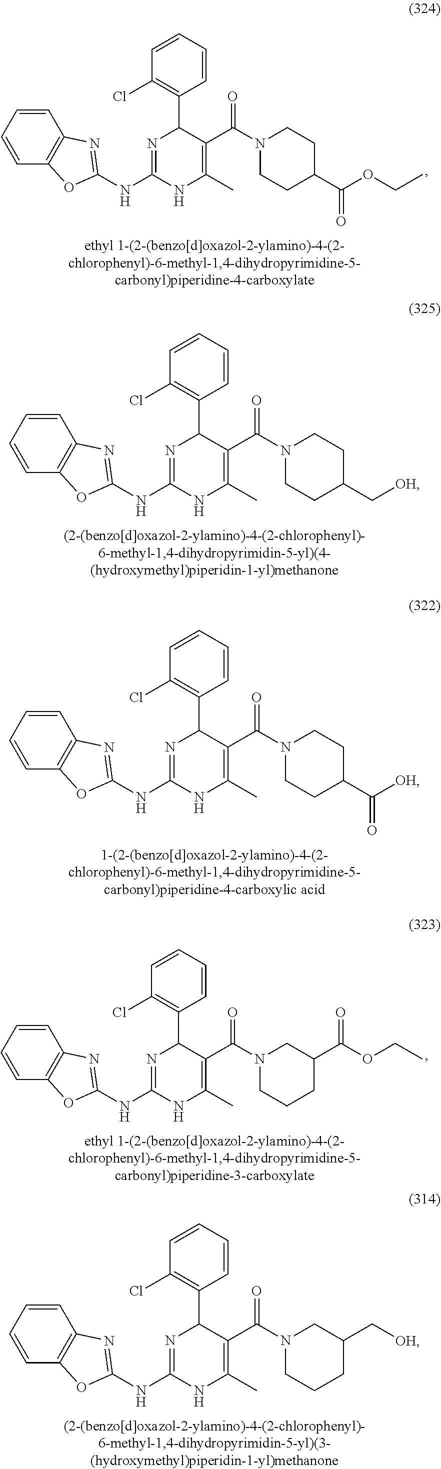

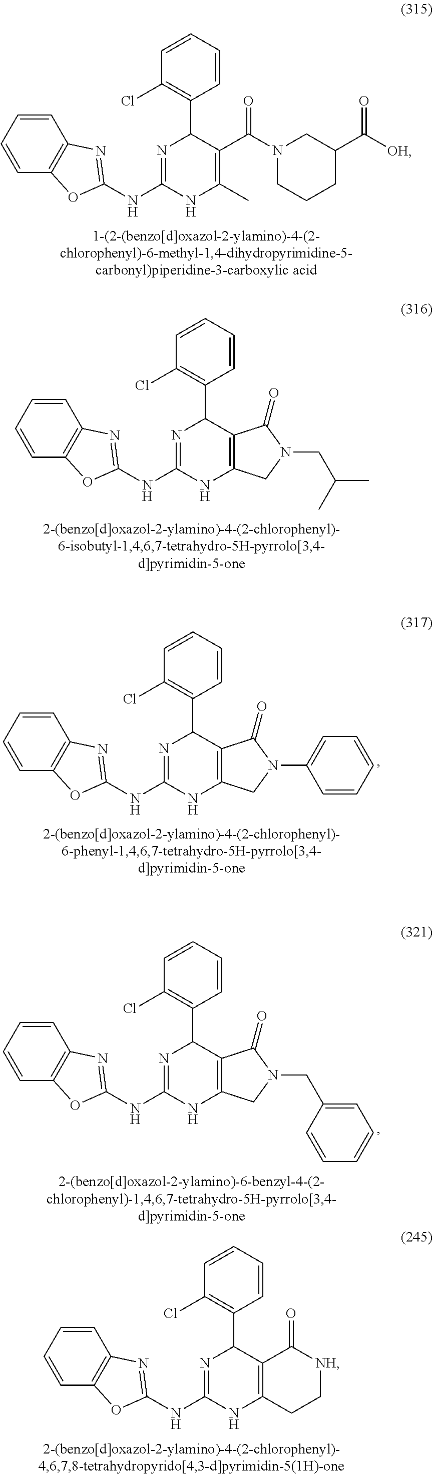

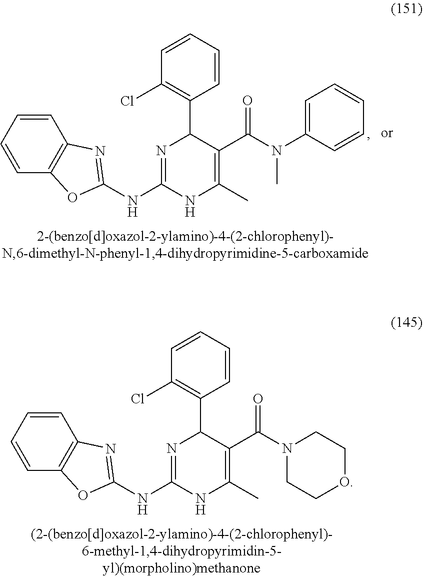 [SMILES: CC1=C(C(=O)N(C)c2ccccc2)C(c2ccccc2Cl)N=C(Nc2nc3ccccc3o2)N1 ; "," ; "or" ; CC1=C(C(=O)N2CCOCC2)C(c2ccccc2Cl)N=C(Nc2nc3ccccc3o2)N1]

25. A composition for inhibiting galactokinase activity comprising Formula III or a salt thereof: 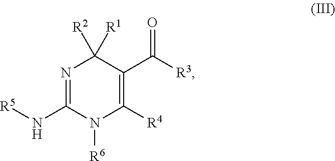
wherein:
R¹ and R² are each independently selected from hydrogen, C₁-C₄-alkyl, C₁-C₂-alkoxy, C₁-C₂-hydroxy, C₁-C₂-thioalkyl, C₆-C₁₂-aryl, or C₅-C₁₂-heteroaryl; or
R¹ and R² taken together, including the atoms to which they are attached, form a 4- to 8-membered carbocycle or a 4- to 6-membered heterocycle;
R³ is selected from —NH—C₅-C₁₂-heteroaryl, —NH—C₁-C₂-alkyl-C₅-C₁₂-heteroaryl, or —NH—C₁-C₂-alkyl-C₃-C₆-heterocycloalkyl;
R⁴ is C₁-C₆-alkyl;
R⁵ is selected from unsubstituted C₅-C₁₂-heteroaryl, optionally substituted benzoxazolyl, or optionally substituted benzothioxazolyl, wherein the optional substituents are selected from C₁-C₆-alkyl, C₆-C₁₂-aryl, halogen, —CF₃, C₁-C₄-alkoxy, C₆-C₁₂-aryloxy, —NH₂, —NH(C₁-C₄-alkyl), or —N(C₁-C₄-alkyl)₂; and
R⁶ is selected from hydrogen or C₁-C₆-alkyl.

26. The composition of claim 25, wherein the compound is selected from:

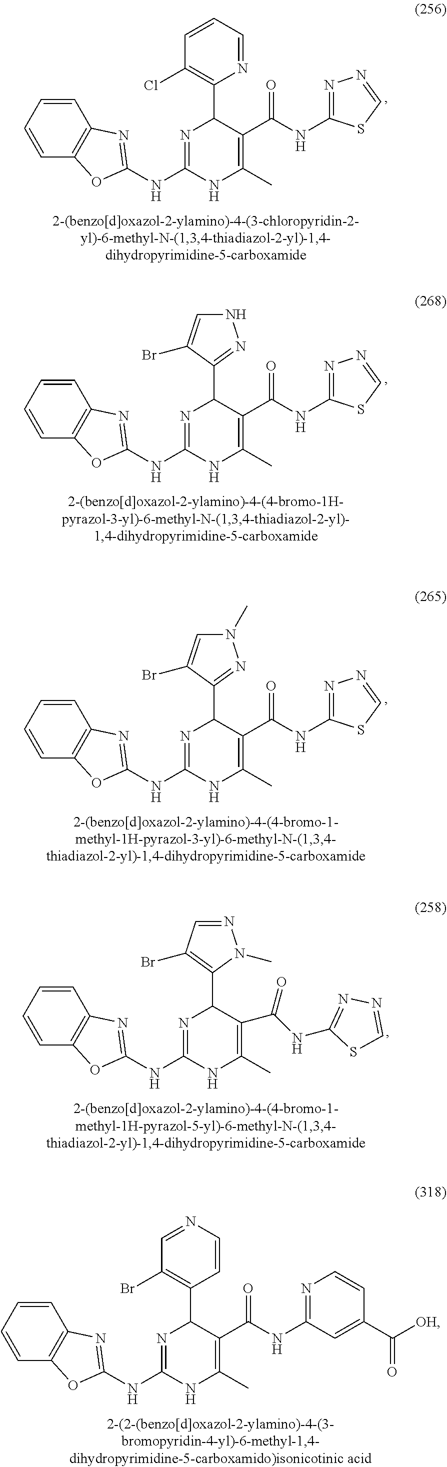

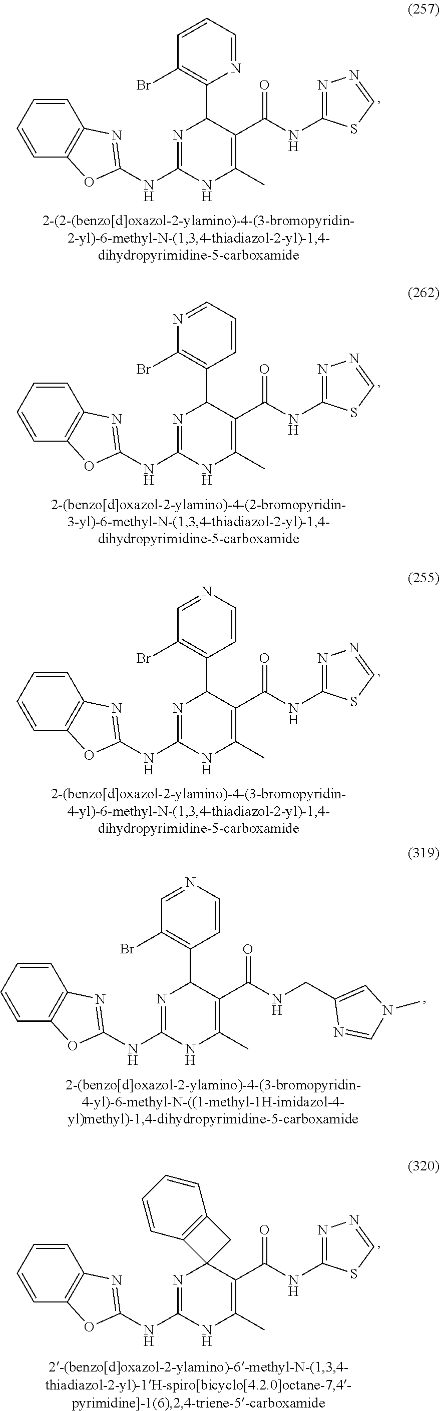

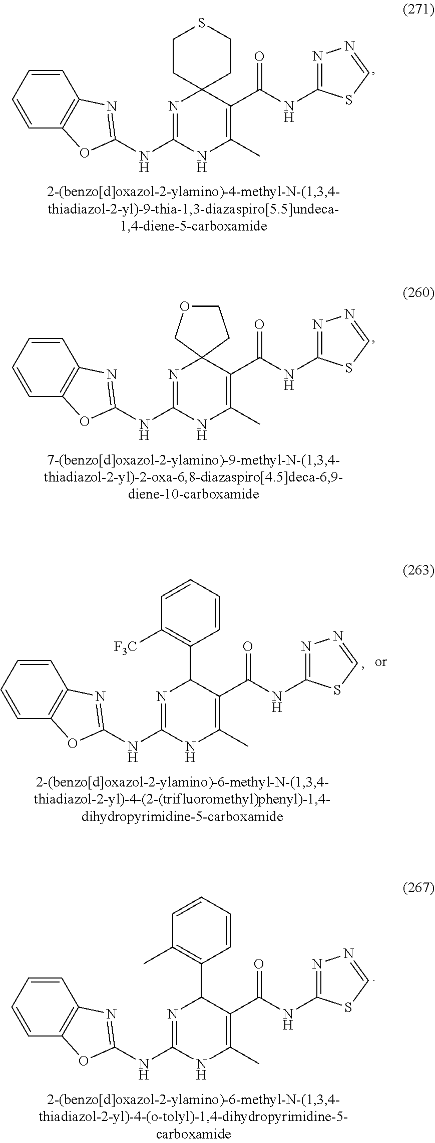

27. A compound selected from:

| | |
|---|---|
| (1) | 2-(4-(2-chlorophenyl)-2-((6- |
| | fluorobenzo[d]oxazol-2-yl)amino)-6-methyl- |
| | 1,4-dihydropyrimidine-5- |
| | carboxamido)isonicotinic acid; |
| (2) | 2-(2-((4-aminobenzo[d]oxazol-2-yl)amino)-4- |
| | (2-chlorophenyl)-6-methyl-1,4- |
| | dihydropyrimidine-5- |
| | carboxamido)isonicotinic acid; |
| (3) | 2-(2-((7-aminobenzo[d]oxazol-2-yl)amino)-4- |
| | (2-chlorophenyl)-6-methyl-1,4- |
| | dihydropyrimidine-5- |
| | carboxamido)isonicotinic acid; |
| (4) | 2-(4-(2-chlorophenyl)-6-methyl-2-((5- |
| | methylbenzo[d]oxazol-2-yl)amino)-1,4- |
| | dihydropyrimidine-5- |
| | carboxamido)isonicotinic acid; |
| (5) | 2-(4-(2-chlorophenyl)-2-((5- |
| | methoxybenzo[d]oxazol-2-yl)amino)-6- |
| | methyl-1,4-dihydropyrimidine-5- |
| | carboxamido)isonicotinic acid; |
| (6) | 2-(4-(2-chlorophenyl)-6-methyl-2-((6- |
| | methylbenzo[d]oxazol-2-yl)amino)-1,4- |
| | dihydropyrimidine-5- |
| | carboxamido)isonicotinic acid; |
| (7) | 2-(4-(2-chlorophenyl)-6-methyl-2-((6- |
| | methoxybenzo[d]oxazol-2-yl)amino)-1,4- |
| | dihydropyrimidine-5- |
| | carboxamido)isonicotinic acid; |
| (8) | 2-(4-(2-chlorophenyl)-6-methyl-2-((4- |
| | fluorobenzo[d]oxazol-2-yl)amino)-1,4- |
| | dihydropyrimidine-5- |
| | carboxamido)isonicotinic acid; |
| (9) | 2-(2-((5-chlorobenzo[d]oxazol-2-yl)amino)-4- |
| | (2-chlorophenyl)-6-methyl-1,4- |
| | dihydropyrimidine-5- |
| | carboxamido)isonicotinic acid; |
| (10) | 2-(4-(2-chlorophenyl)-2-((7- |
| | fluorobenzo[djoxazol-2-yl)amino)-6-methyl- |
| | 1,4-dihydropyrimidine-5- |
| | carboxamido)isonicotinic acid; |
| (11) | 2-(2-((4-chlorobenzo[d]oxazol-2-yl)amino)-4- |
| | (2-chlorophenyl)-6-methyl-1,4- |
| | dihydropyrimidine-5- |
| | carboxamido)isonicotinic acid; |
| (12) | 2-(2-((5-aminobenzo[d]oxazol-2-yl)amino)-4- |
| | (2-chlorophenyl)-6-methyl-1,4- |
| | dihydropyrimidine-5- |
| | carboxamido)isonicotinic acid; |
| (13) | 2-(4-(2-chlorophenyl)-2-((5- |
| | fluorobenzo[djoxazol-2-yl)amino)-6-methyl- |
| | 1,4-dihydropyrimidine-5- |
| | carboxamido)isonicotinic acid; |
| (14) | 2-(2-((6-chlorobenzo[d]oxazol-2-yl)amino)-4- |
| | (2-chlorophenyl)-6-methyl-1,4- |
| | dihydropyrimidine-5- |
| | carboxamido)isonicotinic acid; |
| (15) | 2-(4-(2-chlorophenyl)-6-methyl-2-((7- |
| | methylbenzo[d]oxazol-2-yl)amino)-1,4- |
| | dihydropyrimidine-5- |
| | carboxamido)isonicotinic acid; |
| (16) | 2-(4-(2-chlorophenyl)-6-methyl-2-((7- |
| | (trifluoromethyl)benzo[d]oxazol-2-yl)amino)- |
| | 1,4-dihydropyrimidine-5- |
| | carboxamido)isonicotinic acid; |
| (17) | 2-(2-((7-chlorobenzo[d]oxazol-2-yl)amino)-4- |
| | (2-chlorophenyl)-6-methyl-1,4- |
| | dihydropyrimidine-5- |
| | carboxamido)isonicotinic acid; |
| (18) | 2-(4-(2-chlorophenyl)-6-methyl-2-((4- |
| | methylbenzo[d]oxazol-2-yl)amino)-1,4- |
| | dihydropyrimidine-5- |
| | carboxamido)isonicotinic acid; |
| (19) | (R)-2-(4-(2-chlorophenyl)-2-((6- |
| | fluorobenzo[d]oxazol-2-yl)amino)-6-methyl- |
| | 1,4-dihydropyrimidine-5- |
| | carboxamido)isonicotinic acid; |
| (20) | (S)-2-(4-(2-chlorophenyl)-2-((6- |
| | fluorobenzo[d]oxazol-2-yl)amino)-6-methyl- |
| | 1,4-dihydropyrimidine-5- |
| | carboxamido)isonicotinic acid; |
| (21) | 2-(4-(2-chlorophenyl)-6-methyl-2-((7- |
| | (trifluoromethoxy)benzo[d]oxazol-2- |
| | yl)amino)-1,4-dihydropyrimidine-5- |
| | carboxamido)isonicotinic acid; |
| (22) | 2-(4-(2-chlorophenyl)-2-((7- |
| | methoxybenzo[d]oxazol-2-yl)amino)-6- |
| | methyl-1,4-dihydropyrimidine-5- |
| | carboxamido)isonicotinic acid; |
| (23) | 2-(4-(2-chlorophenyl)-6-methyl-2-((7- |
| | nitrobenzo[d]oxazol-2-yl)amino)-1,4- |
| | dihydropyrimidine-5- |
| | carboxamido)isonicotinic acid; |
| (24) | (R)-2-(2-((7-aminobenzo[d]oxazol-2- |
| | yl)amino)-4-(2-chlorophenyl)-6-methyl-1,4- |
| | dihydropyrimidine-5- |
| | carboxamido)isonicotinic acid; |
| (25) | (S)-2-(2-((7-aminobenzo[d]oxazol-2- |
| | yl)amino)-4-(2-chlorophenyl)-6-methyl-1,4- |
| | dihydropyrimidine-5- |
| | carboxamido)isonicotinic acid; |
| (26) | 6-(4-(2-chlorophenyl)-2-((6- |
| | fluorobenzo[djoxazol-2-yl)amino)-6-methyl- |
| | 1,4-dihydropyrimidine-5- |
| | carboxamido)nicotinic acid; |
| (27) | 3-(4-(2-chlorophenyl)-2-((6- |
| | fluorobenzo[djoxazol-2-yl)amino)-6-methyl- |
| | 1,4-dihydropyrimidine-5-carboxamido)-4- |
| | methoxybenzoic acid; |
| (28) | 3-(4-(2-chlorophenyl)-2-((6- |
| | fluorobenzo[djoxazol-2-yl)amino)-6-methyl- |
| | 1,4-dihydropyrimidine-5-carboxamido)-4- |
| | methylbenzoic acid; |
| (29) | 4-chloro-3-(4-(2-chlorophenyl)-2-((6- |
| | fluorobenzo[d]oxazol-2-yl)amino)-6-methyl- |
| | 1,4-dihydropyrimidine-5- |
| | carboxamido)benzoic acid; |
| (30) | 3-(4-(2-chlorophenyl)-2-((6- |
| | fluorobenzo[djoxazol-2-yl)amino)-6-methyl- |
| | 1,4-dihydropyrimidine-5-carboxamido)-4- |
| | fluorobenzoic acid; |
| (31) | (R)-methyl 2-(4-(2-chlorophenyl)-2-((6- |
| | fluorobenzo[d]oxazol-2-yl)amino)-6-methyl- |
| | 1,4-dihydropyrimidine-5- |
| | carboxamido)isonicotinate; |
| (32) | (S)-methyl 2-(4-(2-chlorophenyl)-2-((6- |
| | fluorobenzo[d]oxazol-2-yl)amino)-6-methyl- |
| | 1,4-dihydropyrimidine-5- |
| | carboxamido)isonicotinate; |
| (33) | 4-(2-chlorophenyl)-2-((6- |
| | fluorobenzo[d]oxazol-2-yl)amino)-6-methyl- |
| | N-(2-(methylamino)ethyl)-1,4- |
| | dihydropyrimidine-5-carboxamide; |
| (34) | 4-(2-chlorophenyl)-2-((6- |
| | fluorobenzo[d]oxazol-2-yl)amino)-6-methyl- |
| | N-((1-methyl-1H-imidazol-4-yl)methyl)-1,4- |
| | dihydropyrimidine-5-carboxamide; |
| (35) | N-(5-(2H-tetrazol-5-yl)pyridin-2-yl)-4-(2- |
| | chlorophenyl)-2-((6-fluorobenzo[d]oxazol-2- |
| | yl)amino)-6-methyl-1,4-dihydropyrimidine-5- |
| | carboxamide; |
| (36) | N-(5-carbamoylpyridin-2-yl)-4-(2- |
| | chlorophenyl)-2-((6-fluorobenzo[d]oxazol-2- |
| | yl)amino)-6-methyl-1,4-dihydropyrimidine-5- |
| | carboxamide; |
| (37) | 4-(2-chlorophenyl)-2-((6- |
| | fluorobenzo[d]oxazol-2-yl)amino)-N-(4- |
| | (hydroxymethyl)pyridin-2-yl)-6-methyl-1,4- |
| | dihydropyrimidine-5-carboxamide; |
| (38) | N-(4-carbamoylpyridin-2-yl)-4-(2- |
| | chlorophenyl)-2-((6-fluorobenzo[d]oxazol-2- |
| | yl)amino)-6-methyl-1,4-dihydropyrimidine-5- |
| | carboxamide; |
| (39) | 4-(2-chlorophenyl)-N-(4-cyanopyridin-2-yl)- |
| | 2-((6-fluorobenzo[d]oxazol-2-yl)amino)-6- |
| | methyl-1,4-dihydropyrimidine-5- |
| | carboxamide; |
| (40) | 4-(2-chlorophenyl)-N-(5-cyanopyridin-2-yl)- |
| | 2-((6-fluorobenzo[d]oxazol-2-yl)amino)-6- |
| | methyl-1,4-dihydropyrimidine-5- |
| | carboxamide; |
| (41) | N-(4-(2H-tetrazol-5-yl)pyridin-2-yl)-4-(2- |
| | chlorophenyl)-2-((6-fluorobenzo[d]oxazol-2- |
| | yl)amino)-6-methyl-1,4-dihydropyrimidine-5- |
| | carboxamide; |
| (42) | 4-(2-chlorophenyl)-2-((6- |
| | fluorobenzo[d]oxazol-2-yl)amino)-N-(5- |
| | (hydroxymethyl)pyridin-2-yl)-6-methyl-1,4- |
| | dihydropyrimidine-5-carboxamide; |
| (43) | 4-(2-chlorophenyl)-2-((6- |
| | fluorobenzo[d]oxazol-2-yl)amino)-6-methyl- |
| | N-(pyridin-2-yl)-1,4-dihydropyrimidine-5- |
| | carboxamide; |
| (44) | 4-(2-chlorophenyl)-2-((6- |
| | fluorobenzo[d]oxazol-2-yl)amino)-6-methyl- |
| | N-((1-methyl-1H-pyrazol-4-yl)methyl)-1,4- |
| | dihydropyrimidine-5-carboxamide; |
| (45) | 2-(4-(2-chloro-4-fluorophenyl)-2-((6- |
| | fluorobenzo[d]oxazol-2-yl)amino)-6-methyl- |
| | 1,4-dihydropyrimidine-5- |
| | carboxamido)isonicotinic acid; |
| (46) | 2-(4-(2-chloro-4-methoxyphenyl)-2-((6- |
| | fluorobenzo[d]oxazol-2-yl)amino)-6-methyl- |
| | 1,4-dihydropyrimidine-5- |
| | carboxamido)isonicotinic acid; |
| (47) | 2-(4-(2,4-dichlorophenyl)-2-((6- |
| | fluorobenzo[d]oxazol-2-yl)amino)-6-methyl- |
| | 1,4-dihydropyrimidine-5- |
| | carboxamido)isonicotinic acid; |
| (48) | 2-(4-(2-chloro-3-fluorophenyl)-2-((6- |
| | fluorobenzo[d]oxazol-2-yl)amino)-6-methyl- |
| | 1,4-dihydropyrimidine-5- |
| | carboxamido)isonicotinic acid; |
| (49) | 2-(4-(2,3-dichlorophenyl)-2-((6- |
| | fluorobenzo[d]oxazol-2-yl)amino)-6-methyl- |
| | 1,4-dihydropyrimidine-5- |
| | carboxamido)isonicotinic acid; |
| (50) | 2-(4-(2-chloro-4-methylphenyl)-2-((6- |
| | fluorobenzo[d]oxazol-2-yl)amino)-6-methyl- |
| | 1,4-dihydropyrimidine-5- |
| | carboxamido)isonicotinic acid; |
| (51) | 2-(4-(2-chloro-3-(trifluoromethyl)phenyl)-2- |
| | ((6-fluorobenzo[d]oxazol-2-yl)amino)-6- |
| | methyl-1,4-dihydropyrimidine-5- |
| | carboxamido)isonicotinic acid; |
| (52) | 2-(4-(2-chloro-3-methoxyphenyl)-2-((6- |
| | fluorobenzo[d]oxazol-2-yl)amino)-6-methyl- |
| | 1,4-dihydropyrimidine-5- |
| | carboxamido)isonicotinic acid; |
| (53) | (R)-2-(4-(2-chloro-4-methoxyphenyl)-2-((6- |
| | fluorobenzo[d]oxazol-2-yl)amino)-6-methyl- |
| | 1,4-dihydropyrimidine-5- |
| | carboxamido)isonicotinic acid; |
| (54) | (S)-2-(4-(2-chloro-4-methoxyphenyl)-2-((6- |
| | fluorobenzo[d]oxazol-2-yl)amino)-6-methyl- |
| | 1,4-dihydropyrimidine-5- |
| | carboxamido)isonicotinic acid; |
| (55) | (R)-2-(4-(2-chloro-4-methylphenyl)-2-((6- |
| | fluorobenzo[d]oxazol-2-yl)amino)-6-methyl- |
| | 1,4-dihydropyrimidine-5- |
| | carboxamido)isonicotinic acid; |
| (56) | (S)-2-(4-(2-chloro-4-methylphenyl)-2-((6- |
| | fluorobenzo[d]oxazol-2-yl)amino)-6-methyl- |
| | 1,4-dihydropyrimidine-5- |
| | carboxamido)isonicotinic acid; |
| (57) | 2-(4-(2-chloro-4-(pyrrolidin-1-yl)phenyl)-2- |
| | ((6-fluorobenzo[d]oxazol-2-yl)amino)-6- |
| | methyl-1,4-dihydropyrimidine-5- |
| | carboxamido)isonicotinic acid; |
| (58) | 2-(4-(2-chloro-4-(piperidin-1-yl)phenyl)-2-((6- |
| | fluorobenzo[d]oxazol-2-yl)amino)-6-methyl- |
| | 1,4-dihydropyrimidine-5- |
| | carboxamido)isonicotinic acid; |
| (59) | 2-(4-(2-chloro-4-morpholinphenyl)-2-((6- |
| | fluorobenzo[d]oxazol-2-yl)amino)-6-methyl- |
| | 1,4-dihydropyrimidine-5- |
| | carboxamido)isonicotinic acid; |
| (60) | 2-(4-(2-chloro-4-(1H-pyrazol-1-yl)phenyl)-2- |
| | ((6-fluorobenzo[d]oxazol-2-yl)amino)-6- |
| | methyl-1,4-dihydropyrimidine-5- |
| | carboxamido)isonicotinic acid; |
| (61) | 2-(4-(2-chloro-4-(trifluoromethyl)phenyl)-2- |
| | ((6-3.85fluorobenzo[d]oxazol-2-yl)amino)-6- |
| | methyl-1,4-dihydropyrimidine-5- |
| | carboxamido)isonicotinic acid; |
| (62) | 2-(4-(4-bromo-2-chlorophenyl)-2-((6- |
| | fluorobenzo[d]oxazol-2-yl)amino)-6-methyl- |
| | 1,4-dihydropyrimidine-5- |
| | carboxamido)isonicotinic acid; |
| (63) | 2-(4-(2-chloro-3-nitrophenyl)-2-((6- |
| | fluorobenzo[d]oxazol-2-yl)amino)-6-methyl- |
| | 1,4-dihydropyrimidine-5- |
| | carboxamido)isonicotinic acid; |
| (64) | 2-(4-(3-amino-2-chlorophenyl)-2-((6- |
| | fluorobenzo[d]oxazol-2-yl)amino)-6-methyl- |
| | 1,4-dihydropyrimidine-5- |
| | carboxamido)isonicotinic acid was; |
| (65) | 2-(4-(3-chloro-[1,1′-biphenyl]-4-yl)-2-((6- |
| | fluorobenzo[d]oxazol-2-yl)amino)-6-methyl- |
| | 1,4-dihydropyrimidine-5- |
| | carboxamido)isonicotinic acid; |
| (66) | 2-(4-(2-chloro-4-cyanophenyl)-2-((6- |
| | fluorobenzo[d]oxazol-2-yl)amino)-6-methyl- |
| | 1,4-dihydropyrimidine-5- |
| | carboxamido)isonicotinic acid; |
| (67) | 2-(4-(2-chloro-4-ethoxyphenyl)-2-((6- |
| | fluorobenzo[d]oxazol-2-yl)amino)-6-methyl- |
| | 1,4-dihydropyrimidine-5- |
| | carboxamido)isonicotinic acid; |
| (68) | 2-(4-(3-chloro-4′-(trifluoromethyl)-[1,1′- |
| | biphenyl]-4-yl)-2-((6-fluorobenzo[d]oxazol-2- |
| | yl)amino)-6-methyl-1,4-dihydropyrimidine-5- |
| | carboxamido)isonicotinic acid; |
| (69) | 2-(4-(2-chloro-4-(6-methylpyridin-3- |
| | yl)phenyl)-2-((6-fluorobenzo[d]oxazol-2- |
| | yl)amino)-6-methyl-1,4-dihydropyrimidine-5- |
| | carboxamido)isonicotinic acid; |
| (70) | 2-(4-(2-chloro-4-(6-(trifluoromethyl)pyridin-3- |
| | yl)phenyl)-2-((6-fluorobenzo[d]oxazol-2- |
| | yl)amino)-6-methyl-1,4-dihydropyrimidine-5- |
| | carboxamido)isonicotinic acid; |
| (71) | 2-(4-(2-chloro-4-(2-methylpyrimidin-5- |
| | yl)phenyl)-2-((6-fluorobenzo[d]oxazol-2- |
| | yl)amino)-6-methyl-1,4-dihydropyrimidine-5- |
| | carboxamido)isonicotinic acid; |
| (72) | 2-(4-(4-carboxy-2-chlorophenyl)-2-((6- |
| | fluorobenzo[d]oxazol-2-yl)amino)-6-methyl- |
| | 1,4-dihydropyrimidine-5- |
| | carboxamido)isonicotinic acid; |
| (73) | 2-(4-(5-carboxy-2-chlorophenyl)-2-((6- |
| | fluorobenzo[d]oxazol-2-yl)amino)-6-methyl- |
| | 1,4-dihydropyrimidine-5- |
| | carboxamido)isonicotinic acid; |
| (74) | 2-(4-(2-chloro-4-(1H-imidazol-1-yl)phenyl)-2- |
| | ((6-fluorobenzo[d]oxazol-2-yl)amino)-6- |
| | methyl-1,4-dihydropyrimidine-5- |
| | carboxamido)isonicotinic acid; |
| (75) | 2-(4-(2-bromophenyl)-2-((6- |
| | fluorobenzo[d]oxazol-2-yl)amino)-6-methyl- |
| | 1,4-dihydropyrimidine-5- |
| | carboxamido)isonicotinic acid; |
| (76) | 2-(4-(3-carbamoylphenyl)-2-((6- |
| | fluorobenzo[d]oxazol-2-yl)amino)-6-methyl- |
| | 1,4-dihydropyrimidine-5- |
| | carboxamido)isonicotinic acid; |
| (77) | 2-(2-((6-fluorobenzo[d]oxazol-2-yl)amino)-6- |
| | methyl-4-(3-(trifluoromethyl)phenyl)-1,4- |
| | dihydropyrimidine-5- |
| | carboxamido)isonicotinic acid; |
| (78) | 2-(2-((6-fluorobenzo[d]oxazol-2-yl)amino)-6- |
| | methyl-4-(3-sulfamoylphenyl)-1,4- |
| | dihydropyrimidine-5- |
| | carboxamido)isonicotinic acid; |
| (79) | 2-(2-((7-aminobenzo[d]oxazol-2-yl)amino)-4- |
| | (2-chloro-4-methylphenyl)-6-methyl-1,4- |
| | dihydropyrimidine-5- |
| | carboxamido)isonicotinic acid; |
| (80) | 2-(2-((7-aminobenzo[d]oxazol-2-yl)amino)-4- |
| | (2-chloro-4-methoxyphenyl)-6-methyl-1,4- |
| | dihydropyrimidine-5- |
| | carboxamido)isonicotinic acid; |
| (81) | 2-((7-aminobenzo[d]oxazol-2-yl)amino)-4-(2- |
| | chlorophenyl)-6-methyl-N-(pyridin-2-yl)-1,4- |
| | dihydropyrimidine-5-carboxamide; |
| (82) | 2-((7-aminobenzo[d]oxazol-2-yl)amino)-4-(2- |
| | chlorophenyl)-6-methyl-N-((1-methyl-1H- |
| | pyrazol-4-yl)methyl)-1,4-dihydropyrimidine- |
| | 5-carboxamide; |
| (83) | 2-((7-aminobenzo[d]oxazol-2-yl)amino)-4-(2- |
| | chloro-4-methylphenyl)-6-methyl-N-((1- |
| | methyl-1H-pyrazol-4-yl)methyl)-1,4- |
| | dihydropyrimidine-5-carboxamide; |
| (84) | (S)-2-(2-((7-aminobenzo[d]oxazol-2- |
| | yl)amino)-4-(2-chloro-4-methylphenyl)-6- |
| | methyl-1,4-dihydropyrimidine-5- |
| | carboxamido)isonicotinic acid; |
| (85) | (R)-2-(2-((7-aminobenzo[d]oxazol-2- |
| | yl)amino)-4-(2-chloro-4-methylphenyl)-6- |
| | methyl-1,4-dihydropyrimidine-5- |
| | carboxamido)isonicotinic acid; |
| (86) | (S)-2-(2-((7-aminobenzo[d]oxazol-2- |
| | yl)amino)-4-(2-chloro-4-methoxyphenyl)-6- |
| | methyl-1,4-dihydropyrimidine-5- |
| | carboxamido)isonicotinic acid; |
| (87) | (R)-2-(2-((7-aminobenzo[d]oxazol-2- |
| | yl)amino)-4-(2-chloro-4-methoxyphenyl)-6- |
| | methyl-1,4-dihydropyrimidine-5- |
| | carboxamido)isonicotinic acid; |
| (88) | N-(4-(2H-tetrazol-5-yl)pyridin-2-yl)-4-(2- |
| | chloro-4-methylphenyl)-2-((6- |
| | fluorobenzo[d]oxazol-2-yl)amino)-6-methyl- |
| | 1,4-dihydropyrimidine-5-carboxamide; |
| (89) | 2-((7-aminobenzo[d]oxazol-2-yl)amino)-4-(2- |
| | chloro-4-methylphenyl)-N-(4- |
| | (dimethylamino)pyridin-2-yl)-6-methyl-1,4- |
| | dihydropyrimidine-5-carboxamide; |
| (90) | 4-(2-chloro-4-methylphenyl)-N-(4- |
| | (dimethylamino)pyridin-2-yl)-2-((6- |
| | fluorobenzo[d]oxazol-2-yl)amino)-6-methyl- |
| | 1,4-dihydropyrimidine-5-carboxamide; |
| (91) | 2-(2-((7-aminobenzo[d]oxazol-2-yl)amino)-4- |
| | (2-chloro-4-methylphenyl)-6-methyl-1,4- |
| | dihydropyrimidine-5- |
| | carboxamido)isonicotinate; |
| (92) | (2-(4-(2-chloro-4-methylphenyl)-2-((6- |
| | fluorobenzo[d]oxazol-2-yl)amino)-6-methyl- |
| | 1,4-dihydropyrimidine-5- |
| | carboxamido)pyridin-4-yl)boronic acid; |
| (93) | 4-(2-chlorophenyl)-2-((6- |
| | methoxybenzo[d]oxazol-2-yl)amino)-6- |
| | methyl-N-(5-(pyridin-3-yl)-1,3,4-thiadiazol-2- |
| | yl)-1,4-dihydropyrimidine-5-carboxamide; |
| (94) | 2-((6-chlorobenzo[d]oxazol-2-yl)amino)-4-(2- |
| | chlorophenyl)-6-methyl-N-(5-(pyridin-3-yl)- |
| | 1,3,4-thiadiazol-2-yl)-1,4-dihydropyrimidine- |
| | 5-carboxamide; |
| (95) | 4-(2-chlorophenyl)-2-((5- |
| | methoxybenzo[d]oxazol-2-yl)amino)-6- |
| | methyl-N-(5-(pyridin-3-yl)-1,3,4-thiadiazol-2- |
| | yl)-1,4-dihydropyrimidine-5-carboxamide; |
| (96) | 4-(2-chlorophenyl)-2-((5- |
| | methylbenzo[d]oxazol-2-yl)amino)-6-methyl- |
| | N-(5-(pyridin-3-yl)-1,3,4-thiadiazol-2-yl)-1,4- |
| | dihydropyrimidine-5-carboxamide; |
| (97) | 4-(2-chlorophenyl)-2-((5- |
| | fluorobenzo[d]oxazol-2-yl)amino)-6-methyl- |
| | N-(5-(pyridin-3-yl)-1,3,4-thiadiazol-2-yl)-1,4- |
| | dihydropyrimidine-5-carboxamide; |
| (98) | 4-(2-chlorophenyl)-2-((5- |
| | chlorobenzo[d]oxazol-2-yl)amino)-6-methyl- |
| | N-(5-(pyridin-3-yl)-1,3,4-thiadiazol-2-yl)-1,4- |
| | dihydropyrimidine-5-carboxamide; |
| (99) | 4-(2-chlorophenyl)-2-((6- |
| | methylbenzo[d]oxazol-2-yl)amino)-6-methyl- |
| | N-(5-(pyridin-3-yl)-1,3,4-thiadiazol-2-yl)-1,4- |
| | dihydropyrimidine-5-carboxamide; |
| (100) | 4-(2-chlorophenyl)-2-((6- |
| | fluorobenzo[d]oxazol-2-yl)amino)-6-methyl- |
| | N-(5-(pyridin-3-yl)-1,3,4-thiadiazol-2-yl)-1,4- |
| | dihydropyrimidine-5-carboxamide; |
| (101) | 2-((5-aminobenzo[d]oxazol-2-yl)amino)- |
| | 4-(2-chlorophenyl)-6-methyl-N-(5-(pyridin-3- |
| | yl)-1,3,4-thiadiazol-2-yl)-1,4- |
| | dihydropyrimidine-5-carboxamide; |
| (102) | 2-(4-(2-chloro-4-methylphenyl)-6-methyl- |
| | 2-(oxazolo[4,5-b]pyridin-2-ylamino)-1,4- |
| | dihydropyrimidine-5- |
| | carboxamido)isonicotinic acid; |
| (103) | 2-(4-(2-chloro-4-methylphenyl)-6-methyl- |
| | 2-((5-phenyloxazol-2-yl)amino)-1,4- |
| | dihydropyrimidine-5- |
| | carboxamido)isonicotinic acid; |
| (104) | 4-(2-chloro-4-methylphenyl)-6-methyl-N- |
| | ((1-methyl-1H-pyrazol-4-yl)methyl)-2- |
| | (oxazolo[4,5-c]pyridin-2-ylamino)-1,4- |
| | dihydropyrimidine-5-carboxamide; |
| (105) | 4-(2-chloro-4-methylphenyl)-6-methyl-N- |
| | ((1-methyl-1H-pyrazol-4-yl)methyl)-2- |
| | (oxazolo[4,5-b]pyridin-2-ylamino)-1,4- |
| | dihydropyrimidine-5-carboxamide; |
| (106) | 2-(4-(2-chloro-4-methylphenyl)-6-methyl- |
| | 2-(oxazolo[4,5-c]pyridin-2-ylamino)-1,4- |
| | dihydropyrimidine-5- |
| | carboxamido)isonicotinic acid; |
| (107) | 4-(2-chloro-4-methylphenyl)-6-methyl-N- |
| | ((1-methyl-1H-pyrazol-4-yl)methyl)-2- |
| | (oxazolo[5,4-b]pyridin-2-ylamino)-1,4- |
| | dihydropyrimidine-5-carboxamide; |
| (108) | 2-(4-(2-chloro-4-methylphenyl)-6-methyl- |
| | 2-(oxazolo[5,4-b]pyridin-2-ylamino)-1,4- |
| | dihydropyrimidine-5- |
| | carboxamido)isonicotinic acid; |
| (109) | 2-(4-(2-chloro-4-methylphenyl)-6-methyl- |
| | 2-((4,5,6,7-tetrahydrobenzo[d]oxazol-2- |
| | yl)amino)-1,4-dihydropyrimidine-5- |
| | carboxamido)isonicotinic acid; |
| (110) | N-((1-benzyl-1H-pyrazol-4-yl)methyl)-4- |
| | (2-chloro-4-methylphenyl)-2-((6- |
| | fluorobenzo[d]oxazol-2-yl)amino)-6-methyl- |
| | 1,4-dihydropyrimidine-5-carboxamide; |
| (111) | 4-(2-chloro-4-methylphenyl)-2-((6- |
| | fluorobenzo[d]oxazol-2-yl)amino)-6-methyl- |
| | N-((1,3,5-trimethyl-1H-pyrazol-4-yl)methyl)- |
| | 1,4-dihydropyrimidine-5-carboxamide; |
| (112) | 4-(2-chloro-4-methylphenyl)-N-((1-ethyl- |
| | 1H-pyrazol-4-yl)methyl)-2-((6- |
| | fluorobenzo[d]oxazol-2-yl)amino)-6-methyl- |
| | 1,4-dihydropyrimidine-5-carboxamide; |
| (113) | 2-((7-aminobenzo[d]oxazol-2-yl)amino)- |
| | 4-(2-chloro-4-methylphenyl)-6-methyl-N- |
| | ((1,3,5-trimethyl-1H-pyrazol-4-yl)methyl)-1,4- |
| | dihydropyrimidine-5-carboxamide; |
| (114) | 2-((7-aminobenzo[d]oxazol-2-yl)amino)- |
| | N-((1-benzyl-1H-pyrazol-4-yl)methyl)-4-(2- |
| | chloro-4-methylphenyl)-6-methyl-1,4- |
| | dihydropyrimidine-5-carboxamide; |
| (115) | (R)-2-((7-aminobenzo[d]oxazol-2- |
| | yl)amino)-4-(2-chloro-4-methylphenyl)-6- |
| | methyl-N-((1-methyl-1H-pyrazol-4- |
| | yl)methyl)-1,4-dihydropyrimidine-5- |
| | carboxamide; |
| (116) | (S)-2-((7-aminobenzo[d]oxazol-2- |
| | yl)amino)-4-(2-chloro-4-methylphenyl)-6- |
| | methyl-N-((1-methyl-1H-pyrazol-4- |
| | yl)methyl)-1,4-dihydropyrimidine-5- |
| | carboxamide; |
| (117) | 2-((7-aminobenzo[d]oxazol-2-yl)amino)- |
| | 4-(2-chloro-4-methylphenyl)-6-methyl-N-((1- |
| | ethyl-1H-pyrazol-4-yl)methyl)-1,4- |
| | dihydropyrimidine-5-carboxamide; |
| (118) | 2-((7-aminobenzo[d]oxazol-2-yl)amino)- |
| | 4-(2-chloro-4-fluorophenyl)-N-((1-ethyl-1H- |
| | pyrazol-4-yl)methyl)-6-methyl-1,4- |
| | dihydropyrimidine-5-carboxamide; |
| (119) | 2-((7-aminobenzo[d]oxazol-2-yl)amino)- |
| | 4-(2-chloro-4-fluorophenyl)-N-((1-methyl-1H- |
| | pyrazol-4-yl)methyl)-6-methyl-1,4- |
| | dihydropyrimidine-5-carboxamide; |
| (120) | 2-((7-aminobenzo[d]oxazol-2-yl)amino)- |
| | 4-(2,4-dichlorophenyl)-N-((1-ethyl-1H- |
| | pyrazol-4-yl)methyl)-6-methyl-1,4- |
| | dihydropyrimidine-5-carboxamide; |
| (121) | 2-((7-aminobenzo[d]oxazol-2-yl)amino)- |
| | 4-(2-chloro-4-methylphenyl)-6-methyl-N-((1- |
| | ethyl-1H-pyrazol-4-yl)methyl)-1,4- |
| | dihydropyrimidine-5-carboxamide; |
| (122) | 2-((7-aminobenzo[d]oxazol-2-yl)amino)- |
| | 4-(2-chloro-4-methylphenyl)-6-methyl-N-((1- |
| | ethyl-1H-pyrazol-4-yl)methyl)-1,4- |
| | dihydropyrimidine-5-carboxamide; |
| (123) | 2-((7-aminobenzo[d]oxazol-2-yl)amino)- |
| | 4-(2,4-dichlorophenyl)-N-((1-methyl-1H- |
| | pyrazol-4-yl)methyl)-6-methyl-1,4- |
| | dihydropyrimidine-5-carboxamide; |
| (124) | 4-(2-chloro-4-methylphenyl)-N-((1,5- |
| | dimethyl-1H-pyrazol-4-yl)methyl)-2-((6- |
| | fluorobenzo[d]oxazol-2-yl)amino)-6-methyl- |
| | 1,4-dihydropyrimidine-5-carboxamide; |
| (125) | 2-((7-amino-6-fluorobenzo[d]oxazol-2- |
| | yl)amino)-4-(2-chloro-4-methylphenyl)-N-((1- |
| | ethyl-1H-pyrazol-4-yl)methyl)-6-methyl-1,4- |
| | dihydropyrimidine-5-carboxamide; |
| (126) | 2-((7-amino-6-fluorobenzo[d]oxazol-2- |
| | yl)amino)-4-(2-chloro-4-methylphenyl)-6- |
| | methyl-N-((1-methyl-1H-pyrazol-4- |
| | yl)methyl)-1,4-dihydropyrimidine-5- |
| | carboxamide; |
| (127) | 2-((7-amino-6-fluorobenzo[d]oxazol-2- |
| | yl)amino)-4-(2,4-dichlorophenyl)-6-methyl-N- |
| | ((1-methyl-1H-pyrazol-4-yl)methyl)-1,4- |
| | dihydropyrimidine-5-carboxamide; |
| (128) | 2-((4-(2-chloro-4-methylphenyl)-6- |
| | methyl-5-(((1-methyl-1H-pyrazol-4- |
| | yl)methyl)carbamoyl)-1,4-dihydropyrimidin- |
| | 2-yl)amino)-6-fluorobenzo[d]oxazole-7- |
| | carboxylic acid; |
| (129) | 2-((7-amino-6-fluorobenzo[d]oxazol-2- |
| | yl)amino)-4-(2-chloro-4-methylphenyl)-N-((1- |
| | ethyl-5-fluoro-3-methyl-1H-pyrazol-4- |
| | yl)methyl)-6-methyl-1,4-dihydropyrimidine-5- |
| | carboxamide; |
| (130) | 2-((7-amino-6-fluorobenzo[d]oxazol-2- |
| | yl)amino)-4-(2,4-dichlorophenyl)-N-((1-ethyl- |
| | 1H-pyrazol-4-yl)methyl)-6-methyl-1,4- |
| | dihydropyrimidine-5-carboxamide; |
| (131) | 2-((7-amino-6-fluorobenzo[d]oxazol-2- |
| | yl)amino)-4-(2-chloro-4-methylphenyl)-6- |
| | methyl-N-((1-(2,2,2-trifluoroethyl)-1H- |
| | pyrazol-4-yl)methyl)-1,4-dihydropyrimidine- |
| | 5-carboxamide; |
| (132) | 2-((7-amino-6-fluorobenzo[d]oxazol-2- |
| | yl)amino)-N-((1-(tert-butyl)-1H-pyrazol-4- |
| | yl)methyl)-4-(2-chloro-4-methylphenyl)-6- |
| | methyl-1,4-dihydropyrimidine-5- |
| | carboxamide; |
| (133) | 2-((7-amino-6-fluorobenzo[d]oxazol-2- |
| | yl)amino)-4-(2-chloro-4-methylphenyl)-N-((1- |
| | ethyl-5-fluoro-1H-pyrazol-4-yl)methyl)-6- |
| | methyl-1,4-dihydropyrimidine-5- |
| | carboxamide; |
| (134) | (S)-2-((7-amino-6-fluorobenzo[d]oxazol- |
| | 2-yl)amino)-4-(2-chloro-4-methylphenyl)-N- |
| | ((1-ethyl-1H-pyrazol-4-yl)methyl)-6-methyl- |
| | 1,4-dihydropyrimidine-5-carboxamide; |
| (135) | (R)-2-((7-amino-6-fluorobenzo[d]oxazol- |
| | 2-yl)amino)-4-(2-chloro-4-methylphenyl)-N- |
| | ((1-ethyl-1H-pyrazol-4-yl)methyl)-6- |
| | 1,4-dihydropyrimidine-5-carboxamide; |
| (136) | 2-((7-amino-6-fluorobenzo[d]oxazol-2- |
| | yl)amino)-4-(2-chlorophenyl)-N-((1-ethyl-1H- |
| | methyl-pyrazol-4-yl)methyl)-6-methyl-1,4- |
| | dihydropyrimidine-5-carboxamide; |
| (137) | 2-(benzo[d]oxazol-2-ylamino)-4-(2- |
| | chlorophenyl)-6-methyl-N-phenyl-1,4- |
| | dihydropyrimidine-5-carboxamide; |
| (138) | 4-(2-(benzo[d]oxazol-2-ylamino)-4-(2- |
| | chlorophenyl)-6-methyl-1,4- |
| | dihydropyrimidine-5-carboxamido)benzoate; |
| (139) | ethyl 3-(2-(benzo[d]oxazol-2-ylamino)-4- |
| | (2-chlorophenyl)-6-methyl-1,4- |
| | dihydropyrimidine-5-carboxamido)benzoate; |
| (140) | 2-(benzo[d]oxazol-2-ylamino)-N,4-bis(2- |
| | chlorophenyl)-6-methyl-1,4- |
| | dihydropyrimidine-5-carboxamide; |
| (141) | 2-(benzo[d]oxazol-2-ylamino)-4-(2- |
| | chlorophenyl)-N-(4-chlorophenyl)-6-methyl- |
| | 1,4-dihydropyrimidine-5-carboxamide; |
| (142) | 3-(2-(benzo[d]oxazol-2-ylamino)-4-(2- |
| | chlorophenyl)-6-methyl-1,4- |
| | dihydropyrimidine-5-carboxamido)benzoic |
| | acid; |
| (143) | 4-(2-(benzo[d]oxazol-2-ylamino)-4-(2- |
| | chlorophenyl)-6-methyl-1,4- |
| | dihydropyrimidine-5-carboxamido)benzoic |
| | acid; |
| (144) | 2-(benzo[d]oxazol-2-ylamino)-4-(2- |
| | chlorophenyl)-N-(3-(hydroxymethyl)phenyl)- |
| | 6-methyl-1,4-dihydropyrimidine-5- |
| | carboxamide; |
| (145) | (2-(benzo[d]oxazol-2-ylamino)-4-(2- |
| | chlorophenyl)-6-methyl-1,4- |
| | dihydropyrimidin-5- |
| | yl)(morpholino)methanone; |
| (146) | 2-(2-(benzo[d]oxazol-2-ylamino)-4-(2- |
| | chlorophenyl)-6-methyl-1,4- |
| | dihydropyrimidine-5-carboxamido)benzoate; |
| (147) | 2-(benzo[d]oxazol-2-ylamino)-N-(3- |
| | bromophenyl)-4-(2-chlorophenyl)-6-methyl- |
| | 1,4-dihydropyrimidine-5-carboxamide; |
| (148) | 2-(benzo[d]oxazol-2-ylamino)-N-(4- |
| | fluorophenyl)-4-(2-chlorophenyl)-6-methyl- |
| | 1,4-dihydropyrimidine-5-carboxamide; |
| (149) | 2-(benzo[d]oxazol-2-ylamino)-N-(pyridin- |
| | 3-yl)-4-(2-chlorophenyl)-6-methyl-1,4- |
| | dihydropyrimidine-5-carboxamide; |
| (150) | 2-(benzo[d]oxazol-2-ylamino)-N-(4- |
| | bromophenyl)-4-(2-chlorophenyl)-6-methyl- |
| | 1,4-dihydropyrimidine-5-carboxamide; |
| (151) | 2-(benzo[d]oxazol-2-ylamino)-4-(2- |
| | chlorophenyl)-N,6-dimethyl-N-phenyl-1,4- |
| | dihydropyrimidine-5-carboxamide; |
| (152) | 2-(benzo[d]oxazol-2-ylamino)-4-(2- |
| | chlorophenyl)-N-(4-(dimethylamino)phenyl)- |
| | 6-methyl-1,4-dihydropyrimidine-5- |
| | carboxamide; |
| (153) | 2-(benzo[d]oxazol-2-ylamino)-4-(2- |
| | chlorophenyl)-N-(5-chloropyridin-2-yl)-6- |
| | methyl-1,4-dihydropyrimidine-5- |
| | carboxamide; |
| (154) | 2-(benzo[d]oxazol-2-ylamino)-N-(3- |
| | chlorophenyl)-4-(2-chlorophenyl)-6-methyl- |
| | 1,4-dihydropyrimidine-5-carboxamide; |
| (155) | 2-(benzo[d]oxazol-2-ylamino)-4-(2- |
| | chlorophenyl)-6-methyl-N-(2- |
| | methylbenzo[d]thiazol-6-yl)-1,4- |
| | dihydropyrimidine-5-carboxamide; |
| (156) | 2-(benzo[d]oxazol-2-ylamino)-4-(2- |
| | chlorophenyl)-N-(2-(hydroxymethyl)phenyl)- |
| | 6-methyl-1,4-dihydropyrimidine-5- |
| | carboxamide; |
| (157) | 2-(benzo[d]oxazol-2-ylamino)-N-(2- |
| | fluorophenyl)-4-(2-chlorophenyl)-6-methyl- |
| | 1,4-dihydropyrimidine-5-carboxamide; |
| (158) | 2-(benzo[d]oxazol-2-ylamino)-N-(3- |
| | fluorophenyl)-4-(2-chlorophenyl)-6-methyl- |
| | 1,4-dihydropyrimidine-5-carboxamide; |
| (159) | 2-(2-(benzo[d]oxazol-2-ylamino)-4-(2- |
| | chlorophenyl)-6-methyl-1,4- |
| | dihydropyrimidine-5-carboxamido)benzoic |
| | acid; |
| (160) | 2-(benzo[d]oxazol-2-ylamino)-4-(2- |
| | chlorophenyl)-N-(4-(hydroxymethyl)phenyl)- |
| | 6-methyl-1,4-dihydropyrimidine-5- |
| | carboxamide; |
| (161) | 2-(benzo[d]oxazol-2-ylamino)-N-(2- |
| | hydroxyphenyl)-4-(2-chlorophenyl)-6-methyl- |
| | 1,4-dihydropyrimidine-5-carboxamide; |
| (162) | 2-(benzo[d]oxazol-2-ylamino)-N-(4- |
| | hydroxyphenyl)-4-(2-chlorophenyl)-6-methyl- |
| | 1,4-dihydropyrimidine-5-carboxamide; |
| (163) | 2-(benzo[d]oxazol-2-ylamino)-4-(2- |
| | chlorophenyl)-6-methyl-N-(4-nitrophenyl)- |
| | 1,4-dihydropyrimidine-5-carboxamide; |
| (164) | 5-(2-(benzo[d]oxazol-2-ylamino)-4-(2- |
| | chlorophenyl)-6-methyl-1,4- |
| | dihydropyrimidine-5-carboxamido)picolinic |
| | acid; |
| (165) | 2-(benzo[d]oxazol-2-ylamino)-4-(2- |
| | chlorophenyl)-N-(6-(hydroxymethyl)pyridin- |
| | 3-yl)-6-methyl-1,4-dihydropyrimidine-5- |
| | carboxamide; |
| (166) | methyl 5-(2-(benzo[d]oxazol-2-ylamino)- |
| | 4-(2-chlorophenyl)-6-methyl-1,4- |
| | dihydropyrimidine-5-carboxamido)picolinate; |
| (167) | 2-(benzo[d]oxazol-2-ylamino)-4-(2- |
| | chlorophenyl)-6-methyl-N-(5- |
| | (trifluoromethyl)-1,3,4-thiadiazol-2-yl)-1,4- |
| | dihydropyrimidine-5-carboxamide; |
| (168) | ethyl 5-(2-(benzo[d]oxazol-2-ylamino)-4- |
| | (2-chlorophenyl)-6-methyl-1,4- |
| | dihydropyrimidine-5-carboxamido)-1,3,4- |
| | thiadiazole-2-carboxylate; |
| (169) | N-(4-(aminomethyl)phenyl)-2- |
| | (benzo[d]oxazol-2-ylamino)-4-(2- |
| | chlorophenyl)-6-methyl-1,4- |
| | dihydropyrimidine-5-carboxamide; |
| (170) | methyl 2-(4-(2-(benzo[d]oxazol-2- |
| | ylamino)-4-(2-chlorophenyl)-6-methyl-1,4- |
| | dihydropyrimidine-5- |
| | carboxamido)phenyl)acetate; |
| (171) | N-(3-(aminomethyl)phenyl)-2- |
| | (benzo[d]oxazol-2-ylamino)-4-(2- |
| | chlorophenyl)-6-methyl-1,4- |
| | dihydropyrimidine-5-carboxamide; |
| (172) | N-(3-(1H-pyrazol-3-yl)phenyl)-2- |
| | (benzo[d]oxazol-2-ylamino)-4-(2- |
| | chlorophenyl)-6-methyl-1,4- |
| | dihydropyrimidine-5-carboxamide; |
| (173) | 2-(benzo[d]oxazol-2-ylamino)-4-(2- |
| | chlorophenyl)-6-methyl-N-(5-methyl-1,3,4- |
| | thiadiazol-2-yl)-1,4-dihydropyrimidine-5- |
| | carboxamide; |
| (174) | methyl 2-(5-(2-(benzo[d]oxazol-2- |
| | ylamino)-4-(2-chlorophenyl)-6-methyl-1,4- |
| | dihydropyrimidine-5-carboxamido)-1,3,4- |
| | thiadiazol-2-yl)acetate; |
| (175) | N-(3-(1H-tetrazol-5-yl)phenyl)-2- |
| | (benzo[d]oxazol-2-ylamino)-4-(2- |
| | chlorophenyl)-6-methyl-1,4- |
| | dihydropyrimidine-5-carboxamide; |
| (176) | methyl 2-(3-(2-(benzo[d]oxazol-2- |
| | ylamino)-4-(2-chlorophenyl)-6-methyl-1,4- |
| | dihydropyrimidine-5- |
| | carboxamido)phenyl)acetate; |
| (177) | 2-(benzo[d]oxazol-2-ylamino)-N-(3- |
| | carbamoylphenyl)-4-(2-chlorophenyl)-6- |
| | methyl-1,4-dihydropyrimidine-5- |
| | carboxamide; |
| (178) | N-(3-(1H-pyrazol-4-yl)phenyl)-2- |
| | (benzo[d]oxazol-2-ylamino)-4-(2- |
| | chlorophenyl)-6-methyl-1,4- |
| | dihydropyrimidine-5-carboxamide; |
| (179) | N-(4-(1H-pyrazol-4-yl)phenyl)-2- |
| | (benzo[d]oxazol-2-ylamino)-4-(2- |
| | chlorophenyl)-6-methyl-1,4- |
| | dihydropyrimidine-5-carboxamide; |
| (180) | 2-(benzo[d]oxazol-2-ylamino)-4-(2- |
| | chlorophenyl)-6-methyl-N-(5-(pyridin-3-yl)- |
| | 1,3,4-thiadiazol-2-yl)-1,4-dihydropyrimidine- |
| | 5-carboxamide; |
| (181) | 2-(benzo[d]oxazol-2-ylamino)-N-(4- |
| | carbamoylphenyl)-4-(2-chlorophenyl)-6- |
| | methyl-1,4-dihydropyrimidine-5- |
| | carboxamide; |
| (182) | 2-(benzo[d]oxazol-2-ylamino)-4-(2- |
| | chlorophenyl)-6-methyl-N-(4-(morpholine-4- |
| | carbonyl)phenyl)-1,4-dihydropyrimidine-5- |
| | carboxamide; |
| (183) | 2-(benzo[d]oxazol-2-ylamino)-4-(2- |
| | chlorophenyl)-6-methyl-N-(4- |
| | (morpholinomethyl)phenyl)-1,4- |
| | dihydropyrimidine-5-carboxamide; |
| (184) | N-(4-(1H-pyrazol-1-yl)phenyl)-2- |
| | (benzo[d]oxazol-2-ylamino)-4-(2- |
| | chlorophenyl)-6-methyl-1,4- |
| | dihydropyrimidine-5-carboxamide; |
| (185) | methyl 2-(2-(benzo[d]oxazol-2-ylamino)- |
| | 4-(2-chlorophenyl)-6-methyl-1,4- |
| | dihydropyrimidine-5-carboxamido)thiazole-5- |
| | carboxylate; |
| (186) | methyl 2-(2-(benzo[d]oxazol-2-ylamino)- |
| | 4-(2-chlorophenyl)-6-methyl-1,4- |
| | dihydropyrimidine-5-carboxamido)thiazole- |
| | 4-carboxylate; |
| (187) | 2-(benzo[d]oxazol-2-ylamino)-4-(2- |
| | chlorophenyl)-6-methyl-N-(5-(pyridin-4-yl)- |
| | 1,3,4-thiadiazol-2-yl)-1,4-dihydropyrimidine- |
| | 5-carboxamide; |
| (188) | methyl 5-(2-(benzo[d]oxazol-2-ylamino)- |
| | 4-(2-chlorophenyl)-6-methyl-1,4- |
| | dihydropyrimidine-5-carboxamido)nicotinate; |
| (189) | 2-(benzo[d]oxazol-2-ylamino)-4-(2- |
| | chlorophenyl)-N-(4-cyanophenyl)-6-methyl- |
| | 1,4-dihydropyrimidine-5-carboxamide; |
| (190) | 2-(benzo[d]oxazol-2-ylamino)-4-(2- |
| | chlorophenyl)-6-methyl-N-(4-(piperazin-1- |
| | yl)phenyl)-1,4-dihydropyrimidine-5- |
| | carboxamide; |
| (191) | N-(4-(1H-1,2,4-triazol-1-yl)phenyl)-2- |
| | (benzo[d]oxazol-2-ylamino)-4-(2- |
| | chlorophenyl)-6-methyl-1,4- |
| | dihydropyrimidine-5-carboxamide; |
| (192) | N-(4-(1H-tetrazol-5-yl)phenyl)-2- |
| | (benzo[d]oxazol-2-ylamino)-4-(2- |
| | chlorophenyl)-6-methyl-1,4- |
| | dihydropyrimidine-5-carboxamide; |
| (193) | 2-(benzo[d]oxazol-2-ylamino)-4-(2- |
| | chlorophenyl)-N-(5-(hydroxymethyl)thiazol- |
| | 2-yl)-6-methyl-1,4-dihydropyrimidine-5- |
| | carboxamide; |
| (194) | 2-(2-(benzo[d]oxazol-2-ylamino)-4-(2- |
| | chlorophenyl)-6-methyl-1,4- |
| | dihydropyrimidine-5- |
| | carboxamido)isonicotinic acid; |
| (195) | 6-(2-(benzo[d]oxazol-2-ylamino)-4-(2- |
| | chlorophenyl)-6-methyl-1,4- |
| | dihydropyrimidine-5-carboxamido)picolinic |
| | acid; |
| (196) | methyl 2-(2-(benzo[d]oxazol-2-ylamino)- |
| | 4-(2-chlorophenyl)-6-methyl-1,4- |
| | dihydropyrimidine-5- |
| | carboxamido)isonicotinate; |
| (197) | methyl 2-(2-(benzo[d]oxazol-2-ylamino)- |
| | 4-(2-chlorophenyl)-6-methyl-1,4- |
| | dihydropyrimidine-5- |
| | carboxamido)isonicotinate; |
| (198) | 2-(benzo[d]oxazol-2-ylamino)-4-(2- |
| | chlorophenyl)-N-(4-(hydroxymethyl)thiazol- |
| | 2-yl)-6-methyl-1,4-dihydropyrimidine-5- |
| | carboxamide; |
| (199) | 2-(2-(benzo[d]oxazol-2-ylamino)-4-(2- |
| | chlorophenyl)-6-methyl-1,4- |
| | dihydropyrimidine-5-carboxamido)thiazole-5- |
| | carboxylic acid; |
| (200) | 2-(2-(benzo[d]oxazol-2-ylamino)-4-(2- |
| | chlorophenyl)-6-methyl-1,4- |
| | dihydropyrimidine-5-carboxamido)thiazole- |
| | 4-carboxylic acid; |
| (201) | 2-(benzo[d]oxazol-2-ylamino)-4-(2- |
| | chlorophenyl)-N-(3-(2-hydroxyethyl)phenyl)- |
| | 6-methyl-1,4-dihydropyrimidine-5- |
| | carboxamide; |
| (202) | 5-(2-(benzo[d]oxazol-2-ylamino)-4-(2- |
| | chlorophenyl)-6-methyl-1,4- |
| | dihydropyrimidine-5-carboxamido)nicotinic |
| | acid; |
| (203) | 4-(2-(benzo[d]oxazol-2-ylamino)-4-(2- |
| | chlorophenyl)-6-methyl-1,4- |
| | dihydropyrimidine-5-carboxamido)picolinic |
| | acid; |
| (204) | methyl 4-(2-(benzo[d]oxazol-2-ylamino)- |
| | 4-(2-chlorophenyl)-6-methyl-1,4- |
| | dihydropyrimidine-5-carboxamido)picolinate; |
| (205) | 2-(benzo[d]oxazol-2-ylamino)-4-(2- |
| | chlorophenyl)-6-methyl-N-(1,3,4-thiadiazol- |
| | 2-yl)-1,4-dihydropyrimidine-5-carboxamide; |
| (206) | (R)-methyl 2-(2-(benzo[d]oxazol-2- |
| | ylamino)-4-(2-chlorophenyl)-6-methyl-1,4- |
| | dihydropyrimidine-5- |
| | carboxamido)isonicotinate; |
| (207) | (S)-methyl 2-(2-(benzo[d]oxazol-2- |
| | ylamino)-4-(2-chlorophenyl)-6-methyl-1,4- |
| | dihydropyrimidine-5- |
| | carboxamido)isonicotinate; |
| (208) | (R)-2-(2-(benzo[d]oxazol-2-ylamino)-4- |
| | (2-chlorophenyl)-6-methyl-1,4- |
| | dihydropyrimidine-5- |
| | carboxamido)isonicotinic acid; |
| (209) | (S)-2-(2-(benzo[d]oxazol-2-ylamino)-4-(2- |
| | chlorophenyl)-6-methyl-1,4- |
| | dihydropyrimidine-5- |
| | carboxamido)isonicotinic acid; |
| (210) | 3-(2-(benzo[d]oxazol-2-ylamino)-4-(2- |
| | chlorophenyl)-6-methyl-1,4- |
| | dihydropyrimidine-5-carboxamido)-4- |
| | fluorobenzoic acid; |
| (211) | 2-(benzo[d]oxazol-2-ylamino)-4-(2- |
| | chlorophenyl)-N-(4-(hydroxymethyl)pyridin- |
| | 2-yl)-6-methyl-1,4-dihydropyrimidine-5- |
| | carboxamide; |
| (212) | 2-(benzo[d]oxazol-2-ylamino)-N-(4- |
| | carbamoylpyridin-2-yl)-4-(2-chlorophenyl)-6- |
| | methyl-1,4-dihydropyrimidine-5- |
| | carboxamide; |
| (213) | 2-(benzo[d]oxazol-2-ylamino)-4-(2- |
| | chlorophenyl)-6-methyl-N-(4- |
| | (methylcarbamoyl)pyridin-2-yl)-1,4- |
| | dihydropyrimidine-5-carboxamide; |
| (214) | 2-(benzo[d]oxazol-2-ylamino)-4-(2- |
| | chlorophenyl)-6-methyl-N-(pyridin-2-yl)-1,4- |
| | dihydropyrimidine-5-carboxamide; |
| (215) | 2-(benzo[d]oxazol-2-ylamino)-4-(2- |
| | chlorophenyl)-N-(2-methoxybenzyl)-6- |
| | methyl-1,4-dihydropyrimidine-5- |
| | carboxamide; |
| (216) | 2-(benzo[d]oxazol-2-ylamino)-N-(2- |
| | chlorobenzyl)-4-(2-chlorophenyl)-6-methyl- |
| | 1,4-dihydropyrimidine-5-carboxamide; |
| (217) | 2-(benzo[d]oxazol-2-ylamino)-4-(2- |
| | chlorophenyl)-N-(4-fluorobenzyl)-6-methyl- |
| | 1,4-dihydropyrimidine-5-carboxamide; |
| (218) | 2-(benzo[d]oxazol-2-ylamino)-4-(2- |
| | chlorophenyl)-N-(4-chlorobenzyl)-6-methyl- |
| | 1,4-dihydropyrimidine-5-carboxamide; |
| (219) | 2-(benzo[d]oxazol-2-ylamino)-4-(2- |
| | chlorophenyl)-N-(2-hydroxybenzyl)-6- |
| | methyl-1,4-dihydropyrimidine-5- |
| | carboxamide; |
| (220) | 2-(benzo[d]oxazol-2-ylamino)-4-(2- |
| | chlorophenyl)-6-methyl-N-(pyridin-2- |
| | ylmethyl)-1,4-dihydropyrimidine-5- |
| | carboxamide; |
| (221) | 2-(benzo[d]oxazol-2-ylamino)-4-(2- |
| | chlorophenyl)-6-methyl-N-(pyridin-3- |
| | ylmethyl)-1,4-dihydropyrimidine-5- |
| | carboxamide; |
| (222) | methyl 2-((2-(benzo[d]oxazol-2-ylamino)- |
| | 4-(2-chlorophenyl)-6-methyl-1,4- |
| | dihydropyrimidine-5- |
| | carboxamido)methyl)benzoate; |
| (223) | methyl 4-((2-(benzo[d]oxazol-2-ylamino)- |
| | 4-(2-chlorophenyl)-6-methyl-1,4- |
| | dihydropyrimidine-5- |
| | carboxamido)methyl)benzoate; |
| (224) | methyl 3-((2-(benzo[d]oxazol-2-ylamino)- |
| | 4-(2-chlorophenyl)-6-methyl-1,4- |
| | dihydropyrimidine-5- |
| | carboxamido)methyl)benzoate; |
| (225) | 4-((2-(benzo[d]oxazol-2-ylamino)-4-(2- |
| | chlorophenyl)-6-methyl-1,4- |
| | dihydropyrimidine-5- |
| | carboxamido)methyl)benzoic acid; |
| (226) | 3-((2-(benzo[d]oxazol-2-ylamino)-4-(2- |
| | chlorophenyl)-6-methyl-1,4- |
| | dihydropyrimidine-5- |
| | carboxamido)methyl)benzoic acid; |
| (227) | 2-(benzo[d]oxazol-2-ylamino)-4-(2- |
| | chlorophenyl)-N-(3-(hydroxymethyl)benzyl)- |
| | 6-methyl-1,4-dihydropyrimidine-5- |
| | carboxamide; |
| (228) | 2-(benzo[d]oxazol-2-ylamino)-4-(2- |
| | chlorophenyl)-N-(4-(hydroxymethyl)benzyl)- |
| | 6-methyl-1,4-dihydropyrimidine-5- |
| | carboxamide; |
| (229) | 2-(benzo[d]oxazol-2-ylamino)-4-(2- |
| | chlorophenyl)-N-(2-(hydroxymethyl)benzyl)- |
| | 6-methyl-1,4-dihydropyrimidine-5- |
| | carboxamide; |
| (230) | 2-(benzo[d]oxazol-2-ylamino)-4-(2- |
| | chlorophenyl)-6-methyl-N-((1-methyl-1H- |
| | imidazol-5-yl)methyl)-1,4-dihydropyrimidine- |
| | 5-carboxamide; |
| (231) | 2-(benzo[d]oxazol-2-ylamino)-4-(2- |
| | chlorophenyl)-6-methyl-N-((1-methyl-1H- |
| | imidazol-2-yl)methyl)-1,4-dihydropyrimidine- |
| | 5-carboxamide; |
| (232) | 2-(benzo[d]oxazol-2-ylamino)-4-(2- |
| | chlorophenyl)-6-methyl-N-(thiazol-5- |
| | ylmethyl)-1,4-dihydropyrimidine-5- |
| | carboxamide; |
| (233) | 2-(benzo[d]oxazol-2-ylamino)-4-(2- |
| | chlorophenyl)-6-methyl-N-((1-methyl-1H- |
| | imidazol-4-yl)methyl)-1,4-dihydropyrimidine- |
| | 5-carboxamide; |
| (234) | 2-(benzo[d]oxazol-2-ylamino)-4-(2- |
| | chlorophenyl)-6-methyl-N-((1-methyl-1H- |
| | benzo[d]imidazol-2-yl)methyl)-1,4- |
| | dihydropyrimidine-5-carboxamide; |
| (235) | (R)-2-(benzo[d]oxazol-2-ylamino)-4-(2- |
| | chlorophenyl)-6-methyl-N-((1-methyl-1H- |
| | imidazol-4-yl)methyl)-1,4-dihydropyrimidine- |
| | 5-carboxamide; |
| (236) | (S)-2-(benzo[d]oxazol-2-ylamino)-4-(2- |
| | chlorophenyl)-6-methyl-N-((1-methyl-1H- |
| | imidazol-4-yl)methyl)-1,4-dihydropyrimidine- |
| | 5-carboxamide; |
| (237) | 2-(benzo[d]oxazol-2-ylamino)-4-(2- |
| | chlorophenyl)-N-(2-(dimethylamino)ethyl)-6- |
| | methyl-1,4-dihydropyrimidine-5- |
| | carboxamide; |
| (238) | 2-(benzo[d]oxazol-2-ylamino)-4-(2- |
| | chlorophenyl)-6-methyl-N-((1-methyl-1H- |
| | pyrazol-4-yl)methyl)-1,4-dihydropyrimidine- |
| | 5-carboxamide; |
| (239) | ethyl 2-(benzo[d]oxazol-2-ylamino)-4-(2- |
| | chlorophenyl)-6-methyl-1,4- |
| | dihydropyrimidine-5-carboxylate; |
| (240) | ethyl 2-(benzo[d]oxazol-2-ylamino)-4-(2- |
| | chlorophenyl)-6-isopropyl-1,4- |
| | dihydropyrimidine-5-carboxylate; |
| (241) | methyl 2-(benzo[d]oxazol-2-ylamino)-4- |
| | (2-chlorophenyl)-6-(trifluoromethyl)-1,4- |
| | dihydropyrimidine-5-carboxylate; |
| (242) | dimethyl (2-(benzo[d]oxazol-2-ylamino)- |
| | 4-(2-chlorophenyl)-6-methyl-1,4- |
| | dihydropyrimidin-5-yl)phosphonate; |
| (243) | N-(4-(2-chlorophenyl)-6-methyl-5- |
| | (methylsulfonyl)-1,4-dihydropyrimidin-2- |
| | yl)benzo[d]oxazol-2-amine; |
| (244) | (E)-2-(benzo[d]oxazol-2-ylamino)-4-(2- |
| | chlorophenyl)-4,6,7,8-tetrahydroquinazolin- |
| | 5(1H)-one oxime; |
| (245) | 2-(benzo[d]oxazol-2-ylamino)-4-(2- |
| | chlorophenyl)-4,6,7,8-tetrahydropyrido[4,3- |
| | d]pyrimidin-5(1H)-one; |
| (246) | 2-(benzo[d]oxazol-2-ylamino)-4-(2- |
| | chlorophenyl)-N-(1,3,4-thiadiazol-2-yl)-6- |
| | (trifluoromethyl)-1,4-dihydropyrimidine-5- |
| | carboxamide; |
| (247) | 2-(benzo[d]oxazol-2-ylamino)-4-(2- |
| | chlorophenyl)-6-(methoxymethyl)-N-(1,3,4- |
| | thiadiazol-2-yl)-1,4-dihydropyrimidine-5- |
| | carboxamide; |
| (248) | 2-(benzo[d]oxazol-2-ylamino)-4-(2- |
| | chlorophenyl)-6-isobutyl-N-(1,3,4-thiadiazol- |
| | 2-yl)-1,4-dihydropyrimidine-5-carboxamide; |
| (249) | 2-(benzo[d]oxazol-2-ylamino)-4-(2- |
| | chlorophenyl)-6-(pyrazin-2-yl)-N-(1,3,4- |
| | thiadiazol-2-yl)-1,4-dihydropyrimidine-5- |
| | carboxamide; |
| (250) | 2-(benzo[d]oxazol-2-ylamino)-4-(2- |
| | chlorophenyl)-6-isopropyl-N-(1,3,4- |
| | thiadiazol-2-yl)-1,4-dihydropyrimidine-5- |
| | carboxamide; |
| (251) | 2-(benzo[d]oxazol-2-ylamino)-4-(2- |
| | bromophenyl)-6-methyl-N-(1,3,4-thiadiazol- |
| | 2-yl)-1,4-dihydropyrimidine-5-carboxamide; |
| (252) | 7-(benzo[d]oxazol-2-ylamino)-9-methyl- |
| | N-(1,3,4-thiadiazol-2-yl)-6,8- |
| | diazaspiro[4.5]deca-6,9-diene-10- |
| | carboxamide; |
| (253) | 2-(benzo[d]oxazol-2-ylamino)-4-(4- |
| | chloro-1-methyl-1H-pyrazol-3-yl)-6-methyl- |
| | N-(1,3,4-thiadiazol-2-yl)-1,4- |
| | dihydropyrimidine-5-carboxamide; |
| (254) | 2-(benzo[d]oxazol-2-ylamino)-4-(4- |
| | chloro-1H-pyrazol-3-yl)-6-methyl-N-(1,3,4- |
| | thiadiazol-2-yl)-1,4-dihydropyrimidine-5- |
| | carboxamide; |
| (255) | 2-(benzo[d]oxazol-2-ylamino)-4-(3- |
| | bromopyridin-4-yl)-6-methyl-N-(1,3,4- |
| | thiadiazol-2-yl)-1,4-dihydropyrimidine-5- |
| | carboxamide; |
| (256) | 2-(benzo[d]oxazol-2-ylamino)-4-(3- |
| | chloropyridin-2-yl)-6-methyl-N-(1,3,4- |
| | thiadiazol-2-yl)-1,4-dihydropyrimidine-5- |
| | carboxamide; |
| (257) | 2-(benzo[d]oxazol-2-ylamino)-4-(3- |
| | bromopyridin-2-yl)-6-methyl-N-(1,3,4- |
| | thiadiazol-2-yl)-1,4-dihydropyrimidine-5- |
| | carboxamide; |
| (258) | 2-(benzo[d]oxazol-2-ylamino)-4-(4- |
| | bromo-1-methyl-1H-pyrazol-5-yl)-6-methyl- |
| | N-(1,3,4-thiadiazol-2-yl)-1,4- |
| | dihydropyrimidine-5-carboxamide; |
| (259) | 2′-(benzo[d]oxazol-2-ylamino)-6′-methyl- |
| | N-(1,3,4-thiadiazol-2-yl)-1′H- |
| | spiro[bicyclo[4.2.0]octane-7,4′-pyrimidine]- |
| | 1,3,5-triene-5′-carboxamide; |
| (260) | 7-(benzo[d]oxazol-2-ylamino)-9-methyl- |
| | N-(1,3,4-thiadiazol-2-yl)-2-oxa-6,8- |
| | diazaspiro[4.5]deca-6,9-diene-10- |
| | carboxamide; |
| (261) | 2-(benzo[d]oxazol-2-ylamino)-4-(2,4- |
| | dichlorophenyl)-6-methyl-N-(1,3,4-thiadiazol- |
| | 2-yl)-1,4-dihydropyrimidine-5-carboxamide; |
| (262) | 2-(benzo[d]oxazol-2-ylamino)-4-(2- |
| | bromopyridin-3-yl)-6-methyl-N-(1,3,4- |
| | thiadiazol-2-yl)-1,4-dihydropyrimidine-5- |
| | carboxamide; |
| (263) | 2-(benzo[d]oxazol-2-ylamino)-6-methyl- |
| | N-(1,3,4-thiadiazol-2-yl)-4-(2- |
| | (trifluoromethyl)phenyl)-1,4- |
| | dihydropyrimidine-5-carboxamide; |
| (264) | 2-(benzo[d]oxazol-2-ylamino)-4-(2- |
| | chloro-4-methylphenyl)-6-methyl-N-(1,3,4- |
| | thiadiazol-2-yl)-1,4-dihydropyrimidine-5- |
| | carboxamide; |
| (265) | 2-(benzo[d]oxazol-2-ylamino)-4-(4- |
| | bromo-1-methyl-1H-pyrazol-3-yl)-6-methyl- |
| | N-(1,3,4-thiadiazol-2-yl)-1,4- |
| | dihydropyrimidine-5-carboxamide; |
| (266) | 2-(benzo[d]oxazol-2-ylamino)-4-(2- |
| | chloro-4-methoxyphenyl)-6-methyl-N-(1,3,4- |
| | thiadiazol-2-yl)-1,4-dihydropyrimidine-5- |
| | carboxamide; |
| (267) | 2-(benzo[d]oxazol-2-ylamino)-6-methyl- |
| | N-(1,3,4-thiadiazol-2-yl)-4-(o-tolyl)-1,4- |
| | dihydropyrimidine-5-carboxamide; |
| (268) | 2-(benzo[d]oxazol-2-ylamino)-4-(4- |
| | bromo-1H-pyrazol-3-yl)-6-methyl-N-(1,3,4- |
| | thiadiazol-2-yl)-1,4-dihydropyrimidine-5- |
| | carboxamide; |
| (269) | 2-(benzo[d]oxazol-2-ylamino)-4-(2- |
| | chloro-5-methoxyphenyl)-6-methyl-N-(1,3,4- |
| | thiadiazol-2-yl)-1,4-dihydropyrimidine-5- |
| | carboxamide; |
| (270) | 2-(benzo[d]oxazol-2-ylamino)-4-(2,5- |
| | dichlorophenyl)-6-methyl-N-(1,3,4- |
| | thiadiazol-2-yl)-1,4-dihydropyrimidine-5- |
| | carboxamide; |
| (271) | 2-(benzo[d]oxazol-2-ylamino)-4-methyl- |
| | N-(1,3,4-thiadiazol-2-yl)-9-thia-1,3- |
| | diazaspiro[5.5]undeca-1,4-diene-5- |
| | carboxamide; |
| (272) | 2-(benzo[d]oxazol-2-ylamino)-4-methyl- |
| | N-(1,3,4-thiadiazol-2-yl)-9-thia-1,3- |
| | diazaspiro[5.5]undeca-1,4-diene-5- |
| | carboxamide; |
| (273) | 2-(2-(benzo[d]oxazol-2-ylamino)-4-(2- |
| | chlorophenyl)-1,6-dimethyl-1,4- |
| | dihydropyrimidine-5- |
| | carboxamido)isonicotinic acid; |
| (274) | 2-(2-(benzo[d]oxazol-2-ylamino)-4-(2- |
| | chlorophenyl)-1-(ethoxycarbonyl)-6-methyl- |
| | 1,4-dihydropyrimidine-5- |
| | carboxamido)isonicotinic acid; |
| (275) | 2-(2-(benzo[d]oxazol-2-ylamino)-1- |
| | benzoyl-4-(2-chlorophenyl)-6-methyl-1,4- |
| | dihydropyrimidine-5- |
| | carboxamido)isonicotinic acid; |
| (276) | 2-(benzo[d]oxazol-2-ylamino)-4-(2- |
| | chlorophenyl)-5-oxo-1,4,5,6,7,8- |
| | hexahydroquinazoline-7-carboxylic acid; |
| (277) | 2-(benzo[d]oxazol-2-ylamino)-4-(2- |
| | chlorophenyl)-N,N-dimethyl-5-oxo- |
| | 1,4,5,6,7,8-hexahydroquinazoline-7- |
| | carboxamide; |
| (278) | 2-(benzo[d]oxazol-2-ylamino)-4-(2- |
| | chlorophenyl)-N-methyl-5-oxo-1,4,5,6,7,8- |
| | hexahydroquinazoline-7-carboxamide; |
| (279) | 2-(benzo[d]oxazol-2-ylamino)-4-(2- |
| | chlorophenyl)-N-(2-hydroxyethyl)-5-oxo- |
| | 1,4,5,6,7,8-hexahydroquinazoline-7- |
| | carboxamide; |
| (280) | 2-(benzo[d]oxazol-2-ylamino)-4-(2- |
| | chlorophenyl)-N-(2-methoxyethyl)-5-oxo- |
| | 1,4,5,6,7,8-hexahydroquinazoline-7- |
| | carboxamide; |
| (281) | 2-(benzo[d]oxazol-2-ylamino)-4-(2- |
| | chlorophenyl)-5-oxo-N-(1,3,4-thiadiazol-2- |
| | yl)-1,4,5,6,7,8-hexahydroquinazoline-7- |
| | carboxamide; |
| (282) | 2-(benzo[d]oxazol-2-ylamino)-4-(2- |
| | chlorophenyl)-7-(piperazine-1-carbonyl)- |
| | 4,6,7,8-tetrahydroquinazolin-5(1H)-one; |
| (283) | (2-(benzo[d]oxazol-2-ylamino)-4-(2- |
| | chlorophenyl)-5-oxo-1,4,5,6,7,8- |
| | hexahydroquinazoline-7-carbonyl)glycine; |
| (284) | 2-(benzo[d]oxazol-2-ylamino)-4-(2- |
| | chlorophenyl)-N-(2-(dimethylamino)ethyl)-5- |
| | oxo-1,4,5,6,7,8-hexahydroquinazoline-7- |
| | carboxamide; |
| (285) | 2-(4-(2-chlorophenyl)-2-((5- |
| | fluorobenzo[d]oxazol-2-yl)amino)-6-methyl- |
| | 1,4-dihydropyrimidine-5- |
| | carboxamido)isonicotinic acid; |
| (286) | 2-(4-(2-chlorophenyl)-6-methyl-2-((7- |
| | (trifluoro(oxo)-A6-methyl)benzo[d]oxazol-2- |
| | yl)amino)-1,4-dihydropyrimidine-5- |
| | carboxamido)isonicotinic acid; |
| (287) | 2-(4-(3-chloropyridin-4-yl)-2-((6- |
| | fluorobenzo[d]oxazol-2-yl)amino)-6-methyl- |
| | 1,4-dihydropyrimidine-5- |
| | carboxamido)isonicotinic acid; |
| (288) | 2-(4-(3-bromopyridin-4-yl)-2-((6- |
| | fluorobenzo[d]oxazol-2-yl)amino)-6-methyl- |
| | 1,4-dihydropyrimidine-5- |
| | carboxamido)isonicotinic acid; |
| (289) | 2-(4-(2-chloro-4-morpholinophenyl)-2-((6- |
| | fluorobenzo[d]oxazol-2-yl)amino)-6-methyl- |
| | 1,4-dihydropyrimidine-5- |
| | carboxamido)isonicotinic acid; |
| (290) | 2-((7-aminobenzo[d]oxazol-2-yl)amino)- |
| | 4-(2-chloro-4-methylphenyl)-N-((1-ethyl-1H- |
| | pyrazol-4-yl)methyl)-6-methyl-1,4- |
| | dihydropyrimidine-5-carboxamide; |
| (291) | 2-((7-aminobenzo[d]oxazol-2-yl)amino)- |
| | 4-(2-chloro-4-methylphenyl)-6-methyl-N-(1- |
| | (1-methyl-1H-pyrazol-4-yl)ethyl)-1,4- |
| | dihydropyrimidine-5-carboxamide; |
| (292) | 2-((7-aminobenzo[d]oxazol-2-yl)amino)- |
| | 4-(2-chloro-4-methylphenyl)-6-methyl-N- |
| | (pyridin-2-yl)-1,4-dihydropyrimidine-5- |
| | carboxamide; |
| (293) | 2-((7-aminobenzo[d]oxazol-2-yl)amino)- |
| | 4-(2-chloro-4-fluorophenyl)-6-methyl-N-((1- |
| | methyl-1H-pyrazol-4-yl)methyl)-1,4- |
| | dihydropyrimidine-5-carboxamide; |
| (294) | 2-((7-aminobenzo[d]oxazol-2-yl)amino)- |
| | 4-(2,4-dichlorophenyl)-6-methyl-N-((1- |
| | methyl-1H-pyrazol-4-yl)methyl)-1,4- |
| | dihydropyrimidine-5-carboxamide; |
| (295) | 2-(2-((5-amino-6-fluorobenzo[d]oxazol-2- |
| | yl)amino)-4-(2-chlorophenyl)-6-methyl-1,4- |
| | dihydropyrimidine-5- |
| | carboxamido)isonicotinic acid; |
| (296) | 2-(2-((5-amino-6-fluorobenzo[d]oxazol-2- |
| | yl)amino)-4-(2-chloro-4-methoxyphenyl)-6- |
| | methyl-1,4-dihydropyrimidine-5- |
| | carboxamido)isonicotinic acid; |
| (297) | 4-(2-chlorophenyl)-6-methyl-2-((5- |
| | methylbenzo[d]oxazol-2-yl)amino)-N-(5- |
| | (pyridin-3-yl)-1,3,4-thiadiazol-2-yl)-1,4- |
| | dihydropyrimidine-5-carboxamide; |
| (298) | 2-((5-chlorobenzo[d]oxazol-2-yl)amino)- |
| | 4-(2-chlorophenyl)-6-methyl-N-(5-(pyridin-3- |
| | yl)-1,3,4-thiadiazol-2-yl)-1,4- |
| | dihydropyrimidine-5-carboxamide; |
| (299) | 4-(2-chlorophenyl)-6-methyl-2-((6- |
| | methylbenzo[d]oxazol-2-yl)amino)-N-(5- |
| | (pyridin-3-yl)-1,3,4-thiadiazol-2-yl)-1,4- |
| | dihydropyrimidine-5-carboxamide; |
| (300) | 2-(benzo[d]oxazol-2-ylamino)-4-(2- |
| | chlorophenyl)-N-(2-fluorophenyl)-6-methyl- |
| | 1,4-dihydropyrimidine-5-carboxamide; |
| (301) | 2-(benzo[d]oxazol-2-ylamino)-4-(2- |
| | chlorophenyl)-N-(2-hydroxyphenyl)-6- |
| | methyl-1,4-dihydropyrimidine-5- |
| | carboxamide; |
| (302) | 2-(benzo[d]oxazol-2-ylamino)-4-(2- |
| | chlorophenyl)-N-(3-chlorophenyl)-6-methyl- |
| | 1,4-dihydropyrimidine-5-carboxamide; |
| (303) | 2-(benzo[d]oxazol-2-ylamino)-4-(2- |
| | chlorophenyl)-N-(3-fluorophenyl)-6-methyl- |
| | 1,4-dihydropyrimidine-5-carboxamide; |
| (304) | N-(3-(2H-tetrazol-5-yl)phenyl)-2- |
| | (benzo[d]oxazol-2-ylamino)-4-(2- |
| | chlorophenyl)-6-methyl-1,4- |
| | dihydropyrimidine-5-carboxamide; |
| (305) | 2-(benzo[d]oxazol-2-ylamino)-4-(2- |
| | chlorophenyl)-N-(4-fluorophenyl)-6-methyl- |
| | 1,4-dihydropyrimidine-5-carboxamide; |
| (306) | 2-(benzo[d]oxazol-2-ylamino)-4-(2- |
| | chlorophenyl)-N-(4-hydroxyphenyl)-6- |
| | methyl-1,4-dihydropyrimidine-5- |
| | carboxamide; |
| (307) | N-(4-(2H-tetrazol-5-yl)phenyl)-2- |
| | (benzo[d]oxazol-2-ylamino)-4-(2- |
| | chlorophenyl)-6-methyl-1,4- |
| | dihydropyrimidine-5-carboxamide; |
| (308) | 2-(benzo[d]oxazol-2-ylamino)-4-(2- |
| | chlorophenyl)-6-methyl-N-(2- |
| | methylbenzo[d]thiazol-5-yl)-1,4- |
| | dihydropyrimidine-5-carboxamide; |
| (309) | N-(4-(1H-pyrazol-3-yl)phenyl)-2- |
| | (benzo[d]oxazol-2-ylamino)-4-(2- |
| | chlorophenyl)-6-methyl-1,4- |
| | dihydropyrimidine-5-carboxamide; |
| (310) | 2-(benzo[d]oxazol-2-ylamino)-4-(2- |
| | chlorophenyl)-6-methyl-N-(pyridin-3-yl)-1,4- |
| | dihydropyrimidine-5-carboxamide; |
| (311) | methyl 6-(2-(benzo[d]oxazol-2-ylamino)- |
| | 4-(2-chlorophenyl)-6-methyl-1,4- |
| | dihydropyrimidine-5-carboxamido)picolinate; |
| (312) | 2-(benzo[d]oxazol-2-ylamino)-N-(4- |
| | chlorobenzyl)-4-(2-chlorophenyl)-6-methyl- |
| | 1,4-dihydropyrimidine-5-carboxamide; |
| (313) | 2-(2-(benzo[d]oxazol-2-ylamino)-1- |
| | (carboxymethyl)-4-(2-chlorophenyl)-6- |
| | methyl-1,4-dihydropyrimidine-5- |
| | carboxamido)isonicotinic acid; |
| (314) | (2-(benzo[d]oxazol-2-ylamino)-4-(2- |
| | chlorophenyl)-6-methyl-1,4- |
| | dihydropyrimidin-5-yl)(3- |
| | (hydroxymethyl)piperidin-1-yl)methanone; |
| (315) | 1-(2-(benzo[d]oxazol-2-ylamino)-4-(2- |
| | chlorophenyl)-6-methyl-1,4- |
| | dihydropyrimidine-5-carbonyl)piperidine-3- |
| | carboxylic acid; |
| (316) | 2-(benzo[d]oxazol-2-ylamino)-4-(2- |
| | chlorophenyl)-6-isobutyl-1,4,6,7-tetrahydro- |
| | 5H-pyrrolo[3,4-d]pyrimidin-5-one; |
| (317) | 2-(benzo[d]oxazol-2-ylamino)-4-(2- |
| | chlorophenyl)-6-phenyl-1,4,6,7-tetrahydro- |
| | 5H-pyrrolo[3,4-d]pyrimidin-5-one; |
| (318) | 2-(2-(benzo[d]oxazol-2-ylamino)-4-(3- |
| | bromopyridin-4-yl)-6-methyl-1,4- |
| | dihydropyrimidine-5- |
| | carboxamido)isonicotinic acid; |
| (319) | 2-(benzo[d]oxazol-2-ylamino)-4-(3- |
| | bromopyridin-4-yl)-6-methyl-N-((1-methyl- |
| | 1H-imidazol-4-yl)methyl)-1,4- |
| | dihydropyrimidine-5-carboxamide; |
| (320) | 2′-(benzo[d]oxazol-2-ylamino)-6′-methyl- |
| | N-(1,3,4-thiadiazol-2-yl)-1′H- |
| | spiro[bicyclo[4.2.0]octane-7,4′-pyrimidine]- |
| | 1(6),2,4-triene-5′-carboxamide; |
| (321) | 2-(benzo[d]oxazol-2-ylamino)-6-benzyl-4- |
| | (2-chlorophenyl)-1,4,6,7-tetrahydro-5H- |
| | pyrrolo[3,4-d]pyrimidin-5-one; |
| (322) | 1-(2-(benzo[d]oxazol-2-ylamino)-4-(2- |
| | chlorophenyl)-6-methyl-1,4- |
| | dihydropyrimidine-5-carbonyl)piperidine-4- |
| | carboxylic acid; |
| (323) | ethyl 1-(2-(benzo[d]oxazol-2-ylamino)-4- |
| | (2-chlorophenyl)-6-methyl-1,4- |
| | dihydropyrimidine-5-carbonyl)piperidine-3- |
| | carboxylate; |
| (324) | ethyl 1-(2-(benzo[d]oxazol-2-ylamino)-4- |
| | (2-chlorophenyl)-6-methyl-1,4- |
| | dihydropyrimidine-5-carbonyl)piperidine-4- |
| | carboxylate; or |
| (325) | (2-(benzo[d]oxazol-2-ylamino)-4-(2- |
| | chlorophenyl)-6-methyl-1,4- |
| | dihydropyrimidin-5-yl)(4- |
| | (hydroxymethyl)piperidin-1-yl)methanone. |

28. A method for inhibiting a galactokinase in a subject in need thereof, the method comprising administering to the subject an effective amount of a compound of any one of claims 1-27, or a pharmaceutically acceptable salt, hydrate, solvate, prodrug, stereoisomer, or tautomer thereof.

29. A method for inhibiting a galactokinase in a subject in need thereof, the method comprising administering to the subject a therapeutically effective amount of a pharmaceutical composition comprising a compound of any one of claims 1-27, or a pharmaceutically acceptable salt, hydrate, solvate, prodrug, stereoisomer, or tautomer thereof, and optionally, a pharmaceutically acceptable carrier.

30. A method for treating or prophylaxis of a disease associated with the galactokinase (GALK1) enzyme or the PTEN/PI3K/AKT pathway in a subject in need thereof, the method comprising administering to the subject a therapeutically effective amount of a compound of any one of claims 1-27, or a pharmaceutically acceptable salt, hydrate, solvate, prodrug, stereoisomer, or tautomer thereof, and optionally, a pharmaceutically acceptable carrier.

31. A method for treating or prophylaxis of classic galactosemia in a subject in need thereof, the method comprising administering to the subject a therapeutically effective amount of a compound of any one of claims 1-27, or a pharmaceutically acceptable salt, hydrate, solvate, prodrug, stereoisomer, or tautomer thereof.

32. A method for treating or prophylaxis of liver failure, coagulopathy, coma, or death mediated by a the galactokinase (GALK1) enzyme or the PTEN/PI3K/AKT pathway in a subject in need thereof, the method comprising administering to the subject a therapeutically effective amount of a compound of any one of claims 1-27, or a pharmaceutically acceptable salt, hydrate, solvate, prodrug, stereoisomer, or tautomer thereof, and optionally, a pharmaceutically acceptable carrier.

33. Use of a compound of any one of claims 1-27 for treating or prophylaxis of a disease associated with the galactokinase (GALK1) enzyme or the PTEN/PI3K/AKT pathway in a subject in need thereof, the method comprising administering to the subject a therapeutically effective amount of the compound or a pharmaceutically acceptable salt, hydrate, solvate, prodrug, stereoisomer, or tautomer thereof, and optionally, a pharmaceutically acceptable carrier.

34. A kit comprising a dosage form of a compound of any one of claims 1-27; at least one moisture proof dispensing receptacle comprising blister or strip packs, an aluminum blister, a transparent or opaque polymer blister with pouch, polypropylene tubes, colored blister materials, tubes, bottles, and bottles optionally containing a child-resistant feature, optionally comprising a desiccant, such as a molecular sieve or silica gel; and optionally an insert comprising instructions or prescribing information for the compound or directions for administration or any contraindications.

35. A method for manufacturing a compound of any one of claims 1-27, the method comprising performing any one of the synthesis reactions described herein.

36. A compound produced by the method of claim 35.

37. A method for using Structure-Activity Relationship (SAR) analyses to develop compounds with enhanced activity comprising any one of claims 1-27.
